(19) United States
(12) Patent Application Publication
(10) Pub. No.: US 2026/0165024 A1
(43) Pub. Date: Jun. 11, 2026

(54) ORGANIC COMPOUND, MIXTURE, COMPOSITION, ORGANIC ELECTRONIC DEVICE, AND DISPLAY PANEL

(71) Applicant: GUANGZHOU CHINARAY OPTOELECTRONIC MATERIALS LTD., Guangzhou (CN)

(72) Inventors: Yuanzuo ZOU, Guangzhou (CN); Shihao ZHOU, Guangzhou (CN); Mingrui PU, Guangzhou (CN); Ruifeng HE, Guangzhou (CN); Jingyao SONG, Guangzhou (CN)

(73) Assignee: GUANGZHOU CHINARAY OPTOELECTRONIC MATERIALS LTD., Guangzhou (CN)

(21) Appl. No.: 19/028,235

(22) Filed: Jan. 17, 2025

(30) Foreign Application Priority Data
Dec. 6, 2024 (CN) 202411799985.9

(51) Int. Cl. H10K 85/60; C07D 405/14; C07D 409/14; C07D 471/04; C09K 11/02; H10K 50/12; H10K 101/30

(52) U.S. Cl. CPC H10K 85/654; C07D 405/14; C07D 409/14; C07D 471/04; C09K 11/02; H10K 85/615; H10K 85/6572; H10K 85/6574; H10K 85/6576; H10K 50/12; H10K 2101/30

(57) ABSTRACT

An organic compound has a chemical structure represented by formula (1), in which X¹ is selected from O or S; X² is selected from O, S, or NR³; Z¹, Z², and Z³ are independently selected from CH or N, and at least one of Z¹, Z², and Z³ is N; Ar¹ and Ar² are independently selected from a substituted or unsubstituted aromatic group having 6 to 20 carbon atoms, or a substituted or unsubstituted heteroaromatic group having 5 to 20 carbon atoms; R¹ is selected from a hydrogen atom or a deuterium atom; and R² is selected from a hydrogen atom, a deuterium atom, a substituted or unsubstituted aromatic group having 6 to 20 carbon atoms, or a substituted or unsubstituted heteroaromatic group having 5 to 20 carbon atoms.

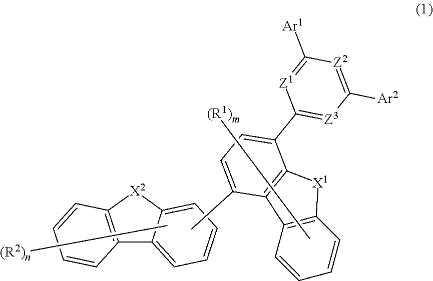

### CROSS-REFERENCE TO RELATED APPLICATION

This application claims the priority to and benefit of Chinese Patent Application No. 202411799985.9, filed on Dec. 6, 2024, the disclosure of which is incorporated herein by reference in its entirety.

### TECHNICAL FIELD

The disclosure relates to the field of display, and in particular, to an organic compound, a mixture, a composition, an organic electronic device, and a display panel.

### BACKGROUND

At present, an organic electroluminescent device usually includes an anode, a cathode, and an organic layer disposed between the anode and the cathode, and organic substances in the organic layer can convert electrical energy into light energy to achieve organic electroluminescence. In order to improve luminous efficiency and service life of the organic electroluminescent device, the organic layer is often composed of multiple layers, and organic compounds in different layers are different. The organic layer includes, but not limited to, a hole injection layer, a hole transport layer, a light-emitting auxiliary layer, a light-emitting layer, a hole blocking layer, an electron transport layer, and an electron injection layer. During the operation of the organic electroluminescent device, in response to a voltage being applied between the anode and the cathode, holes in the anode are injected into the organic layer, electrons in the cathode are injected into the organic layer, the injected holes meet with the injected electrons to form excitons, and the excitons emit light when transitioning back to the ground state through radiation, thereby achieving luminescence of the organic electroluminescent device. The organic electroluminescent device has wide application prospect due to its characteristics of autonomous luminescence, high brightness, high efficiency, low driving voltage, wide viewing angle, high contrast ratio, high responsiveness, and the like.

Materials of the light-emitting layer in the organic electroluminescent device often need to have high quantum efficiency, carrier mobility, and stability. In order to avoid aggregation and quenching of excitons, a host material doped with a guest material is used as a material of the light-emitting layer to achieve effective utilization of energy of the excitons. However, existing materials of the light-emitting layer have insufficient transport property and lower stability, so that the driving voltage, the luminous efficiency, and the serve life of the organic electroluminescent device cannot meet the requirements, which may limit the use of the organic electroluminescent device.

### SUMMARY

Some embodiments of the disclosure provide an organic compound having a chemical structure represented by the following formula (1):

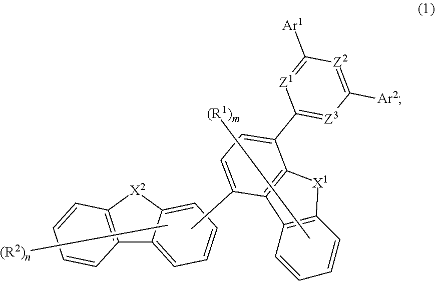

in which X¹ is selected from O or S;
X² is selected from O, S, or NR³;
Z¹, Z², and Z³ are independently selected from CH or N, and at least one of Z¹, Z², and Z³ is N;
Ar¹ and Ar² are independently selected from a substituted or unsubstituted aromatic group having 6 to 20 carbon atoms, or a substituted or unsubstituted heteroaromatic group having 5 to 20 carbon atoms;
R¹ is selected from a hydrogen atom or a deuterium atom;
R² is selected from a hydrogen atom, a deuterium atom, a substituted or unsubstituted aromatic group having 6 to 20 carbon atoms, or a substituted or unsubstituted heteroaromatic group having 5 to 20 carbon atoms;
R³ is selected from a substituted or unsubstituted aromatic group having 6 to 18 carbon atoms, or a substituted or unsubstituted heteroaromatic group having 5 to 18 carbon atoms;
m is any integer from 1 to 6; and
n is any integer from 1 to 7.

Some embodiments of the disclosure provide a mixture including the organic compound as described above and an organic functional material, and the organic functional material is selected from at least one of a hole injection material, a hole transport material, an electron injection material, an electron transport material, a light-emitting auxiliary material, a hole blocking material, a guest material, a host material, and quantum dots.

Some embodiments of the disclosure provide a composition including an organic solvent and the organic compound as described above.

Some embodiments of the disclosure provide an organic electronic device, including:
a first electrode;
a second electrode disposed opposite to the first electrode; and
an organic functional layer disposed between the first electrode and the second electrode, in which a material of the organic functional layer includes the organic compound as described above.

Some embodiments of the disclosure provide a display panel including the organic electronic device as described above.

### BRIEF DESCRIPTION OF THE DRAWINGS

In order to explain technical solutions in embodiments of the disclosure more clearly, the following will briefly introduce the drawings needed to be used in description of the embodiments. Apparently, the drawings in the following description are only some embodiments of the disclosure. For ordinary skilled in the art, other drawings can be obtained from these drawings without paying creative effort.

In order to understand the disclosure and beneficial effects thereof more completely, the following will be described in combination with the drawings. In the following description, the same reference numerals indicate the same elements.

FIG. 1 is a schematic structural diagram of an organic electronic device provided by some embodiments of the disclosure.

### REFERENCE CHARACTERS

100, organic electronic device; 1, substrate; 11, first electrode; 12, hole injection layer; 13, hole transport layer; 14, light-emitting auxiliary layer; 15, light-emitting layer; 16, electron transport layer; 17, electron injection layer; and 18, second electrode.

### DETAILED DESCRIPTION

The following will provide a clear and complete description of the technical solutions in the embodiments of the disclosure, in conjunction with the drawings. Apparently, the described embodiments are only a part of the embodiments of the disclosure, not all of them. Based on the embodiments of the disclosure, all other embodiments obtained by those skilled in the art without creative labor are within the scope of protection of the disclosure. In addition, it should be understood that specific embodiments described herein are only used to explain and illustrate the disclosure and are not intended to limit the disclosure.

In the disclosure, unless otherwise specified, the directional terms, such as “on” and “below”, generally refer to upward and downward directions of the device, respectively, in actual use or working state, or in particular directions in the drawings; and terms “inside” and “outside” are relative to the contour of the devices shown in the drawings.

In the disclosure, the term “optionally” refers to optional options, that is, to any one selected from two parallel solutions of “having” and “absent”. If there are multiple “optionally” in a technical solution, unless otherwise specified, and there is no contradiction or mutual restriction, each “optionally” is independent.

In the disclosure, a technical feature described in an open format includes both a closed technical solution consisting of the listed features and an open technical solution including the listed features.

In the disclosure, an aryl group, an aromatic group, and an aromatic ring system have the same meaning and may be interchanged.

In the disclosure, a heteroaryl group, a heteroaromatic group, and a heteroaromatic ring system have the same meaning and may be interchanged.

In the disclosure, a cycloalkyl group and a cyclic alkyl group have the same meaning and may be interchanged.

In the disclosure, “substituted” means that a hydrogen atom in a group to be substituted is substituted by a substituent group.

In the disclosure, a same substituent group at different substituent sites may be independently selected from the same group or different groups. For example, if a formula includes a plurality of R groups, each of the R groups may be independently selected from the same group or different groups.

In the disclosure, “number of ring atoms” refers to a number of atoms constituting a ring of a structural compound obtained by atomic bonding, for example, a monocyclic compound, a fused ring compound, a cross-linked compound, a carbon ring compound, or a heterocyclic compound. In a ring substituted by a substituent group, the atoms contained in the substituent group are not included in the atoms forming the ring. The same applies to “number of ring atoms” described below unless otherwise specified. For example, the number of ring atoms in benzene is 6, the number of ring atoms in naphthalene is 10, and the number of ring atoms in thiophene is 5.

In the disclosure, “substituted or unsubstituted” means that a defined group may be substituted or not be substituted. When the defined group is substituted, it can be understood that the defined group may be substituted by one or more substituent R groups. The R groups are independently selected from, but not limited to, a deuterium atom, a cyanoyl group, an isocyanoyl group, a nitro group, a halogen atom, an alkyl group having 1 to 20 carbon atoms, a heterocyclic group having 3 to 20 ring atoms, an aromatic group having 6 to 20 ring atoms, a heteroaromatic group having 5 to 20 ring atoms, —NR′R″, a silanyl group, a carbonyl group, an alkoxycarbonyl group, an aryloxycarbonyl group, an aminoformyl group, a haloformyl group, a formyl group, an isocyanate group, a thiocyanate group, an isothiocyanate group, a hydroxyl group, or a trifluoromethyl group, and the groups may further be substituted by acceptable substituent groups in the art. Understandably, R′ and R″ in the NR′R″ are independently selected from, but not limited to, a hydrogen atom, a deuterium atom, a cyanoyl group, an isocyanoyl group, a nitro group, a halogen atom, an alkyl group having 1 to 10 carbon atoms, a heterocyclic group having 3 to 20 ring atoms, an aromatic group having 6 to 20 ring atoms, or a heteroaromatic group having 5 to 20 ring atoms. In some embodiments, R is selected from, but not limited to, a deuterium atom, a cyanoyl group, an isocyanoyl group, a nitro group, a halogen atom, an alkyl group having 1 to 10 carbon atoms, a heterocyclic group having 3 to 10 ring atoms, an aromatic group having 6 to 20 ring atoms, a heteroaromatic group having 5 to 20 ring atoms, a silanyl group, a carbonyl group, an alkoxycarbonyl group, an aryloxycarbonyl group, an aminoformyl group, a haloformyl group, a formyl group, an isocyanate group, a thiocyanate group, an isothiocyanate group, a hydroxyl group, or a trifluoromethyl group, and the groups may further be substituted by acceptable substituent groups in the art.

In the disclosure, “an aryl group or an aromatic group” refers to an aromatic hydrocarbon group derived from a basis of an aromatic ring compound removing one hydrogen atom. The aromatic hydrocarbon group may be a monocyclic aryl group, a fused ring aryl group, or a polycyclic aryl group. For a polycyclic ring type, at least one ring is an aromatic ring system. For example, “a substituted or unsubstituted aryl group having 6 to 40 ring atoms” refers to an aryl group having 6 to 40 ring atoms, a substituted or unsubstituted aryl group having 6 to 30 ring atoms, a substituted or unsubstituted aryl group having 6 to 18 ring atoms, or a substituted or unsubstituted aryl group having 6 to 14 ring atoms, and the aryl group is optionally further substituted. Examples of the aryl group or the aromatic group include, but not limited to, a phenyl group, a biphenyl group, a triphenyl group, a naphthyl group, an anthracyl group, a phenanthryl group, a fluoranthenyl group, a triphenylenyl group, a pyrenyl group, a perylenyl group, a tetraphenyl group, a fluorenyl group, a perylenyl group, an acenaphthenyl group, and derivatives thereof. Understandably, one or more aryl groups may further be disconnected by a short non-aromatic unit (for example, a non-hydrogenium atom contenting less than 10%, such as C, N, or O). In some embodiments, an acenaphthenyl group, a fluorenyl group, a 9,9′-diarylfluorenyl group, a triarylaminyl group, and a diaryl ether system may be further included in the definition of the aryl group.

In the disclosure, “a heteroaryl group or a heteroaromatic group” refers to a basis of an aryl group with at least one carbon atom replaced by a non-carbon atom, and the non-carbon atom may be N, O, S, or the like. For example, “a substituted or unsubstituted heteroaryl group having 5 to 40 ring atoms” refers to a heteroaryl group having 5 to 40 ring atoms, a substituted or unsubstituted heteroaryl group having 6 to 30 ring atoms, a substituted or unsubstituted heteroaryl group having 6 to 18 ring atoms, or a substituted or unsubstituted heteroaryl group having 6 to 14 ring atoms, and the heteroaryl group is optionally further substituted. Examples of the heteroaryl group or the heteroaromatic group include, but not limited to, a thienyl group, a furyl group, a pyrrolyl group, an imidazolyl group, a diazolyl group, a triazolyl group, a pyridyl group, a bipyridyl group, a pyrimidinyl group, a triazinyl group, an acridinyl group, a pyridazinyl group, a pyrazinyl group, a quinolinyl group, an isoquinolinyl group, a quinazolinyl group, a quinoxalinyl group, a phthalazinyl group, a pyridinopyrimidinyl group, a pyridinopyrazinyl group, a benzothiophenyl group, a benzofuranyl group, an indolyl group, a pyrroloimidazolyl group, a pyrrolopyrrolyl group, a thiophenopyrrolyl group, a thiophenothiophenyl group, a furanopyrrolyl group, a furanofuranyl group, a thiophenofuranyl group, a benzoisoxazolyl group, a benzoisothiazolyl group, a benzimidazolyl group, an ortho-diazonaphthalyl group, a phenanthridinyl group, a perimidinyl group, a quinazolinketone group, a dibenzothiophenyl group, a dibenzofuranyl group, a carbazolyl group, and derivatives thereof.

In the disclosure, “an alkyl group” refers to a linear alkyl group, a branched alkyl group, or a cyclic alkyl group. The number of carbon atoms in the alkyl group may range from 1 to 50, 1 to 30, 1 to 20, 1 to 10, or 1 to 6. The term having the alkyl group, such as “a C₁₋₉ alkyl group”, refers to an alkyl group having 1-9 carbon atoms. The C₁₋₉ alkyl group is independently selected from a C₁ alkyl group, a C₂ alkyl group, a C₃ alkyl group, a C₄ alkyl group, a C₅ alkyl group, a C₆ alkyl group, a C₇ alkyl group, a C₈ alkyl group, or a C₉ alkyl group at each occurrence. Examples of the alkyl group include, but not limited to, methyl, ethyl, n-propyl, isopropyl, n-butyl, sec-butyl, tert-butyl, isobutyl, 2-ethylbutyl, 3,3-dimethylbutyl, n-pentyl, isopentyl, neopentyl, tert-pentyl, cyclopentyl, 1-methylpentyl, 3-methylpentyl, 2-ethylpentyl, 4-methyl-2-pentyl, n-hexyl, 1-methylhexyl, 2-ethylhexyl, 2-butyhexyl, cyclohexyl, 4-methylcyclohexyl, 4-tert-butylcyclohexyl, n-heptyl, 1-methylheptyl, 2,2-dimethylheptyl, 2-ethylheptyl, 2-butyl-heptyl, n-octyl, tert-octyl, 2-ethyloctyl, 2-butyl-octyl, 2-hexyl-octyl, 3,7-dimethyloctyl, cyclooctyl, n-nonyl, n-decanyl, an adamantine group, 2-ethyldecyl, 2-butyldecyl 2-hexyldecyl, 2-octyldecyl, n-undecyl, n-dodecyl, 2-ethyldodecyl 2-butyldodecyl, 2-hexyldodecyl, 2-octyldodecyl, n-tridecyl, n-tetradecyl, n-pentadecyl, n-hexadecyl, 2-ethylhexadecyl, 2-butylhexadecyl, 2-hexylhexadecyl, 2-octylhexadecyl, n-heptadecyl, n-octadecyl, n-nonadecyl, n-eicosyl, 2-ethyleicosyl, 2-butyeicosyl, 2-hexyleicosyl, 2-octyleicosyl, n-heneicosyl, n-docosyl, n-tricosyl, n-tetracosyl, n-pentacosyl, n-hexacosyl, n-heptacosyl, n-octacosyl, n-nonacosyl, n-triacontyl, and the like.

In the disclosure, “an amino group” refers to a derivative of amine and has a structural feature of a group represented by formula —N(X)₂, in which X is independently selected from a hydrogen atom, a substituted or unsubstituted alkyl group, a substituted or unsubstituted cycloalkyl group, a substituted or unsubstituted heterocyclic group, or the like. Examples of the amino group include, but not limited to, —NH₂, —N(alkyl)₂, —NH(alkyl), —N(cycloalkyl)₂, —NH(cycloalkyl), —N(heterocyclic)₂, —NH(heterocyclic), —N(aryl)₂, —NH(aryl), —N(alkyl)(aryl), —N(alkyl)(heterocyclic), —N(cycloalkyl)(heterocyclic), —N(aryl)(heteroaryl), —N(alkyl)(heteroaryl), and the like.

In the disclosure, “a cycloalkyl group” or “a cyclic alkyl group” refers to a monovalent group having one or more saturated rings in which all ring atoms are carbon atoms. “Alkyl groups” in the “cycloalkyl group” and the “cyclic alkyl group” have the same meaning as the “alkyl group” as defined above.

In the disclosure, “a heterocyclic group”, “heterocyclic”, or “a heterocyclic ring” refers to a non-aromatic cyclic group that is fully saturated or partially unsaturated. The non-aromatic cyclic group has one or more heteroatoms, such as an oxygen atom (O), a sulfur atom (S), a silicon atom (Si), or a nitrogen atom (N), in which N and S are optionally oxidized, and N is optionally quaternized. In some embodiments, the heterocyclic group is connected to any atom or any carbon atom in a ring or a ring system, and the heterocyclic group is unsubstituted or substituted by one or more aryl groups as described above.

In the disclosure, unless otherwise specified, a hydroxyl group refers to —OH, a carboxyl group refers to —COOH, a carbonyl group refers to —C(═O)—, an amino group refers to —NH₂, a formyl group refers to —C(═O)H, a haloformyl group refers to —C(═O)Z (Z refers to a halogen atom, such as F, Cl, Br, or I), a carbamoyl group refers to —C(═O)NH₂, an isocyanate group refers to —NCO, and an isothiocyanate group refers to —NCS.

In the disclosure, “an alkoxy group” refers to a group having a structure of “—O-alkyl”, that is, the alkyl group as defined above is connected to other groups through an oxygen atom to form the alkoxy group. Examples of the alkoxy group include, but not limited to, a methoxy group (—O—CH₃ or —OMe), an ethoxy group (—O—CH₂CH₃ or —OEt), and a tert-butoxy group (—O—C(CH₃)₃ or —OtBu).

In the disclosure, “*” connected to a single bond indicates a linking site or a fused site.

In the disclosure, when a linking site in a group is not specified, it means that any of connectable sites in the group may be selected as the linking site.

In the disclosure, when a fused site in a group is not specified, it means that any of fusible sites in the group may be selected as the fused site. For example, two or more adjacent sites in the group form a fused site.

In the disclosure, when there are more than one substituent groups with the same symbol on the same group, the substituent groups may be the same or different. For example, in formula

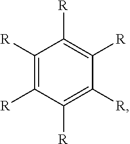

six R groups in a benzene ring may be the same or different.

In the disclosure, a single bond connected to a substituent group and penetrated a corresponding ring indicates that the substituent group may be connected to any site of the ring. For example,

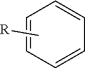

means that R may be connected to any substituent site of the benzene ring, and

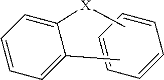

means that

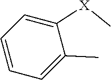

may be connected to any substituent site of

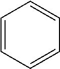

to form two rings connected to each other.

Some embodiments of the disclosure provide an organic compound having a chemical structure represented by the following formula (1):

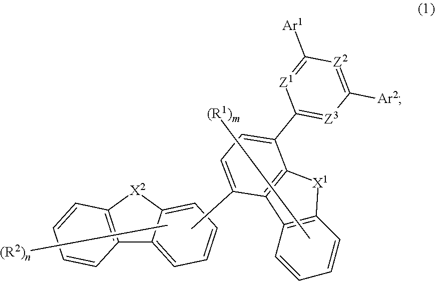

in which X¹ is selected from O or S;
X² is selected from O, S, or NR³;
Z¹, Z², and Z³ are independently selected from CH or N, and at least one of Z¹, Z², and Z³ is N;
Ar¹ and Ar² are independently selected from a substituted or unsubstituted aromatic group having 6 to 20 carbon atoms, or a substituted or unsubstituted heteroaromatic group having 5 to 20 carbon atoms;
R¹ is selected from a hydrogen atom or a deuterium atom;
R² is selected from a hydrogen atom, a deuterium atom, a substituted or unsubstituted aromatic group having 6 to 20 carbon atoms, or a substituted or unsubstituted heteroaromatic group having 5 to 20 carbon atoms;
R³ is selected from a substituted or unsubstituted aromatic group having 6 to 18 carbon atoms, or a substituted or unsubstituted heteroaromatic group having 5 to 18 carbon atoms;
m is any integer from 1 to 6; and
n is any integer from 1 to 7.

In the embodiments of the disclosure, the structure of the organic compound is composed of multiple conjugated planar rigid functional groups rich in electrons, such as a heterocyclic ring containing N, and dibenzoheterocyclic rings with at least one heteroatom of O and S, so that the organic compound has stronger electron transport property and structural stability. When the organic compound is used in a light-emitting layer in combination with a hole transport type host material, effective transport of excited state energy can be achieved, which is beneficial to improving luminous efficiency and service life of an organic electronic device using the organic compound. In addition, when Z¹, Z², and Z³ are all N, the heterocyclic ring containing N is directly connected to the no. 4 site of a first dibenzoheterocyclic ring, and the no. 1 site of the first dibenzoheterocyclic ring is connected to a second dibenzoheterocyclic ring. The double substitution of the first dibenzoheterocyclic ring makes the molecular conformation of the organic compound more compact, and a dihedral angle between a plane of the heterocyclic ring containing N and a plane of the first and second dibenzoheterocyclic rings is reduced, so that the distribution region of the lowest unoccupied molecular orbital (LUMO) energy level of the organic compound tends to be more planar, thereby further improving the electron transport property and structural stability of the organic compound. Therefore, the luminous efficiency and the service life of the organic electronic device using the organic compound of the embodiments can be improved.

In some embodiments, Z¹, Z², and Z³ are all N.

In some embodiments, Ar¹ and Ar² are independently selected from a phenyl group, a deuterated phenyl group, a biphenyl group, a triphenyl group, a naphthyl group, a phenanthryl group, a pyridyl group, a pyrimidinyl group, a pyrazinyl group, a quinolinyl group, an isoquinolinyl group, a phenanthrolinyl group, or any combination thereof.

In some embodiments, Ar¹ and Ar² are independently selected from a phenyl group, a biphenyl group, a pyridyl group, an isoquinolinyl group, or any combination thereof.

In some embodiments, R² is selected from a hydrogen atom, a deuterium atom, a phenyl group, a deuterated phenyl group, a biphenyl group, a triphenyl group, a naphthyl group, a phenanthryl group, a pyridyl group, a pyrimidinyl group, a pyrazinyl group, a quinolinyl group, an isoquinolinyl group, a phenanthrolinyl group, or any combination thereof at each occurrence.

In some embodiments, R² is independently selected from a hydrogen atom, a deuterium atom, a phenyl group, a biphenyl group, a naphthyl group, a phenanthryl group, a deuterated phenyl group, or any combination thereof at each occurrence.

In some embodiments, R³ is selected from a phenyl group, a biphenyl group, a deuterated phenyl group, a pyridyl group, or any combination thereof.

In some embodiments, R³ is selected from a phenyl group, a biphenyl group, a pyridyl group, or any combination thereof.

In some embodiments, the organic compound has the chemical structure represented by any one of the following formulae (P-1) to (P-160), formulae (Q-1) to (Q-160), formulae (R-1) to (R-128), and formulae (S-1) to (S-100):

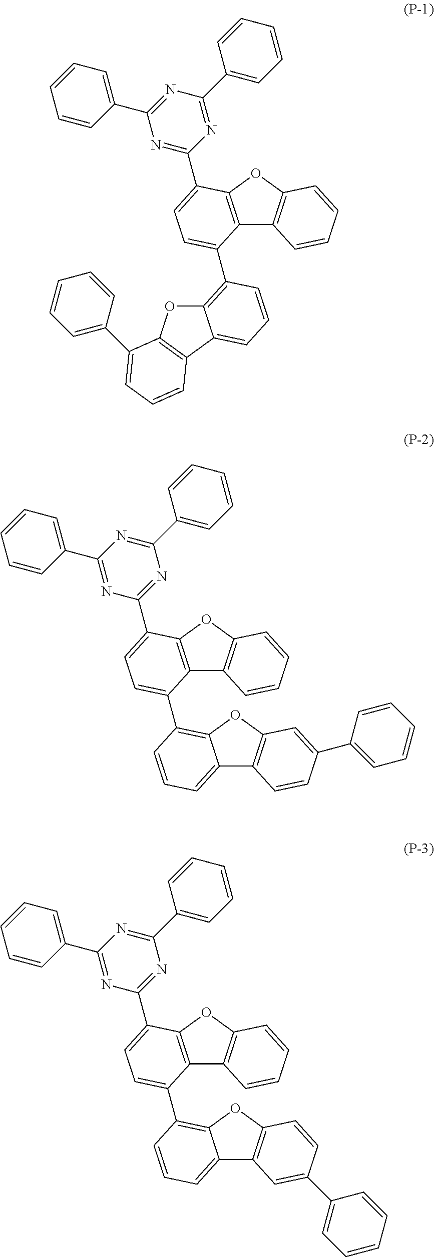 [SMILES: c1ccc(-c2nc(-c3ccccc3)nc(-c3ccc(-c4cccc5c4oc4c(-c6ccccc6)cccc45)c4c3oc3ccccc34)n2)cc1 ; c1ccc(-c2ccc3c(c2)oc2c(-c4ccc(-c5nc(-c6ccccc6)nc(-c6ccccc6)n5)c5oc6ccccc6c45)cccc23)cc1 ; c1ccc(-c2ccc3oc4c(-c5ccc(-c6nc(-c7ccccc7)nc(-c7ccccc7)n6)c6oc7ccccc7c56)cccc4c3c2)cc1]

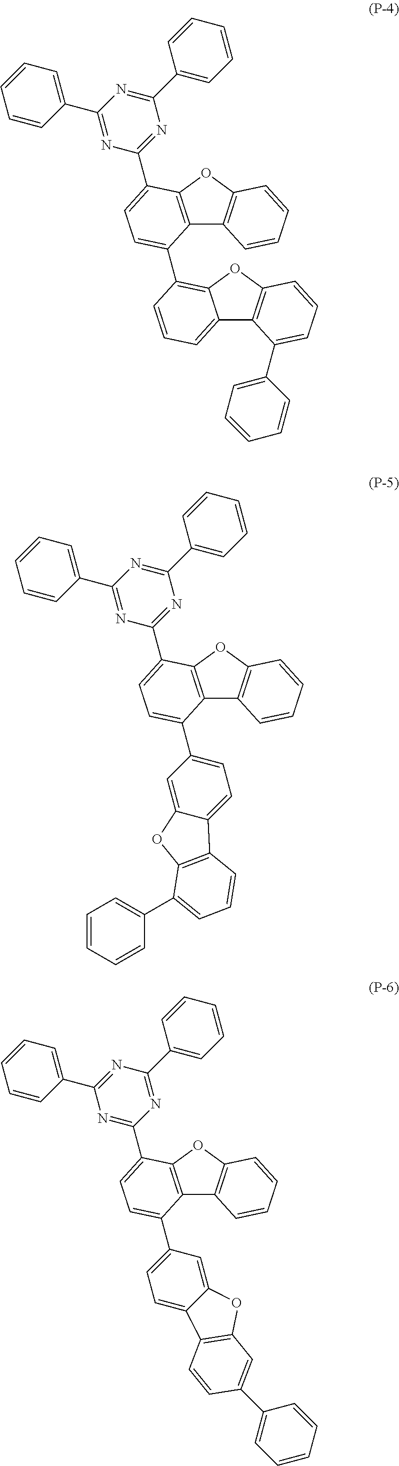 [SMILES: c1ccc(-c2nc(-c3ccccc3)nc(-c3ccc(-c4cccc5c4oc4cccc(-c6ccccc6)c45)c4c3oc3ccccc34)n2)cc1 ; c1ccc(-c2nc(-c3ccccc3)nc(-c3ccc(-c4ccc5c(c4)oc4c(-c6ccccc6)cccc45)c4c3oc3ccccc34)n2)cc1 ; c1ccc(-c2ccc3c(c2)oc2cc(-c4ccc(-c5nc(-c6ccccc6)nc(-c6ccccc6)n5)c5oc6ccccc6c45)ccc23)cc1]

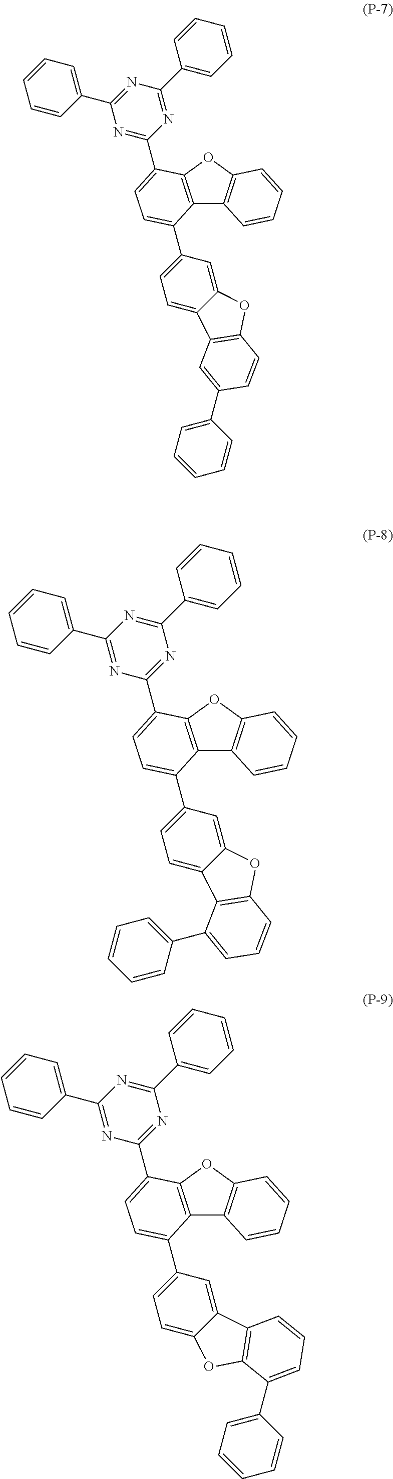

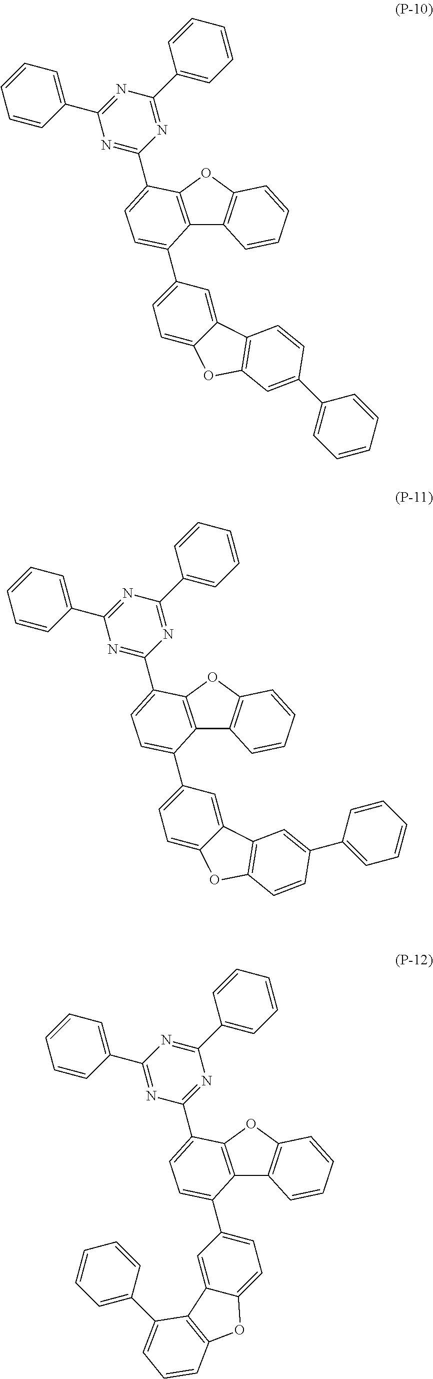 [SMILES: c1ccc(-c2ccc3c(c2)oc2ccc(-c4ccc(-c5nc(-c6ccccc6)nc(-c6ccccc6)n5)c5oc6ccccc6c45)cc23)cc1 ; c1ccc(-c2ccc3oc4ccc(-c5ccc(-c6nc(-c7ccccc7)nc(-c7ccccc7)n6)c6oc7ccccc7c56)cc4c3c2)cc1 ; c1ccc(-c2nc(-c3ccccc3)nc(-c3ccc(-c4ccc5oc6cccc(-c7ccccc7)c6c5c4)c4c3oc3ccccc34)n2)cc1]

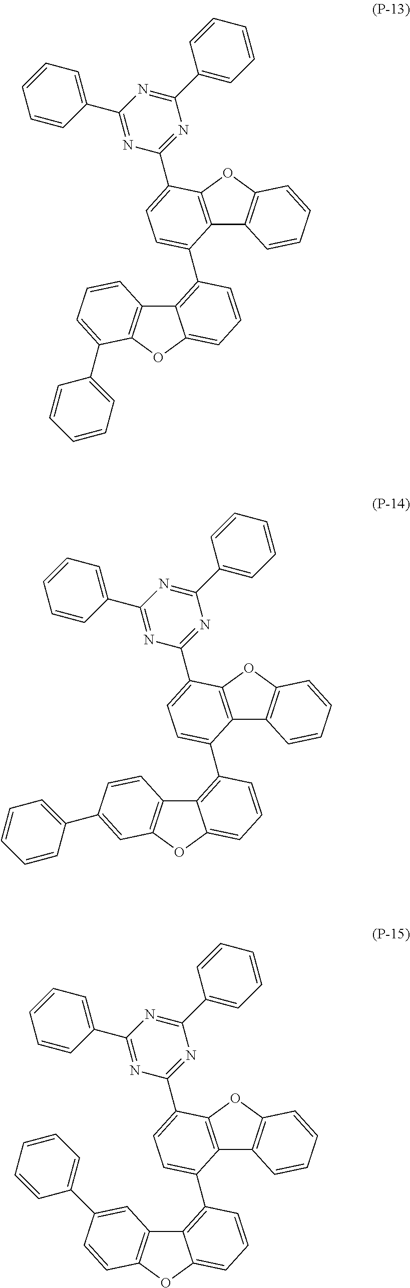 [SMILES: c1ccc(-c2nc(-c3ccccc3)nc(-c3ccc(-c4cccc5oc6c(-c7ccccc7)cccc6c45)c4c3oc3ccccc34)n2)cc1 ; c1ccc(-c2ccc3c(c2)oc2cccc(-c4ccc(-c5nc(-c6ccccc6)nc(-c6ccccc6)n5)c5oc6ccccc6c45)c23)cc1 ; c1ccc(-c2ccc3oc4cccc(-c5ccc(-c6nc(-c7ccccc7)nc(-c7ccccc7)n6)c6oc7ccccc7c56)c4c3c2)cc1]

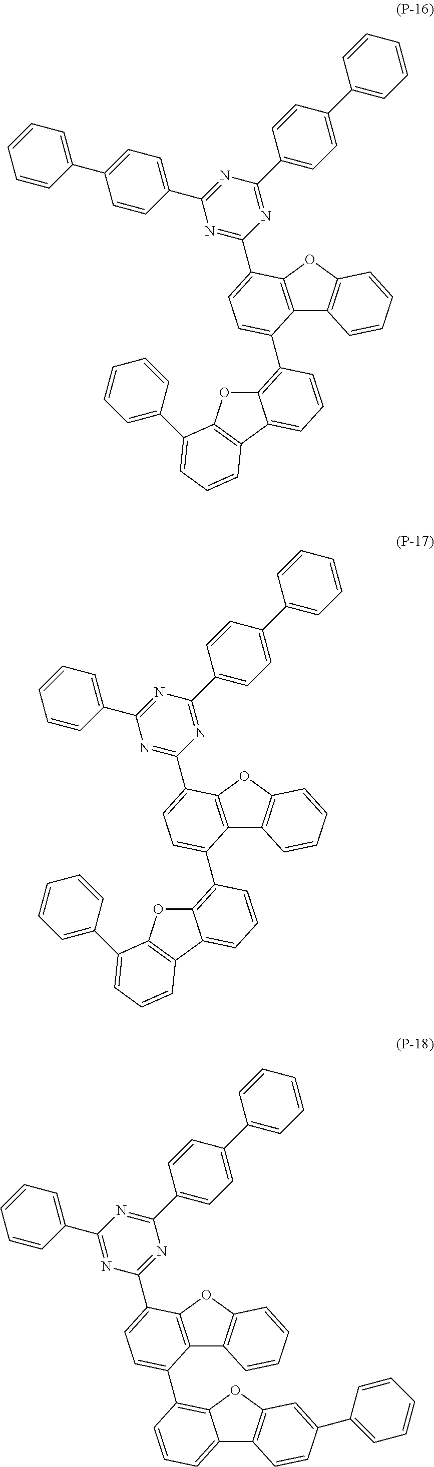 [SMILES: c1ccc(-c2ccc(-c3nc(-c4ccc(-c5ccccc5)cc4)nc(-c4ccc(-c5cccc6c5oc5c(-c7ccccc7)cccc56)c5c4oc4ccccc45)n3)cc2)cc1 ; c1ccc(-c2ccc(-c3nc(-c4ccccc4)nc(-c4ccc(-c5cccc6c5oc5c(-c7ccccc7)cccc56)c5c4oc4ccccc45)n3)cc2)cc1 ; c1ccc(-c2ccc(-c3nc(-c4ccccc4)nc(-c4ccc(-c5cccc6c5oc5cc(-c7ccccc7)ccc56)c5c4oc4ccccc45)n3)cc2)cc1]

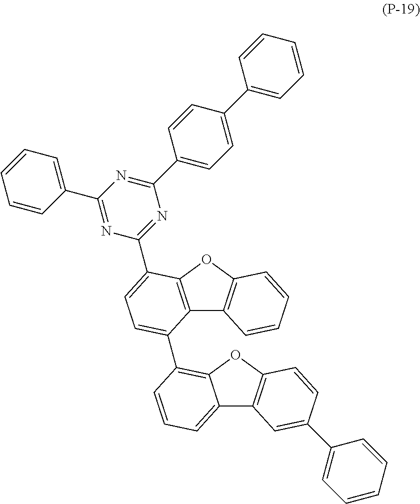 [SMILES: c1ccc(-c2ccc(-c3nc(-c4ccccc4)nc(-c4ccc(-c5cccc6c5oc5ccc(-c7ccccc7)cc56)c5c4oc4ccccc45)n3)cc2)cc1]

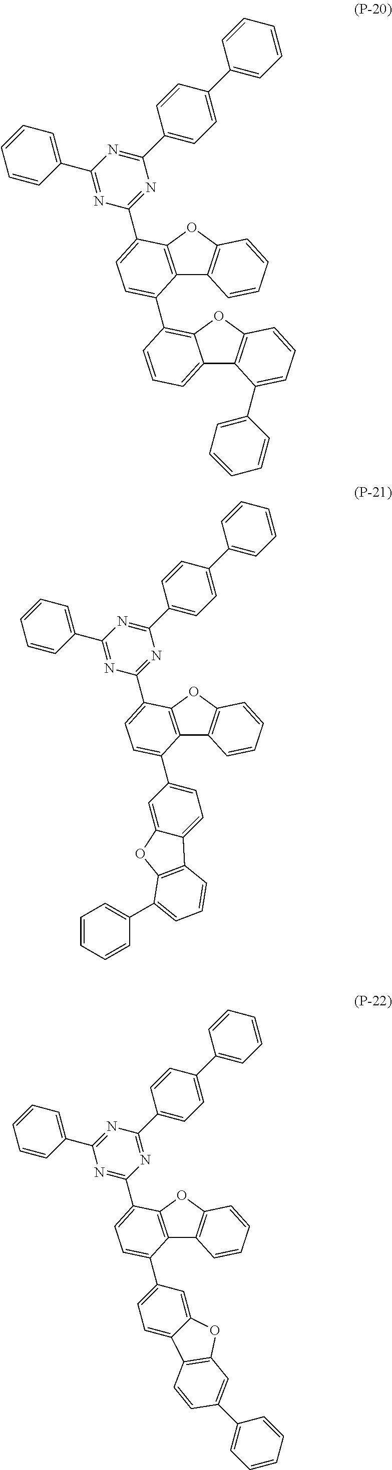

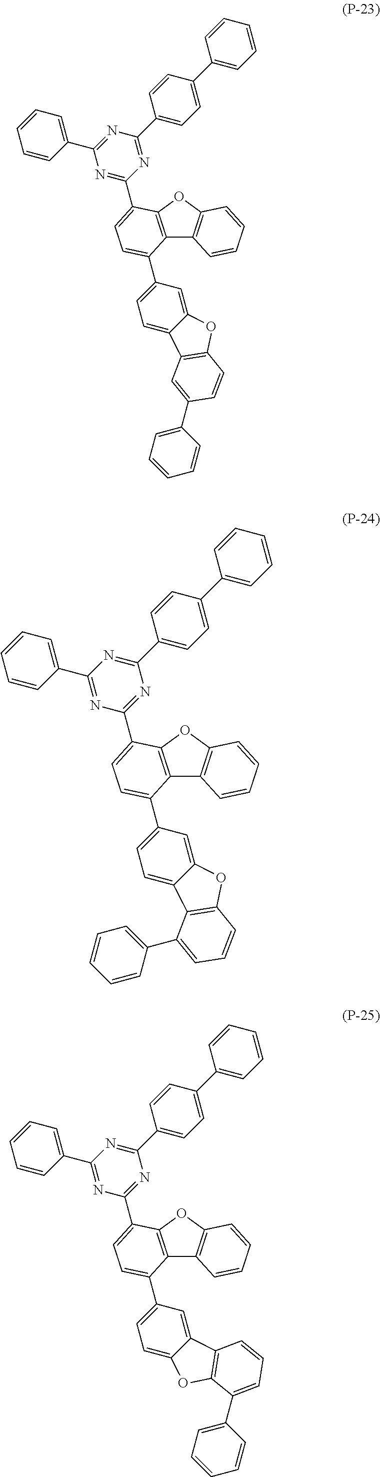 [SMILES: c1ccc(-c2ccc(-c3nc(-c4ccccc4)nc(-c4ccc(-c5ccc6c(c5)oc5ccc(-c7ccccc7)cc56)c5c4oc4ccccc45)n3)cc2)cc1 ; c1ccc(-c2ccc(-c3nc(-c4ccccc4)nc(-c4ccc(-c5ccc6c(c5)oc5cccc(-c7ccccc7)c56)c5c4oc4ccccc45)n3)cc2)cc1 ; c1ccc(-c2ccc(-c3nc(-c4ccccc4)nc(-c4ccc(-c5ccc6oc7c(-c8ccccc8)cccc7c6c5)c5c4oc4ccccc45)n3)cc2)cc1]

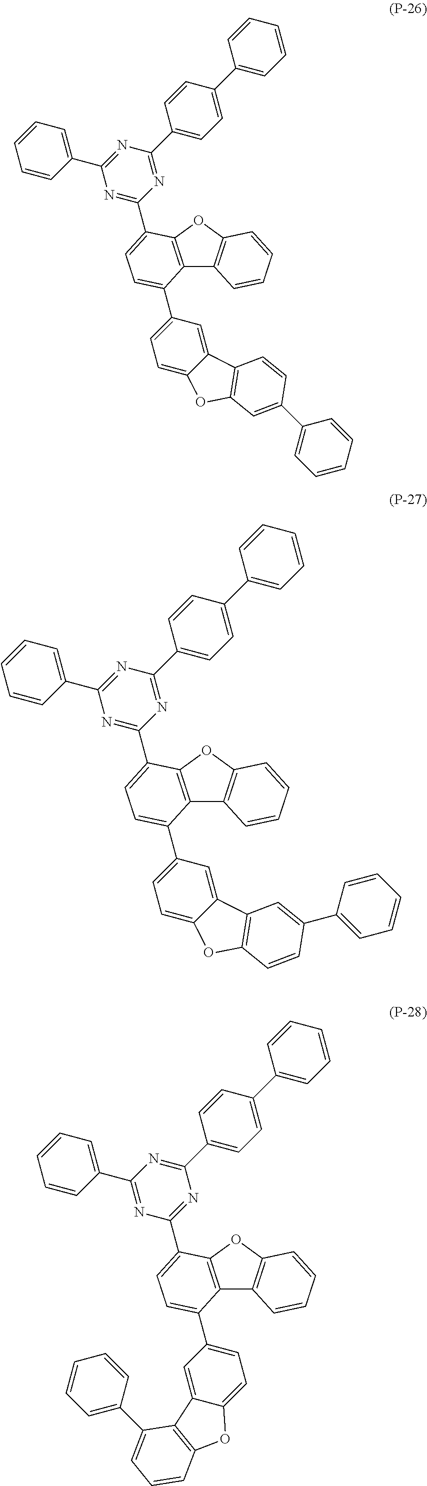 [SMILES: c1ccc(-c2ccc(-c3nc(-c4ccccc4)nc(-c4ccc(-c5ccc6oc7cc(-c8ccccc8)ccc7c6c5)c5c4oc4ccccc45)n3)cc2)cc1 ; c1ccc(-c2ccc(-c3nc(-c4ccccc4)nc(-c4ccc(-c5ccc6oc7ccc(-c8ccccc8)cc7c6c5)c5c4oc4ccccc45)n3)cc2)cc1 ; c1ccc(-c2ccc(-c3nc(-c4ccccc4)nc(-c4ccc(-c5ccc6oc7cccc(-c8ccccc8)c7c6c5)c5c4oc4ccccc45)n3)cc2)cc1]

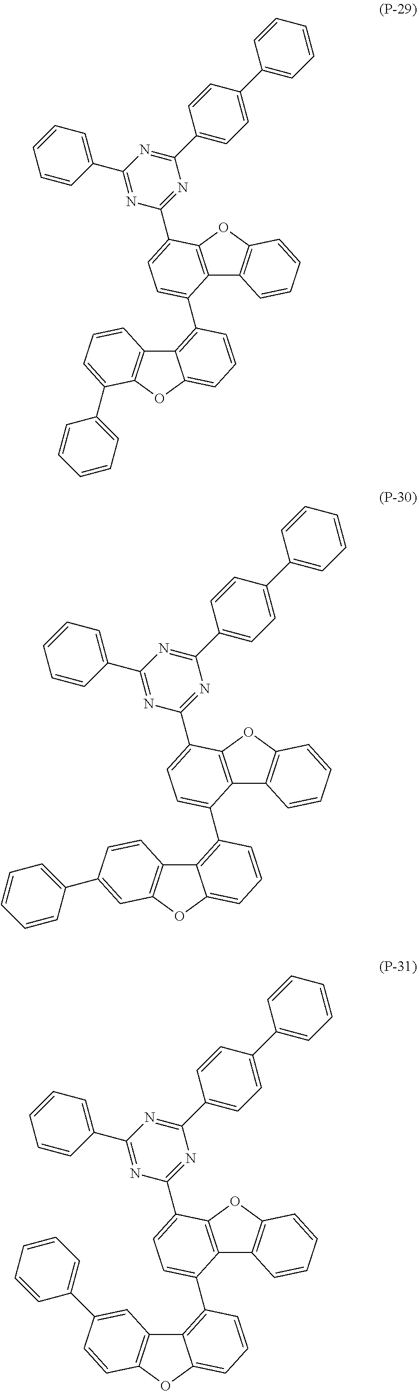 [SMILES: c1ccc(-c2ccc(-c3nc(-c4ccccc4)nc(-c4ccc(-c5cccc6oc7c(-c8ccccc8)cccc7c56)c5c4oc4ccccc45)n3)cc2)cc1 ; c1ccc(-c2ccc(-c3nc(-c4ccccc4)nc(-c4ccc(-c5cccc6oc7cc(-c8ccccc8)ccc7c56)c5c4oc4ccccc45)n3)cc2)cc1 ; c1ccc(-c2ccc(-c3nc(-c4ccccc4)nc(-c4ccc(-c5cccc6oc7ccc(-c8ccccc8)cc7c56)c5c4oc4ccccc45)n3)cc2)cc1]

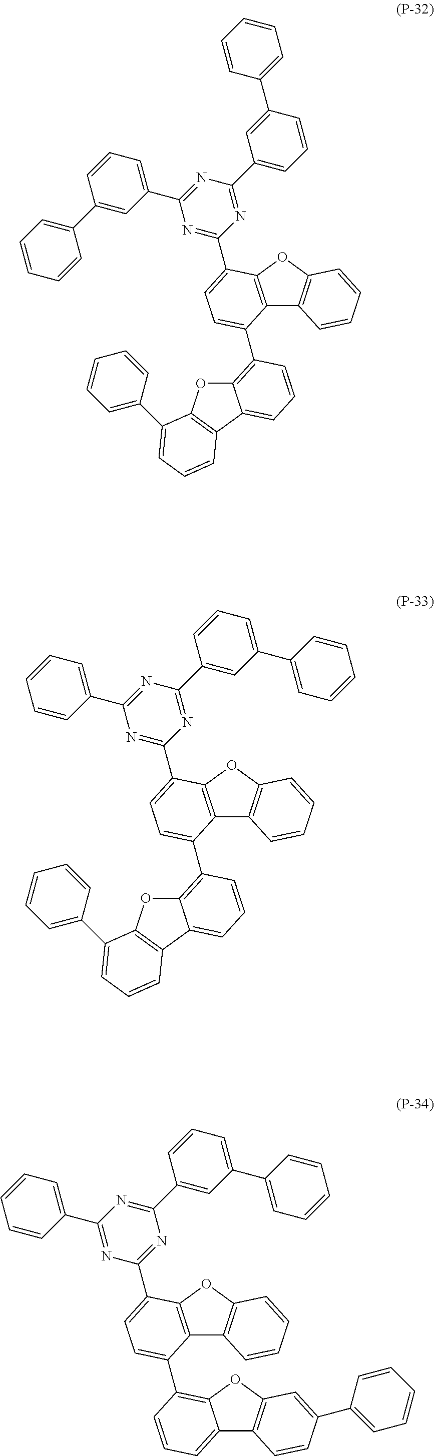 [SMILES: c1ccc(-c2cccc(-c3nc(-c4cccc(-c5ccccc5)c4)nc(-c4ccc(-c5cccc6c5oc5c(-c7ccccc7)cccc56)c5c4oc4ccccc45)n3)c2)cc1 ; c1ccc(-c2cccc(-c3nc(-c4ccccc4)nc(-c4ccc(-c5cccc6c5oc5c(-c7ccccc7)cccc56)c5c4oc4ccccc45)n3)c2)cc1 ; c1ccc(-c2cccc(-c3nc(-c4ccccc4)nc(-c4ccc(-c5cccc6c5oc5cc(-c7ccccc7)ccc56)c5c4oc4ccccc45)n3)c2)cc1]

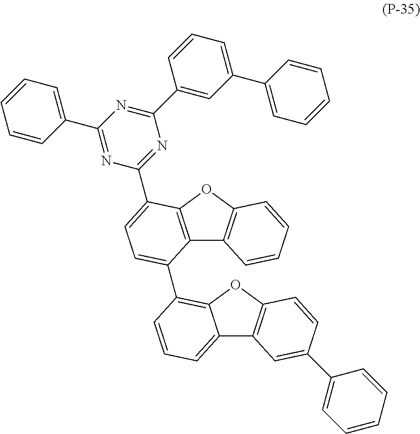 [SMILES: c1ccc(-c2cccc(-c3nc(-c4ccccc4)nc(-c4ccc(-c5cccc6c5oc5ccc(-c7ccccc7)cc56)c5c4oc4ccccc45)n3)c2)cc1]

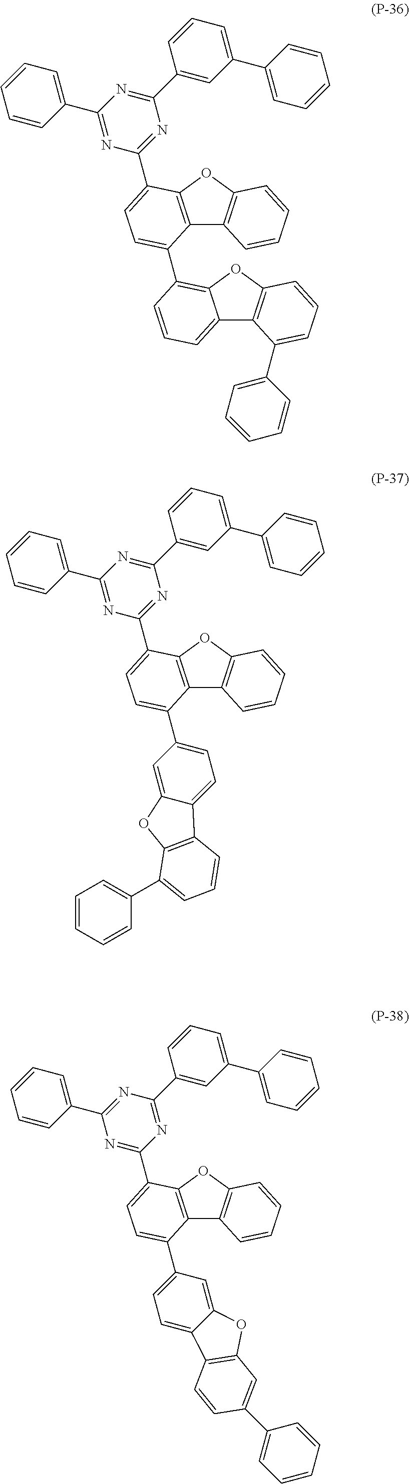 [SMILES: c1ccc(-c2cccc(-c3nc(-c4ccccc4)nc(-c4ccc(-c5cccc6c5oc5cccc(-c7ccccc7)c56)c5c4oc4ccccc45)n3)c2)cc1 ; c1ccc(-c2cccc(-c3nc(-c4ccccc4)nc(-c4ccc(-c5ccc6c(c5)oc5c(-c7ccccc7)cccc56)c5c4oc4ccccc45)n3)c2)cc1 ; c1ccc(-c2cccc(-c3nc(-c4ccccc4)nc(-c4ccc(-c5ccc6c(c5)oc5cc(-c7ccccc7)ccc56)c5c4oc4ccccc45)n3)c2)cc1]

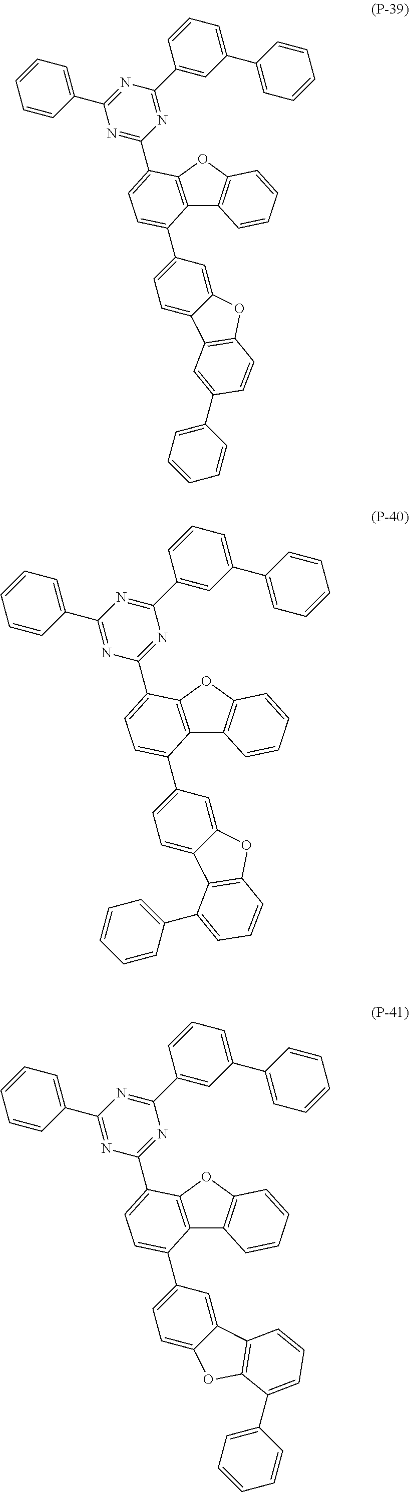

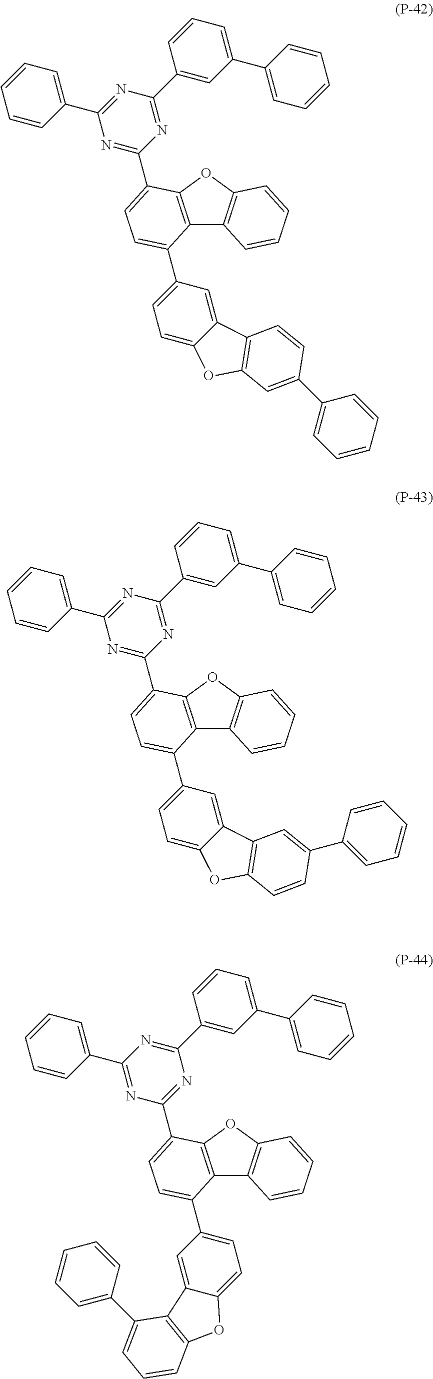 [SMILES: c1ccc(-c2cccc(-c3nc(-c4ccccc4)nc(-c4ccc(-c5ccc6oc7cc(-c8ccccc8)ccc7c6c5)c5c4oc4ccccc45)n3)c2)cc1 ; c1ccc(-c2cccc(-c3nc(-c4ccccc4)nc(-c4ccc(-c5ccc6oc7ccc(-c8ccccc8)cc7c6c5)c5c4oc4ccccc45)n3)c2)cc1 ; c1ccc(-c2cccc(-c3nc(-c4ccccc4)nc(-c4ccc(-c5ccc6oc7cccc(-c8ccccc8)c7c6c5)c5c4oc4ccccc45)n3)c2)cc1]

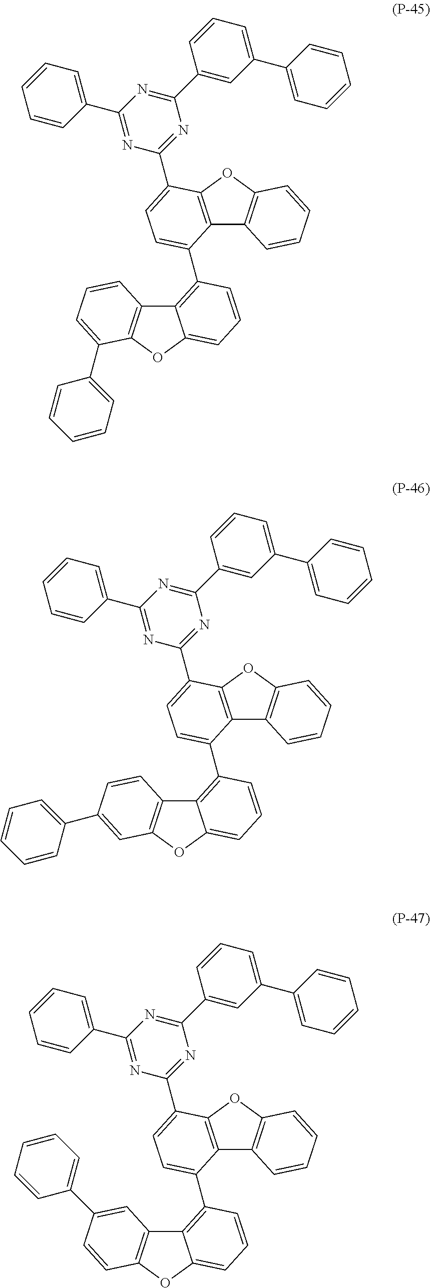 [SMILES: c1ccc(-c2cccc(-c3nc(-c4ccccc4)nc(-c4ccc(-c5cccc6oc7c(-c8ccccc8)cccc7c56)c5c4oc4ccccc45)n3)c2)cc1 ; c1ccc(-c2cccc(-c3nc(-c4ccccc4)nc(-c4ccc(-c5cccc6oc7cc(-c8ccccc8)ccc7c56)c5c4oc4ccccc45)n3)c2)cc1 ; c1ccc(-c2cccc(-c3nc(-c4ccccc4)nc(-c4ccc(-c5cccc6oc7ccc(-c8ccccc8)cc7c56)c5c4oc4ccccc45)n3)c2)cc1]

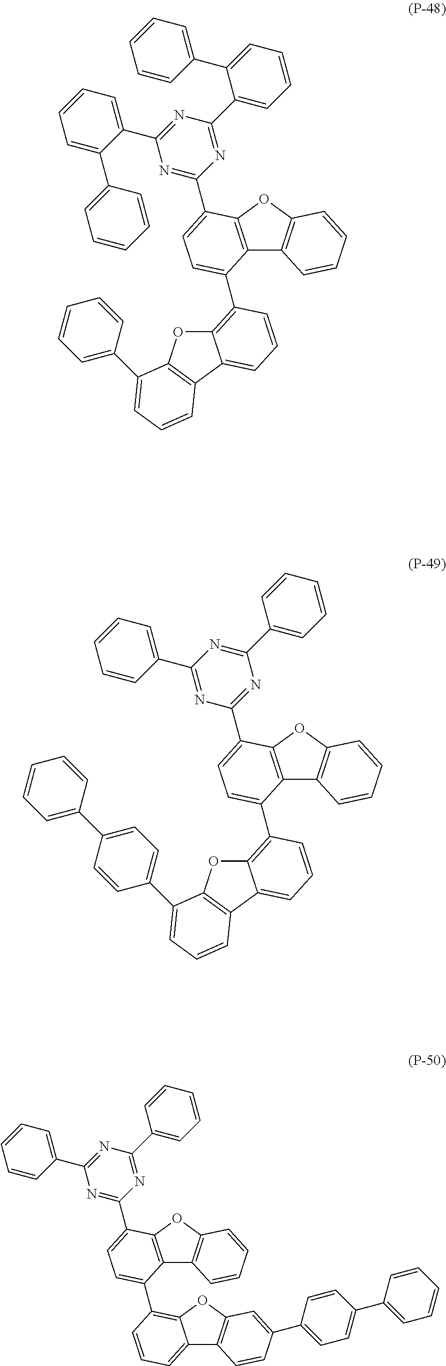 [SMILES: c1ccc(-c2ccccc2-c2nc(-c3ccccc3-c3ccccc3)nc(-c3ccc(-c4cccc5c4oc4c(-c6ccccc6)cccc45)c4c3oc3ccccc34)n2)cc1 ; c1ccc(-c2ccc(-c3cccc4c3oc3c(-c5ccc(-c6nc(-c7ccccc7)nc(-c7ccccc7)n6)c6oc7ccccc7c56)cccc34)cc2)cc1 ; c1ccc(-c2ccc(-c3ccc4c(c3)oc3c(-c5ccc(-c6nc(-c7ccccc7)nc(-c7ccccc7)n6)c6oc7ccccc7c56)cccc34)cc2)cc1]

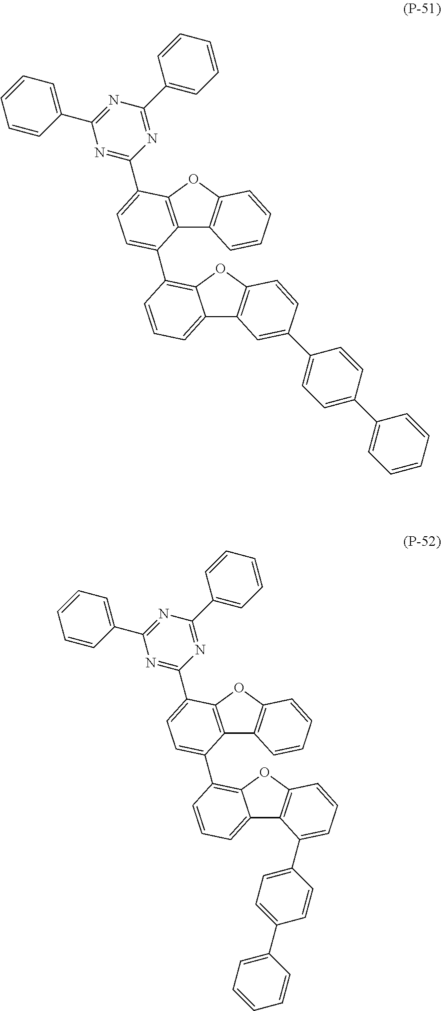 [SMILES: c1ccc(-c2ccc(-c3ccc4oc5c(-c6ccc(-c7nc(-c8ccccc8)nc(-c8ccccc8)n7)c7oc8ccccc8c67)cccc5c4c3)cc2)cc1 ; c1ccc(-c2ccc(-c3cccc4oc5c(-c6ccc(-c7nc(-c8ccccc8)nc(-c8ccccc8)n7)c7oc8ccccc8c67)cccc5c34)cc2)cc1]

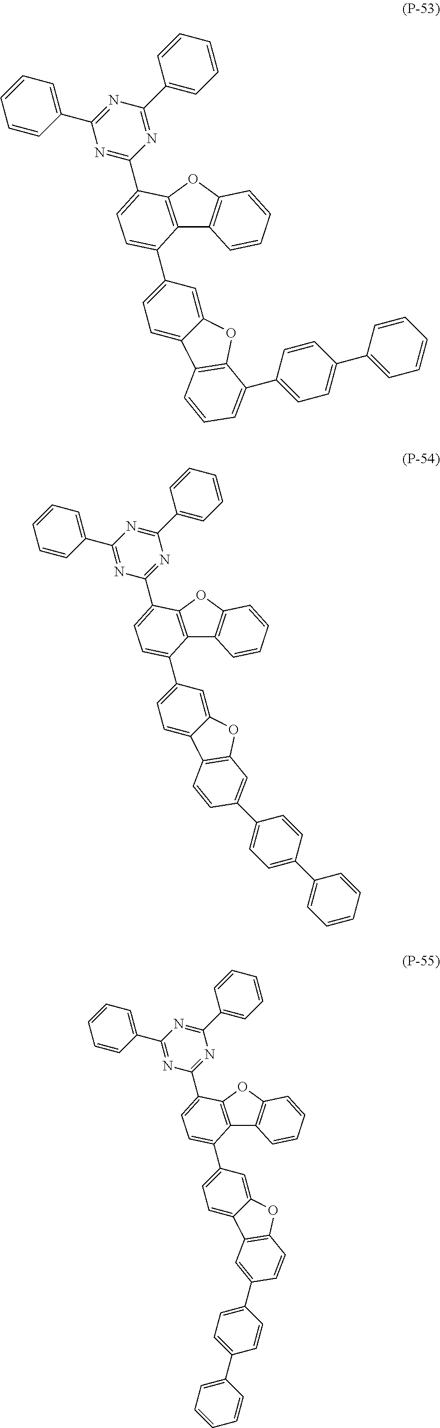 [SMILES: c1ccc(-c2ccc(-c3cccc4c3oc3cc(-c5ccc(-c6nc(-c7ccccc7)nc(-c7ccccc7)n6)c6oc7ccccc7c56)ccc34)cc2)cc1 ; c1ccc(-c2ccc(-c3ccc4c(c3)oc3cc(-c5ccc(-c6nc(-c7ccccc7)nc(-c7ccccc7)n6)c6oc7ccccc7c56)ccc34)cc2)cc1 ; c1ccc(-c2ccc(-c3ccc4oc5cc(-c6ccc(-c7nc(-c8ccccc8)nc(-c8ccccc8)n7)c7oc8ccccc8c67)ccc5c4c3)cc2)cc1]

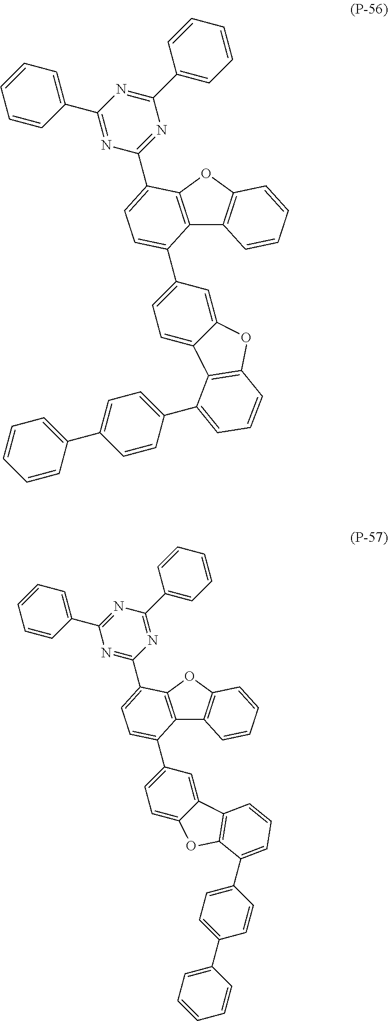 [SMILES: c1ccc(-c2ccc(-c3cccc4oc5cc(-c6ccc(-c7nc(-c8ccccc8)nc(-c8ccccc8)n7)c7oc8ccccc8c67)ccc5c34)cc2)cc1 ; c1ccc(-c2ccc(-c3cccc4c3oc3ccc(-c5ccc(-c6nc(-c7ccccc7)nc(-c7ccccc7)n6)c6oc7ccccc7c56)cc34)cc2)cc1]

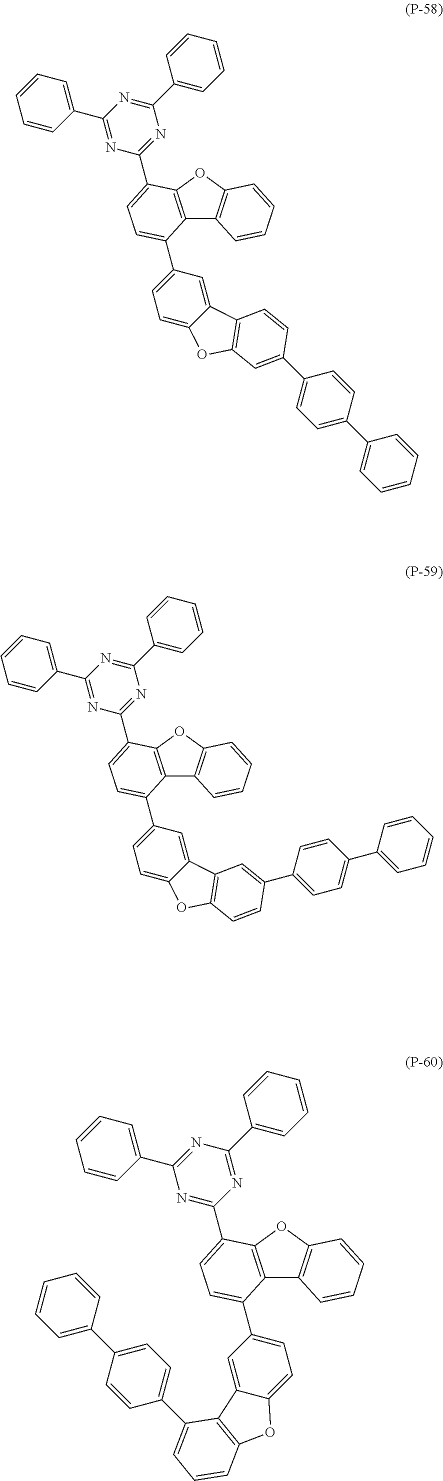 [SMILES: c1ccc(-c2ccc(-c3ccc4c(c3)oc3ccc(-c5ccc(-c6nc(-c7ccccc7)nc(-c7ccccc7)n6)c6oc7ccccc7c56)cc34)cc2)cc1 ; c1ccc(-c2ccc(-c3ccc4oc5ccc(-c6ccc(-c7nc(-c8ccccc8)nc(-c8ccccc8)n7)c7oc8ccccc8c67)cc5c4c3)cc2)cc1 ; c1ccc(-c2ccc(-c3cccc4oc5ccc(-c6ccc(-c7nc(-c8ccccc8)nc(-c8ccccc8)n7)c7oc8ccccc8c67)cc5c34)cc2)cc1]

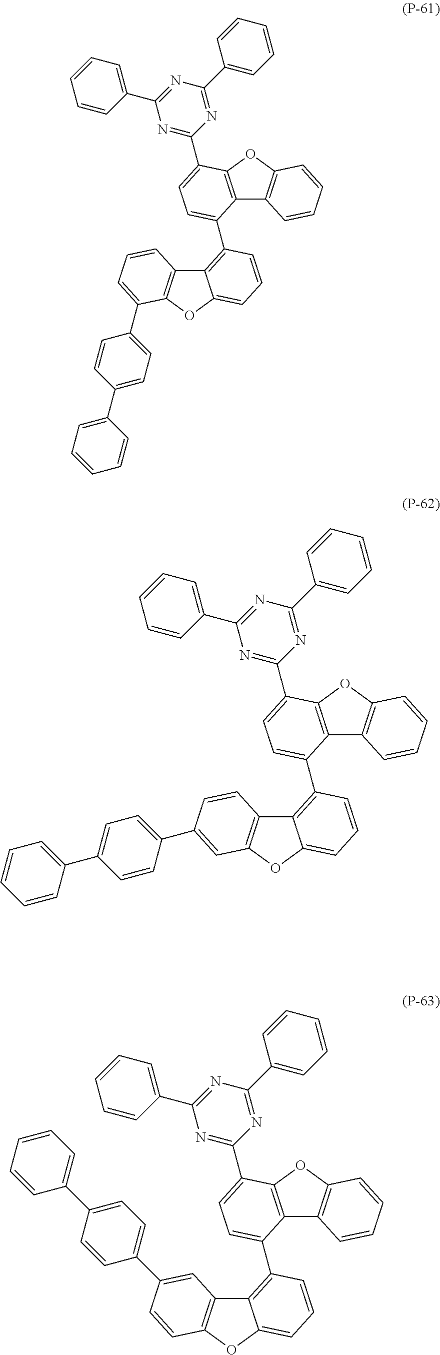 [SMILES: c1ccc(-c2ccc(-c3cccc4c3oc3cccc(-c5ccc(-c6nc(-c7ccccc7)nc(-c7ccccc7)n6)c6oc7ccccc7c56)c34)cc2)cc1 ; c1ccc(-c2ccc(-c3ccc4c(c3)oc3cccc(-c5ccc(-c6nc(-c7ccccc7)nc(-c7ccccc7)n6)c6oc7ccccc7c56)c34)cc2)cc1 ; c1ccc(-c2ccc(-c3ccc4oc5cccc(-c6ccc(-c7nc(-c8ccccc8)nc(-c8ccccc8)n7)c7oc8ccccc8c67)c5c4c3)cc2)cc1]

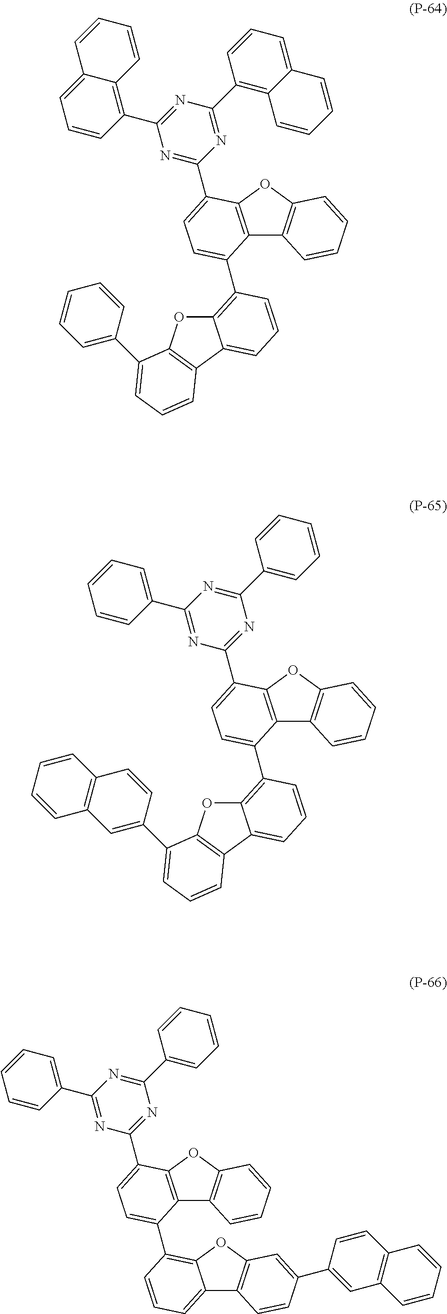 [SMILES: c1ccc(-c2cccc3c2oc2c(-c4ccc(-c5nc(-c6cccc7ccccc67)nc(-c6cccc7ccccc67)n5)c5oc6ccccc6c45)cccc23)cc1 ; c1ccc(-c2nc(-c3ccccc3)nc(-c3ccc(-c4cccc5c4oc4c(-c6ccc7ccccc7c6)cccc45)c4c3oc3ccccc34)n2)cc1 ; c1ccc(-c2nc(-c3ccccc3)nc(-c3ccc(-c4cccc5c4oc4cc(-c6ccc7ccccc7c6)ccc45)c4c3oc3ccccc34)n2)cc1]

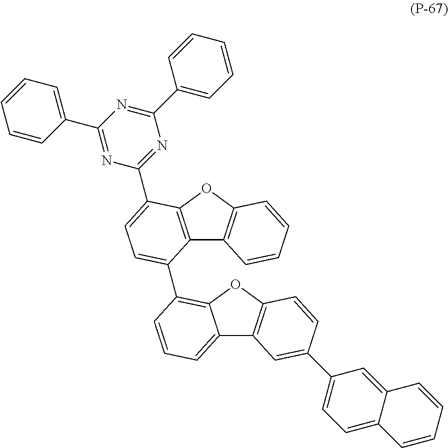 [SMILES: c1ccc(-c2nc(-c3ccccc3)nc(-c3ccc(-c4cccc5c4oc4ccc(-c6ccc7ccccc7c6)cc45)c4c3oc3ccccc34)n2)cc1]

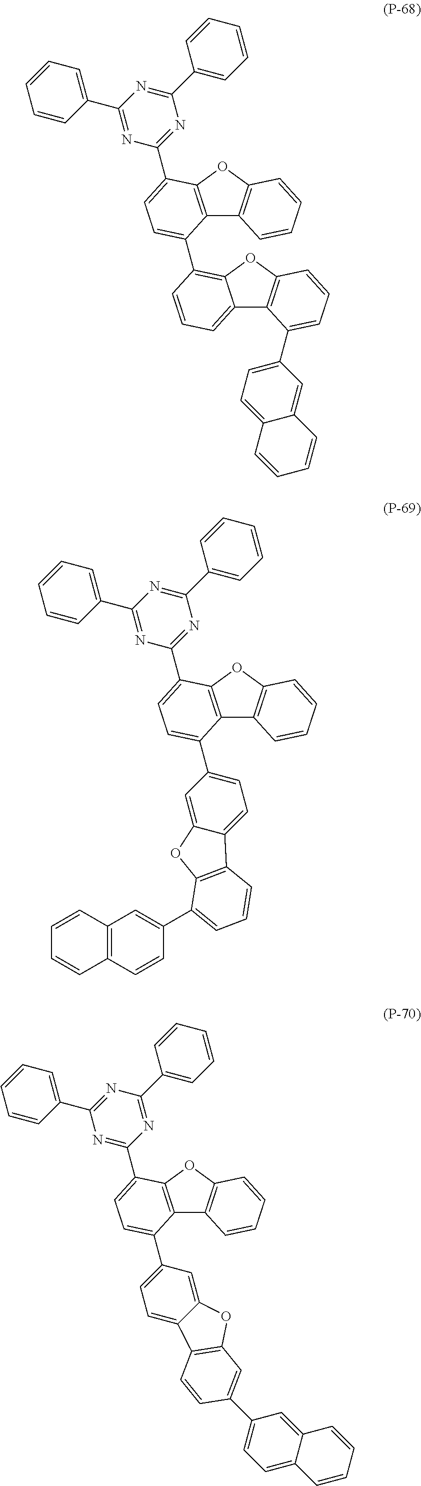 [SMILES: c1ccc(-c2nc(-c3ccccc3)nc(-c3ccc(-c4cccc5c4oc4cccc(-c6ccc7ccccc7c6)c45)c4c3oc3ccccc34)n2)cc1 ; c1ccc(-c2nc(-c3ccccc3)nc(-c3ccc(-c4ccc5c(c4)oc4c(-c6ccc7ccccc7c6)cccc45)c4c3oc3ccccc34)n2)cc1 ; c1ccc(-c2nc(-c3ccccc3)nc(-c3ccc(-c4ccc5c(c4)oc4cc(-c6ccc7ccccc7c6)ccc45)c4c3oc3ccccc34)n2)cc1]

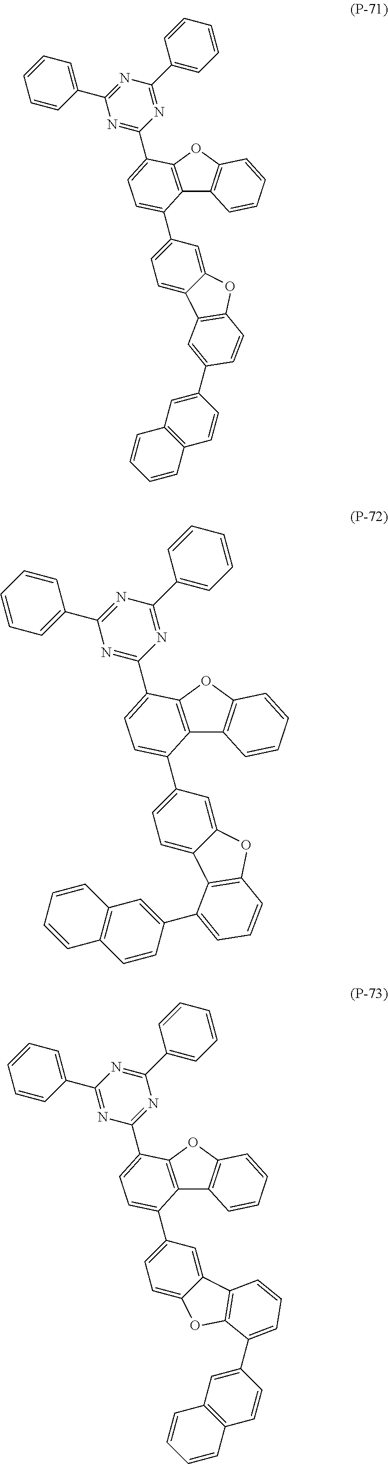 [SMILES: c1ccc(-c2nc(-c3ccccc3)nc(-c3ccc(-c4ccc5c(c4)oc4ccc(-c6ccc7ccccc7c6)cc45)c4c3oc3ccccc34)n2)cc1 ; c1ccc(-c2nc(-c3ccccc3)nc(-c3ccc(-c4ccc5c(c4)oc4cccc(-c6ccc7ccccc7c6)c45)c4c3oc3ccccc34)n2)cc1 ; c1ccc(-c2nc(-c3ccccc3)nc(-c3ccc(-c4ccc5oc6c(-c7ccc8ccccc8c7)cccc6c5c4)c4c3oc3ccccc34)n2)cc1]

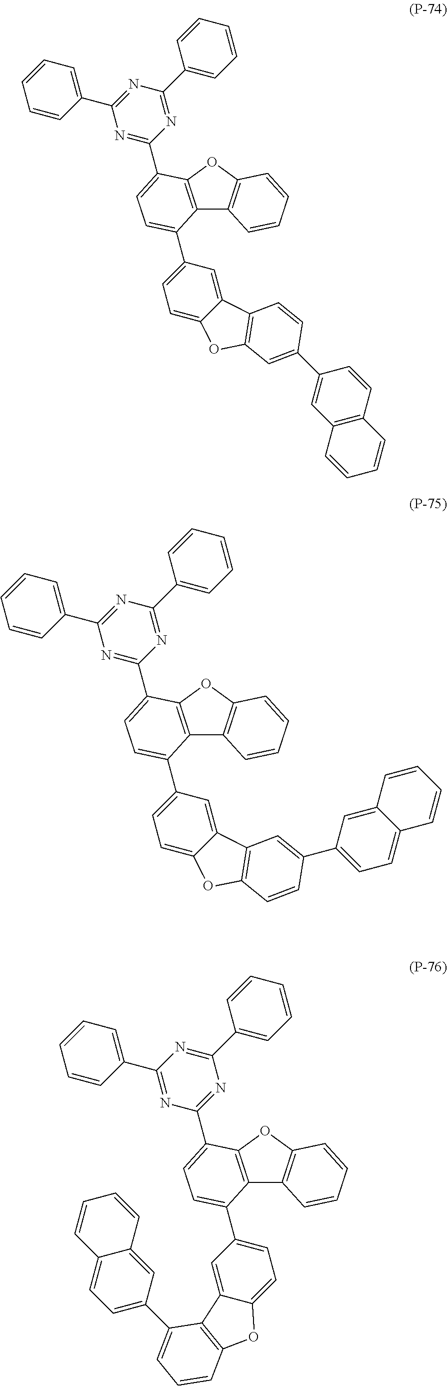 [SMILES: c1ccc(-c2nc(-c3ccccc3)nc(-c3ccc(-c4ccc5oc6cc(-c7ccc8ccccc8c7)ccc6c5c4)c4c3oc3ccccc34)n2)cc1 ; c1ccc(-c2nc(-c3ccccc3)nc(-c3ccc(-c4ccc5oc6ccc(-c7ccc8ccccc8c7)cc6c5c4)c4c3oc3ccccc34)n2)cc1 ; c1ccc(-c2nc(-c3ccccc3)nc(-c3ccc(-c4ccc5oc6cccc(-c7ccc8ccccc8c7)c6c5c4)c4c3oc3ccccc34)n2)cc1]

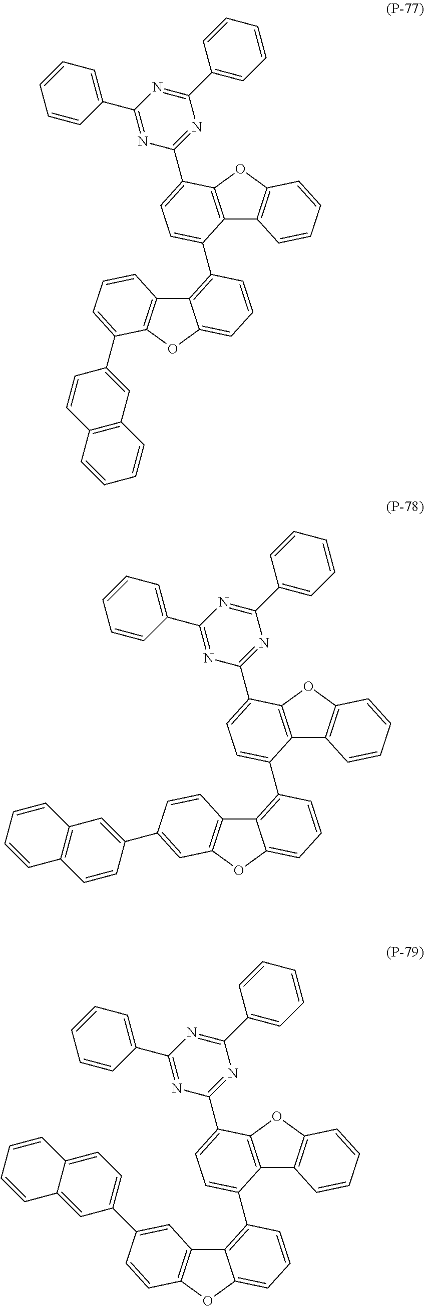 [SMILES: c1ccc(-c2nc(-c3ccccc3)nc(-c3ccc(-c4cccc5oc6c(-c7ccc8ccccc8c7)cccc6c45)c4c3oc3ccccc34)n2)cc1 ; c1ccc(-c2nc(-c3ccccc3)nc(-c3ccc(-c4cccc5oc6cc(-c7ccc8ccccc8c7)ccc6c45)c4c3oc3ccccc34)n2)cc1 ; c1ccc(-c2nc(-c3ccccc3)nc(-c3ccc(-c4cccc5oc6ccc(-c7ccc8ccccc8c7)cc6c45)c4c3oc3ccccc34)n2)cc1]

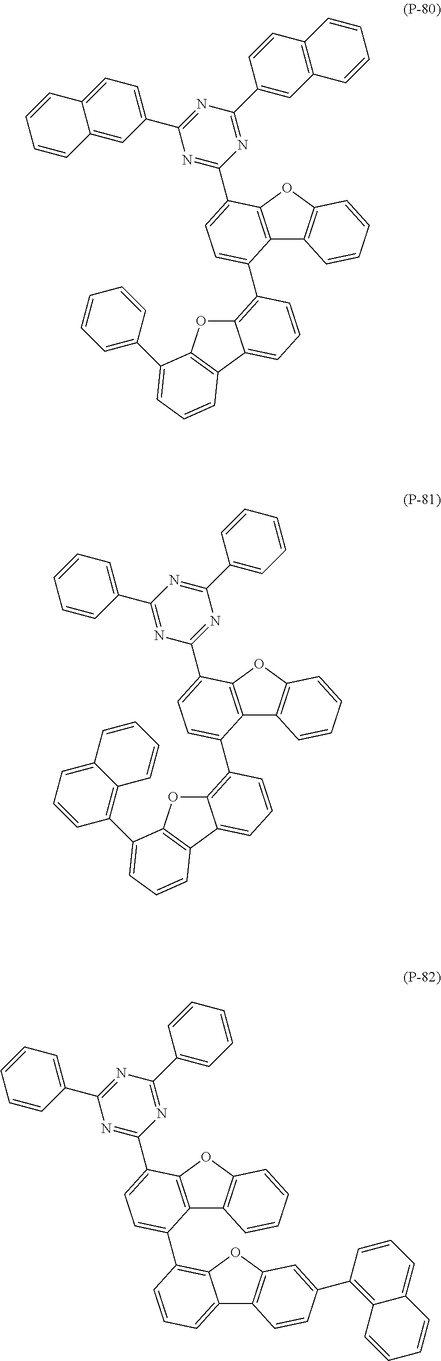 [SMILES: c1ccc(-c2cccc3c2oc2c(-c4ccc(-c5nc(-c6ccc7ccccc7c6)nc(-c6ccc7ccccc7c6)n5)c5oc6ccccc6c45)cccc23)cc1 ; c1ccc(-c2nc(-c3ccccc3)nc(-c3ccc(-c4cccc5c4oc4c(-c6cccc7ccccc67)cccc45)c4c3oc3ccccc34)n2)cc1 ; c1ccc(-c2nc(-c3ccccc3)nc(-c3ccc(-c4cccc5c4oc4cc(-c6cccc7ccccc67)ccc45)c4c3oc3ccccc34)n2)cc1]

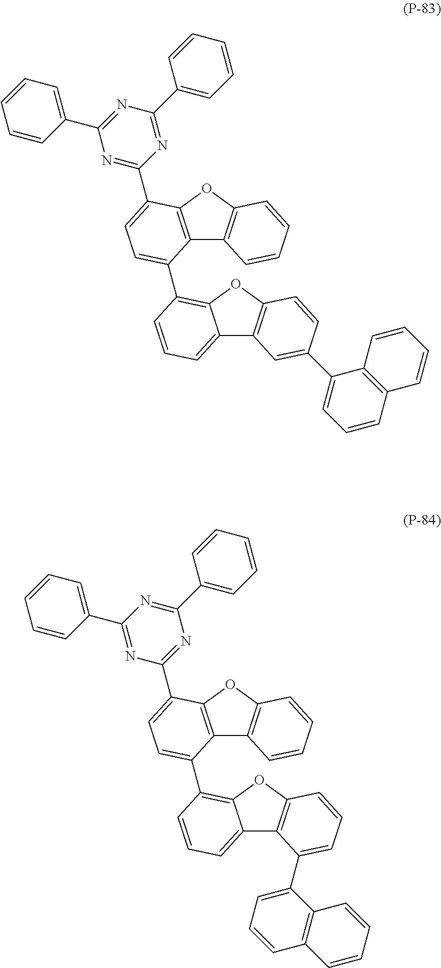 [SMILES: c1ccc(-c2nc(-c3ccccc3)nc(-c3ccc(-c4cccc5c4oc4ccc(-c6cccc7ccccc67)cc45)c4c3oc3ccccc34)n2)cc1 ; c1ccc(-c2nc(-c3ccccc3)nc(-c3ccc(-c4cccc5c4oc4cccc(-c6cccc7ccccc67)c45)c4c3oc3ccccc34)n2)cc1]

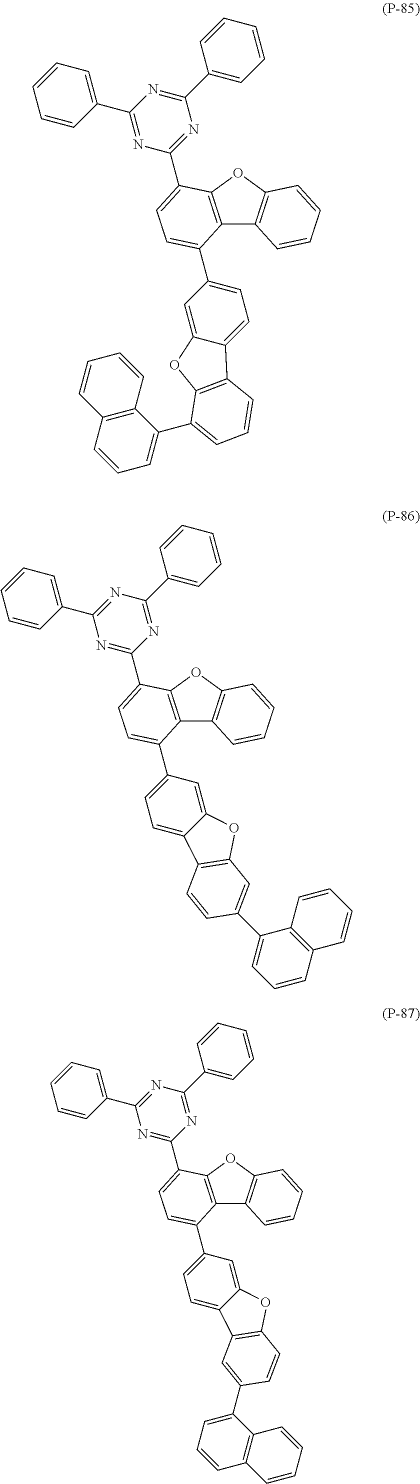 [SMILES: c1ccc(-c2nc(-c3ccccc3)nc(-c3ccc(-c4ccc5c(c4)oc4c(-c6cccc7ccccc67)cccc45)c4c3oc3ccccc34)n2)cc1 ; c1ccc(-c2nc(-c3ccccc3)nc(-c3ccc(-c4ccc5c(c4)oc4cc(-c6cccc7ccccc67)ccc45)c4c3oc3ccccc34)n2)cc1 ; c1ccc(-c2nc(-c3ccccc3)nc(-c3ccc(-c4ccc5c(c4)oc4ccc(-c6cccc7ccccc67)cc45)c4c3oc3ccccc34)n2)cc1]

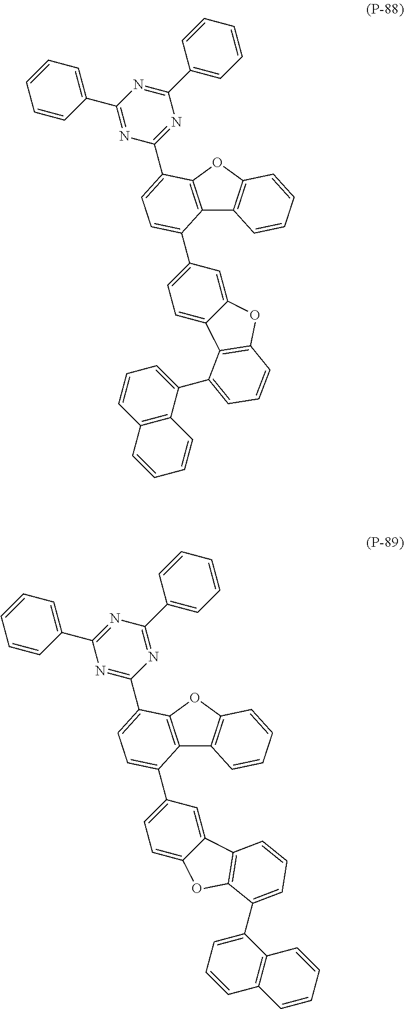 [SMILES: c1ccc(-c2nc(-c3ccccc3)nc(-c3ccc(-c4ccc5c(c4)oc4cccc(-c6cccc7ccccc67)c45)c4c3oc3ccccc34)n2)cc1 ; c1ccc(-c2nc(-c3ccccc3)nc(-c3ccc(-c4ccc5oc6c(-c7cccc8ccccc78)cccc6c5c4)c4c3oc3ccccc34)n2)cc1]

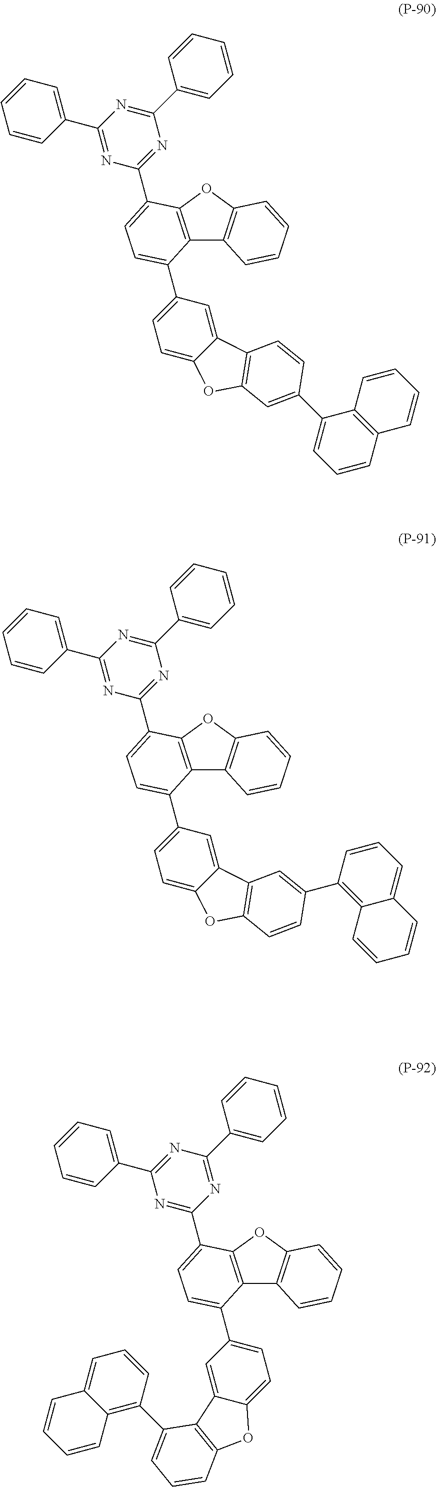 [SMILES: c1ccc(-c2nc(-c3ccccc3)nc(-c3ccc(-c4ccc5oc6cc(-c7cccc8ccccc78)ccc6c5c4)c4c3oc3ccccc34)n2)cc1 ; c1ccc(-c2nc(-c3ccccc3)nc(-c3ccc(-c4ccc5oc6ccc(-c7cccc8ccccc78)cc6c5c4)c4c3oc3ccccc34)n2)cc1 ; c1ccc(-c2nc(-c3ccccc3)nc(-c3ccc(-c4ccc5oc6cccc(-c7cccc8ccccc78)c6c5c4)c4c3oc3ccccc34)n2)cc1]

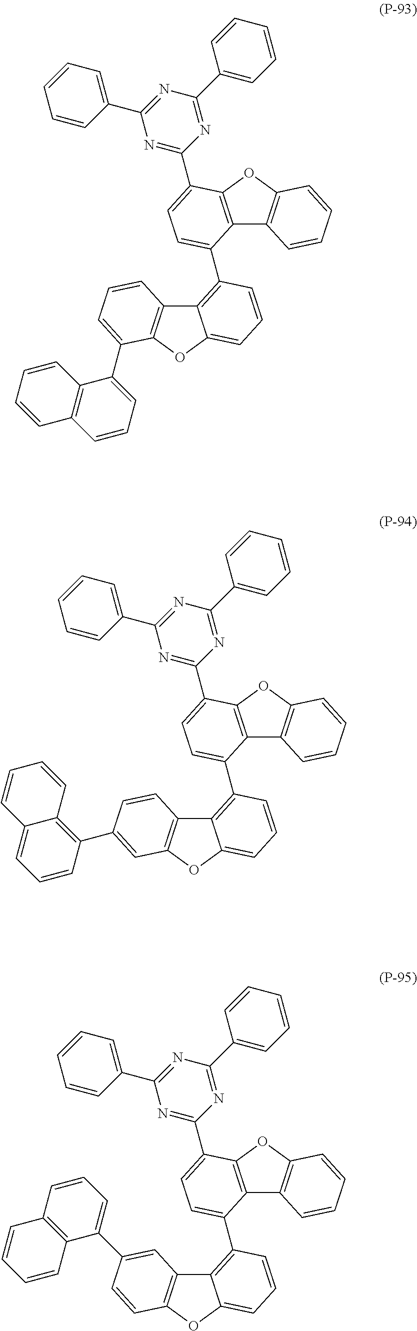 [SMILES: c1ccc(-c2nc(-c3ccccc3)nc(-c3ccc(-c4cccc5oc6c(-c7cccc8ccccc78)cccc6c45)c4c3oc3ccccc34)n2)cc1 ; c1ccc(-c2nc(-c3ccccc3)nc(-c3ccc(-c4cccc5oc6cc(-c7cccc8ccccc78)ccc6c45)c4c3oc3ccccc34)n2)cc1 ; c1ccc(-c2nc(-c3ccccc3)nc(-c3ccc(-c4cccc5oc6ccc(-c7cccc8ccccc78)cc6c45)c4c3oc3ccccc34)n2)cc1]

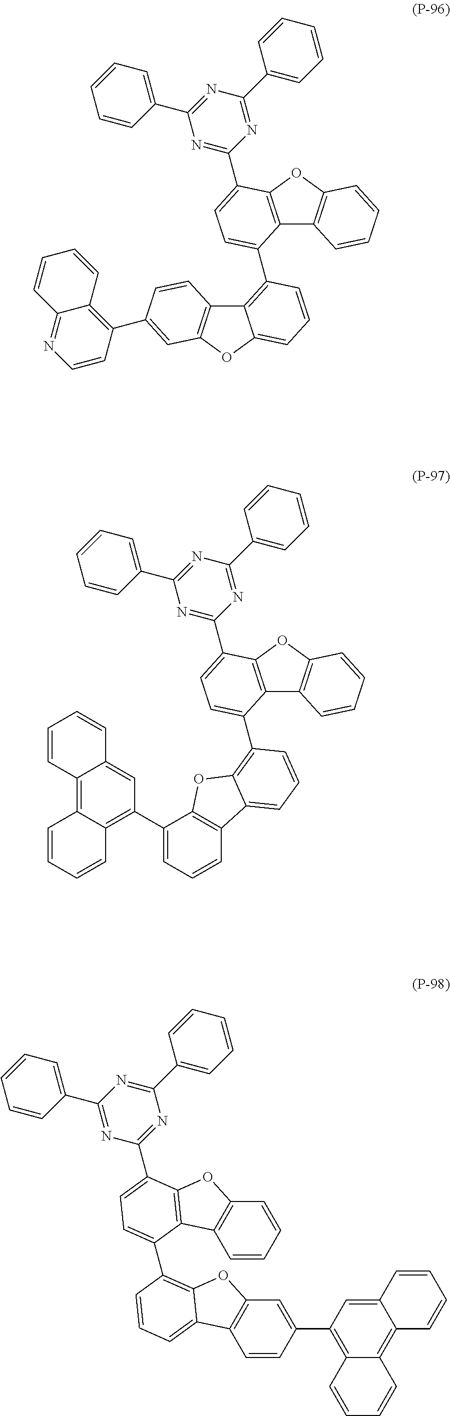 [SMILES: c1ccc(-c2nc(-c3ccccc3)nc(-c3ccc(-c4cccc5oc6cc(-c7ccnc8ccccc78)ccc6c45)c4c3oc3ccccc34)n2)cc1 ; c1ccc(-c2nc(-c3ccccc3)nc(-c3ccc(-c4cccc5c4oc4c(-c6cc7ccccc7c7ccccc67)cccc45)c4c3oc3ccccc34)n2)cc1 ; c1ccc(-c2nc(-c3ccccc3)nc(-c3ccc(-c4cccc5c4oc4cc(-c6cc7ccccc7c7ccccc67)ccc45)c4c3oc3ccccc34)n2)cc1]

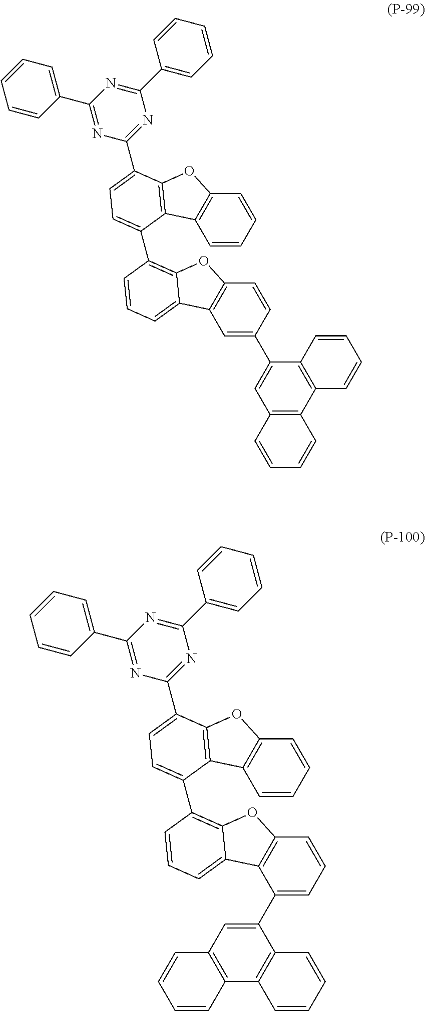 [SMILES: c1ccc(-c2nc(-c3ccccc3)nc(-c3ccc(-c4cccc5c4oc4ccc(-c6cc7ccccc7c7ccccc67)cc45)c4c3oc3ccccc34)n2)cc1 ; c1ccc(-c2nc(-c3ccccc3)nc(-c3ccc(-c4cccc5c4oc4cccc(-c6cc7ccccc7c7ccccc67)c45)c4c3oc3ccccc34)n2)cc1]

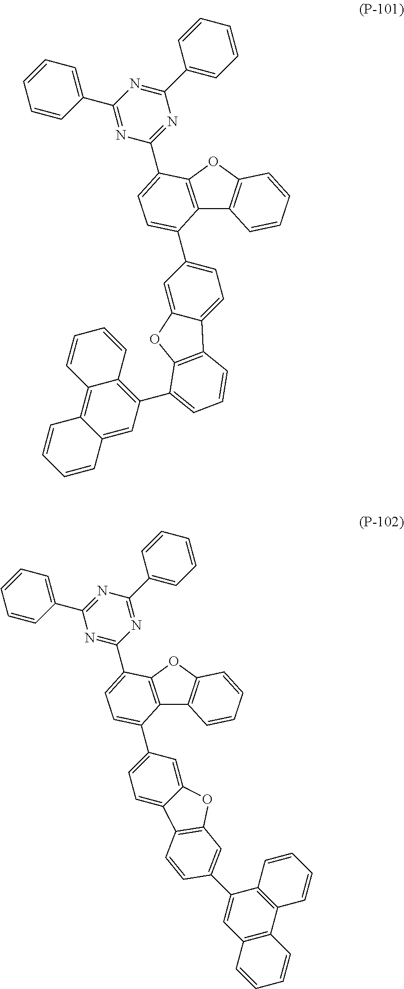 [SMILES: c1ccc(-c2nc(-c3ccccc3)nc(-c3ccc(-c4ccc5c(c4)oc4c(-c6cc7ccccc7c7ccccc67)cccc45)c4c3oc3ccccc34)n2)cc1 ; c1ccc(-c2nc(-c3ccccc3)nc(-c3ccc(-c4ccc5c(c4)oc4cc(-c6cc7ccccc7c7ccccc67)ccc45)c4c3oc3ccccc34)n2)cc1]

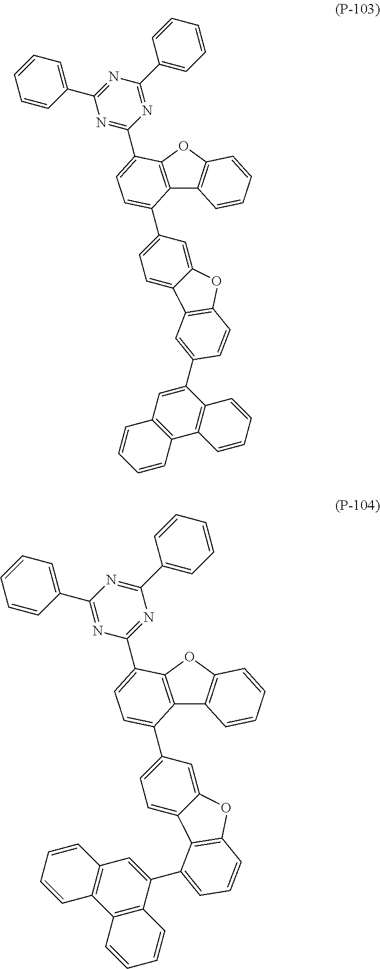 [SMILES: c1ccc(-c2nc(-c3ccccc3)nc(-c3ccc(-c4ccc5c(c4)oc4ccc(-c6cc7ccccc7c7ccccc67)cc45)c4c3oc3ccccc34)n2)cc1 ; c1ccc(-c2nc(-c3ccccc3)nc(-c3ccc(-c4ccc5c(c4)oc4cccc(-c6cc7ccccc7c7ccccc67)c45)c4c3oc3ccccc34)n2)cc1]

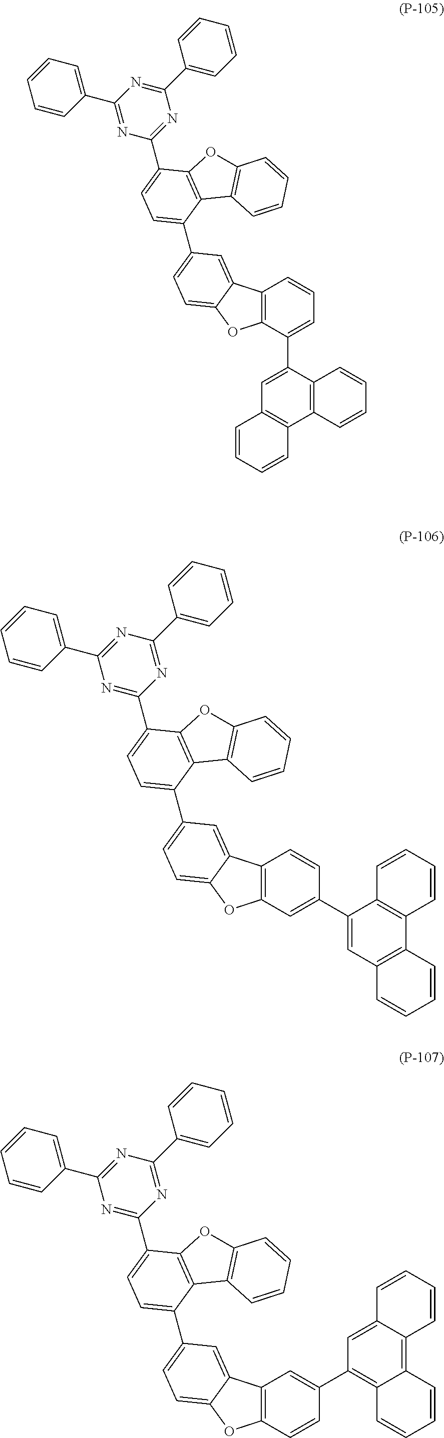 [SMILES: c1ccc(-c2nc(-c3ccccc3)nc(-c3ccc(-c4ccc5oc6c(-c7cc8ccccc8c8ccccc78)cccc6c5c4)c4c3oc3ccccc34)n2)cc1 ; c1ccc(-c2nc(-c3ccccc3)nc(-c3ccc(-c4ccc5oc6cc(-c7cc8ccccc8c8ccccc78)ccc6c5c4)c4c3oc3ccccc34)n2)cc1 ; c1ccc(-c2nc(-c3ccccc3)nc(-c3ccc(-c4ccc5oc6ccc(-c7cc8ccccc8c8ccccc78)cc6c5c4)c4c3oc3ccccc34)n2)cc1]

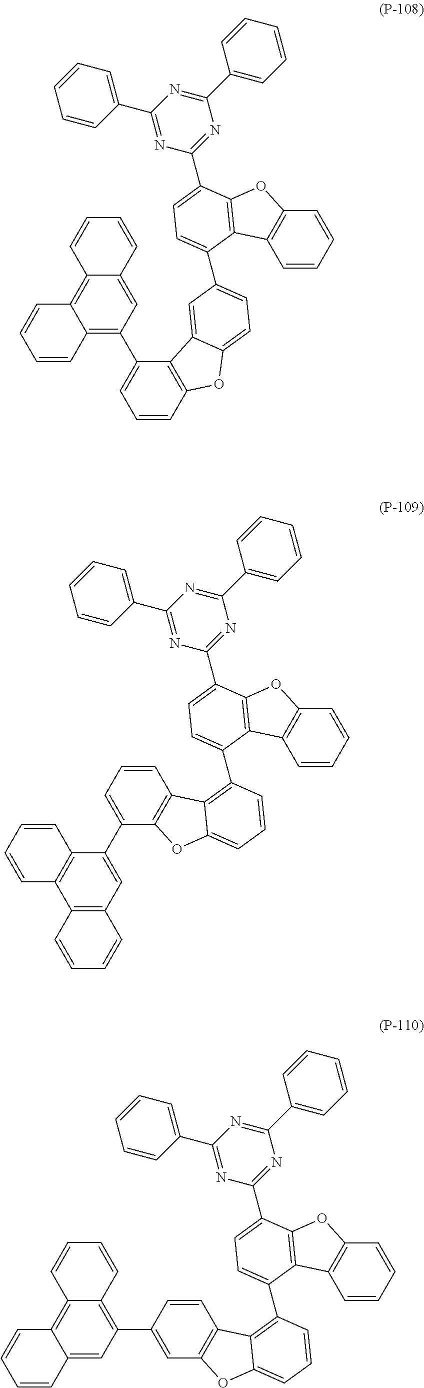 [SMILES: c1ccc(-c2nc(-c3ccccc3)nc(-c3ccc(-c4ccc5oc6cccc(-c7cc8ccccc8c8ccccc78)c6c5c4)c4c3oc3ccccc34)n2)cc1 ; c1ccc(-c2nc(-c3ccccc3)nc(-c3ccc(-c4cccc5oc6c(-c7cc8ccccc8c8ccccc78)cccc6c45)c4c3oc3ccccc34)n2)cc1 ; c1ccc(-c2nc(-c3ccccc3)nc(-c3ccc(-c4cccc5oc6cc(-c7cc8ccccc8c8ccccc78)ccc6c45)c4c3oc3ccccc34)n2)cc1]

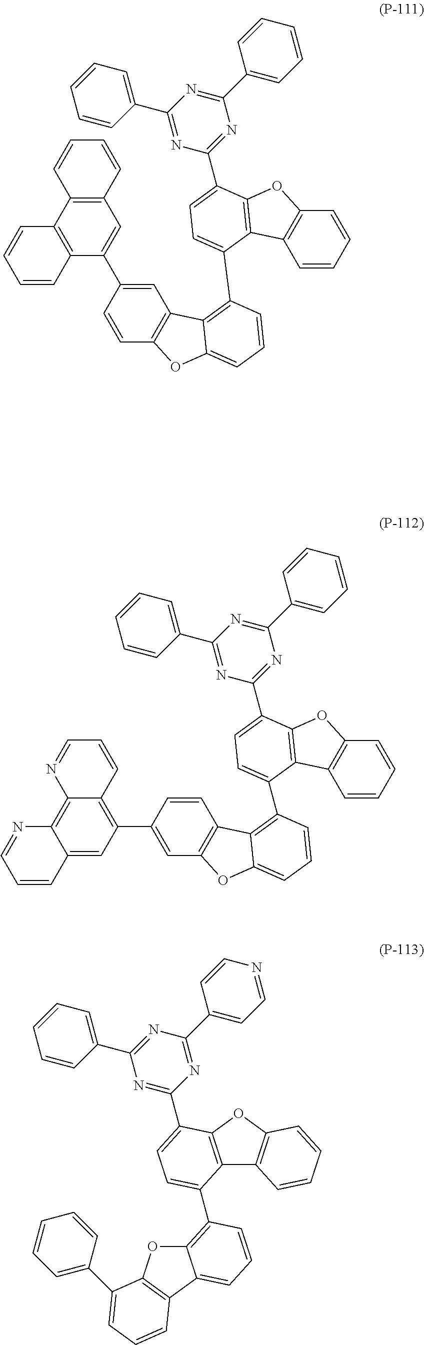 [SMILES: c1ccc(-c2nc(-c3ccccc3)nc(-c3ccc(-c4cccc5oc6ccc(-c7cc8ccccc8c8ccccc78)cc6c45)c4c3oc3ccccc34)n2)cc1 ; c1ccc(-c2nc(-c3ccccc3)nc(-c3ccc(-c4cccc5oc6cc(-c7cc8cccnc8c8ncccc78)ccc6c45)c4c3oc3ccccc34)n2)cc1 ; c1ccc(-c2nc(-c3ccncc3)nc(-c3ccc(-c4cccc5c4oc4c(-c6ccccc6)cccc45)c4c3oc3ccccc34)n2)cc1]

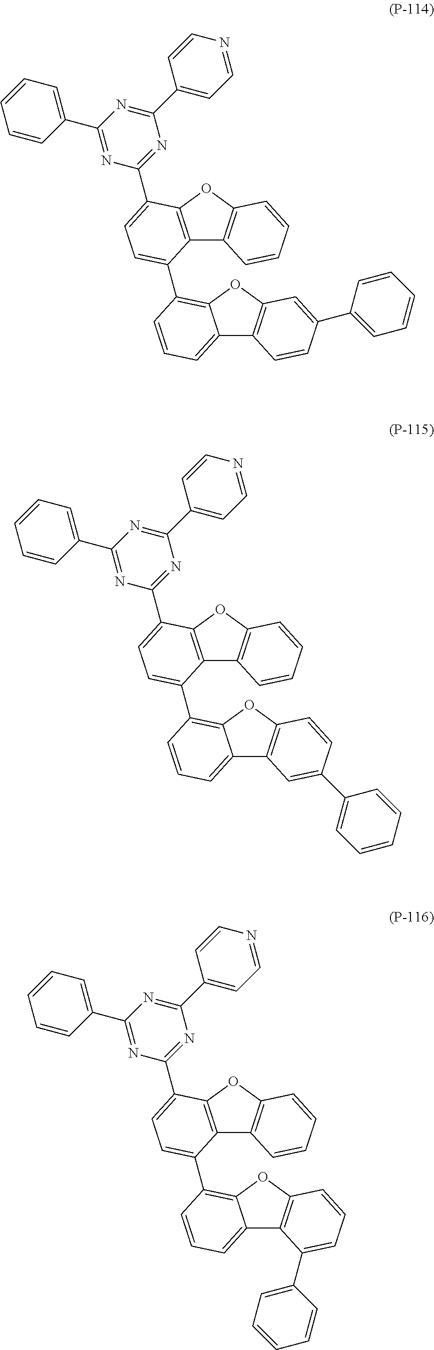 [SMILES: c1ccc(-c2ccc3c(c2)oc2c(-c4ccc(-c5nc(-c6ccccc6)nc(-c6ccncc6)n5)c5oc6ccccc6c45)cccc23)cc1 ; c1ccc(-c2ccc3oc4c(-c5ccc(-c6nc(-c7ccccc7)nc(-c7ccncc7)n6)c6oc7ccccc7c56)cccc4c3c2)cc1 ; c1ccc(-c2nc(-c3ccncc3)nc(-c3ccc(-c4cccc5c4oc4cccc(-c6ccccc6)c45)c4c3oc3ccccc34)n2)cc1]

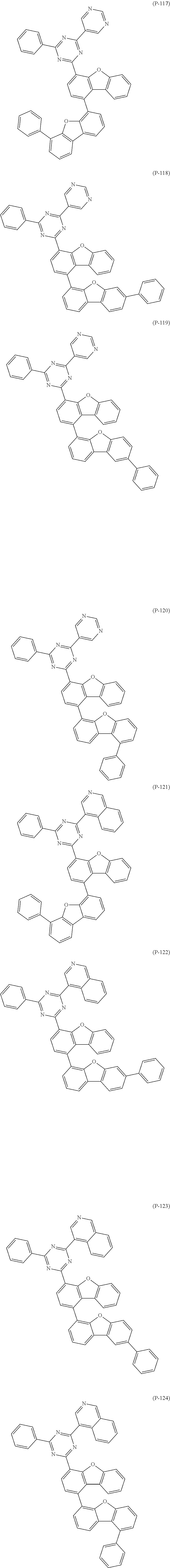

[chemical structure]

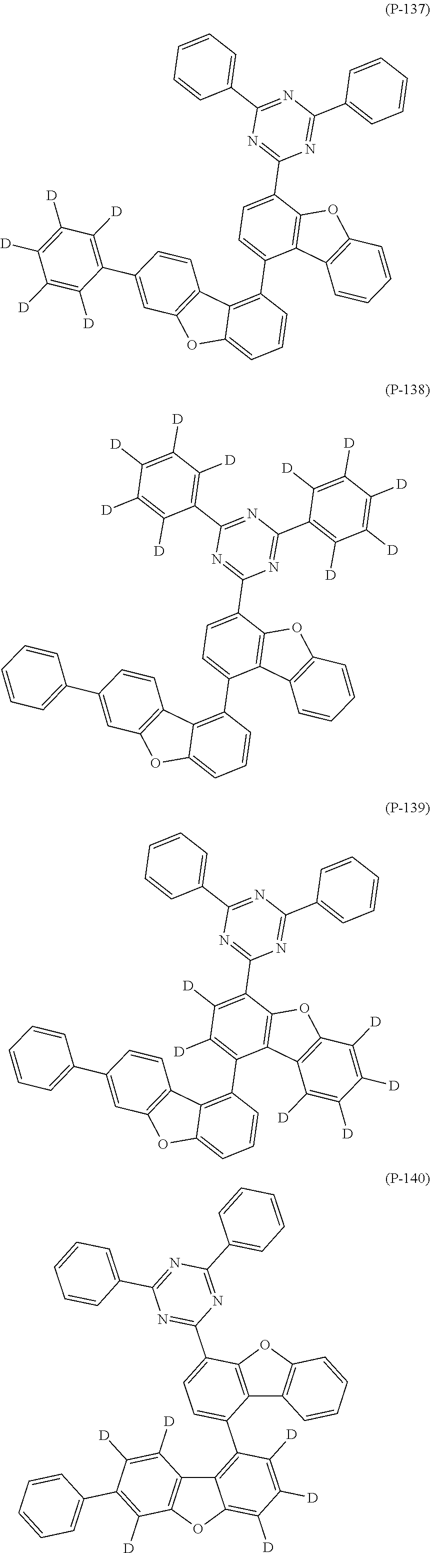

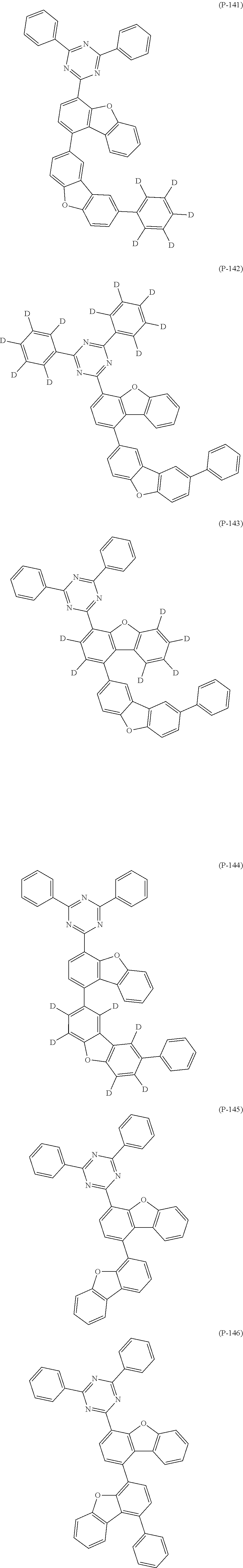

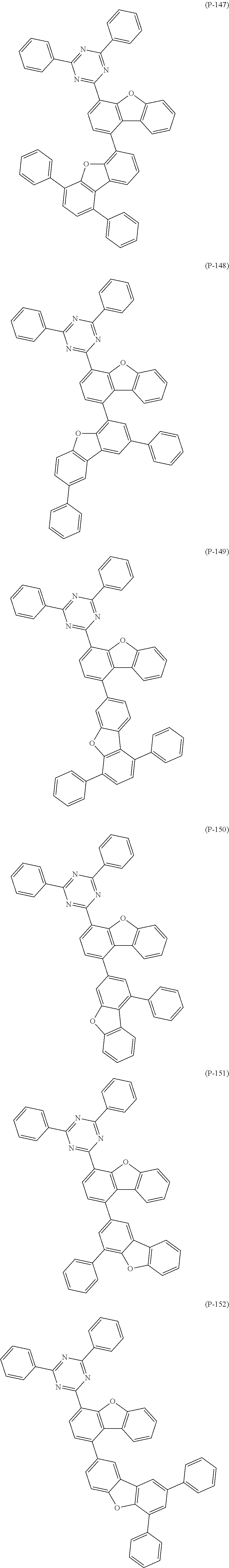

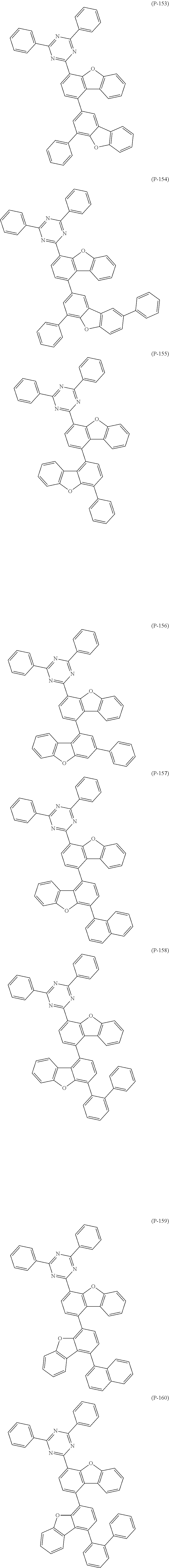

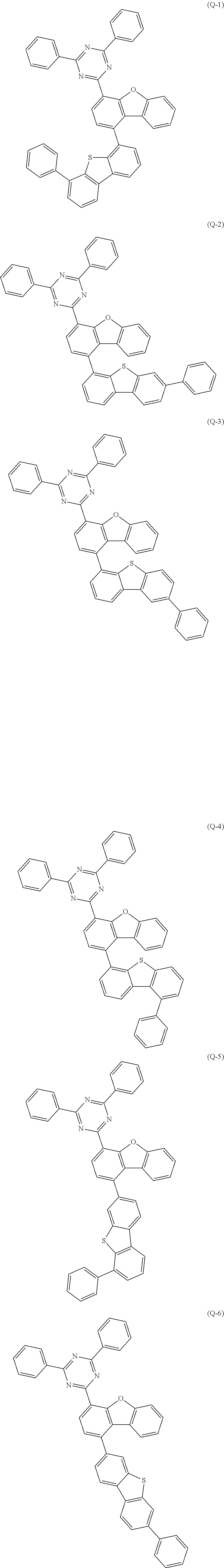

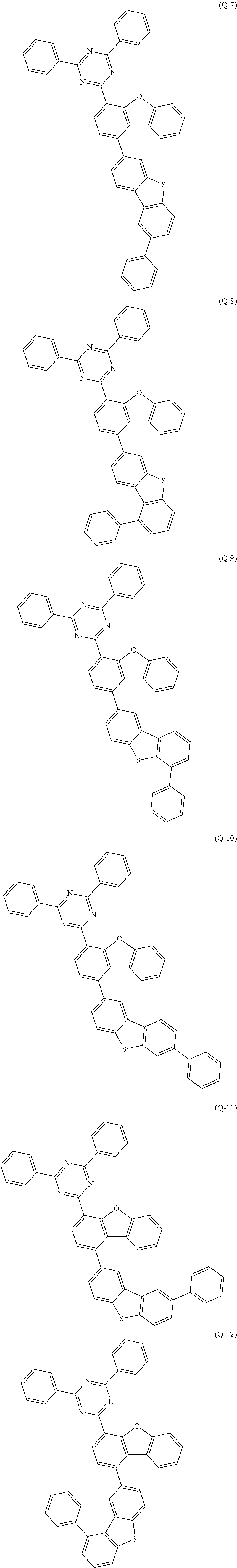

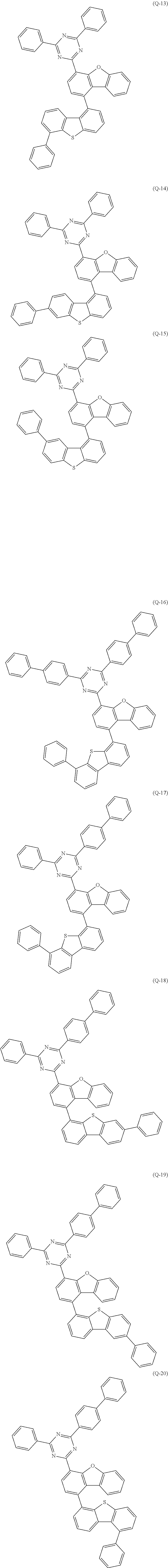

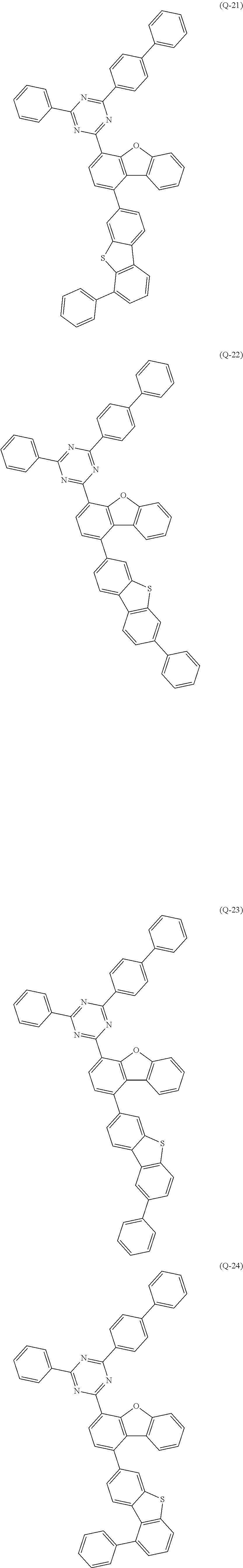

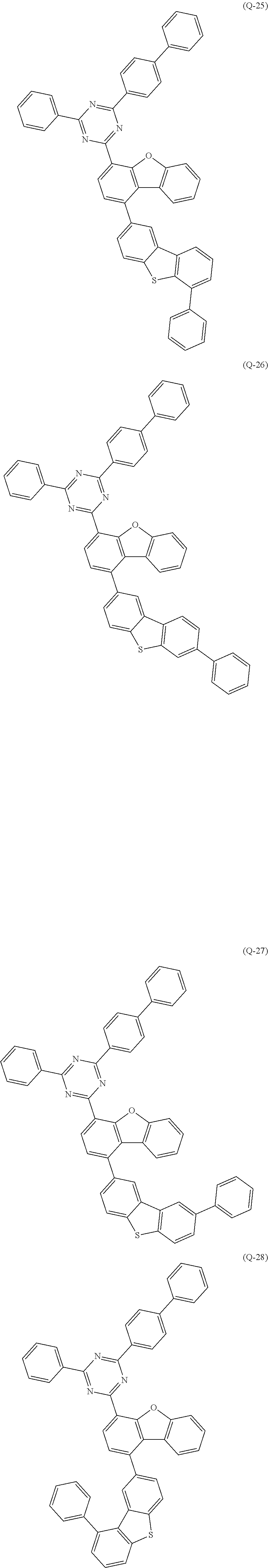

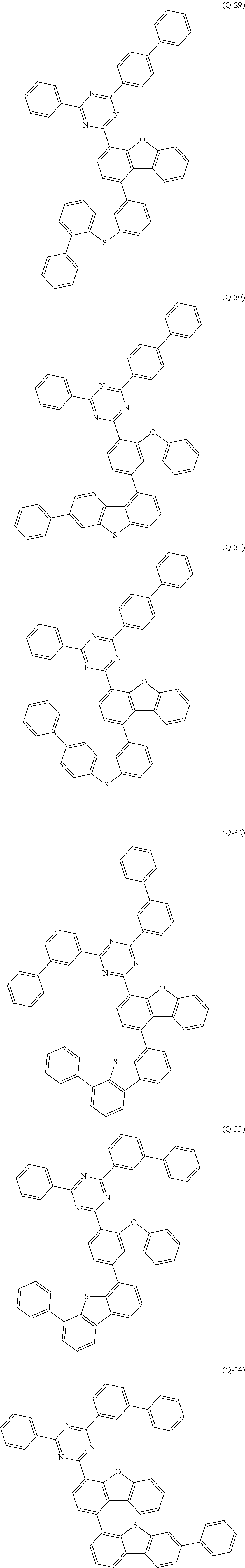

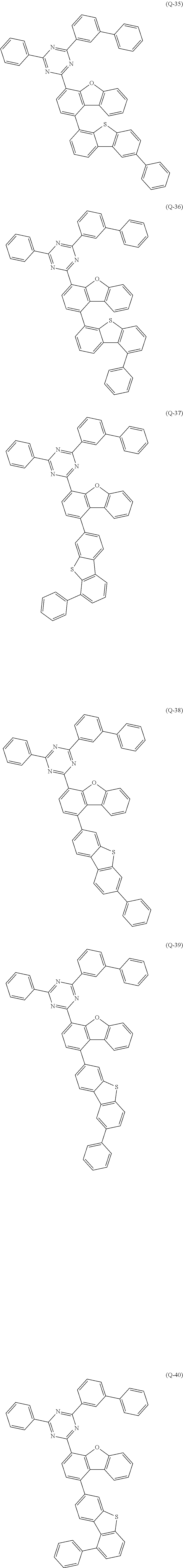

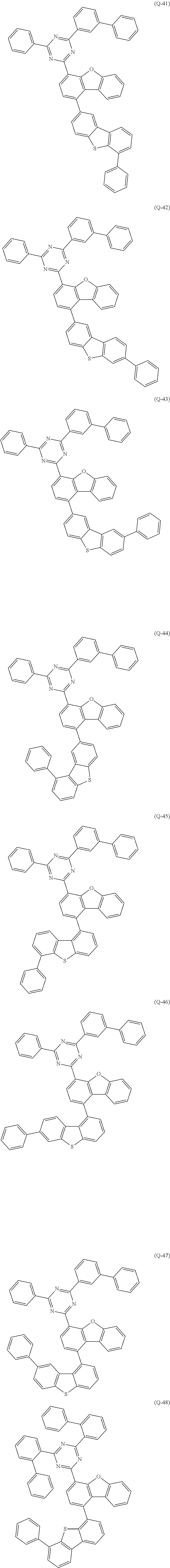

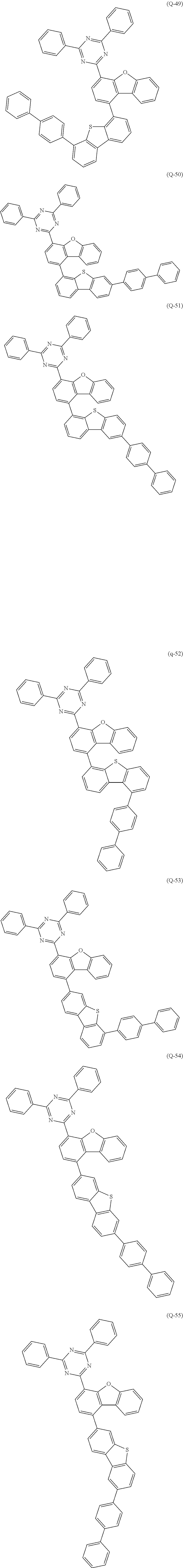

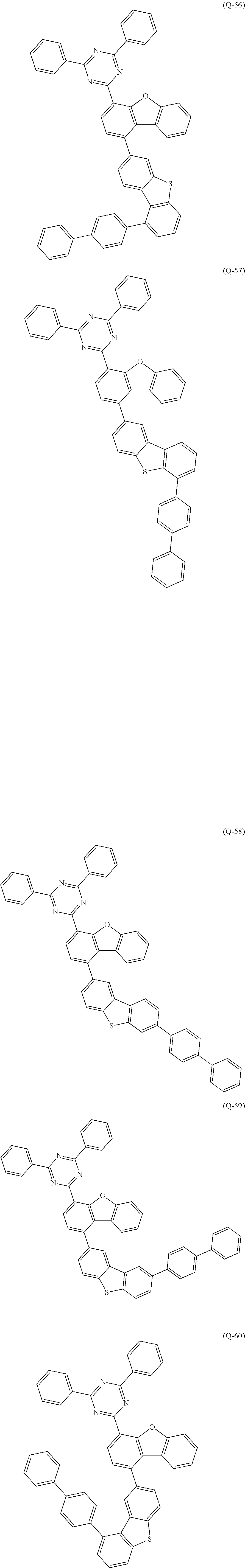 [SMILES: c1ccc(-c2ccc(-c3cccc4sc5cc(-c6ccc(-c7nc(-c8ccccc8)nc(-c8ccccc8)n7)c7oc8ccccc8c67)ccc5c34)cc2)cc1 ; c1ccc(-c2ccc(-c3cccc4c3sc3ccc(-c5ccc(-c6nc(-c7ccccc7)nc(-c7ccccc7)n6)c6oc7ccccc7c56)cc34)cc2)cc1 ; c1ccc(-c2ccc(-c3ccc4c(c3)sc3ccc(-c5ccc(-c6nc(-c7ccccc7)nc(-c7ccccc7)n6)c6oc7ccccc7c56)cc34)cc2)cc1 ; c1ccc(-c2ccc(-c3ccc4sc5ccc(-c6ccc(-c7nc(-c8ccccc8)nc(-c8ccccc8)n7)c7oc8ccccc8c67)cc5c4c3)cc2)cc1 ; c1ccc(-c2ccc(-c3cccc4sc5ccc(-c6ccc(-c7nc(-c8ccccc8)nc(-c8ccccc8)n7)c7oc8ccccc8c67)cc5c34)cc2)cc1]

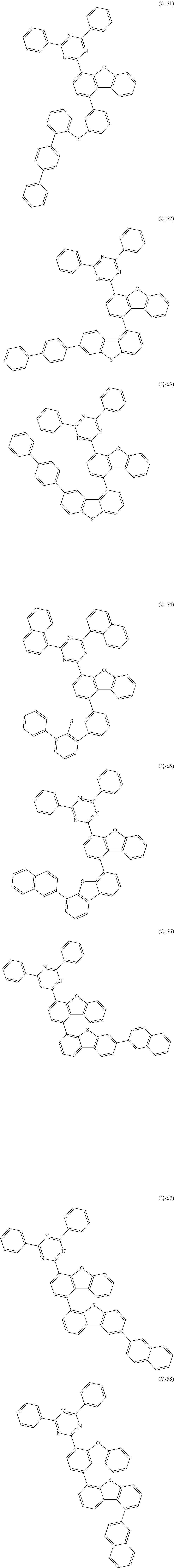

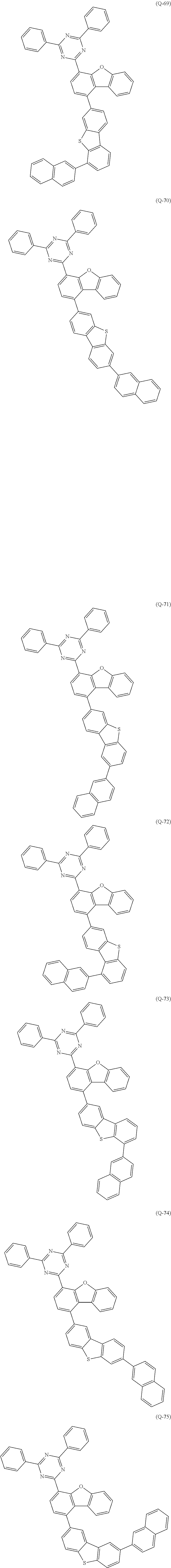

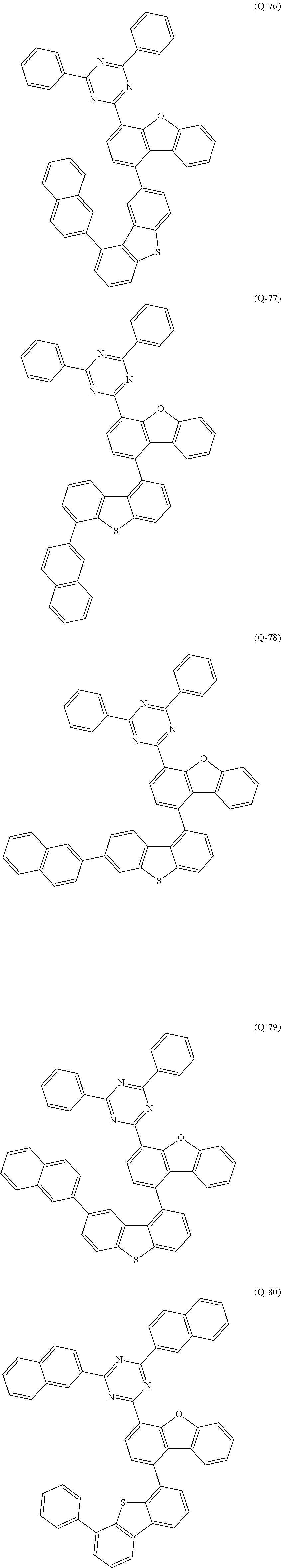

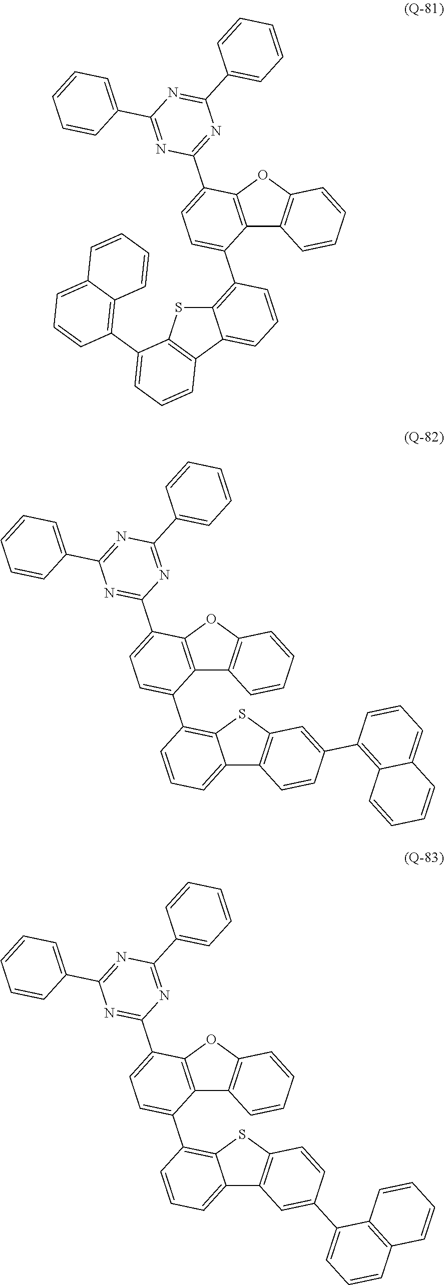 [SMILES: c1ccc(-c2nc(-c3ccccc3)nc(-c3ccc(-c4cccc5c4sc4c(-c6cccc7ccccc67)cccc45)c4c3oc3ccccc34)n2)cc1 ; c1ccc(-c2nc(-c3ccccc3)nc(-c3ccc(-c4cccc5c4sc4cc(-c6cccc7ccccc67)ccc45)c4c3oc3ccccc34)n2)cc1 ; c1ccc(-c2nc(-c3ccccc3)nc(-c3ccc(-c4cccc5c4sc4ccc(-c6cccc7ccccc67)cc45)c4c3oc3ccccc34)n2)cc1]

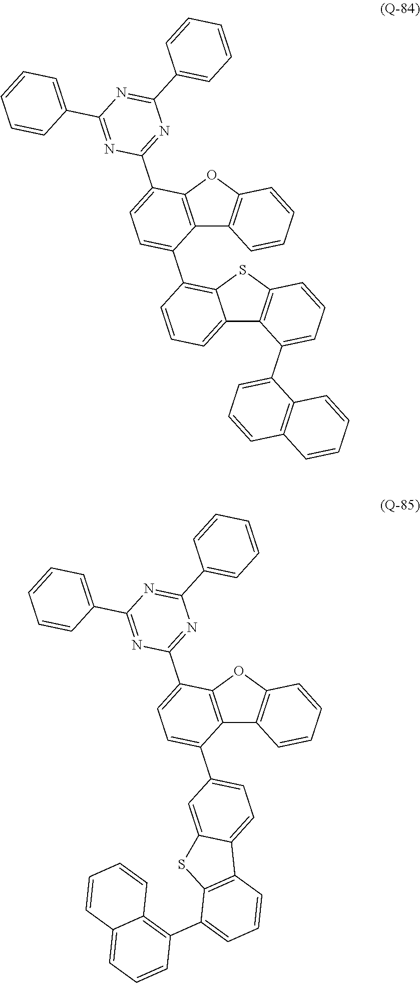 [SMILES: c1ccc(-c2nc(-c3ccccc3)nc(-c3ccc(-c4cccc5c4sc4cccc(-c6cccc7ccccc67)c45)c4c3oc3ccccc34)n2)cc1 ; c1ccc(-c2nc(-c3ccccc3)nc(-c3ccc(-c4ccc5c(c4)sc4c(-c6cccc7ccccc67)cccc45)c4c3oc3ccccc34)n2)cc1]

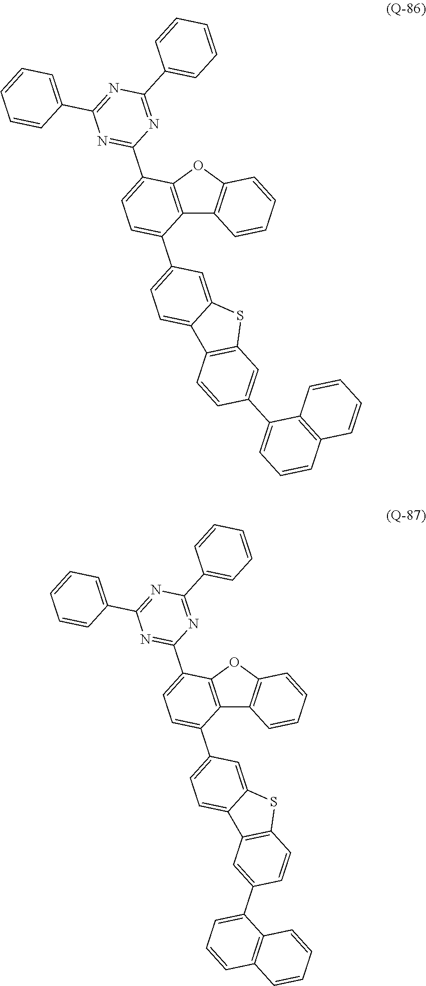 [SMILES: c1ccc(-c2nc(-c3ccccc3)nc(-c3ccc(-c4ccc5c(c4)sc4cc(-c6cccc7ccccc67)ccc45)c4c3oc3ccccc34)n2)cc1 ; c1ccc(-c2nc(-c3ccccc3)nc(-c3ccc(-c4ccc5c(c4)sc4ccc(-c6cccc7ccccc67)cc45)c4c3oc3ccccc34)n2)cc1]

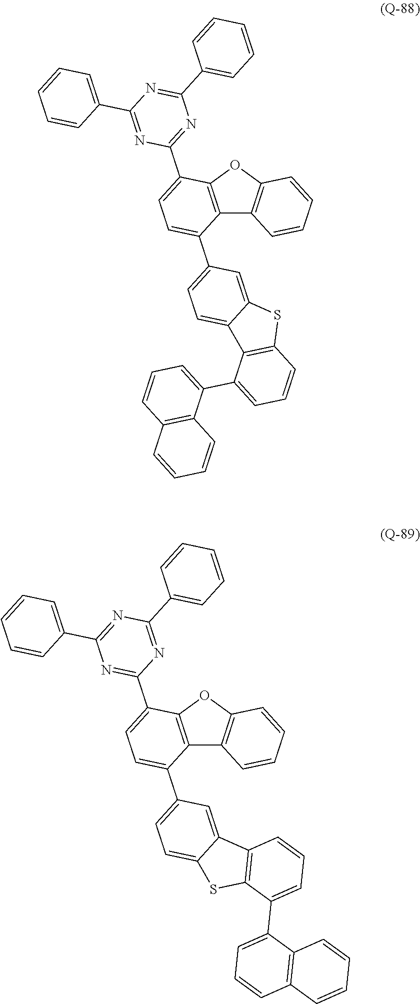 [SMILES: c1ccc(-c2nc(-c3ccccc3)nc(-c3ccc(-c4ccc5c(c4)sc4cccc(-c6cccc7ccccc67)c45)c4c3oc3ccccc34)n2)cc1 ; c1ccc(-c2nc(-c3ccccc3)nc(-c3ccc(-c4ccc5sc6c(-c7cccc8ccccc78)cccc6c5c4)c4c3oc3ccccc34)n2)cc1]

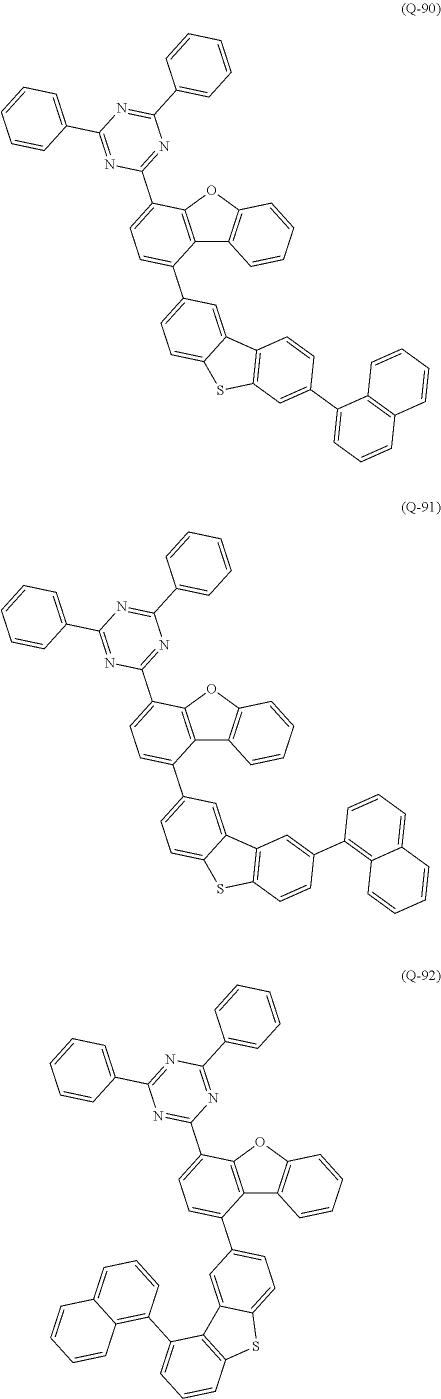 [SMILES: c1ccc(-c2nc(-c3ccccc3)nc(-c3ccc(-c4ccc5sc6cc(-c7cccc8ccccc78)ccc6c5c4)c4c3oc3ccccc34)n2)cc1 ; c1ccc(-c2nc(-c3ccccc3)nc(-c3ccc(-c4ccc5sc6ccc(-c7cccc8ccccc78)cc6c5c4)c4c3oc3ccccc34)n2)cc1 ; c1ccc(-c2nc(-c3ccccc3)nc(-c3ccc(-c4ccc5sc6cccc(-c7cccc8ccccc78)c6c5c4)c4c3oc3ccccc34)n2)cc1]

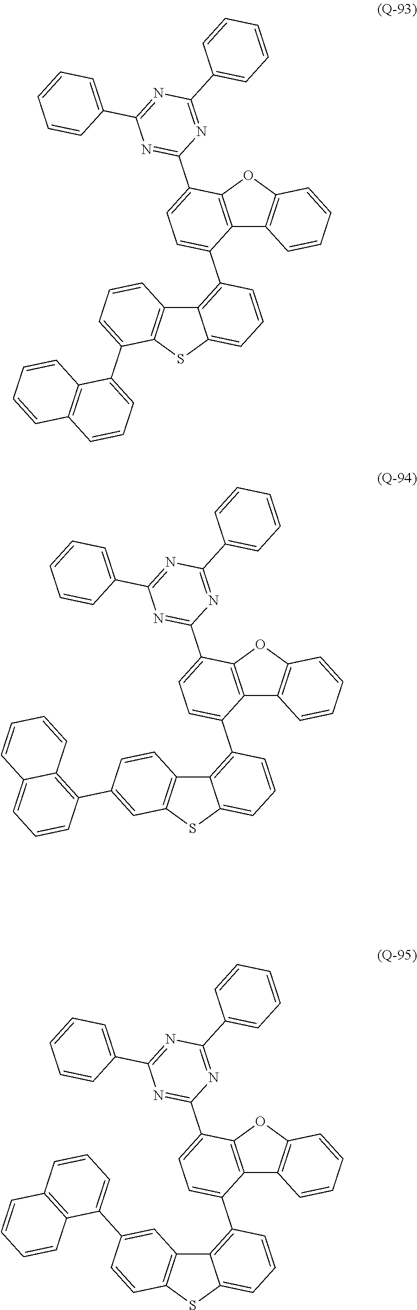 [SMILES: c1ccc(-c2nc(-c3ccccc3)nc(-c3ccc(-c4cccc5sc6c(-c7cccc8ccccc78)cccc6c45)c4c3oc3ccccc34)n2)cc1 ; c1ccc(-c2nc(-c3ccccc3)nc(-c3ccc(-c4cccc5sc6cc(-c7cccc8ccccc78)ccc6c45)c4c3oc3ccccc34)n2)cc1 ; c1ccc(-c2nc(-c3ccccc3)nc(-c3ccc(-c4cccc5sc6ccc(-c7cccc8ccccc78)cc6c45)c4c3oc3ccccc34)n2)cc1]

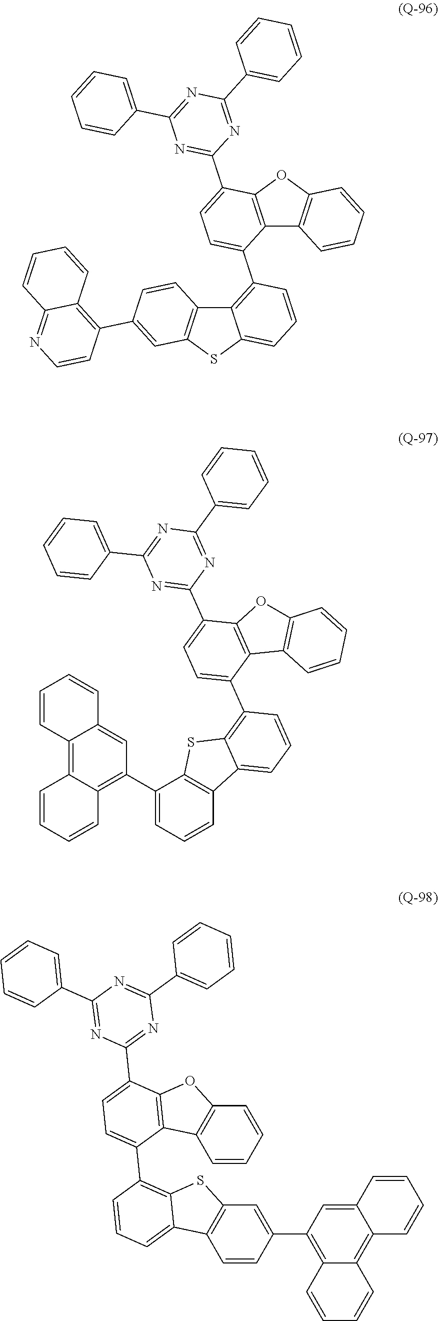 [SMILES: c1ccc(-c2nc(-c3ccccc3)nc(-c3ccc(-c4cccc5sc6cc(-c7ccnc8ccccc78)ccc6c45)c4c3oc3ccccc34)n2)cc1 ; c1ccc(-c2nc(-c3ccccc3)nc(-c3ccc(-c4cccc5c4sc4c(-c6cc7ccccc7c7ccccc67)cccc45)c4c3oc3ccccc34)n2)cc1 ; c1ccc(-c2nc(-c3ccccc3)nc(-c3ccc(-c4cccc5c4sc4cc(-c6cc7ccccc7c7ccccc67)ccc45)c4c3oc3ccccc34)n2)cc1]

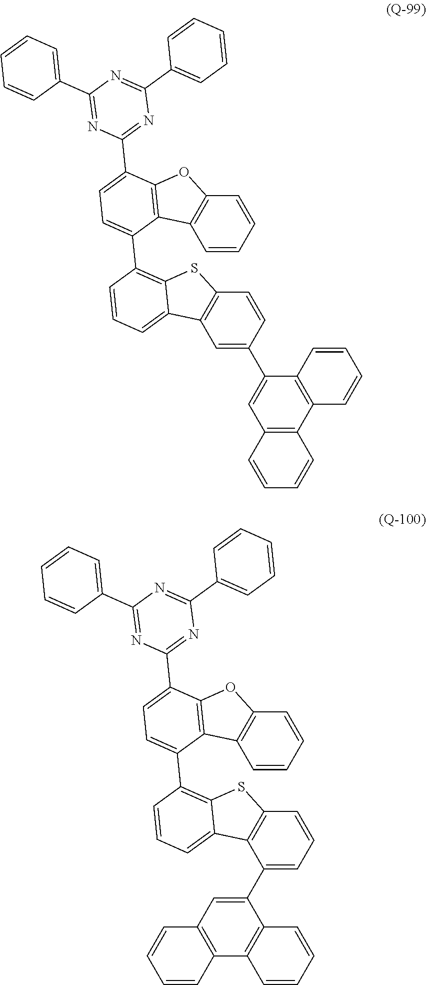 [SMILES: c1ccc(-c2nc(-c3ccccc3)nc(-c3ccc(-c4cccc5c4sc4ccc(-c6cc7ccccc7c7ccccc67)cc45)c4c3oc3ccccc34)n2)cc1 ; c1ccc(-c2nc(-c3ccccc3)nc(-c3ccc(-c4cccc5c4sc4cccc(-c6cc7ccccc7c7ccccc67)c45)c4c3oc3ccccc34)n2)cc1]

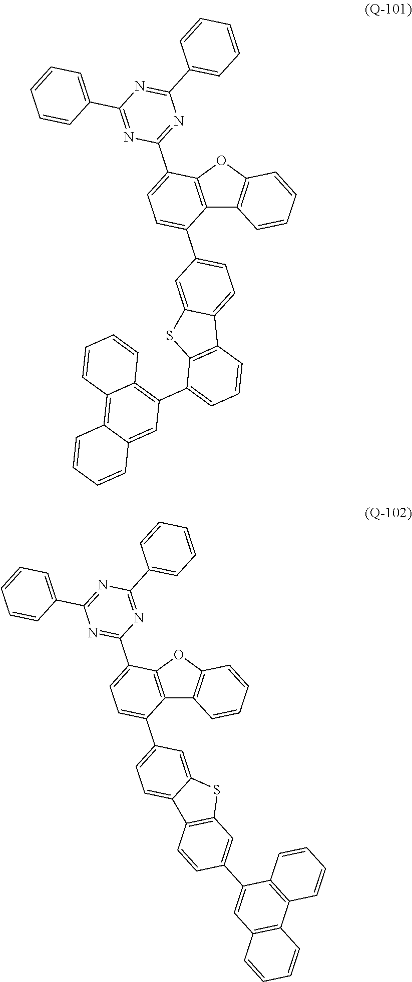 [SMILES: c1ccc(-c2nc(-c3ccccc3)nc(-c3ccc(-c4ccc5c(c4)sc4c(-c6cc7ccccc7c7ccccc67)cccc45)c4c3oc3ccccc34)n2)cc1 ; c1ccc(-c2nc(-c3ccccc3)nc(-c3ccc(-c4ccc5c(c4)sc4cc(-c6cc7ccccc7c7ccccc67)ccc45)c4c3oc3ccccc34)n2)cc1]

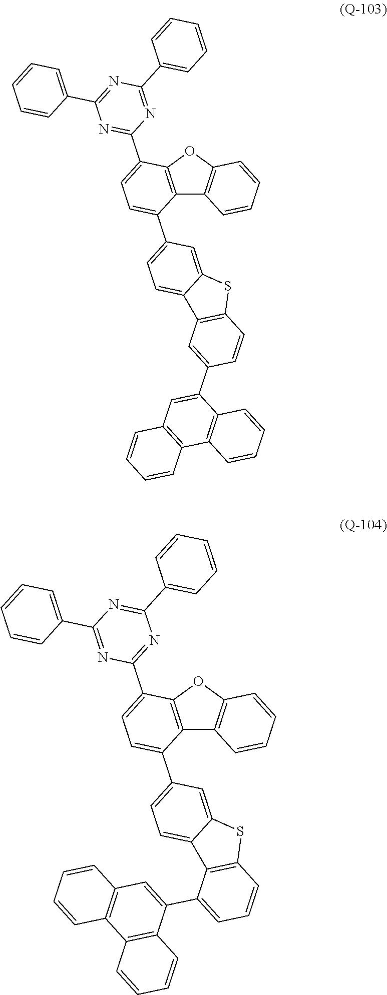 [SMILES: c1ccc(-c2nc(-c3ccccc3)nc(-c3ccc(-c4ccc5c(c4)sc4ccc(-c6cc7ccccc7c7ccccc67)cc45)c4c3oc3ccccc34)n2)cc1 ; c1ccc(-c2nc(-c3ccccc3)nc(-c3ccc(-c4ccc5c(c4)sc4cccc(-c6cc7ccccc7c7ccccc67)c45)c4c3oc3ccccc34)n2)cc1]

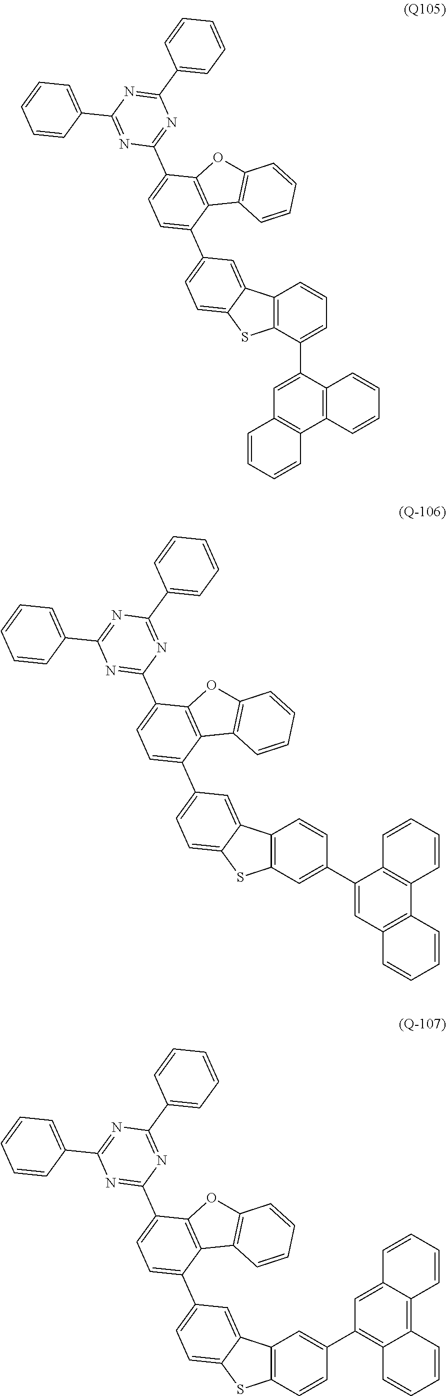 [SMILES: c1ccc(-c2nc(-c3ccccc3)nc(-c3ccc(-c4ccc5sc6c(-c7cc8ccccc8c8ccccc78)cccc6c5c4)c4c3oc3ccccc34)n2)cc1 ; c1ccc(-c2nc(-c3ccccc3)nc(-c3ccc(-c4ccc5sc6cc(-c7cc8ccccc8c8ccccc78)ccc6c5c4)c4c3oc3ccccc34)n2)cc1 ; c1ccc(-c2nc(-c3ccccc3)nc(-c3ccc(-c4ccc5sc6ccc(-c7cc8ccccc8c8ccccc78)cc6c5c4)c4c3oc3ccccc34)n2)cc1]

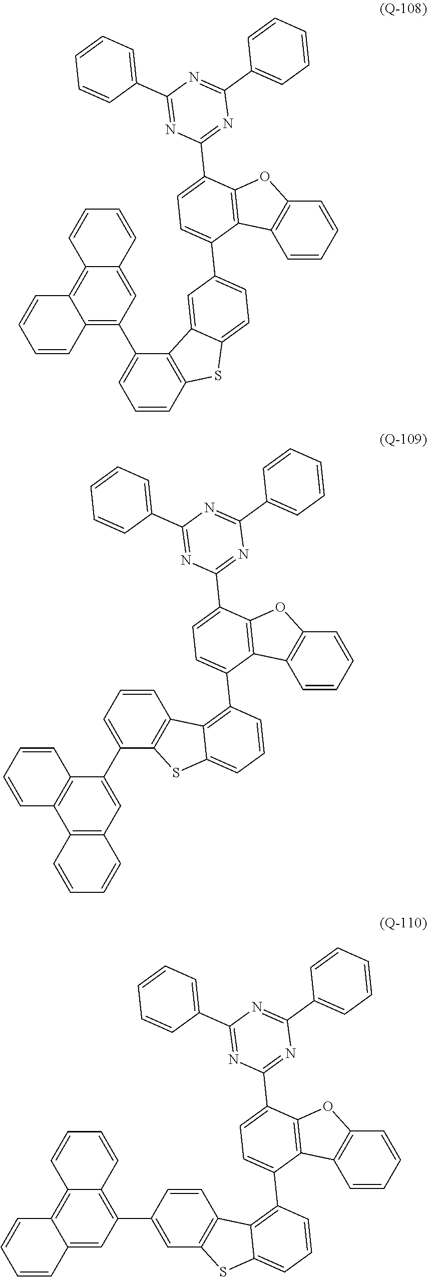 [SMILES: c1ccc(-c2nc(-c3ccccc3)nc(-c3ccc(-c4ccc5sc6cccc(-c7cc8ccccc8c8ccccc78)c6c5c4)c4c3oc3ccccc34)n2)cc1 ; c1ccc(-c2nc(-c3ccccc3)nc(-c3ccc(-c4cccc5sc6c(-c7cc8ccccc8c8ccccc78)cccc6c45)c4c3oc3ccccc34)n2)cc1 ; c1ccc(-c2nc(-c3ccccc3)nc(-c3ccc(-c4cccc5sc6cc(-c7cc8ccccc8c8ccccc78)ccc6c45)c4c3oc3ccccc34)n2)cc1]

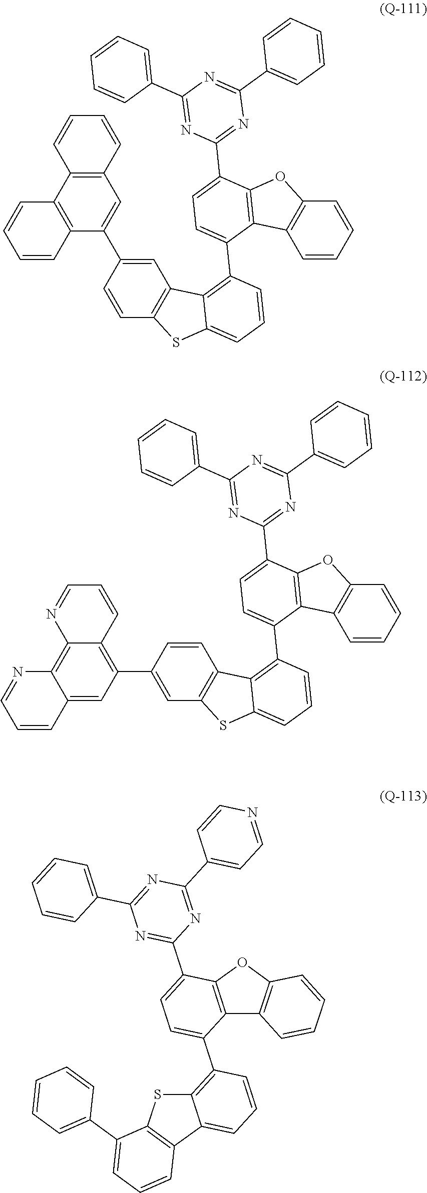 [SMILES: c1ccc(-c2nc(-c3ccccc3)nc(-c3ccc(-c4cccc5sc6ccc(-c7cc8ccccc8c8ccccc78)cc6c45)c4c3oc3ccccc34)n2)cc1 ; c1ccc(-c2nc(-c3ccccc3)nc(-c3ccc(-c4cccc5sc6cc(-c7cc8cccnc8c8ncccc78)ccc6c45)c4c3oc3ccccc34)n2)cc1 ; c1ccc(-c2nc(-c3ccncc3)nc(-c3ccc(-c4cccc5c4sc4c(-c6ccccc6)cccc45)c4c3oc3ccccc34)n2)cc1]

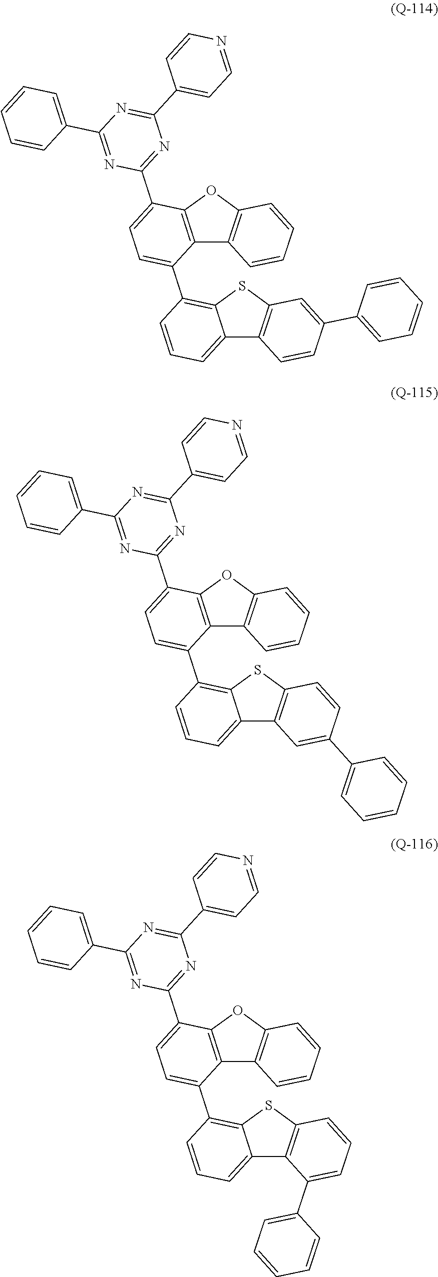 [SMILES: c1ccc(-c2ccc3c(c2)sc2c(-c4ccc(-c5nc(-c6ccccc6)nc(-c6ccncc6)n5)c5oc6ccccc6c45)cccc23)cc1 ; c1ccc(-c2ccc3sc4c(-c5ccc(-c6nc(-c7ccccc7)nc(-c7ccncc7)n6)c6oc7ccccc7c56)cccc4c3c2)cc1 ; c1ccc(-c2nc(-c3ccncc3)nc(-c3ccc(-c4cccc5c4sc4cccc(-c6ccccc6)c45)c4c3oc3ccccc34)n2)cc1]

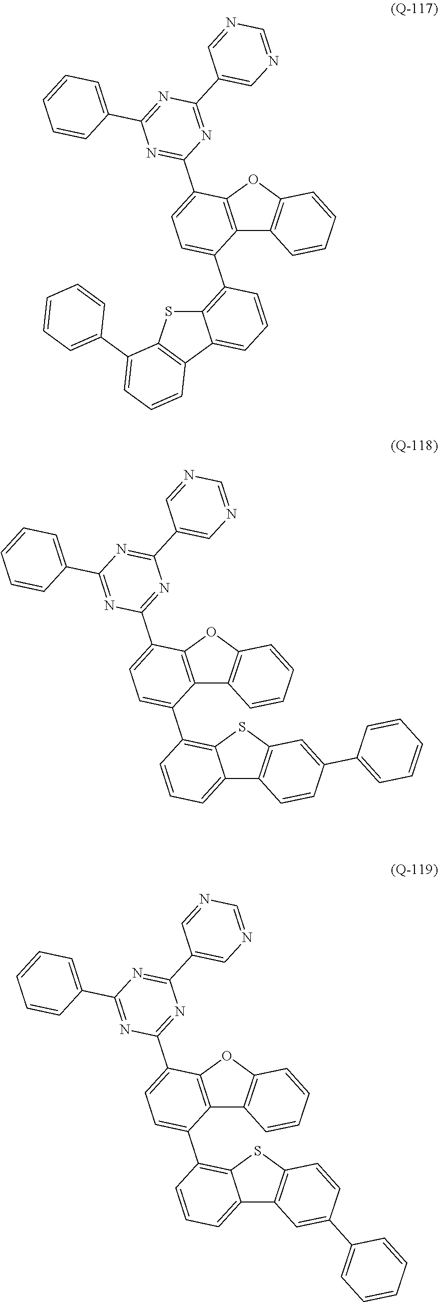 [SMILES: c1ccc(-c2nc(-c3cncnc3)nc(-c3ccc(-c4cccc5c4sc4c(-c6ccccc6)cccc45)c4c3oc3ccccc34)n2)cc1 ; c1ccc(-c2ccc3c(c2)sc2c(-c4ccc(-c5nc(-c6ccccc6)nc(-c6cncnc6)n5)c5oc6ccccc6c45)cccc23)cc1 ; c1ccc(-c2ccc3sc4c(-c5ccc(-c6nc(-c7ccccc7)nc(-c7cncnc7)n6)c6oc7ccccc7c56)cccc4c3c2)cc1]

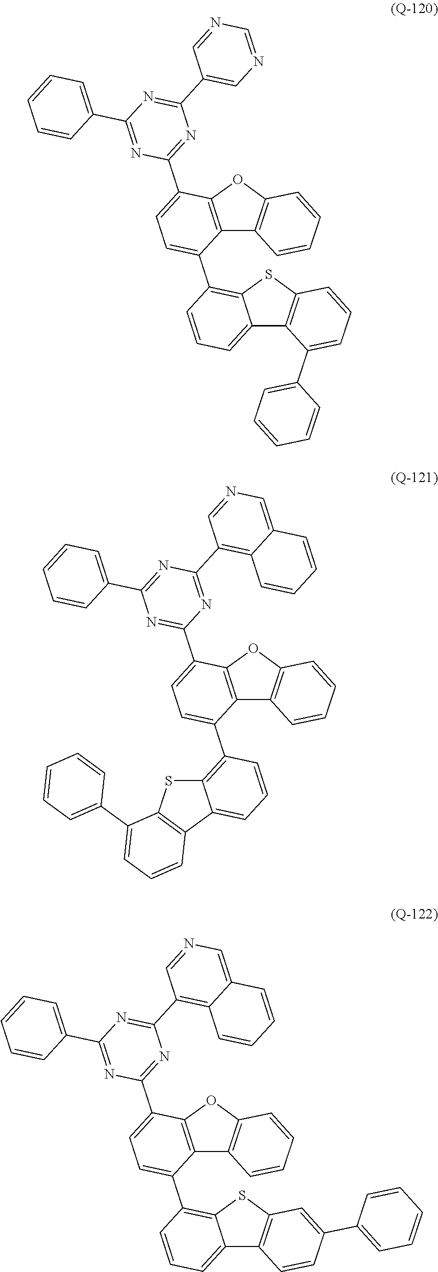 [SMILES: c1ccc(-c2nc(-c3cncnc3)nc(-c3ccc(-c4cccc5c4sc4cccc(-c6ccccc6)c45)c4c3oc3ccccc34)n2)cc1 ; c1ccc(-c2nc(-c3cncc4ccccc34)nc(-c3ccc(-c4cccc5c4sc4c(-c6ccccc6)cccc45)c4c3oc3ccccc34)n2)cc1 ; c1ccc(-c2ccc3c(c2)sc2c(-c4ccc(-c5nc(-c6ccccc6)nc(-c6cncc7ccccc67)n5)c5oc6ccccc6c45)cccc23)cc1]

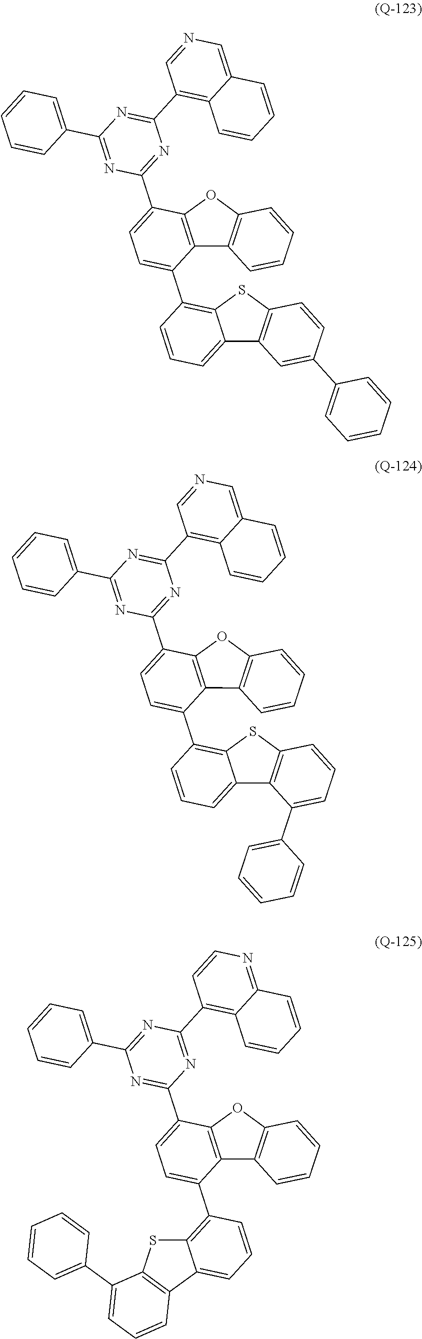 [SMILES: c1ccc(-c2ccc3sc4c(-c5ccc(-c6nc(-c7ccccc7)nc(-c7cncc8ccccc78)n6)c6oc7ccccc7c56)cccc4c3c2)cc1 ; c1ccc(-c2nc(-c3cncc4ccccc34)nc(-c3ccc(-c4cccc5c4sc4cccc(-c6ccccc6)c45)c4c3oc3ccccc34)n2)cc1 ; c1ccc(-c2nc(-c3ccnc4ccccc34)nc(-c3ccc(-c4cccc5c4sc4c(-c6ccccc6)cccc45)c4c3oc3ccccc34)n2)cc1]

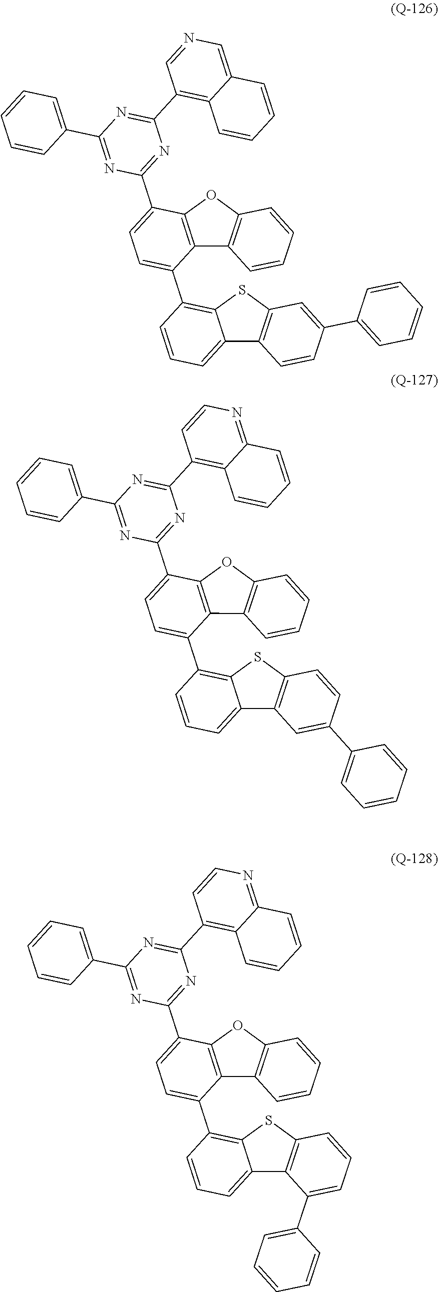

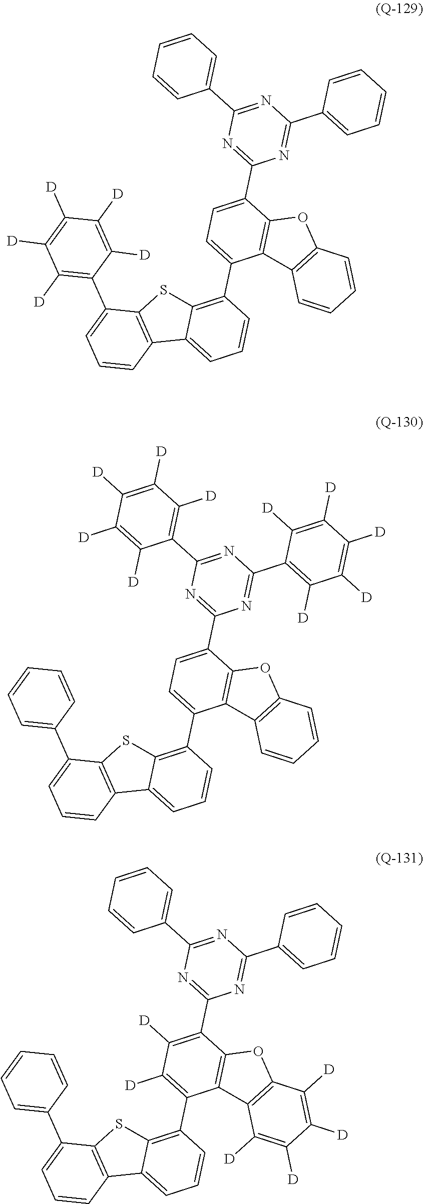 [SMILES: [2H]c1c([2H])c([2H])c(-c2cccc3c2sc2c(-c4ccc(-c5nc(-c6ccccc6)nc(-c6ccccc6)n5)c5oc6ccccc6c45)cccc23)c([2H])c1[2H] ; [2H]c1c([2H])c([2H])c(-c2nc(-c3c([2H])c([2H])c([2H])c([2H])c3[2H])nc(-c3ccc(-c4cccc5c4sc4c(-c6ccccc6)cccc45)c4c3oc3ccccc34)n2)c([2H])c1[2H] ; [2H]c1c([2H])c([2H])c2c(oc3c(-c4nc(-c5ccccc5)nc(-c5ccccc5)n4)c([2H])c([2H])c(-c4cccc5c4sc4c(-c6ccccc6)cccc45)c32)c1[2H]]

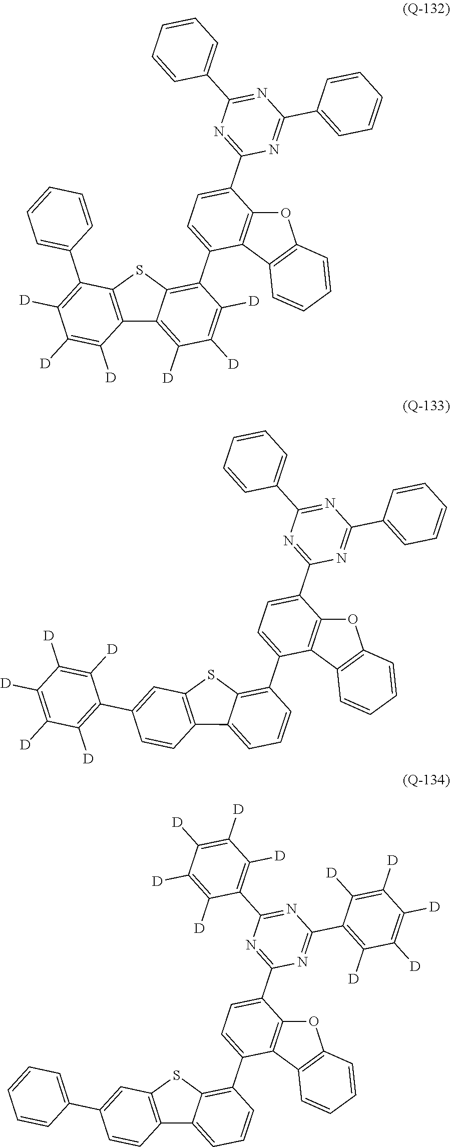 [SMILES: [2H]c1c([2H])c([2H])c2c(sc3c(-c4ccc(-c5nc(-c6ccccc6)nc(-c6ccccc6)n5)c5oc6ccccc6c45)c([2H])c([2H])c([2H])c32)c1-c1ccccc1 ; [2H]c1c([2H])c([2H])c(-c2ccc3c(c2)sc2c(-c4ccc(-c5nc(-c6ccccc6)nc(-c6ccccc6)n5)c5oc6ccccc6c45)cccc23)c([2H])c1[2H] ; [2H]c1c([2H])c([2H])c(-c2nc(-c3c([2H])c([2H])c([2H])c([2H])c3[2H])nc(-c3ccc(-c4cccc5c4sc4cc(-c6ccccc6)ccc45)c4c3oc3ccccc34)n2)c([2H])c1[2H]]

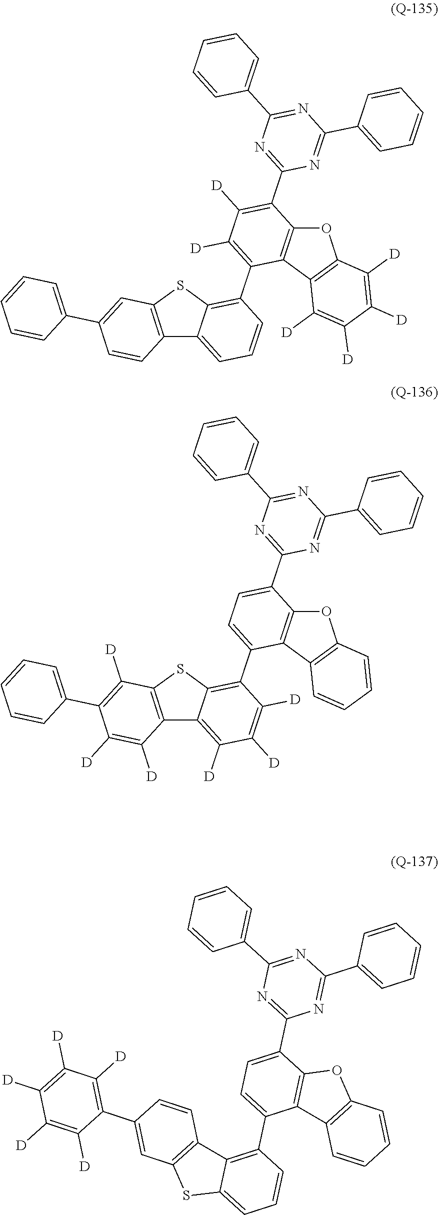 [SMILES: [2H]c1c([2H])c([2H])c2c(oc3c(-c4nc(-c5ccccc5)nc(-c5ccccc5)n4)c([2H])c([2H])c(-c4cccc5c4sc4cc(-c6ccccc6)ccc45)c32)c1[2H] ; [2H]c1c([2H])c([2H])c2c(sc3c([2H])c(-c4ccccc4)c([2H])c([2H])c32)c1-c1ccc(-c2nc(-c3ccccc3)nc(-c3ccccc3)n2)c2oc3ccccc3c12 ; [2H]c1c([2H])c([2H])c(-c2ccc3c(c2)sc2cccc(-c4ccc(-c5nc(-c6ccccc6)nc(-c6ccccc6)n5)c5oc6ccccc6c45)c23)c([2H])c1[2H]]

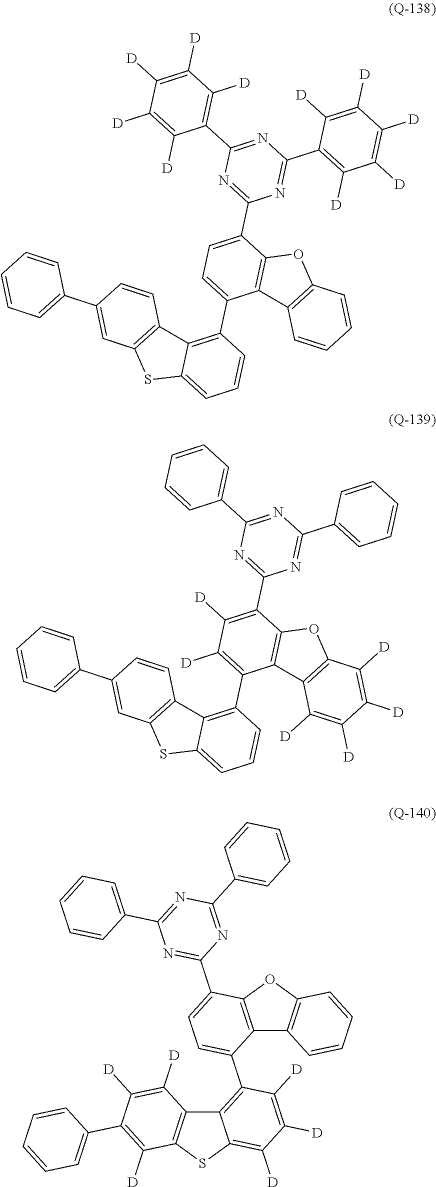

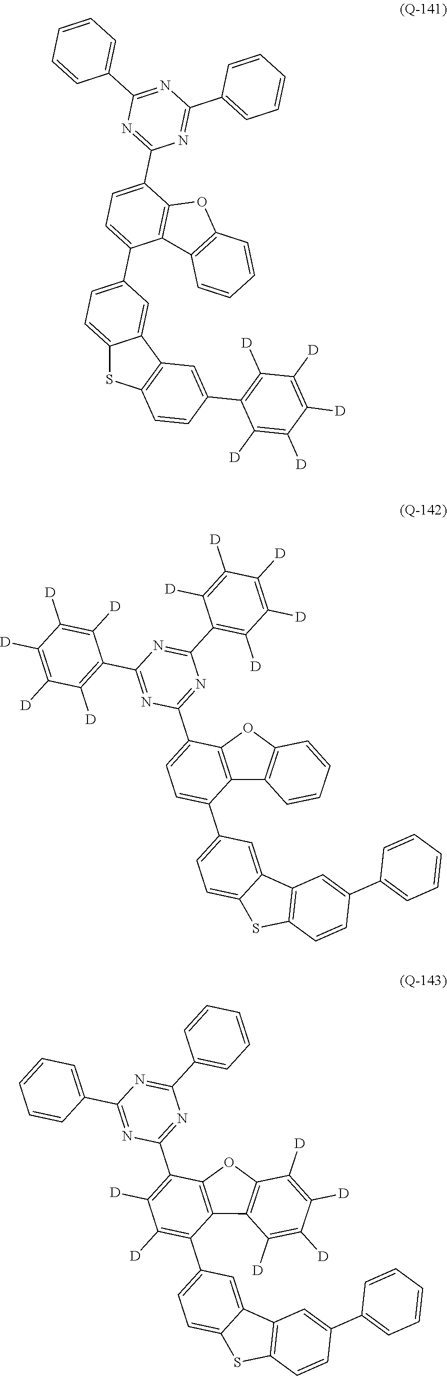 [SMILES: [2H]c1c([2H])c([2H])c(-c2ccc3sc4ccc(-c5ccc(-c6nc(-c7ccccc7)nc(-c7ccccc7)n6)c6oc7ccccc7c56)cc4c3c2)c([2H])c1[2H] ; [2H]c1c([2H])c([2H])c(-c2nc(-c3c([2H])c([2H])c([2H])c([2H])c3[2H])nc(-c3ccc(-c4ccc5sc6ccc(-c7ccccc7)cc6c5c4)c4c3oc3ccccc34)n2)c([2H])c1[2H] ; [2H]c1c([2H])c([2H])c2c(oc3c(-c4nc(-c5ccccc5)nc(-c5ccccc5)n4)c([2H])c([2H])c(-c4ccc5sc6ccc(-c7ccccc7)cc6c5c4)c32)c1[2H]]

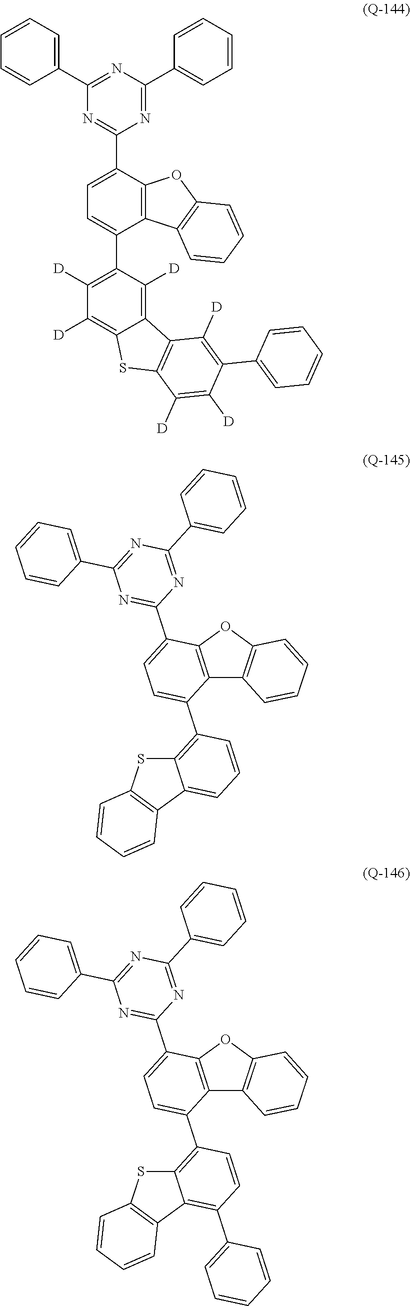 [SMILES: [2H]c1c(-c2ccccc2)c([2H])c2c(sc3c([2H])c([2H])c(-c4ccc(-c5nc(-c6ccccc6)nc(-c6ccccc6)n5)c5oc6ccccc6c45)c([2H])c32)c1[2H] ; c1ccc(-c2nc(-c3ccccc3)nc(-c3ccc(-c4cccc5c4sc4ccccc45)c4c3oc3ccccc34)n2)cc1 ; c1ccc(-c2nc(-c3ccccc3)nc(-c3ccc(-c4ccc(-c5ccccc5)c5c4sc4ccccc45)c4c3oc3ccccc34)n2)cc1]

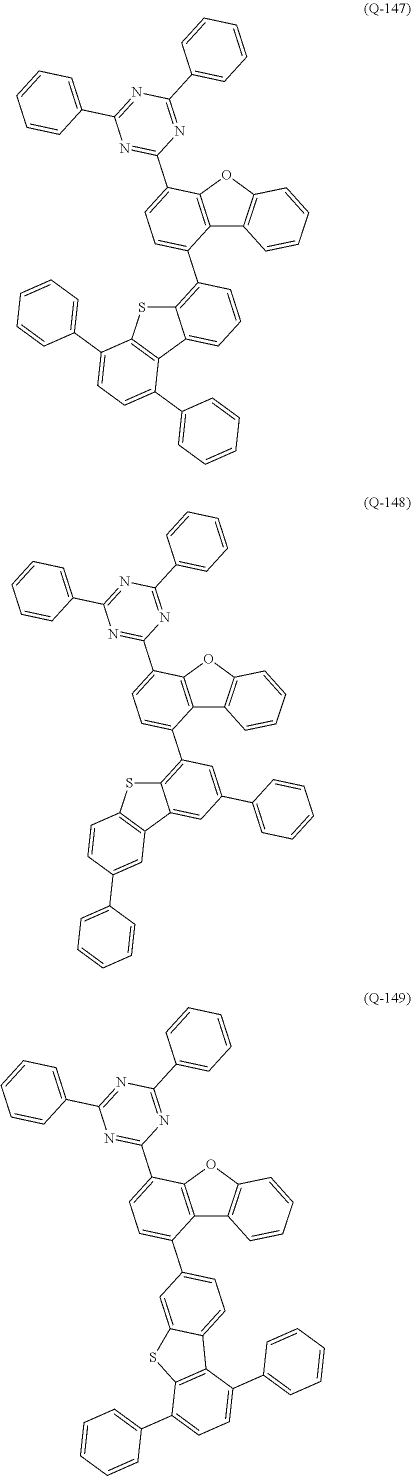 [SMILES: c1ccc(-c2nc(-c3ccccc3)nc(-c3ccc(-c4cccc5c4sc4c(-c6ccccc6)ccc(-c6ccccc6)c45)c4c3oc3ccccc34)n2)cc1 ; c1ccc(-c2ccc3sc4c(-c5ccc(-c6nc(-c7ccccc7)nc(-c7ccccc7)n6)c6oc7ccccc7c56)cc(-c5ccccc5)cc4c3c2)cc1 ; c1ccc(-c2nc(-c3ccccc3)nc(-c3ccc(-c4ccc5c(c4)sc4c(-c6ccccc6)ccc(-c6ccccc6)c45)c4c3oc3ccccc34)n2)cc1]

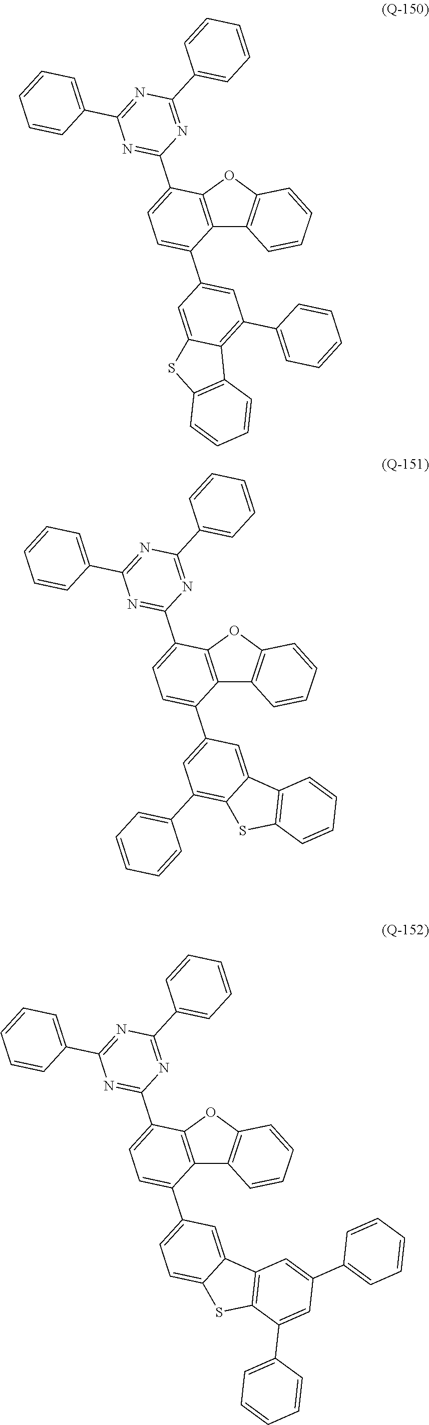

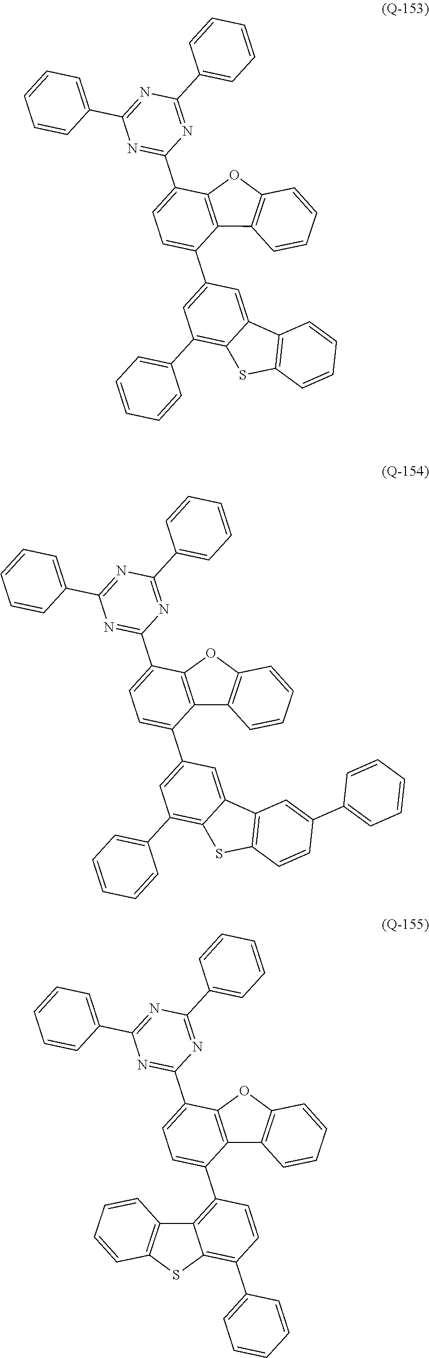 [SMILES: c1ccc(-c2nc(-c3ccccc3)nc(-c3ccc(-c4cc(-c5ccccc5)c5sc6ccccc6c5c4)c4c3oc3ccccc34)n2)cc1 ; c1ccc(-c2ccc3sc4c(-c5ccccc5)cc(-c5ccc(-c6nc(-c7ccccc7)nc(-c7ccccc7)n6)c6oc7ccccc7c56)cc4c3c2)cc1 ; c1ccc(-c2nc(-c3ccccc3)nc(-c3ccc(-c4ccc(-c5ccccc5)c5sc6ccccc6c45)c4c3oc3ccccc34)n2)cc1]

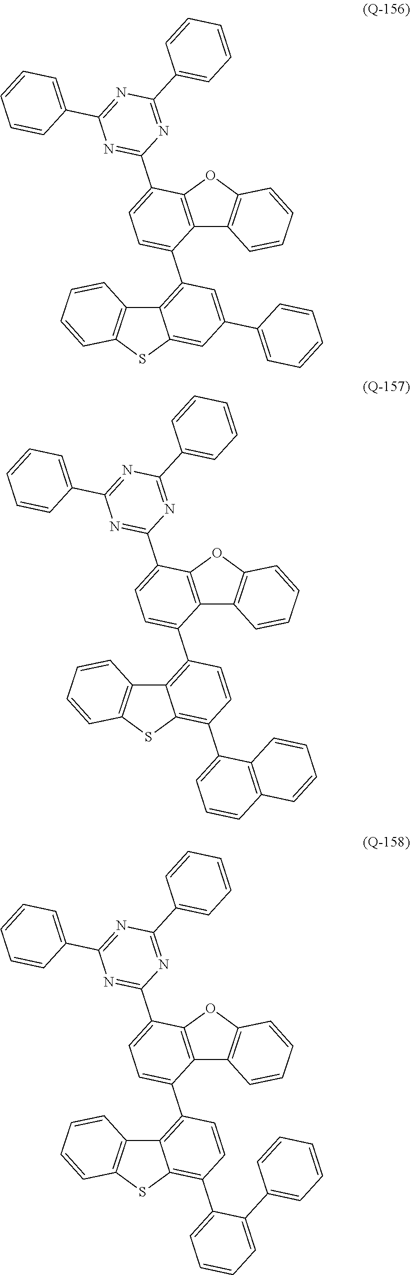

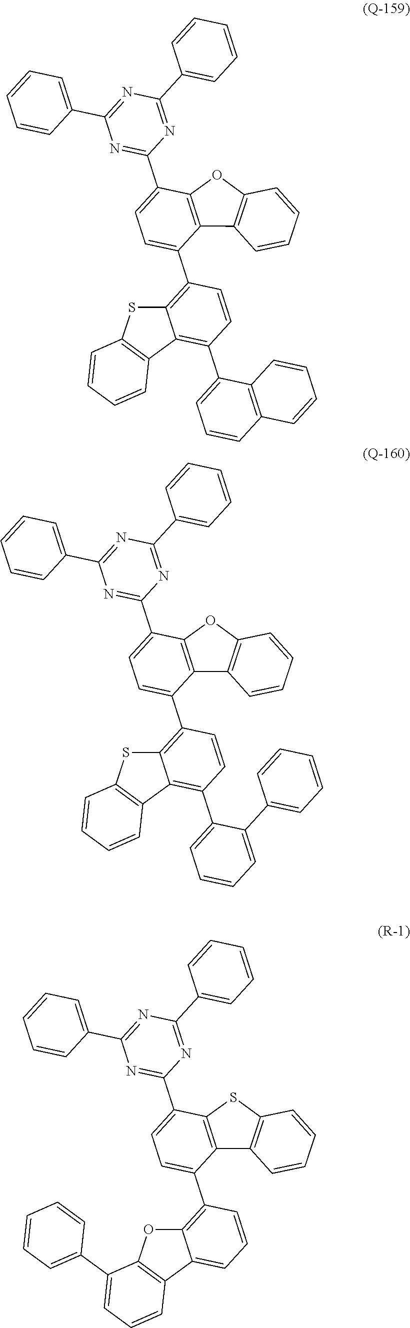

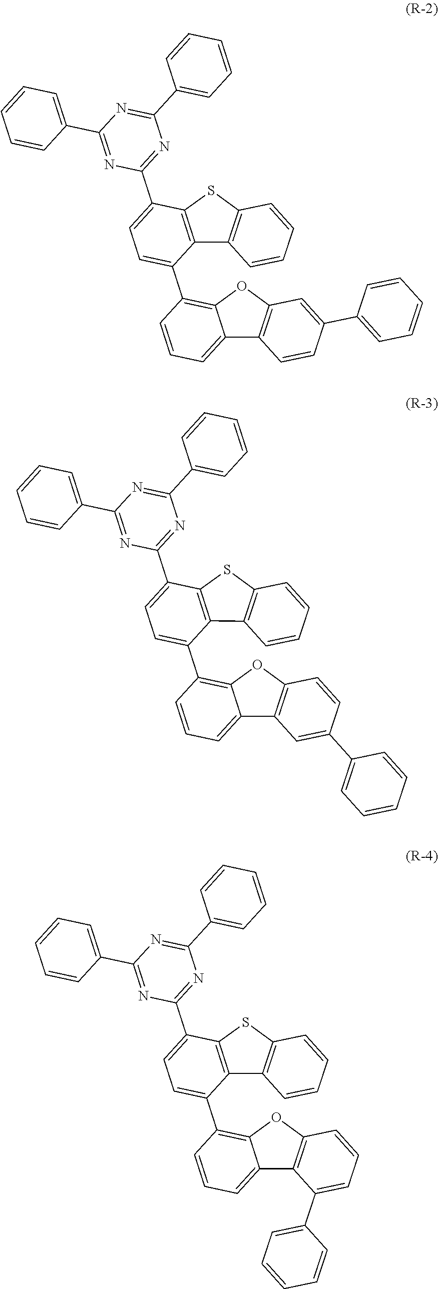 [SMILES: c1ccc(-c2ccc3c(c2)oc2c(-c4ccc(-c5nc(-c6ccccc6)nc(-c6ccccc6)n5)c5sc6ccccc6c45)cccc23)cc1 ; c1ccc(-c2ccc3oc4c(-c5ccc(-c6nc(-c7ccccc7)nc(-c7ccccc7)n6)c6sc7ccccc7c56)cccc4c3c2)cc1 ; c1ccc(-c2nc(-c3ccccc3)nc(-c3ccc(-c4cccc5c4oc4cccc(-c6ccccc6)c45)c4c3sc3ccccc34)n2)cc1]

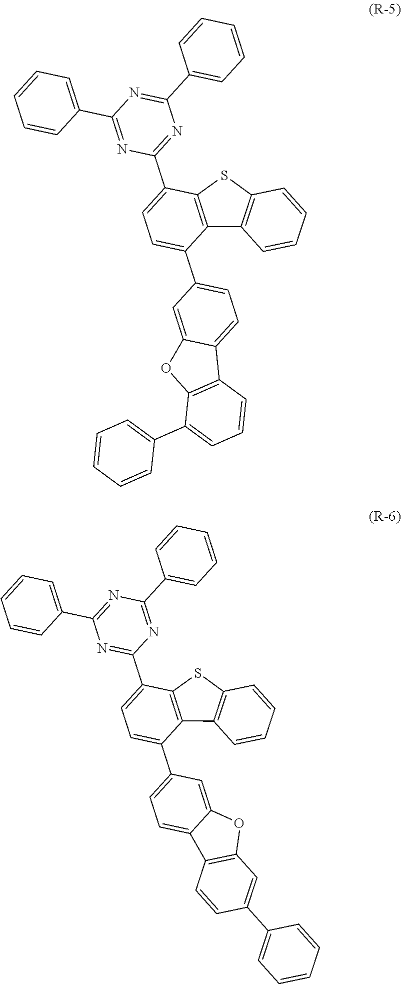 [SMILES: c1ccc(-c2nc(-c3ccccc3)nc(-c3ccc(-c4ccc5c(c4)oc4c(-c6ccccc6)cccc45)c4c3sc3ccccc34)n2)cc1 ; c1ccc(-c2ccc3c(c2)oc2cc(-c4ccc(-c5nc(-c6ccccc6)nc(-c6ccccc6)n5)c5sc6ccccc6c45)ccc23)cc1]

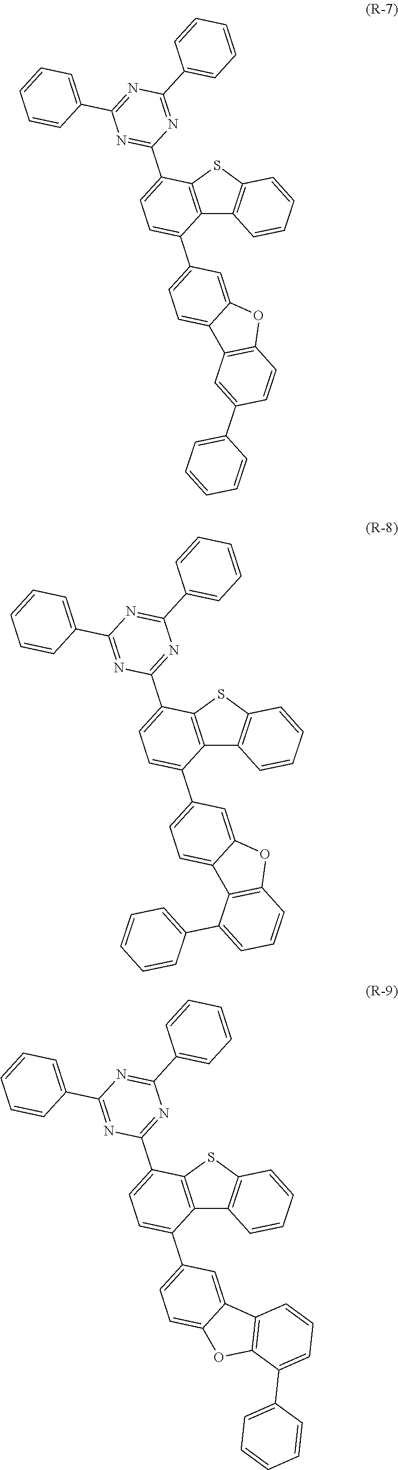

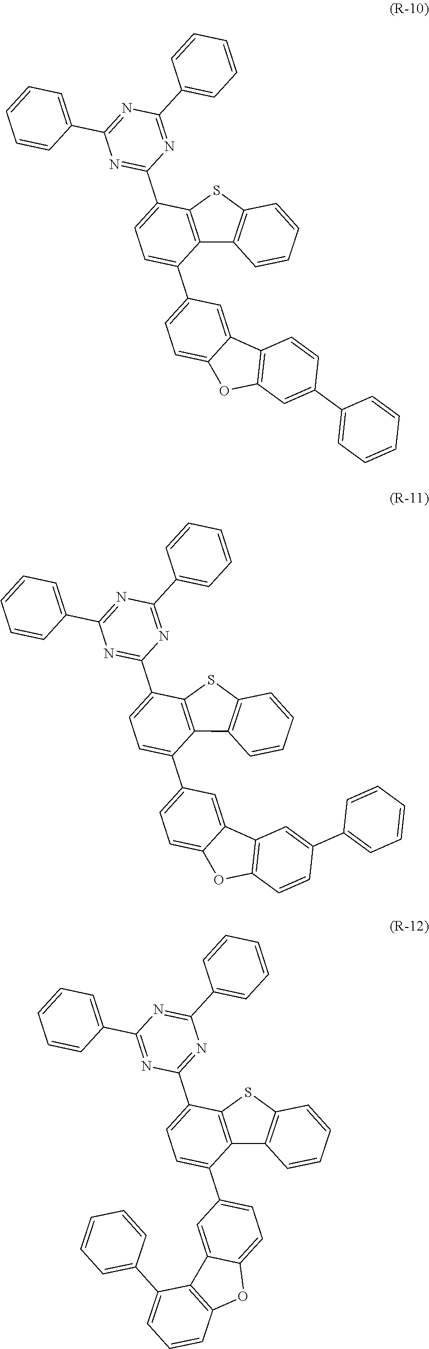 [SMILES: c1ccc(-c2ccc3c(c2)oc2ccc(-c4ccc(-c5nc(-c6ccccc6)nc(-c6ccccc6)n5)c5sc6ccccc6c45)cc23)cc1 ; c1ccc(-c2ccc3oc4ccc(-c5ccc(-c6nc(-c7ccccc7)nc(-c7ccccc7)n6)c6sc7ccccc7c56)cc4c3c2)cc1 ; c1ccc(-c2nc(-c3ccccc3)nc(-c3ccc(-c4ccc5oc6cccc(-c7ccccc7)c6c5c4)c4c3sc3ccccc34)n2)cc1]

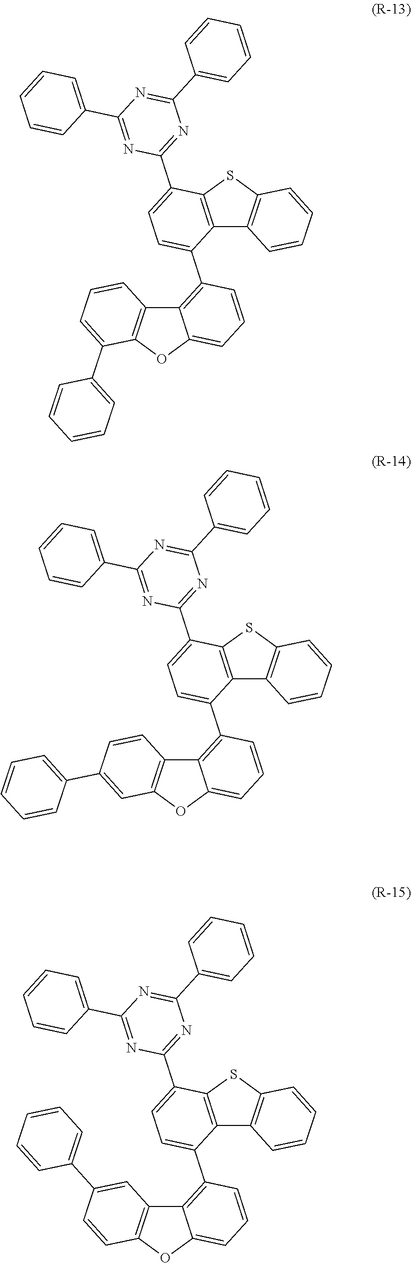 [SMILES: c1ccc(-c2nc(-c3ccccc3)nc(-c3ccc(-c4cccc5oc6c(-c7ccccc7)cccc6c45)c4c3sc3ccccc34)n2)cc1 ; c1ccc(-c2ccc3c(c2)oc2cccc(-c4ccc(-c5nc(-c6ccccc6)nc(-c6ccccc6)n5)c5sc6ccccc6c45)c23)cc1 ; c1ccc(-c2ccc3oc4cccc(-c5ccc(-c6nc(-c7ccccc7)nc(-c7ccccc7)n6)c6sc7ccccc7c56)c4c3c2)cc1]

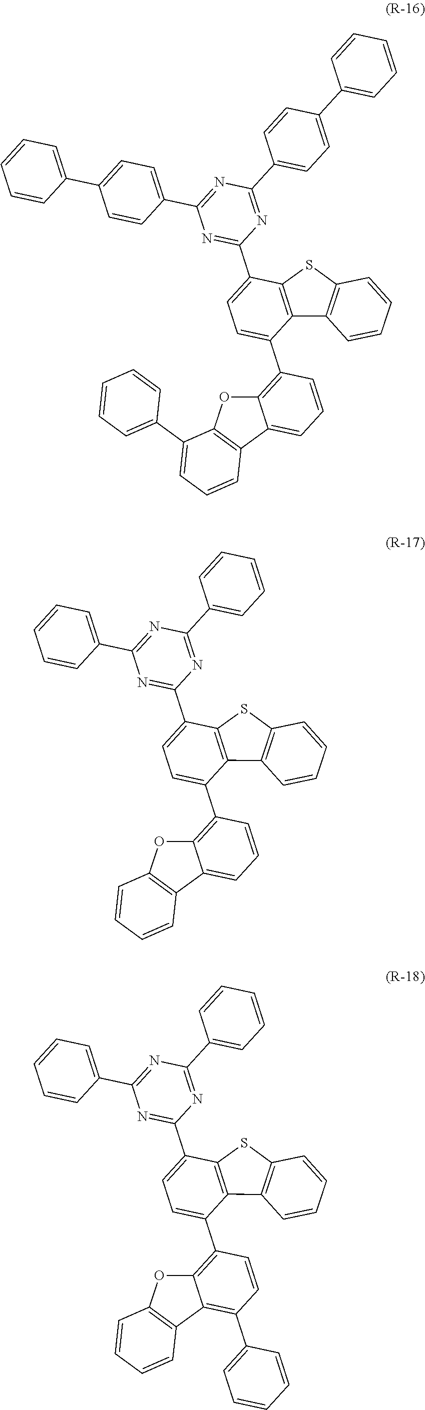 [SMILES: c1ccc(-c2ccc(-c3nc(-c4ccc(-c5ccccc5)cc4)nc(-c4ccc(-c5cccc6c5oc5c(-c7ccccc7)cccc56)c5c4sc4ccccc45)n3)cc2)cc1 ; c1ccc(-c2nc(-c3ccccc3)nc(-c3ccc(-c4cccc5c4oc4ccccc45)c4c3sc3ccccc34)n2)cc1 ; c1ccc(-c2nc(-c3ccccc3)nc(-c3ccc(-c4ccc(-c5ccccc5)c5c4oc4ccccc45)c4c3sc3ccccc34)n2)cc1]

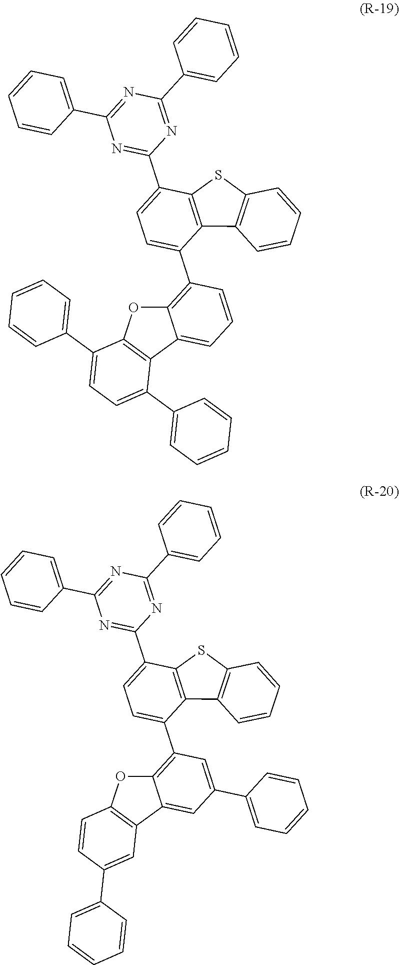

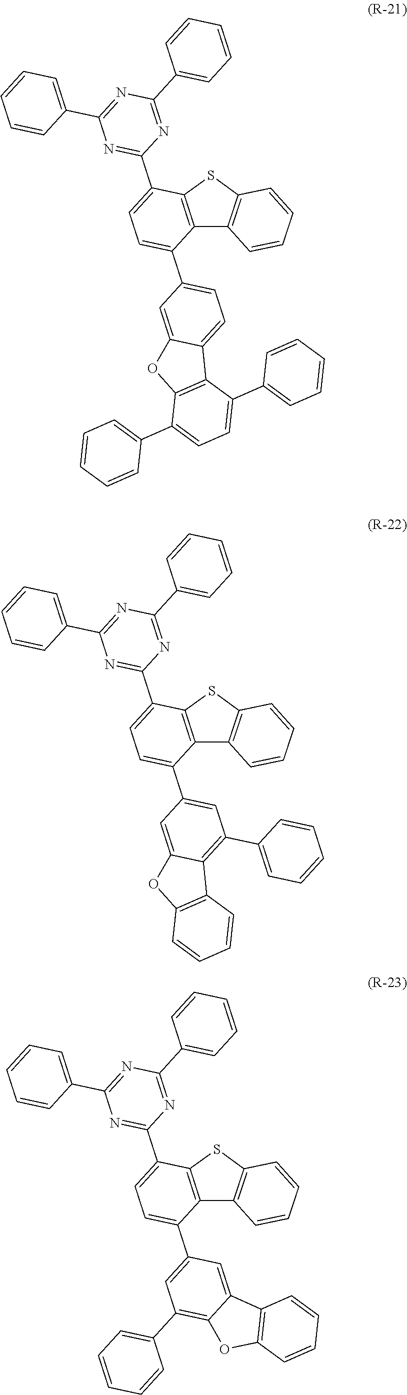

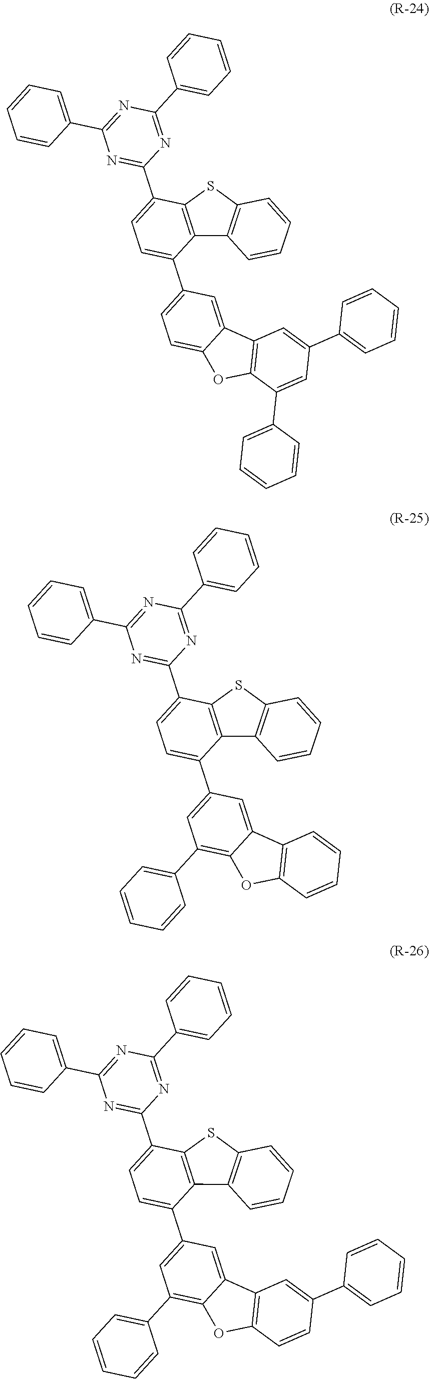 [SMILES: c1ccc(-c2cc(-c3ccccc3)c3oc4ccc(-c5ccc(-c6nc(-c7ccccc7)nc(-c7ccccc7)n6)c6sc7ccccc7c56)cc4c3c2)cc1 ; c1ccc(-c2nc(-c3ccccc3)nc(-c3ccc(-c4cc(-c5ccccc5)c5oc6ccccc6c5c4)c4c3sc3ccccc34)n2)cc1 ; c1ccc(-c2ccc3oc4c(-c5ccccc5)cc(-c5ccc(-c6nc(-c7ccccc7)nc(-c7ccccc7)n6)c6sc7ccccc7c56)cc4c3c2)cc1]

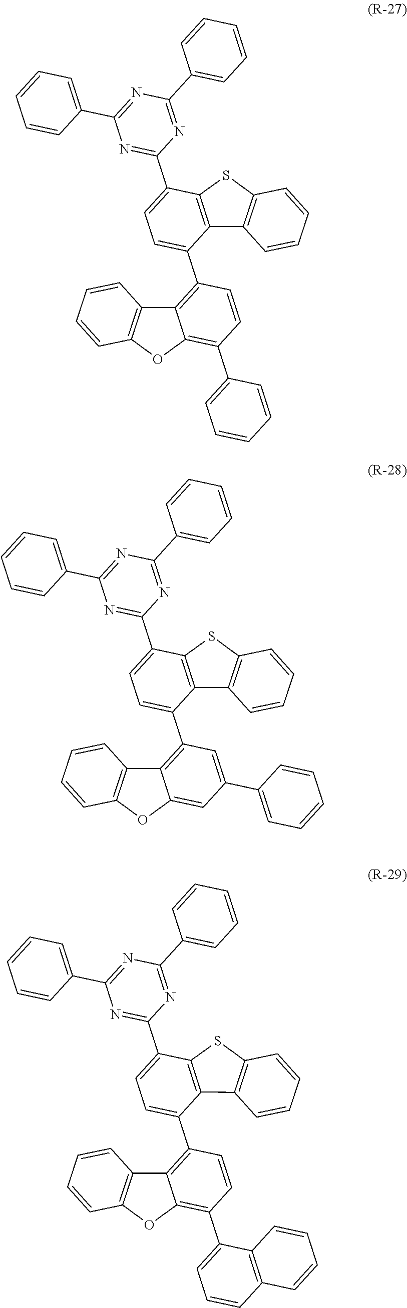 [SMILES: c1ccc(-c2nc(-c3ccccc3)nc(-c3ccc(-c4ccc(-c5ccccc5)c5oc6ccccc6c45)c4c3sc3ccccc34)n2)cc1 ; c1ccc(-c2cc(-c3ccc(-c4nc(-c5ccccc5)nc(-c5ccccc5)n4)c4sc5ccccc5c34)c3c(c2)oc2ccccc23)cc1 ; c1ccc(-c2nc(-c3ccccc3)nc(-c3ccc(-c4ccc(-c5cccc6ccccc56)c5oc6ccccc6c45)c4c3sc3ccccc34)n2)cc1]

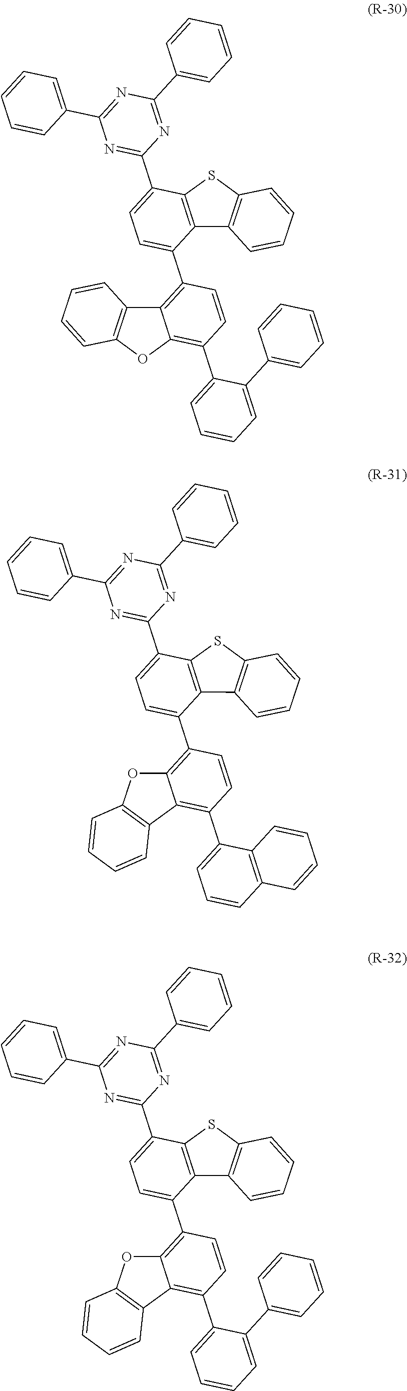 [SMILES: c1ccc(-c2nc(-c3ccccc3)nc(-c3ccc(-c4ccc(-c5ccccc5-c5ccccc5)c5oc6ccccc6c45)c4c3sc3ccccc34)n2)cc1 ; c1ccc(-c2nc(-c3ccccc3)nc(-c3ccc(-c4ccc(-c5cccc6ccccc56)c5c4oc4ccccc45)c4c3sc3ccccc34)n2)cc1 ; c1ccc(-c2nc(-c3ccccc3)nc(-c3ccc(-c4ccc(-c5ccccc5-c5ccccc5)c5c4oc4ccccc45)c4c3sc3ccccc34)n2)cc1]

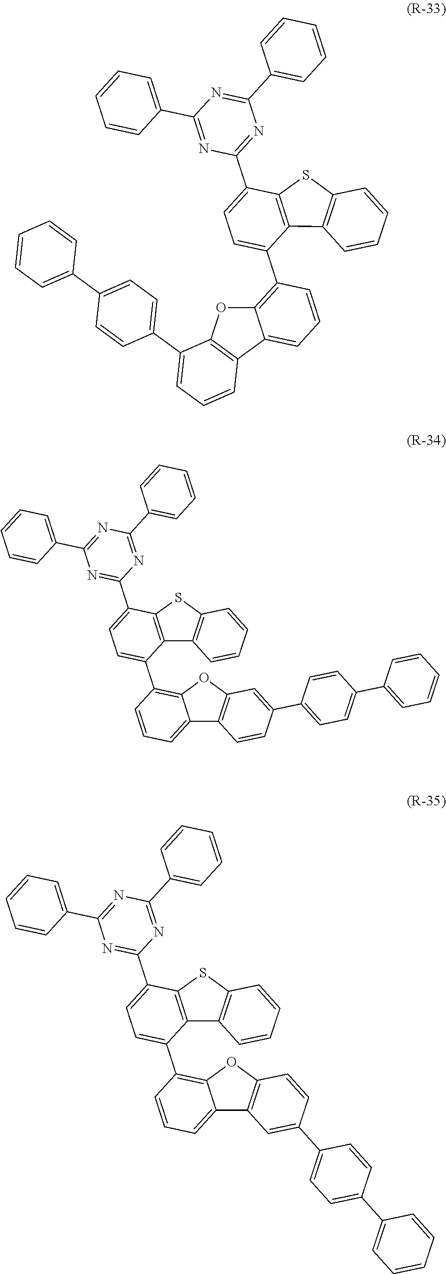 [SMILES: c1ccc(-c2ccc(-c3cccc4c3oc3c(-c5ccc(-c6nc(-c7ccccc7)nc(-c7ccccc7)n6)c6sc7ccccc7c56)cccc34)cc2)cc1 ; c1ccc(-c2ccc(-c3ccc4c(c3)oc3c(-c5ccc(-c6nc(-c7ccccc7)nc(-c7ccccc7)n6)c6sc7ccccc7c56)cccc34)cc2)cc1 ; c1ccc(-c2ccc(-c3ccc4oc5c(-c6ccc(-c7nc(-c8ccccc8)nc(-c8ccccc8)n7)c7sc8ccccc8c67)cccc5c4c3)cc2)cc1]

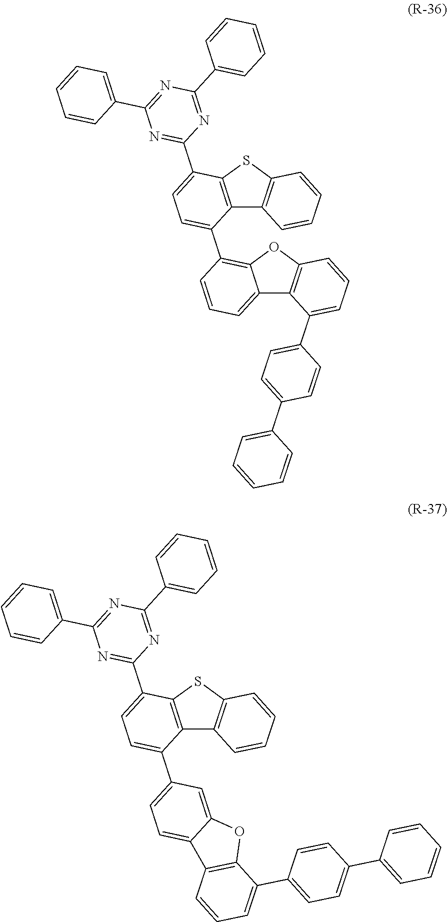 [SMILES: c1ccc(-c2ccc(-c3cccc4oc5c(-c6ccc(-c7nc(-c8ccccc8)nc(-c8ccccc8)n7)c7sc8ccccc8c67)cccc5c34)cc2)cc1 ; c1ccc(-c2ccc(-c3cccc4c3oc3cc(-c5ccc(-c6nc(-c7ccccc7)nc(-c7ccccc7)n6)c6sc7ccccc7c56)ccc34)cc2)cc1]

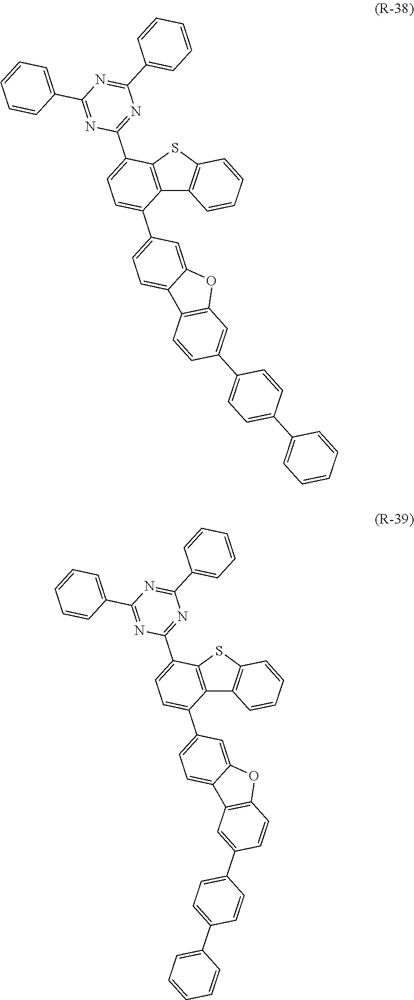 [SMILES: c1ccc(-c2ccc(-c3ccc4c(c3)oc3cc(-c5ccc(-c6nc(-c7ccccc7)nc(-c7ccccc7)n6)c6sc7ccccc7c56)ccc34)cc2)cc1 ; c1ccc(-c2ccc(-c3ccc4oc5cc(-c6ccc(-c7nc(-c8ccccc8)nc(-c8ccccc8)n7)c7sc8ccccc8c67)ccc5c4c3)cc2)cc1]

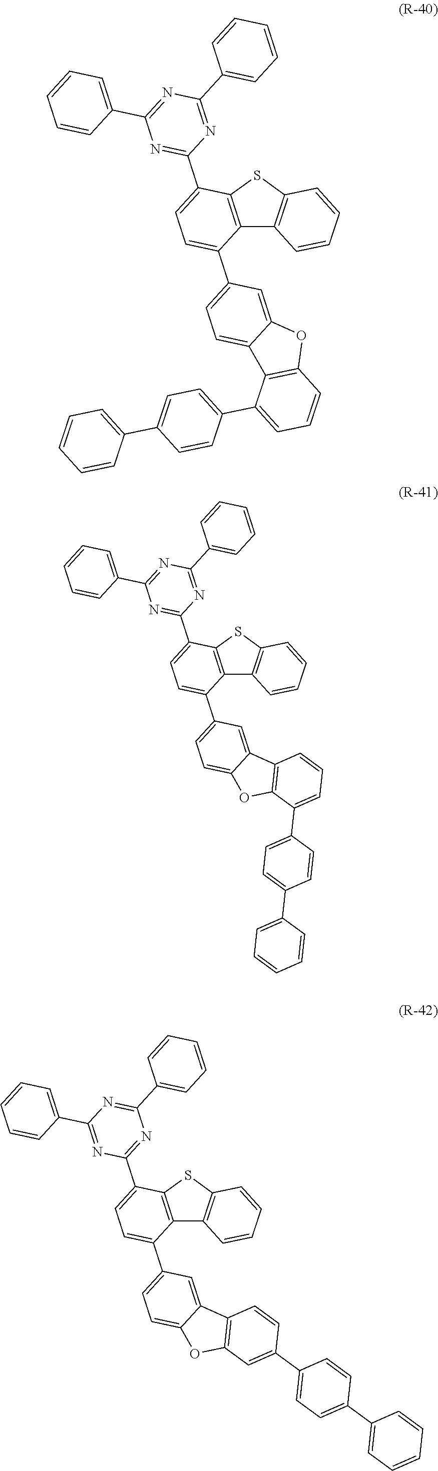 [SMILES: c1ccc(-c2ccc(-c3cccc4oc5cc(-c6ccc(-c7nc(-c8ccccc8)nc(-c8ccccc8)n7)c7sc8ccccc8c67)ccc5c34)cc2)cc1 ; c1ccc(-c2ccc(-c3cccc4c3oc3ccc(-c5ccc(-c6nc(-c7ccccc7)nc(-c7ccccc7)n6)c6sc7ccccc7c56)cc34)cc2)cc1 ; c1ccc(-c2ccc(-c3ccc4c(c3)oc3ccc(-c5ccc(-c6nc(-c7ccccc7)nc(-c7ccccc7)n6)c6sc7ccccc7c56)cc34)cc2)cc1]

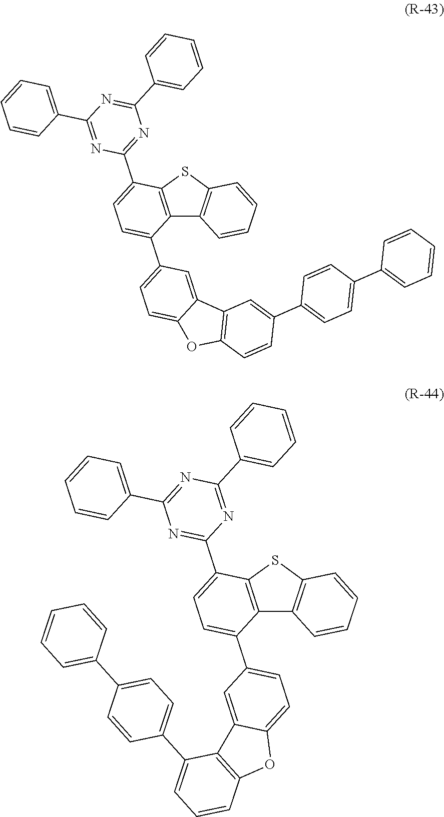 [SMILES: c1ccc(-c2ccc(-c3ccc4oc5ccc(-c6ccc(-c7nc(-c8ccccc8)nc(-c8ccccc8)n7)c7sc8ccccc8c67)cc5c4c3)cc2)cc1 ; c1ccc(-c2ccc(-c3cccc4oc5ccc(-c6ccc(-c7nc(-c8ccccc8)nc(-c8ccccc8)n7)c7sc8ccccc8c67)cc5c34)cc2)cc1]

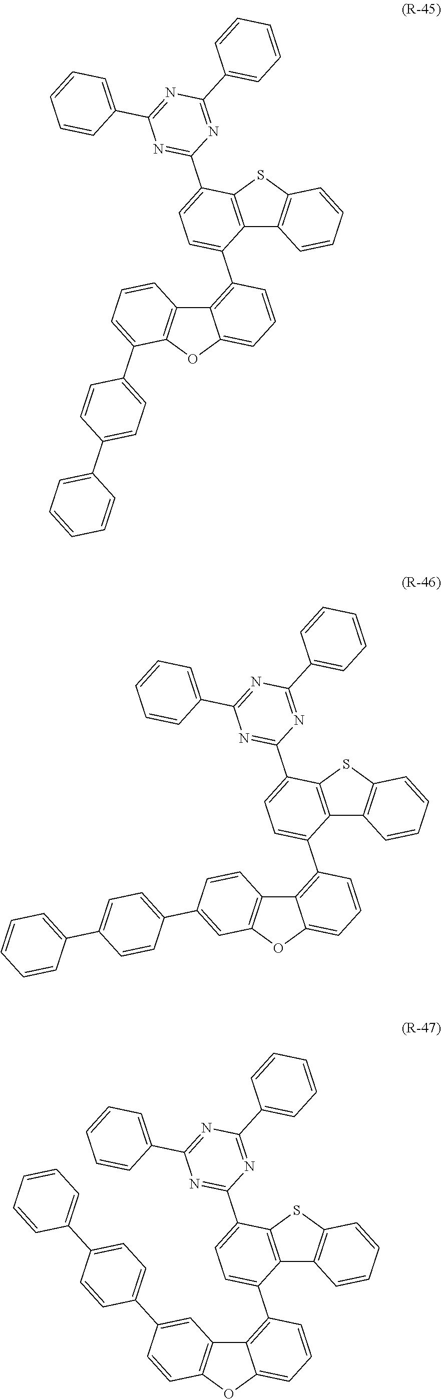 [SMILES: c1ccc(-c2ccc(-c3cccc4c3oc3cccc(-c5ccc(-c6nc(-c7ccccc7)nc(-c7ccccc7)n6)c6sc7ccccc7c56)c34)cc2)cc1 ; c1ccc(-c2ccc(-c3ccc4c(c3)oc3cccc(-c5ccc(-c6nc(-c7ccccc7)nc(-c7ccccc7)n6)c6sc7ccccc7c56)c34)cc2)cc1 ; c1ccc(-c2ccc(-c3ccc4oc5cccc(-c6ccc(-c7nc(-c8ccccc8)nc(-c8ccccc8)n7)c7sc8ccccc8c67)c5c4c3)cc2)cc1]

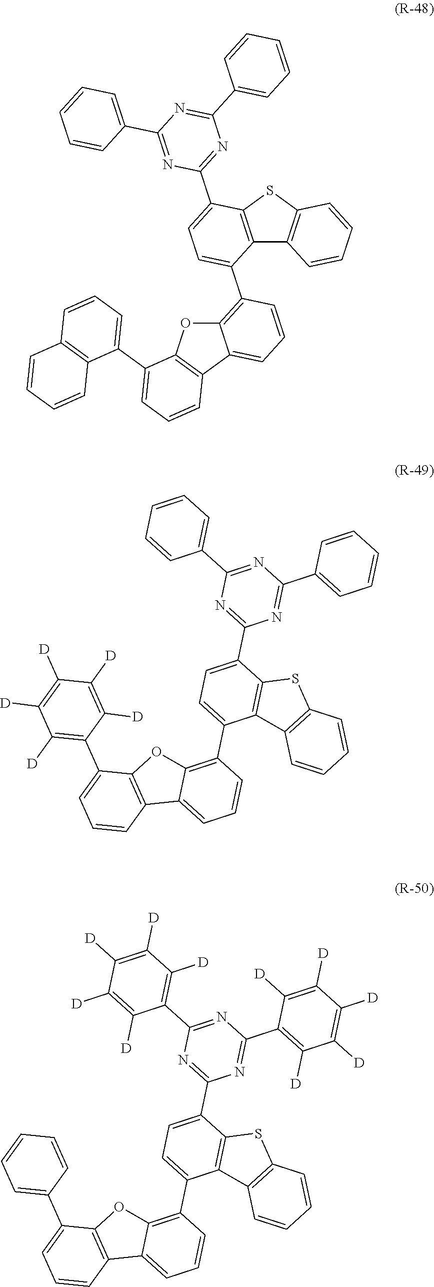 [SMILES: c1ccc(-c2nc(-c3ccccc3)nc(-c3ccc(-c4cccc5c4oc4c(-c6cccc7ccccc67)cccc45)c4c3sc3ccccc34)n2)cc1 ; [2H]c1c([2H])c([2H])c(-c2cccc3c2oc2c(-c4ccc(-c5nc(-c6ccccc6)nc(-c6ccccc6)n5)c5sc6ccccc6c45)cccc23)c([2H])c1[2H] ; [2H]c1c([2H])c([2H])c(-c2nc(-c3c([2H])c([2H])c([2H])c([2H])c3[2H])nc(-c3ccc(-c4cccc5c4oc4c(-c6ccccc6)cccc45)c4c3sc3ccccc34)n2)c([2H])c1[2H]]

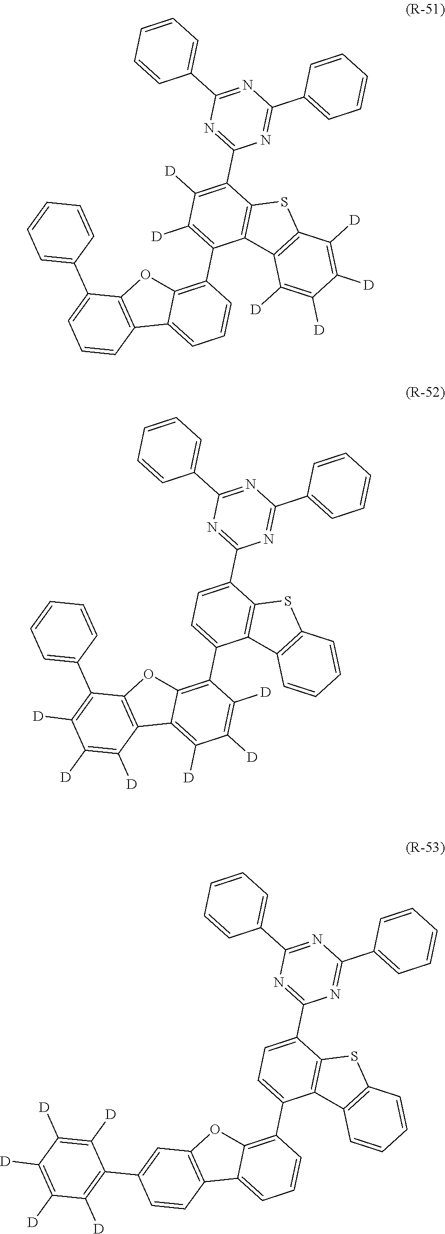 [SMILES: [2H]c1c([2H])c([2H])c2c(sc3c(-c4nc(-c5ccccc5)nc(-c5ccccc5)n4)c([2H])c([2H])c(-c4cccc5c4oc4c(-c6ccccc6)cccc45)c32)c1[2H] ; [2H]c1c([2H])c([2H])c2c(oc3c(-c4ccc(-c5nc(-c6ccccc6)nc(-c6ccccc6)n5)c5sc6ccccc6c45)c([2H])c([2H])c([2H])c32)c1-c1ccccc1 ; [2H]c1c([2H])c([2H])c(-c2ccc3c(c2)oc2c(-c4ccc(-c5nc(-c6ccccc6)nc(-c6ccccc6)n5)c5sc6ccccc6c45)cccc23)c([2H])c1[2H]]

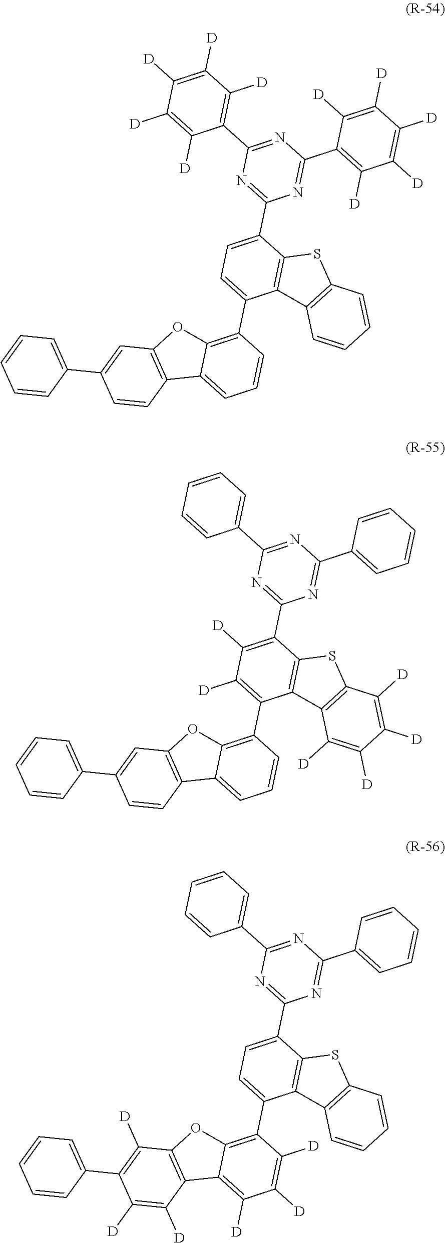 [SMILES: [2H]c1c([2H])c([2H])c(-c2nc(-c3c([2H])c([2H])c([2H])c([2H])c3[2H])nc(-c3ccc(-c4cccc5c4oc4cc(-c6ccccc6)ccc45)c4c3sc3ccccc34)n2)c([2H])c1[2H] ; [2H]c1c([2H])c([2H])c2c(sc3c(-c4nc(-c5ccccc5)nc(-c5ccccc5)n4)c([2H])c([2H])c(-c4cccc5c4oc4cc(-c6ccccc6)ccc45)c32)c1[2H] ; [2H]c1c([2H])c([2H])c2c(oc3c([2H])c(-c4ccccc4)c([2H])c([2H])c32)c1-c1ccc(-c2nc(-c3ccccc3)nc(-c3ccccc3)n2)c2sc3ccccc3c12]

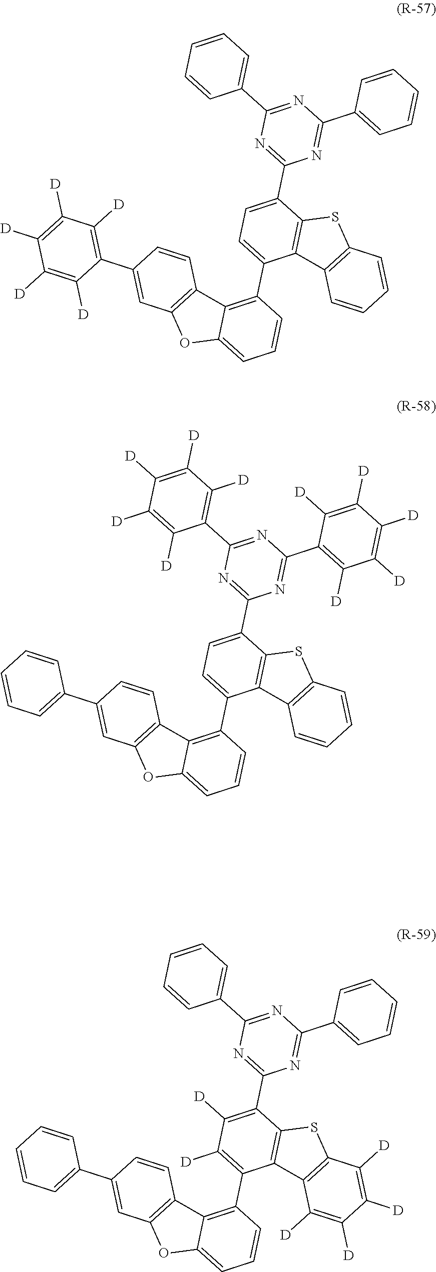 [SMILES: [2H]c1c([2H])c([2H])c(-c2ccc3c(c2)oc2cccc(-c4ccc(-c5nc(-c6ccccc6)nc(-c6ccccc6)n5)c5sc6ccccc6c45)c23)c([2H])c1[2H] ; [2H]c1c([2H])c([2H])c(-c2nc(-c3c([2H])c([2H])c([2H])c([2H])c3[2H])nc(-c3ccc(-c4cccc5oc6cc(-c7ccccc7)ccc6c45)c4c3sc3ccccc34)n2)c([2H])c1[2H] ; [2H]c1c([2H])c([2H])c2c(sc3c(-c4nc(-c5ccccc5)nc(-c5ccccc5)n4)c([2H])c([2H])c(-c4cccc5oc6cc(-c7ccccc7)ccc6c45)c32)c1[2H]]

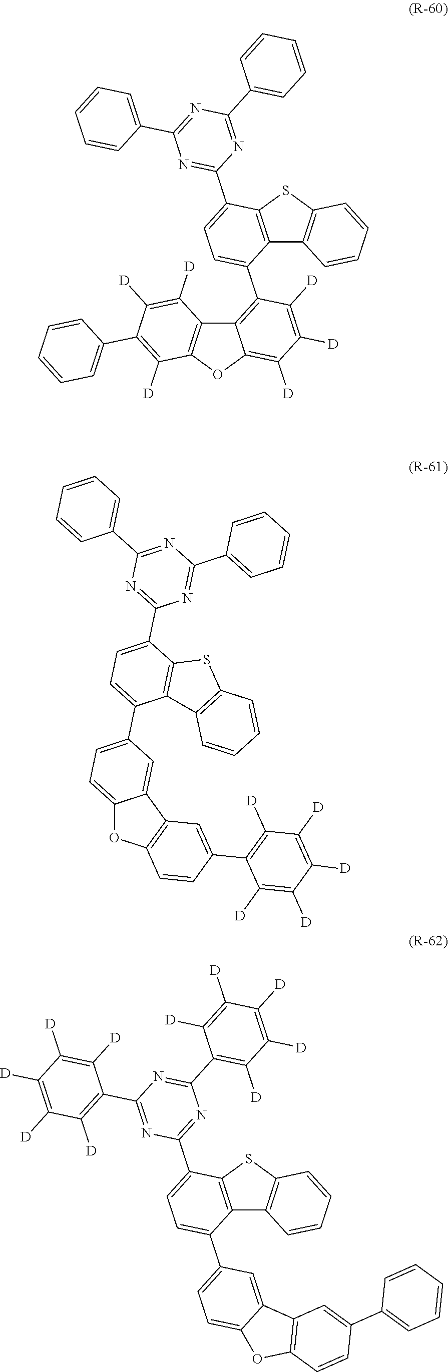 [SMILES: [2H]c1c([2H])c(-c2ccc(-c3nc(-c4ccccc4)nc(-c4ccccc4)n3)c3sc4ccccc4c23)c2c(oc3c([2H])c(-c4ccccc4)c([2H])c([2H])c32)c1[2H] ; [2H]c1c([2H])c([2H])c(-c2ccc3oc4ccc(-c5ccc(-c6nc(-c7ccccc7)nc(-c7ccccc7)n6)c6sc7ccccc7c56)cc4c3c2)c([2H])c1[2H] ; [2H]c1c([2H])c([2H])c(-c2nc(-c3c([2H])c([2H])c([2H])c([2H])c3[2H])nc(-c3ccc(-c4ccc5oc6ccc(-c7ccccc7)cc6c5c4)c4c3sc3ccccc34)n2)c([2H])c1[2H]]

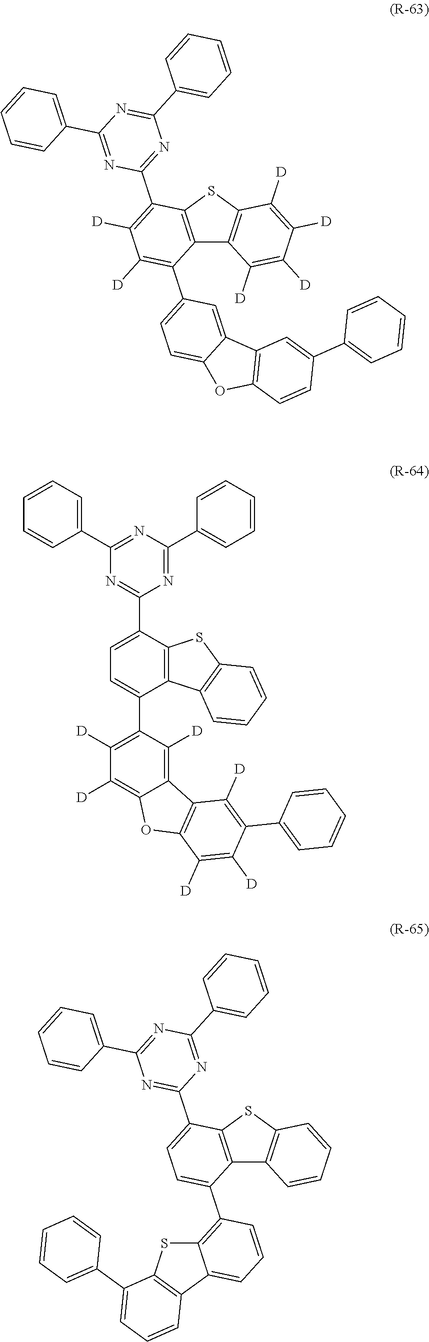 [SMILES: [2H]c1c([2H])c([2H])c2c(sc3c(-c4nc(-c5ccccc5)nc(-c5ccccc5)n4)c([2H])c([2H])c(-c4ccc5oc6ccc(-c7ccccc7)cc6c5c4)c32)c1[2H] ; [2H]c1c(-c2ccccc2)c([2H])c2c(oc3c([2H])c([2H])c(-c4ccc(-c5nc(-c6ccccc6)nc(-c6ccccc6)n5)c5sc6ccccc6c45)c([2H])c32)c1[2H] ; c1ccc(-c2nc(-c3ccccc3)nc(-c3ccc(-c4cccc5c4sc4c(-c6ccccc6)cccc45)c4c3sc3ccccc34)n2)cc1]

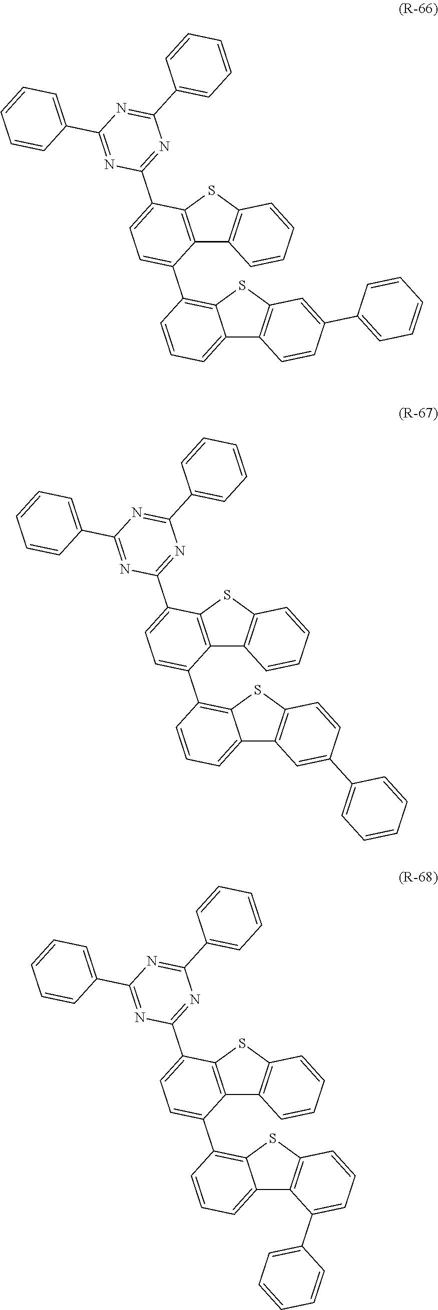 [SMILES: c1ccc(-c2ccc3c(c2)sc2c(-c4ccc(-c5nc(-c6ccccc6)nc(-c6ccccc6)n5)c5sc6ccccc6c45)cccc23)cc1 ; c1ccc(-c2ccc3sc4c(-c5ccc(-c6nc(-c7ccccc7)nc(-c7ccccc7)n6)c6sc7ccccc7c56)cccc4c3c2)cc1 ; c1ccc(-c2nc(-c3ccccc3)nc(-c3ccc(-c4cccc5c4sc4cccc(-c6ccccc6)c45)c4c3sc3ccccc34)n2)cc1]

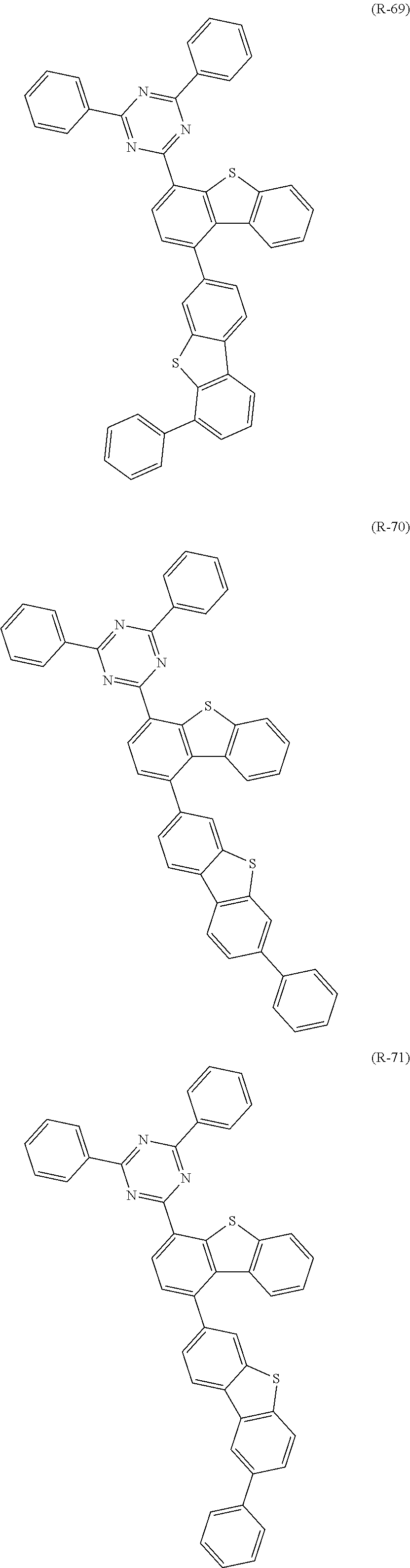 [SMILES: c1ccc(-c2nc(-c3ccccc3)nc(-c3ccc(-c4ccc5c(c4)sc4c(-c6ccccc6)cccc45)c4c3sc3ccccc34)n2)cc1 ; c1ccc(-c2ccc3c(c2)sc2cc(-c4ccc(-c5nc(-c6ccccc6)nc(-c6ccccc6)n5)c5sc6ccccc6c45)ccc23)cc1 ; c1ccc(-c2ccc3sc4cc(-c5ccc(-c6nc(-c7ccccc7)nc(-c7ccccc7)n6)c6sc7ccccc7c56)ccc4c3c2)cc1]

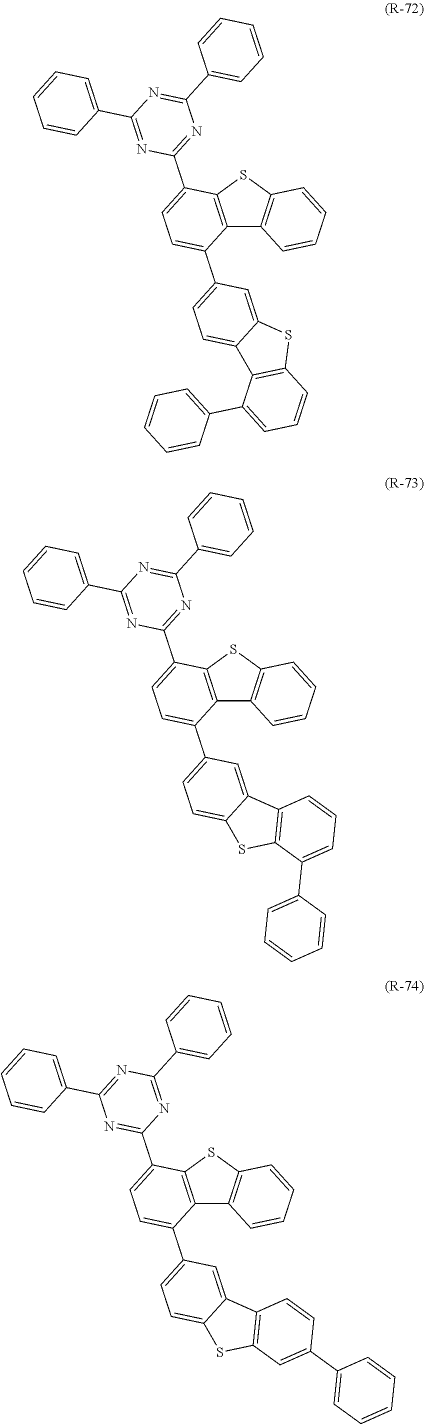 [SMILES: c1ccc(-c2nc(-c3ccccc3)nc(-c3ccc(-c4ccc5c(c4)sc4cccc(-c6ccccc6)c45)c4c3sc3ccccc34)n2)cc1 ; c1ccc(-c2nc(-c3ccccc3)nc(-c3ccc(-c4ccc5sc6c(-c7ccccc7)cccc6c5c4)c4c3sc3ccccc34)n2)cc1 ; c1ccc(-c2ccc3c(c2)sc2ccc(-c4ccc(-c5nc(-c6ccccc6)nc(-c6ccccc6)n5)c5sc6ccccc6c45)cc23)cc1]

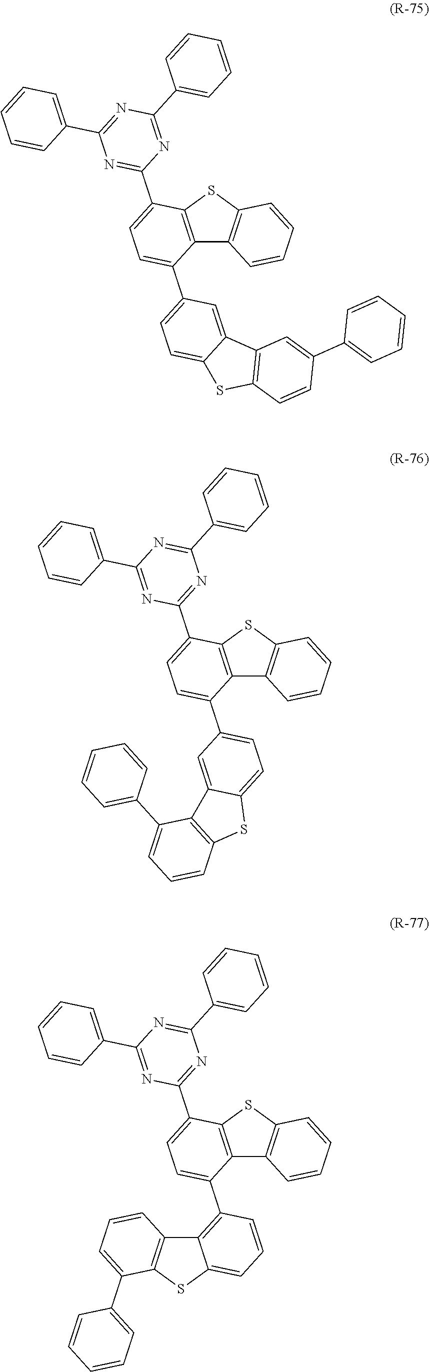 [SMILES: c1ccc(-c2ccc3sc4ccc(-c5ccc(-c6nc(-c7ccccc7)nc(-c7ccccc7)n6)c6sc7ccccc7c56)cc4c3c2)cc1 ; c1ccc(-c2nc(-c3ccccc3)nc(-c3ccc(-c4ccc5sc6cccc(-c7ccccc7)c6c5c4)c4c3sc3ccccc34)n2)cc1 ; c1ccc(-c2nc(-c3ccccc3)nc(-c3ccc(-c4cccc5sc6c(-c7ccccc7)cccc6c45)c4c3sc3ccccc34)n2)cc1]

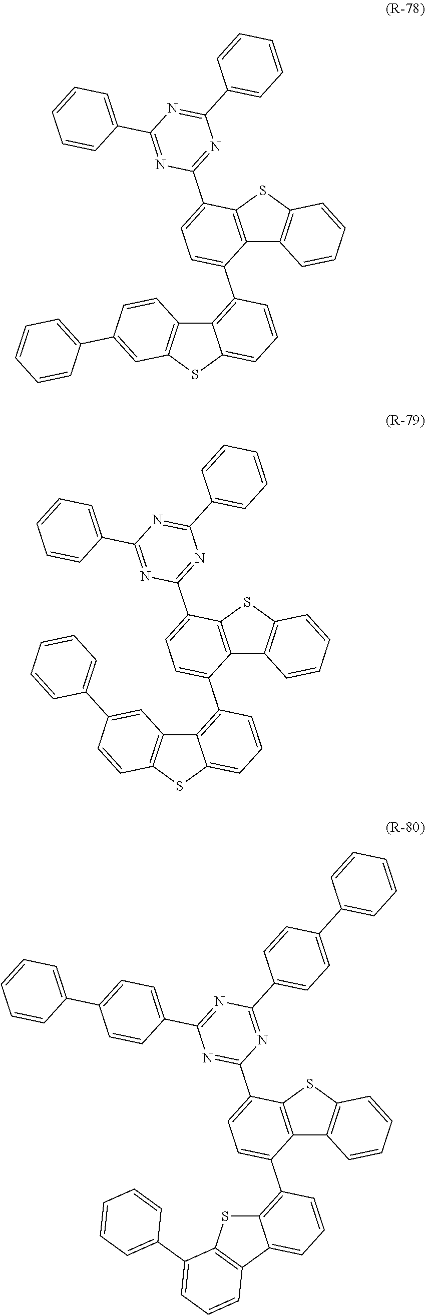 [SMILES: c1ccc(-c2ccc3c(c2)sc2cccc(-c4ccc(-c5nc(-c6ccccc6)nc(-c6ccccc6)n5)c5sc6ccccc6c45)c23)cc1 ; c1ccc(-c2ccc3sc4cccc(-c5ccc(-c6nc(-c7ccccc7)nc(-c7ccccc7)n6)c6sc7ccccc7c56)c4c3c2)cc1 ; c1ccc(-c2ccc(-c3nc(-c4ccc(-c5ccccc5)cc4)nc(-c4ccc(-c5cccc6c5sc5c(-c7ccccc7)cccc56)c5c4sc4ccccc45)n3)cc2)cc1]

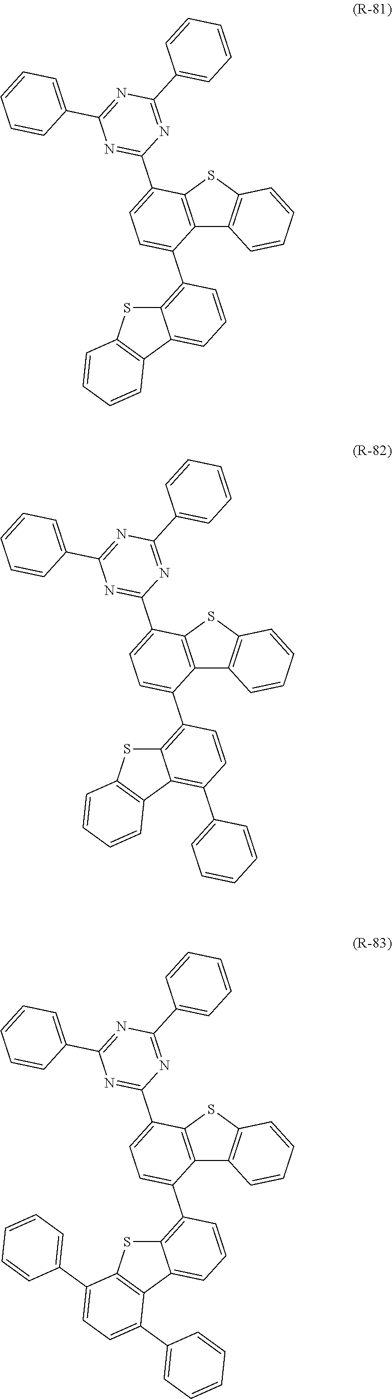 [SMILES: c1ccc(-c2nc(-c3ccccc3)nc(-c3ccc(-c4cccc5c4sc4ccccc45)c4c3sc3ccccc34)n2)cc1 ; c1ccc(-c2nc(-c3ccccc3)nc(-c3ccc(-c4ccc(-c5ccccc5)c5c4sc4ccccc45)c4c3sc3ccccc34)n2)cc1 ; c1ccc(-c2nc(-c3ccccc3)nc(-c3ccc(-c4cccc5c4sc4c(-c6ccccc6)ccc(-c6ccccc6)c45)c4c3sc3ccccc34)n2)cc1]

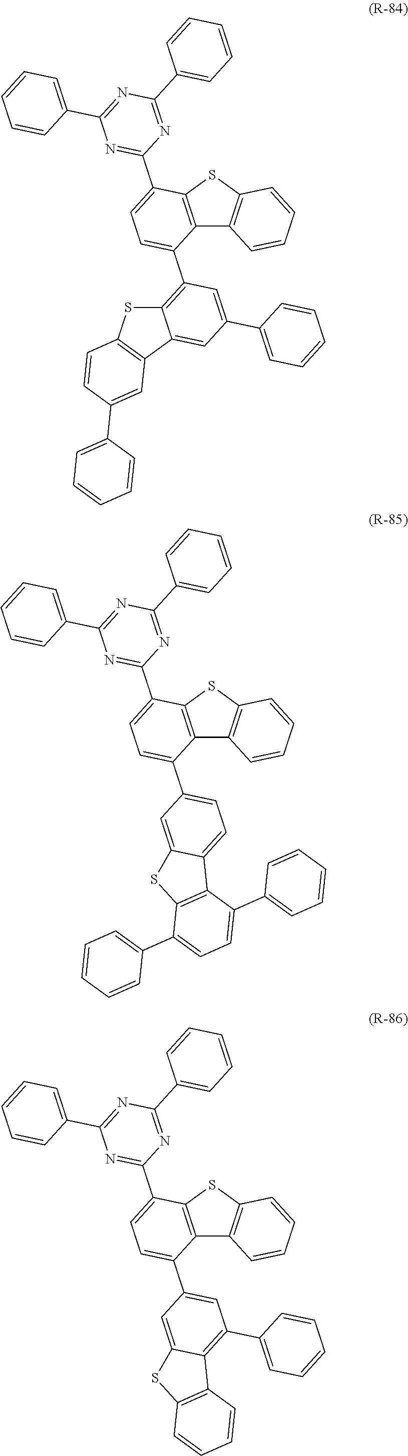 [SMILES: c1ccc(-c2ccc3sc4c(-c5ccc(-c6nc(-c7ccccc7)nc(-c7ccccc7)n6)c6sc7ccccc7c56)cc(-c5ccccc5)cc4c3c2)cc1 ; c1ccc(-c2nc(-c3ccccc3)nc(-c3ccc(-c4ccc5c(c4)sc4c(-c6ccccc6)ccc(-c6ccccc6)c45)c4c3sc3ccccc34)n2)cc1 ; c1ccc(-c2nc(-c3ccccc3)nc(-c3ccc(-c4cc(-c5ccccc5)c5c(c4)sc4ccccc45)c4c3sc3ccccc34)n2)cc1]

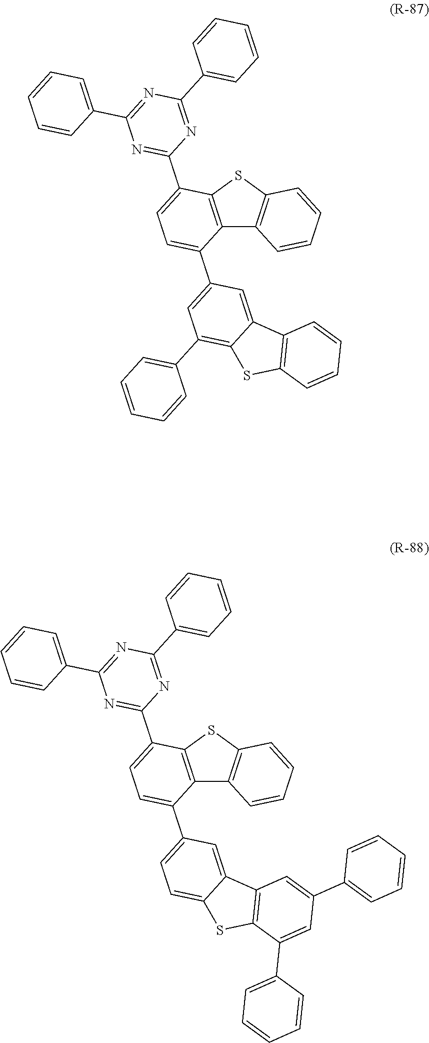 [SMILES: c1ccc(-c2nc(-c3ccccc3)nc(-c3ccc(-c4cc(-c5ccccc5)c5sc6ccccc6c5c4)c4c3sc3ccccc34)n2)cc1 ; c1ccc(-c2cc(-c3ccccc3)c3sc4ccc(-c5ccc(-c6nc(-c7ccccc7)nc(-c7ccccc7)n6)c6sc7ccccc7c56)cc4c3c2)cc1]

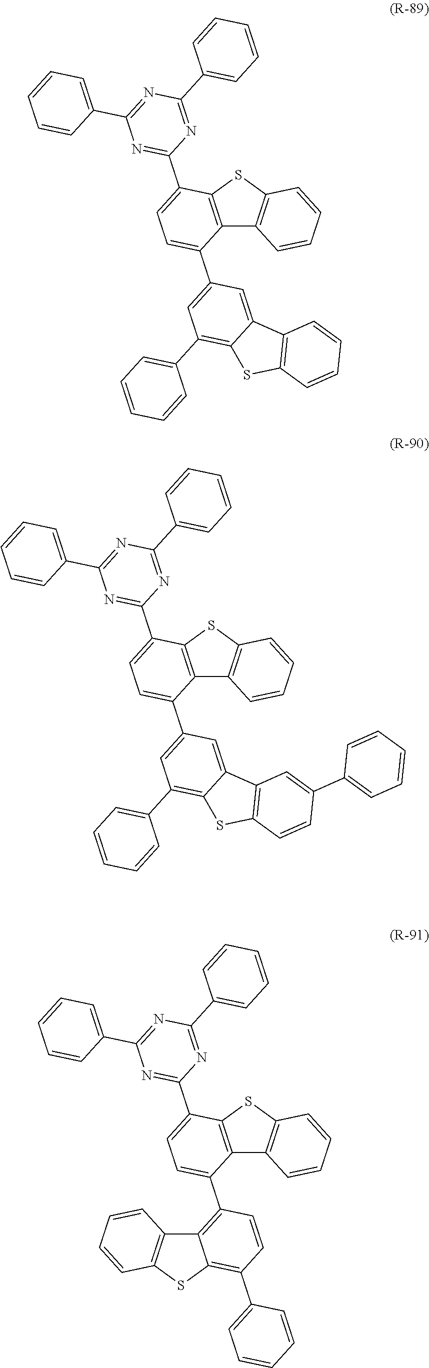 [SMILES: c1ccc(-c2nc(-c3ccccc3)nc(-c3ccc(-c4cc(-c5ccccc5)c5sc6ccccc6c5c4)c4c3sc3ccccc34)n2)cc1 ; c1ccc(-c2ccc3sc4c(-c5ccccc5)cc(-c5ccc(-c6nc(-c7ccccc7)nc(-c7ccccc7)n6)c6sc7ccccc7c56)cc4c3c2)cc1 ; c1ccc(-c2nc(-c3ccccc3)nc(-c3ccc(-c4ccc(-c5ccccc5)c5sc6ccccc6c45)c4c3sc3ccccc34)n2)cc1]

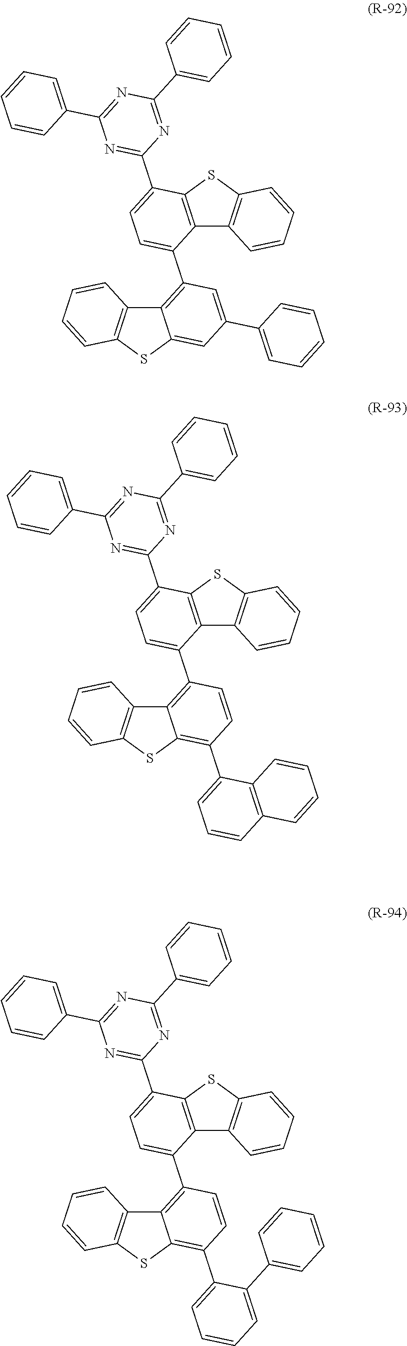 [SMILES: c1ccc(-c2cc(-c3ccc(-c4nc(-c5ccccc5)nc(-c5ccccc5)n4)c4sc5ccccc5c34)c3c(c2)sc2ccccc23)cc1 ; c1ccc(-c2nc(-c3ccccc3)nc(-c3ccc(-c4ccc(-c5cccc6ccccc56)c5sc6ccccc6c45)c4c3sc3ccccc34)n2)cc1 ; c1ccc(-c2nc(-c3ccccc3)nc(-c3ccc(-c4ccc(-c5ccccc5-c5ccccc5)c5sc6ccccc6c45)c4c3sc3ccccc34)n2)cc1]

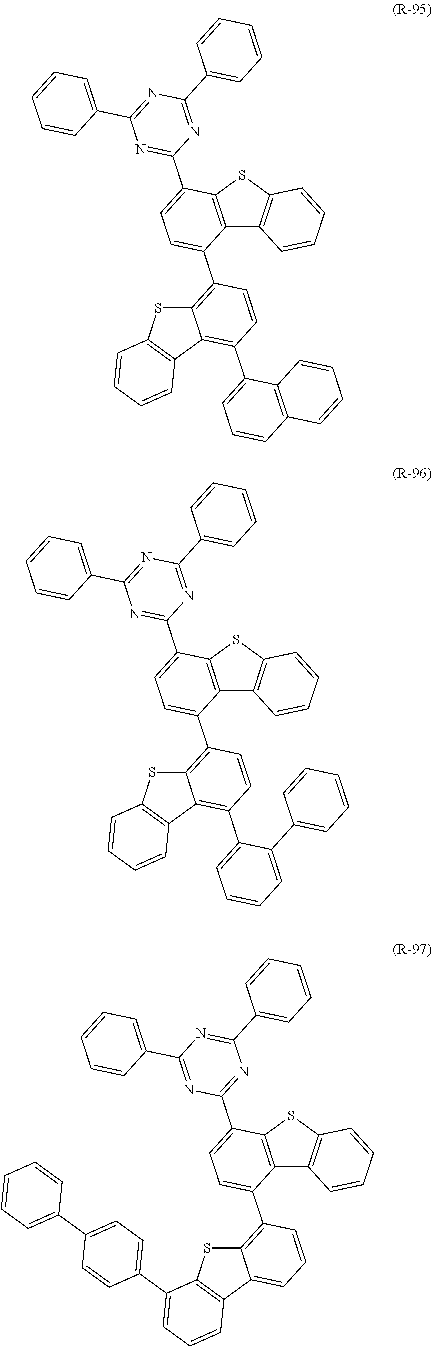 [SMILES: c1ccc(-c2nc(-c3ccccc3)nc(-c3ccc(-c4ccc(-c5cccc6ccccc56)c5c4sc4ccccc45)c4c3sc3ccccc34)n2)cc1 ; c1ccc(-c2nc(-c3ccccc3)nc(-c3ccc(-c4ccc(-c5ccccc5-c5ccccc5)c5c4sc4ccccc45)c4c3sc3ccccc34)n2)cc1 ; c1ccc(-c2ccc(-c3cccc4c3sc3c(-c5ccc(-c6nc(-c7ccccc7)nc(-c7ccccc7)n6)c6sc7ccccc7c56)cccc34)cc2)cc1]

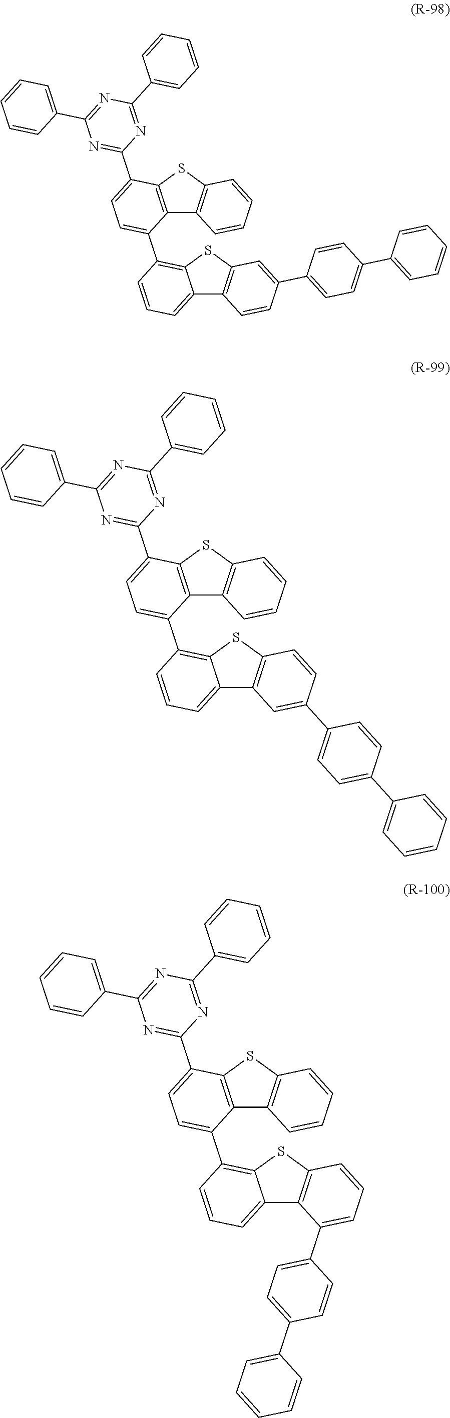 [SMILES: c1ccc(-c2ccc(-c3ccc4c(c3)sc3c(-c5ccc(-c6nc(-c7ccccc7)nc(-c7ccccc7)n6)c6sc7ccccc7c56)cccc34)cc2)cc1 ; c1ccc(-c2ccc(-c3ccc4sc5c(-c6ccc(-c7nc(-c8ccccc8)nc(-c8ccccc8)n7)c7sc8ccccc8c67)cccc5c4c3)cc2)cc1 ; c1ccc(-c2ccc(-c3cccc4sc5c(-c6ccc(-c7nc(-c8ccccc8)nc(-c8ccccc8)n7)c7sc8ccccc8c67)cccc5c34)cc2)cc1]

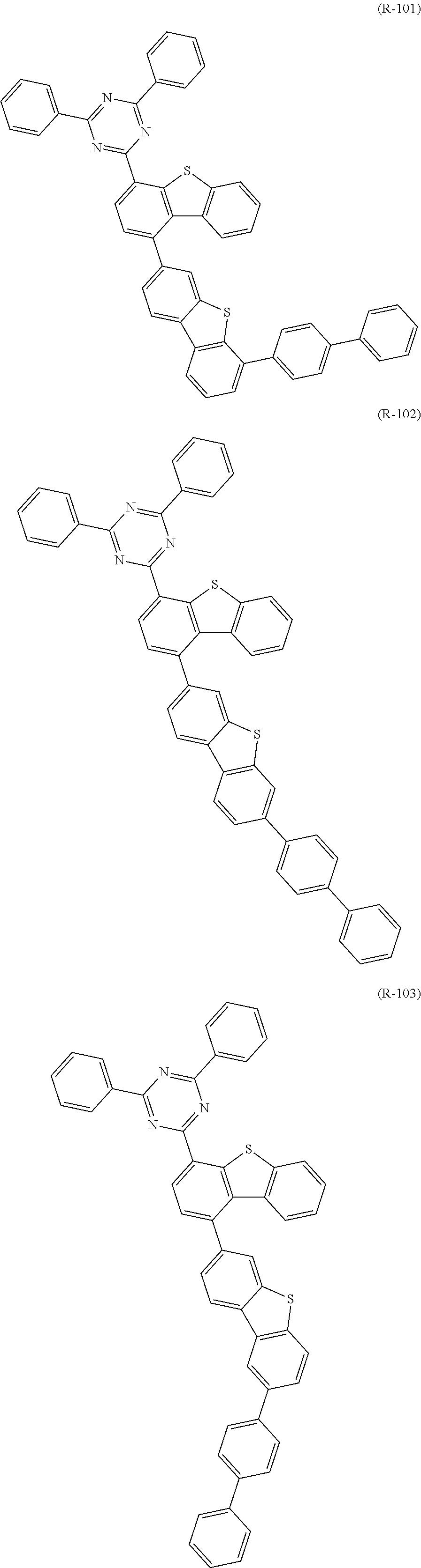

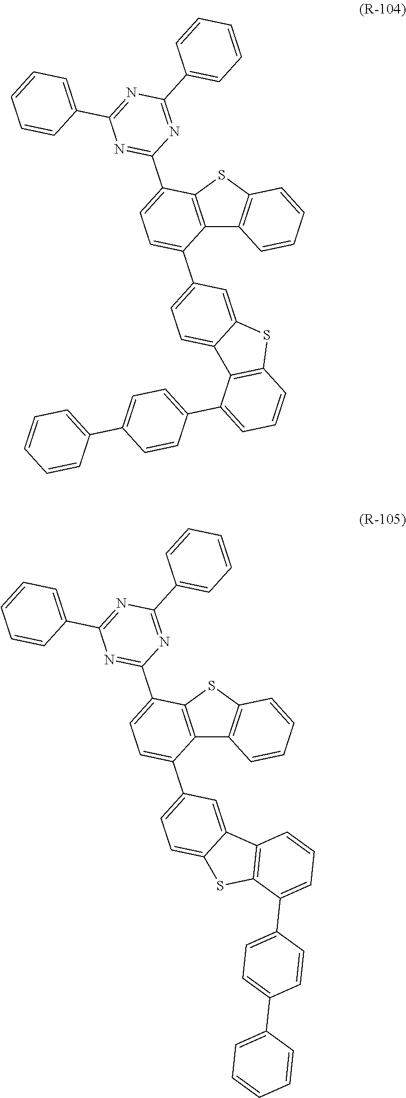 [SMILES: c1ccc(-c2ccc(-c3cccc4sc5cc(-c6ccc(-c7nc(-c8ccccc8)nc(-c8ccccc8)n7)c7sc8ccccc8c67)ccc5c34)cc2)cc1 ; c1ccc(-c2ccc(-c3cccc4c3sc3ccc(-c5ccc(-c6nc(-c7ccccc7)nc(-c7ccccc7)n6)c6sc7ccccc7c56)cc34)cc2)cc1]

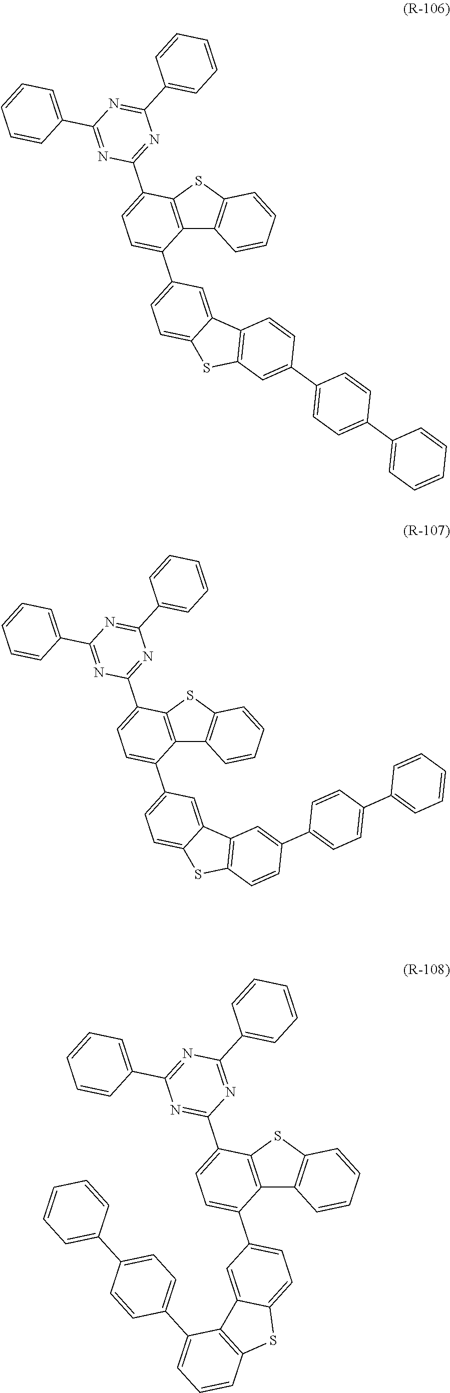 [SMILES: c1ccc(-c2ccc(-c3ccc4c(c3)sc3ccc(-c5ccc(-c6nc(-c7ccccc7)nc(-c7ccccc7)n6)c6sc7ccccc7c56)cc34)cc2)cc1 ; c1ccc(-c2ccc(-c3ccc4sc5ccc(-c6ccc(-c7nc(-c8ccccc8)nc(-c8ccccc8)n7)c7sc8ccccc8c67)cc5c4c3)cc2)cc1 ; c1ccc(-c2ccc(-c3cccc4sc5ccc(-c6ccc(-c7nc(-c8ccccc8)nc(-c8ccccc8)n7)c7sc8ccccc8c67)cc5c34)cc2)cc1]

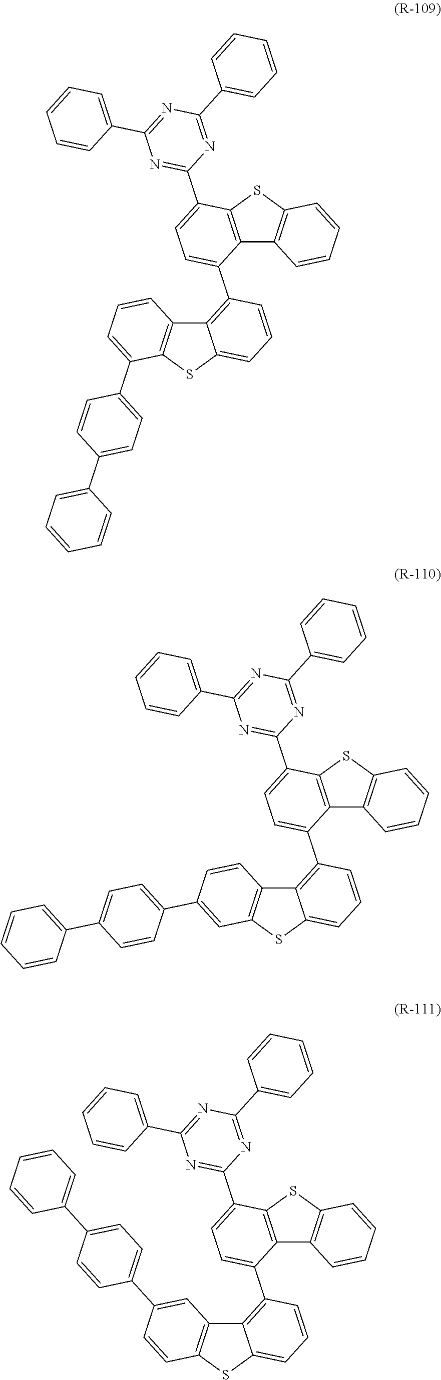 [SMILES: c1ccc(-c2ccc(-c3cccc4c3sc3cccc(-c5ccc(-c6nc(-c7ccccc7)nc(-c7ccccc7)n6)c6sc7ccccc7c56)c34)cc2)cc1 ; c1ccc(-c2ccc(-c3ccc4c(c3)sc3cccc(-c5ccc(-c6nc(-c7ccccc7)nc(-c7ccccc7)n6)c6sc7ccccc7c56)c34)cc2)cc1 ; c1ccc(-c2ccc(-c3ccc4sc5cccc(-c6ccc(-c7nc(-c8ccccc8)nc(-c8ccccc8)n7)c7sc8ccccc8c67)c5c4c3)cc2)cc1]

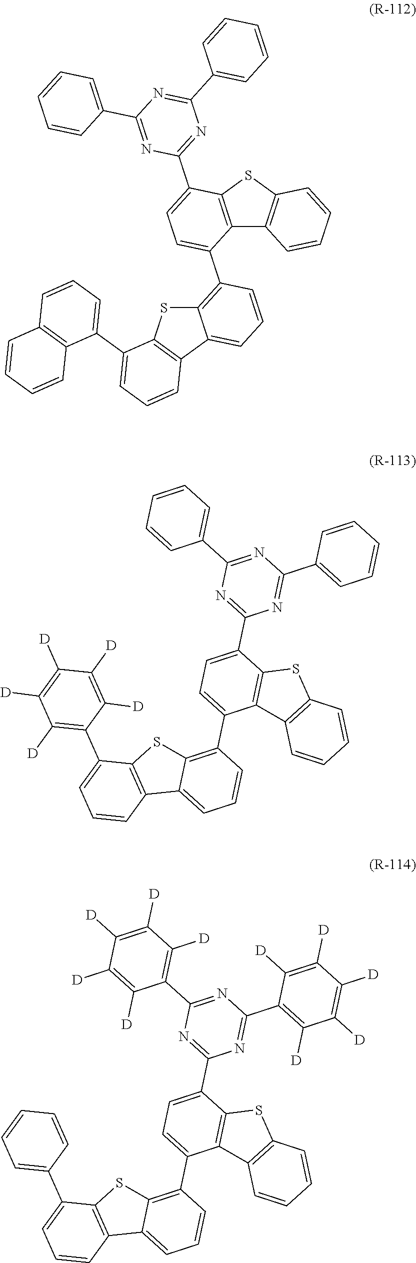 [SMILES: c1ccc(-c2nc(-c3ccccc3)nc(-c3ccc(-c4cccc5c4sc4c(-c6cccc7ccccc67)cccc45)c4c3sc3ccccc34)n2)cc1 ; [2H]c1c([2H])c([2H])c(-c2cccc3c2sc2c(-c4ccc(-c5nc(-c6ccccc6)nc(-c6ccccc6)n5)c5sc6ccccc6c45)cccc23)c([2H])c1[2H] ; [2H]c1c([2H])c([2H])c(-c2nc(-c3c([2H])c([2H])c([2H])c([2H])c3[2H])nc(-c3ccc(-c4cccc5c4sc4c(-c6ccccc6)cccc45)c4c3sc3ccccc34)n2)c([2H])c1[2H]]

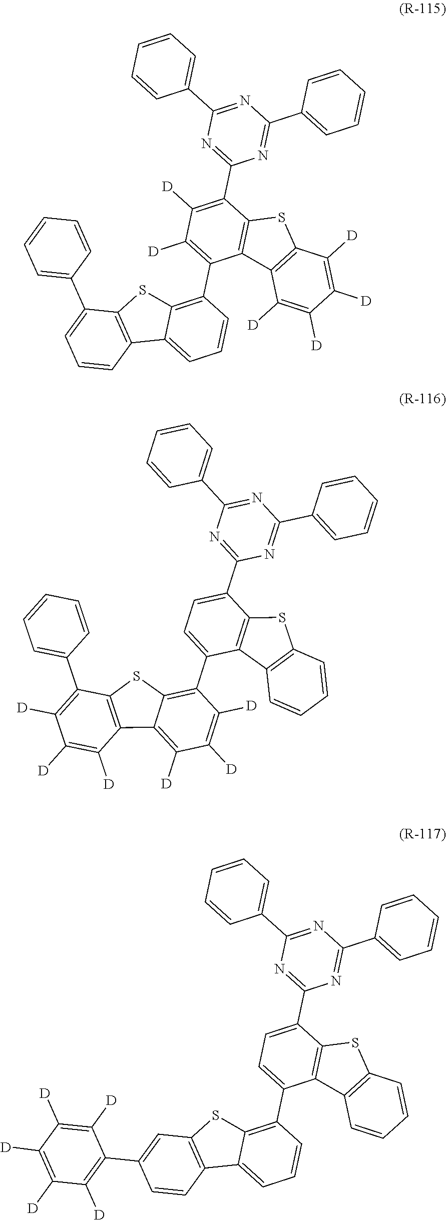 [SMILES: [2H]c1c([2H])c([2H])c2c(sc3c(-c4nc(-c5ccccc5)nc(-c5ccccc5)n4)c([2H])c([2H])c(-c4cccc5c4sc4c(-c6ccccc6)cccc45)c32)c1[2H] ; [2H]c1c([2H])c([2H])c2c(sc3c(-c4ccc(-c5nc(-c6ccccc6)nc(-c6ccccc6)n5)c5sc6ccccc6c45)c([2H])c([2H])c([2H])c32)c1-c1ccccc1 ; [2H]c1c([2H])c([2H])c(-c2ccc3c(c2)sc2c(-c4ccc(-c5nc(-c6ccccc6)nc(-c6ccccc6)n5)c5sc6ccccc6c45)cccc23)c([2H])c1[2H]]

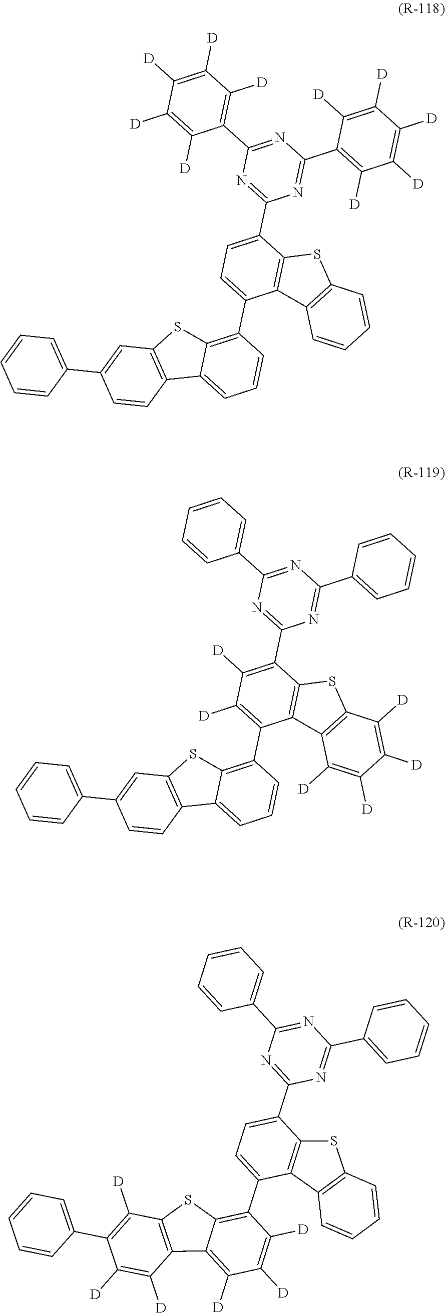 [SMILES: [2H]c1c([2H])c([2H])c(-c2nc(-c3c([2H])c([2H])c([2H])c([2H])c3[2H])nc(-c3ccc(-c4cccc5c4sc4cc(-c6ccccc6)ccc45)c4c3sc3ccccc34)n2)c([2H])c1[2H] ; [2H]c1c([2H])c([2H])c2c(sc3c(-c4nc(-c5ccccc5)nc(-c5ccccc5)n4)c([2H])c([2H])c(-c4cccc5c4sc4cc(-c6ccccc6)ccc45)c32)c1[2H] ; [2H]c1c([2H])c([2H])c2c(sc3c([2H])c(-c4ccccc4)c([2H])c([2H])c32)c1-c1ccc(-c2nc(-c3ccccc3)nc(-c3ccccc3)n2)c2sc3ccccc3c12]

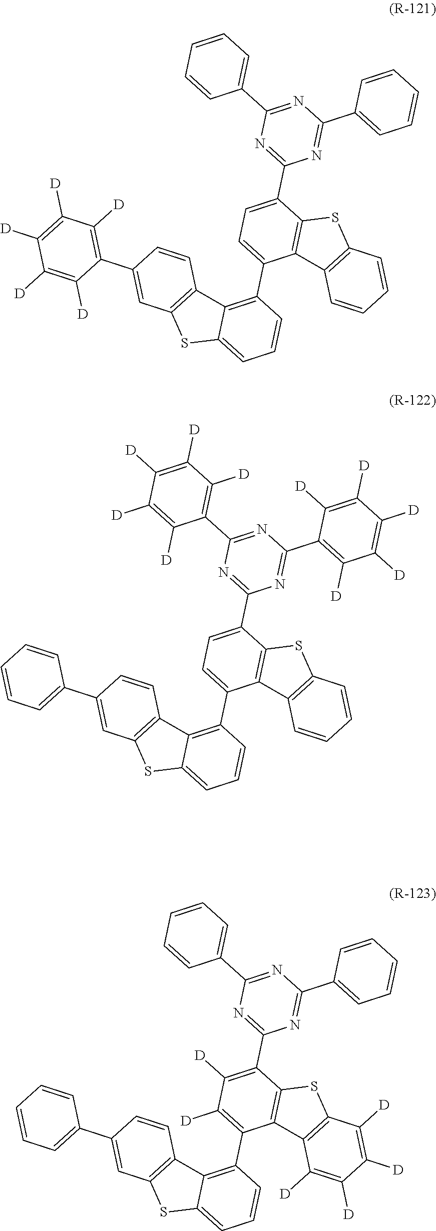 [SMILES: [2H]c1c([2H])c([2H])c(-c2ccc3c(c2)sc2cccc(-c4ccc(-c5nc(-c6ccccc6)nc(-c6ccccc6)n5)c5sc6ccccc6c45)c23)c([2H])c1[2H] ; [2H]c1c([2H])c([2H])c(-c2nc(-c3c([2H])c([2H])c([2H])c([2H])c3[2H])nc(-c3ccc(-c4cccc5sc6cc(-c7ccccc7)ccc6c45)c4c3sc3ccccc34)n2)c([2H])c1[2H] ; [2H]c1c([2H])c([2H])c2c(sc3c(-c4nc(-c5ccccc5)nc(-c5ccccc5)n4)c([2H])c([2H])c(-c4cccc5sc6cc(-c7ccccc7)ccc6c45)c32)c1[2H]]

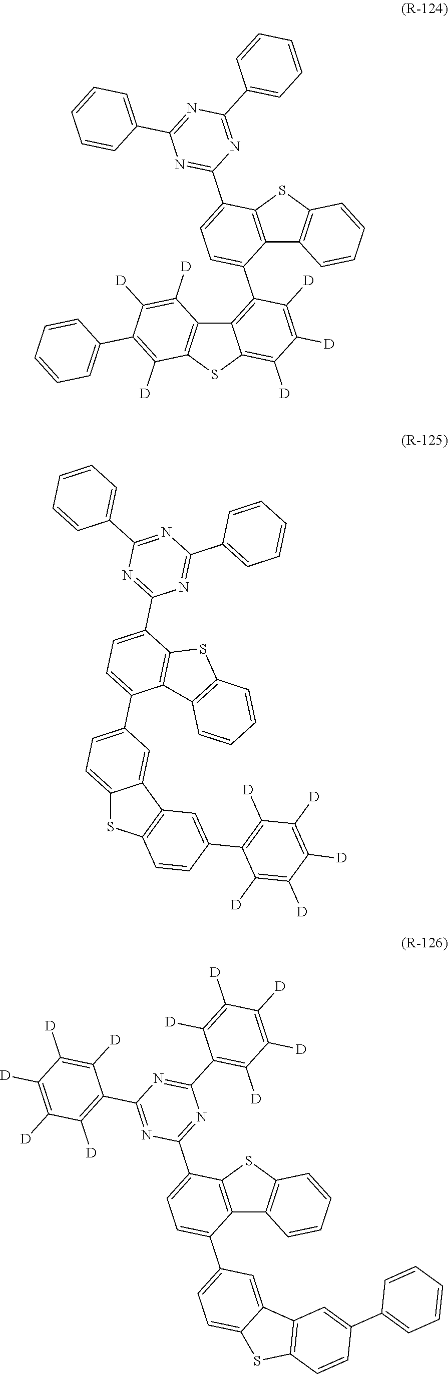 [SMILES: [2H]c1c([2H])c(-c2ccc(-c3nc(-c4ccccc4)nc(-c4ccccc4)n3)c3sc4ccccc4c23)c2c(sc3c([2H])c(-c4ccccc4)c([2H])c([2H])c32)c1[2H] ; [2H]c1c([2H])c([2H])c(-c2ccc3sc4ccc(-c5ccc(-c6nc(-c7ccccc7)nc(-c7ccccc7)n6)c6sc7ccccc7c56)cc4c3c2)c([2H])c1[2H] ; [2H]c1c([2H])c([2H])c(-c2nc(-c3c([2H])c([2H])c([2H])c([2H])c3[2H])nc(-c3ccc(-c4ccc5sc6ccc(-c7ccccc7)cc6c5c4)c4c3sc3ccccc34)n2)c([2H])c1[2H]]

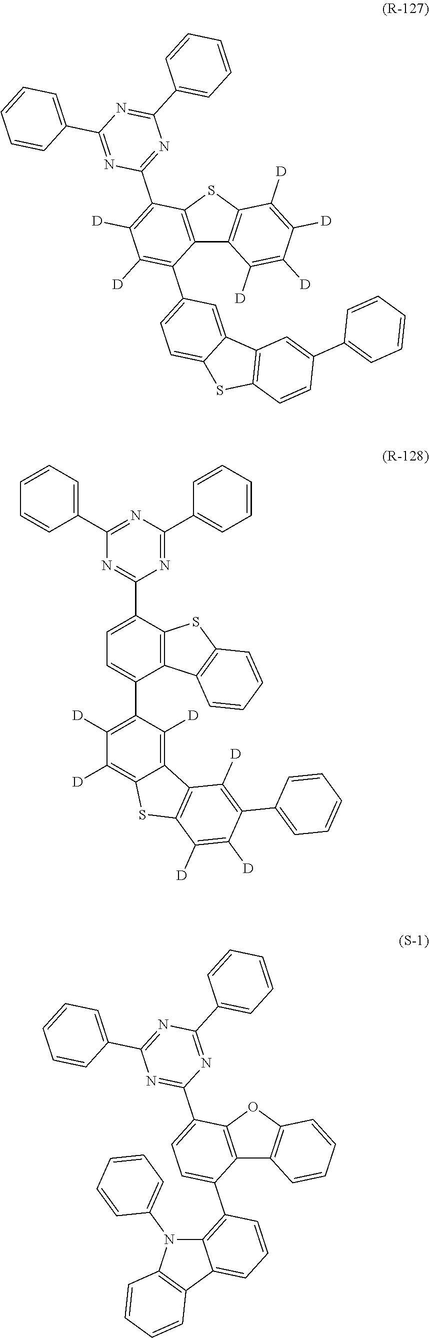 [SMILES: [2H]c1c([2H])c([2H])c2c(sc3c(-c4nc(-c5ccccc5)nc(-c5ccccc5)n4)c([2H])c([2H])c(-c4ccc5sc6ccc(-c7ccccc7)cc6c5c4)c32)c1[2H] ; [2H]c1c(-c2ccccc2)c([2H])c2c(sc3c([2H])c([2H])c(-c4ccc(-c5nc(-c6ccccc6)nc(-c6ccccc6)n5)c5sc6ccccc6c45)c([2H])c32)c1[2H] ; c1ccc(-c2nc(-c3ccccc3)nc(-c3ccc(-c4cccc5c6ccccc6n(-c6ccccc6)c45)c4c3oc3ccccc34)n2)cc1]

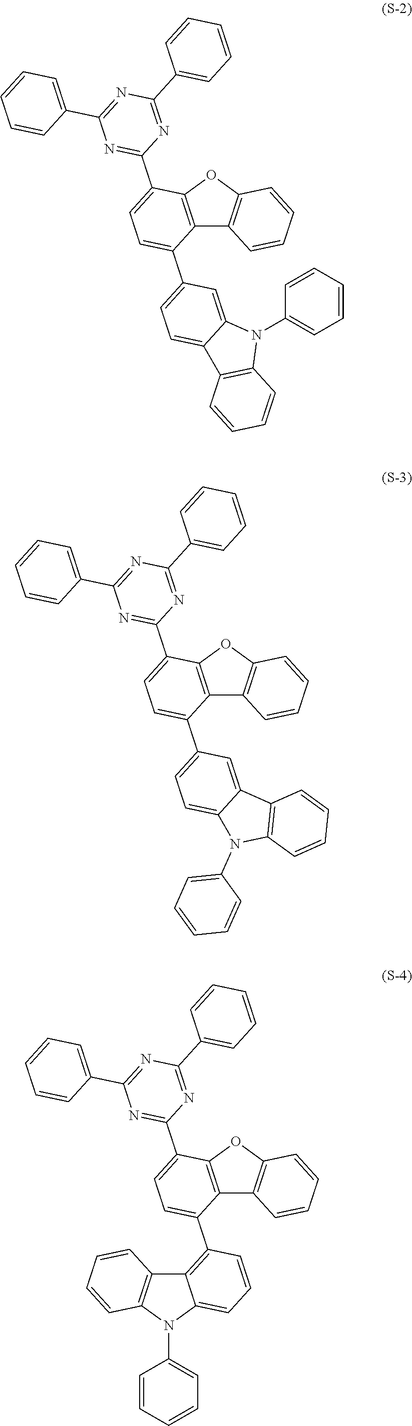 [SMILES: c1ccc(-c2nc(-c3ccccc3)nc(-c3ccc(-c4ccc5c6ccccc6n(-c6ccccc6)c5c4)c4c3oc3ccccc34)n2)cc1 ; c1ccc(-c2nc(-c3ccccc3)nc(-c3ccc(-c4ccc5c(c4)c4ccccc4n5-c4ccccc4)c4c3oc3ccccc34)n2)cc1 ; c1ccc(-c2nc(-c3ccccc3)nc(-c3ccc(-c4cccc5c4c4ccccc4n5-c4ccccc4)c4c3oc3ccccc34)n2)cc1]

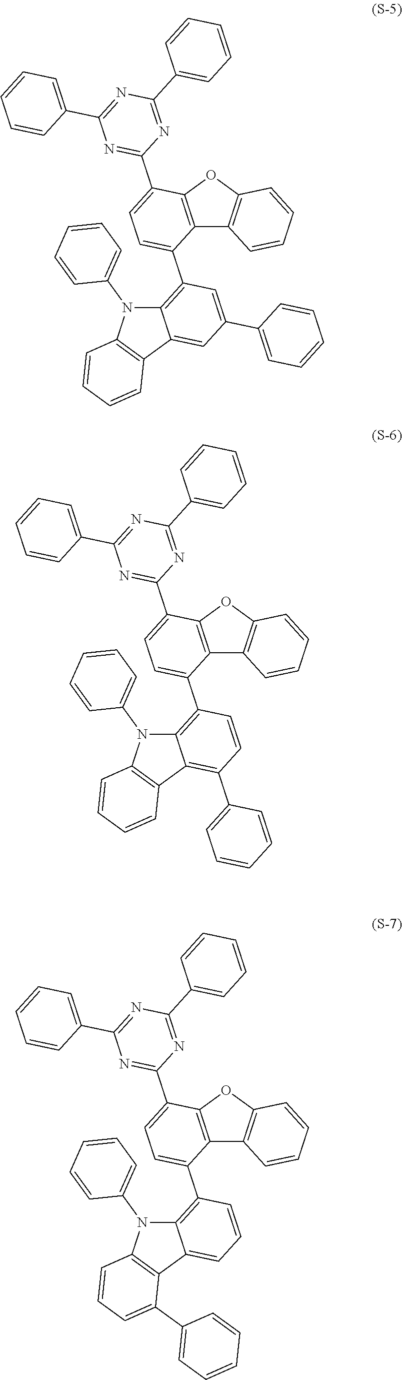 [SMILES: c1ccc(-c2cc(-c3ccc(-c4nc(-c5ccccc5)nc(-c5ccccc5)n4)c4oc5ccccc5c34)c3c(c2)c2ccccc2n3-c2ccccc2)cc1 ; c1ccc(-c2nc(-c3ccccc3)nc(-c3ccc(-c4ccc(-c5ccccc5)c5c6ccccc6n(-c6ccccc6)c45)c4c3oc3ccccc34)n2)cc1 ; c1ccc(-c2nc(-c3ccccc3)nc(-c3ccc(-c4cccc5c6c(-c7ccccc7)cccc6n(-c6ccccc6)c45)c4c3oc3ccccc34)n2)cc1]

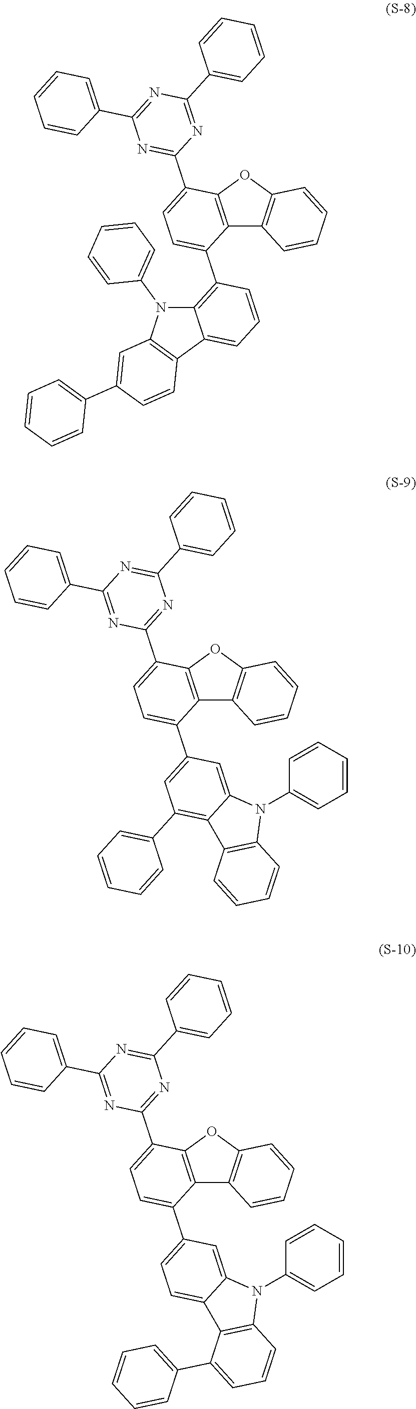 [SMILES: c1ccc(-c2ccc3c4cccc(-c5ccc(-c6nc(-c7ccccc7)nc(-c7ccccc7)n6)c6oc7ccccc7c56)c4n(-c4ccccc4)c3c2)cc1 ; c1ccc(-c2nc(-c3ccccc3)nc(-c3ccc(-c4cc(-c5ccccc5)c5c6ccccc6n(-c6ccccc6)c5c4)c4c3oc3ccccc34)n2)cc1 ; c1ccc(-c2nc(-c3ccccc3)nc(-c3ccc(-c4ccc5c6c(-c7ccccc7)cccc6n(-c6ccccc6)c5c4)c4c3oc3ccccc34)n2)cc1]

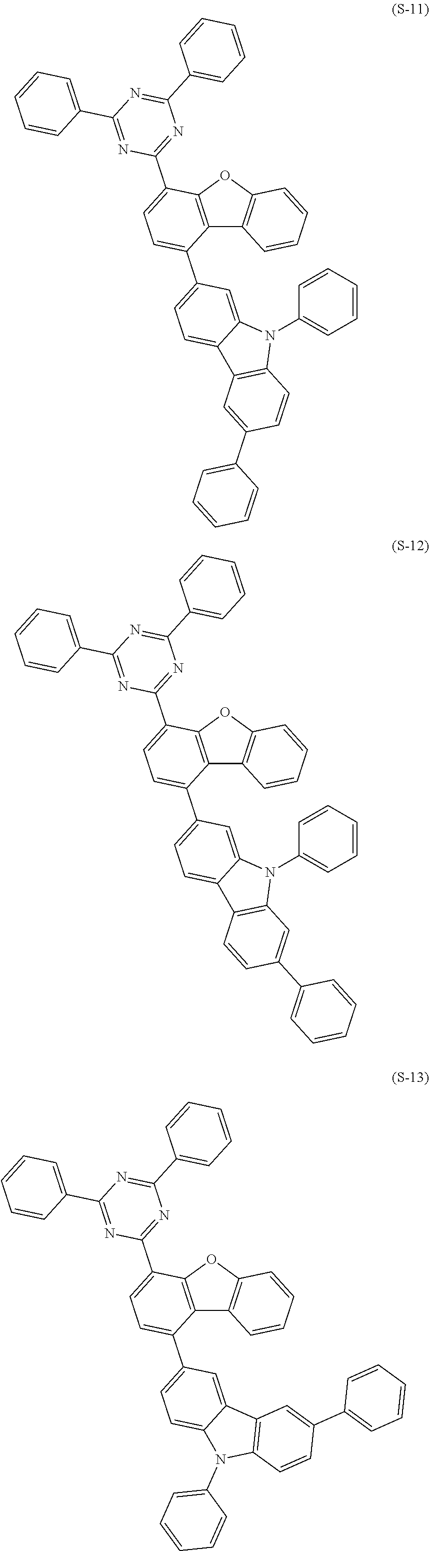

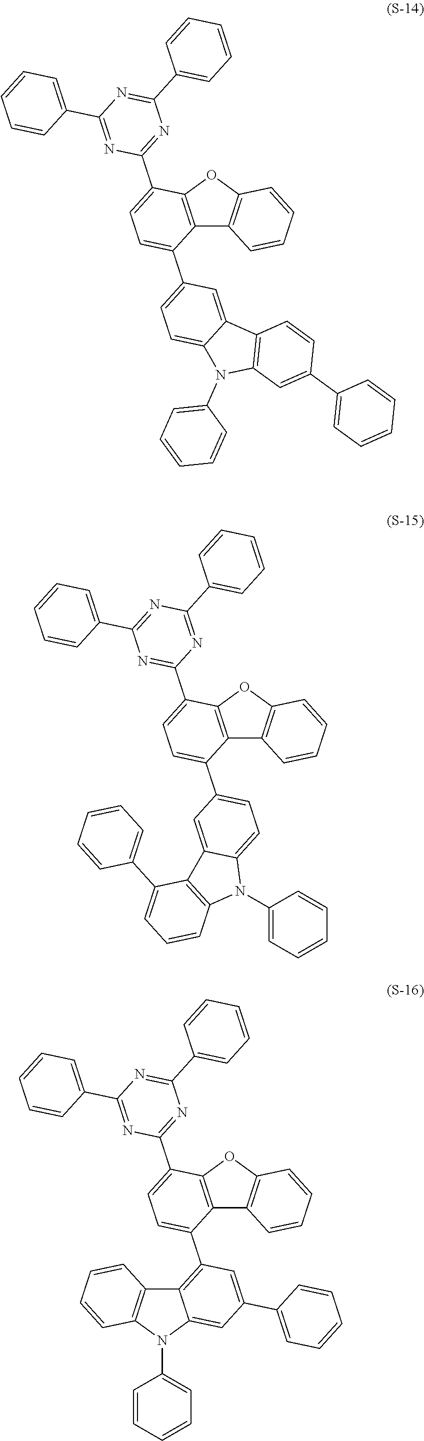 [SMILES: c1ccc(-c2ccc3c4cc(-c5ccc(-c6nc(-c7ccccc7)nc(-c7ccccc7)n6)c6oc7ccccc7c56)ccc4n(-c4ccccc4)c3c2)cc1 ; c1ccc(-c2nc(-c3ccccc3)nc(-c3ccc(-c4ccc5c(c4)c4c(-c6ccccc6)cccc4n5-c4ccccc4)c4c3oc3ccccc34)n2)cc1 ; c1ccc(-c2cc(-c3ccc(-c4nc(-c5ccccc5)nc(-c5ccccc5)n4)c4oc5ccccc5c34)c3c4ccccc4n(-c4ccccc4)c3c2)cc1]

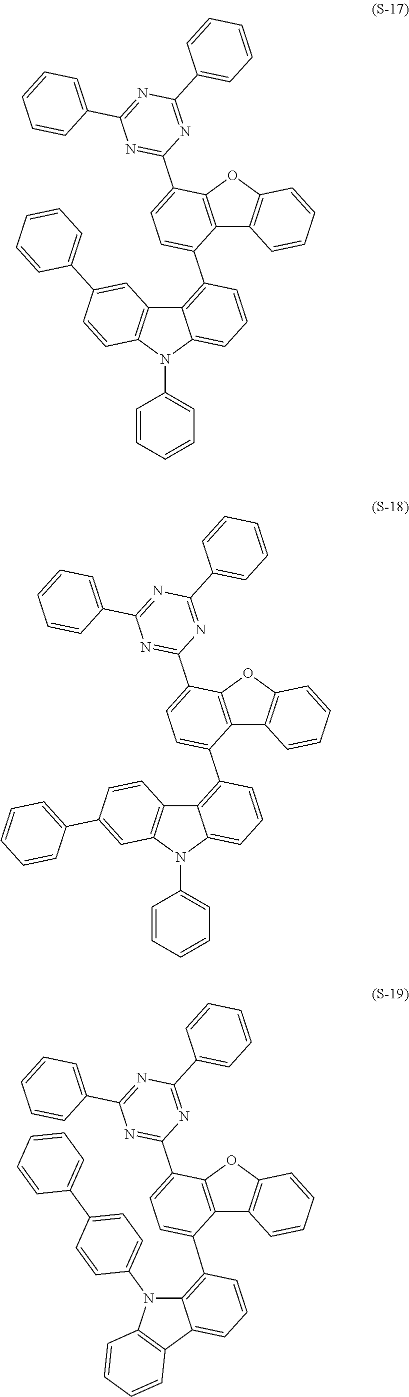 [SMILES: c1ccc(-c2ccc3c(c2)c2c(-c4ccc(-c5nc(-c6ccccc6)nc(-c6ccccc6)n5)c5oc6ccccc6c45)cccc2n3-c2ccccc2)cc1 ; c1ccc(-c2ccc3c4c(-c5ccc(-c6nc(-c7ccccc7)nc(-c7ccccc7)n6)c6oc7ccccc7c56)cccc4n(-c4ccccc4)c3c2)cc1 ; c1ccc(-c2ccc(-n3c4ccccc4c4cccc(-c5ccc(-c6nc(-c7ccccc7)nc(-c7ccccc7)n6)c6oc7ccccc7c56)c43)cc2)cc1]

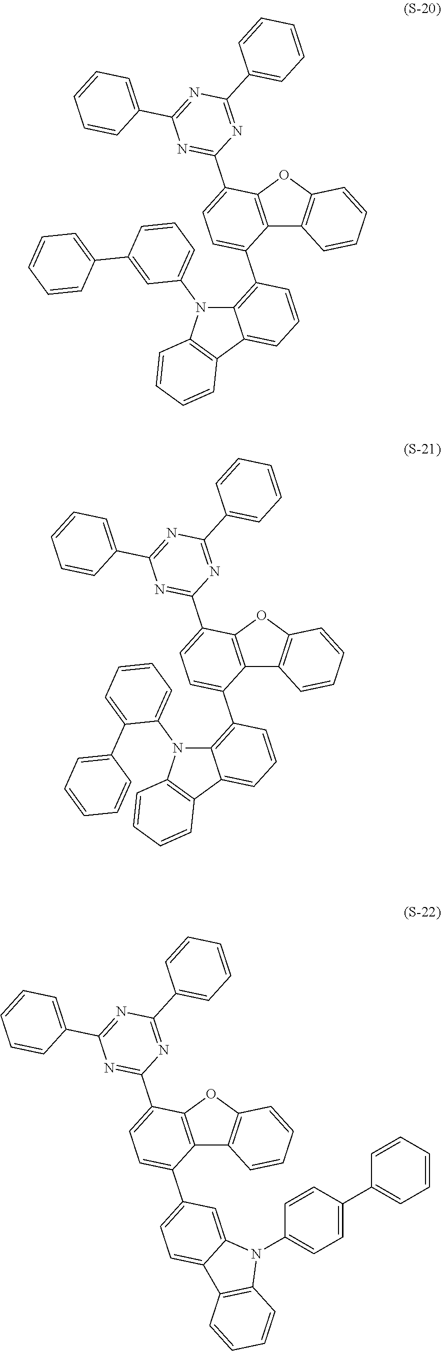 [SMILES: c1ccc(-c2cccc(-n3c4ccccc4c4cccc(-c5ccc(-c6nc(-c7ccccc7)nc(-c7ccccc7)n6)c6oc7ccccc7c56)c43)c2)cc1 ; c1ccc(-c2nc(-c3ccccc3)nc(-c3ccc(-c4cccc5c6ccccc6n(-c6ccccc6-c6ccccc6)c45)c4c3oc3ccccc34)n2)cc1 ; c1ccc(-c2ccc(-n3c4ccccc4c4ccc(-c5ccc(-c6nc(-c7ccccc7)nc(-c7ccccc7)n6)c6oc7ccccc7c56)cc43)cc2)cc1]

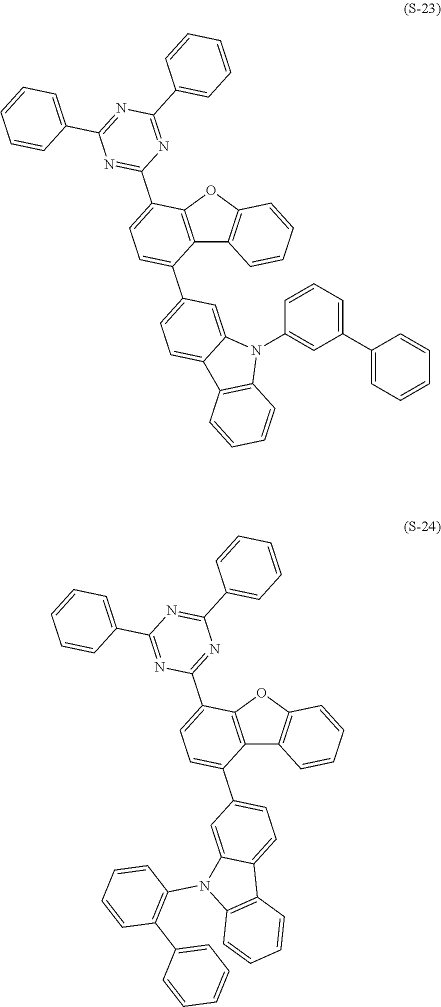 [SMILES: c1ccc(-c2cccc(-n3c4ccccc4c4ccc(-c5ccc(-c6nc(-c7ccccc7)nc(-c7ccccc7)n6)c6oc7ccccc7c56)cc43)c2)cc1 ; c1ccc(-c2nc(-c3ccccc3)nc(-c3ccc(-c4ccc5c6ccccc6n(-c6ccccc6-c6ccccc6)c5c4)c4c3oc3ccccc34)n2)cc1]

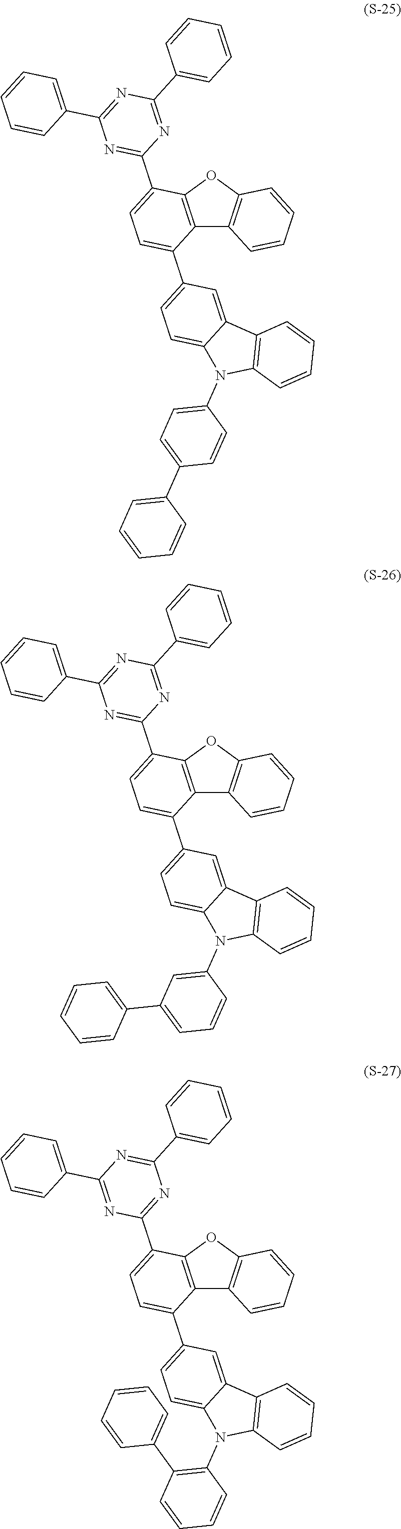

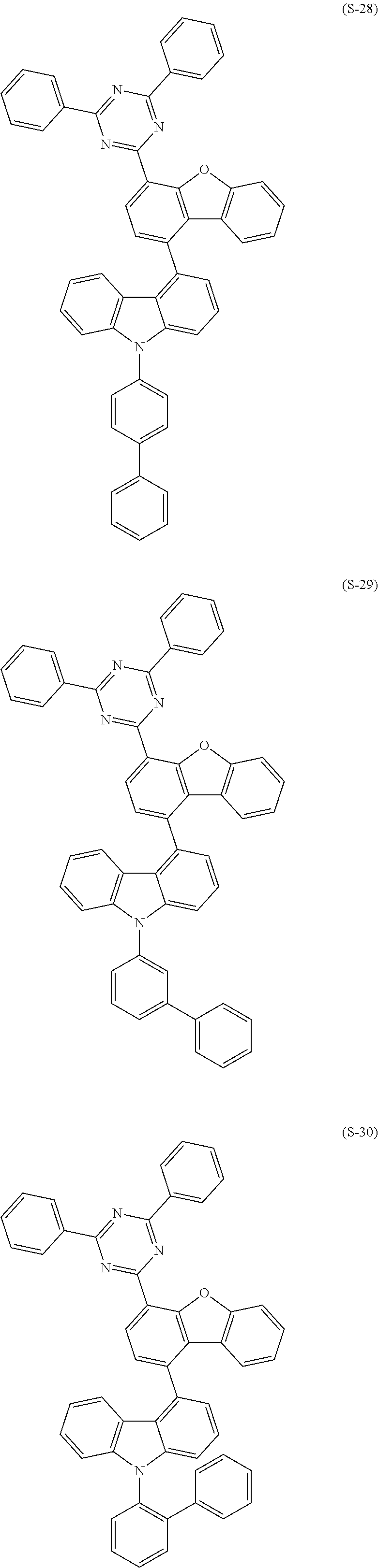 [SMILES: c1ccc(-c2ccc(-n3c4ccccc4c4c(-c5ccc(-c6nc(-c7ccccc7)nc(-c7ccccc7)n6)c6oc7ccccc7c56)cccc43)cc2)cc1 ; c1ccc(-c2cccc(-n3c4ccccc4c4c(-c5ccc(-c6nc(-c7ccccc7)nc(-c7ccccc7)n6)c6oc7ccccc7c56)cccc43)c2)cc1 ; c1ccc(-c2nc(-c3ccccc3)nc(-c3ccc(-c4cccc5c4c4ccccc4n5-c4ccccc4-c4ccccc4)c4c3oc3ccccc34)n2)cc1]

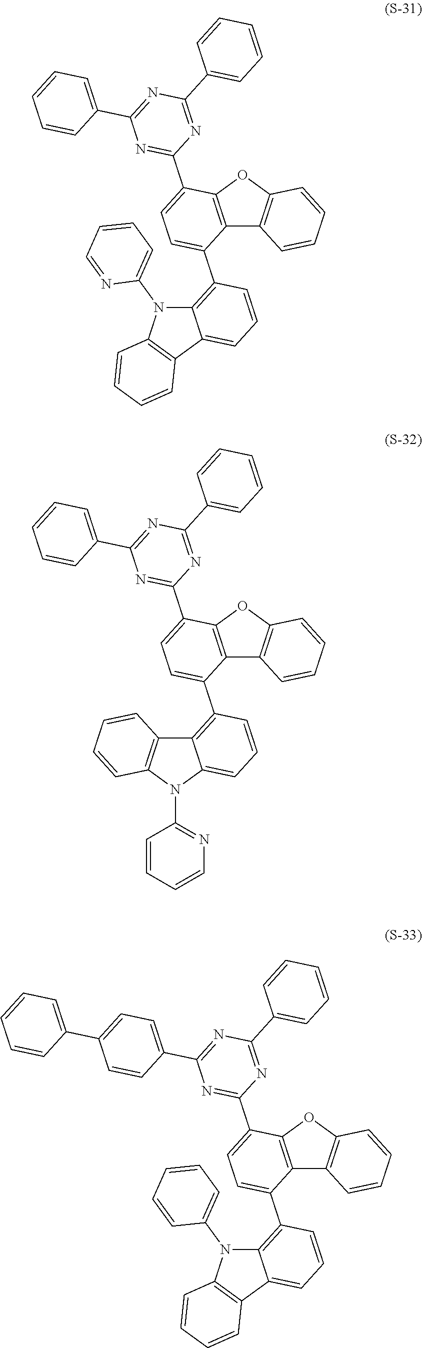 [SMILES: c1ccc(-c2nc(-c3ccccc3)nc(-c3ccc(-c4cccc5c6ccccc6n(-c6ccccn6)c45)c4c3oc3ccccc34)n2)cc1 ; c1ccc(-c2nc(-c3ccccc3)nc(-c3ccc(-c4cccc5c4c4ccccc4n5-c4ccccn4)c4c3oc3ccccc34)n2)cc1 ; c1ccc(-c2ccc(-c3nc(-c4ccccc4)nc(-c4ccc(-c5cccc6c7ccccc7n(-c7ccccc7)c56)c5c4oc4ccccc45)n3)cc2)cc1]

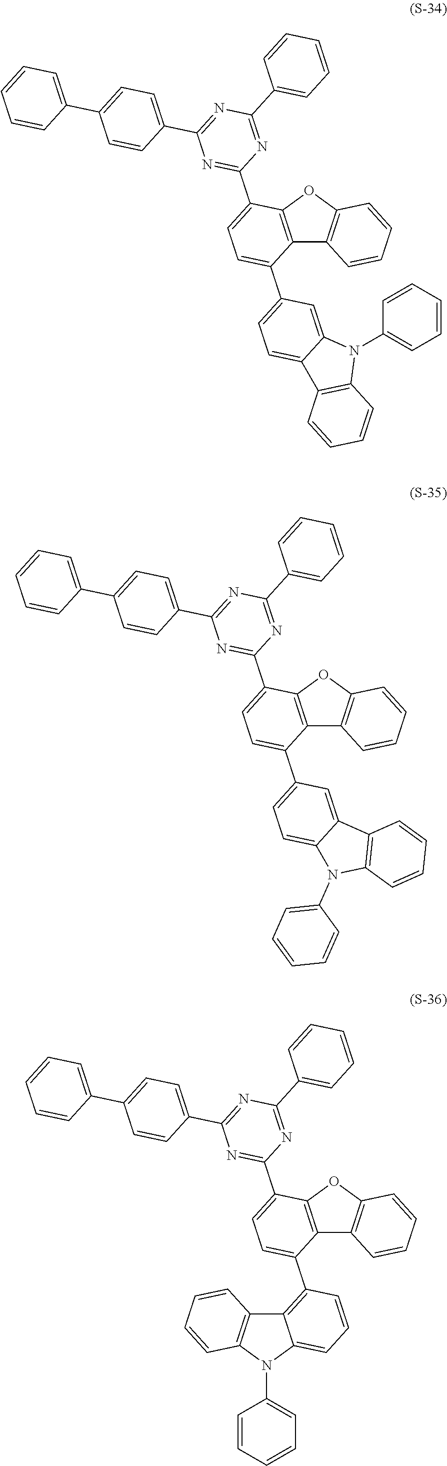 [SMILES: c1ccc(-c2ccc(-c3nc(-c4ccccc4)nc(-c4ccc(-c5ccc6c7ccccc7n(-c7ccccc7)c6c5)c5c4oc4ccccc45)n3)cc2)cc1 ; c1ccc(-c2ccc(-c3nc(-c4ccccc4)nc(-c4ccc(-c5ccc6c(c5)c5ccccc5n6-c5ccccc5)c5c4oc4ccccc45)n3)cc2)cc1 ; c1ccc(-c2ccc(-c3nc(-c4ccccc4)nc(-c4ccc(-c5cccc6c5c5ccccc5n6-c5ccccc5)c5c4oc4ccccc45)n3)cc2)cc1]

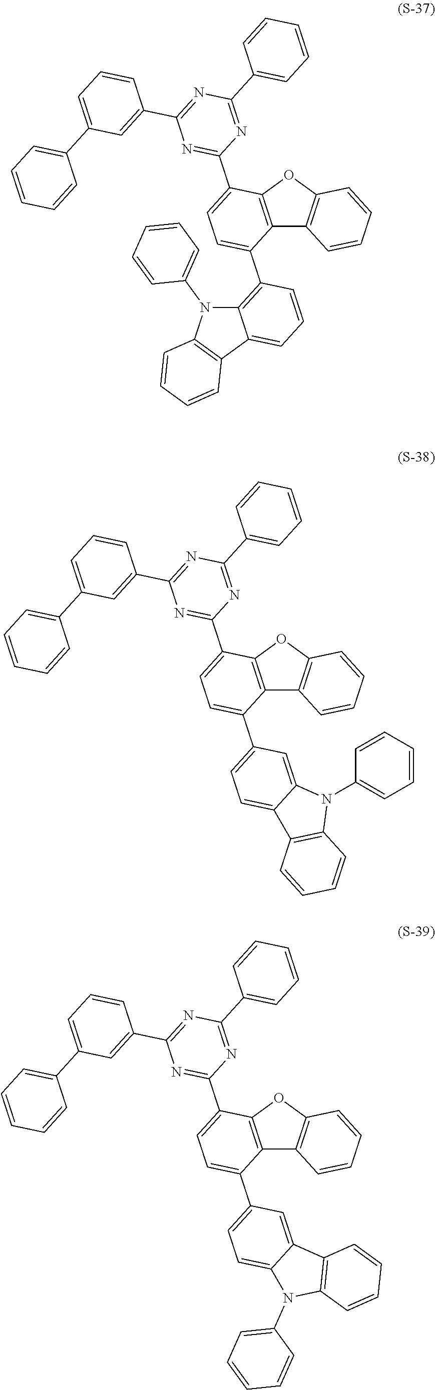 [SMILES: c1ccc(-c2cccc(-c3nc(-c4ccccc4)nc(-c4ccc(-c5cccc6c7ccccc7n(-c7ccccc7)c56)c5c4oc4ccccc45)n3)c2)cc1 ; c1ccc(-c2cccc(-c3nc(-c4ccccc4)nc(-c4ccc(-c5ccc6c7ccccc7n(-c7ccccc7)c6c5)c5c4oc4ccccc45)n3)c2)cc1 ; c1ccc(-c2cccc(-c3nc(-c4ccccc4)nc(-c4ccc(-c5ccc6c(c5)c5ccccc5n6-c5ccccc5)c5c4oc4ccccc45)n3)c2)cc1]

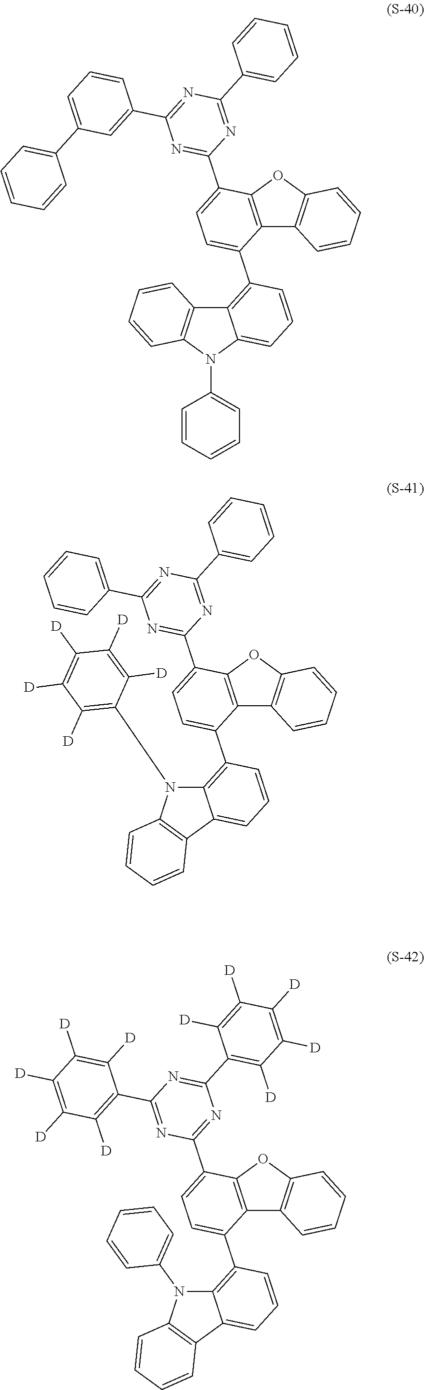

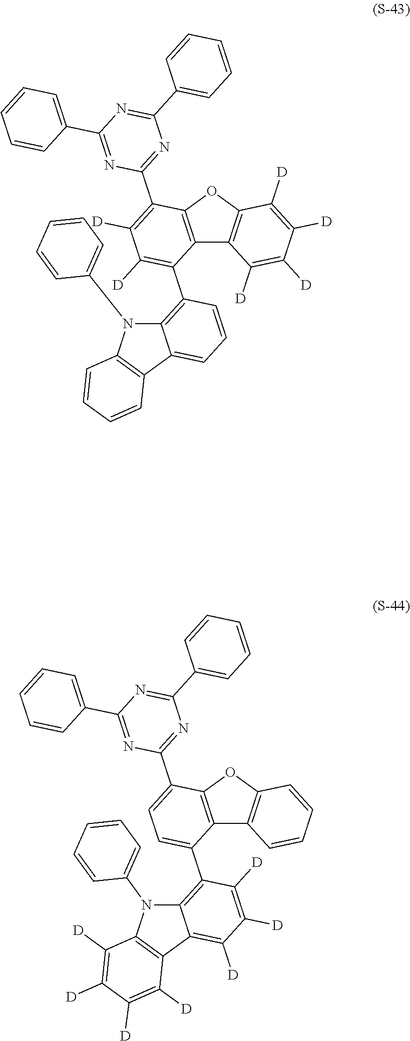 [SMILES: [2H]c1c([2H])c([2H])c2c(oc3c(-c4nc(-c5ccccc5)nc(-c5ccccc5)n4)c([2H])c([2H])c(-c4cccc5c6ccccc6n(-c6ccccc6)c45)c32)c1[2H] ; [2H]c1c([2H])c([2H])c2c(c1[2H])c1c([2H])c([2H])c([2H])c(-c3ccc(-c4nc(-c5ccccc5)nc(-c5ccccc5)n4)c4oc5ccccc5c34)c1n2-c1ccccc1]

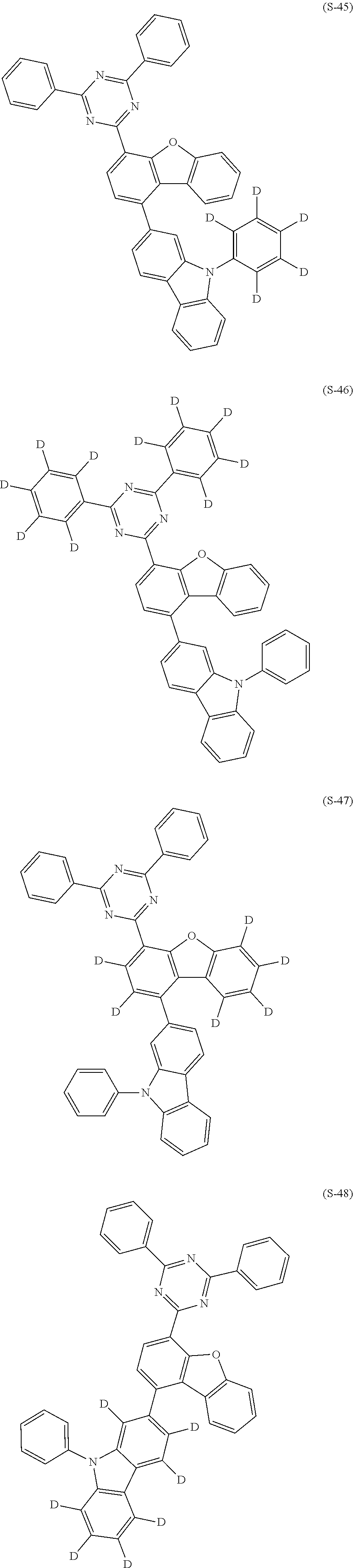

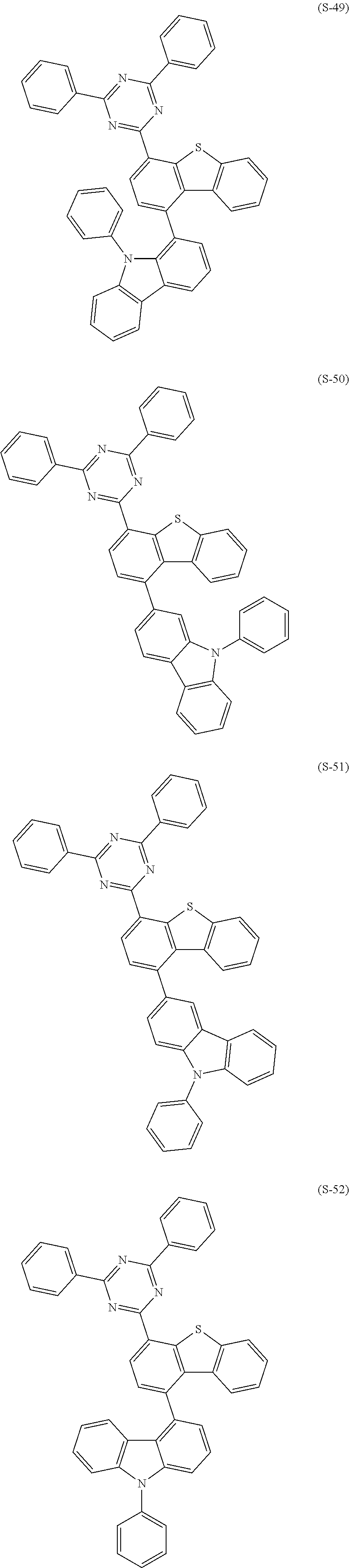 [SMILES: c1ccc(-c2nc(-c3ccccc3)nc(-c3ccc(-c4cccc5c6ccccc6n(-c6ccccc6)c45)c4c3sc3ccccc34)n2)cc1 ; c1ccc(-c2nc(-c3ccccc3)nc(-c3ccc(-c4ccc5c6ccccc6n(-c6ccccc6)c5c4)c4c3sc3ccccc34)n2)cc1 ; c1ccc(-c2nc(-c3ccccc3)nc(-c3ccc(-c4ccc5c(c4)c4ccccc4n5-c4ccccc4)c4c3sc3ccccc34)n2)cc1 ; c1ccc(-c2nc(-c3ccccc3)nc(-c3ccc(-c4cccc5c4c4ccccc4n5-c4ccccc4)c4c3sc3ccccc34)n2)cc1]

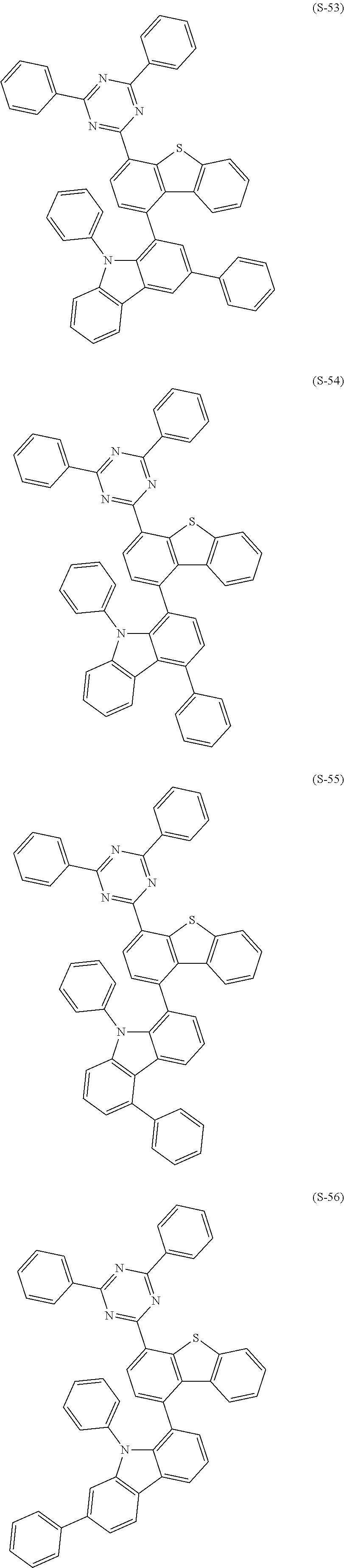 [SMILES: c1ccc(-c2cc(-c3ccc(-c4nc(-c5ccccc5)nc(-c5ccccc5)n4)c4sc5ccccc5c34)c3c(c2)c2ccccc2n3-c2ccccc2)cc1 ; c1ccc(-c2nc(-c3ccccc3)nc(-c3ccc(-c4ccc(-c5ccccc5)c5c6ccccc6n(-c6ccccc6)c45)c4c3sc3ccccc34)n2)cc1 ; c1ccc(-c2nc(-c3ccccc3)nc(-c3ccc(-c4cccc5c6c(-c7ccccc7)cccc6n(-c6ccccc6)c45)c4c3sc3ccccc34)n2)cc1 ; c1ccc(-c2ccc3c4cccc(-c5ccc(-c6nc(-c7ccccc7)nc(-c7ccccc7)n6)c6sc7ccccc7c56)c4n(-c4ccccc4)c3c2)cc1]

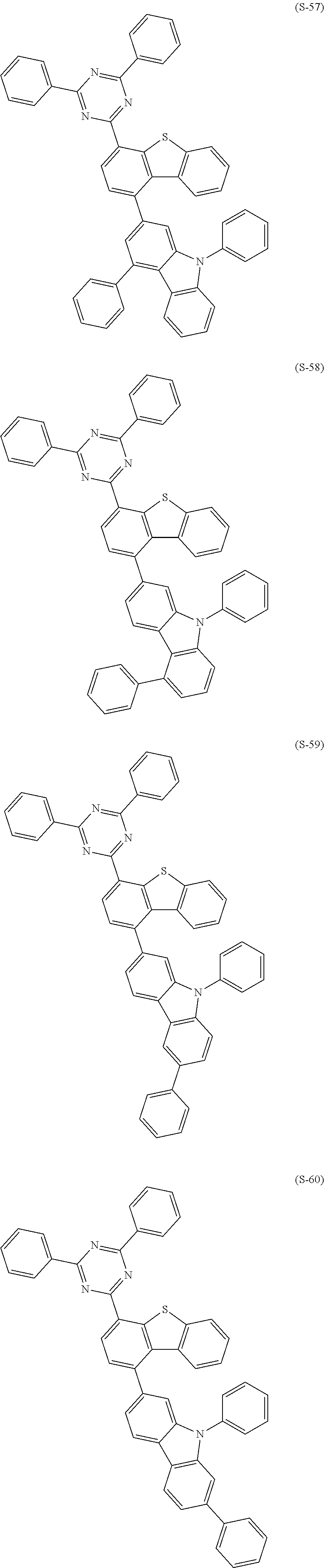 [SMILES: c1ccc(-c2nc(-c3ccccc3)nc(-c3ccc(-c4cc(-c5ccccc5)c5c6ccccc6n(-c6ccccc6)c5c4)c4c3sc3ccccc34)n2)cc1 ; c1ccc(-c2nc(-c3ccccc3)nc(-c3ccc(-c4ccc5c6c(-c7ccccc7)cccc6n(-c6ccccc6)c5c4)c4c3sc3ccccc34)n2)cc1 ; c1ccc(-c2ccc3c(c2)c2ccc(-c4ccc(-c5nc(-c6ccccc6)nc(-c6ccccc6)n5)c5sc6ccccc6c45)cc2n3-c2ccccc2)cc1 ; c1ccc(-c2ccc3c4ccc(-c5ccc(-c6nc(-c7ccccc7)nc(-c7ccccc7)n6)c6sc7ccccc7c56)cc4n(-c4ccccc4)c3c2)cc1]

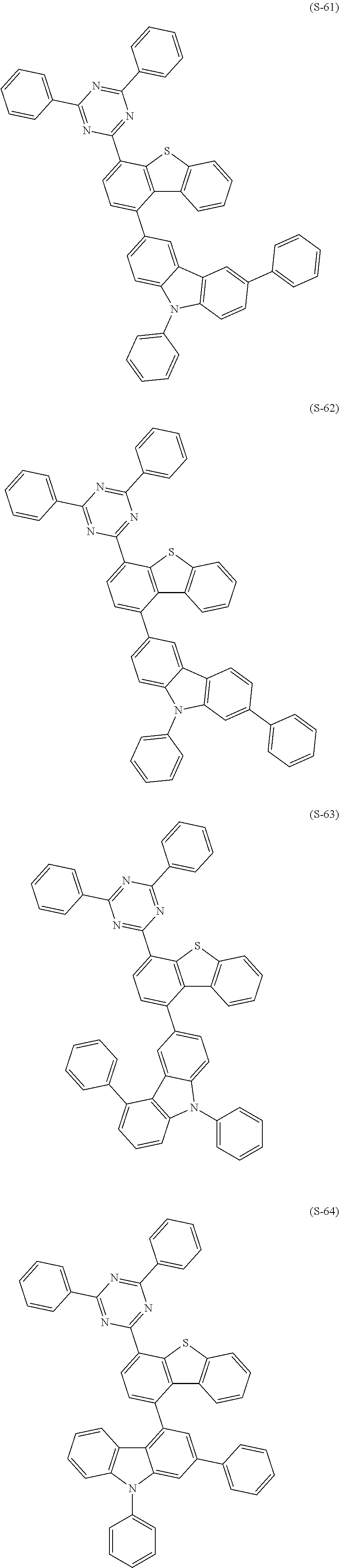 [SMILES: c1ccc(-c2ccc3c(c2)c2cc(-c4ccc(-c5nc(-c6ccccc6)nc(-c6ccccc6)n5)c5sc6ccccc6c45)ccc2n3-c2ccccc2)cc1 ; c1ccc(-c2ccc3c4cc(-c5ccc(-c6nc(-c7ccccc7)nc(-c7ccccc7)n6)c6sc7ccccc7c56)ccc4n(-c4ccccc4)c3c2)cc1 ; c1ccc(-c2nc(-c3ccccc3)nc(-c3ccc(-c4ccc5c(c4)c4c(-c6ccccc6)cccc4n5-c4ccccc4)c4c3sc3ccccc34)n2)cc1 ; c1ccc(-c2cc(-c3ccc(-c4nc(-c5ccccc5)nc(-c5ccccc5)n4)c4sc5ccccc5c34)c3c4ccccc4n(-c4ccccc4)c3c2)cc1]

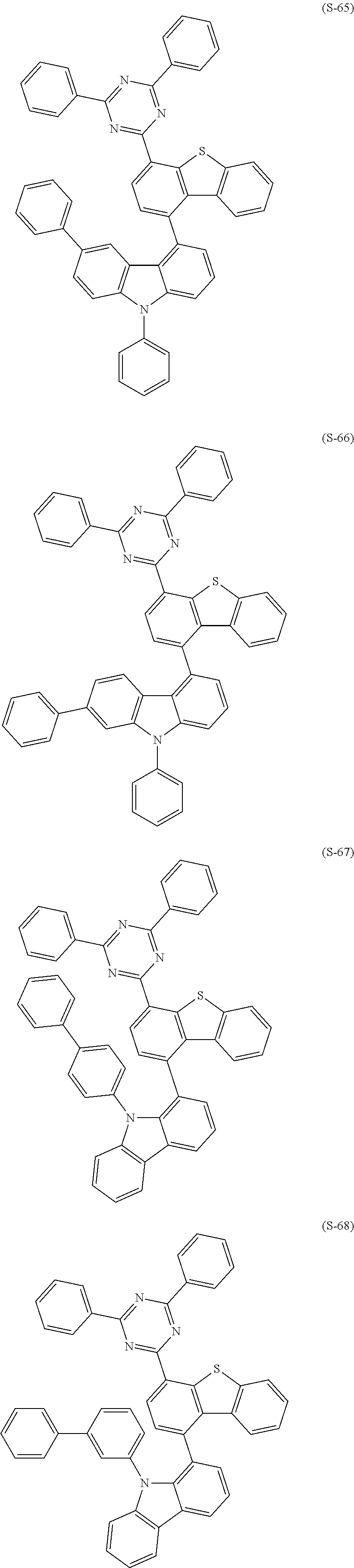 [SMILES: c1ccc(-c2ccc3c(c2)c2c(-c4ccc(-c5nc(-c6ccccc6)nc(-c6ccccc6)n5)c5sc6ccccc6c45)cccc2n3-c2ccccc2)cc1 ; c1ccc(-c2ccc3c4c(-c5ccc(-c6nc(-c7ccccc7)nc(-c7ccccc7)n6)c6sc7ccccc7c56)cccc4n(-c4ccccc4)c3c2)cc1 ; c1ccc(-c2ccc(-n3c4ccccc4c4cccc(-c5ccc(-c6nc(-c7ccccc7)nc(-c7ccccc7)n6)c6sc7ccccc7c56)c43)cc2)cc1 ; c1ccc(-c2cccc(-n3c4ccccc4c4cccc(-c5ccc(-c6nc(-c7ccccc7)nc(-c7ccccc7)n6)c6sc7ccccc7c56)c43)c2)cc1]

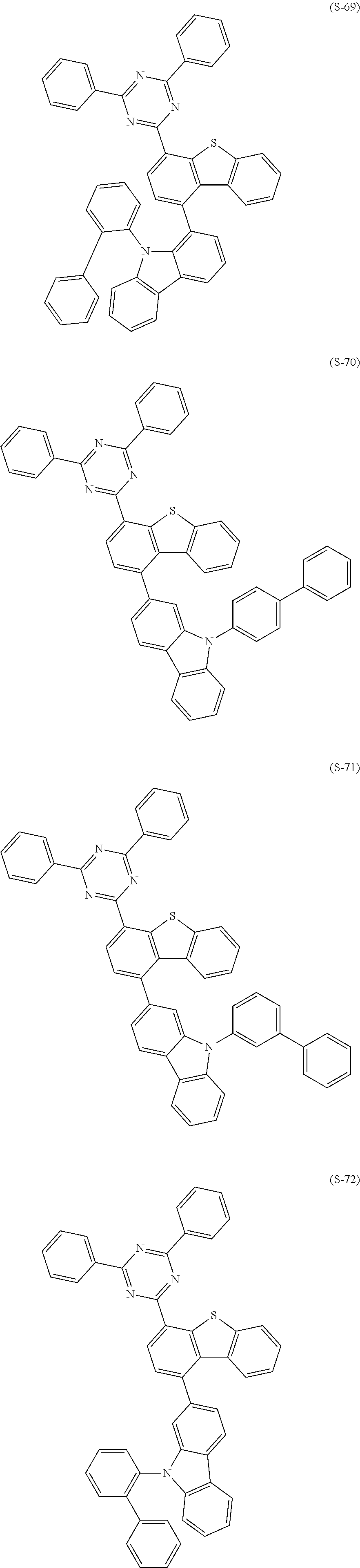 [SMILES: c1ccc(-c2nc(-c3ccccc3)nc(-c3ccc(-c4cccc5c6ccccc6n(-c6ccccc6-c6ccccc6)c45)c4c3sc3ccccc34)n2)cc1 ; c1ccc(-c2ccc(-n3c4ccccc4c4ccc(-c5ccc(-c6nc(-c7ccccc7)nc(-c7ccccc7)n6)c6sc7ccccc7c56)cc43)cc2)cc1 ; c1ccc(-c2cccc(-n3c4ccccc4c4ccc(-c5ccc(-c6nc(-c7ccccc7)nc(-c7ccccc7)n6)c6sc7ccccc7c56)cc43)c2)cc1 ; c1ccc(-c2nc(-c3ccccc3)nc(-c3ccc(-c4ccc5c6ccccc6n(-c6ccccc6-c6ccccc6)c5c4)c4c3sc3ccccc34)n2)cc1]

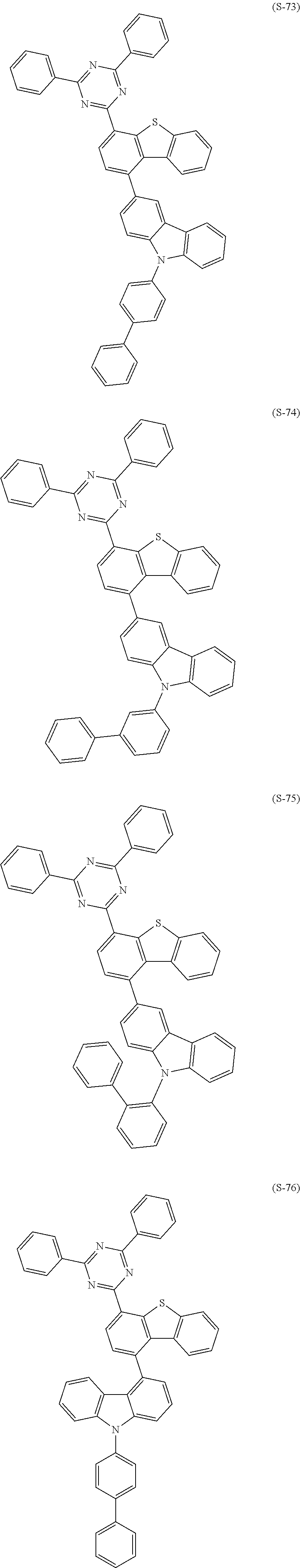 [SMILES: c1ccc(-c2ccc(-n3c4ccccc4c4cc(-c5ccc(-c6nc(-c7ccccc7)nc(-c7ccccc7)n6)c6sc7ccccc7c56)ccc43)cc2)cc1 ; c1ccc(-c2cccc(-n3c4ccccc4c4cc(-c5ccc(-c6nc(-c7ccccc7)nc(-c7ccccc7)n6)c6sc7ccccc7c56)ccc43)c2)cc1 ; c1ccc(-c2nc(-c3ccccc3)nc(-c3ccc(-c4ccc5c(c4)c4ccccc4n5-c4ccccc4-c4ccccc4)c4c3sc3ccccc34)n2)cc1 ; c1ccc(-c2ccc(-n3c4ccccc4c4c(-c5ccc(-c6nc(-c7ccccc7)nc(-c7ccccc7)n6)c6sc7ccccc7c56)cccc43)cc2)cc1]

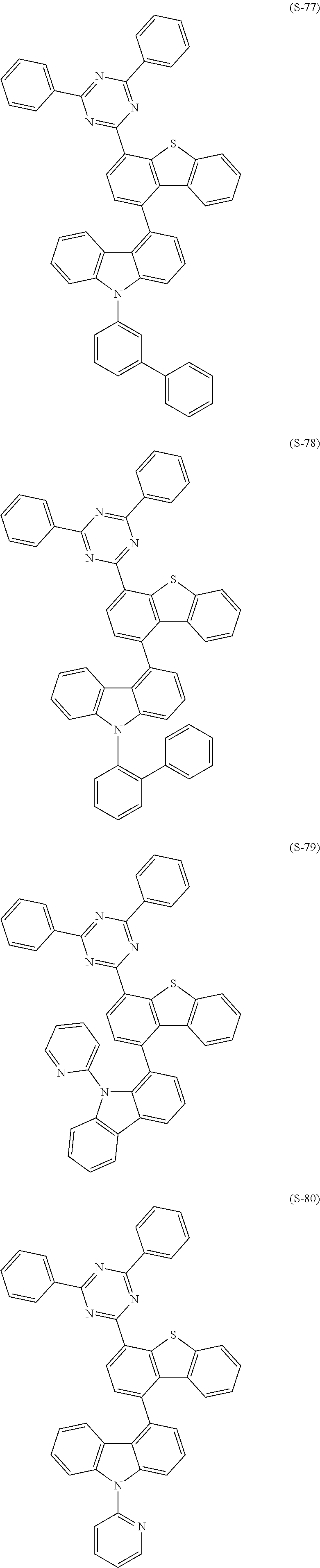 [SMILES: c1ccc(-c2cccc(-n3c4ccccc4c4c(-c5ccc(-c6nc(-c7ccccc7)nc(-c7ccccc7)n6)c6sc7ccccc7c56)cccc43)c2)cc1 ; c1ccc(-c2nc(-c3ccccc3)nc(-c3ccc(-c4cccc5c4c4ccccc4n5-c4ccccc4-c4ccccc4)c4c3sc3ccccc34)n2)cc1 ; c1ccc(-c2nc(-c3ccccc3)nc(-c3ccc(-c4cccc5c6ccccc6n(-c6ccccn6)c45)c4c3sc3ccccc34)n2)cc1 ; c1ccc(-c2nc(-c3ccccc3)nc(-c3ccc(-c4cccc5c4c4ccccc4n5-c4ccccn4)c4c3sc3ccccc34)n2)cc1]

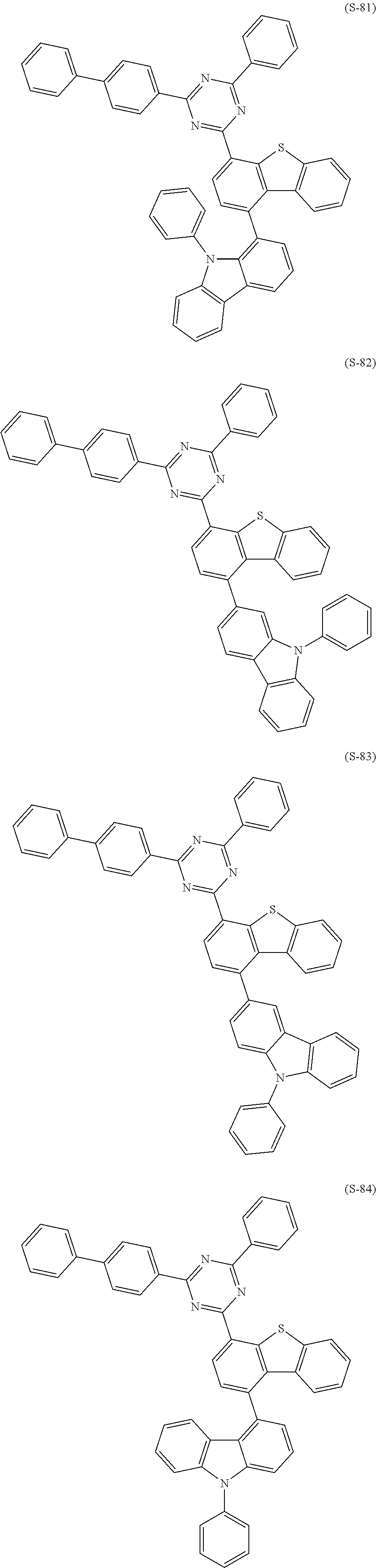 [SMILES: c1ccc(-c2ccc(-c3nc(-c4ccccc4)nc(-c4ccc(-c5cccc6c7ccccc7n(-c7ccccc7)c56)c5c4sc4ccccc45)n3)cc2)cc1 ; c1ccc(-c2ccc(-c3nc(-c4ccccc4)nc(-c4ccc(-c5ccc6c7ccccc7n(-c7ccccc7)c6c5)c5c4sc4ccccc45)n3)cc2)cc1 ; c1ccc(-c2ccc(-c3nc(-c4ccccc4)nc(-c4ccc(-c5ccc6c(c5)c5ccccc5n6-c5ccccc5)c5c4sc4ccccc45)n3)cc2)cc1 ; c1ccc(-c2ccc(-c3nc(-c4ccccc4)nc(-c4ccc(-c5cccc6c5c5ccccc5n6-c5ccccc5)c5c4sc4ccccc45)n3)cc2)cc1]

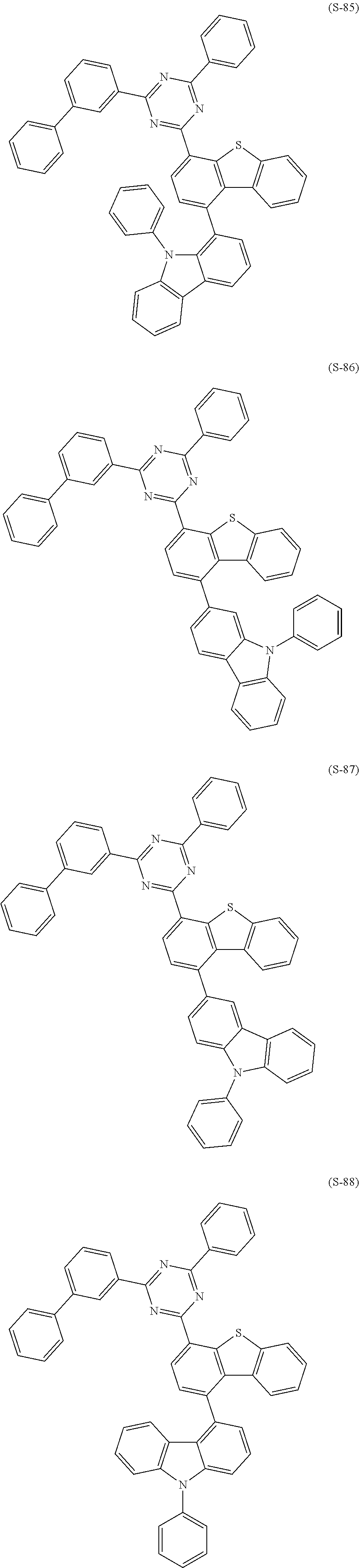 [SMILES: c1ccc(-c2cccc(-c3nc(-c4ccccc4)nc(-c4ccc(-c5cccc6c7ccccc7n(-c7ccccc7)c56)c5c4sc4ccccc45)n3)c2)cc1 ; c1ccc(-c2cccc(-c3nc(-c4ccccc4)nc(-c4ccc(-c5ccc6c7ccccc7n(-c7ccccc7)c6c5)c5c4sc4ccccc45)n3)c2)cc1 ; c1ccc(-c2cccc(-c3nc(-c4ccccc4)nc(-c4ccc(-c5ccc6c(c5)c5ccccc5n6-c5ccccc5)c5c4sc4ccccc45)n3)c2)cc1 ; c1ccc(-c2cccc(-c3nc(-c4ccccc4)nc(-c4ccc(-c5cccc6c5c5ccccc5n6-c5ccccc5)c5c4sc4ccccc45)n3)c2)cc1]

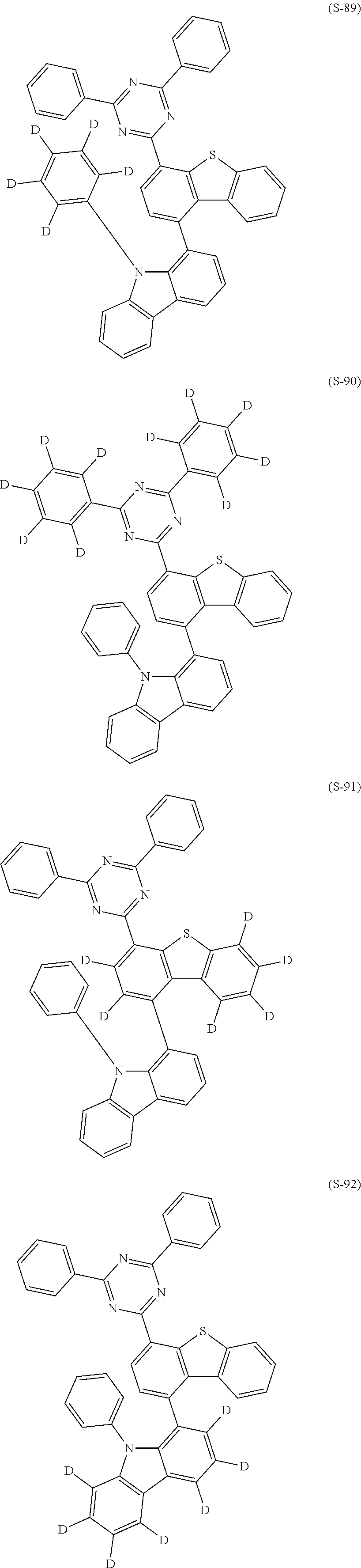

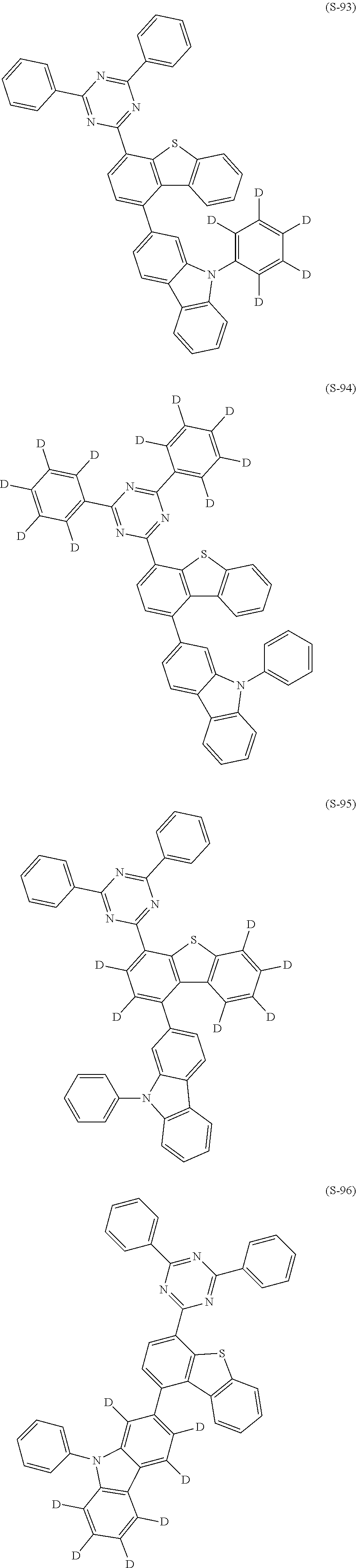

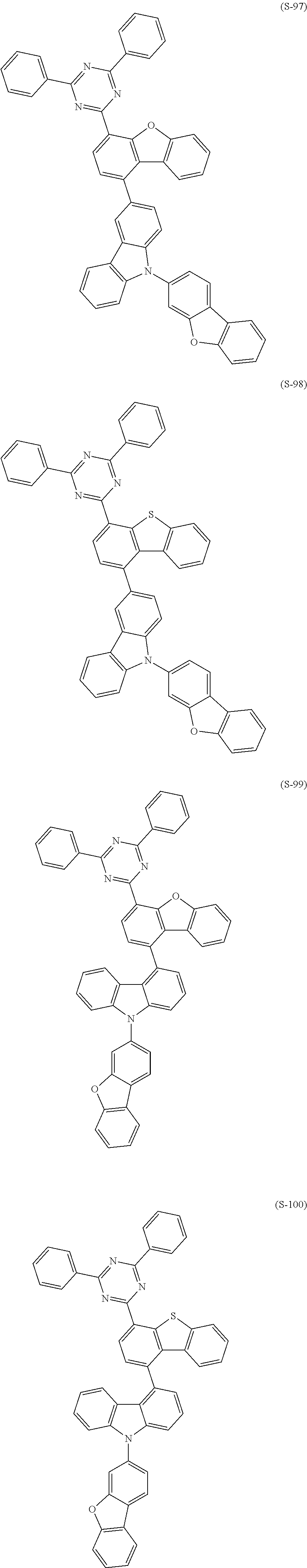 [SMILES: c1ccc(-c2nc(-c3ccccc3)nc(-c3ccc(-c4ccc5c(c4)c4ccccc4n5-c4ccc5c(c4)oc4ccccc45)c4c3oc3ccccc34)n2)cc1 ; c1ccc(-c2nc(-c3ccccc3)nc(-c3ccc(-c4ccc5c(c4)c4ccccc4n5-c4ccc5c(c4)oc4ccccc45)c4c3sc3ccccc34)n2)cc1 ; c1ccc(-c2nc(-c3ccccc3)nc(-c3ccc(-c4cccc5c4c4ccccc4n5-c4ccc5c(c4)oc4ccccc45)c4c3oc3ccccc34)n2)cc1 ; c1ccc(-c2nc(-c3ccccc3)nc(-c3ccc(-c4cccc5c4c4ccccc4n5-c4ccc5c(c4)oc4ccccc45)c4c3sc3ccccc34)n2)cc1]

In some embodiments, the organic compound is used as a functional material of an organic functional layer of an organic electronic device. The organic functional layer is selected from at least one of a hole injection layer, a hole transport layer, an electron transport layer, an electron injection layer, an electron blocking layer, a hole blocking layer, a light-emitting auxiliary layer (prime layer), and a light-emitting layer.

In some embodiments, the organic compound is used in a light-emitting layer. For example, the organic compound is used as a host material of the light-emitting layer.

Some embodiments of the disclosure provide a mixture, which includes at least one of the organic compounds as described above and at least one organic functional material. The organic functional material is selected from at least one of a hole injection material, a hole transport material, an electron injection material, an electron transport material, a light-emitting auxiliary material, a hole blocking material, a guest material, a host material, and quantum dots.

Some embodiments of the disclosure provide a composition, the composition includes at least one organic solvent and at least one of the organic compounds as described above, or, the composition includes at least one organic solvent and the mixture as described above.

The at least one organic solvent includes a first organic solvent, and the first organic solvent is selected from at least one of an aromatic-based solvent, an heteroaromatic-based solvent, an ester-based solvent, an aromatic ketone-based solvent, an aromatic ether-based solvent, an aliphatic ketone-based solvent, an aliphatic ether-based solvent, an alicyclic compound, an olefin compound, a borate ester compound, and a phosphate ester compound.

In some embodiments, the first organic solvent is selected from an aromatic-based solvent or a heteroaromatic-based solvent. Examples of the aromatic-based solvent or the heteroaromatic-based solvent include, but not limited to, 1,4-diisopropylbenzene, pentylbenzene, tetrahydronaphthalene, cyclohexylbenzene, chloronaphthalene, 1,4-dimethylnaphthalene, 3-isopropylbiphenyl, p-methylisopropylbenzene, dipentylbenzene, tripentylbenzene, pentyltoluene, 1,2-diethylbenzene, m-diethylbenzene, 1,4-diethylbenzene, 1,2,3,4-tetramethylbenzene, 1,2,3,5-tetramethylbenzene, 1,2,4,5-tetramethylbenzene, butylbenzene, dodecylbenzene, dihexylbenzene, dibutylbenzene, 1,4-diisopropylbenzene, cyclohexylbenzene, benzylbutylbenzene, dimethylnaphthalene, 3-isopropylbiphenyl, p-isopropyltoluene, 1-methylnaphthalene, 1,2,4-trichlorobenzene, 4,4′-difluorodiphenylmethane, methyl eugenol, methyl isoeugenol, diphenylmethane, 2-phenylpyridine, 3-phenylpyridine, N-methyldiphenylamine, 4-isopropylbiphenyl, 4-(3-phenylpropyl)pyridine, benzyl benzoate, 1,1-bis(3,4-dimethylphenyl)ethane, 2-isopropylnaphthalene, quinoline, isoquinoline, methyl 2-furanoate, and ethyl 2-furanoate.

In some embodiments, the first organic solvent is an aromatic ketone-based solvent. Examples of the aromatic ketone-based solvent include, but not limited to, 1-tetralone, beta-tetralone, 2-phenylepoxy-1-tetralone, 6-methoxy-1-tetralone, acetophenone, propiophenone, benzophenone, and derivatives thereof, for example, 4-methylacetophenone, 3-methylacetophenone, 2-methylacetophenone, 4-methylphenylacetone, 3-methylphenylacetone, and 2-methylphenylacetone.

In some embodiments, the first organic solvent is an aromatic ether-based solvent. Examples of the aromatic ether-based solvent include, but not limited to, 3-phenoxytoluene, butoxybenzene, 1-(dimethoxymethyl)anisole, tetrahydro-2-phenoxy-2H-pyran, methyl isoeugenol, 1,4-benzodioxan, 1,3-dipropylbenzene, 2,5-dimethoxytoluene, 4-ethylphenetole, 1,3-dipropoxybenzene, 1,2,4-trimethoxybenzene, 1,3-dimethoxybenzene, glycidyl phenyl ether, dibenzyl ether, 4-tert-butylanisole, trans-anethole, 1,2-dimethoxybenzene, 1-methoxynapthalene, diphenyl ether, 2-phenoxymethyl ether, 2-phenoxytetrahydrofuran, and 2-ethoxynaphthalene.

In some embodiments, the first organic solvent is an aliphatic ketone-based solvent. Examples of the aliphatic ketone-based solvent include, but not limited to, 2-nonanone, 3-nonanone, 5-nonanone, 2-decanone, 2,5-hexanedione, 2,6,8-trimethyl-4-nonanone, fenchone, phorone, isophorone, and 6-undecanone.

In some embodiments, the first organic solvent is an aliphatic ether-based solvent. Examples of the aliphatic ether-based solvent include, but not limited to, pentyl ether, hexyl ether, dioctyl ether, ethylene glycol dibutyl ether, diethylene glycol diethyl ether, diethylene glycol butyl methyl ether, diethylene glycol dibutyl ether, triethylene glycol dimethyl ether, triethylene glycol ethyl methyl ether, triethylene glycol butyl methyl ether, tripropylene glycol dimethyl ether, and tetraethylene glycol dimethyl ether.

In some embodiments, the first organic solvent is an ester-based solvent. Examples of the ester-based solvent include, but not limited to, alkyl octanoate, alkyl sebacate, alkyl stearate, alkyl benzoate, alkyl phenylacetate, alkyl cinnamate, alkyl oxalate, alkyl maleate, alkyl lactone, alkyl oleate, and the like. In some embodiments, the first organic solvent is selected from at least one of octyl octanoate, diethyl sebacate, diallyl phthalate, and isononyl isononanoate.

In some embodiments, the composition further includes a second organic solvent, and the second organic solvent is selected from at least one of methanol, ethanol, 2-methoxyethanol, dichloromethane, chloroform, chlorobenzene, o-dichlorobenzene, tetrahydrofuran, anisole, morpholine, toluene, o-xylene, m-xylene, p-xylene, 1,4-dioxane, acetone, 2-butanone, 1,2-dichloroethane, 3-phenoxytoluene, 1,1,1-trichloroethane, 1,1,2,2-tetrachloroethane, ethyl acetate, butyl acetate, N,N-dimethylformamide, N,N-dimethylacetamide, dimethyl sulfoxide, 1,2,3,4-tetrahydronaphthalene, decahydronaphthalene, and indene.

In some embodiments, Hansen solubility parameters of the organic solvent are as follows:
δd (dispersion force) ranges from 17.0 MPa^{1/2} to 23.2 MPa^{1/2}, for example, 18.5 MPa^{1/2} to 21.0 MPa^{1/2};
δp (polarity force) ranges from 0.2 MPa^{1/2} to 12.5 MPa^{1/2}, for example, 2.0 MPa^{1/2} to 6.0 MPa^{1/2}; and
δh (hydrogen bonding force) ranges from 0.9 MPa^{1/2} to 14.2 MPa^{1/2}, for example, 2.0 MPa^{1/2} to 6.0 MPa^{1/2}.

In the composition provided in the embodiments of the disclosure, a boiling point needs to be considered when selecting the organic solvent for the composition. In some embodiments, the boiling point of the organic solvent is greater than or equal to 150° C., greater than or equal to 180° C., greater than or equal to 200° C., greater than or equal to 250° C., or greater than or equal to 300° C. The boiling point in these ranges are beneficial to preventing nozzles of inkjet printing heads from clogging. It can be understood that, the organic solvent can be evaporated from a solvent system to form a film including a functional material.

In some embodiments, the composition is a solution.

In some embodiments, the composition is a suspension.

In some embodiments, the composition includes 0.01 wt % to 20 wt % of the organic compound or the mixture, for example, 0.1 wt % to 15 wt %, or 0.2 wt % to 10 wt %.

Some embodiments of the disclosure provide a use of the composition as a coating material or printing ink in the process for preparing an organic electronic device. In some embodiments, the composition is used to prepare the organic electronic device by a printing process or a coating process.

Examples of the printing process or the coating process include, but not limited to, inkjet printing, nozzle printing, letterpress printing, silk-screen printing, dip coating, spin-coating, scraper coating, roller printing, twist roller printing, lithographic printing, flexographic printing, rotary printing, spray coating, brush coating, transfer printing, or slit extrusion coating. In some embodiments, the printing process or the coating process is selected from intaglio printing, nozzle printing, or inkjet printing.

In some embodiments, the composition further includes one or more components such as a surfactant, a lubricant, a wetting agent, a hydrophobic agent, an adhesive, or the like, for adjusting viscosity, so as to adjust the viscosity of the composition and improve film forming performance and adhesiveness.

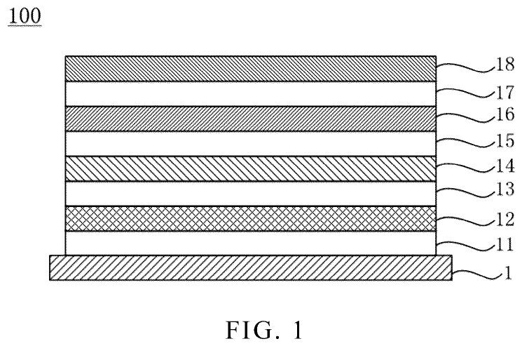

Referring to FIG. 1, some embodiments of the disclosure provide an organic electronic device 100, which includes a substrate 1, a first electrode 11, an organic functional layer, and a second electrode 18. The first electrode 11 and the second electrode 18 are disposed opposite to each other on the substrate 1. The organic functional layer is disposed between the first electrode 11 and the second electrode 18.

In some embodiments, a material of the organic functional layer includes at least one of the organic compounds described in the above-mentioned embodiments; alternatively, the material of the organic functional layer includes the mixture described in any one of the above-mentioned embodiments; and alternatively, the organic functional layer is made from the composition described in any one of the above-mentioned embodiments.

In some embodiments, the organic functional layer includes a light-emitting layer 15 disposed between the first electrode 11 and the second electrode 18. The light-emitting layer 15 includes a first host material and a guest material. The first host material includes at least one organic compound, the mixture, or the composition described in any one of the above-mentioned embodiments.

In some embodiments, the material of the light-emitting layer further includes a second host material. The second host material includes a hole transport type host material such as an aromatic amine compound. The second host material has a highest occupied molecular orbital (HOMO) energy level of −5.5 eV to −5.2 eV. The organic compound provided in the embodiments of the disclosure has better electron transport property, and the combination of the first host material and the second host material including the hole transport type host material can balance the transport of carrier, so that the energy utilization rate of the light-emitting layer is higher and the transport of the carrier is more stable. Moreover, by adjusting a mass ratio of the first host material to the second host material, the problem of biased electron transport or biased hole transport can be improved.

In some embodiments, the first electrode 11 is an anode, and the second electrode 18 is a cathode.

In some embodiments, the organic electronic device 100 includes the substrate 1, and the first electrode 11, a hole injection layer 12, a hole transport layer 13, a light-emitting auxiliary layer 14, the light-emitting layer 15, an electron transport layer 16, an electron injection layer 17, and the second electrode 18 sequentially stacked on the substrate 1.

In some embodiments, the organic electronic device 100 includes an organic light-emitting diode (OLED), an organic photovoltaic (OPV) cell, an organic light-emitting electrochemical cell (OLEEC), an organic field-effect transistor (OFET), an organic light-emitting field-effect transistor (OLEFET), an organic laser, an organic spintronic device, an organic sensor, an organic plasmonic emission diode, or the like. In some embodiments, the organic light-emitting device 100 is an organic electroluminescent device, for example, the OLED, the OLEEC, or the OLEFET.

In some embodiments, the organic electronic device 100 is the OLED, which includes the substrate 1, the first electrode 11, at least one light-emitting layer 15, and the second electrode 18.

The substrate 1 is a transparent substrate or an opaque substrate. When the substrate 1 is a transparent substrate, a transparent light-emitting device can be prepared, and methods for preparing the transparent light-emitting device can refer to methods recited in documents Bulovic et al. (Nature 1996, 380, p 29), and Gu et al. (Appl. Phys. Lett. 1996, 68, p 2606), the disclosures of which are incorporated hereby in reference by their entirety. In addition, the substrate 1 is a rigid substrate or a flexible substrate with elasticity. A material of the substrate 1 includes, but not limited to, plastics, a polymer, a metal, a semiconductor wafer, glass, or the like. In some embodiments, the substrate 1 includes at least one smooth surface, for example, the substrate 1 is a substrate without surface defects. In some embodiments, the substrate 1 is a flexible substrate, and the material of the substrate 1 includes, but not limited to, a polymer film or plastics. A glass transition temperature Tg of the material of the flexible substrate is greater than or equal to 150° C., for example, greater than or equal to 200° C., greater than or equal to 250° C., or greater than or equal to 300° C. Examples of the material of the flexible substrate include, but not limited to, polyethylene terephthalate (PET) and polyethylene naphthyl-2,6-dicarboxylate (PEN).

A material of the first electrode 11 includes at least one of a conductive metal, a conductive metal oxide, and a conductive polymer. Holes in the first electrode 11 can be injected into the hole injection layer 12, the hole transport layer 13, or the light-emitting layer 15. In some embodiments, an absolute value of a difference between work function of the first electrode 11 and HOMO energy level or valence band energy level of an emitter of the light-emitting layer 15, or between work function of the first electrode 11 and HOMO energy level or valence band energy level of a p-type semiconductor material of the hole injection layer 12, the hole transport layer 13, or the electron blocking layer, is less than 0.5 eV, for example, less than 0.3 eV, or less than 0.2 eV. Examples of the material of the first electrode 11 include, but not limited to, aluminum (Al), copper (Cu), aurum (Au), argentum (Ag), magnesium (Mg), ferrum (Fe), cobalt (Co), nickel (Ni), manganese (Mn), palladium (Pd), platinum (Pt), indium tin oxide (ITO), aluminum doped with zinc oxide (AZO), and the like. Other suitable materials of the first electrode 11 are known in the art, and can be selected for use by ordinary skilled in the art. The material of the first electrode 11 can be deposited using any suitable technology, such as a physical vapor deposition method including RF magnetron sputtering, vacuum thermal evaporation, electron beam (e-beam), or the like.

In some embodiments, the first electrode 11 is a patterned structure, such as a patterned ITO conductive substrate that can be commercially available and used to prepare the organic electronic device 100 as described above.

A material of the second electrode 18 includes at least one of a conductive metal or a conductive metal oxide. Electrons in the second electrode 18 can be injected into the electron injection layer 17, the electron transport layer 16, or the light-emitting layer 15. In some embodiments, an absolute value of a difference between work function of the second electrode 18 and LUMO energy level or valence band energy level of an emitter of the light-emitting layer 15, or between work function of the second electrode 18 and LUMO energy level or valence band energy level of a n-type semiconductor material of the electron injection layer 17, the electron transport layer 16, or the hole blocking layer, is less than 0.5 eV, for example, less than 0.3 eV, or less than 0.2 eV. All materials that can be used in the cathode of an OLED can be used as the material of the second electrode 18 of the organic electronic device of the disclosure. Examples of the material of the second electrode 18 include, but not limited to, Al, Au, Ag, calcium (Ca), barium (Ba), Mg, LiF/Al, Mg—Ag alloy, BaF₂/Al, Cu, Fe, Co, Ni, Mn, Pd, Pt, ITO, and the like. The material of the second electrode 18 can be deposited using any suitable technology, such as a suitable physical vapor deposition method including RF magnetron sputtering, vacuum thermal evaporation, electron beam (e-beam), or the like.

In some embodiments, the light-emitting layer 15 of the organic electronic device 100 is prepared from the composition as described in any one of the above-mentioned embodiments.

In some embodiments, the organic electronic device 100 has an emission wavelength of 300 nm to 1000 nm, for example, 350 nm to 900 nm, or 400 nm to 800 nm.

In some embodiments, the organic electronic device 100 is used in various electronic devices, such as a display device, an illumination device, a light source, a sensor, and the like.

In some embodiments, an electronic device including the organic electronic device 100 is a display device, an illumination device, a light source, a sensor, or the like.

The organic compounds provided in the disclosure will be described below in conjunction with preferred examples, but the organic compounds provided in the disclosure are not limited to the following examples. It should be understood that the claims summarize the scope of the disclosure. Under the guidance of the invention concept of the disclosure, those skilled in the art should be aware that certain modifications made to each example of the disclosure will be covered by the spirit and scope of the claims of the disclosure.

The following provides further detailed descriptions of the organic compounds and methods for preparing the same, based on specific examples. Raw materials used in the following examples, unless otherwise specified, are all commercially available products.

### EXAMPLES OF SYNTHESES OF ORGANIC COMPOUNDS

### Example 1

### Synthetic Route of Organic Compound P-1 is as Follows:

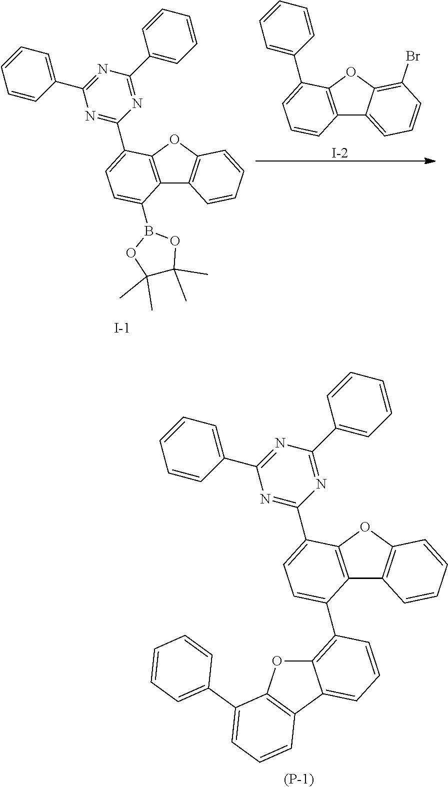

Synthesis of Organic Compound P-1: compound I-1 (0.5 mol) and compound I-2 (0.5 mol) were placed in a three-necked flask, tetratriphenylphosphine palladium (Pd(PPh₃)₄, concentration of 5%, 25 mmol), potassium carbonate (2.0 mol), toluene (2.5 L), ethanol (1.25 L), and deionized water (1.25 L) were added. The reaction system was changed with nitrogen three times, then the reaction solution was heated to 100° C. and reacted for 12 hours under a nitrogen environment. After the reaction was completed, the reaction solution was cooled naturally, washed with water, and separated to obtain an organic phase. The organic phase was dried by rotary evaporation, and purified by column chromatography to obtain the organic compound P-1 with yield of 80%. A result of electrospray ionization mass spectrometry (ESI-MS) of the organic compound P-1 was as follows: m/z [H⁺]=642.

### Example 2

### Synthetic Route of Organic Compound P-14 is as Follows:

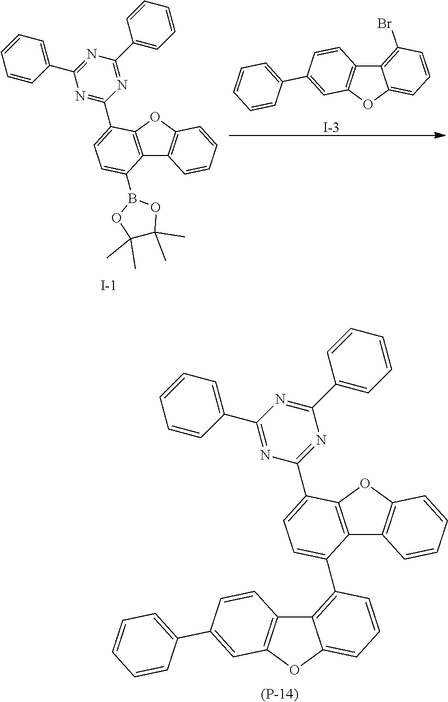

Synthesis of Organic Compound P-14: compound I-1 (0.5 mol) and compound I-3 (0.5 mol) were placed in a three-necked flask, Pd(PPh₃)₄ (concentration of 5%, 25 mmol), potassium carbonate (2.0 mol), toluene (2.5 L), ethanol (1.25 L), and deionized water (1.25 L) were added. The reaction system was changed with nitrogen three times, then the reaction solution was heated to 100° C. and reacted for 12 hours under a nitrogen environment. After the reaction was completed, the reaction solution was cooled naturally, washed with water, and separated to obtain an organic phase. The organic phase was dried by rotary evaporation, and purified by column chromatography to obtain the organic compound P-14 with yield of 78%. A result of ESI-MS of the organic compound P-14 was as follows: m/z [H⁺]=642.

### Example 3

### Synthetic Route of Organic Compound P-35 is as Follows:

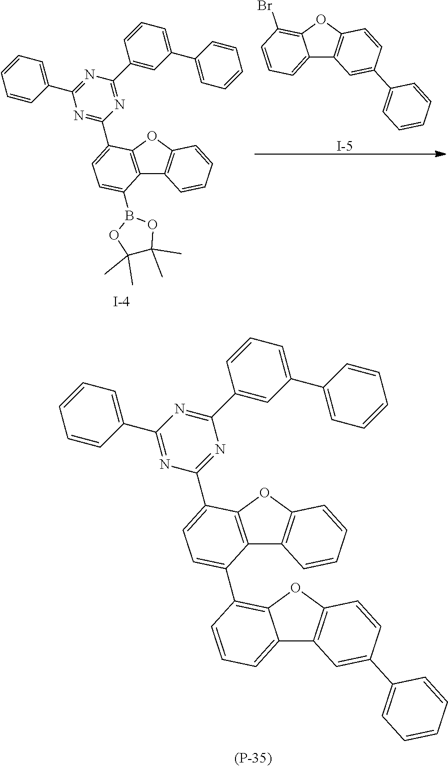

Synthesis of Organic Compound P-35: compound I-4 (0.5 mol) and compound I-5 (0.5 mol) were placed in a three-necked flask, Pd(PPh₃)₄ (concentration of 5%, 25 mmol), potassium carbonate (2.0 mol), toluene (2.5 L), ethanol (1.25 L), and deionized water (1.25 L) were added. The reaction system was changed with nitrogen three times, then the reaction solution was heated to 100° C. and reacted for 12 hours under a nitrogen environment. After the reaction was completed, the reaction solution was cooled naturally, washed with water, and separated to obtain an organic phase. The organic phase was dried by rotary evaporation, and purified by column chromatography to obtain the organic compound P-35 with yield of 82%. A result of ESI-MS of the organic compound P-35 was as follows: m/z [H⁺]=718.

### Example 4

### Synthetic Route of Organic Compound P-63 is as Follows:

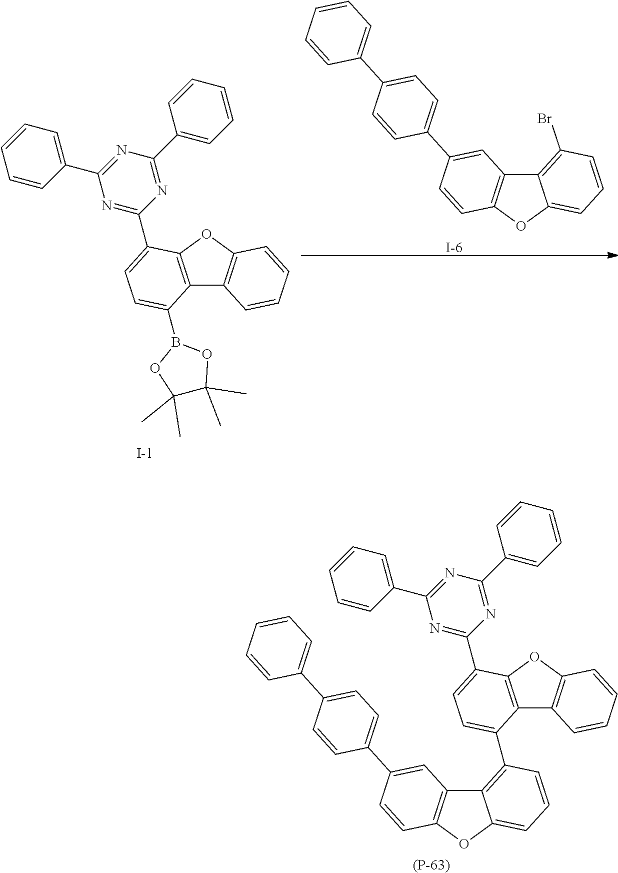

Synthesis of Organic Compound P-63: compound I-1 (0.5 mol) and compound I-6 (0.5 mol) were placed in a three-necked flask, Pd(PPh₃)₄ (concentration of 5%, 25 mmol), potassium carbonate (2.0 mol), toluene (2.5 L), ethanol (1.25 L), and deionized water (1.25 L) were added. The reaction system was changed with nitrogen three times, then the reaction solution was heated to 100° C. and reacted for 12 hours under a nitrogen environment. After the reaction was completed, the reaction solution was cooled naturally, washed with water, and separated to obtain an organic phase. The organic phase was dried by rotary evaporation, and purified by column chromatography to obtain the organic compound P-63 with yield of 73%. A result of ESI-MS of the organic compound P-63 was as follows: m/z [H⁺]=718.

### Example 5

### Synthetic Route of Organic Compound P-116 is as Follows:

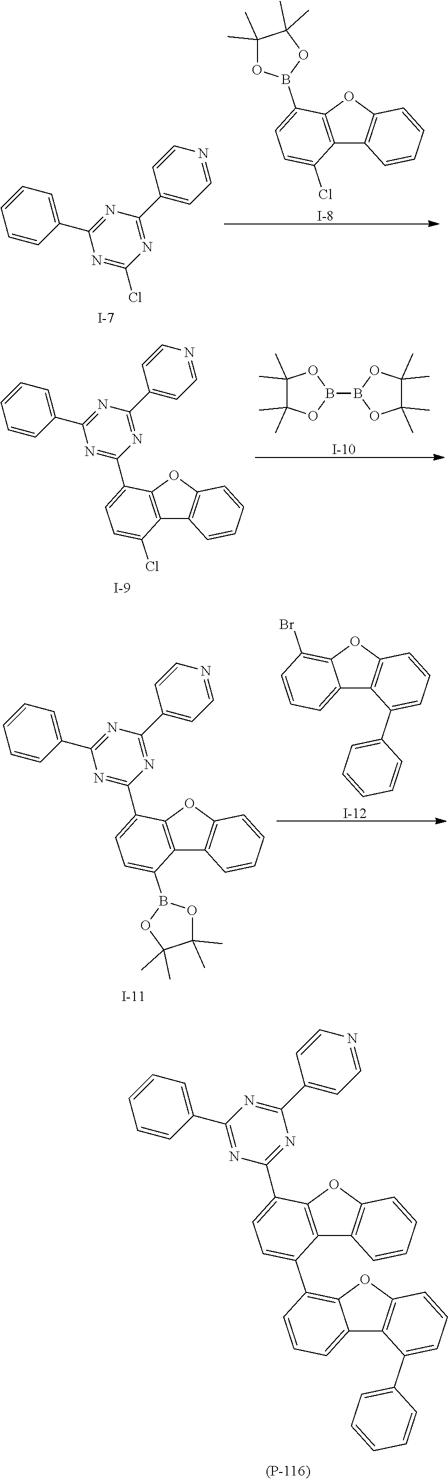

(1) Synthesis of Intermediate I-9: compound I-7 (0.8 mol) and compound I-8 (0.8 mol) were placed in a three-necked flask, Pd(PPh₃)₄ (concentration of 5%, 40 mmol), potassium carbonate (3.2 mol), toluene (4.0 L), ethanol (1.0 L), and deionized water (1.0 L) were added. The reaction system was changed with nitrogen three times, then the reaction solution was heated to 100° C. and reacted for 10 hours under a nitrogen environment. After the reaction was completed, the reaction solution was cooled naturally, washed with water, and separated to obtain an organic phase. The organic phase was dried by rotary evaporation, and purified by column chromatography to obtain the intermediate I-9 with yield of 75%. A result of ESI-MS of the intermediate I-9 was as follows: m/z [H⁺]=435.

(2) Synthesis of Intermediate I-11: intermediate I-9 (0.5 mol) and compound I-10 (0.6 mol) were placed in a three-necked flask, [1,1′-bis(diphenylphosphino)ferrocene]dichloropalladium(II) (Pd(dppf)Cl₂, 10 mmol) and potassium acetate (2.0 mol) were dissolved in 1,4-dioxane and added to the three-necked flask. The reaction system was changed with nitrogen three times, then the reaction solution was heated to 100° C. and reacted for 6 hours under a nitrogen environment. After the reaction was completed, the reaction solution was cooled naturally, washed with water, and separated to obtain an organic phase. The organic phase was dried by rotary evaporation, and purified by column chromatography to obtain the intermediate I-11 with yield of 86%. A result of ESI-MS of the intermediate I-11 was as follows: m/z [H⁺]=526.

(3) Synthesis of Organic Compound P-116: intermediate I-11 (0.4 mol) and compound I-12 (0.4 mol) were placed in a three-necked flask, Pd(PPh₃)₄ (concentration of 5%, 22 mmol), potassium carbonate (1.2 mol), toluene (2.0 L), ethanol (1.0 L), and deionized water (1.0 L) were added. The reaction system was changed with nitrogen three times, then the reaction solution was heated to 100° C. and reacted for 12 hours under a nitrogen environment. After the reaction was completed, the reaction solution was cooled naturally, washed with water, and separated to obtain an organic phase. The organic phase was dried by rotary evaporation, and purified by column chromatography to obtain the organic compound P-116 with yield of 85%. A result of ESI-MS of the organic compound P-116 was as follows: m/z [H⁺]=643.

### Example 6

### Synthetic Route of Organic Compound P-135 is as Follows:

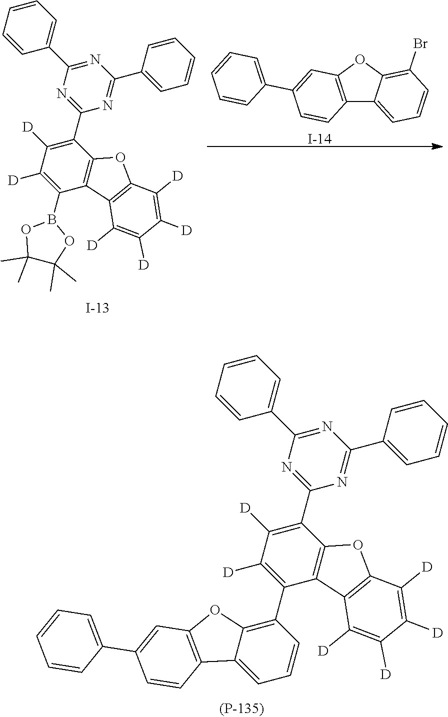

Synthesis of Organic Compound P-135: compound I-13 (0.5 mol) and compound I-14 (0.5 mol) were placed in a three-necked flask, Pd(PPh₃)₄ (concentration of 5%, 25 mmol), potassium carbonate (2.0 mol), toluene (2.5 L), ethanol (1.25 L), and deionized water (1.25 L) were added. The reaction system was changed with nitrogen three times, then the reaction solution was heated to 100° C. and reacted for 12 hours under a nitrogen environment. After the reaction was completed, the reaction solution was cooled naturally, washed with water, and separated to obtain an organic phase. The organic phase was dried by rotary evaporation, and purified by column chromatography to obtain the organic compound P-135 with yield of 82%. A result of ESI-MS of the organic compound P-135 was as follows: m/z [H⁺]=648.

### Example 7

### Synthetic Route of Organic Compound P-146 is as Follows:

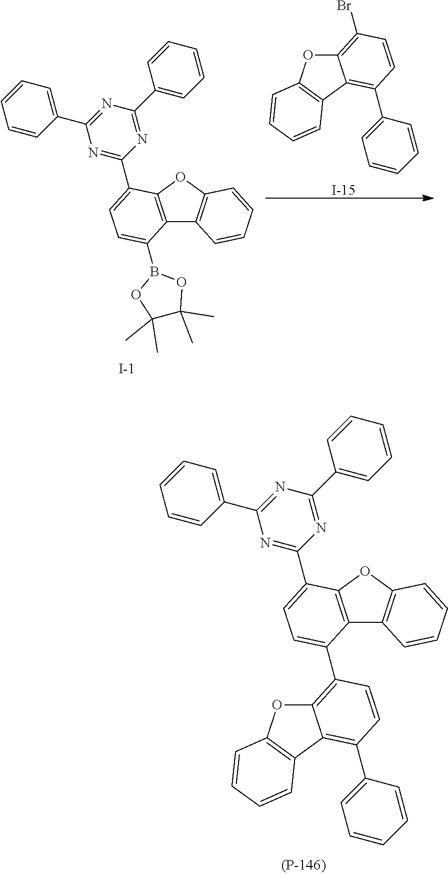

Synthesis of Organic Compound P-146: compound I-1 (0.5 mol) and compound I-15 (0.5 mol) were placed in a three-necked flask, Pd(PPh₃)₄ (concentration of 5%, 25 mmol), potassium carbonate (2.0 mol), toluene (2.5 L), ethanol (1.25 L), and deionized water (1.25 L) were added. The reaction system was changed with nitrogen three times, then the reaction solution was heated to 100° C. and reacted for 12 hours under a nitrogen environment. After the reaction was completed, the reaction solution was cooled naturally, washed with water, and separated to obtain an organic phase. The organic phase was dried by rotary evaporation, and purified by column chromatography to obtain the organic compound P-146 with yield of 77%. A result of ESI-MS of the organic compound P-146 was as follows: m/z [H⁺]=642.

### Example 8

### Synthetic Route of Organic Compound P-158 is as Follows:

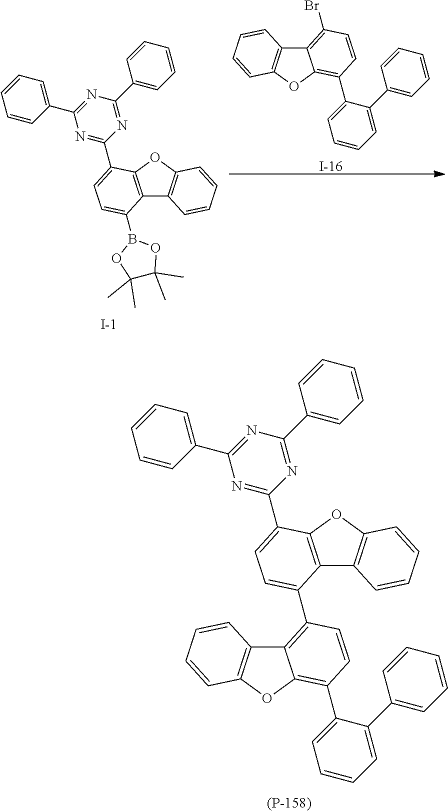

Synthesis of Organic Compound P-158: compound I-1 (0.5 mol) and compound I-16 (0.5 mol) were placed in a three-necked flask, Pd(PPh₃)₄ (concentration of 5%, 25 mmol), potassium carbonate (2.0 mol), toluene (2.5 L), ethanol (1.25 L), and deionized water (1.25 L) were added. The reaction system was changed with nitrogen three times, then the reaction solution was heated to 100° C. and reacted for 12 hours under a nitrogen environment. After the reaction was completed, the reaction solution was cooled naturally, washed with water, and separated to obtain an organic phase. The organic phase was dried by rotary evaporation, and purified by column chromatography to obtain the organic compound P-158 with yield of 75%. A result of ESI-MS of the organic compound P-158 was as follows: m/z [H⁺]=718.

### Example 9

### Synthetic Route of Organic Compound Q-3 is as Follows:

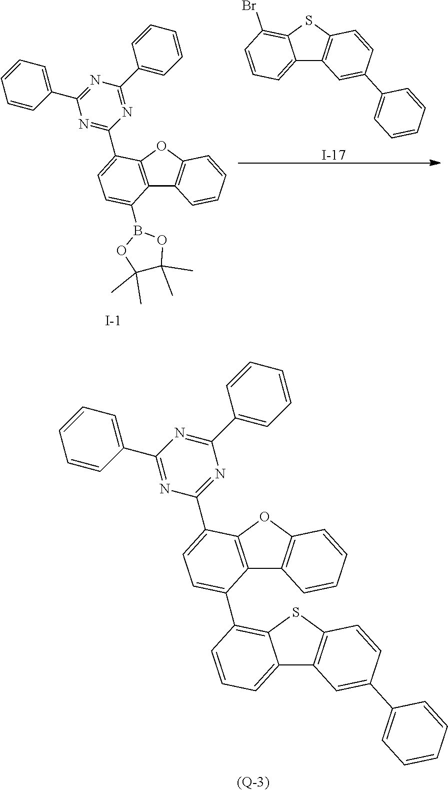

Synthesis of Organic Compound Q-3: compound I-1 (0.5 mol) and compound I-17 (0.5 mol) were placed in a three-necked flask, Pd(PPh₃)₄ (concentration of 5%, 25 mmol), potassium carbonate (2.0 mol), toluene (2.5 L), ethanol (1.25 L), and deionized water (1.25 L) were added. The reaction system was changed with nitrogen three times, then the reaction solution was heated to 100° C. and reacted for 12 hours under a nitrogen environment. After the reaction was completed, the reaction solution was cooled naturally, washed with water, and separated to obtain an organic phase. The organic phase was dried by rotary evaporation, and purified by column chromatography to obtain the organic compound Q-3 with yield of 88%. A result of ESI-MS of the organic compound Q-3 was as follows: m/z [H⁺]=658.

### Example 10

### Synthetic Route of Organic Compound Q-11 is as Follows:

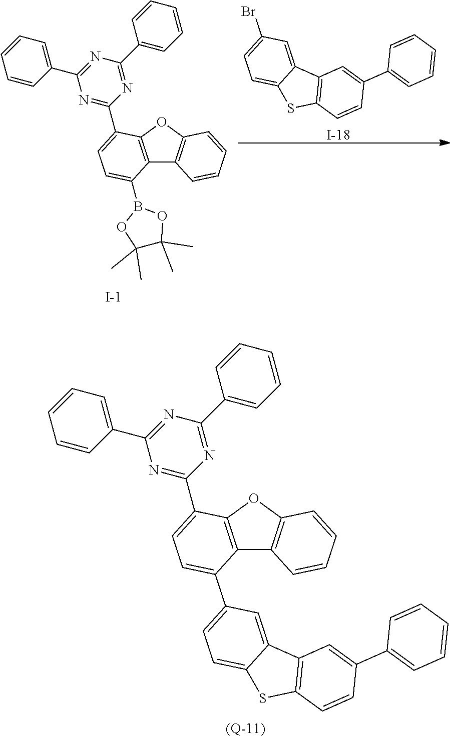

Synthesis of Organic Compound Q-11: compound I-1 (0.5 mol) and compound I-18 (0.5 mol) were placed in a three-necked flask, Pd(PPh₃)₄ (concentration of 5%, 25 mmol), potassium carbonate (2.0 mol), toluene (2.5 L), ethanol (1.25 L), and deionized water (1.25 L) were added. The reaction system was changed with nitrogen three times, then the reaction solution was heated to 100° C. and reacted for 12 hours under a nitrogen environment. After the reaction was completed, the reaction solution was cooled naturally, washed with water, and separated to obtain an organic phase. The organic phase was dried by rotary evaporation, and purified by column chromatography to obtain the organic compound Q-11 with yield of 81%. A result of ESI-MS of the organic compound Q-11 was as follows: m/z [H⁺]=658.

### Example 11

### Synthetic Route of Organic Compound Q-17 is as Follows:

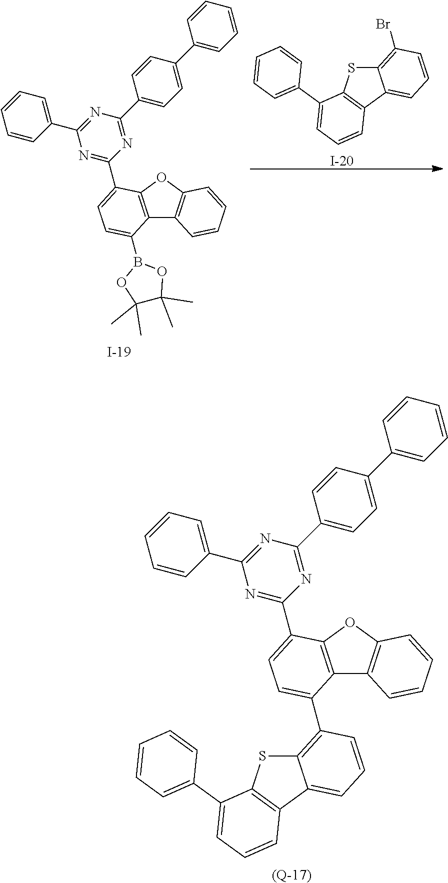

Synthesis of Organic Compound Q-17: compound I-19 (0.5 mol) and compound I-20 (0.5 mol) were placed in a three-necked flask, Pd(PPh₃)₄ (concentration of 5%, 25 mmol), potassium carbonate (2.0 mol), toluene (2.5 L), ethanol (1.25 L), and deionized water (1.25 L) were added. The reaction system was changed with nitrogen three times, then the reaction solution was heated to 100° C. and reacted for 12 hours under a nitrogen environment. After the reaction was completed, the reaction solution was cooled naturally, washed with water, and separated to obtain an organic phase. The organic phase was dried by rotary evaporation, and purified by column chromatography to obtain the organic compound Q-17 with yield of 85%. A result of ESI-MS of the organic compound Q-17 was as follows: m/z [H⁺]=734.

### Example 12

### Synthetic Route of Organic Compound Q-49 is as Follows:

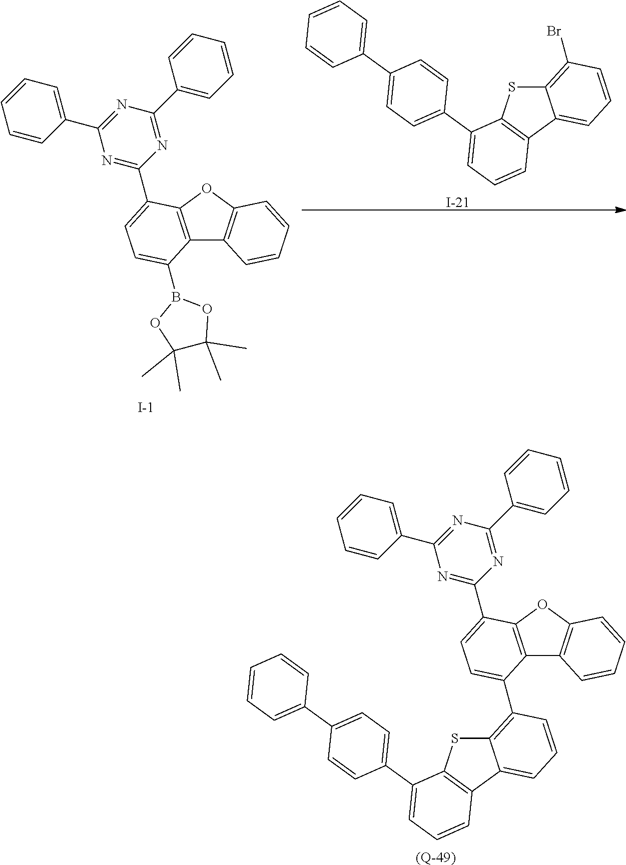

Synthesis of Organic Compound Q-49: compound I-1 (0.5 mol) and compound I-21 (0.5 mol) were placed in a three-necked flask, Pd(PPh₃)₄ (concentration of 5%, 25 mmol), potassium carbonate (2.0 mol), toluene (2.5 L), ethanol (1.25 L), and deionized water (1.25 L) were added. The reaction system was changed with nitrogen three times, then the reaction solution was heated to 100° C. and reacted for 12 hours under a nitrogen environment. After the reaction was completed, the reaction solution was cooled naturally, washed with water, and separated to obtain an organic phase. The organic phase was dried by rotary evaporation, and purified by column chromatography to obtain the organic compound Q-49 with yield of 91%. A result of ESI-MS of the organic compound Q-49 was as follows: m/z [H⁺]=734.

### Example 13

### Synthetic Route of Organic Compound Q-81 is as Follows:

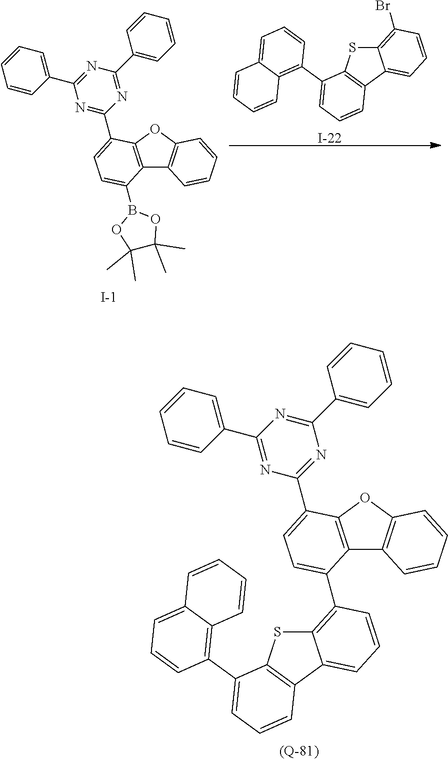

Synthesis of Organic Compound Q-81: compound I-1 (0.5 mol) and compound I-22 (0.5 mol) were placed in a three-necked flask, Pd(PPh₃)₄ (concentration of 5%, 25 mmol), potassium carbonate (2.0 mol), toluene (2.5 L), ethanol (1.25 L), and deionized water (1.25 L) were added. The reaction system was changed with nitrogen three times, then the reaction solution was heated to 100° C. and reacted for 12 hours under a nitrogen environment. After the reaction was completed, the reaction solution was cooled naturally, washed with water, and separated to obtain an organic phase. The organic phase was dried by rotary evaporation, and purified by column chromatography to obtain the organic compound Q-81 with yield of 86%. A result of ESI-MS of the organic compound Q-81 was as follows: m/z [H⁺]=708.

### Example 14

### Synthetic Route of Organic Compound Q-102 is as Follows:

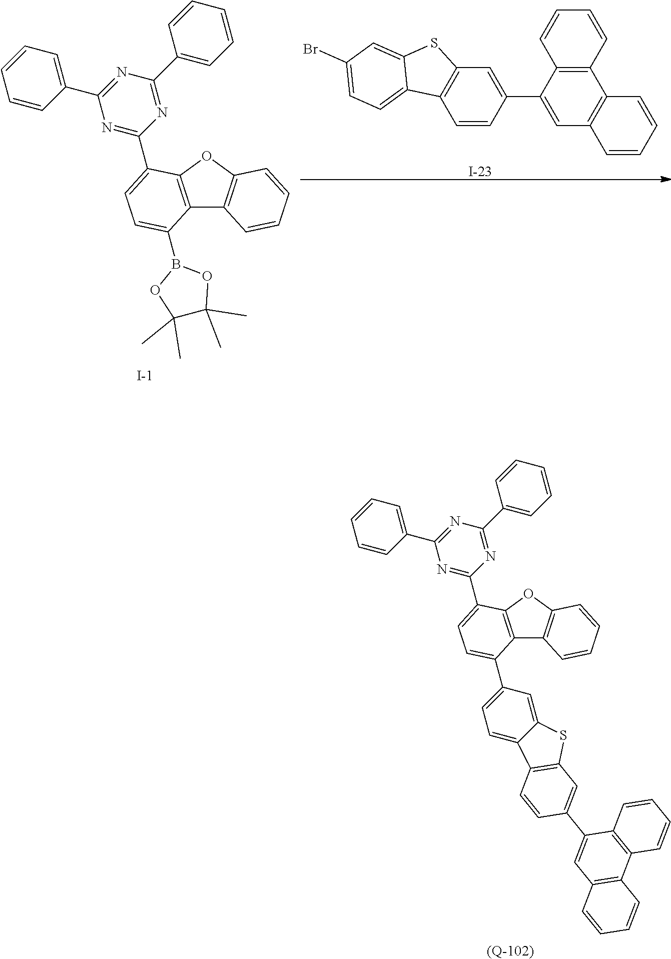

Synthesis of Organic Compound Q-102: compound I-1 (0.5 mol) and compound I-23 (0.5 mol) were placed in a three-necked flask, Pd(PPh₃)₄ (concentration of 5%, 25 mmol), potassium carbonate (2.0 mol), toluene (2.5 L), ethanol (1.25 L), and deionized water (1.25 L) were added. The reaction system was changed with nitrogen three times, then the reaction solution was heated to 100° C. and reacted for 12 hours under a nitrogen environment. After the reaction was completed, the reaction solution was cooled naturally, washed with water, and separated to obtain an organic phase. The organic phase was dried by rotary evaporation, and purified by column chromatography to obtain the organic compound Q-102 with yield of 81%. A result of ESI-MS of the organic compound Q-102 was as follows: m/z [H⁺]=758.

### Example 15

### Synthetic Route of Organic Compound Q-124 is as Follows:

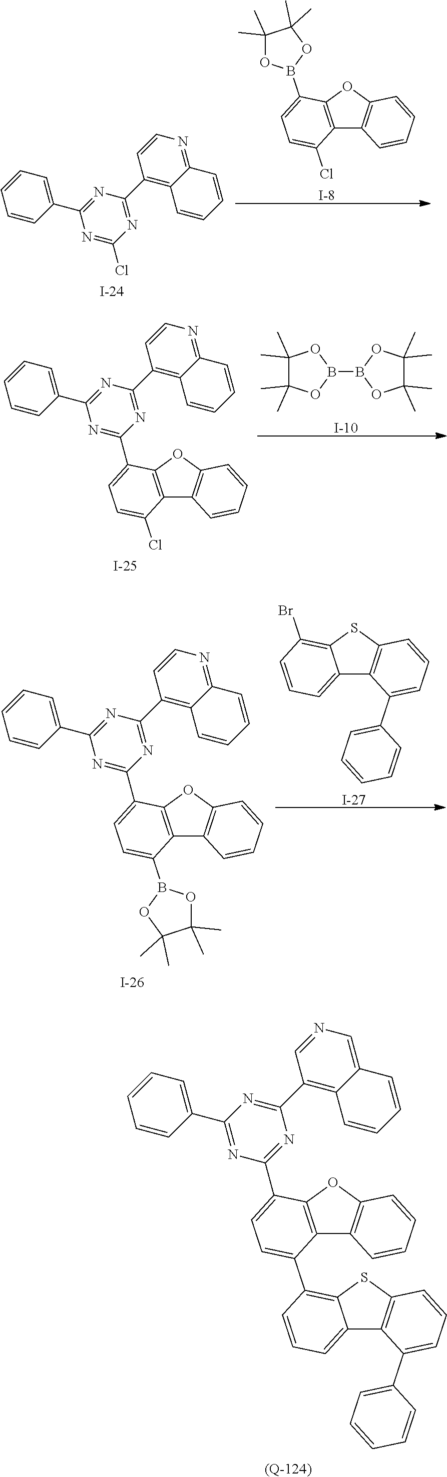

(1) Synthesis of Intermediate I-25: compound I-24 (0.8 mol) and compound I-8 (0.8 mol) were placed in a three-necked flask, Pd(PPh₃)₄ (concentration of 5%, 40 mmol), potassium carbonate (3.2 mol), toluene (4.0 L), ethanol (1.0 L), and deionized water (1.0 L) were added. The reaction system was changed with nitrogen three times, then the reaction solution was heated to 100° C. and reacted for 10 hours under a nitrogen environment. After the reaction was completed, the reaction solution was cooled naturally, washed with water, and separated to obtain an organic phase. The organic phase was dried by rotary evaporation, and purified by column chromatography to obtain the intermediate I-25 with yield of 71%. A result of ESI-MS of the intermediate I-25 was as follows: m/z [H⁺]=485.

(2) Synthesis of Intermediate I-26: intermediate I-25 (0.5 mol) and compound I-10 (0.6 mol) were placed in a three-necked flask, Pd(dppf)Cl₂ (10 mmol) and potassium acetate (2.0 mol) were dissolved in 1,4-dioxane and added to the three-necked flask. The reaction system was changed with nitrogen three times, then the reaction solution was heated to 100° C. and reacted for 6 hours under a nitrogen environment. After the reaction was completed, the reaction solution was cooled naturally, washed with water, and separated to obtain an organic phase. The organic phase was dried by rotary evaporation, and purified by column chromatography to obtain the intermediate I-26 with yield of 83%. A result of ESI-MS of the intermediate I-26 was as follows: m/z [H⁺]=577.

(3) Synthesis of Organic Compound Q-124: intermediate I-26 (0.4 mol) and compound I-27 (0.4 mol) were placed in a three-necked flask, Pd(PPh₃)₄ (concentration of 5%, 22 mmol), potassium carbonate (1.2 mol), toluene (2.0 L), ethanol (1.0 L), and deionized water (1.0 L) were added. The reaction system was changed with nitrogen three times, then the reaction solution was heated to 100° C. and reacted for 12 hours under a nitrogen environment. After the reaction was completed, the reaction solution was cooled naturally, washed with water, and separated to obtain an organic phase. The organic phase was dried by rotary evaporation, and purified by column chromatography to obtain the organic compound Q-124 with yield of 67%. A result of ESI-MS of the organic compound Q-124 was as follows: m/z [H⁺]=709.

### Example 16

### Synthetic Route of Organic Compound Q-150 is as Follows:

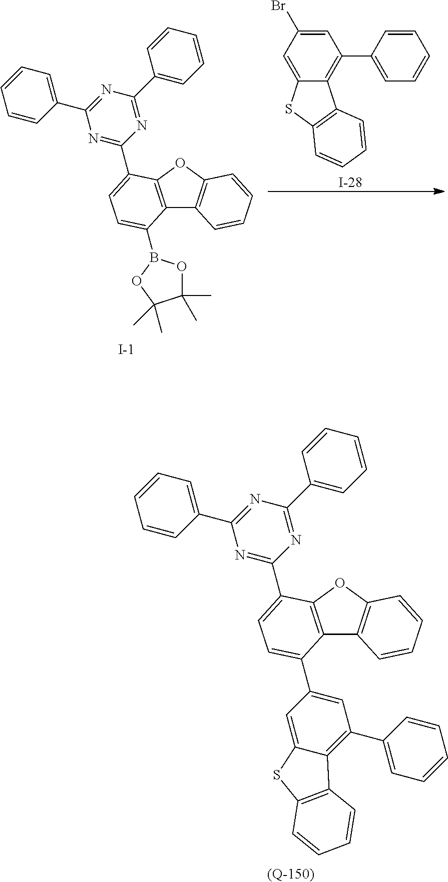

Synthesis of Organic Compound Q-150: compound I-1 (0.5 mol) and compound I-28 (0.5 mol) were placed in a three-necked flask, Pd(PPh₃)₄ (concentration of 5%, 25 mmol), potassium carbonate (2.0 mol), toluene (2.5 L), ethanol (1.25 L), and deionized water (1.25 L) were added. The reaction system was changed with nitrogen three times, then the reaction solution was heated to 100° C. and reacted for 12 hours under a nitrogen environment. After the reaction was completed, the reaction solution was cooled naturally, washed with water, and separated to obtain an organic phase. The organic phase was dried by rotary evaporation, and purified by column chromatography to obtain the organic compound Q-150 with yield of 69%. A result of ESI-MS of the organic compound Q-150 was as follows: m/z [H⁺]=658.

### Example 17

### Synthetic Route of Organic Compound R-6 is as Follows:

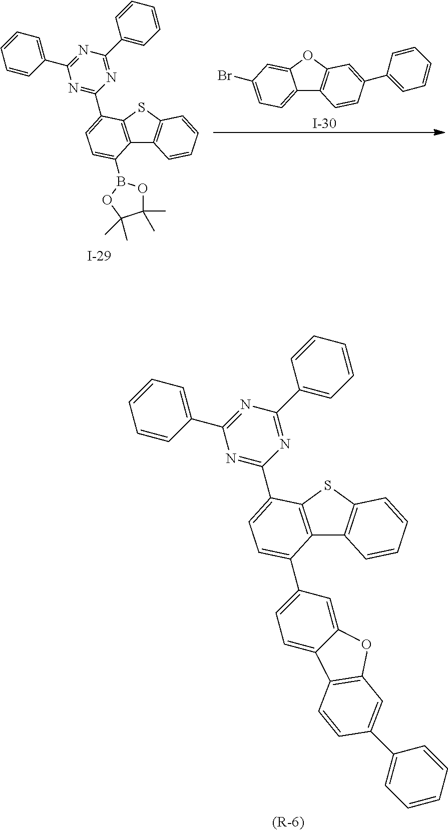

Synthesis of Organic Compound R-6: compound I-29 (0.5 mol) and compound I-30 (0.5 mol) were placed in a three-necked flask, Pd(PPh₃)₄ (concentration of 5%, 25 mmol), potassium carbonate (2.0 mol), toluene (2.5 L), ethanol (1.25 L), and deionized water (1.25 L) were added. The reaction system was changed with nitrogen three times, then the reaction solution was heated to 100° C. and reacted for 12 hours under a nitrogen environment. After the reaction was completed, the reaction solution was cooled naturally, washed with water, and separated to obtain an organic phase. The organic phase was dried by rotary evaporation, and purified by column chromatography to obtain the organic compound R-6 with yield of 87%. A result of ESI-MS of the organic compound R-6 was as follows: m/z [H⁺]=658.

### Example 18

### Synthetic Route of Organic Compound R-16 is as Follows:

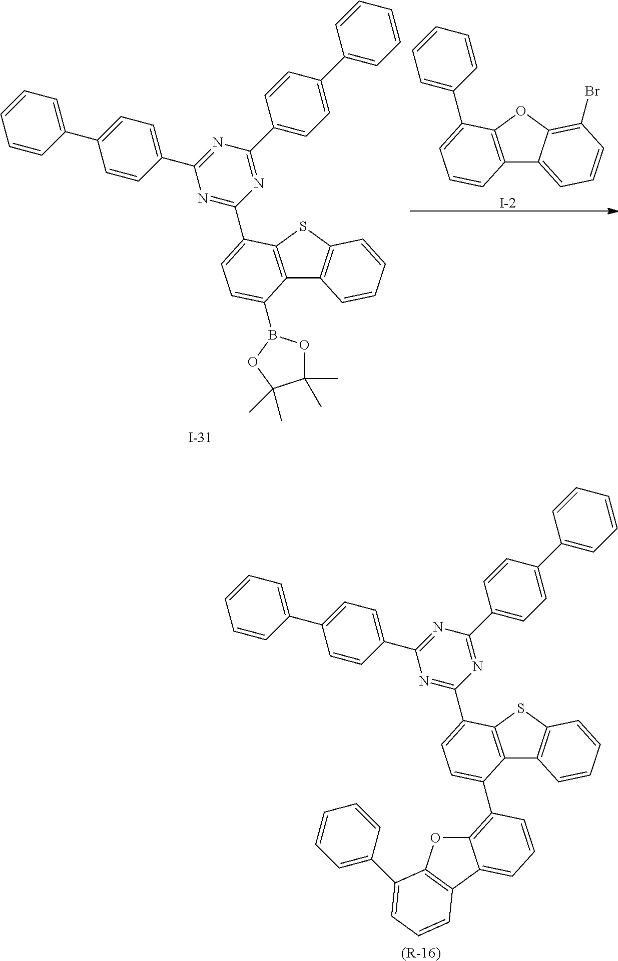

Synthesis of Organic Compound R-16: compound I-31 (0.5 mol) and compound I-2 (0.5 mol) were placed in a three-necked flask, Pd(PPh₃)₄ (concentration of 5%, 25 mmol), potassium carbonate (2.0 mol), toluene (2.5 L), ethanol (1.25 L), and deionized water (1.25 L) were added. The reaction system was changed with nitrogen three times, then the reaction solution was heated to 100° C. and reacted for 12 hours under a nitrogen environment. After the reaction was completed, the reaction solution was cooled naturally, washed with water, and separated to obtain an organic phase. The organic phase was dried by rotary evaporation, and purified by column chromatography to obtain the organic compound R-16 with yield of 88%. A result of ESI-MS of the organic compound R-16 was as follows: m/z [H⁺]=810.

### Example 19

### Synthetic Route of Organic Compound R-27 is as Follows:

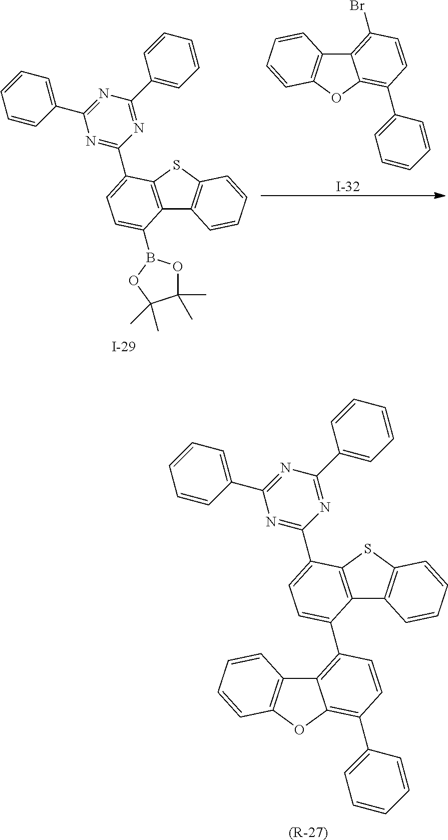

Synthesis of Organic Compound R-27: compound I-29 (0.5 mol) and compound I-32 (0.5 mol) were placed in a three-necked flask, Pd(PPh₃)₄ (concentration of 5%, 25 mmol), potassium carbonate (2.0 mol), toluene (2.5 L), ethanol (1.25 L), and deionized water (1.25 L) were added. The reaction system was changed with nitrogen three times, then the reaction solution was heated to 100° C. and reacted for 12 hours under a nitrogen environment. After the reaction was completed, the reaction solution was cooled naturally, washed with water, and separated to obtain an organic phase. The organic phase was dried by rotary evaporation, and purified by column chromatography to obtain the organic compound R-27 with yield of 91%. A result of ESI-MS of the organic compound R-27 was as follows: m/z [H⁺]=658.

### Example 20

### Synthetic Route of Organic Compound R-33 is as Follows:

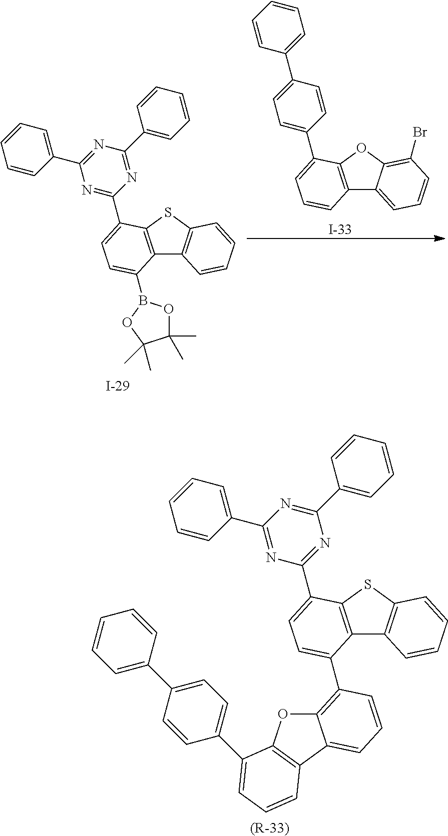

Synthesis of Organic Compound R-33: compound I-29 (0.5 mol) and compound I-33 (0.5 mol) were placed in a three-necked flask, Pd(PPh₃)₄ (concentration of 5%, 25 mmol), potassium carbonate (2.0 mol), toluene (2.5 L), ethanol (1.25 L), and deionized water (1.25 L) were added. The reaction system was changed with nitrogen three times, then the reaction solution was heated to 100° C. and reacted for 12 hours under a nitrogen environment. After the reaction was completed, the reaction solution was cooled naturally, washed with water, and separated to obtain an organic phase. The organic phase was dried by rotary evaporation, and purified by column chromatography to obtain the organic compound R-33 with yield of 84%. A result of ESI-MS of the organic compound R-33 was as follows: m/z [H⁺]=734.

### Example 21

### Synthetic Route of Organic Compound R-52 is as Follows:

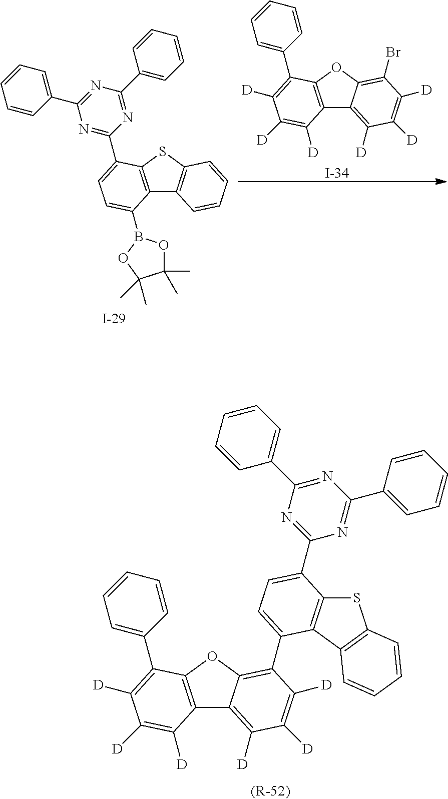

Synthesis of Organic Compound R-52: compound I-29 (0.5 mol) and compound I-34 (0.5 mol) were placed in a three-necked flask, Pd(PPh₃)₄ (concentration of 5%, 25 mmol), potassium carbonate (2.0 mol), toluene (2.5 L), ethanol (1.25 L), and deionized water (1.25 L) were added. The reaction system was changed with nitrogen three times, then the reaction solution was heated to 100° C. and reacted for 12 hours under a nitrogen environment. After the reaction was completed, the reaction solution was cooled naturally, washed with water, and separated to obtain an organic phase. The organic phase was dried by rotary evaporation, and purified by column chromatography to obtain the organic compound R-52 with yield of 92%. A result of ESI-MS of the organic compound R-52 was as follows: m/z [H⁺]=664.

### Example 22

### Synthetic Route of Organic Compound R-72 is as Follows:

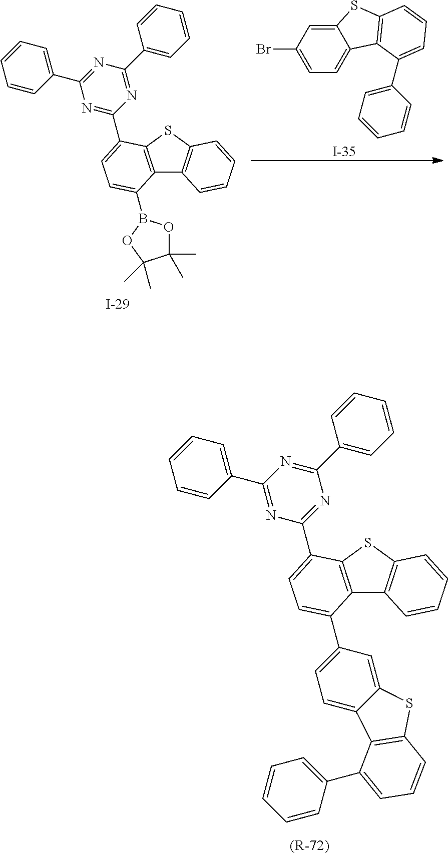

Synthesis of Organic Compound R-72: compound I-29 (0.5 mol) and compound I-35 (0.5 mol) were placed in a three-necked flask, Pd(PPh₃)₄ (concentration of 5%, 25 mmol), potassium carbonate (2.0 mol), toluene (2.5 L), ethanol (1.25 L), and deionized water (1.25 L) were added. The reaction system was changed with nitrogen three times, then the reaction solution was heated to 100° C. and reacted for 12 hours under a nitrogen environment. After the reaction was completed, the reaction solution was cooled naturally, washed with water, and separated to obtain an organic phase. The organic phase was dried by rotary evaporation, and purified by column chromatography to obtain the organic compound R-72 with yield of 86%. A result of ESI-MS of the organic compound R-72 was as follows: m/z [H⁺]=674.

### Example 23

### Synthetic Route of Organic Compound R-95 is as Follows:

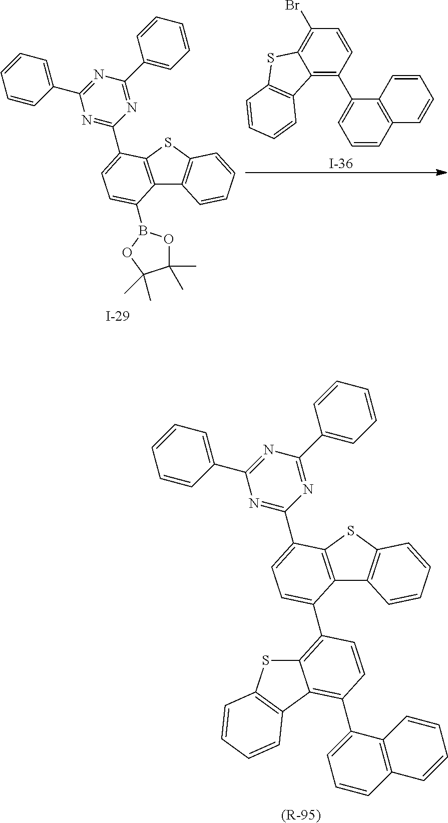

Synthesis of Organic Compound R-95: compound I-29 (0.5 mol) and compound I-36 (0.5 mol) were placed in a three-necked flask, Pd(PPh₃)₄ (concentration of 5%, 25 mmol), potassium carbonate (2.0 mol), toluene (2.5 L), ethanol (1.25 L), and deionized water (1.25 L) were added. The reaction system was changed with nitrogen three times, then the reaction solution was heated to 100° C. and reacted for 12 hours under a nitrogen environment. After the reaction was completed, the reaction solution was cooled naturally, washed with water, and separated to obtain an organic phase. The organic phase was dried by rotary evaporation, and purified by column chromatography to obtain the organic compound R-95 with yield of 91%. A result of ESI-MS of the organic compound R-95 was as follows: m/z [H⁺]=724.

### Example 24

### Synthetic Route of Organic Compound R-121 is as Follows:

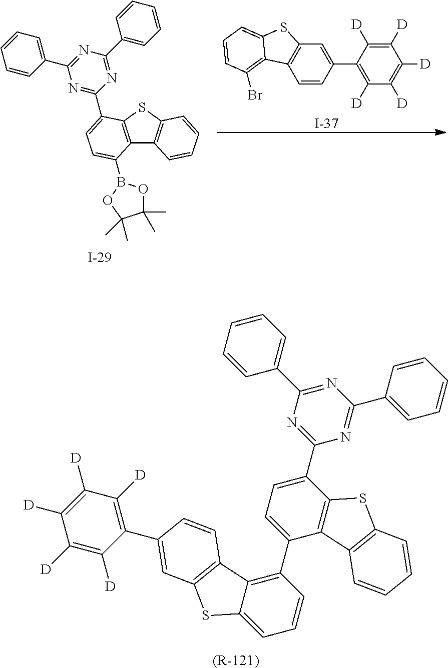

Synthesis of Organic Compound R-121: compound I-29 (0.5 mol) and compound I-37 (0.5 mol) were placed in a three-necked flask, Pd(PPh₃)₄ (concentration of 5%, 25 mmol), potassium carbonate (2.0 mol), toluene (2.5 L), ethanol (1.25 L), and deionized water (1.25 L) were added. The reaction system was changed with nitrogen three times, then the reaction solution was heated to 100° C. and reacted for 12 hours under a nitrogen environment. After the reaction was completed, the reaction solution was cooled naturally, washed with water, and separated to obtain an organic phase. The organic phase was dried by rotary evaporation, and purified by column chromatography to obtain the organic compound R-121 with yield of 85%. A result of ESI-MS of the organic compound R-121 was as follows: m/z [H⁺]=679.

### Example 25

### Synthetic Route of Organic Compound S-2 is as Follows:

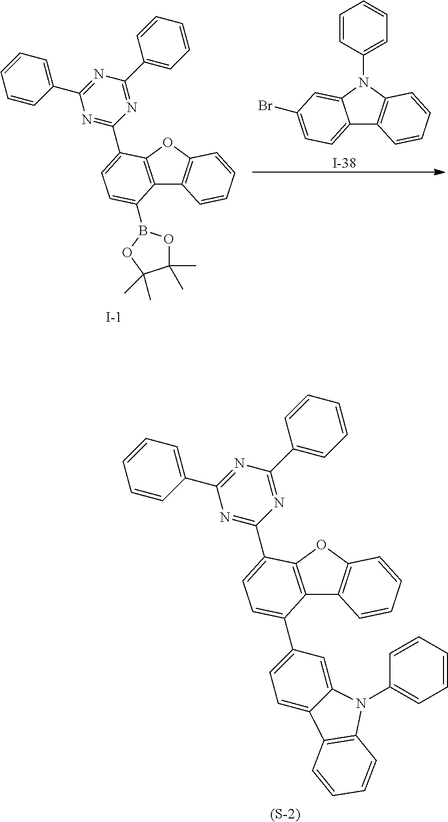

Synthesis of Organic Compound S-2: compound I-1 (0.5 mol) and compound I-38 (0.5 mol) were placed in a three-necked flask, Pd(PPh₃)₄ (concentration of 5%, 25 mmol), potassium carbonate (2.0 mol), toluene (2.5 L), ethanol (1.25 L), and deionized water (1.25 L) were added. The reaction system was changed with nitrogen three times, then the reaction solution was heated to 100° C. and reacted for 12 hours under a nitrogen environment. After the reaction was completed, the reaction solution was cooled naturally, washed with water, and separated to obtain an organic phase. The organic phase was dried by rotary evaporation, and purified by column chromatography to obtain the organic compound S-2 with yield of 88%. A result of ESI-MS of the organic compound S-2 was as follows: m/z [H⁺]=641.

### Example 26

### Synthetic Route of Organic Compound S-6 is as Follows:

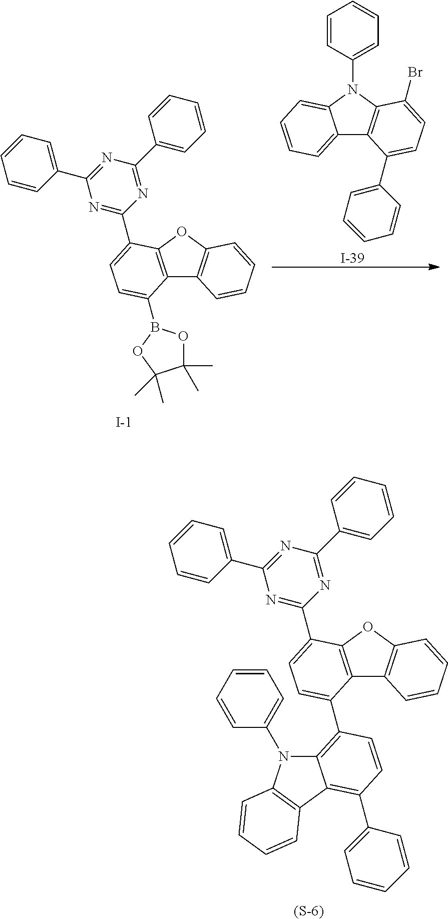

Synthesis of Organic Compound S-6: compound I-1 (0.5 mol) and compound I-39 (0.5 mol) were placed in a three-necked flask, Pd(PPh₃)₄ (concentration of 5%, 25 mmol), potassium carbonate (2.0 mol), toluene (2.5 L), ethanol (1.25 L), and deionized water (1.25 L) were added. The reaction system was changed with nitrogen three times, then the reaction solution was heated to 100° C. and reacted for 12 hours under a nitrogen environment. After the reaction was completed, the reaction solution was cooled naturally, washed with water, and separated to obtain an organic phase. The organic phase was dried by rotary evaporation, and purified by column chromatography to obtain the organic compound S-6 with yield of 56%. A result of ESI-MS of the organic compound S-6 was as follows: m/z [H⁺]=717.

### Example 27

### Synthetic Route of Organic Compound S-24 is as Follows:

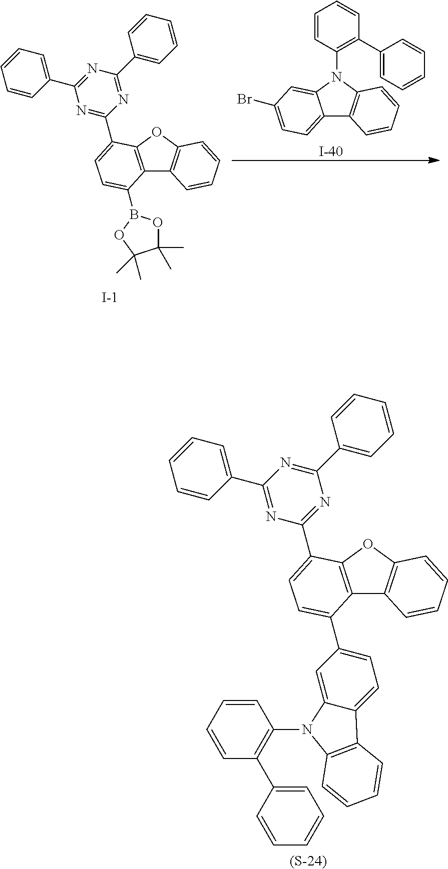

Synthesis of Organic Compound S-24: compound I-1 (0.5 mol) and compound I-40 (0.5 mol) were placed in a three-necked flask, Pd(PPh₃)₄ (concentration of 5%, 25 mmol), potassium carbonate (2.0 mol), toluene (2.5 L), ethanol (1.25 L), and deionized water (1.25 L) were added. The reaction system was changed with nitrogen three times, then the reaction solution was heated to 100° C. and reacted for 12 hours under a nitrogen environment. After the reaction was completed, the reaction solution was cooled naturally, washed with water, and separated to obtain an organic phase. The organic phase was dried by rotary evaporation, and purified by column chromatography to obtain the organic compound S-24 with yield of 81%. A result of ESI-MS of the organic compound S-24 was as follows: m/z [H⁺]=717.

### Example 28

### Synthetic Route of Organic Compound S-49 is as Follows:

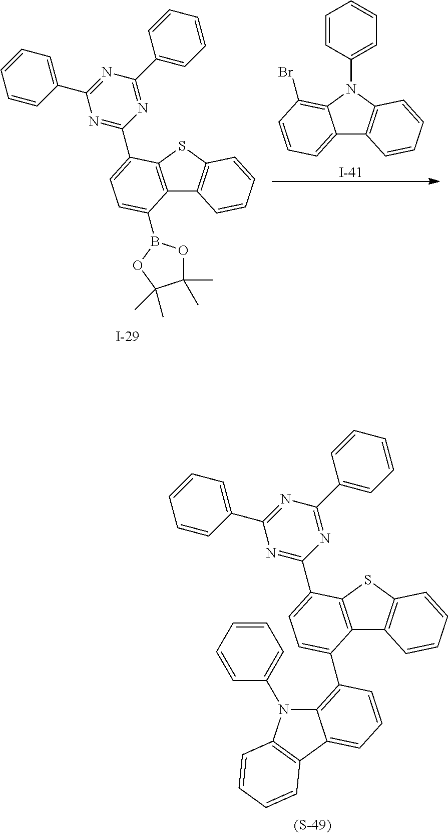

Synthesis of Organic Compound S-49: compound I-29 (0.5 mol) and compound I-41 (0.5 mol) were placed in a three-necked flask, Pd(PPh₃)₄ (concentration of 5%, 25 mmol), potassium carbonate (2.0 mol), toluene (2.5 L), ethanol (1.25 L), and deionized water (1.25 L) were added. The reaction system was changed with nitrogen three times, then the reaction solution was heated to 100° C. and reacted for 12 hours under a nitrogen environment. After the reaction was completed, the reaction solution was cooled naturally, washed with water, and separated to obtain an organic phase. The organic phase was dried by rotary evaporation, and purified by column chromatography to obtain the organic compound S-49 with yield of 83%. A result of ESI-MS of the organic compound S-49 was as follows: m/z [H⁺]=657.

### Example 29

### Synthetic Route of Organic Compound S-60 is as Follows:

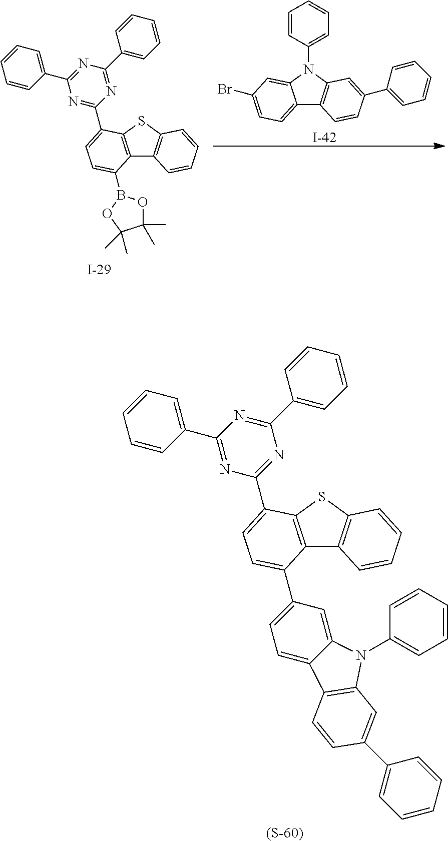

Synthesis of Organic Compound S-60: compound I-29 (0.5 mol) and compound I-42 (0.5 mol) were placed in a three-necked flask, Pd(PPh₃)₄ (concentration of 5%, 25 mmol), potassium carbonate (2.0 mol), toluene (2.5 L), ethanol (1.25 L), and deionized water (1.25 L) were added. The reaction system was changed with nitrogen three times, then the reaction solution was heated to 100° C. and reacted for 12 hours under a nitrogen environment. After the reaction was completed, the reaction solution was cooled naturally, washed with water, and separated to obtain an organic phase. The organic phase was dried by rotary evaporation, and purified by column chromatography to obtain the organic compound S-60 with yield of 89%. A result of ESI-MS of the organic compound S-60 was as follows: m/z [H⁺]=733.

### Example 30

### Synthetic Route of Organic Compound S-80 is as Follows:

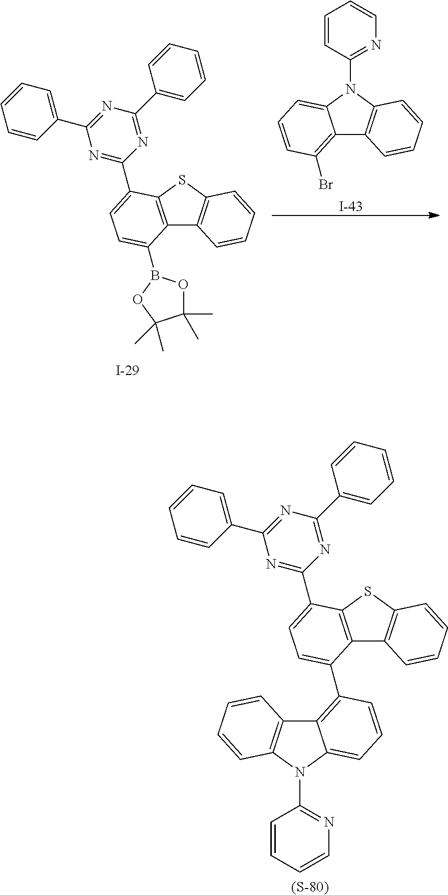

Synthesis of Organic Compound S-80: compound I-29 (0.5 mol) and compound I-43 (0.5 mol) were placed in a three-necked flask, Pd(PPh₃)₄ (concentration of 5%, 25 mmol), potassium carbonate (2.0 mol), toluene (2.5 L), ethanol (1.25 L), and deionized water (1.25 L) were added. The reaction system was changed with nitrogen three times, then the reaction solution was heated to 100° C. and reacted for 12 hours under a nitrogen environment. After the reaction was completed, the reaction solution was cooled naturally, washed with water, and separated to obtain an organic phase. The organic phase was dried by rotary evaporation, and purified by column chromatography to obtain the organic compound S-80 with yield of 86%. A result of ESI-MS of the organic compound S-80 was as follows: m/z [H⁺]=658.

### Comparative Examples

The disclosure further provides comparative examples 1 to 4, and organic compounds of the comparative examples 1 to 4 are hereafter referred to as a first comparative compound REF01, a second comparative compound REF02, a third comparative compound REF03, and a fourth comparative compound REF04, respectively. Chemical structures of REF01, REF02, REF03, and REF04 are as follows:

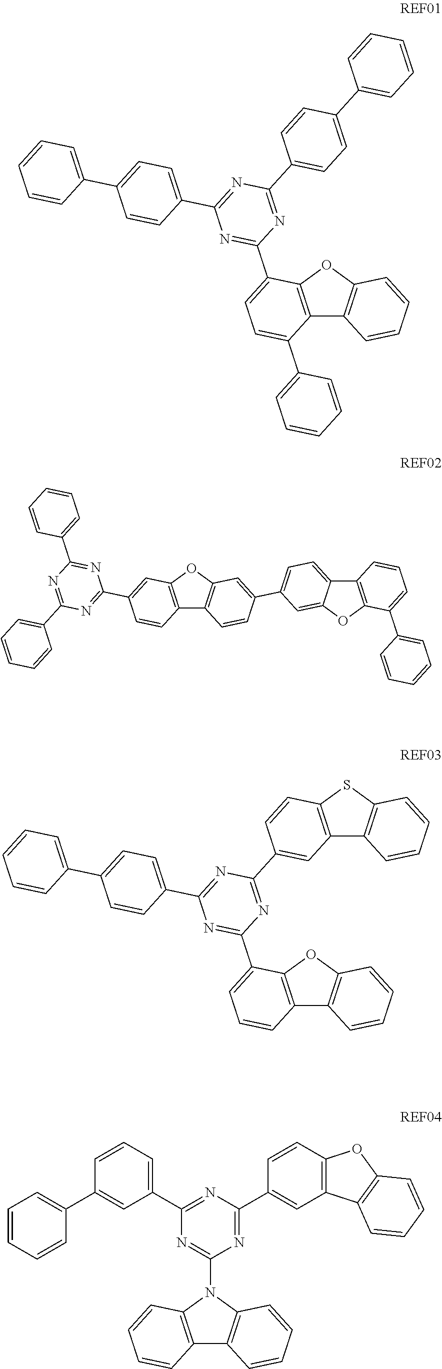 [SMILES: c1ccc(-c2ccc(-c3nc(-c4ccc(-c5ccccc5)cc4)nc(-c4ccc(-c5ccccc5)c5c4oc4ccccc45)n3)cc2)cc1 ; c1ccc(-c2nc(-c3ccccc3)nc(-c3ccc4c(c3)oc3cc(-c5ccc6c(c5)oc5c(-c7ccccc7)cccc56)ccc34)n2)cc1 ; c1ccc(-c2ccc(-c3nc(-c4ccc5sc6ccccc6c5c4)nc(-c4cccc5c4oc4ccccc45)n3)cc2)cc1 ; c1ccc(-c2cccc(-c3nc(-c4ccc5oc6ccccc6c5c4)nc(-n4c5ccccc5c5ccccc54)n3)c2)cc1]

### Preparation and Characterization of OLED Devices

The organic electronic device 100 as illustrated in FIG. 1 is an OLED device, and the method for preparing the OLED device is taken an example for illustration. The following will provide a detailed description of the method for preparing OLED devices using the organic compounds provided in the examples 1-30 of the disclosure.

In the following method for preparing the OLED devices, an ITO conductive glass was used as an anode substrate (a substrate composed of the substrate 1 and the first electrode 11), PD and HT were used as materials of the hole injection layer 12, HT was used as a material of the hole transport layer 13, Prime was used as a material of the light-emitting auxiliary layer 14, Host-1 or Host-2 was used as the second host material of the light-emitting layer 15, Dopant was used as the guest material of the light-emitting layer 15, ET and Liq were used as materials of the electron transport layer 16, Liq was used as a material of the electron injection layer 17, and Al was used as a material of the second electrode 18 (cathode). Moreover, each of the organic compounds provided in examples 1-30 was used as the first host material of the light-emitting layer 15, so as to obtain corresponding OLED device. Chemical structures of PD, HT, Prime, Host-1, Host-2, Dopant, ET, and Liq are as follows:

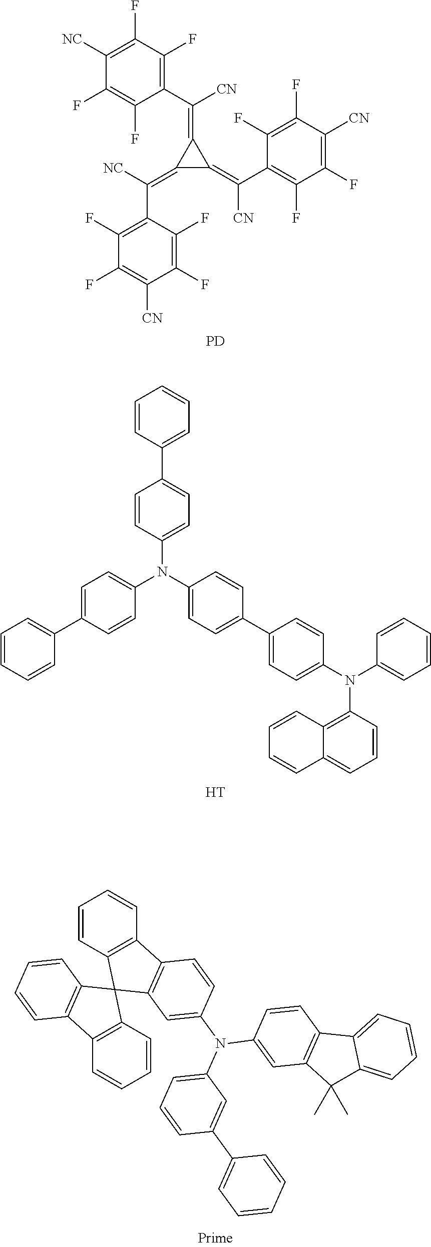

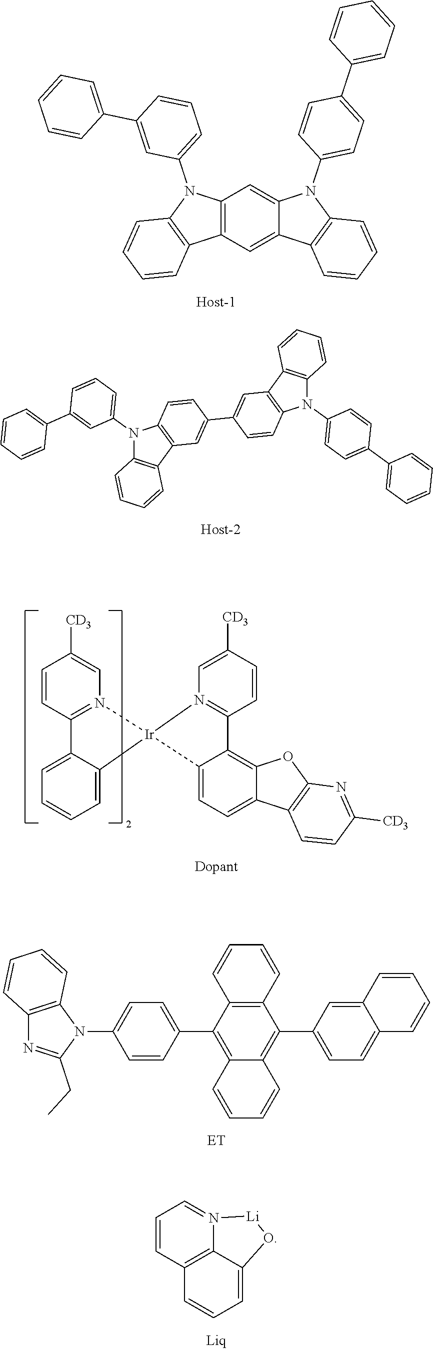

The following will provide a detailed description of the processes for preparing the OLED devices using the above-mentioned materials through specific examples.

Taking the method for preparing the OLED device using the organic compound P-1 provided in example 1 as the first host material as an example, the prepared OLED device is hereafter referred to as “an OLED-1 device”. The method for preparing the OLED-1 device includes steps as follows.

Step a, cleaning an ITO conductive glass substrate (a structure composed of the substrate 1 and the first electrode 11). Specifically, providing the ITO conductive glass substrate and performing ultrasonic cleaning with one or more cleaning agents such as deionized water, acetone, isopropanol, and chloroform, so as to improve the work function of the first electrode 11.

Step b, forming the hole injection layer 12 on the first electrode 11. Specifically, respectively depositing materials PD and HT on the first electrode 11 at a deposition rate of 1 Å/s, in which a ratio of a deposition rate of PD to a deposition rate of HT is 3:97, obtaining the hole injection layer 12 with a thickness of 30 nm.

Step c, forming the hole transport layer 13 on the hole injection layer 12. Specifically, depositing the material HT on the hole injection layer 12 at a deposition rate of 1.5 Å/s, obtaining the hole transport layer 13 with a thickness of 60 nm.

Step d, forming the light-emitting auxiliary layer 14 on the hole transport layer 13. Specifically, depositing the material Prime on the hole transport layer 13 at a deposition rate of 1 Å/s, obtaining the light-emitting auxiliary layer 14 with a thickness of 40 nm.

Step e, forming the light-emitting layer 15 on the light-emitting auxiliary layer 14. Specifically, respectively depositing materials Host-1, P-1, and Dopant on the light-emitting auxiliary layer 14 at a deposition rate of 1 Å/s, in which a ratio of a deposition rate of Host-1, to a deposition rate of P-1, and to a deposition rate of Dopant is 63:27:10, obtaining the light-emitting layer 15 with a thickness of 40 nm.

Step f, forming the electron transport layer 16 on the light-emitting layer 15. Specifically, placing materials ET and Liq in different evaporation units of vacuum chamber, and co-depositing materials ET and Liq on the light-emitting layer 15 at a weight ratio of 5:5 in high vacuum (1×10⁻⁶ millibar), obtaining the electron transport layer 16 with a thickness of 30 nm.

Step g, forming the electron injection layer 17 on the electron transport layer 16. Specifically, depositing the material Liq on the electron transport layer 16 at a deposition rate of 1 Å/s, obtaining the electron injection layer 17 with a thickness of 1 nm.

Step h, forming the second electrode 18 on the electron injection layer 17. Specifically, depositing the material Al on the electron injection layer 17 at a deposition rate of 1 Å/s, obtaining the second electrode 18 with a thickness of 100 nm.

Step i, encapsulating the device prepared above in a nitrogen glove box using UV cured resin, obtaining the OLED-1 device.

According to the method for preparing the OLED-1 device, the organic compounds provided in examples 2-30 were used as first host materials of light-emitting layers, respectively, obtaining OLED-2 to OLED-30 devices. Compared to the method for preparing the OLED-1 device, in the methods for preparing the OLED-2 to OLED-22 devices, except for the host materials that includes the first and second host materials, all other functional layers and experimental conditions are the same.

In addition, according to the method for preparing the OLED-1 device, the comparative compounds REF01 TO REF04 were used as first host materials of light-emitting layers, respectively, obtaining OLED-REF01 to OLED-REF04 devices. Compared to the method for preparing the OLED-1 device, in the methods for preparing the OLED-REF01 to OLED-REF04 devices, except for the host materials that includes the first and second host materials, all other functional layers and experimental conditions are the same.

In the disclosure, current voltage (J-V) characteristics of the OLED-1 to OLED-30 and OLED-REF01 to OLED-REF04 devices were characterized, and parameters such as voltage (V), luminous efficiency (cd/A), and service life (LT95/h) were measured, as shown in Table 1. The voltage and the luminous efficiency were measured at current density of 10 mA/cm², the service life is a time it takes for the brightness of each OLED device to decrease from the initial brightness of 1 knit to 95% of the initial brightness at current density of 50 mA/cm².

TABLE 1

| | | | | Luminous | |
|---|---|---|---|---|---|
| | First host | Second host | Voltage | efficiency | LT95 |
| Device | material | material | (V) | (cd/A) | (h) |
| OLED-1 | P-1 | Host-1 | 3.6 | 186.9 | 69 |
| OLED-2 | P-14 | Host-1 | 3.6 | 191.8 | 72 |
| OLED-3 | P-35 | Host-1 | 3.6 | 175.9 | 67 |
| OLED-4 | P-63 | Host-1 | 3.5 | 183.3 | 66 |
| OLED-5 | P-116 | Host-1 | 3.6 | 180.6 | 68 |
| OLED-6 | P-135 | Host-1 | 3.6 | 188.4 | 70 |
| OLED-7 | P-146 | Host-1 | 3.6 | 187.8 | 72 |
| OLED-8 | P-158 | Host-1 | 3.6 | 179.3 | 76 |
| OLED-9 | Q-3 | Host-1 | 3.7 | 182.6 | 71 |
| OLED-10 | Q-11 | Host-1 | 3.6 | 191.7 | 78 |
| OLED-11 | Q-17 | Host-1 | 3.6 | 190.0 | 76 |
| OLED-12 | Q-49 | Host-1 | 3.6 | 177.9 | 75 |
| OLED-13 | Q-81 | Host-1 | 3.5 | 186.1 | 77 |
| OLED-14 | Q-102 | Host-1 | 3.6 | 183.8 | 75 |
| OLED-15 | Q-124 | Host-1 | 3.6 | 189.6 | 68 |
| OLED-16 | Q-150 | Host-1 | 3.4 | 190.3 | 78 |
| OLED-17 | R-6 | Host-2 | 3.6 | 195.6 | 71 |
| OLED-18 | R-16 | Host-2 | 3.6 | 196.0 | 65 |
| OLED-19 | R-27 | Host-2 | 3.5 | 191.3 | 68 |
| OLED-20 | R-33 | Host-2 | 3.6 | 193.3 | 67 |
| OLED-21 | R-52 | Host-2 | 3.5 | 197.9 | 67 |
| OLED-22 | R-72 | Host-2 | 3.6 | 198.6 | 69 |
| OLED-23 | R-95 | Host-2 | 3.6 | 192.9 | 70 |
| OLED-24 | R-121 | Host-2 | 3.5 | 195.1 | 71 |
| OLED-25 | S-2 | Host-2 | 3.6 | 196.4 | 67 |
| OLED-26 | S-6 | Host-2 | 3.6 | 195.2 | 68 |
| OLED-27 | S-24 | Host-2 | 3.6 | 197.4 | 65 |
| OLED-28 | S-49 | Host-2 | 3.4 | 199.1 | 67 |
| OLED-29 | S-60 | Host-2 | 3.6 | 197.3 | 65 |
| OLED-30 | S-80 | Host-2 | 3.6 | 186.8 | 67 |
| OLED-REF01 | REF01 | Host-1 | 3.8 | 119.0 | 52 |
| OLED-REF02 | REF02 | Host-1 | 3.7 | 111.8 | 44 |
| OLED-REF03 | REF03 | Host-2 | 3.9 | 143.6 | 49 |
| OLED-REF04 | REF04 | Host-2 | 3.8 | 176.2 | 50 |

As can be seen from Table 1, when the organic compounds provided in examples 1 to 30 of the disclosure were used as the first host materials, the OLED devices prepared using the combination of the first host materials and the second material Host-1 or Host-2 have lower voltage, higher luminous efficiency, and longer service life. Compared with the combination of the comparative compounds REF01 to REF04 and the second material Host-1 or Host-2, the combination of the organic compounds provided in examples 1 to 30 of the disclosure and the second material Host-1 or Host-2 can achieve better energy transfer from the host material to the guest material. Therefore, when each of the organic compounds provided in examples 1 to 30 and the second host material Host-1 or Host-2 are used as the host materials to form a green light-emitting layer by co-evaporation, driving voltage, luminous efficiency, and service life of the OLED device including the green light-emitting layer can be improved.

Some embodiments of the disclosure further provide a display panel including the organic electronic device described in any one of the above-mentioned embodiments.

In the embodiments of the disclosure, the structure of the organic compound is composed of multiple conjugated planar rigid functional groups rich in electrons, such as a heterocyclic ring containing N, and dibenzoheterocyclic rings with at least one heteroatom of O and S, so that the organic compound has stronger electron transport property and structural stability. When the organic compound is used in a light-emitting layer in combination with a hole transport type host material, effective transport of excited state energy can be achieved, which is beneficial to improving luminous efficiency and service life of an organic electronic device using the organic compound. In addition, when Z¹, Z², and Z³ are all N, the heterocyclic ring containing N is directly connected to the no. 4 site of a first dibenzoheterocyclic ring, and the no. 1 site of the first dibenzoheterocyclic ring is connected to a second dibenzoheterocyclic ring. The double substitution of the first dibenzoheterocyclic ring makes the molecular conformation of the organic compound more compact, and a dihedral angle between a plane of the heterocyclic ring containing N and a plane of the first and second dibenzoheterocyclic rings is reduced, so that the distribution region of the lowest unoccupied molecular orbital (LUMO) energy level of the organic compound tends to be more planar, thereby further improving the electron transport property and structural stability of the organic compound. Therefore, the luminous efficiency and the service life of the organic electronic device using the organic compound of the embodiments can be improved.

In the disclosure, the terms “first” and “second” are used only for the purpose of description, and cannot be understood as indicating or implying relative importance or implying the number of features indicated. Therefore, the features limited to “first” and “second” may explicitly or implicitly include one or more features. Moreover, the terms “a plurality of” and “multiple” refer to two or more than two, unless otherwise specified.

In the above embodiments, the description of each embodiment has its own emphasis, and for parts not described in detail in a certain embodiment, please refer to relevant description of other embodiments.

The embodiments, examples, and related technical features of the disclosure may be combined and replaced with each other without conflict.

The above are merely preferred embodiments of the disclosure, and do not limit the disclosure in any form. Any simple modifications, equivalent changes, and modifications made to the above embodiments according to the technical essence of the disclosure without departing from the contents of the technical solutions of the disclosure still fall within the scope of the technical solutions of the disclosure.

What is claimed is:

1. An organic compound having a chemical structure represented by the following formula (1): 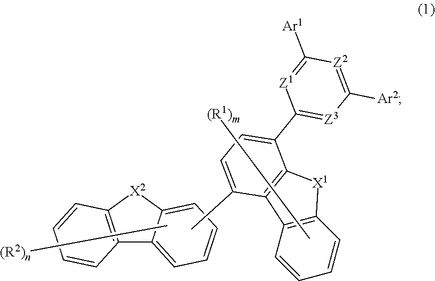
wherein X¹ is selected from O or S;
X² is selected from O, S, or NR³;
Z¹, Z², and Z³ are independently selected from CH or N, and at least one of Z¹, Z², and Z³ is N;
Ar¹ and Ar² are independently selected from a substituted or unsubstituted aromatic group having 6 to 20 carbon atoms, or a substituted or unsubstituted heteroaromatic group having 5 to 20 carbon atoms;
R¹ is selected from a hydrogen atom or a deuterium atom;
R² is selected from a hydrogen atom, a deuterium atom, a substituted or unsubstituted aromatic group having 6 to 20 carbon atoms, or a substituted or unsubstituted heteroaromatic group having 5 to 20 carbon atoms;
R³ is selected from a substituted or unsubstituted aromatic group having 6 to 18 carbon atoms, or a substituted or unsubstituted heteroaromatic group having 5 to 18 carbon atoms;
m is any integer from 1 to 6; and
n is any integer from 1 to 7.

2. The organic compound of claim 1, wherein Z¹, Z², and Z³ are N.

3. The organic compound of claim 1, wherein Ar¹ and Ar² are independently selected from a phenyl group, a deuterated phenyl group, a biphenyl group, a triphenyl group, a naphthyl group, a phenanthryl group, a pyridyl group, a pyrimidinyl group, a pyrazinyl group, a quinolinyl group, an isoquinolinyl group, a phenanthrolinyl group, or any combination thereof.

4. The organic compound of claim 1, wherein R² is selected from a hydrogen atom, a deuterium atom, a phenyl group, a deuterated phenyl group, a biphenyl group, a triphenyl group, a naphthyl group, a phenanthryl group, a pyridyl group, a pyrimidinyl group, a pyrazinyl group, a quinolinyl group, an isoquinolinyl group, a phenanthrolinyl group, or any combination thereof.

5. The organic compound of claim 1, wherein R³ is selected from a phenyl group, a biphenyl group, a deuterated phenyl group, a pyridyl group, or any combination thereof.

6. The organic compound of claim 1, wherein the organic compound has the chemical structure represented by any one of the following formulae (P-1) to (P-160), formulae (Q-1) to (Q-160), formulae (R-1) to (R-128), and formulae (S-1) to (S-100):

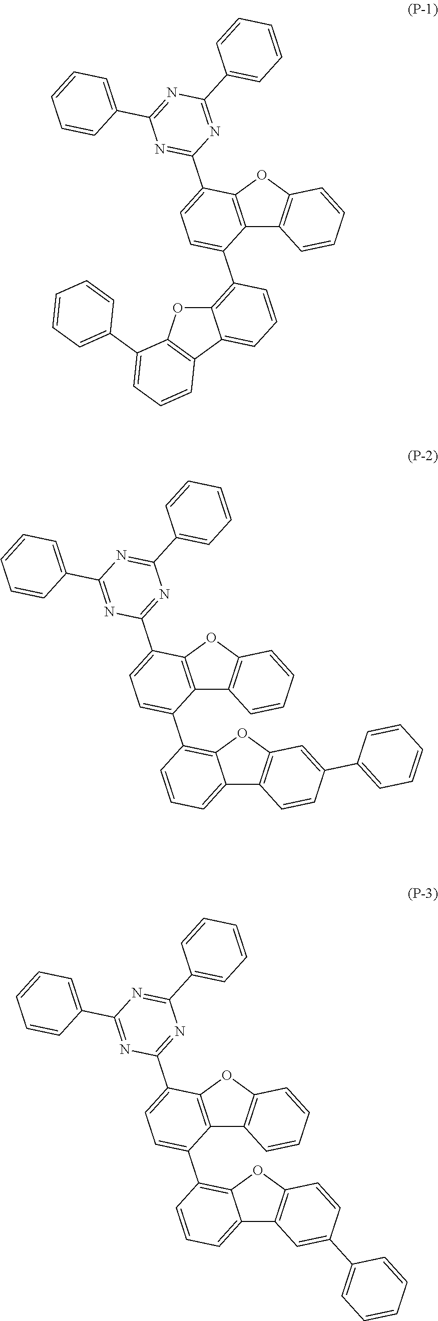 [SMILES: c1ccc(-c2nc(-c3ccccc3)nc(-c3ccc(-c4cccc5c4oc4c(-c6ccccc6)cccc45)c4c3oc3ccccc34)n2)cc1 ; c1ccc(-c2ccc3c(c2)oc2c(-c4ccc(-c5nc(-c6ccccc6)nc(-c6ccccc6)n5)c5oc6ccccc6c45)cccc23)cc1 ; c1ccc(-c2ccc3oc4c(-c5ccc(-c6nc(-c7ccccc7)nc(-c7ccccc7)n6)c6oc7ccccc7c56)cccc4c3c2)cc1]

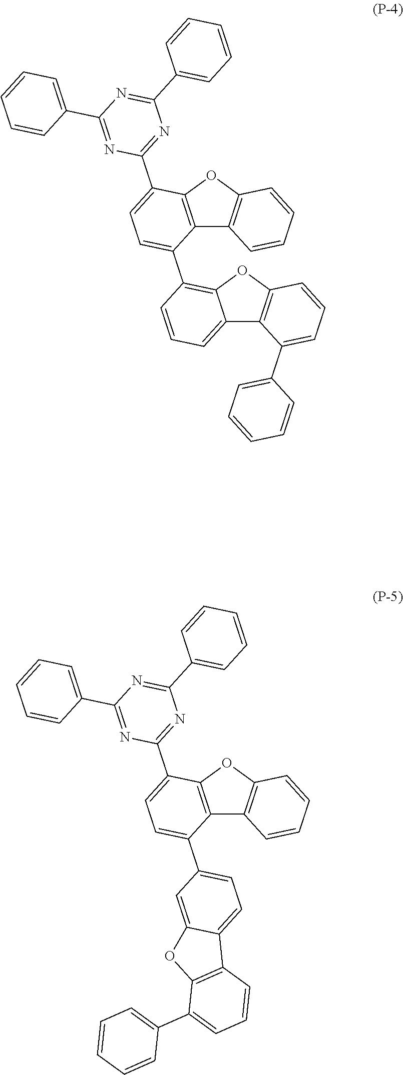 [SMILES: c1ccc(-c2nc(-c3ccccc3)nc(-c3ccc(-c4cccc5c4oc4cccc(-c6ccccc6)c45)c4c3oc3ccccc34)n2)cc1 ; c1ccc(-c2nc(-c3ccccc3)nc(-c3ccc(-c4ccc5c(c4)oc4c(-c6ccccc6)cccc45)c4c3oc3ccccc34)n2)cc1]

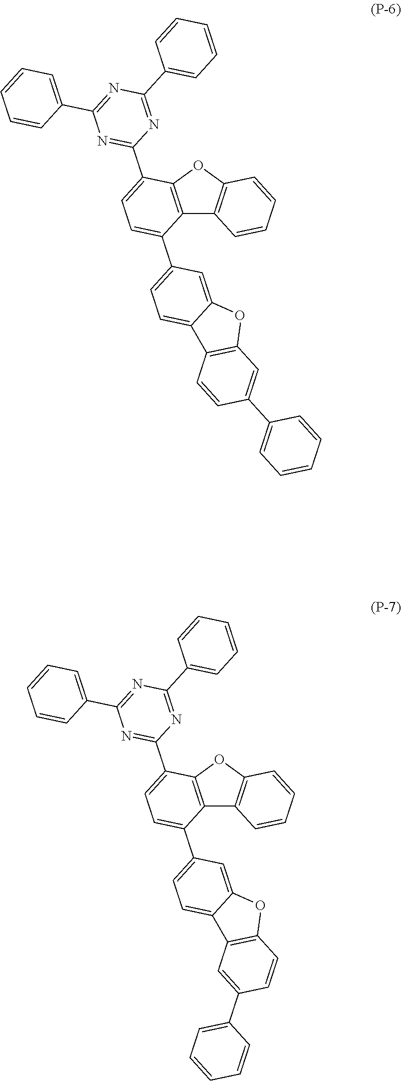 [SMILES: c1ccc(-c2ccc3c(c2)oc2cc(-c4ccc(-c5nc(-c6ccccc6)nc(-c6ccccc6)n5)c5oc6ccccc6c45)ccc23)cc1 ; c1ccc(-c2ccc3oc4cc(-c5ccc(-c6nc(-c7ccccc7)nc(-c7ccccc7)n6)c6oc7ccccc7c56)ccc4c3c2)cc1]

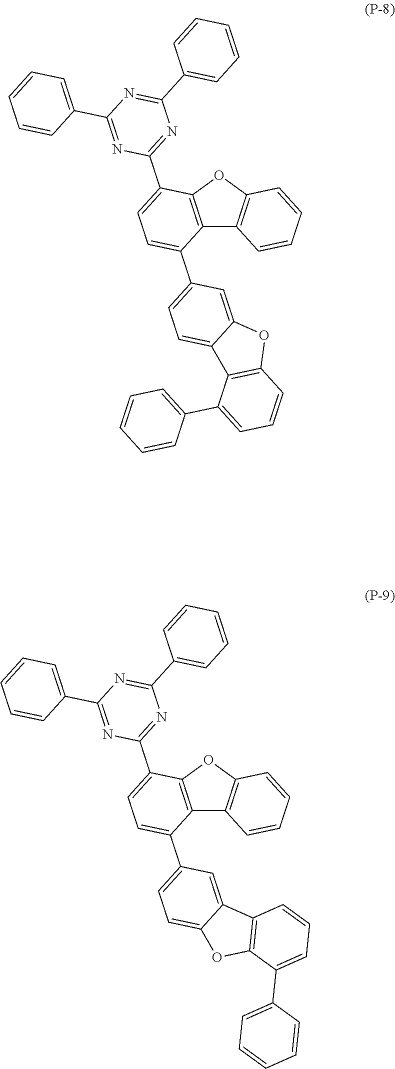 [SMILES: c1ccc(-c2nc(-c3ccccc3)nc(-c3ccc(-c4ccc5c(c4)oc4cccc(-c6ccccc6)c45)c4c3oc3ccccc34)n2)cc1 ; c1ccc(-c2nc(-c3ccccc3)nc(-c3ccc(-c4ccc5oc6c(-c7ccccc7)cccc6c5c4)c4c3oc3ccccc34)n2)cc1]

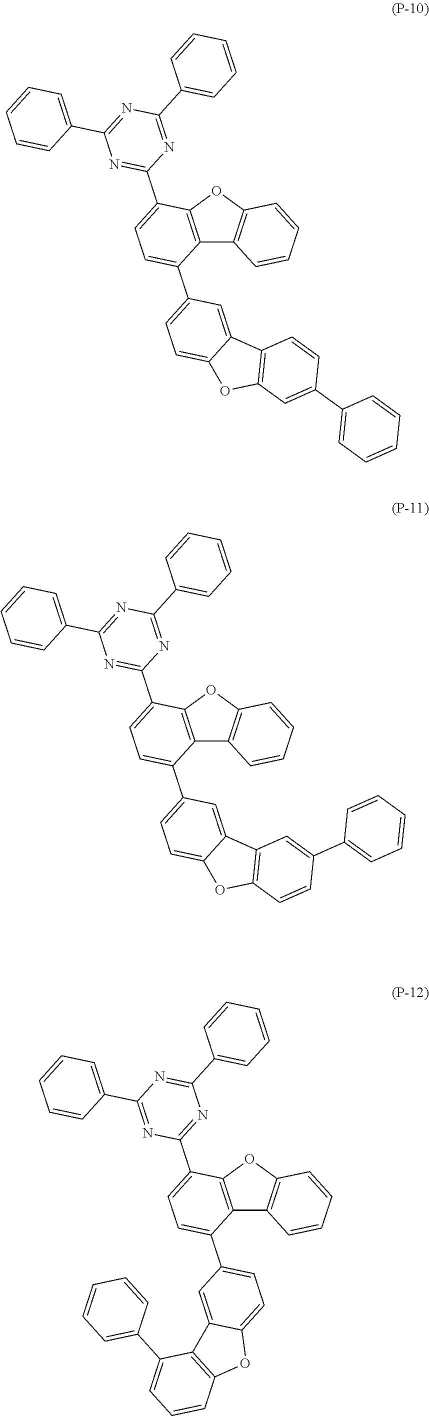 [SMILES: c1ccc(-c2ccc3c(c2)oc2ccc(-c4ccc(-c5nc(-c6ccccc6)nc(-c6ccccc6)n5)c5oc6ccccc6c45)cc23)cc1 ; c1ccc(-c2ccc3oc4ccc(-c5ccc(-c6nc(-c7ccccc7)nc(-c7ccccc7)n6)c6oc7ccccc7c56)cc4c3c2)cc1 ; c1ccc(-c2nc(-c3ccccc3)nc(-c3ccc(-c4ccc5oc6cccc(-c7ccccc7)c6c5c4)c4c3oc3ccccc34)n2)cc1]

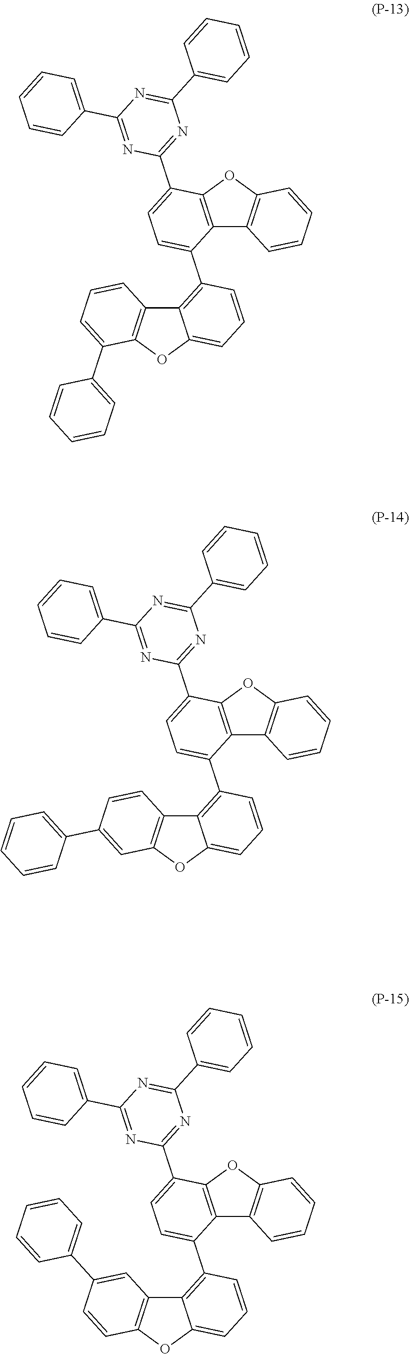 [SMILES: c1ccc(-c2nc(-c3ccccc3)nc(-c3ccc(-c4cccc5oc6c(-c7ccccc7)cccc6c45)c4c3oc3ccccc34)n2)cc1 ; c1ccc(-c2ccc3c(c2)oc2cccc(-c4ccc(-c5nc(-c6ccccc6)nc(-c6ccccc6)n5)c5oc6ccccc6c45)c23)cc1 ; c1ccc(-c2ccc3oc4cccc(-c5ccc(-c6nc(-c7ccccc7)nc(-c7ccccc7)n6)c6oc7ccccc7c56)c4c3c2)cc1]

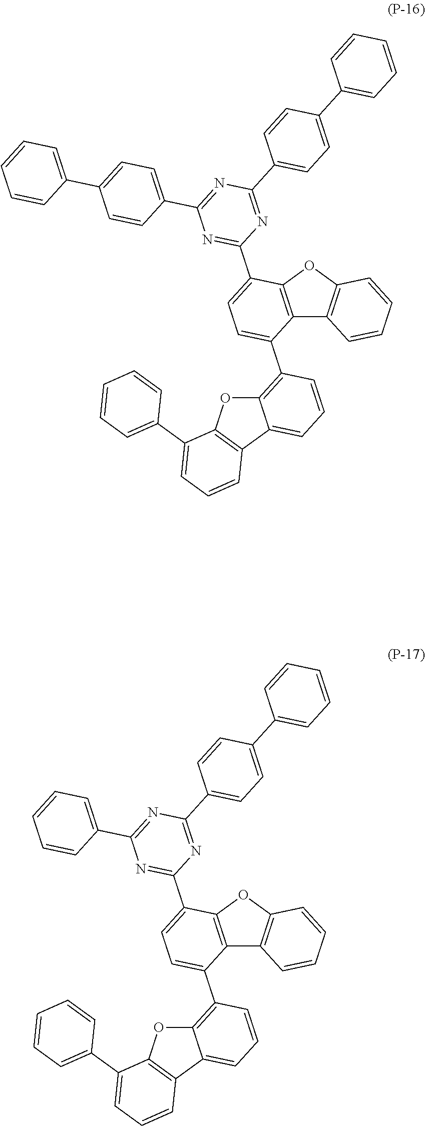 [SMILES: c1ccc(-c2ccc(-c3nc(-c4ccc(-c5ccccc5)cc4)nc(-c4ccc(-c5cccc6c5oc5c(-c7ccccc7)cccc56)c5c4oc4ccccc45)n3)cc2)cc1 ; c1ccc(-c2ccc(-c3nc(-c4ccccc4)nc(-c4ccc(-c5cccc6c5oc5c(-c7ccccc7)cccc56)c5c4oc4ccccc45)n3)cc2)cc1]

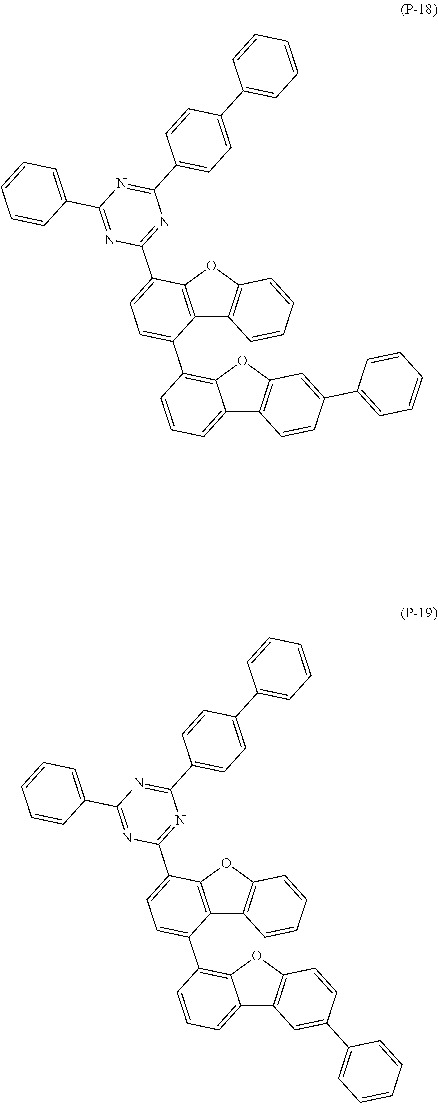 [SMILES: c1ccc(-c2ccc(-c3nc(-c4ccccc4)nc(-c4ccc(-c5cccc6c5oc5cc(-c7ccccc7)ccc56)c5c4oc4ccccc45)n3)cc2)cc1 ; c1ccc(-c2ccc(-c3nc(-c4ccccc4)nc(-c4ccc(-c5cccc6c5oc5ccc(-c7ccccc7)cc56)c5c4oc4ccccc45)n3)cc2)cc1]

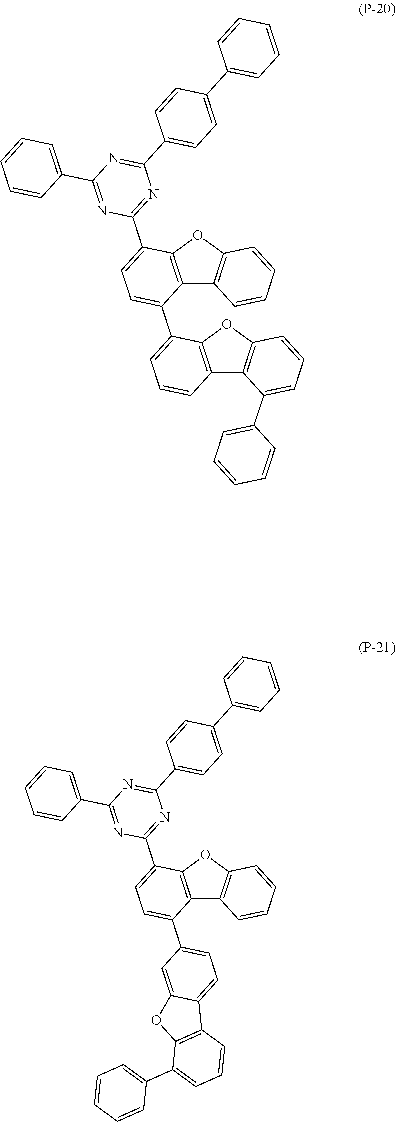 [SMILES: c1ccc(-c2ccc(-c3nc(-c4ccccc4)nc(-c4ccc(-c5cccc6c5oc5cccc(-c7ccccc7)c56)c5c4oc4ccccc45)n3)cc2)cc1 ; c1ccc(-c2ccc(-c3nc(-c4ccccc4)nc(-c4ccc(-c5ccc6c(c5)oc5c(-c7ccccc7)cccc56)c5c4oc4ccccc45)n3)cc2)cc1]

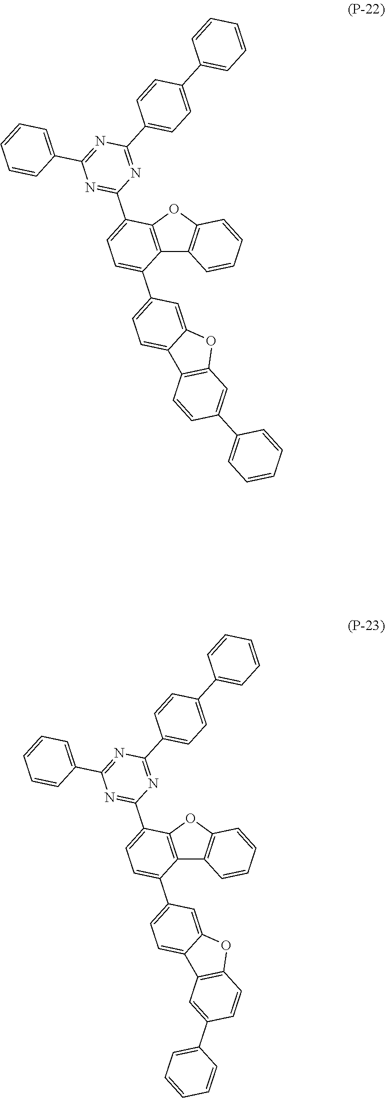 [SMILES: c1ccc(-c2ccc(-c3nc(-c4ccccc4)nc(-c4ccc(-c5ccc6c(c5)oc5cc(-c7ccccc7)ccc56)c5c4oc4ccccc45)n3)cc2)cc1 ; c1ccc(-c2ccc(-c3nc(-c4ccccc4)nc(-c4ccc(-c5ccc6c(c5)oc5ccc(-c7ccccc7)cc56)c5c4oc4ccccc45)n3)cc2)cc1]

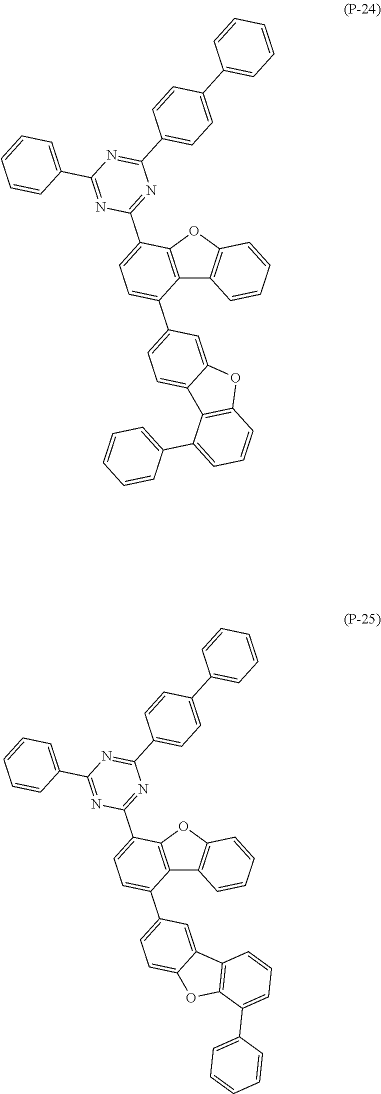 [SMILES: c1ccc(-c2ccc(-c3nc(-c4ccccc4)nc(-c4ccc(-c5ccc6c(c5)oc5cccc(-c7ccccc7)c56)c5c4oc4ccccc45)n3)cc2)cc1 ; c1ccc(-c2ccc(-c3nc(-c4ccccc4)nc(-c4ccc(-c5ccc6oc7c(-c8ccccc8)cccc7c6c5)c5c4oc4ccccc45)n3)cc2)cc1]

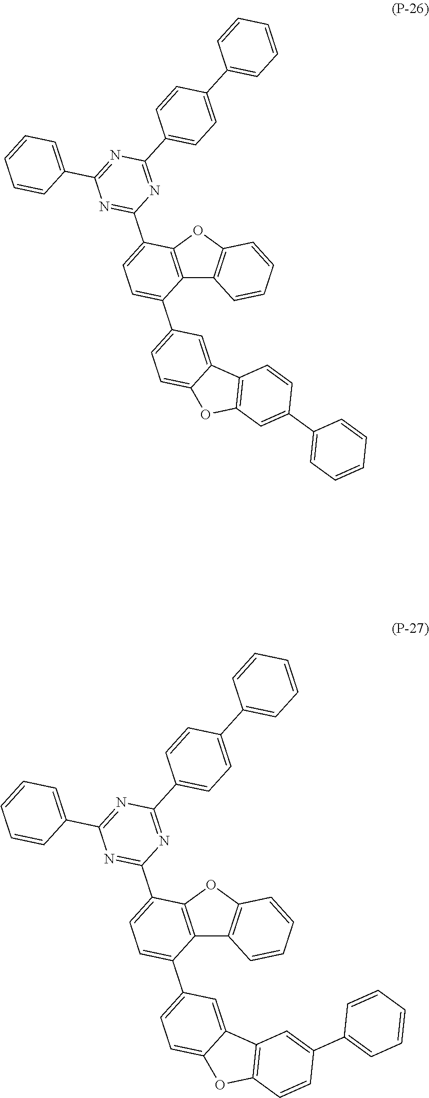 [SMILES: c1ccc(-c2ccc(-c3nc(-c4ccccc4)nc(-c4ccc(-c5ccc6oc7cc(-c8ccccc8)ccc7c6c5)c5c4oc4ccccc45)n3)cc2)cc1 ; c1ccc(-c2ccc(-c3nc(-c4ccccc4)nc(-c4ccc(-c5ccc6oc7ccc(-c8ccccc8)cc7c6c5)c5c4oc4ccccc45)n3)cc2)cc1]

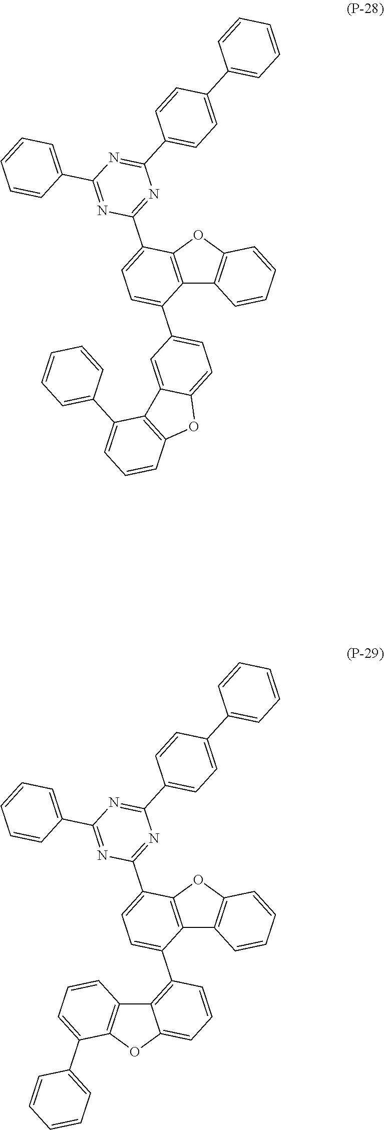 [SMILES: c1ccc(-c2ccc(-c3nc(-c4ccccc4)nc(-c4ccc(-c5ccc6oc7cccc(-c8ccccc8)c7c6c5)c5c4oc4ccccc45)n3)cc2)cc1 ; c1ccc(-c2ccc(-c3nc(-c4ccccc4)nc(-c4ccc(-c5cccc6oc7c(-c8ccccc8)cccc7c56)c5c4oc4ccccc45)n3)cc2)cc1]

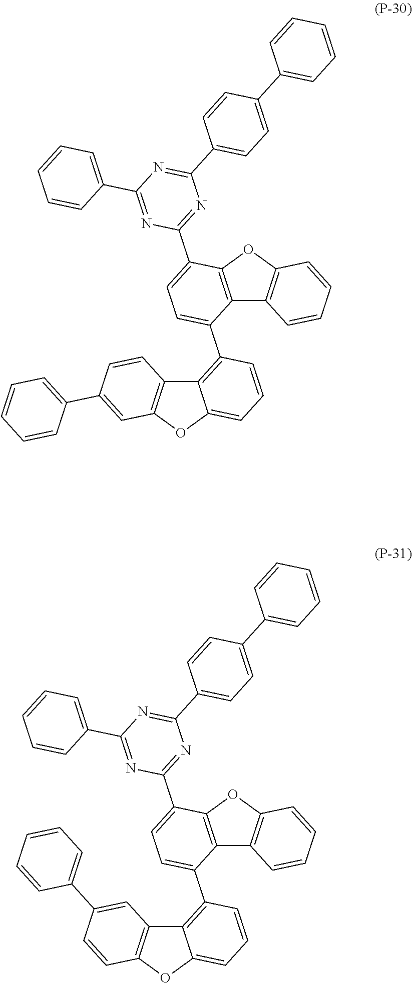 [SMILES: c1ccc(-c2ccc(-c3nc(-c4ccccc4)nc(-c4ccc(-c5cccc6oc7cc(-c8ccccc8)ccc7c56)c5c4oc4ccccc45)n3)cc2)cc1 ; c1ccc(-c2ccc(-c3nc(-c4ccccc4)nc(-c4ccc(-c5cccc6oc7ccc(-c8ccccc8)cc7c56)c5c4oc4ccccc45)n3)cc2)cc1]

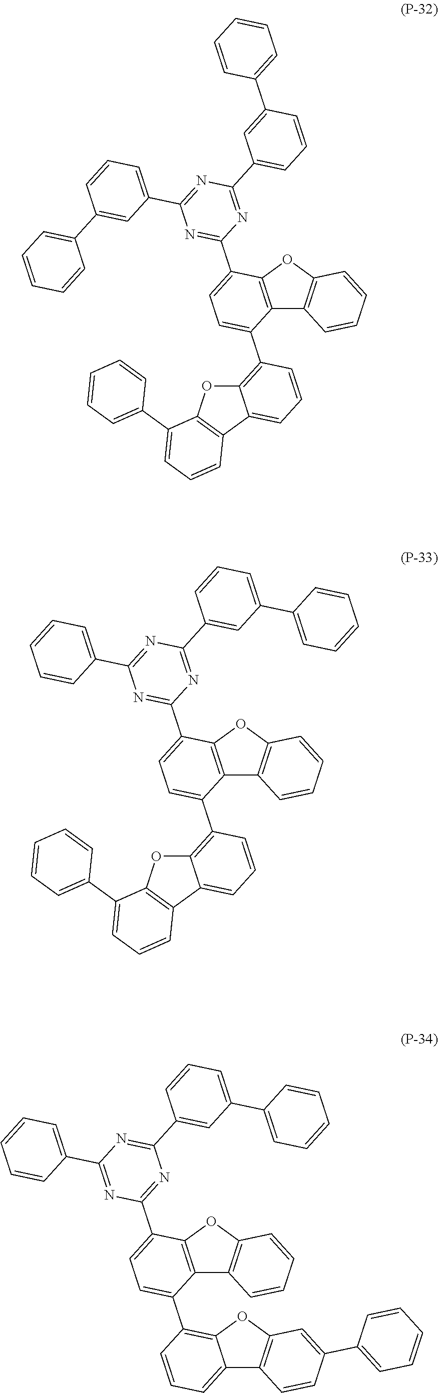 [SMILES: c1ccc(-c2cccc(-c3nc(-c4cccc(-c5ccccc5)c4)nc(-c4ccc(-c5cccc6c5oc5c(-c7ccccc7)cccc56)c5c4oc4ccccc45)n3)c2)cc1 ; c1ccc(-c2cccc(-c3nc(-c4ccccc4)nc(-c4ccc(-c5cccc6c5oc5c(-c7ccccc7)cccc56)c5c4oc4ccccc45)n3)c2)cc1 ; c1ccc(-c2cccc(-c3nc(-c4ccccc4)nc(-c4ccc(-c5cccc6c5oc5cc(-c7ccccc7)ccc56)c5c4oc4ccccc45)n3)c2)cc1]

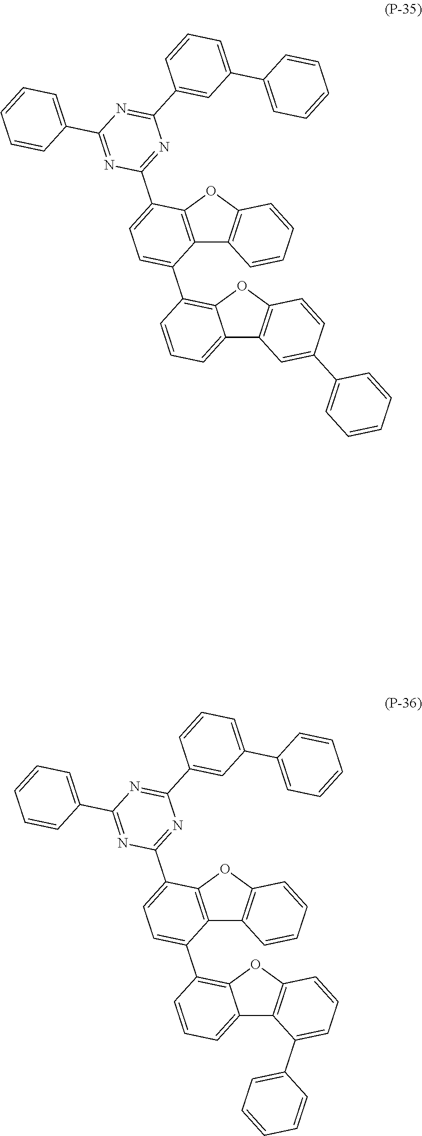 [SMILES: c1ccc(-c2cccc(-c3nc(-c4ccccc4)nc(-c4ccc(-c5cccc6c5oc5ccc(-c7ccccc7)cc56)c5c4oc4ccccc45)n3)c2)cc1 ; c1ccc(-c2cccc(-c3nc(-c4ccccc4)nc(-c4ccc(-c5cccc6c5oc5cccc(-c7ccccc7)c56)c5c4oc4ccccc45)n3)c2)cc1]

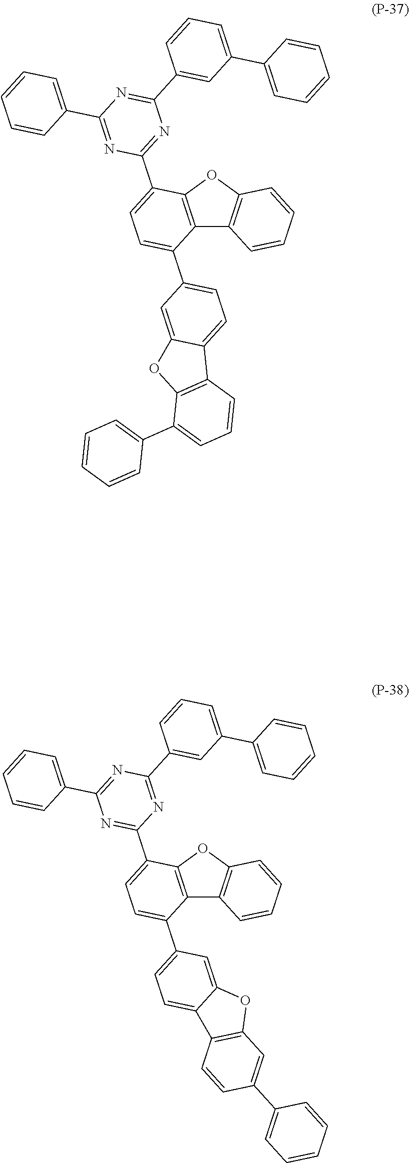 [SMILES: c1ccc(-c2cccc(-c3nc(-c4ccccc4)nc(-c4ccc(-c5ccc6c(c5)oc5c(-c7ccccc7)cccc56)c5c4oc4ccccc45)n3)c2)cc1 ; c1ccc(-c2cccc(-c3nc(-c4ccccc4)nc(-c4ccc(-c5ccc6c(c5)oc5cc(-c7ccccc7)ccc56)c5c4oc4ccccc45)n3)c2)cc1]

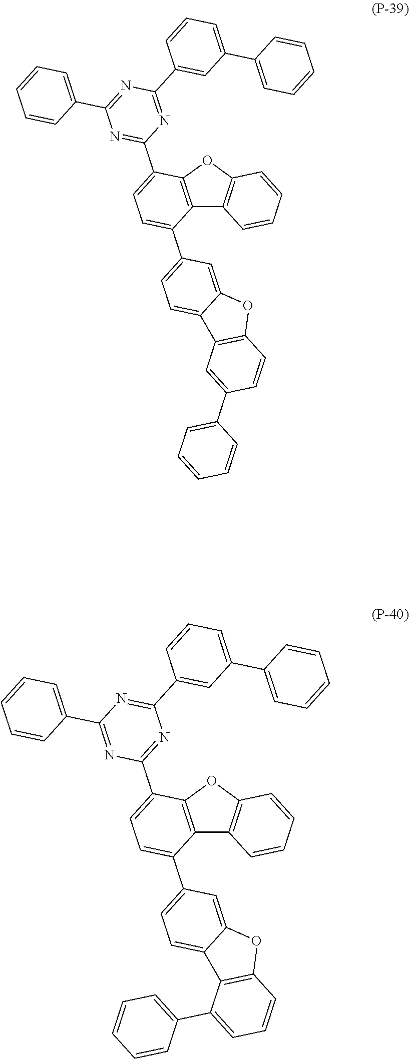 [SMILES: c1ccc(-c2cccc(-c3nc(-c4ccccc4)nc(-c4ccc(-c5ccc6c(c5)oc5ccc(-c7ccccc7)cc56)c5c4oc4ccccc45)n3)c2)cc1 ; c1ccc(-c2cccc(-c3nc(-c4ccccc4)nc(-c4ccc(-c5ccc6c(c5)oc5cccc(-c7ccccc7)c56)c5c4oc4ccccc45)n3)c2)cc1]

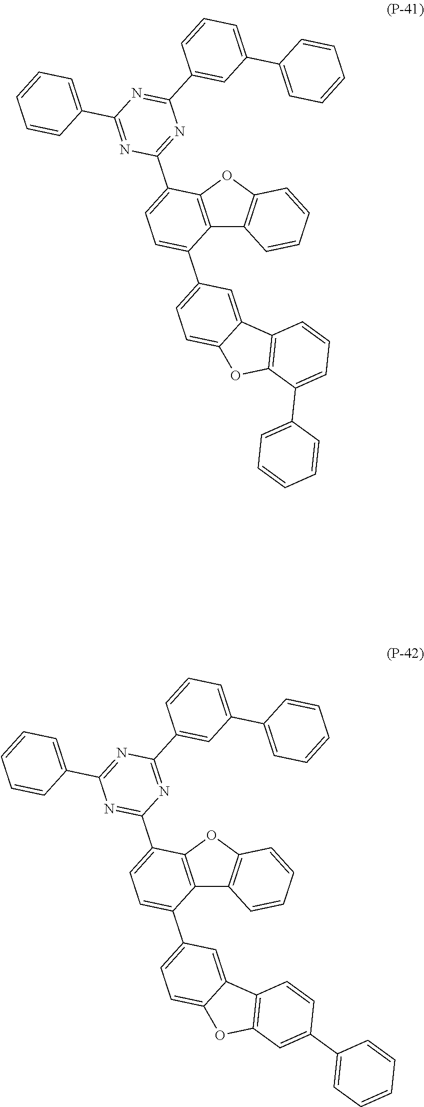 [SMILES: c1ccc(-c2cccc(-c3nc(-c4ccccc4)nc(-c4ccc(-c5ccc6oc7c(-c8ccccc8)cccc7c6c5)c5c4oc4ccccc45)n3)c2)cc1 ; c1ccc(-c2cccc(-c3nc(-c4ccccc4)nc(-c4ccc(-c5ccc6oc7cc(-c8ccccc8)ccc7c6c5)c5c4oc4ccccc45)n3)c2)cc1]

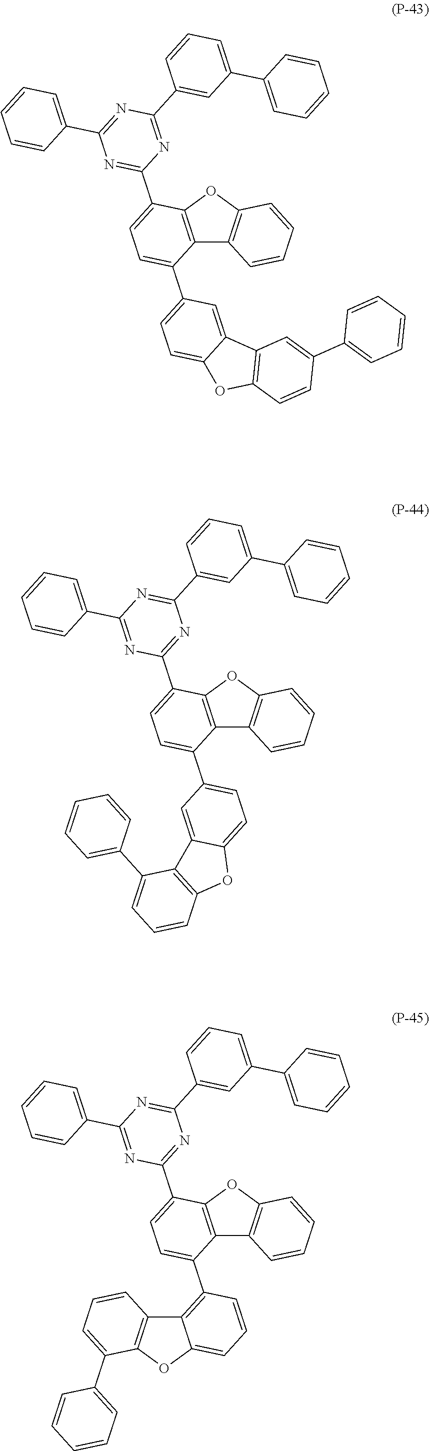 [SMILES: c1ccc(-c2cccc(-c3nc(-c4ccccc4)nc(-c4ccc(-c5ccc6oc7ccc(-c8ccccc8)cc7c6c5)c5c4oc4ccccc45)n3)c2)cc1 ; c1ccc(-c2cccc(-c3nc(-c4ccccc4)nc(-c4ccc(-c5ccc6oc7cccc(-c8ccccc8)c7c6c5)c5c4oc4ccccc45)n3)c2)cc1 ; c1ccc(-c2cccc(-c3nc(-c4ccccc4)nc(-c4ccc(-c5cccc6oc7c(-c8ccccc8)cccc7c56)c5c4oc4ccccc45)n3)c2)cc1]

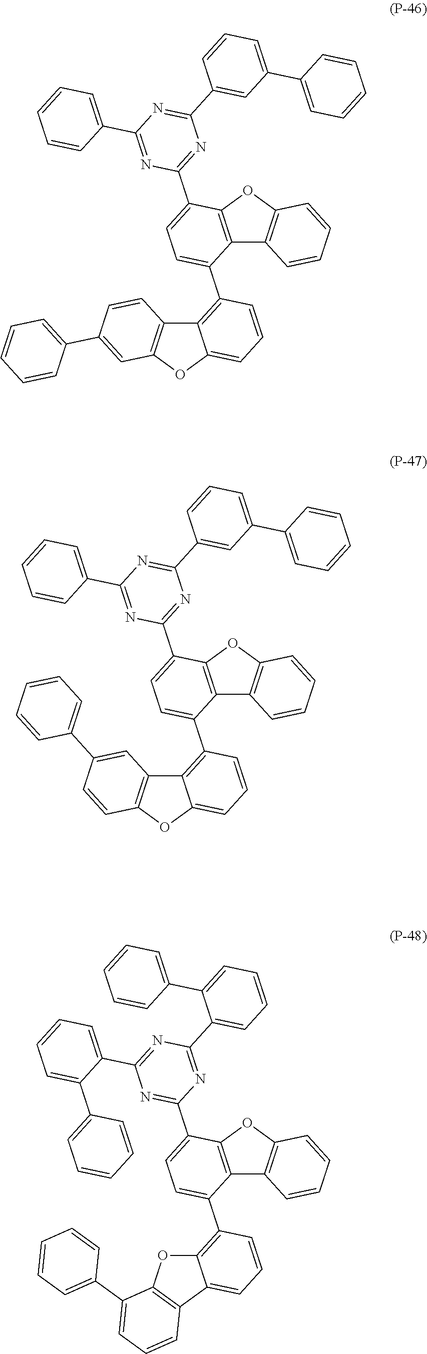 [SMILES: c1ccc(-c2cccc(-c3nc(-c4ccccc4)nc(-c4ccc(-c5cccc6oc7cc(-c8ccccc8)ccc7c56)c5c4oc4ccccc45)n3)c2)cc1 ; c1ccc(-c2cccc(-c3nc(-c4ccccc4)nc(-c4ccc(-c5cccc6oc7ccc(-c8ccccc8)cc7c56)c5c4oc4ccccc45)n3)c2)cc1 ; c1ccc(-c2ccccc2-c2nc(-c3ccccc3-c3ccccc3)nc(-c3ccc(-c4cccc5c4oc4c(-c6ccccc6)cccc45)c4c3oc3ccccc34)n2)cc1]

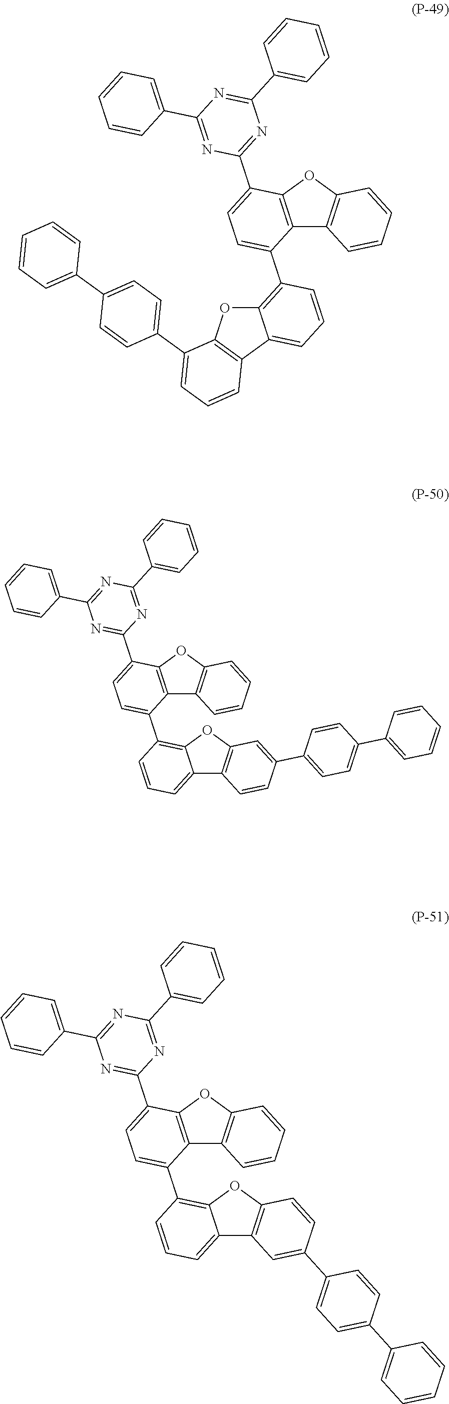 [SMILES: c1ccc(-c2ccc(-c3cccc4c3oc3c(-c5ccc(-c6nc(-c7ccccc7)nc(-c7ccccc7)n6)c6oc7ccccc7c56)cccc34)cc2)cc1 ; c1ccc(-c2ccc(-c3ccc4c(c3)oc3c(-c5ccc(-c6nc(-c7ccccc7)nc(-c7ccccc7)n6)c6oc7ccccc7c56)cccc34)cc2)cc1 ; c1ccc(-c2ccc(-c3ccc4oc5c(-c6ccc(-c7nc(-c8ccccc8)nc(-c8ccccc8)n7)c7oc8ccccc8c67)cccc5c4c3)cc2)cc1]

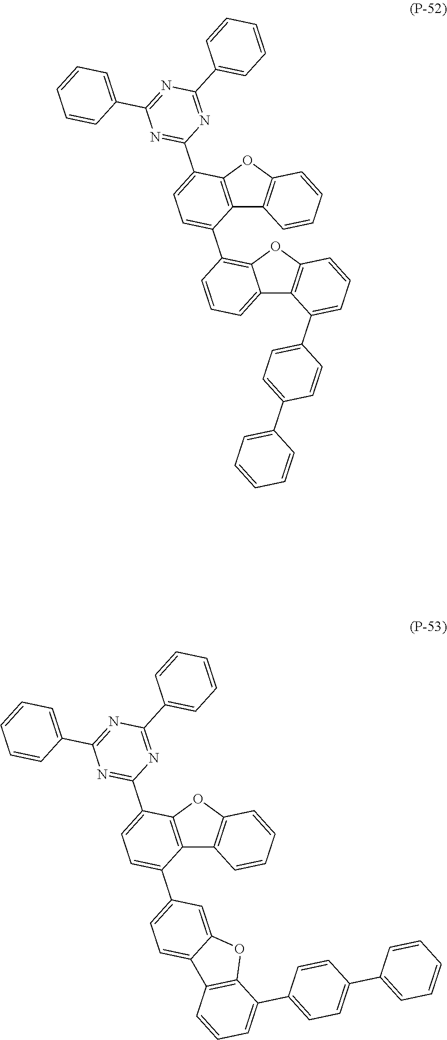 [SMILES: c1ccc(-c2ccc(-c3cccc4oc5c(-c6ccc(-c7nc(-c8ccccc8)nc(-c8ccccc8)n7)c7oc8ccccc8c67)cccc5c34)cc2)cc1 ; c1ccc(-c2ccc(-c3cccc4c3oc3cc(-c5ccc(-c6nc(-c7ccccc7)nc(-c7ccccc7)n6)c6oc7ccccc7c56)ccc34)cc2)cc1]

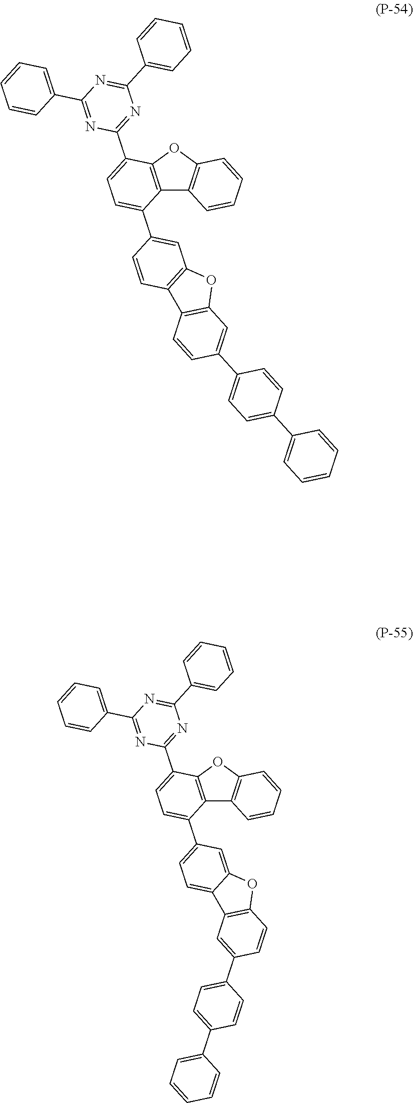 [SMILES: c1ccc(-c2ccc(-c3ccc4c(c3)oc3cc(-c5ccc(-c6nc(-c7ccccc7)nc(-c7ccccc7)n6)c6oc7ccccc7c56)ccc34)cc2)cc1 ; c1ccc(-c2ccc(-c3ccc4oc5cc(-c6ccc(-c7nc(-c8ccccc8)nc(-c8ccccc8)n7)c7oc8ccccc8c67)ccc5c4c3)cc2)cc1]

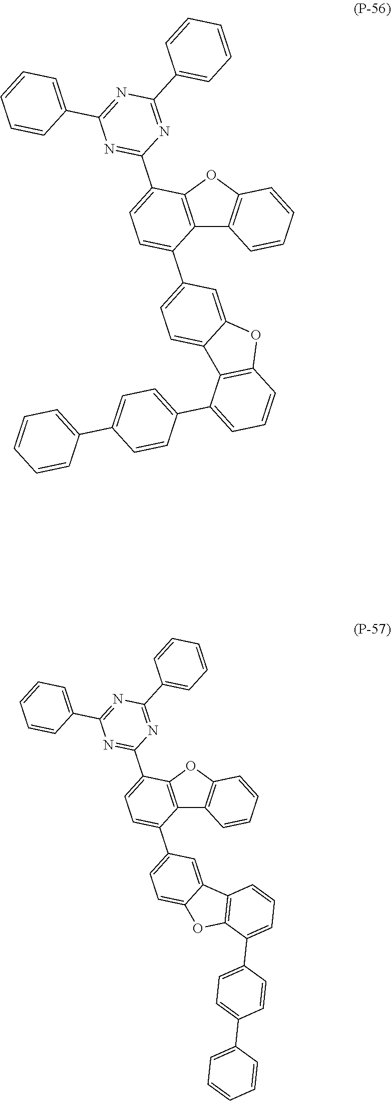 [SMILES: c1ccc(-c2ccc(-c3cccc4oc5cc(-c6ccc(-c7nc(-c8ccccc8)nc(-c8ccccc8)n7)c7oc8ccccc8c67)ccc5c34)cc2)cc1 ; c1ccc(-c2ccc(-c3cccc4c3oc3ccc(-c5ccc(-c6nc(-c7ccccc7)nc(-c7ccccc7)n6)c6oc7ccccc7c56)cc34)cc2)cc1]

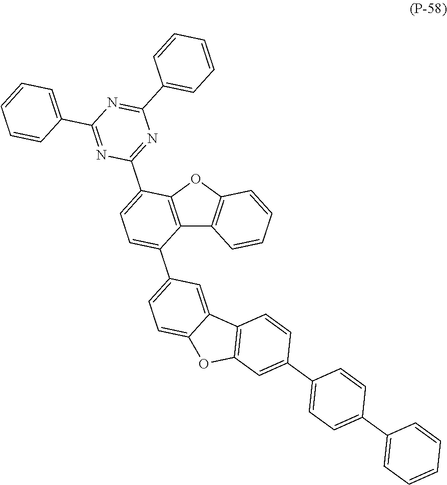 [SMILES: c1ccc(-c2ccc(-c3ccc4c(c3)oc3ccc(-c5ccc(-c6nc(-c7ccccc7)nc(-c7ccccc7)n6)c6oc7ccccc7c56)cc34)cc2)cc1]

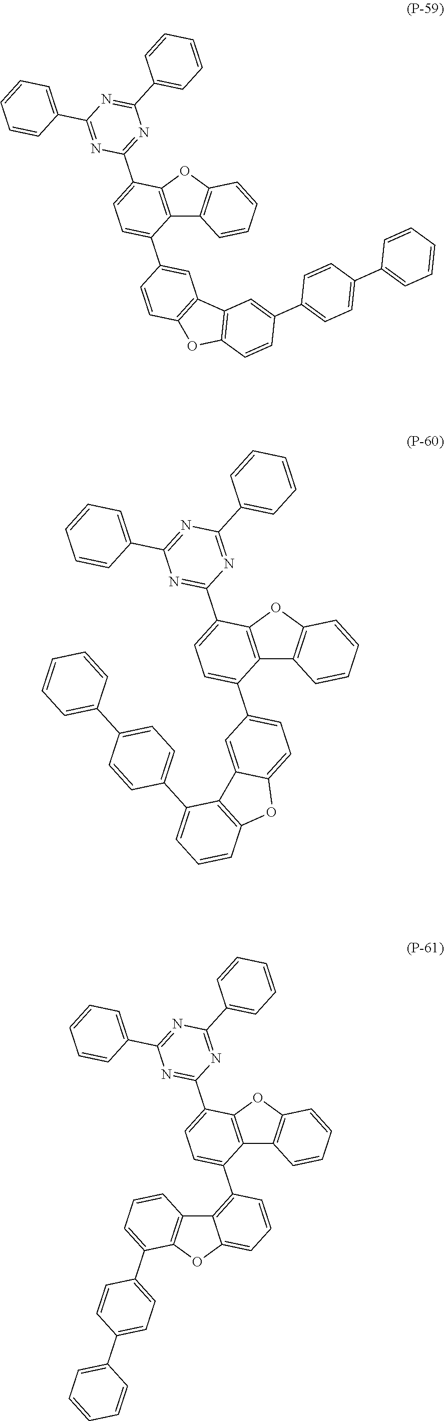 [SMILES: c1ccc(-c2ccc(-c3ccc4oc5ccc(-c6ccc(-c7nc(-c8ccccc8)nc(-c8ccccc8)n7)c7oc8ccccc8c67)cc5c4c3)cc2)cc1 ; c1ccc(-c2ccc(-c3cccc4oc5ccc(-c6ccc(-c7nc(-c8ccccc8)nc(-c8ccccc8)n7)c7oc8ccccc8c67)cc5c34)cc2)cc1 ; c1ccc(-c2ccc(-c3cccc4c3oc3cccc(-c5ccc(-c6nc(-c7ccccc7)nc(-c7ccccc7)n6)c6oc7ccccc7c56)c34)cc2)cc1]

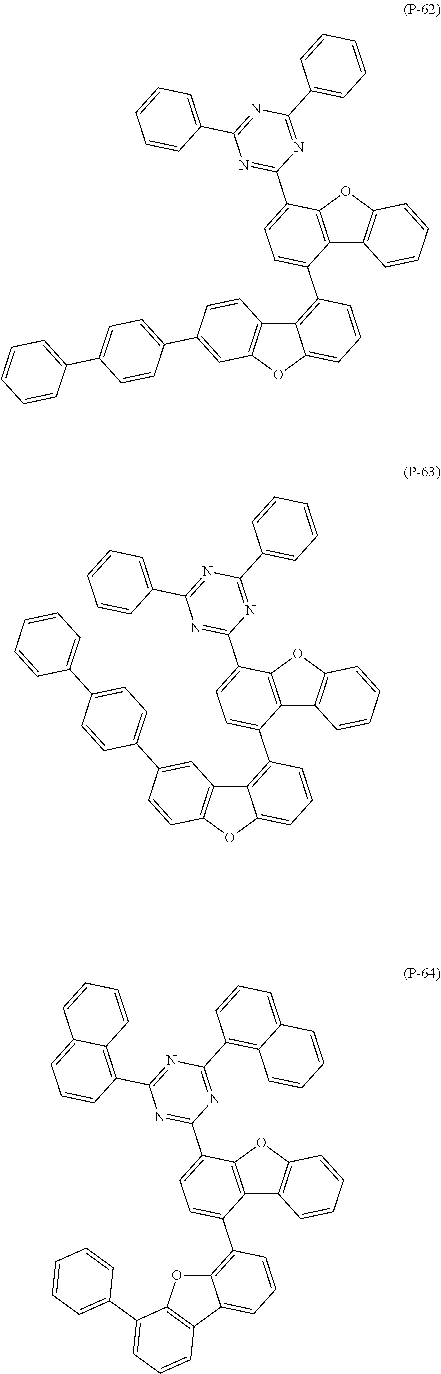 [SMILES: c1ccc(-c2ccc(-c3ccc4c(c3)oc3cccc(-c5ccc(-c6nc(-c7ccccc7)nc(-c7ccccc7)n6)c6oc7ccccc7c56)c34)cc2)cc1 ; c1ccc(-c2ccc(-c3ccc4oc5cccc(-c6ccc(-c7nc(-c8ccccc8)nc(-c8ccccc8)n7)c7oc8ccccc8c67)c5c4c3)cc2)cc1 ; c1ccc(-c2cccc3c2oc2c(-c4ccc(-c5nc(-c6cccc7ccccc67)nc(-c6cccc7ccccc67)n5)c5oc6ccccc6c45)cccc23)cc1]

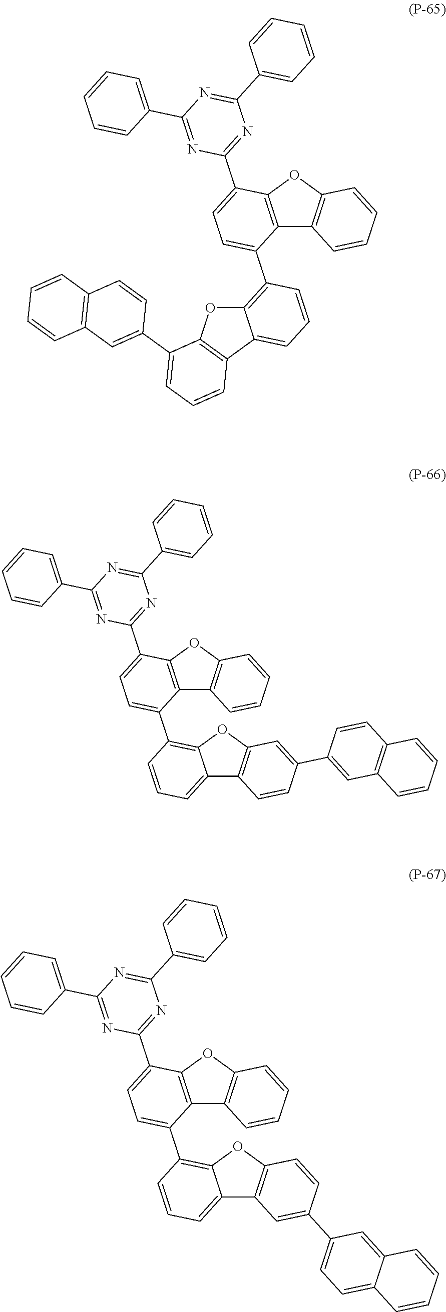 [SMILES: c1ccc(-c2nc(-c3ccccc3)nc(-c3ccc(-c4cccc5c4oc4c(-c6ccc7ccccc7c6)cccc45)c4c3oc3ccccc34)n2)cc1 ; c1ccc(-c2nc(-c3ccccc3)nc(-c3ccc(-c4cccc5c4oc4cc(-c6ccc7ccccc7c6)ccc45)c4c3oc3ccccc34)n2)cc1 ; c1ccc(-c2nc(-c3ccccc3)nc(-c3ccc(-c4cccc5c4oc4ccc(-c6ccc7ccccc7c6)cc45)c4c3oc3ccccc34)n2)cc1]

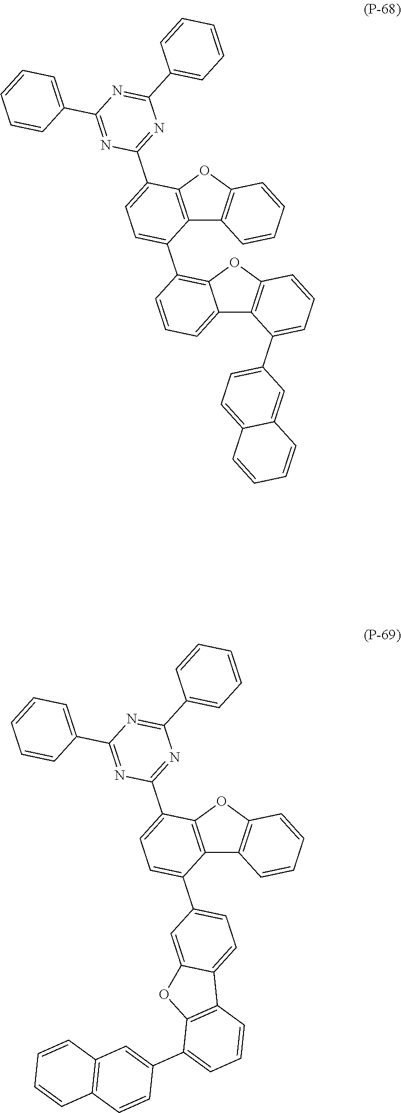 [SMILES: c1ccc(-c2nc(-c3ccccc3)nc(-c3ccc(-c4cccc5c4oc4cccc(-c6ccc7ccccc7c6)c45)c4c3oc3ccccc34)n2)cc1 ; c1ccc(-c2nc(-c3ccccc3)nc(-c3ccc(-c4ccc5c(c4)oc4c(-c6ccc7ccccc7c6)cccc45)c4c3oc3ccccc34)n2)cc1]

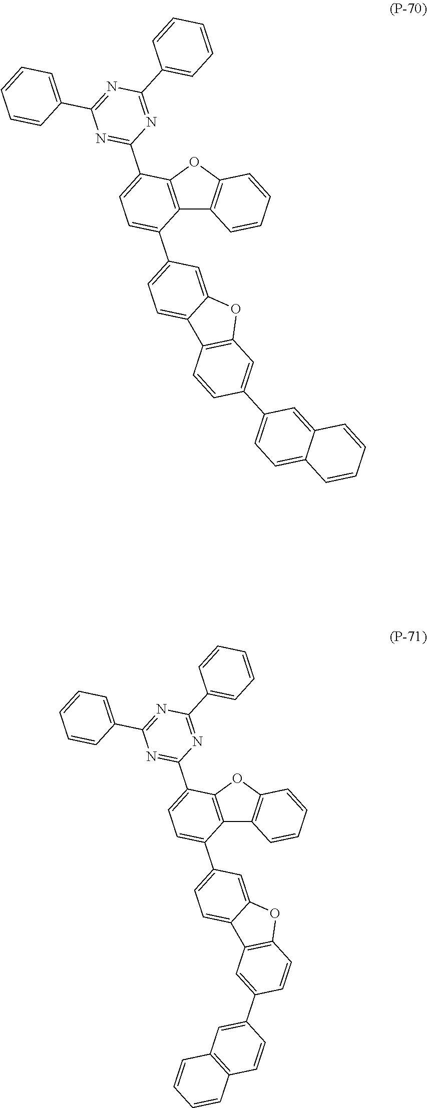 [SMILES: c1ccc(-c2nc(-c3ccccc3)nc(-c3ccc(-c4ccc5c(c4)oc4cc(-c6ccc7ccccc7c6)ccc45)c4c3oc3ccccc34)n2)cc1 ; c1ccc(-c2nc(-c3ccccc3)nc(-c3ccc(-c4ccc5c(c4)oc4ccc(-c6ccc7ccccc7c6)cc45)c4c3oc3ccccc34)n2)cc1]

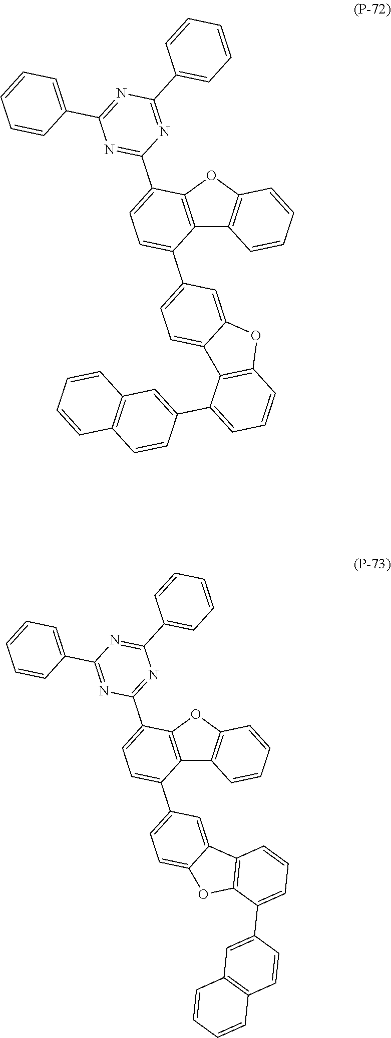 [SMILES: c1ccc(-c2nc(-c3ccccc3)nc(-c3ccc(-c4ccc5c(c4)oc4cccc(-c6ccc7ccccc7c6)c45)c4c3oc3ccccc34)n2)cc1 ; c1ccc(-c2nc(-c3ccccc3)nc(-c3ccc(-c4ccc5oc6c(-c7ccc8ccccc8c7)cccc6c5c4)c4c3oc3ccccc34)n2)cc1]

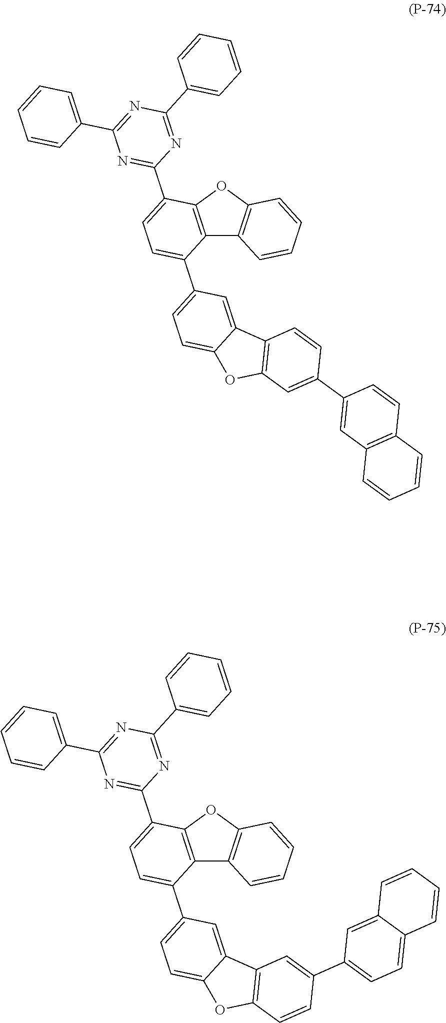 [SMILES: c1ccc(-c2nc(-c3ccccc3)nc(-c3ccc(-c4ccc5oc6cc(-c7ccc8ccccc8c7)ccc6c5c4)c4c3oc3ccccc34)n2)cc1 ; c1ccc(-c2nc(-c3ccccc3)nc(-c3ccc(-c4ccc5oc6ccc(-c7ccc8ccccc8c7)cc6c5c4)c4c3oc3ccccc34)n2)cc1]

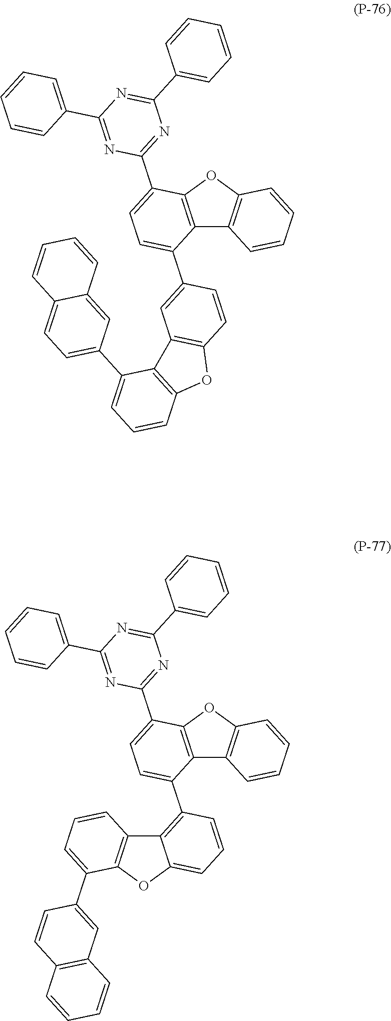 [SMILES: c1ccc(-c2nc(-c3ccccc3)nc(-c3ccc(-c4ccc5oc6cccc(-c7ccc8ccccc8c7)c6c5c4)c4c3oc3ccccc34)n2)cc1 ; c1ccc(-c2nc(-c3ccccc3)nc(-c3ccc(-c4cccc5oc6c(-c7ccc8ccccc8c7)cccc6c45)c4c3oc3ccccc34)n2)cc1]

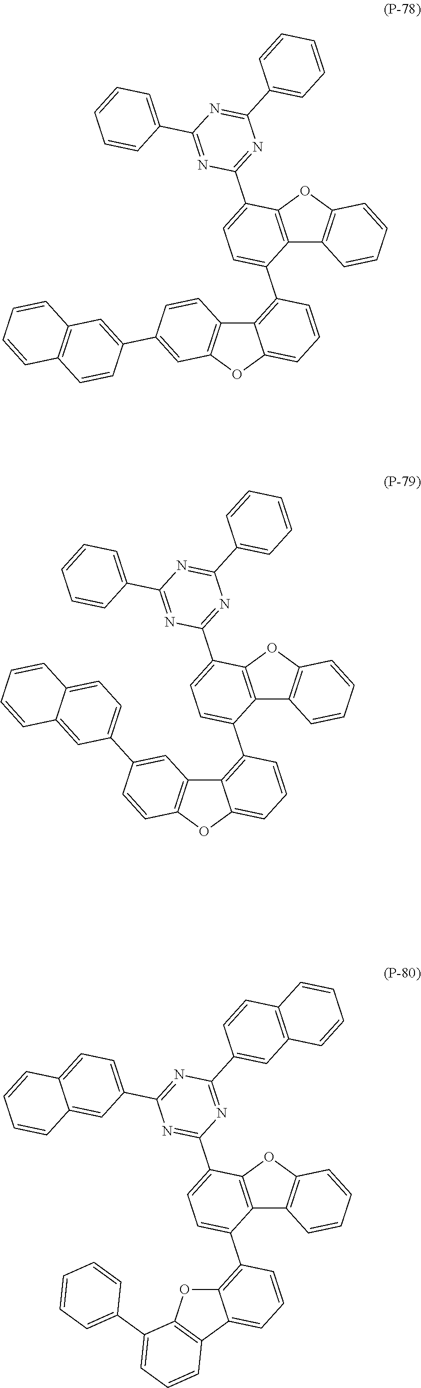 [SMILES: c1ccc(-c2nc(-c3ccccc3)nc(-c3ccc(-c4cccc5oc6cc(-c7ccc8ccccc8c7)ccc6c45)c4c3oc3ccccc34)n2)cc1 ; c1ccc(-c2nc(-c3ccccc3)nc(-c3ccc(-c4cccc5oc6ccc(-c7ccc8ccccc8c7)cc6c45)c4c3oc3ccccc34)n2)cc1 ; c1ccc(-c2cccc3c2oc2c(-c4ccc(-c5nc(-c6ccc7ccccc7c6)nc(-c6ccc7ccccc7c6)n5)c5oc6ccccc6c45)cccc23)cc1]

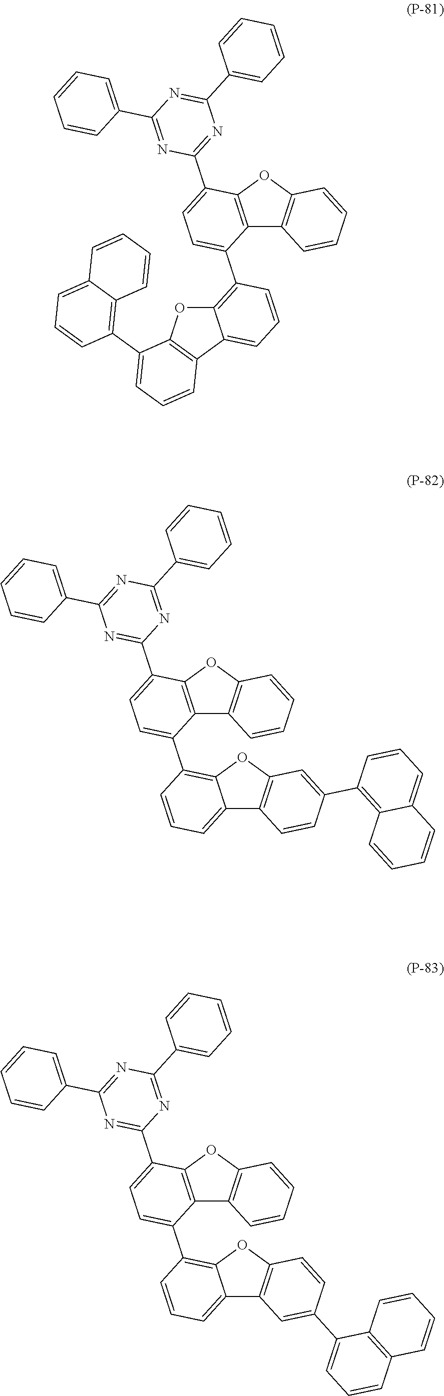 [SMILES: c1ccc(-c2nc(-c3ccccc3)nc(-c3ccc(-c4cccc5c4oc4c(-c6cccc7ccccc67)cccc45)c4c3oc3ccccc34)n2)cc1 ; c1ccc(-c2nc(-c3ccccc3)nc(-c3ccc(-c4cccc5c4oc4cc(-c6cccc7ccccc67)ccc45)c4c3oc3ccccc34)n2)cc1 ; c1ccc(-c2nc(-c3ccccc3)nc(-c3ccc(-c4cccc5c4oc4ccc(-c6cccc7ccccc67)cc45)c4c3oc3ccccc34)n2)cc1]

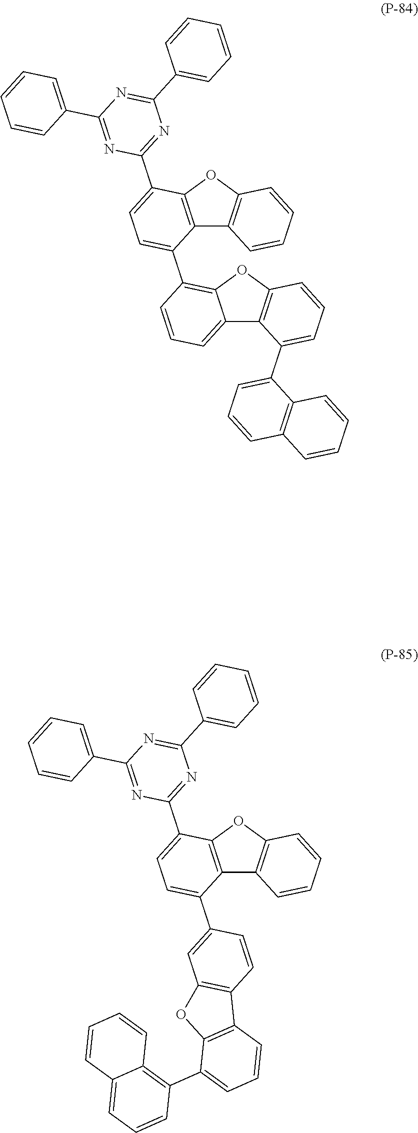 [SMILES: c1ccc(-c2nc(-c3ccccc3)nc(-c3ccc(-c4cccc5c4oc4cccc(-c6cccc7ccccc67)c45)c4c3oc3ccccc34)n2)cc1 ; c1ccc(-c2nc(-c3ccccc3)nc(-c3ccc(-c4ccc5c(c4)oc4c(-c6cccc7ccccc67)cccc45)c4c3oc3ccccc34)n2)cc1]

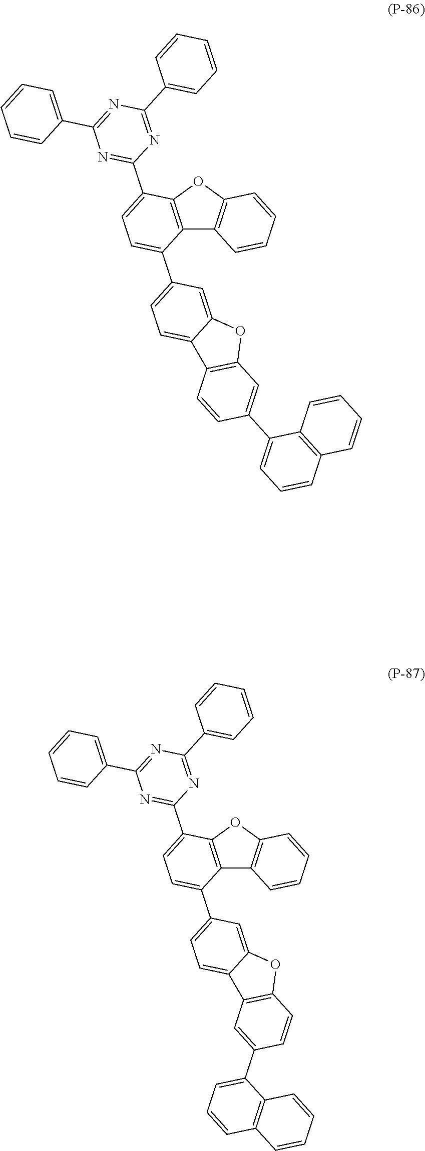 [SMILES: c1ccc(-c2nc(-c3ccccc3)nc(-c3ccc(-c4ccc5c(c4)oc4cc(-c6cccc7ccccc67)ccc45)c4c3oc3ccccc34)n2)cc1 ; c1ccc(-c2nc(-c3ccccc3)nc(-c3ccc(-c4ccc5c(c4)oc4ccc(-c6cccc7ccccc67)cc45)c4c3oc3ccccc34)n2)cc1]

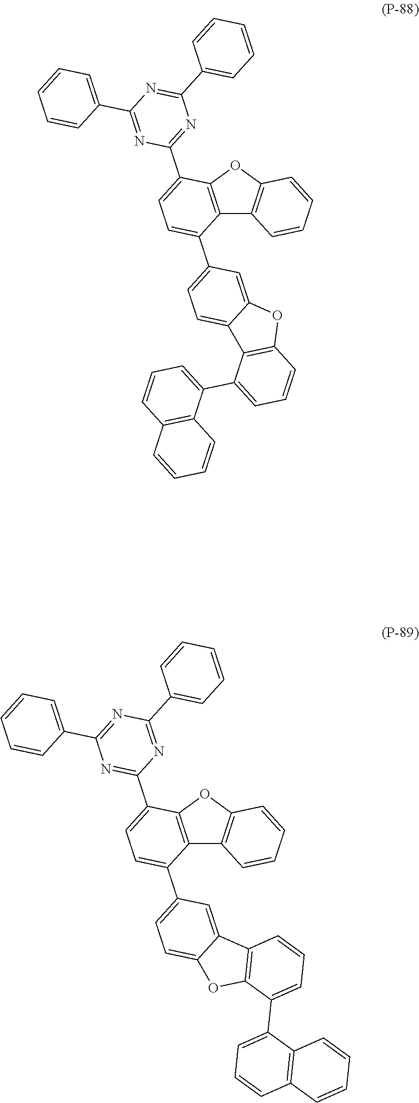 [SMILES: c1ccc(-c2nc(-c3ccccc3)nc(-c3ccc(-c4ccc5c(c4)oc4cccc(-c6cccc7ccccc67)c45)c4c3oc3ccccc34)n2)cc1 ; c1ccc(-c2nc(-c3ccccc3)nc(-c3ccc(-c4ccc5oc6c(-c7cccc8ccccc78)cccc6c5c4)c4c3oc3ccccc34)n2)cc1]

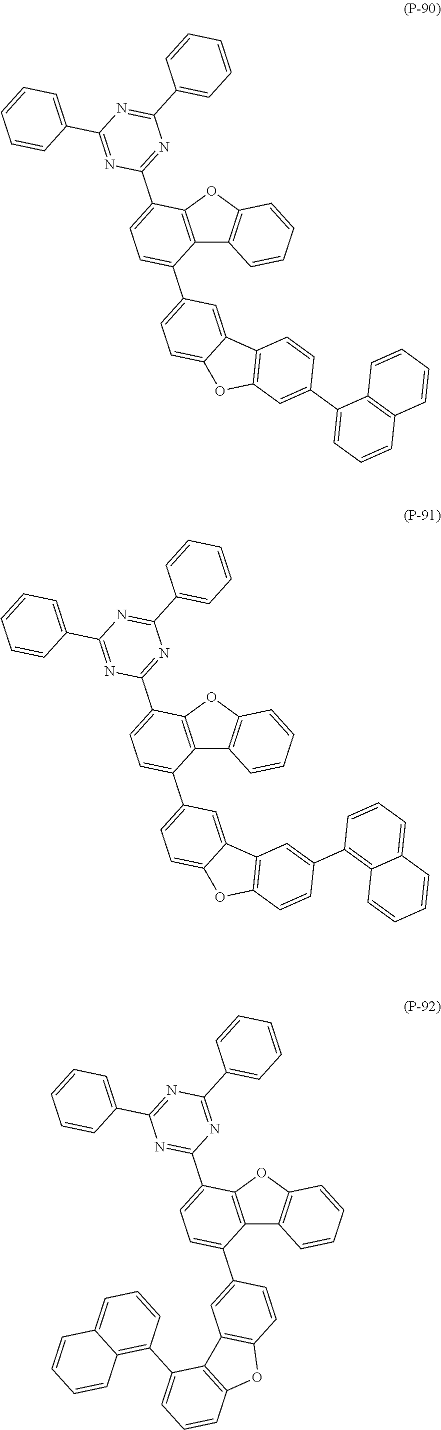 [SMILES: c1ccc(-c2nc(-c3ccccc3)nc(-c3ccc(-c4ccc5oc6cc(-c7cccc8ccccc78)ccc6c5c4)c4c3oc3ccccc34)n2)cc1 ; c1ccc(-c2nc(-c3ccccc3)nc(-c3ccc(-c4ccc5oc6ccc(-c7cccc8ccccc78)cc6c5c4)c4c3oc3ccccc34)n2)cc1 ; c1ccc(-c2nc(-c3ccccc3)nc(-c3ccc(-c4ccc5oc6cccc(-c7cccc8ccccc78)c6c5c4)c4c3oc3ccccc34)n2)cc1]

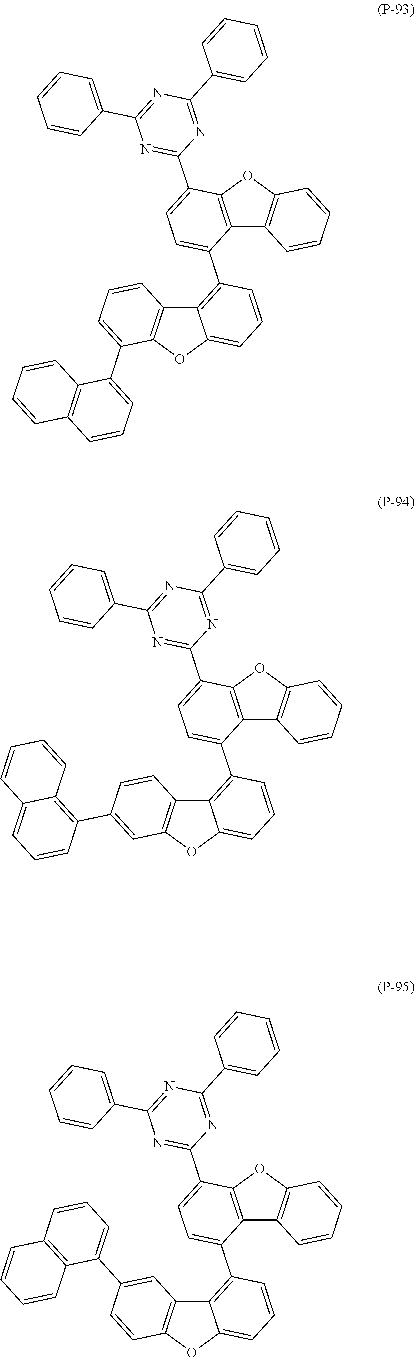 [SMILES: c1ccc(-c2nc(-c3ccccc3)nc(-c3ccc(-c4cccc5oc6c(-c7cccc8ccccc78)cccc6c45)c4c3oc3ccccc34)n2)cc1 ; c1ccc(-c2nc(-c3ccccc3)nc(-c3ccc(-c4cccc5oc6cc(-c7cccc8ccccc78)ccc6c45)c4c3oc3ccccc34)n2)cc1 ; c1ccc(-c2nc(-c3ccccc3)nc(-c3ccc(-c4cccc5oc6ccc(-c7cccc8ccccc78)cc6c45)c4c3oc3ccccc34)n2)cc1]

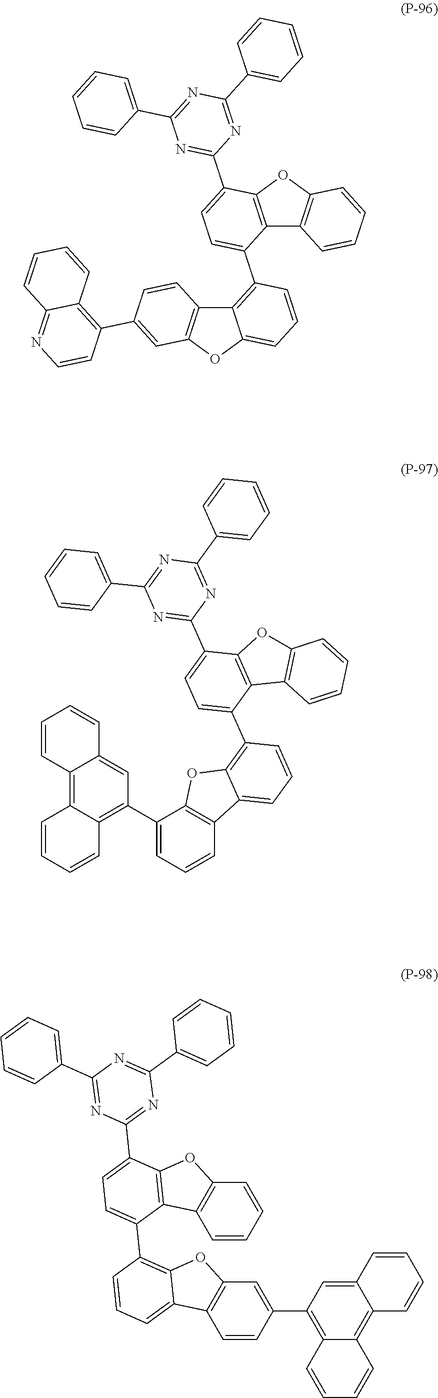 [SMILES: c1ccc(-c2nc(-c3ccccc3)nc(-c3ccc(-c4cccc5oc6cc(-c7ccnc8ccccc78)ccc6c45)c4c3oc3ccccc34)n2)cc1 ; c1ccc(-c2nc(-c3ccccc3)nc(-c3ccc(-c4cccc5c4oc4c(-c6cc7ccccc7c7ccccc67)cccc45)c4c3oc3ccccc34)n2)cc1 ; c1ccc(-c2nc(-c3ccccc3)nc(-c3ccc(-c4cccc5c4oc4cc(-c6cc7ccccc7c7ccccc67)ccc45)c4c3oc3ccccc34)n2)cc1]

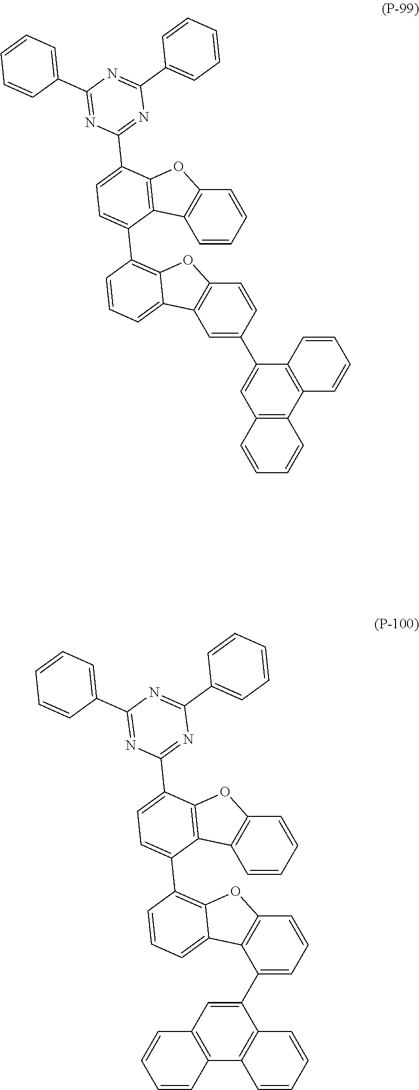 [SMILES: c1ccc(-c2nc(-c3ccccc3)nc(-c3ccc(-c4cccc5c4oc4ccc(-c6cc7ccccc7c7ccccc67)cc45)c4c3oc3ccccc34)n2)cc1 ; c1ccc(-c2nc(-c3ccccc3)nc(-c3ccc(-c4cccc5c4oc4cccc(-c6cc7ccccc7c7ccccc67)c45)c4c3oc3ccccc34)n2)cc1]

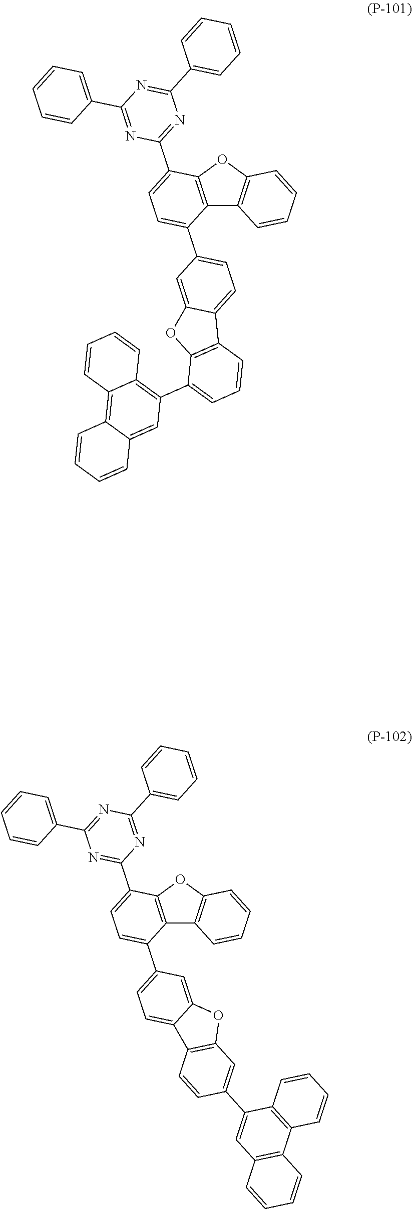 [SMILES: c1ccc(-c2nc(-c3ccccc3)nc(-c3ccc(-c4ccc5c(c4)oc4c(-c6cc7ccccc7c7ccccc67)cccc45)c4c3oc3ccccc34)n2)cc1 ; c1ccc(-c2nc(-c3ccccc3)nc(-c3ccc(-c4ccc5c(c4)oc4cc(-c6cc7ccccc7c7ccccc67)ccc45)c4c3oc3ccccc34)n2)cc1]

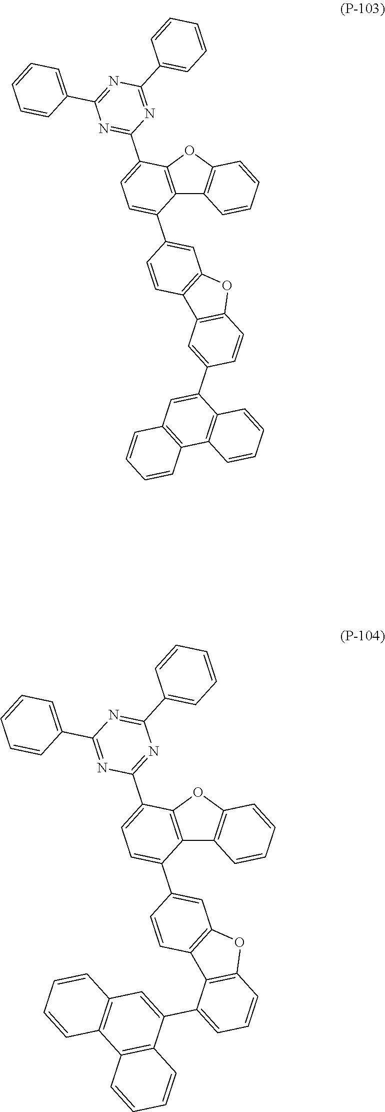 [SMILES: c1ccc(-c2nc(-c3ccccc3)nc(-c3ccc(-c4ccc5c(c4)oc4ccc(-c6cc7ccccc7c7ccccc67)cc45)c4c3oc3ccccc34)n2)cc1 ; c1ccc(-c2nc(-c3ccccc3)nc(-c3ccc(-c4ccc5c(c4)oc4cccc(-c6cc7ccccc7c7ccccc67)c45)c4c3oc3ccccc34)n2)cc1]

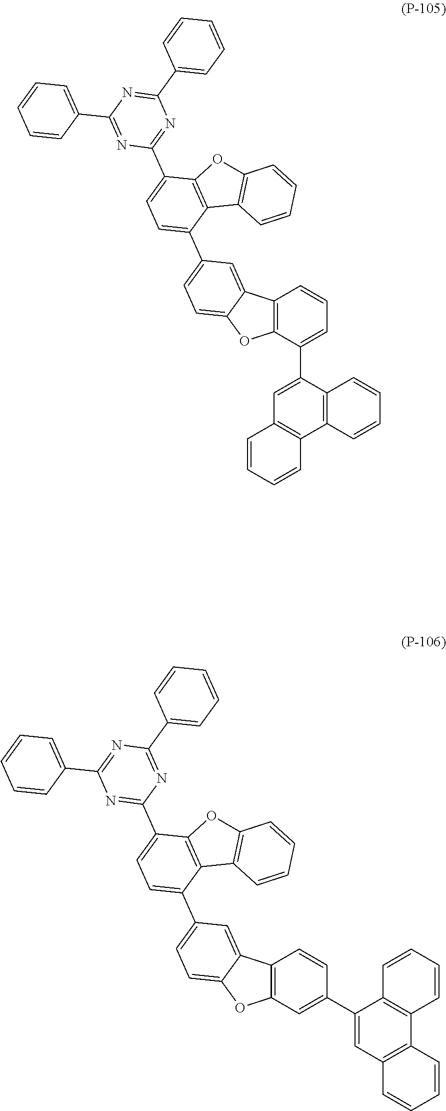 [SMILES: c1ccc(-c2nc(-c3ccccc3)nc(-c3ccc(-c4ccc5oc6c(-c7cc8ccccc8c8ccccc78)cccc6c5c4)c4c3oc3ccccc34)n2)cc1 ; c1ccc(-c2nc(-c3ccccc3)nc(-c3ccc(-c4ccc5oc6cc(-c7cc8ccccc8c8ccccc78)ccc6c5c4)c4c3oc3ccccc34)n2)cc1]

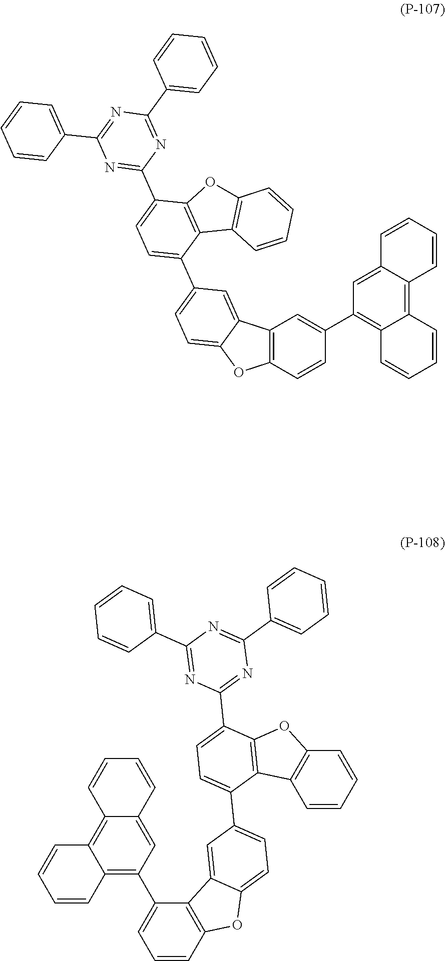 [SMILES: c1ccc(-c2nc(-c3ccccc3)nc(-c3ccc(-c4ccc5oc6ccc(-c7cc8ccccc8c8ccccc78)cc6c5c4)c4c3oc3ccccc34)n2)cc1 ; c1ccc(-c2nc(-c3ccccc3)nc(-c3ccc(-c4ccc5oc6cccc(-c7cc8ccccc8c8ccccc78)c6c5c4)c4c3oc3ccccc34)n2)cc1]

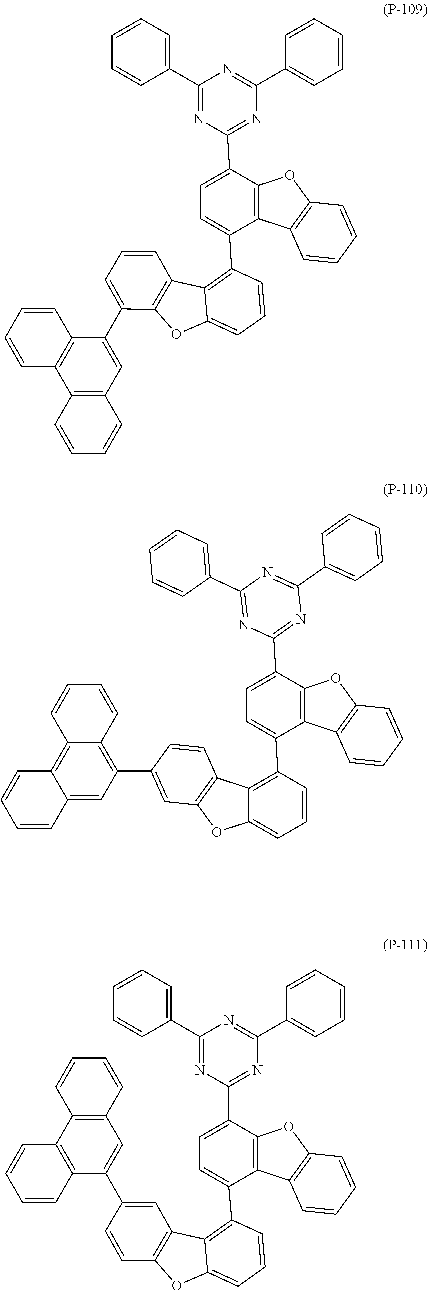 [SMILES: c1ccc(-c2nc(-c3ccccc3)nc(-c3ccc(-c4cccc5oc6c(-c7cc8ccccc8c8ccccc78)cccc6c45)c4c3oc3ccccc34)n2)cc1 ; c1ccc(-c2nc(-c3ccccc3)nc(-c3ccc(-c4cccc5oc6cc(-c7cc8ccccc8c8ccccc78)ccc6c45)c4c3oc3ccccc34)n2)cc1 ; c1ccc(-c2nc(-c3ccccc3)nc(-c3ccc(-c4cccc5oc6ccc(-c7cc8ccccc8c8ccccc78)cc6c45)c4c3oc3ccccc34)n2)cc1]

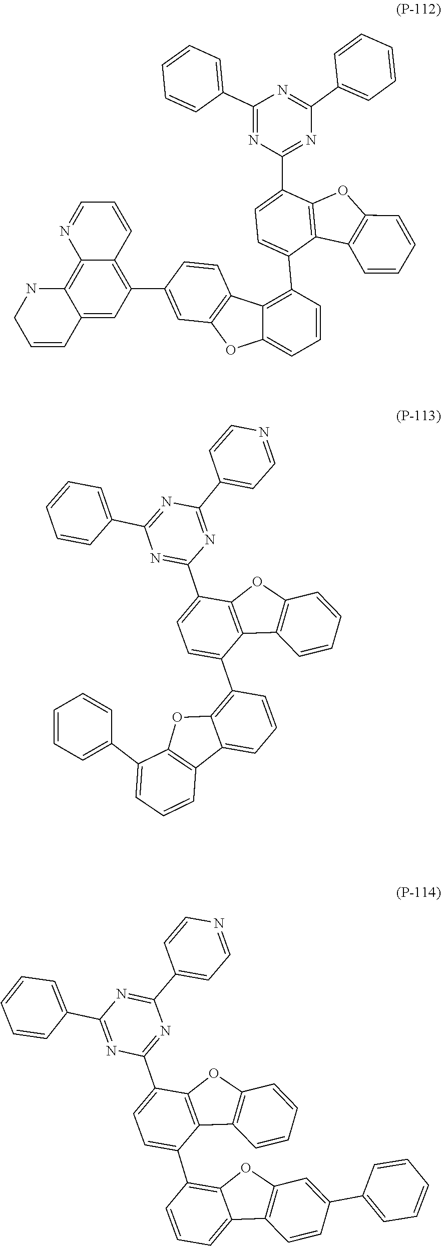 [SMILES: C1=Cc2cc(-c3ccc4c(c3)oc3cccc(-c5ccc(-c6nc(-c7ccccc7)nc(-c7ccccc7)n6)c6oc7ccccc7c56)c34)c3cccnc3c2NC1 ; c1ccc(-c2nc(-c3ccncc3)nc(-c3ccc(-c4cccc5c4oc4c(-c6ccccc6)cccc45)c4c3oc3ccccc34)n2)cc1 ; c1ccc(-c2ccc3c(c2)oc2c(-c4ccc(-c5nc(-c6ccccc6)nc(-c6ccncc6)n5)c5oc6ccccc6c45)cccc23)cc1]

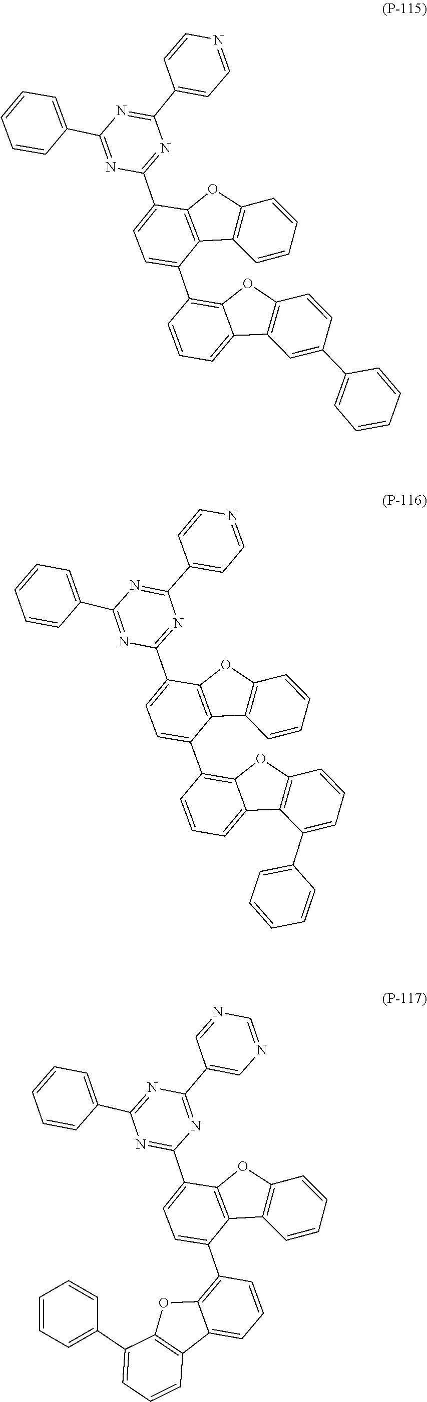 [SMILES: c1ccc(-c2ccc3oc4c(-c5ccc(-c6nc(-c7ccccc7)nc(-c7ccncc7)n6)c6oc7ccccc7c56)cccc4c3c2)cc1 ; c1ccc(-c2nc(-c3ccncc3)nc(-c3ccc(-c4cccc5c4oc4cccc(-c6ccccc6)c45)c4c3oc3ccccc34)n2)cc1 ; c1ccc(-c2nc(-c3cncnc3)nc(-c3ccc(-c4cccc5c4oc4c(-c6ccccc6)cccc45)c4c3oc3ccccc34)n2)cc1]

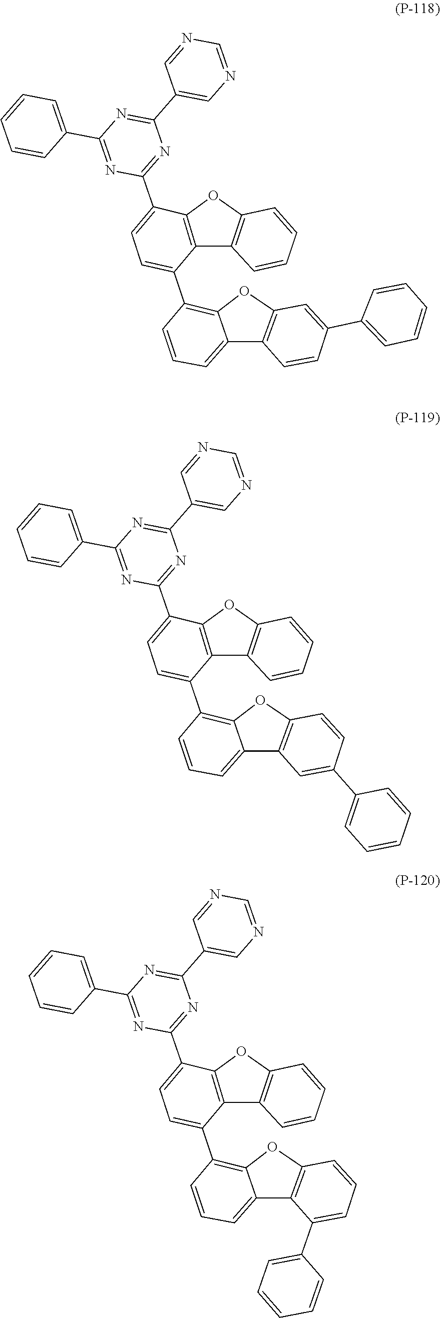 [SMILES: c1ccc(-c2ccc3c(c2)oc2c(-c4ccc(-c5nc(-c6ccccc6)nc(-c6cncnc6)n5)c5oc6ccccc6c45)cccc23)cc1 ; c1ccc(-c2ccc3oc4c(-c5ccc(-c6nc(-c7ccccc7)nc(-c7cncnc7)n6)c6oc7ccccc7c56)cccc4c3c2)cc1 ; c1ccc(-c2nc(-c3cncnc3)nc(-c3ccc(-c4cccc5c4oc4cccc(-c6ccccc6)c45)c4c3oc3ccccc34)n2)cc1]

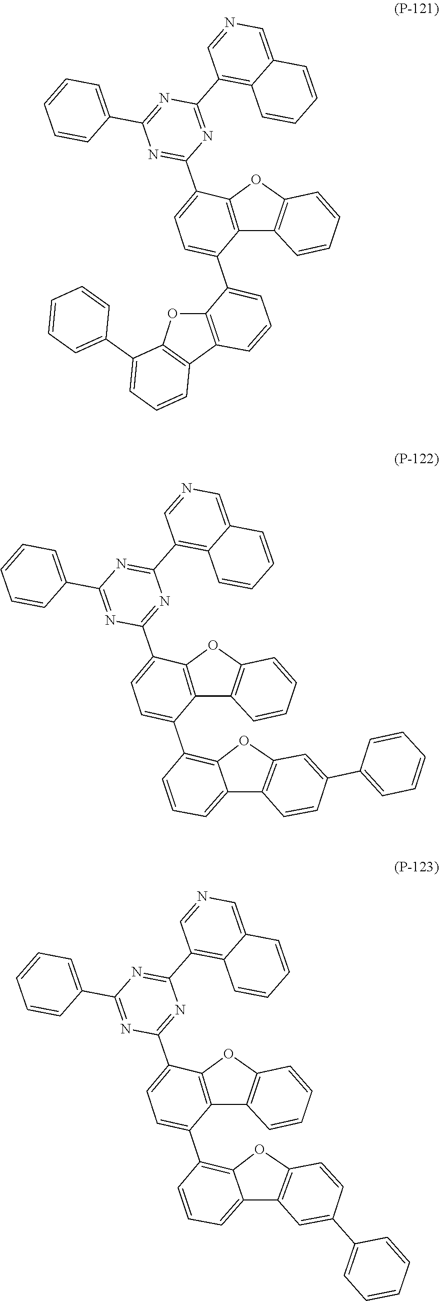 [SMILES: c1ccc(-c2nc(-c3cncc4ccccc34)nc(-c3ccc(-c4cccc5c4oc4c(-c6ccccc6)cccc45)c4c3oc3ccccc34)n2)cc1 ; c1ccc(-c2ccc3c(c2)oc2c(-c4ccc(-c5nc(-c6ccccc6)nc(-c6cncc7ccccc67)n5)c5oc6ccccc6c45)cccc23)cc1 ; c1ccc(-c2ccc3oc4c(-c5ccc(-c6nc(-c7ccccc7)nc(-c7cncc8ccccc78)n6)c6oc7ccccc7c56)cccc4c3c2)cc1]

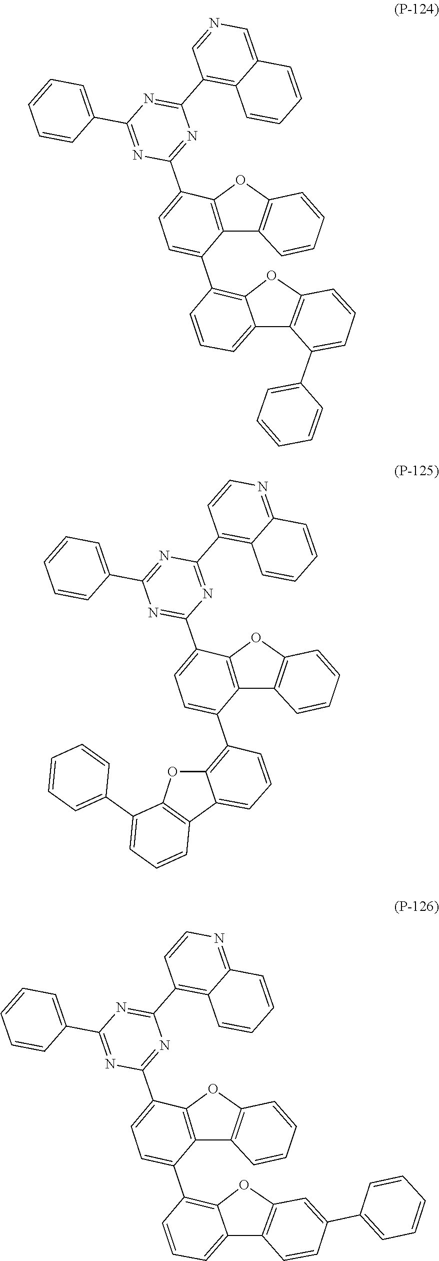

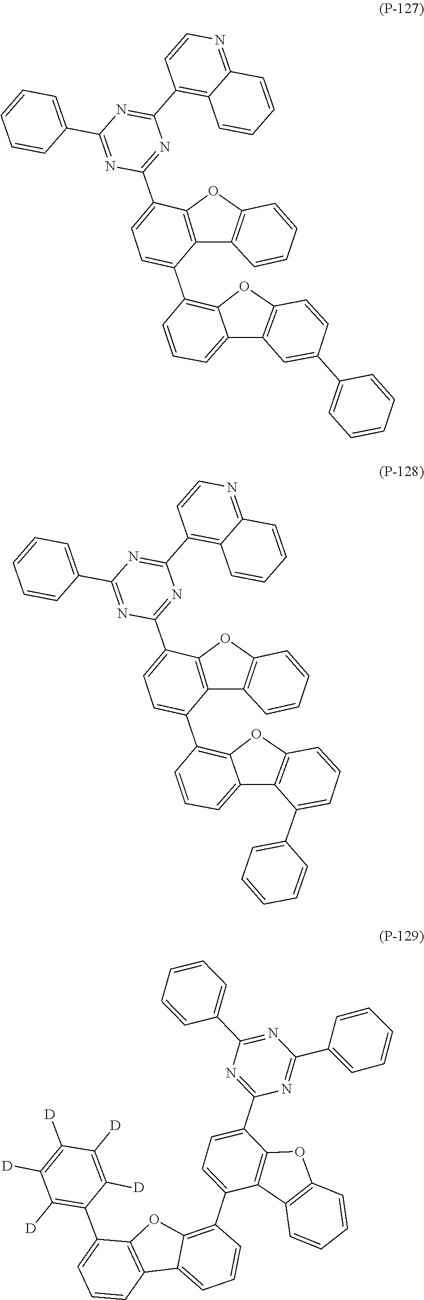 [SMILES: c1ccc(-c2ccc3oc4c(-c5ccc(-c6nc(-c7ccccc7)nc(-c7ccnc8ccccc78)n6)c6oc7ccccc7c56)cccc4c3c2)cc1 ; c1ccc(-c2nc(-c3ccnc4ccccc34)nc(-c3ccc(-c4cccc5c4oc4cccc(-c6ccccc6)c45)c4c3oc3ccccc34)n2)cc1 ; [2H]c1c([2H])c([2H])c(-c2cccc3c2oc2c(-c4ccc(-c5nc(-c6ccccc6)nc(-c6ccccc6)n5)c5oc6ccccc6c45)cccc23)c([2H])c1[2H]]

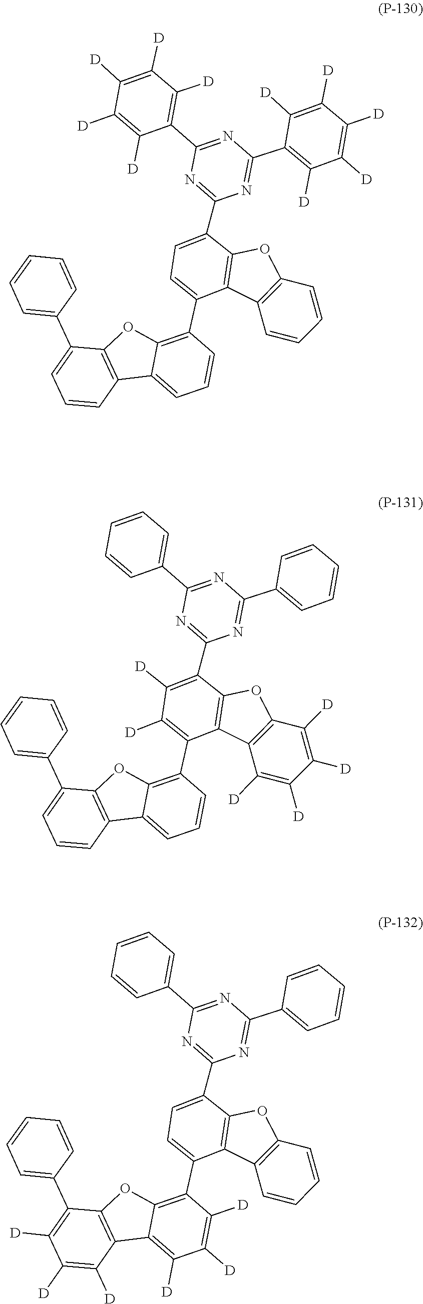 [SMILES: [2H]c1c([2H])c([2H])c(-c2nc(-c3c([2H])c([2H])c([2H])c([2H])c3[2H])nc(-c3ccc(-c4cccc5c4oc4c(-c6ccccc6)cccc45)c4c3oc3ccccc34)n2)c([2H])c1[2H] ; [2H]c1c([2H])c([2H])c2c(oc3c(-c4nc(-c5ccccc5)nc(-c5ccccc5)n4)c([2H])c([2H])c(-c4cccc5c4oc4c(-c6ccccc6)cccc45)c32)c1[2H] ; [2H]c1c([2H])c([2H])c2c(oc3c(-c4ccc(-c5nc(-c6ccccc6)nc(-c6ccccc6)n5)c5oc6ccccc6c45)c([2H])c([2H])c([2H])c32)c1-c1ccccc1]

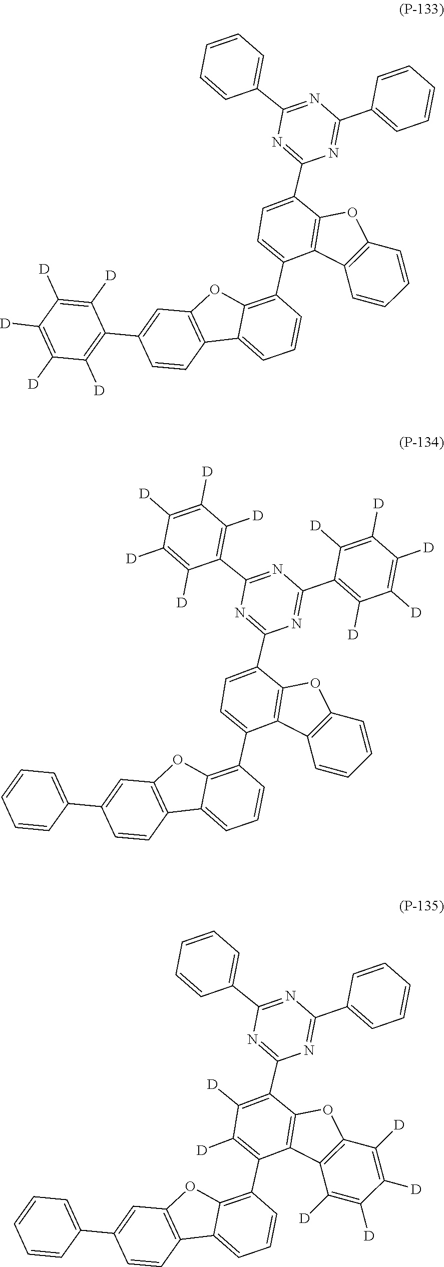 [SMILES: [2H]c1c([2H])c([2H])c(-c2ccc3c(c2)oc2c(-c4ccc(-c5nc(-c6ccccc6)nc(-c6ccccc6)n5)c5oc6ccccc6c45)cccc23)c([2H])c1[2H] ; [2H]c1c([2H])c([2H])c(-c2nc(-c3c([2H])c([2H])c([2H])c([2H])c3[2H])nc(-c3ccc(-c4cccc5c4oc4cc(-c6ccccc6)ccc45)c4c3oc3ccccc34)n2)c([2H])c1[2H] ; [2H]c1c([2H])c([2H])c2c(oc3c(-c4nc(-c5ccccc5)nc(-c5ccccc5)n4)c([2H])c([2H])c(-c4cccc5c4oc4cc(-c6ccccc6)ccc45)c32)c1[2H]]

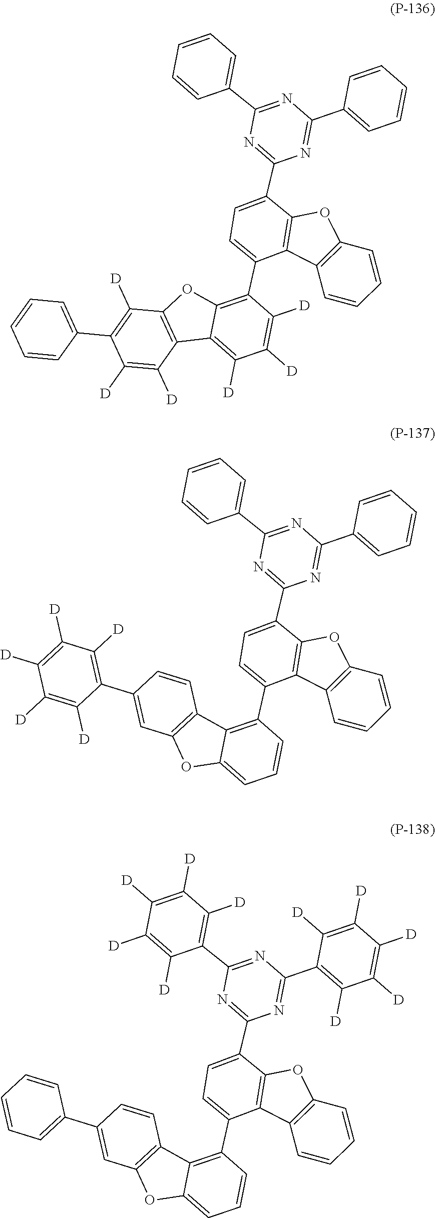 [SMILES: [2H]c1c([2H])c([2H])c2c(oc3c([2H])c(-c4ccccc4)c([2H])c([2H])c32)c1-c1ccc(-c2nc(-c3ccccc3)nc(-c3ccccc3)n2)c2oc3ccccc3c12 ; [2H]c1c([2H])c([2H])c(-c2ccc3c(c2)oc2cccc(-c4ccc(-c5nc(-c6ccccc6)nc(-c6ccccc6)n5)c5oc6ccccc6c45)c23)c([2H])c1[2H] ; [2H]c1c([2H])c([2H])c(-c2nc(-c3c([2H])c([2H])c([2H])c([2H])c3[2H])nc(-c3ccc(-c4cccc5oc6cc(-c7ccccc7)ccc6c45)c4c3oc3ccccc34)n2)c([2H])c1[2H]]

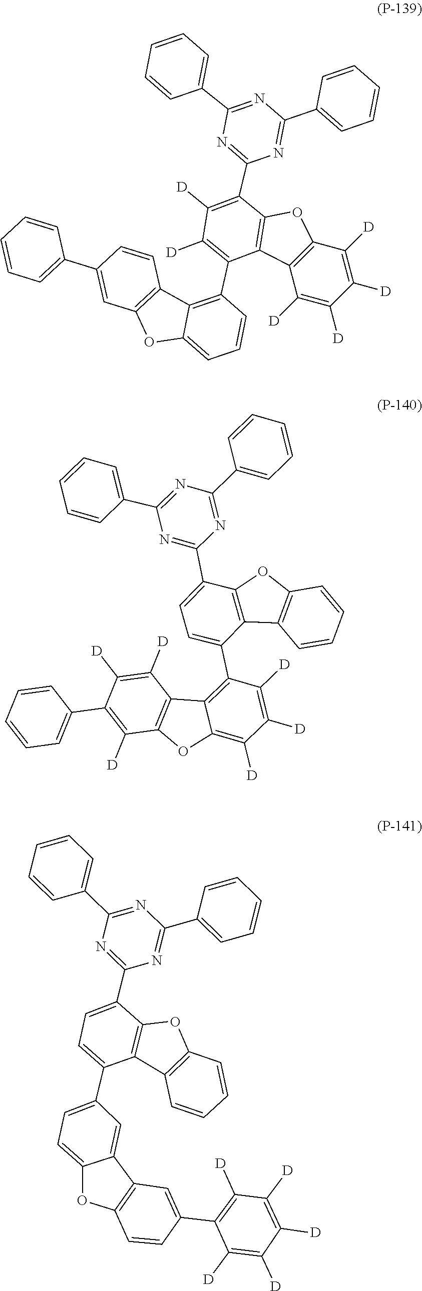 [SMILES: [2H]c1c([2H])c([2H])c2c(oc3c(-c4nc(-c5ccccc5)nc(-c5ccccc5)n4)c([2H])c([2H])c(-c4cccc5oc6cc(-c7ccccc7)ccc6c45)c32)c1[2H] ; [2H]c1c([2H])c(-c2ccc(-c3nc(-c4ccccc4)nc(-c4ccccc4)n3)c3oc4ccccc4c23)c2c(oc3c([2H])c(-c4ccccc4)c([2H])c([2H])c32)c1[2H] ; [2H]c1c([2H])c([2H])c(-c2ccc3oc4ccc(-c5ccc(-c6nc(-c7ccccc7)nc(-c7ccccc7)n6)c6oc7ccccc7c56)cc4c3c2)c([2H])c1[2H]]

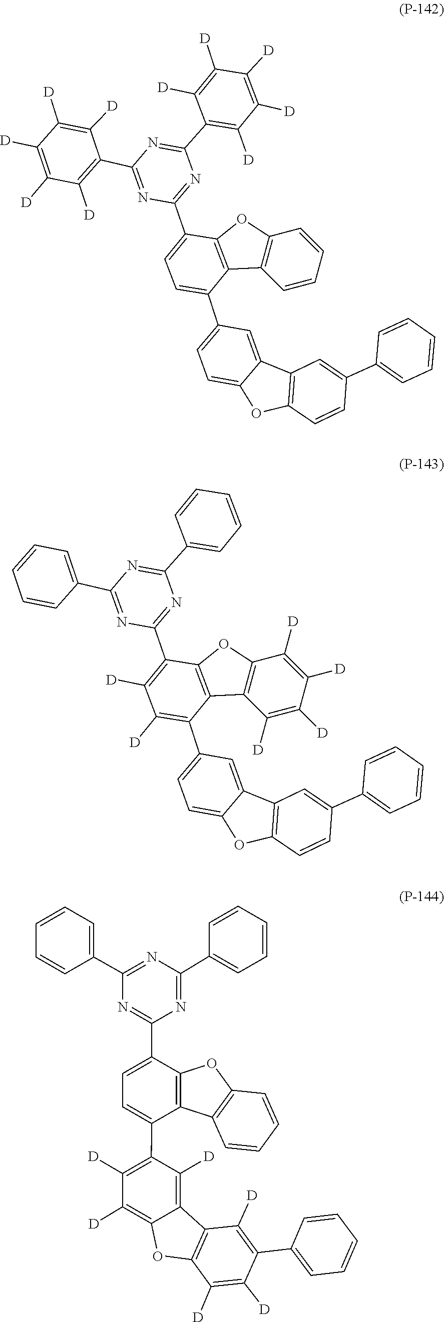 [SMILES: [2H]c1c([2H])c([2H])c(-c2nc(-c3c([2H])c([2H])c([2H])c([2H])c3[2H])nc(-c3ccc(-c4ccc5oc6ccc(-c7ccccc7)cc6c5c4)c4c3oc3ccccc34)n2)c([2H])c1[2H] ; [2H]c1c([2H])c([2H])c2c(oc3c(-c4nc(-c5ccccc5)nc(-c5ccccc5)n4)c([2H])c([2H])c(-c4ccc5oc6ccc(-c7ccccc7)cc6c5c4)c32)c1[2H] ; [2H]c1c(-c2ccccc2)c([2H])c2c(oc3c([2H])c([2H])c(-c4ccc(-c5nc(-c6ccccc6)nc(-c6ccccc6)n5)c5oc6ccccc6c45)c([2H])c32)c1[2H]]

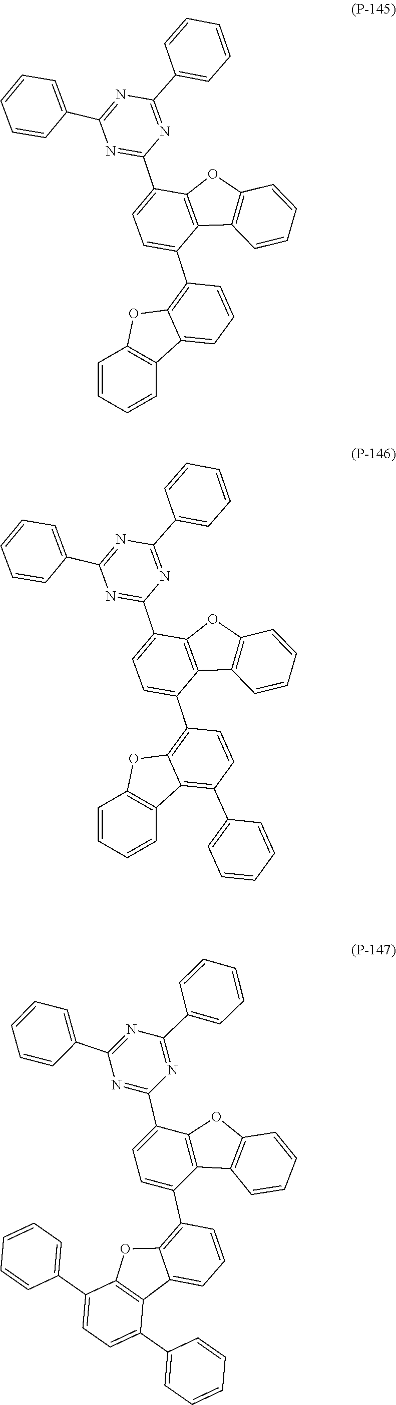 [SMILES: c1ccc(-c2nc(-c3ccccc3)nc(-c3ccc(-c4cccc5c4oc4ccccc45)c4c3oc3ccccc34)n2)cc1 ; c1ccc(-c2nc(-c3ccccc3)nc(-c3ccc(-c4ccc(-c5ccccc5)c5c4oc4ccccc45)c4c3oc3ccccc34)n2)cc1 ; c1ccc(-c2nc(-c3ccccc3)nc(-c3ccc(-c4cccc5c4oc4c(-c6ccccc6)ccc(-c6ccccc6)c45)c4c3oc3ccccc34)n2)cc1]

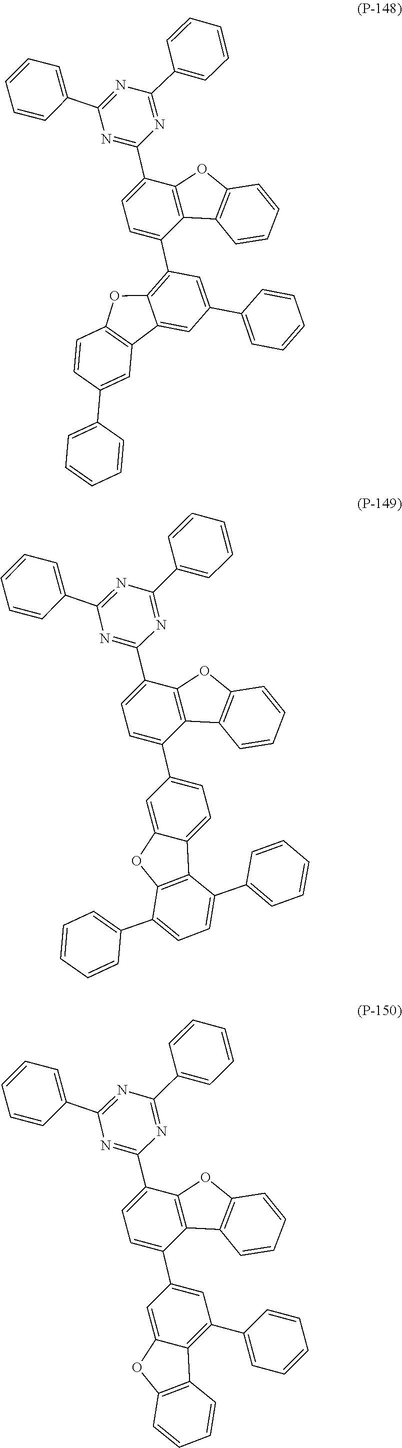 [SMILES: c1ccc(-c2ccc3oc4c(-c5ccc(-c6nc(-c7ccccc7)nc(-c7ccccc7)n6)c6oc7ccccc7c56)cc(-c5ccccc5)cc4c3c2)cc1 ; c1ccc(-c2nc(-c3ccccc3)nc(-c3ccc(-c4ccc5c(c4)oc4c(-c6ccccc6)ccc(-c6ccccc6)c45)c4c3oc3ccccc34)n2)cc1 ; c1ccc(-c2nc(-c3ccccc3)nc(-c3ccc(-c4cc(-c5ccccc5)c5c(c4)oc4ccccc45)c4c3oc3ccccc34)n2)cc1]

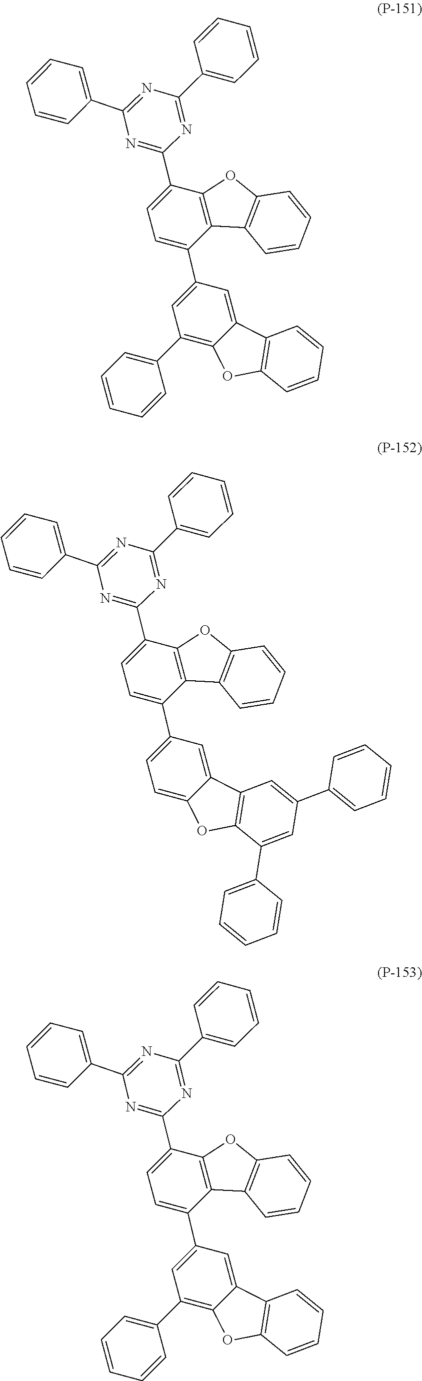

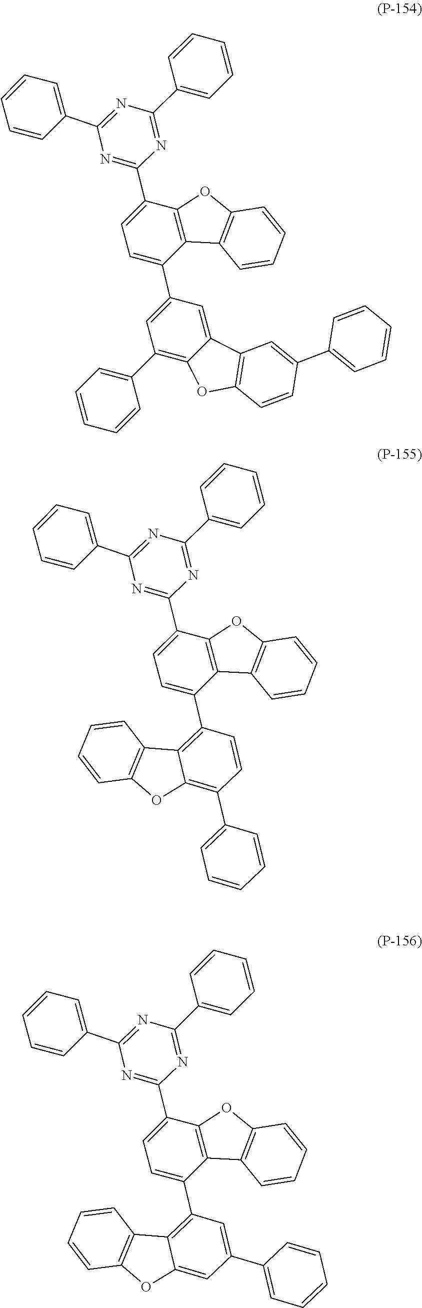 [SMILES: c1ccc(-c2ccc3oc4c(-c5ccccc5)cc(-c5ccc(-c6nc(-c7ccccc7)nc(-c7ccccc7)n6)c6oc7ccccc7c56)cc4c3c2)cc1 ; c1ccc(-c2nc(-c3ccccc3)nc(-c3ccc(-c4ccc(-c5ccccc5)c5oc6ccccc6c45)c4c3oc3ccccc34)n2)cc1 ; c1ccc(-c2cc(-c3ccc(-c4nc(-c5ccccc5)nc(-c5ccccc5)n4)c4oc5ccccc5c34)c3c(c2)oc2ccccc23)cc1]

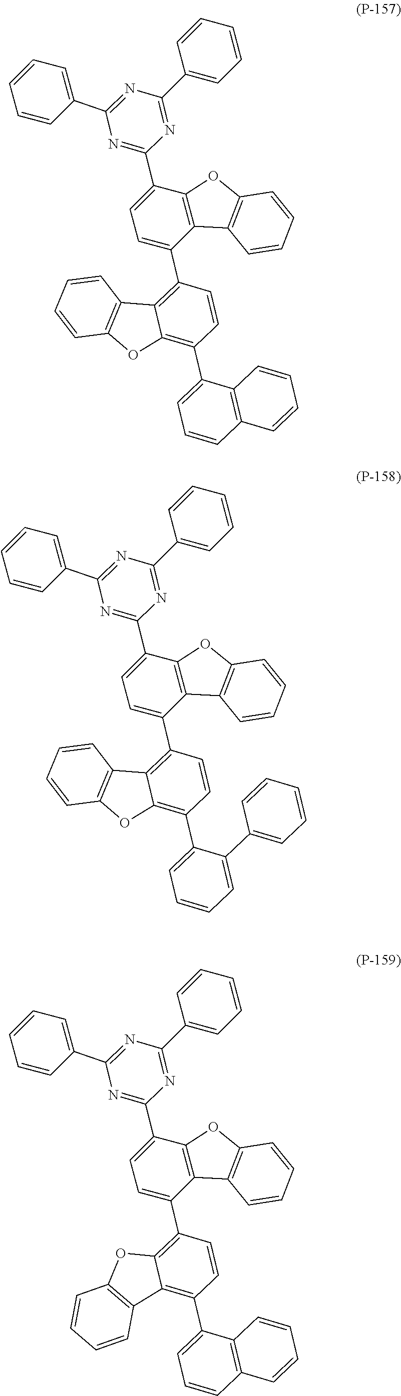 [SMILES: c1ccc(-c2nc(-c3ccccc3)nc(-c3ccc(-c4ccc(-c5cccc6ccccc56)c5oc6ccccc6c45)c4c3oc3ccccc34)n2)cc1 ; c1ccc(-c2nc(-c3ccccc3)nc(-c3ccc(-c4ccc(-c5ccccc5-c5ccccc5)c5oc6ccccc6c45)c4c3oc3ccccc34)n2)cc1 ; c1ccc(-c2nc(-c3ccccc3)nc(-c3ccc(-c4ccc(-c5cccc6ccccc56)c5c4oc4ccccc45)c4c3oc3ccccc34)n2)cc1]

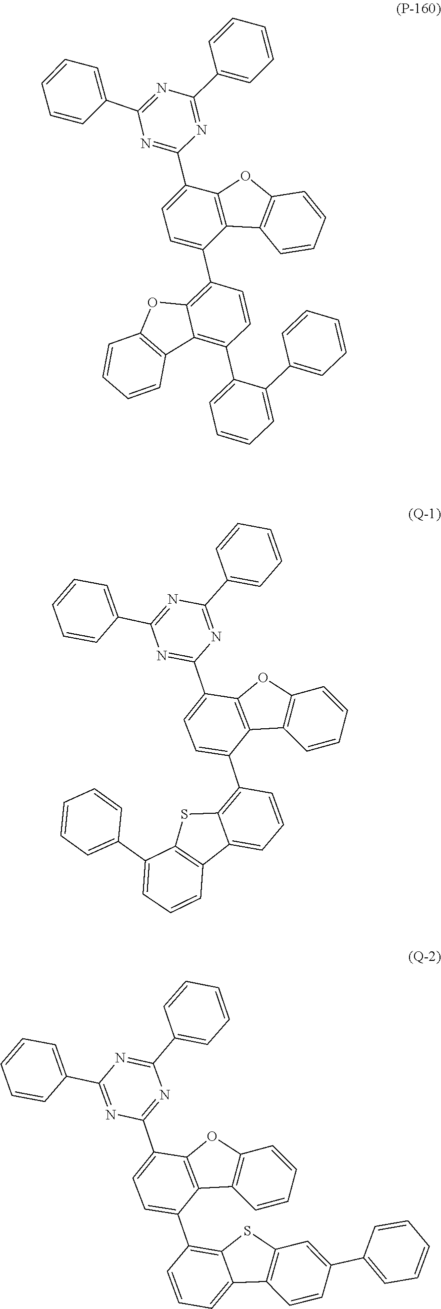 [SMILES: c1ccc(-c2nc(-c3ccccc3)nc(-c3ccc(-c4ccc(-c5ccccc5-c5ccccc5)c5c4oc4ccccc45)c4c3oc3ccccc34)n2)cc1 ; c1ccc(-c2nc(-c3ccccc3)nc(-c3ccc(-c4cccc5c4sc4c(-c6ccccc6)cccc45)c4c3oc3ccccc34)n2)cc1 ; c1ccc(-c2ccc3c(c2)sc2c(-c4ccc(-c5nc(-c6ccccc6)nc(-c6ccccc6)n5)c5oc6ccccc6c45)cccc23)cc1]

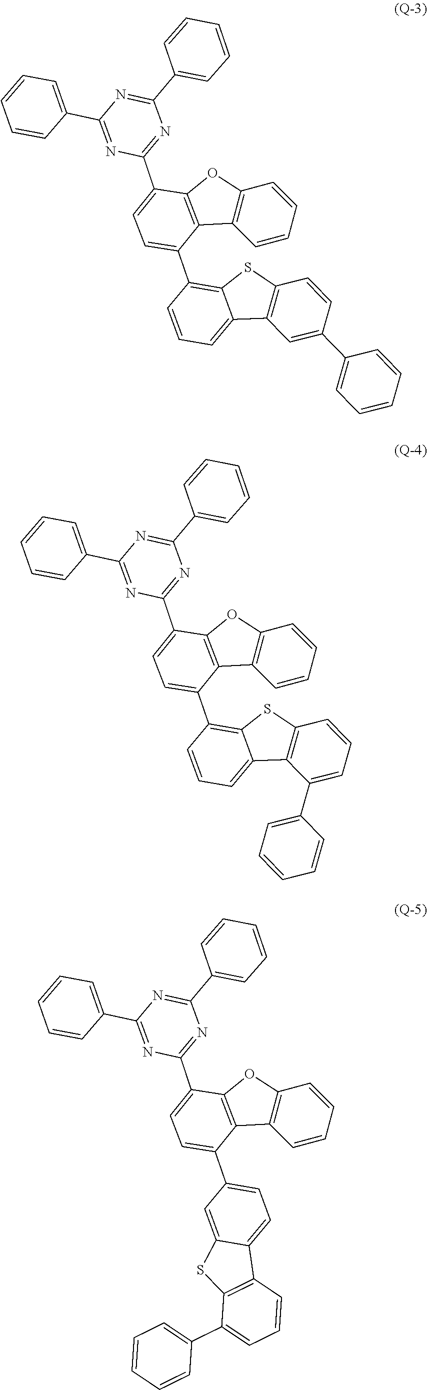 [SMILES: c1ccc(-c2ccc3sc4c(-c5ccc(-c6nc(-c7ccccc7)nc(-c7ccccc7)n6)c6oc7ccccc7c56)cccc4c3c2)cc1 ; c1ccc(-c2nc(-c3ccccc3)nc(-c3ccc(-c4cccc5c4sc4cccc(-c6ccccc6)c45)c4c3oc3ccccc34)n2)cc1 ; c1ccc(-c2nc(-c3ccccc3)nc(-c3ccc(-c4ccc5c(c4)sc4c(-c6ccccc6)cccc45)c4c3oc3ccccc34)n2)cc1]

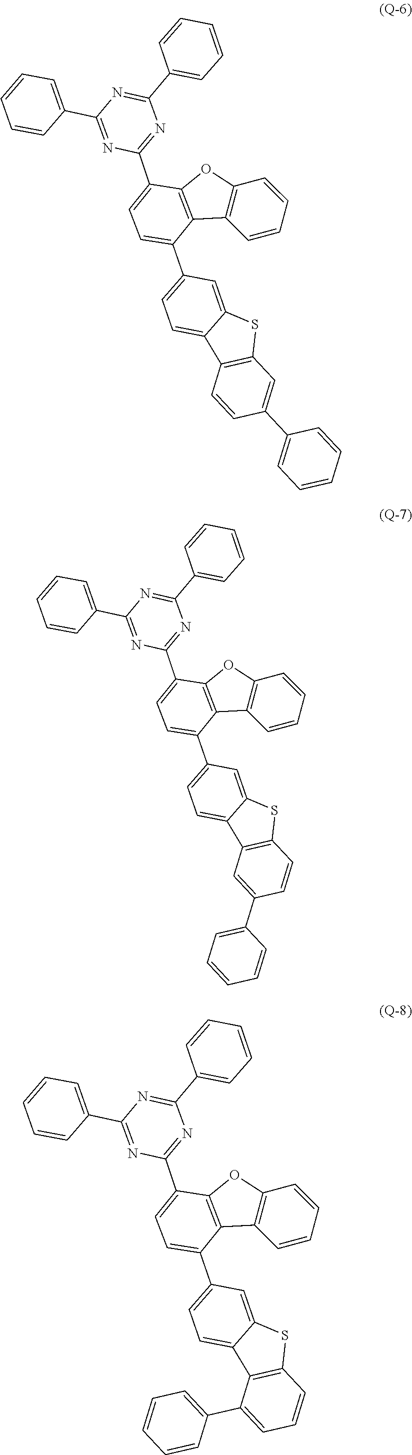

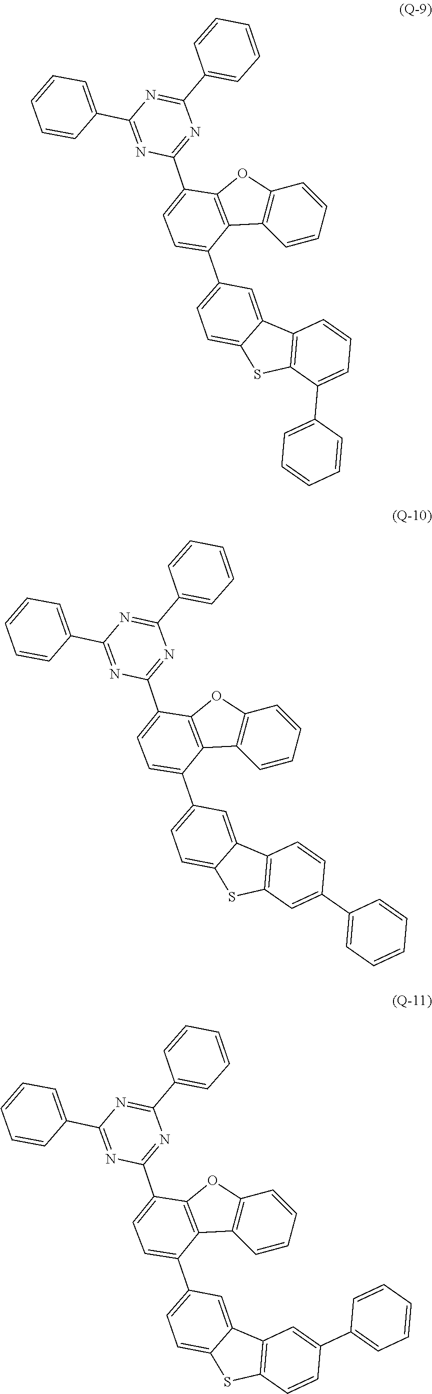 [SMILES: c1ccc(-c2nc(-c3ccccc3)nc(-c3ccc(-c4ccc5sc6c(-c7ccccc7)cccc6c5c4)c4c3oc3ccccc34)n2)cc1 ; c1ccc(-c2ccc3c(c2)sc2ccc(-c4ccc(-c5nc(-c6ccccc6)nc(-c6ccccc6)n5)c5oc6ccccc6c45)cc23)cc1 ; c1ccc(-c2ccc3sc4ccc(-c5ccc(-c6nc(-c7ccccc7)nc(-c7ccccc7)n6)c6oc7ccccc7c56)cc4c3c2)cc1]

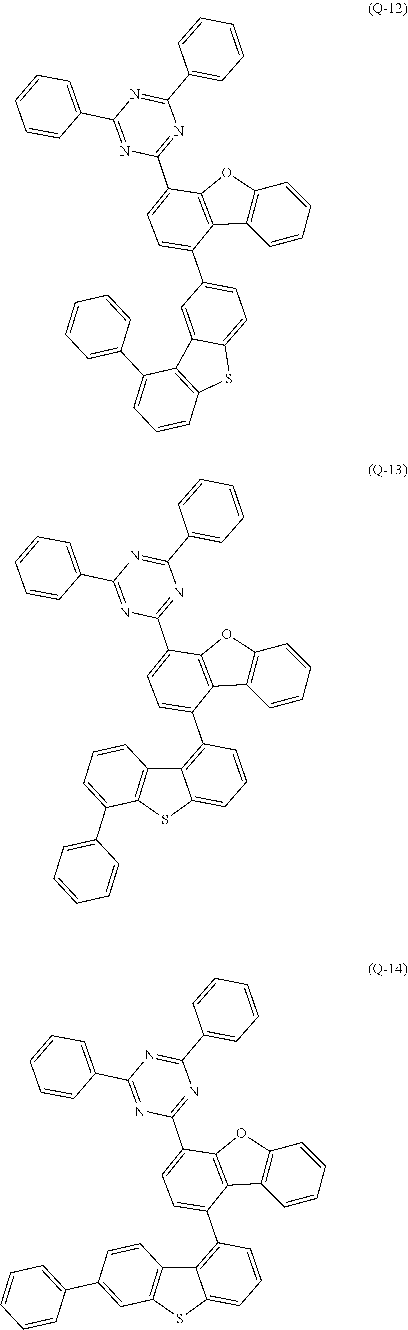 [SMILES: c1ccc(-c2nc(-c3ccccc3)nc(-c3ccc(-c4ccc5sc6cccc(-c7ccccc7)c6c5c4)c4c3oc3ccccc34)n2)cc1 ; c1ccc(-c2nc(-c3ccccc3)nc(-c3ccc(-c4cccc5sc6c(-c7ccccc7)cccc6c45)c4c3oc3ccccc34)n2)cc1 ; c1ccc(-c2ccc3c(c2)sc2cccc(-c4ccc(-c5nc(-c6ccccc6)nc(-c6ccccc6)n5)c5oc6ccccc6c45)c23)cc1]

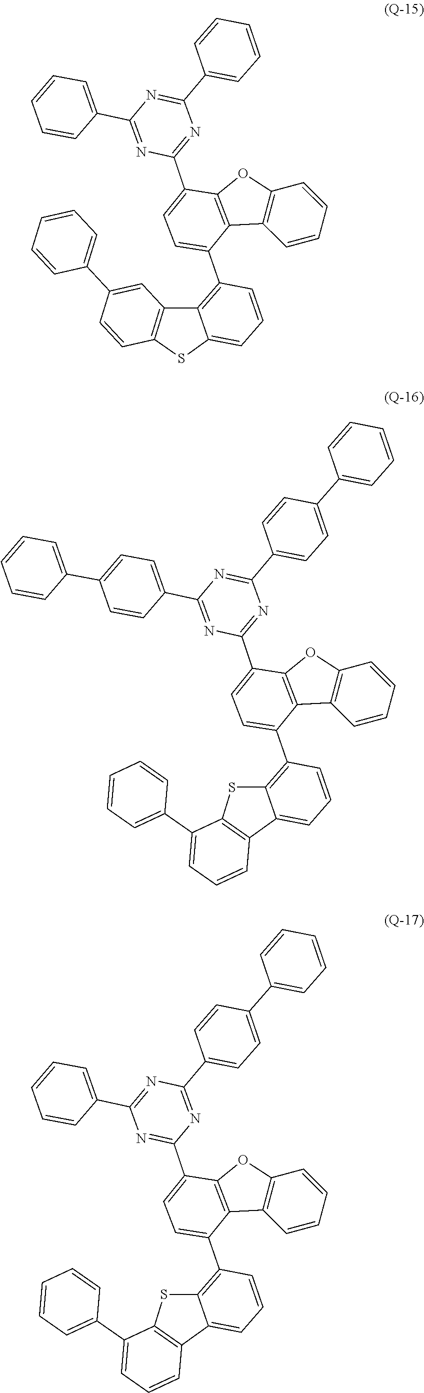 [SMILES: c1ccc(-c2ccc3sc4cccc(-c5ccc(-c6nc(-c7ccccc7)nc(-c7ccccc7)n6)c6oc7ccccc7c56)c4c3c2)cc1 ; c1ccc(-c2ccc(-c3nc(-c4ccc(-c5ccccc5)cc4)nc(-c4ccc(-c5cccc6c5sc5c(-c7ccccc7)cccc56)c5c4oc4ccccc45)n3)cc2)cc1 ; c1ccc(-c2ccc(-c3nc(-c4ccccc4)nc(-c4ccc(-c5cccc6c5sc5c(-c7ccccc7)cccc56)c5c4oc4ccccc45)n3)cc2)cc1]

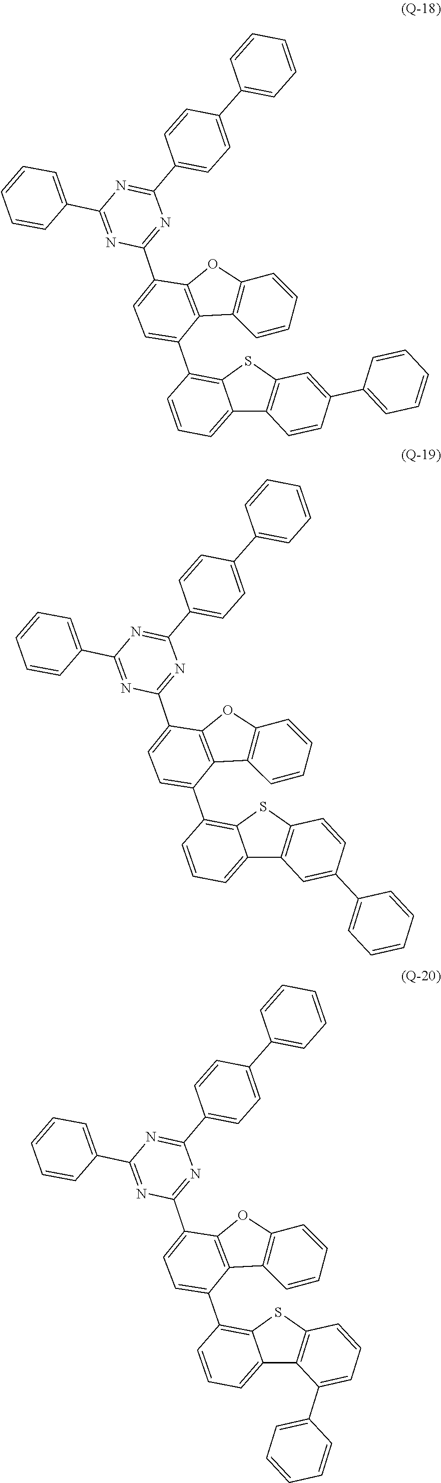 [SMILES: c1ccc(-c2ccc(-c3nc(-c4ccccc4)nc(-c4ccc(-c5cccc6c5sc5cc(-c7ccccc7)ccc56)c5c4oc4ccccc45)n3)cc2)cc1 ; c1ccc(-c2ccc(-c3nc(-c4ccccc4)nc(-c4ccc(-c5cccc6c5sc5ccc(-c7ccccc7)cc56)c5c4oc4ccccc45)n3)cc2)cc1 ; c1ccc(-c2ccc(-c3nc(-c4ccccc4)nc(-c4ccc(-c5cccc6c5sc5cccc(-c7ccccc7)c56)c5c4oc4ccccc45)n3)cc2)cc1]

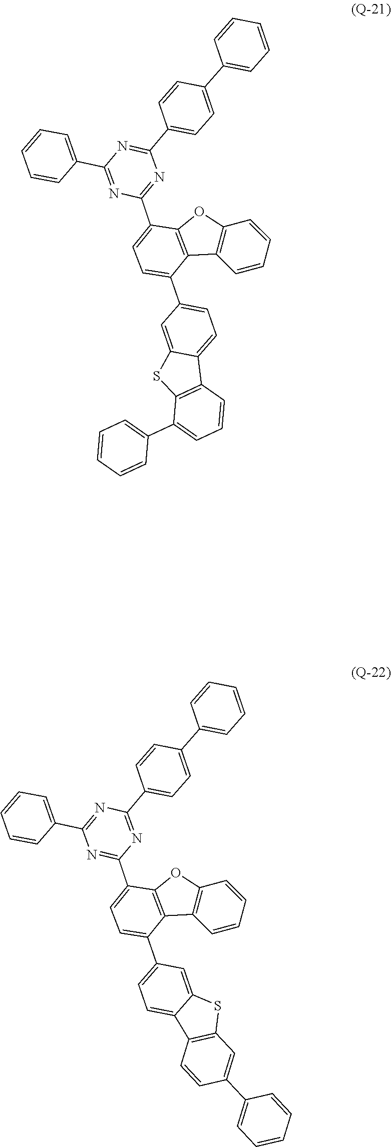 [SMILES: c1ccc(-c2ccc(-c3nc(-c4ccccc4)nc(-c4ccc(-c5ccc6c(c5)sc5c(-c7ccccc7)cccc56)c5c4oc4ccccc45)n3)cc2)cc1 ; c1ccc(-c2ccc(-c3nc(-c4ccccc4)nc(-c4ccc(-c5ccc6c(c5)sc5cc(-c7ccccc7)ccc56)c5c4oc4ccccc45)n3)cc2)cc1]

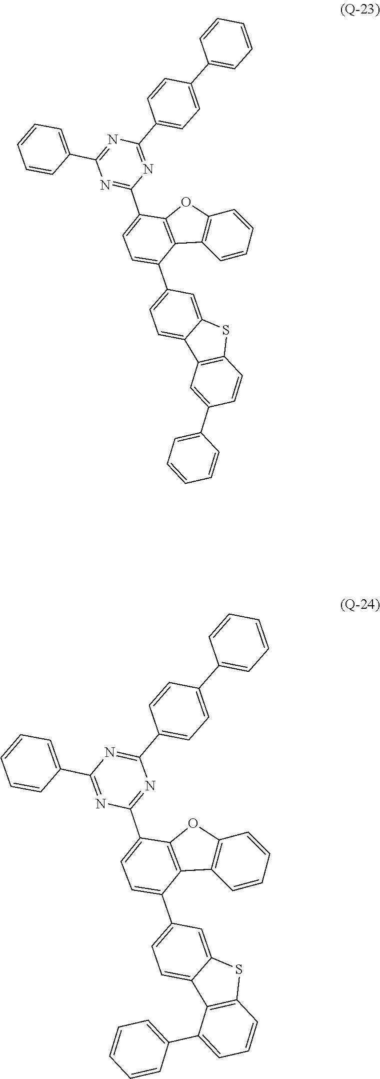 [SMILES: c1ccc(-c2ccc(-c3nc(-c4ccccc4)nc(-c4ccc(-c5ccc6c(c5)sc5ccc(-c7ccccc7)cc56)c5c4oc4ccccc45)n3)cc2)cc1 ; c1ccc(-c2ccc(-c3nc(-c4ccccc4)nc(-c4ccc(-c5ccc6c(c5)sc5cccc(-c7ccccc7)c56)c5c4oc4ccccc45)n3)cc2)cc1]

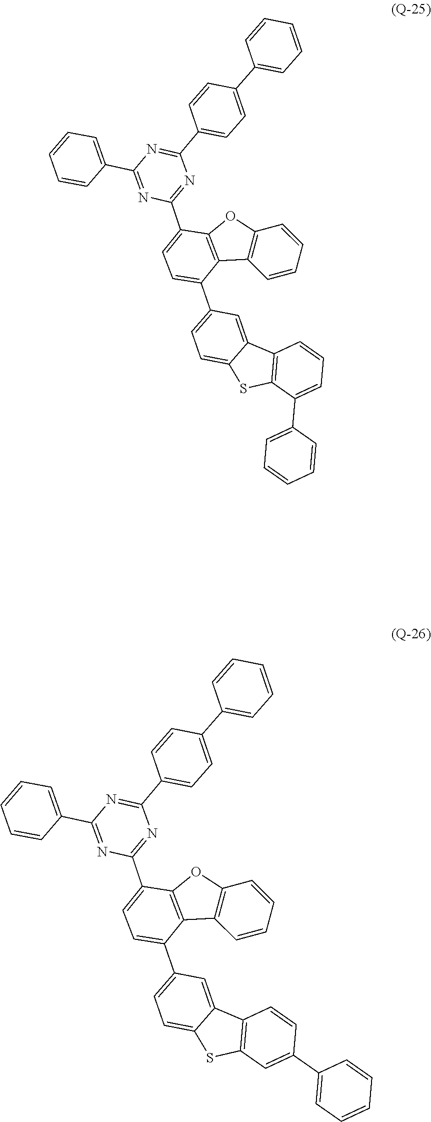 [SMILES: c1ccc(-c2ccc(-c3nc(-c4ccccc4)nc(-c4ccc(-c5ccc6sc7c(-c8ccccc8)cccc7c6c5)c5c4oc4ccccc45)n3)cc2)cc1 ; c1ccc(-c2ccc(-c3nc(-c4ccccc4)nc(-c4ccc(-c5ccc6sc7cc(-c8ccccc8)ccc7c6c5)c5c4oc4ccccc45)n3)cc2)cc1]

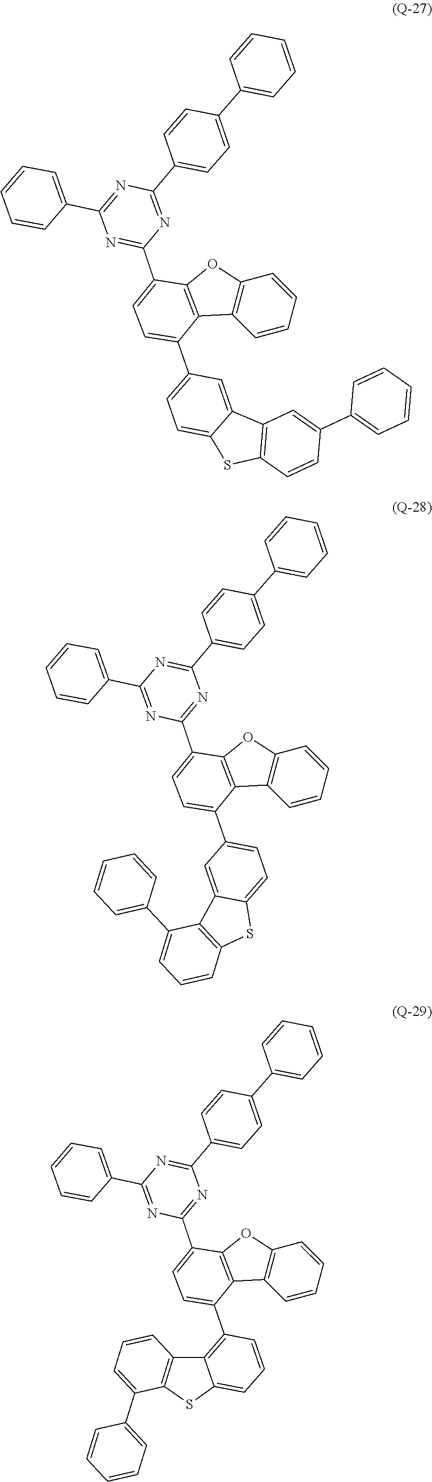

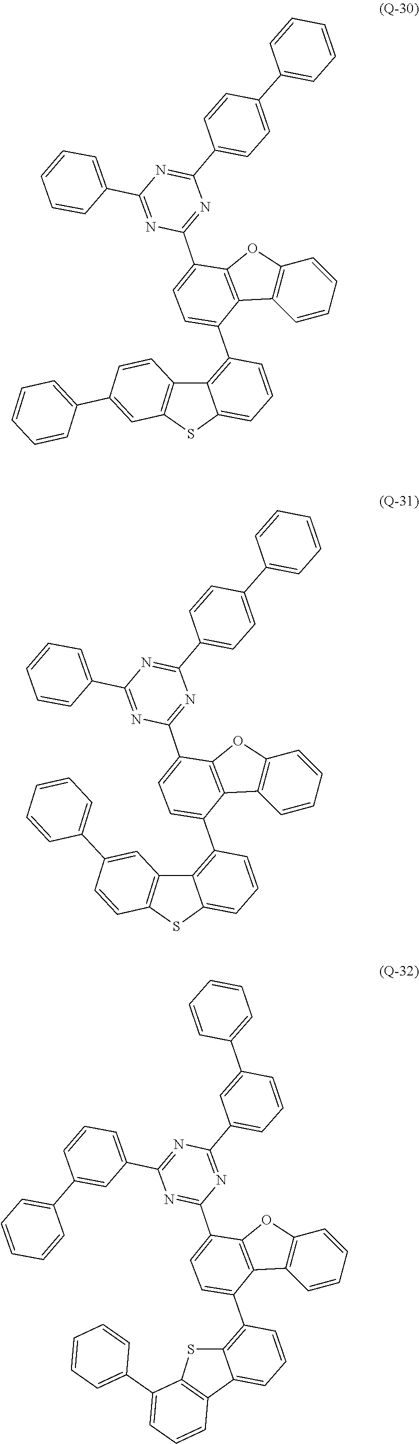 [SMILES: c1ccc(-c2ccc(-c3nc(-c4ccccc4)nc(-c4ccc(-c5cccc6sc7cc(-c8ccccc8)ccc7c56)c5c4oc4ccccc45)n3)cc2)cc1 ; c1ccc(-c2ccc(-c3nc(-c4ccccc4)nc(-c4ccc(-c5cccc6sc7ccc(-c8ccccc8)cc7c56)c5c4oc4ccccc45)n3)cc2)cc1 ; c1ccc(-c2cccc(-c3nc(-c4cccc(-c5ccccc5)c4)nc(-c4ccc(-c5cccc6c5sc5c(-c7ccccc7)cccc56)c5c4oc4ccccc45)n3)c2)cc1]

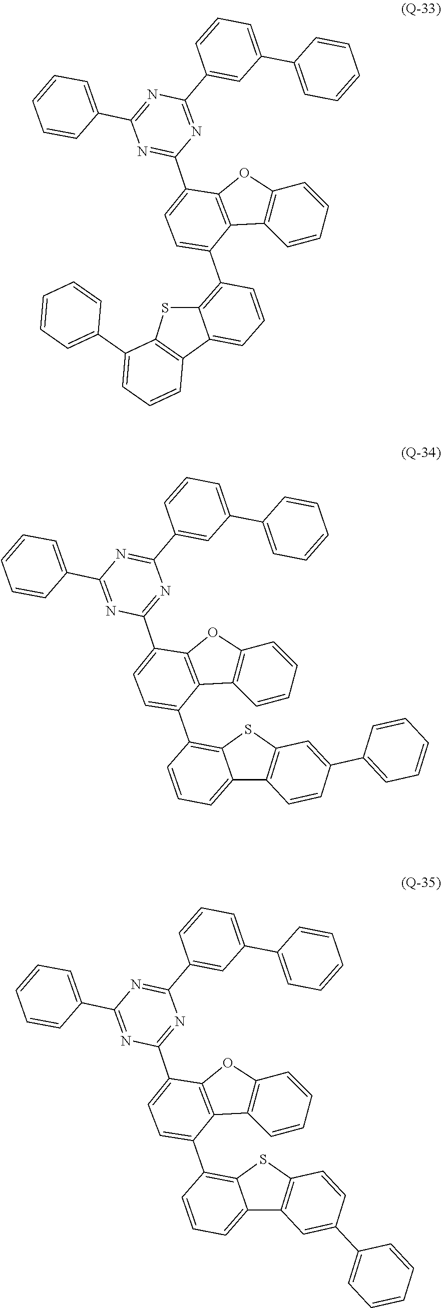 [SMILES: c1ccc(-c2cccc(-c3nc(-c4ccccc4)nc(-c4ccc(-c5cccc6c5sc5c(-c7ccccc7)cccc56)c5c4oc4ccccc45)n3)c2)cc1 ; c1ccc(-c2cccc(-c3nc(-c4ccccc4)nc(-c4ccc(-c5cccc6c5sc5cc(-c7ccccc7)ccc56)c5c4oc4ccccc45)n3)c2)cc1 ; c1ccc(-c2cccc(-c3nc(-c4ccccc4)nc(-c4ccc(-c5cccc6c5sc5ccc(-c7ccccc7)cc56)c5c4oc4ccccc45)n3)c2)cc1]

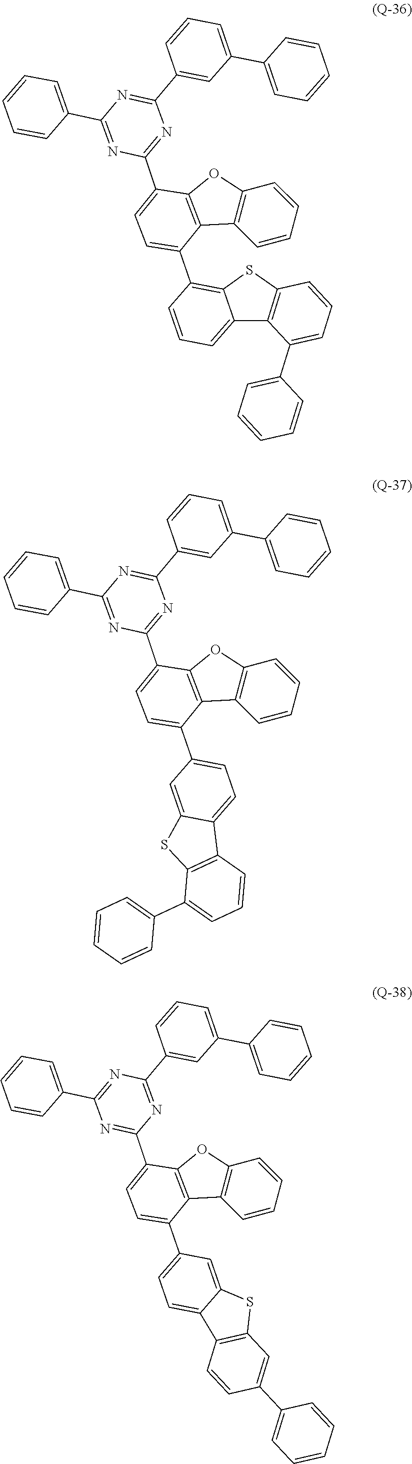 [SMILES: c1ccc(-c2cccc(-c3nc(-c4ccccc4)nc(-c4ccc(-c5cccc6c5sc5cccc(-c7ccccc7)c56)c5c4oc4ccccc45)n3)c2)cc1 ; c1ccc(-c2cccc(-c3nc(-c4ccccc4)nc(-c4ccc(-c5ccc6c(c5)sc5c(-c7ccccc7)cccc56)c5c4oc4ccccc45)n3)c2)cc1 ; c1ccc(-c2cccc(-c3nc(-c4ccccc4)nc(-c4ccc(-c5ccc6c(c5)sc5cc(-c7ccccc7)ccc56)c5c4oc4ccccc45)n3)c2)cc1]

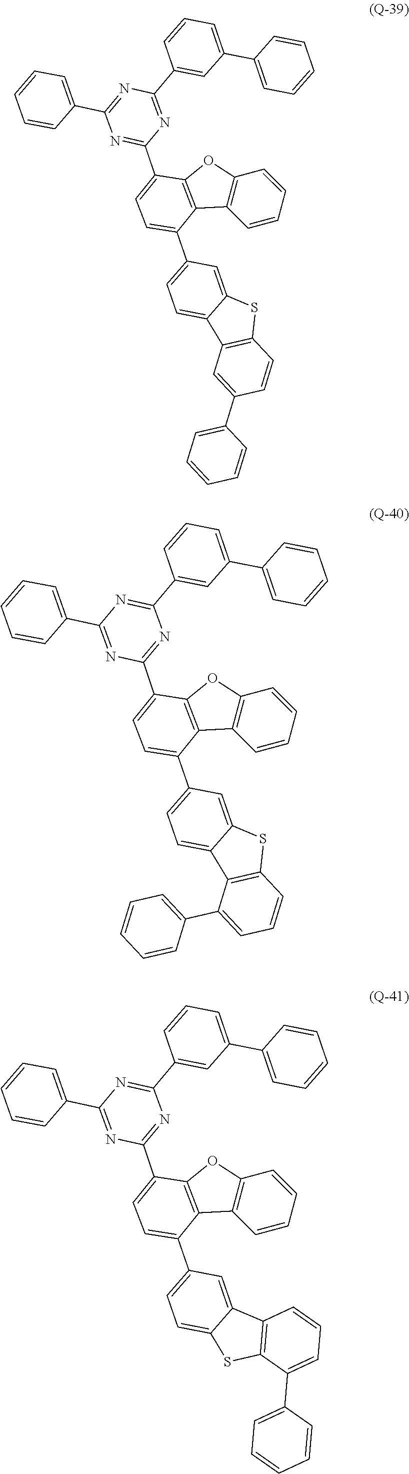

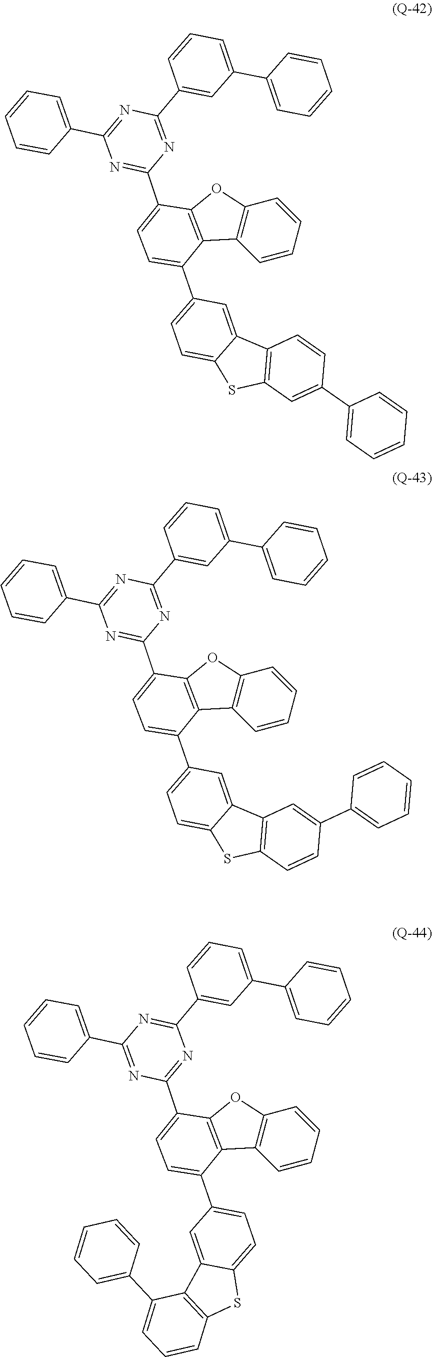 [SMILES: c1ccc(-c2cccc(-c3nc(-c4ccccc4)nc(-c4ccc(-c5ccc6sc7cc(-c8ccccc8)ccc7c6c5)c5c4oc4ccccc45)n3)c2)cc1 ; c1ccc(-c2cccc(-c3nc(-c4ccccc4)nc(-c4ccc(-c5ccc6sc7ccc(-c8ccccc8)cc7c6c5)c5c4oc4ccccc45)n3)c2)cc1 ; c1ccc(-c2cccc(-c3nc(-c4ccccc4)nc(-c4ccc(-c5ccc6sc7cccc(-c8ccccc8)c7c6c5)c5c4oc4ccccc45)n3)c2)cc1]

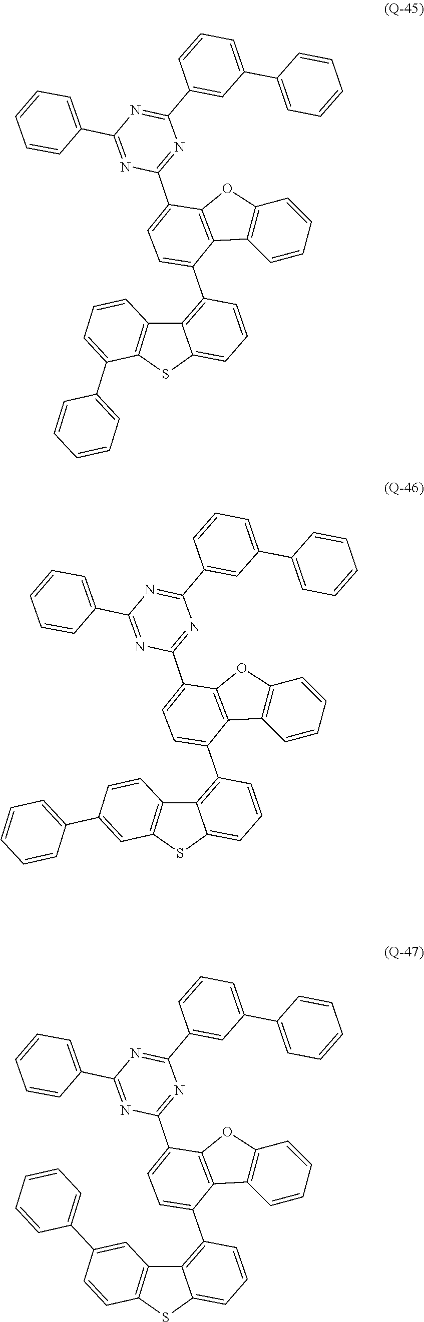 [SMILES: c1ccc(-c2cccc(-c3nc(-c4ccccc4)nc(-c4ccc(-c5cccc6sc7c(-c8ccccc8)cccc7c56)c5c4oc4ccccc45)n3)c2)cc1 ; c1ccc(-c2cccc(-c3nc(-c4ccccc4)nc(-c4ccc(-c5cccc6sc7cc(-c8ccccc8)ccc7c56)c5c4oc4ccccc45)n3)c2)cc1 ; c1ccc(-c2cccc(-c3nc(-c4ccccc4)nc(-c4ccc(-c5cccc6sc7ccc(-c8ccccc8)cc7c56)c5c4oc4ccccc45)n3)c2)cc1]

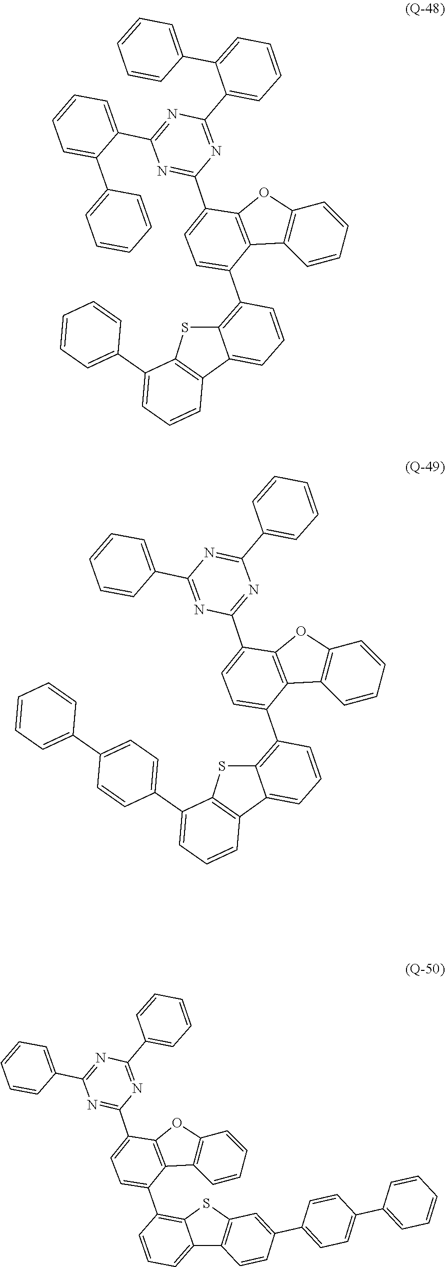 [SMILES: c1ccc(-c2ccccc2-c2nc(-c3ccccc3-c3ccccc3)nc(-c3ccc(-c4cccc5c4sc4c(-c6ccccc6)cccc45)c4c3oc3ccccc34)n2)cc1 ; c1ccc(-c2ccc(-c3cccc4c3sc3c(-c5ccc(-c6nc(-c7ccccc7)nc(-c7ccccc7)n6)c6oc7ccccc7c56)cccc34)cc2)cc1 ; c1ccc(-c2ccc(-c3ccc4c(c3)sc3c(-c5ccc(-c6nc(-c7ccccc7)nc(-c7ccccc7)n6)c6oc7ccccc7c56)cccc34)cc2)cc1]

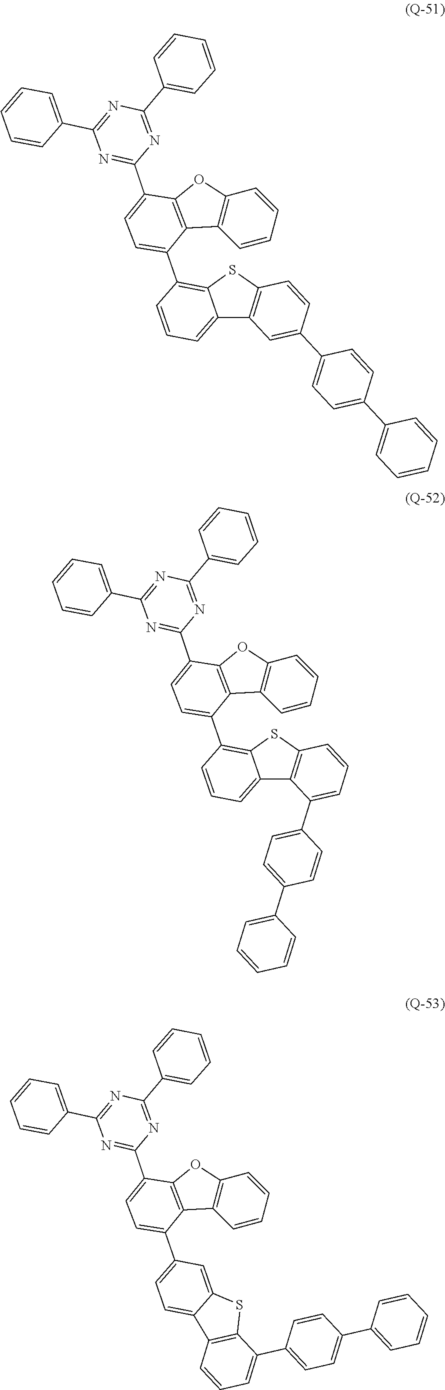 [SMILES: c1ccc(-c2ccc(-c3ccc4sc5c(-c6ccc(-c7nc(-c8ccccc8)nc(-c8ccccc8)n7)c7oc8ccccc8c67)cccc5c4c3)cc2)cc1 ; c1ccc(-c2ccc(-c3cccc4sc5c(-c6ccc(-c7nc(-c8ccccc8)nc(-c8ccccc8)n7)c7oc8ccccc8c67)cccc5c34)cc2)cc1 ; c1ccc(-c2ccc(-c3cccc4c3sc3cc(-c5ccc(-c6nc(-c7ccccc7)nc(-c7ccccc7)n6)c6oc7ccccc7c56)ccc34)cc2)cc1]

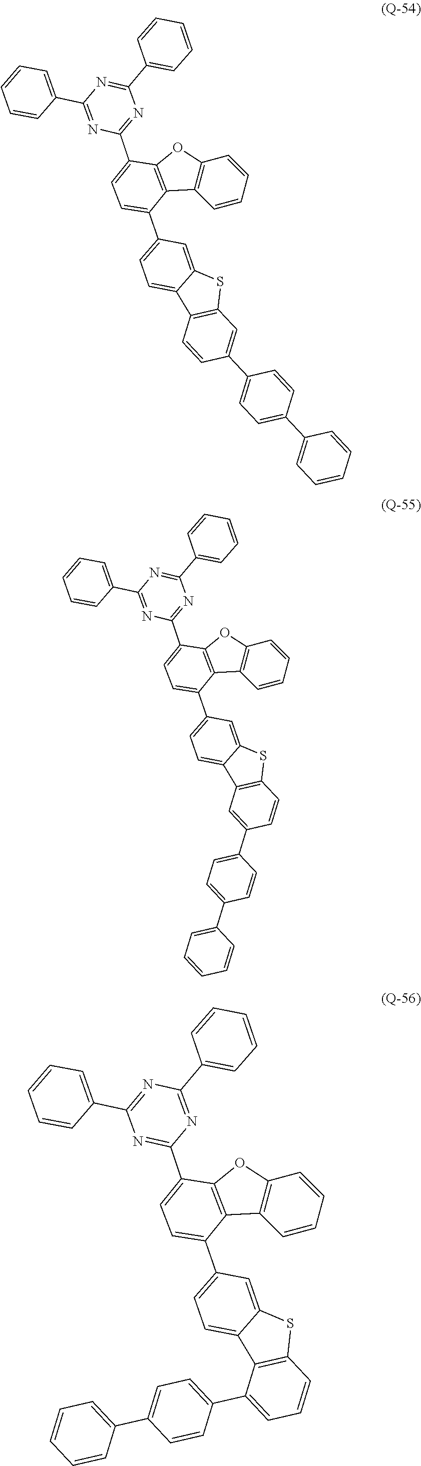

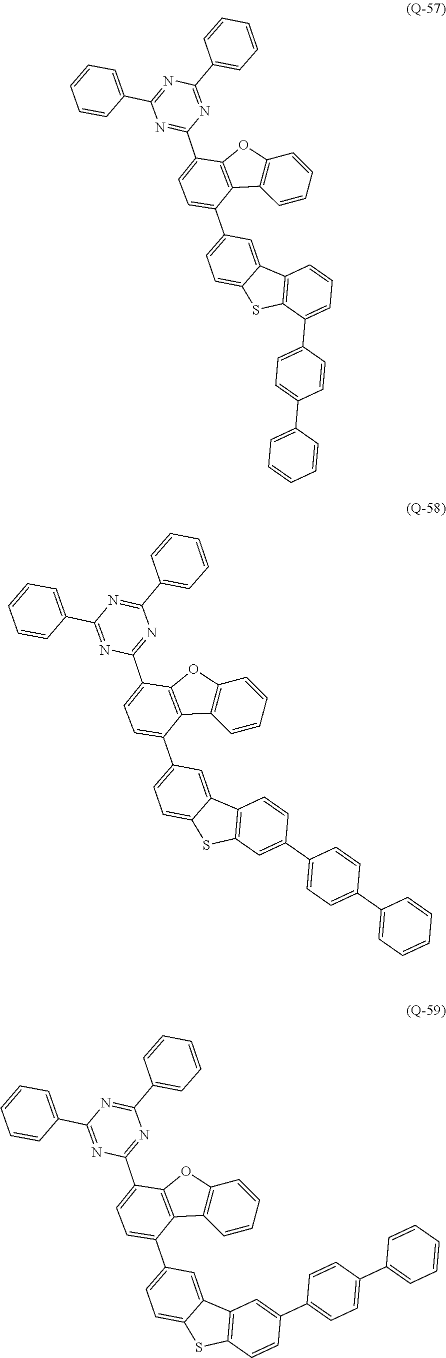 [SMILES: c1ccc(-c2ccc(-c3cccc4c3sc3ccc(-c5ccc(-c6nc(-c7ccccc7)nc(-c7ccccc7)n6)c6oc7ccccc7c56)cc34)cc2)cc1 ; c1ccc(-c2ccc(-c3ccc4c(c3)sc3ccc(-c5ccc(-c6nc(-c7ccccc7)nc(-c7ccccc7)n6)c6oc7ccccc7c56)cc34)cc2)cc1 ; c1ccc(-c2ccc(-c3ccc4sc5ccc(-c6ccc(-c7nc(-c8ccccc8)nc(-c8ccccc8)n7)c7oc8ccccc8c67)cc5c4c3)cc2)cc1]

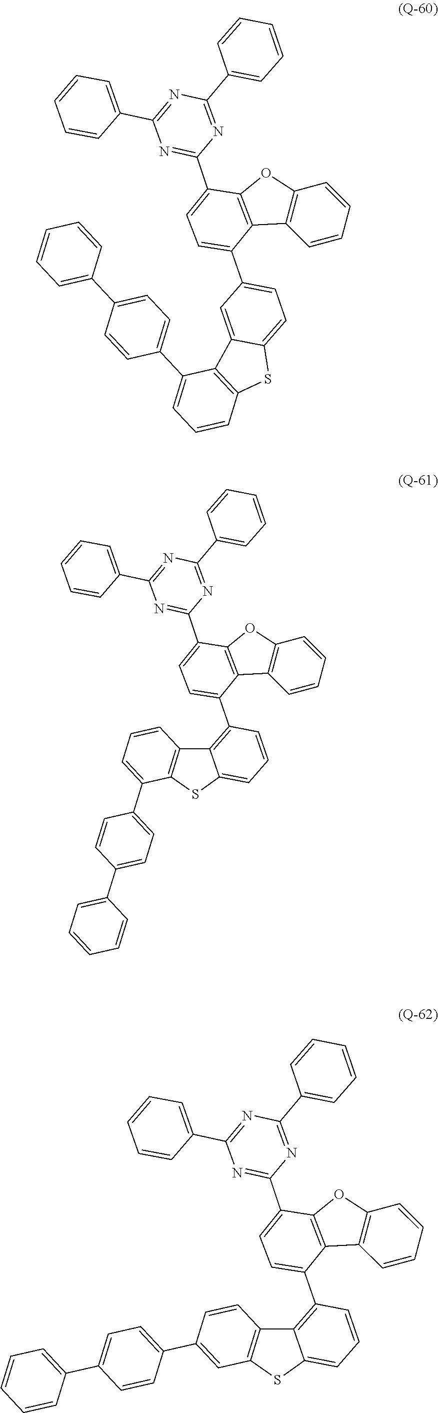 [SMILES: c1ccc(-c2ccc(-c3cccc4sc5ccc(-c6ccc(-c7nc(-c8ccccc8)nc(-c8ccccc8)n7)c7oc8ccccc8c67)cc5c34)cc2)cc1 ; c1ccc(-c2ccc(-c3cccc4c3sc3cccc(-c5ccc(-c6nc(-c7ccccc7)nc(-c7ccccc7)n6)c6oc7ccccc7c56)c34)cc2)cc1 ; c1ccc(-c2ccc(-c3ccc4c(c3)sc3cccc(-c5ccc(-c6nc(-c7ccccc7)nc(-c7ccccc7)n6)c6oc7ccccc7c56)c34)cc2)cc1]

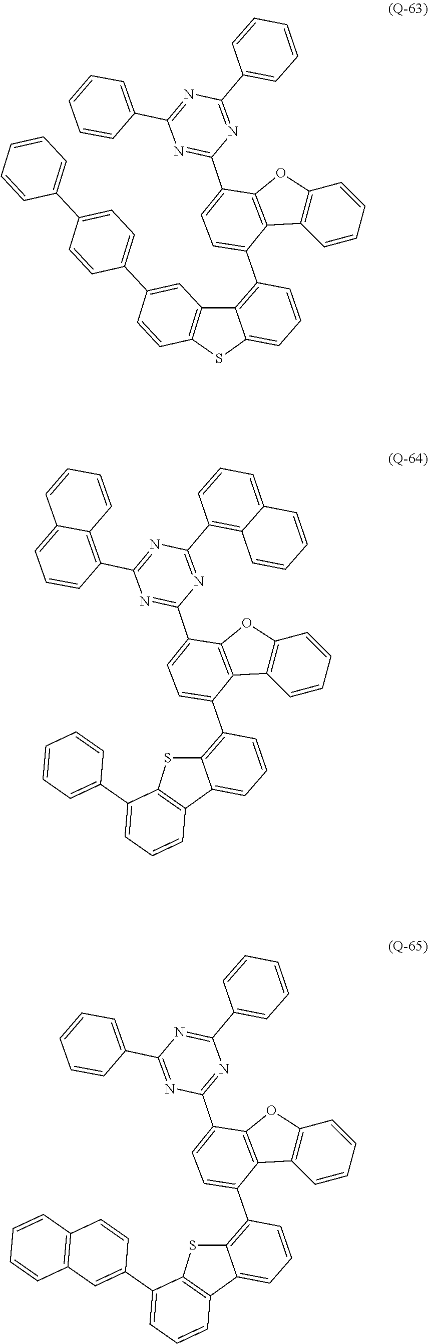 [SMILES: c1ccc(-c2ccc(-c3ccc4sc5cccc(-c6ccc(-c7nc(-c8ccccc8)nc(-c8ccccc8)n7)c7oc8ccccc8c67)c5c4c3)cc2)cc1 ; c1ccc(-c2cccc3c2sc2c(-c4ccc(-c5nc(-c6cccc7ccccc67)nc(-c6cccc7ccccc67)n5)c5oc6ccccc6c45)cccc23)cc1 ; c1ccc(-c2nc(-c3ccccc3)nc(-c3ccc(-c4cccc5c4sc4c(-c6ccc7ccccc7c6)cccc45)c4c3oc3ccccc34)n2)cc1]

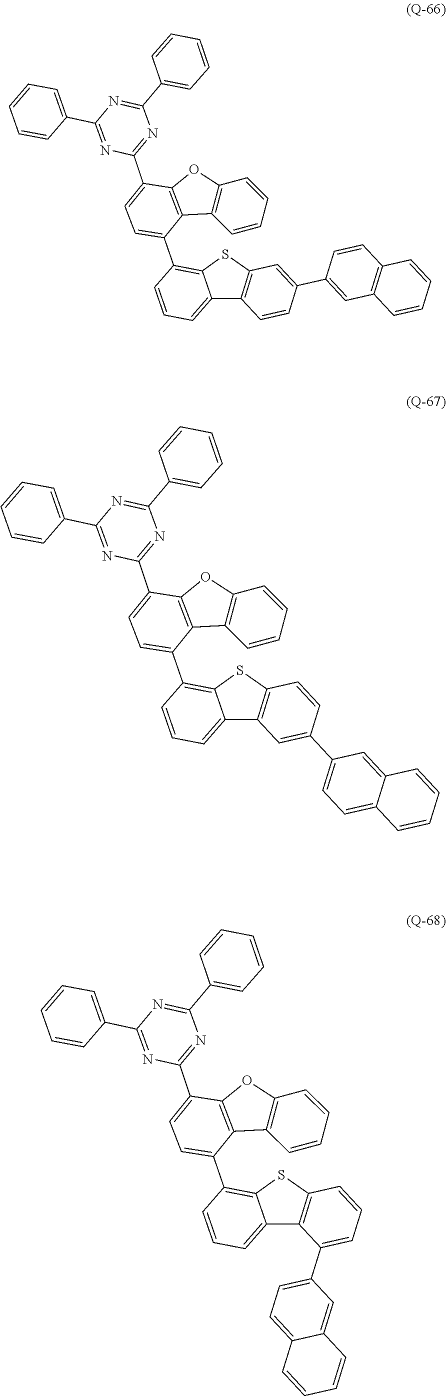 [SMILES: c1ccc(-c2nc(-c3ccccc3)nc(-c3ccc(-c4cccc5c4sc4cc(-c6ccc7ccccc7c6)ccc45)c4c3oc3ccccc34)n2)cc1 ; c1ccc(-c2nc(-c3ccccc3)nc(-c3ccc(-c4cccc5c4sc4ccc(-c6ccc7ccccc7c6)cc45)c4c3oc3ccccc34)n2)cc1 ; c1ccc(-c2nc(-c3ccccc3)nc(-c3ccc(-c4cccc5c4sc4cccc(-c6ccc7ccccc7c6)c45)c4c3oc3ccccc34)n2)cc1]

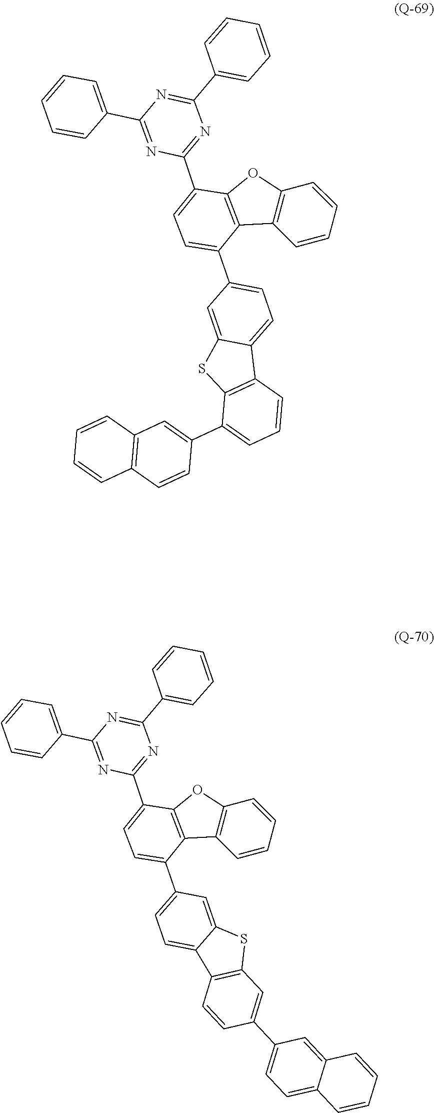 [SMILES: c1ccc(-c2nc(-c3ccccc3)nc(-c3ccc(-c4ccc5c(c4)sc4c(-c6ccc7ccccc7c6)cccc45)c4c3oc3ccccc34)n2)cc1 ; c1ccc(-c2nc(-c3ccccc3)nc(-c3ccc(-c4ccc5c(c4)sc4cc(-c6ccc7ccccc7c6)ccc45)c4c3oc3ccccc34)n2)cc1]

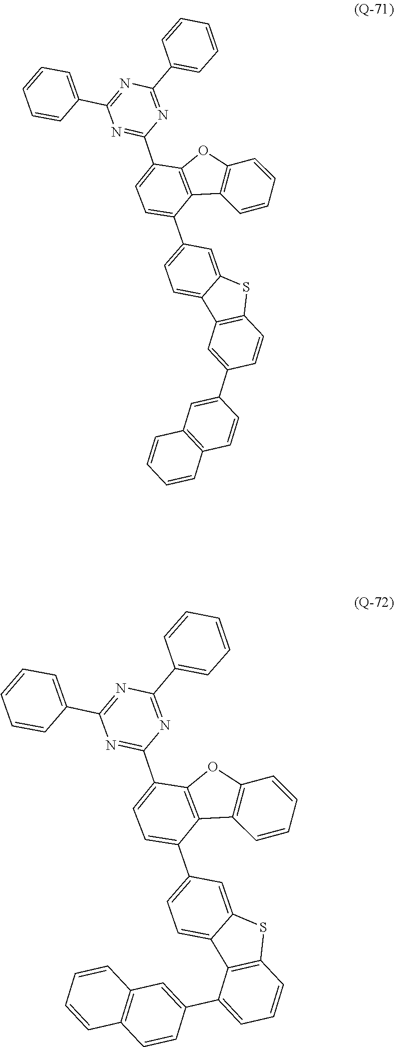 [SMILES: c1ccc(-c2nc(-c3ccccc3)nc(-c3ccc(-c4ccc5c(c4)sc4ccc(-c6ccc7ccccc7c6)cc45)c4c3oc3ccccc34)n2)cc1 ; c1ccc(-c2nc(-c3ccccc3)nc(-c3ccc(-c4ccc5c(c4)sc4cccc(-c6ccc7ccccc7c6)c45)c4c3oc3ccccc34)n2)cc1]

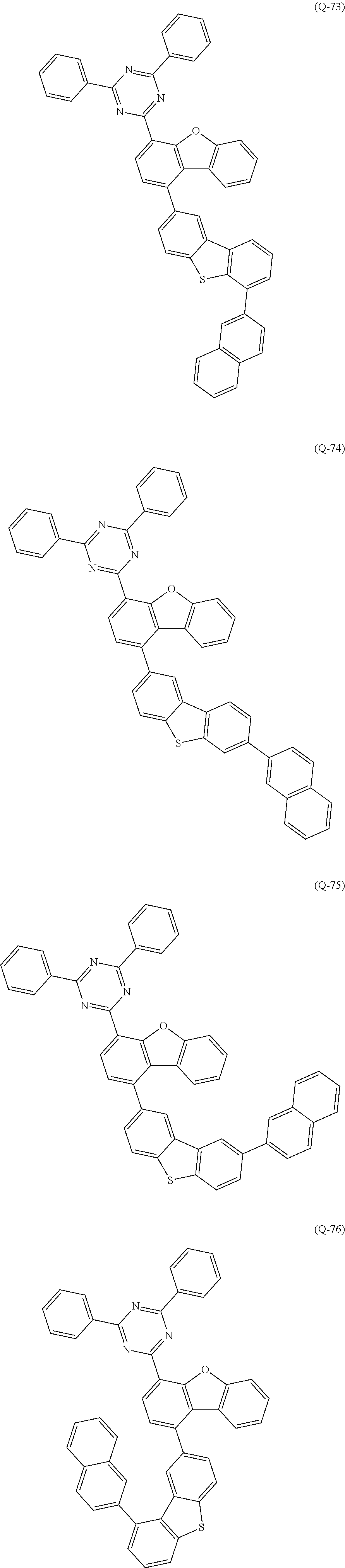 [SMILES: c1ccc(-c2nc(-c3ccccc3)nc(-c3ccc(-c4ccc5sc6c(-c7ccc8ccccc8c7)cccc6c5c4)c4c3oc3ccccc34)n2)cc1 ; c1ccc(-c2nc(-c3ccccc3)nc(-c3ccc(-c4ccc5sc6cc(-c7ccc8ccccc8c7)ccc6c5c4)c4c3oc3ccccc34)n2)cc1 ; c1ccc(-c2nc(-c3ccccc3)nc(-c3ccc(-c4ccc5sc6ccc(-c7ccc8ccccc8c7)cc6c5c4)c4c3oc3ccccc34)n2)cc1 ; c1ccc(-c2nc(-c3ccccc3)nc(-c3ccc(-c4ccc5sc6cccc(-c7ccc8ccccc8c7)c6c5c4)c4c3oc3ccccc34)n2)cc1]

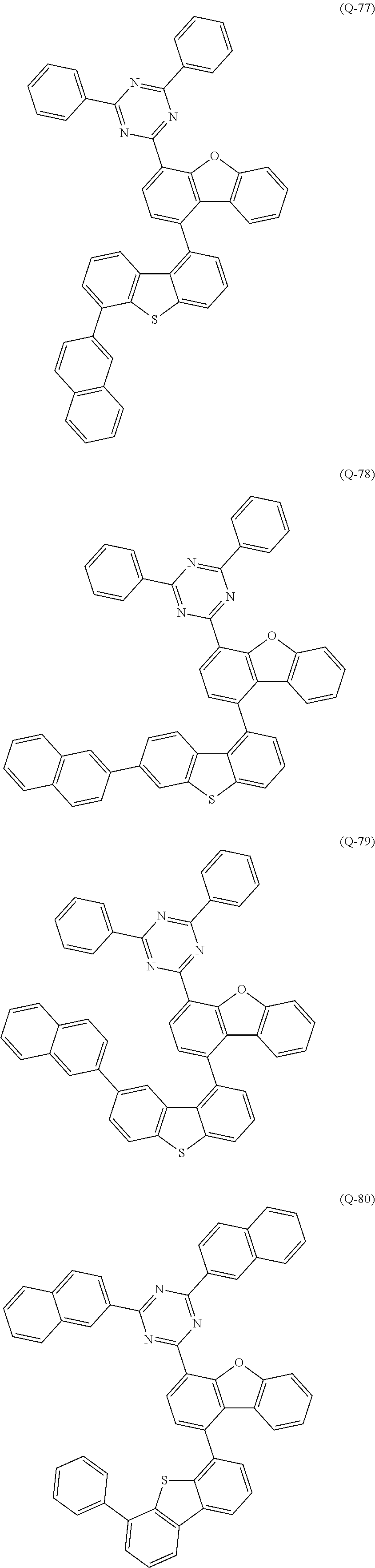 [SMILES: c1ccc(-c2nc(-c3ccccc3)nc(-c3ccc(-c4cccc5sc6c(-c7ccc8ccccc8c7)cccc6c45)c4c3oc3ccccc34)n2)cc1 ; c1ccc(-c2nc(-c3ccccc3)nc(-c3ccc(-c4cccc5sc6cc(-c7ccc8ccccc8c7)ccc6c45)c4c3oc3ccccc34)n2)cc1 ; c1ccc(-c2nc(-c3ccccc3)nc(-c3ccc(-c4cccc5sc6ccc(-c7ccc8ccccc8c7)cc6c45)c4c3oc3ccccc34)n2)cc1 ; c1ccc(-c2cccc3c2sc2c(-c4ccc(-c5nc(-c6ccc7ccccc7c6)nc(-c6ccc7ccccc7c6)n5)c5oc6ccccc6c45)cccc23)cc1]

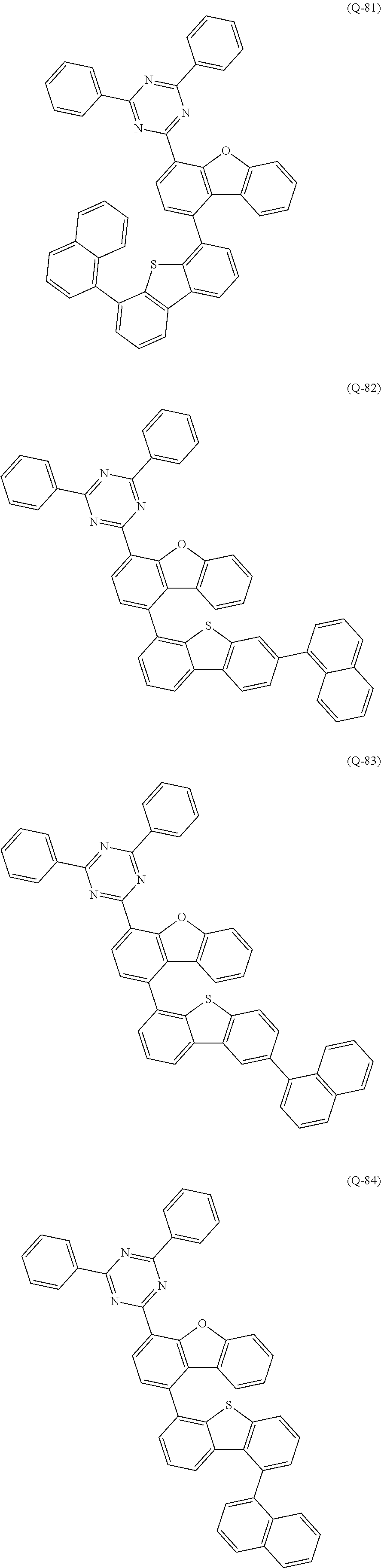 [SMILES: c1ccc(-c2nc(-c3ccccc3)nc(-c3ccc(-c4cccc5c4sc4c(-c6cccc7ccccc67)cccc45)c4c3oc3ccccc34)n2)cc1 ; c1ccc(-c2nc(-c3ccccc3)nc(-c3ccc(-c4cccc5c4sc4cc(-c6cccc7ccccc67)ccc45)c4c3oc3ccccc34)n2)cc1 ; c1ccc(-c2nc(-c3ccccc3)nc(-c3ccc(-c4cccc5c4sc4ccc(-c6cccc7ccccc67)cc45)c4c3oc3ccccc34)n2)cc1 ; c1ccc(-c2nc(-c3ccccc3)nc(-c3ccc(-c4cccc5c4sc4cccc(-c6cccc7ccccc67)c45)c4c3oc3ccccc34)n2)cc1]

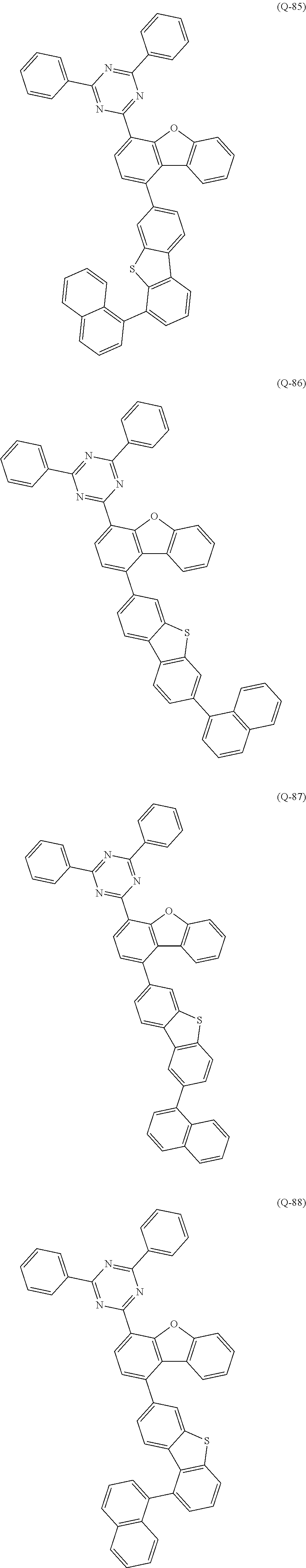 [SMILES: c1ccc(-c2nc(-c3ccccc3)nc(-c3ccc(-c4ccc5c(c4)sc4c(-c6cccc7ccccc67)cccc45)c4c3oc3ccccc34)n2)cc1 ; c1ccc(-c2nc(-c3ccccc3)nc(-c3ccc(-c4ccc5c(c4)sc4cc(-c6cccc7ccccc67)ccc45)c4c3oc3ccccc34)n2)cc1 ; c1ccc(-c2nc(-c3ccccc3)nc(-c3ccc(-c4ccc5c(c4)sc4ccc(-c6cccc7ccccc67)cc45)c4c3oc3ccccc34)n2)cc1 ; c1ccc(-c2nc(-c3ccccc3)nc(-c3ccc(-c4ccc5c(c4)sc4cccc(-c6cccc7ccccc67)c45)c4c3oc3ccccc34)n2)cc1]

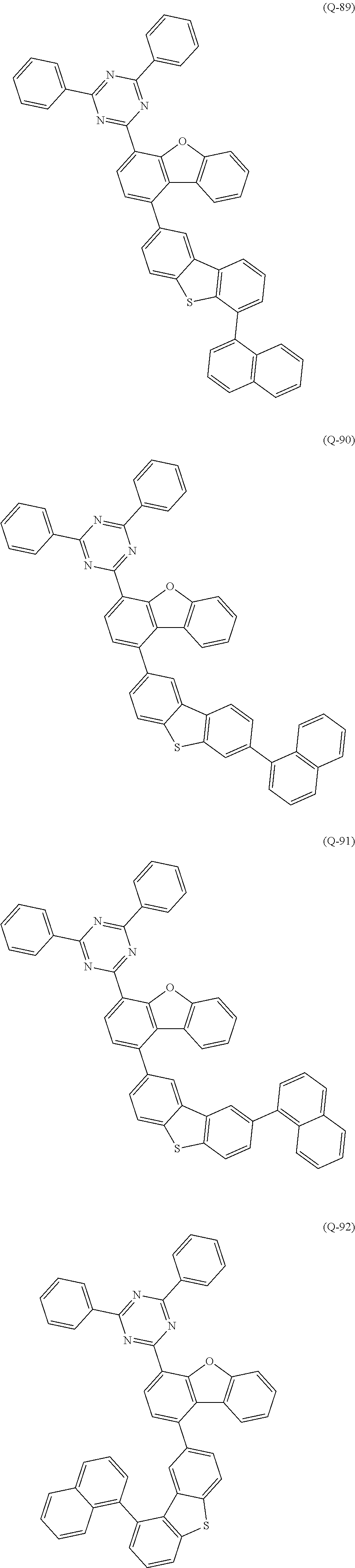 [SMILES: c1ccc(-c2nc(-c3ccccc3)nc(-c3ccc(-c4ccc5sc6c(-c7cccc8ccccc78)cccc6c5c4)c4c3oc3ccccc34)n2)cc1 ; c1ccc(-c2nc(-c3ccccc3)nc(-c3ccc(-c4ccc5sc6cc(-c7cccc8ccccc78)ccc6c5c4)c4c3oc3ccccc34)n2)cc1 ; c1ccc(-c2nc(-c3ccccc3)nc(-c3ccc(-c4ccc5sc6ccc(-c7cccc8ccccc78)cc6c5c4)c4c3oc3ccccc34)n2)cc1 ; c1ccc(-c2nc(-c3ccccc3)nc(-c3ccc(-c4ccc5sc6cccc(-c7cccc8ccccc78)c6c5c4)c4c3oc3ccccc34)n2)cc1]

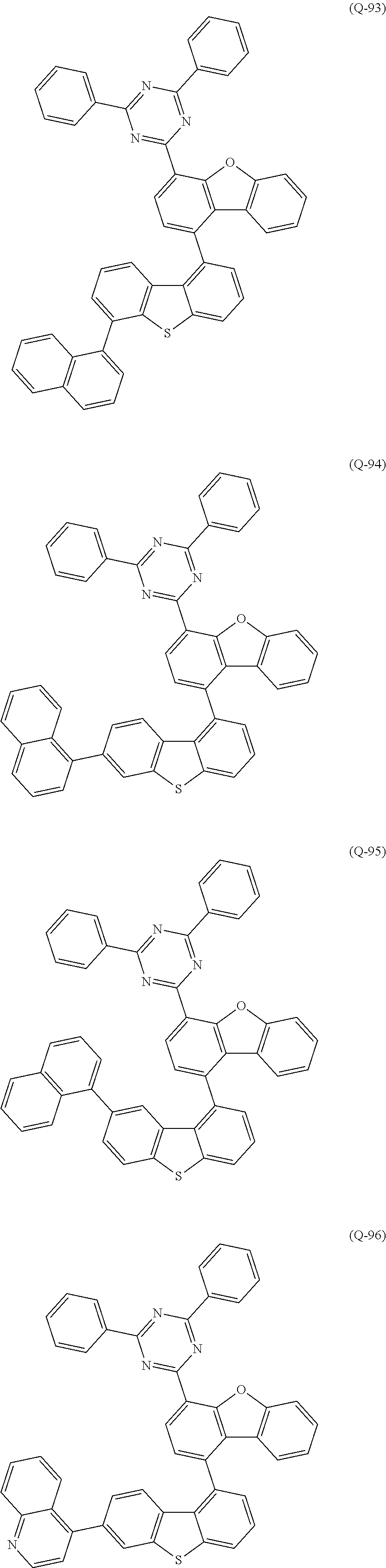 [SMILES: c1ccc(-c2nc(-c3ccccc3)nc(-c3ccc(-c4cccc5sc6c(-c7cccc8ccccc78)cccc6c45)c4c3oc3ccccc34)n2)cc1 ; c1ccc(-c2nc(-c3ccccc3)nc(-c3ccc(-c4cccc5sc6cc(-c7cccc8ccccc78)ccc6c45)c4c3oc3ccccc34)n2)cc1 ; c1ccc(-c2nc(-c3ccccc3)nc(-c3ccc(-c4cccc5sc6ccc(-c7cccc8ccccc78)cc6c45)c4c3oc3ccccc34)n2)cc1 ; c1ccc(-c2nc(-c3ccccc3)nc(-c3ccc(-c4cccc5sc6cc(-c7ccnc8ccccc78)ccc6c45)c4c3oc3ccccc34)n2)cc1]

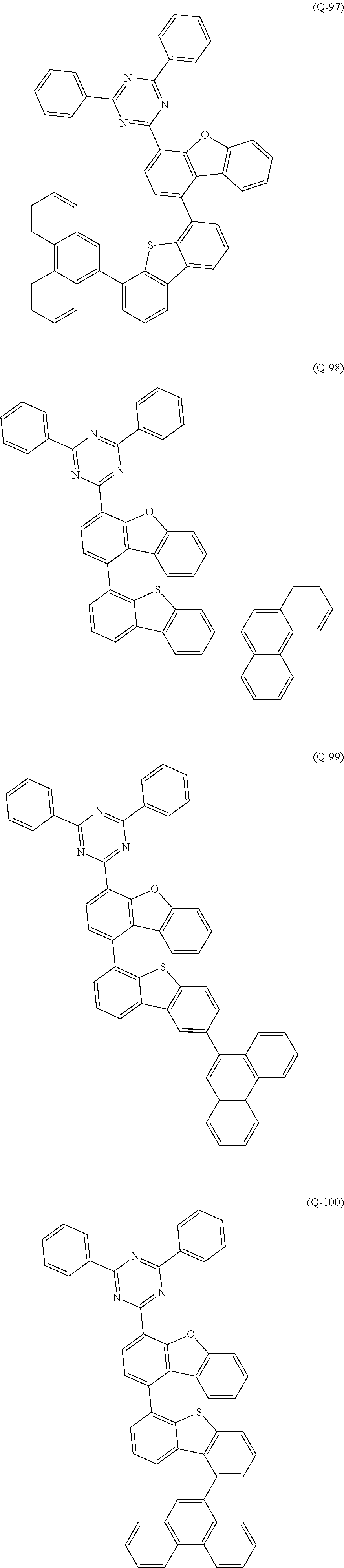 [SMILES: c1ccc(-c2nc(-c3ccccc3)nc(-c3ccc(-c4cccc5c4sc4c(-c6cc7ccccc7c7ccccc67)cccc45)c4c3oc3ccccc34)n2)cc1 ; c1ccc(-c2nc(-c3ccccc3)nc(-c3ccc(-c4cccc5c4sc4cc(-c6cc7ccccc7c7ccccc67)ccc45)c4c3oc3ccccc34)n2)cc1 ; c1ccc(-c2nc(-c3ccccc3)nc(-c3ccc(-c4cccc5c4sc4ccc(-c6cc7ccccc7c7ccccc67)cc45)c4c3oc3ccccc34)n2)cc1 ; c1ccc(-c2nc(-c3ccccc3)nc(-c3ccc(-c4cccc5c4sc4cccc(-c6cc7ccccc7c7ccccc67)c45)c4c3oc3ccccc34)n2)cc1]

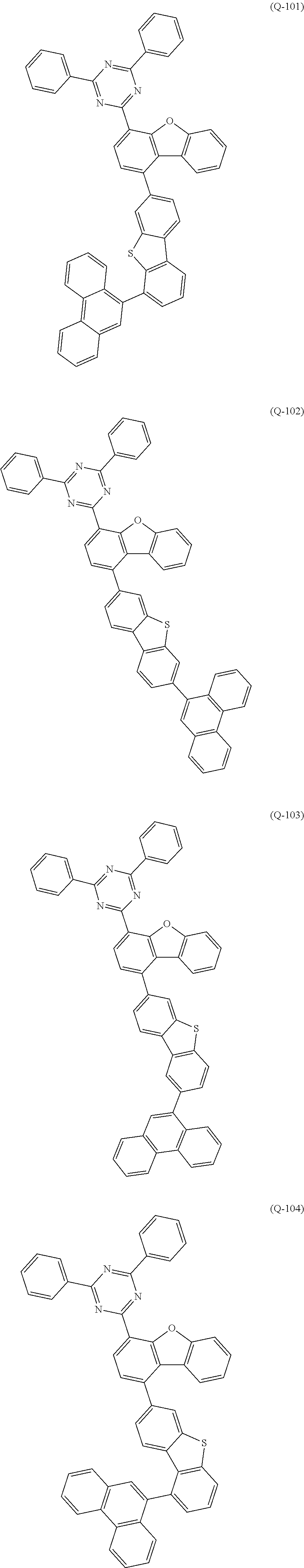 [SMILES: c1ccc(-c2nc(-c3ccccc3)nc(-c3ccc(-c4ccc5c(c4)sc4c(-c6cc7ccccc7c7ccccc67)cccc45)c4c3oc3ccccc34)n2)cc1 ; c1ccc(-c2nc(-c3ccccc3)nc(-c3ccc(-c4ccc5c(c4)sc4cc(-c6cc7ccccc7c7ccccc67)ccc45)c4c3oc3ccccc34)n2)cc1 ; c1ccc(-c2nc(-c3ccccc3)nc(-c3ccc(-c4ccc5c(c4)sc4ccc(-c6cc7ccccc7c7ccccc67)cc45)c4c3oc3ccccc34)n2)cc1 ; c1ccc(-c2nc(-c3ccccc3)nc(-c3ccc(-c4ccc5c(c4)sc4cccc(-c6cc7ccccc7c7ccccc67)c45)c4c3oc3ccccc34)n2)cc1]

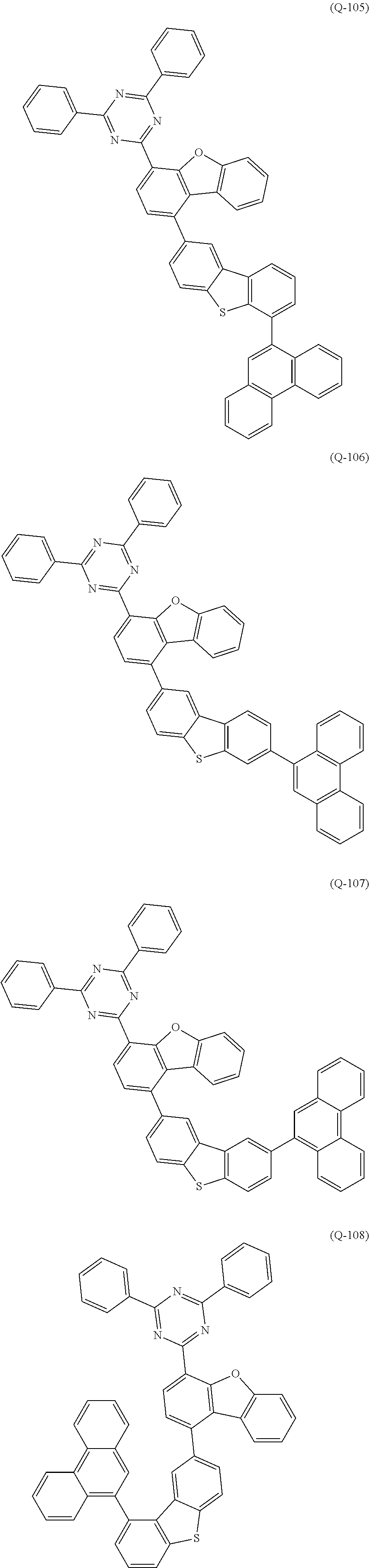 [SMILES: c1ccc(-c2nc(-c3ccccc3)nc(-c3ccc(-c4ccc5sc6c(-c7cc8ccccc8c8ccccc78)cccc6c5c4)c4c3oc3ccccc34)n2)cc1 ; c1ccc(-c2nc(-c3ccccc3)nc(-c3ccc(-c4ccc5sc6cc(-c7cc8ccccc8c8ccccc78)ccc6c5c4)c4c3oc3ccccc34)n2)cc1 ; c1ccc(-c2nc(-c3ccccc3)nc(-c3ccc(-c4ccc5sc6ccc(-c7cc8ccccc8c8ccccc78)cc6c5c4)c4c3oc3ccccc34)n2)cc1 ; c1ccc(-c2nc(-c3ccccc3)nc(-c3ccc(-c4ccc5sc6cccc(-c7cc8ccccc8c8ccccc78)c6c5c4)c4c3oc3ccccc34)n2)cc1]

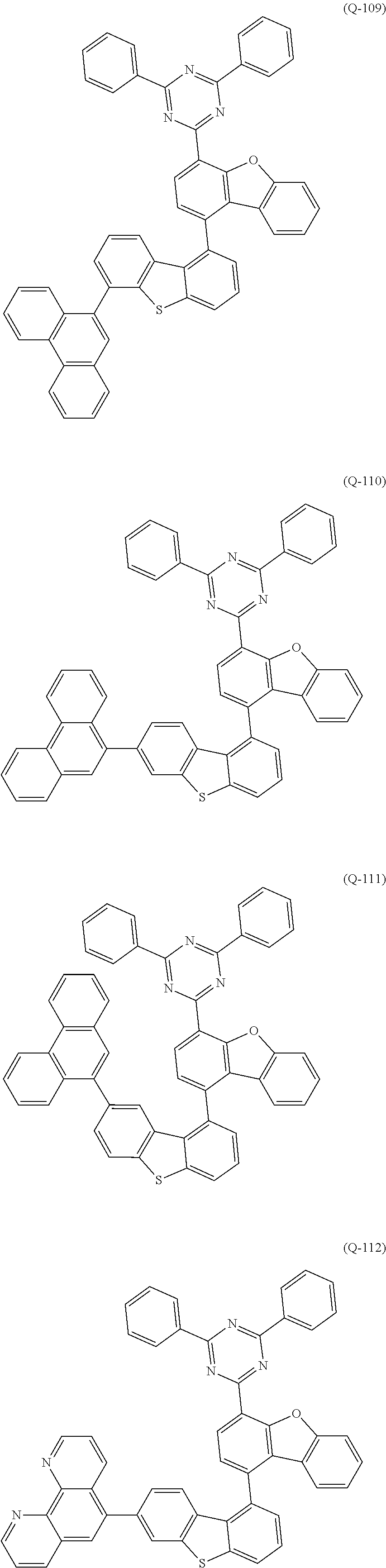 [SMILES: c1ccc(-c2nc(-c3ccccc3)nc(-c3ccc(-c4cccc5sc6c(-c7cc8ccccc8c8ccccc78)cccc6c45)c4c3oc3ccccc34)n2)cc1 ; c1ccc(-c2nc(-c3ccccc3)nc(-c3ccc(-c4cccc5sc6cc(-c7cc8ccccc8c8ccccc78)ccc6c45)c4c3oc3ccccc34)n2)cc1 ; c1ccc(-c2nc(-c3ccccc3)nc(-c3ccc(-c4cccc5sc6ccc(-c7cc8ccccc8c8ccccc78)cc6c45)c4c3oc3ccccc34)n2)cc1 ; c1ccc(-c2nc(-c3ccccc3)nc(-c3ccc(-c4cccc5sc6cc(-c7cc8cccnc8c8ncccc78)ccc6c45)c4c3oc3ccccc34)n2)cc1]

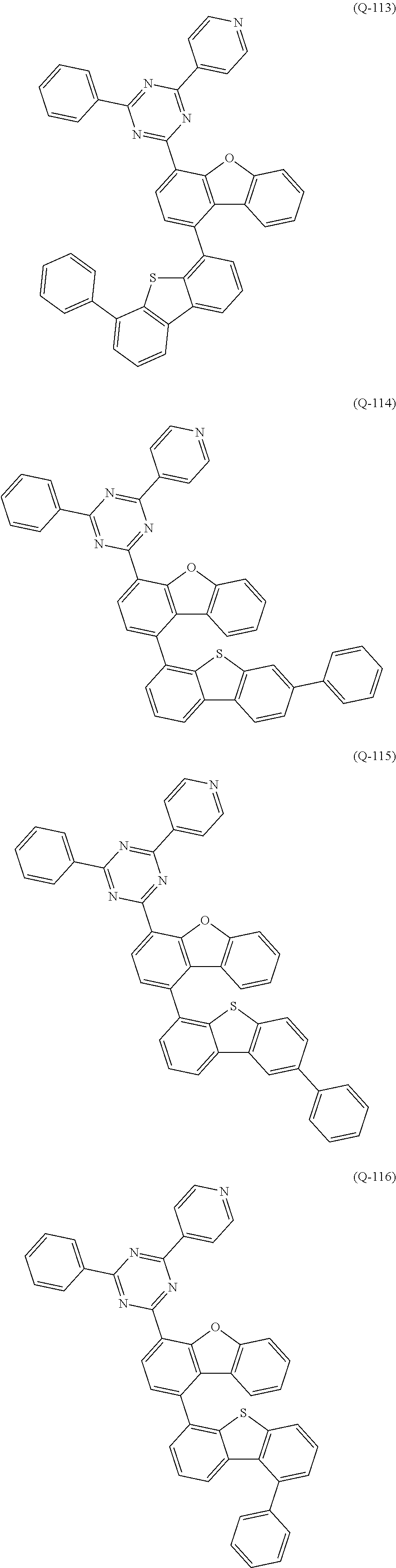 [SMILES: c1ccc(-c2nc(-c3ccncc3)nc(-c3ccc(-c4cccc5c4sc4c(-c6ccccc6)cccc45)c4c3oc3ccccc34)n2)cc1 ; c1ccc(-c2ccc3c(c2)sc2c(-c4ccc(-c5nc(-c6ccccc6)nc(-c6ccncc6)n5)c5oc6ccccc6c45)cccc23)cc1 ; c1ccc(-c2ccc3sc4c(-c5ccc(-c6nc(-c7ccccc7)nc(-c7ccncc7)n6)c6oc7ccccc7c56)cccc4c3c2)cc1 ; c1ccc(-c2nc(-c3ccncc3)nc(-c3ccc(-c4cccc5c4sc4cccc(-c6ccccc6)c45)c4c3oc3ccccc34)n2)cc1]

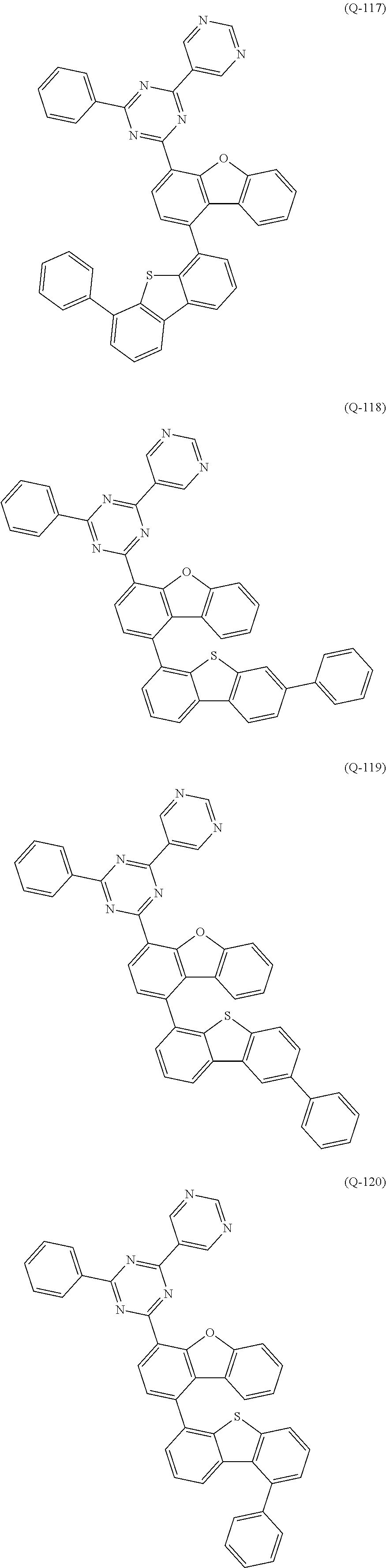 [SMILES: c1ccc(-c2nc(-c3cncnc3)nc(-c3ccc(-c4cccc5c4sc4c(-c6ccccc6)cccc45)c4c3oc3ccccc34)n2)cc1 ; c1ccc(-c2ccc3c(c2)sc2c(-c4ccc(-c5nc(-c6ccccc6)nc(-c6cncnc6)n5)c5oc6ccccc6c45)cccc23)cc1 ; c1ccc(-c2ccc3sc4c(-c5ccc(-c6nc(-c7ccccc7)nc(-c7cncnc7)n6)c6oc7ccccc7c56)cccc4c3c2)cc1 ; c1ccc(-c2nc(-c3cncnc3)nc(-c3ccc(-c4cccc5c4sc4cccc(-c6ccccc6)c45)c4c3oc3ccccc34)n2)cc1]

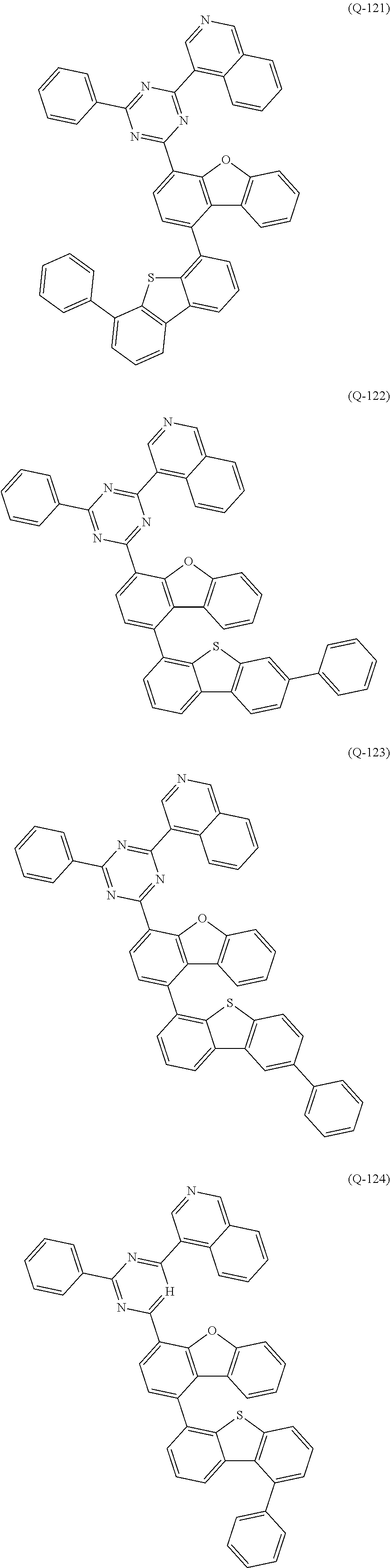

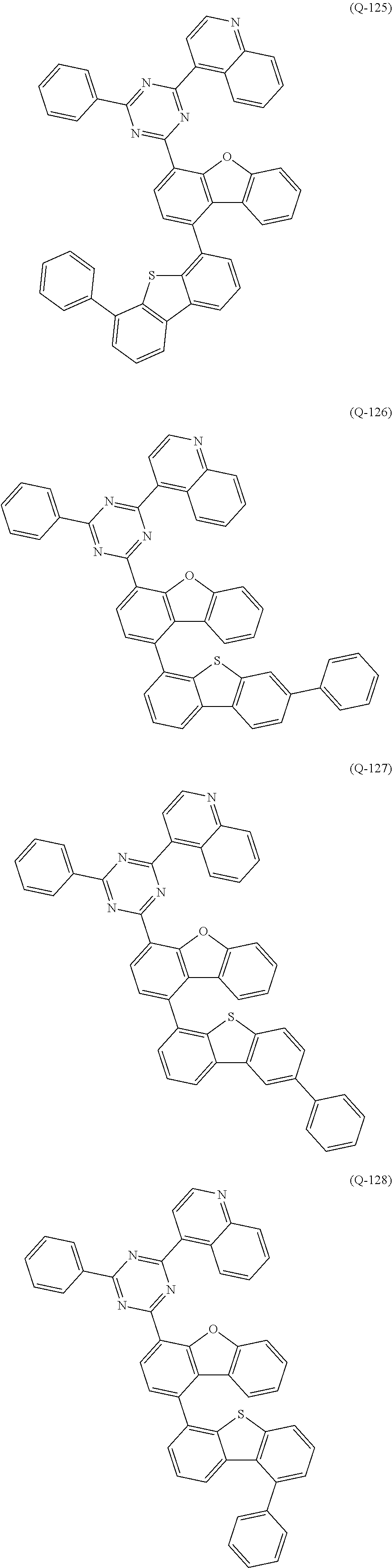 [SMILES: c1ccc(-c2nc(-c3ccnc4ccccc34)nc(-c3ccc(-c4cccc5c4sc4c(-c6ccccc6)cccc45)c4c3oc3ccccc34)n2)cc1 ; c1ccc(-c2ccc3c(c2)sc2c(-c4ccc(-c5nc(-c6ccccc6)nc(-c6ccnc7ccccc67)n5)c5oc6ccccc6c45)cccc23)cc1 ; c1ccc(-c2ccc3sc4c(-c5ccc(-c6nc(-c7ccccc7)nc(-c7ccnc8ccccc78)n6)c6oc7ccccc7c56)cccc4c3c2)cc1 ; c1ccc(-c2nc(-c3ccnc4ccccc34)nc(-c3ccc(-c4cccc5c4sc4cccc(-c6ccccc6)c45)c4c3oc3ccccc34)n2)cc1]

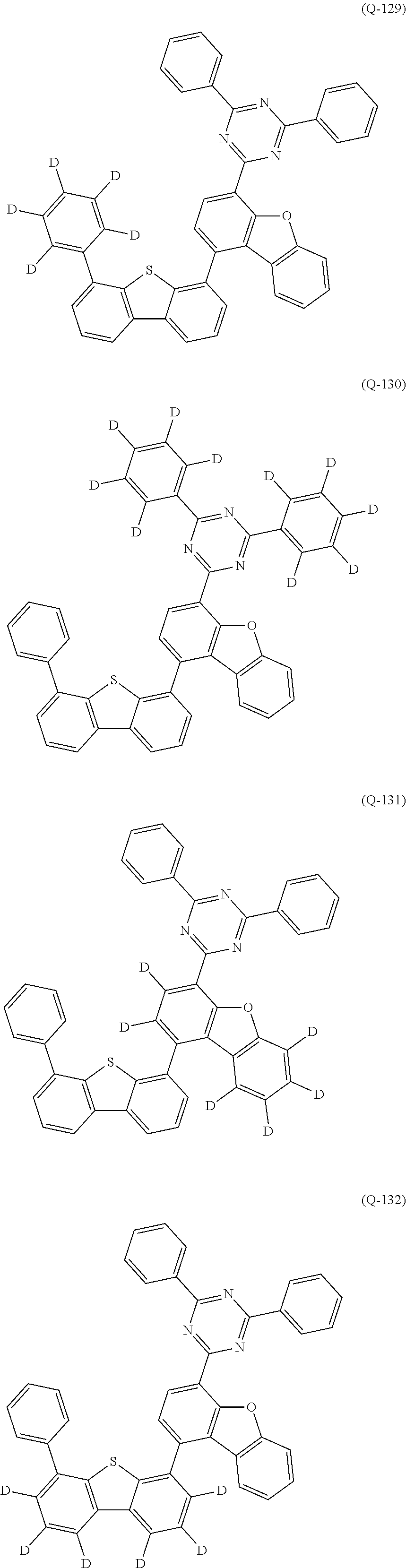 [SMILES: [2H]c1c([2H])c([2H])c(-c2cccc3c2sc2c(-c4ccc(-c5nc(-c6ccccc6)nc(-c6ccccc6)n5)c5oc6ccccc6c45)cccc23)c([2H])c1[2H] ; [2H]c1c([2H])c([2H])c(-c2nc(-c3c([2H])c([2H])c([2H])c([2H])c3[2H])nc(-c3ccc(-c4cccc5c4sc4c(-c6ccccc6)cccc45)c4c3oc3ccccc34)n2)c([2H])c1[2H] ; [2H]c1c([2H])c([2H])c2c(oc3c(-c4nc(-c5ccccc5)nc(-c5ccccc5)n4)c([2H])c([2H])c(-c4cccc5c4sc4c(-c6ccccc6)cccc45)c32)c1[2H] ; [2H]c1c([2H])c([2H])c2c(sc3c(-c4ccc(-c5nc(-c6ccccc6)nc(-c6ccccc6)n5)c5oc6ccccc6c45)c([2H])c([2H])c([2H])c32)c1-c1ccccc1]

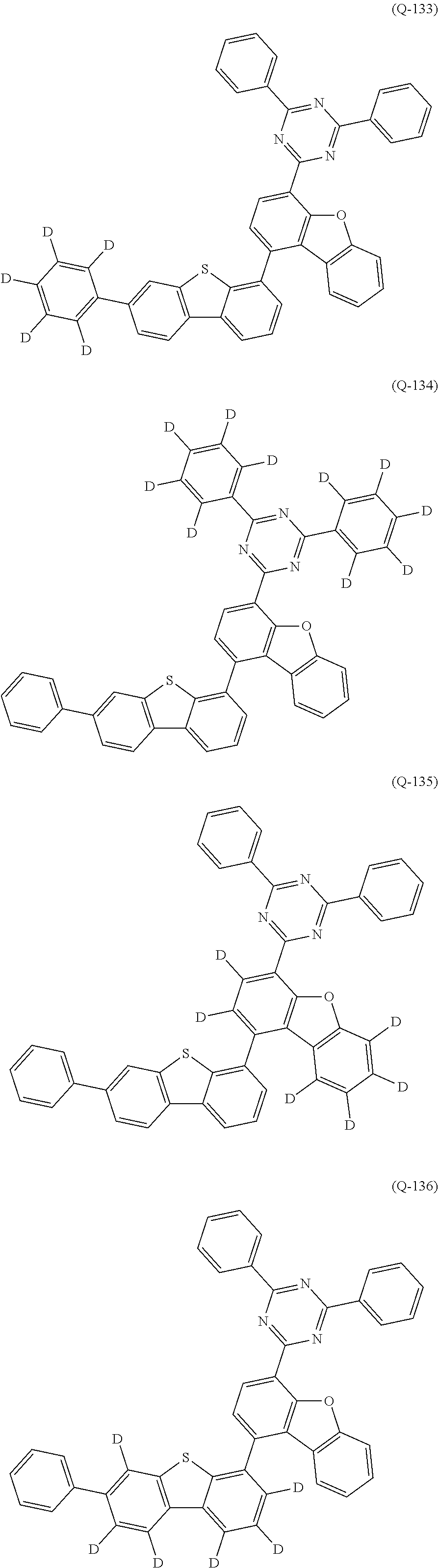 [SMILES: [2H]c1c([2H])c([2H])c(-c2ccc3c(c2)sc2c(-c4ccc(-c5nc(-c6ccccc6)nc(-c6ccccc6)n5)c5oc6ccccc6c45)cccc23)c([2H])c1[2H] ; [2H]c1c([2H])c([2H])c(-c2nc(-c3c([2H])c([2H])c([2H])c([2H])c3[2H])nc(-c3ccc(-c4cccc5c4sc4cc(-c6ccccc6)ccc45)c4c3oc3ccccc34)n2)c([2H])c1[2H] ; [2H]c1c([2H])c([2H])c2c(oc3c(-c4nc(-c5ccccc5)nc(-c5ccccc5)n4)c([2H])c([2H])c(-c4cccc5c4sc4cc(-c6ccccc6)ccc45)c32)c1[2H] ; [2H]c1c([2H])c([2H])c2c(sc3c([2H])c(-c4ccccc4)c([2H])c([2H])c32)c1-c1ccc(-c2nc(-c3ccccc3)nc(-c3ccccc3)n2)c2oc3ccccc3c12]

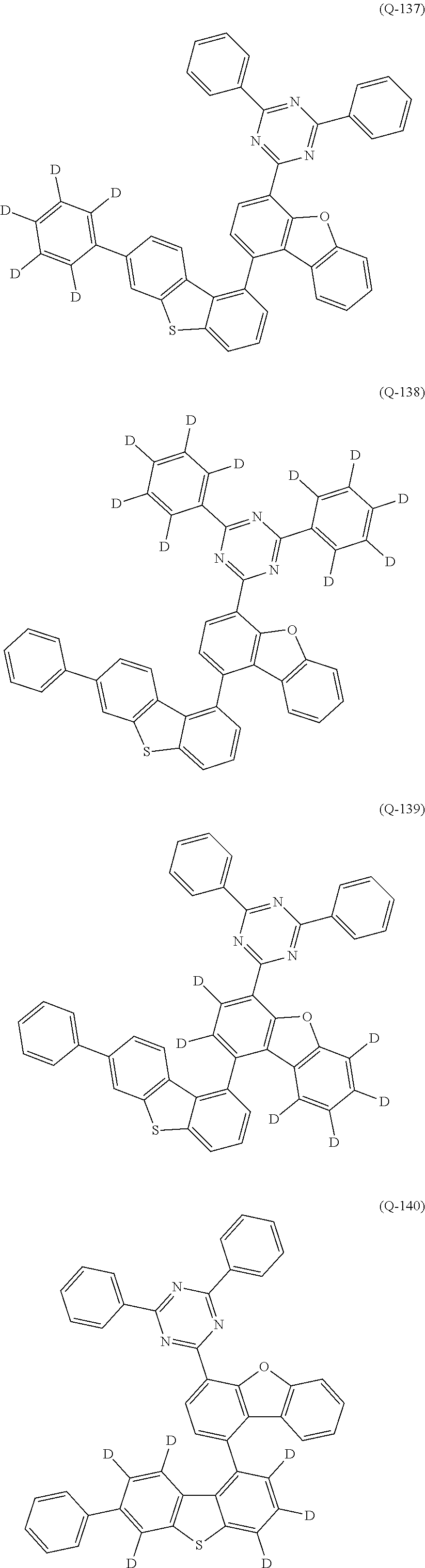 [SMILES: [2H]c1c([2H])c([2H])c(-c2ccc3c(c2)sc2cccc(-c4ccc(-c5nc(-c6ccccc6)nc(-c6ccccc6)n5)c5oc6ccccc6c45)c23)c([2H])c1[2H] ; [2H]c1c([2H])c([2H])c(-c2nc(-c3c([2H])c([2H])c([2H])c([2H])c3[2H])nc(-c3ccc(-c4cccc5sc6cc(-c7ccccc7)ccc6c45)c4c3oc3ccccc34)n2)c([2H])c1[2H] ; [2H]c1c([2H])c([2H])c2c(oc3c(-c4nc(-c5ccccc5)nc(-c5ccccc5)n4)c([2H])c([2H])c(-c4cccc5sc6cc(-c7ccccc7)ccc6c45)c32)c1[2H] ; [2H]c1c([2H])c(-c2ccc(-c3nc(-c4ccccc4)nc(-c4ccccc4)n3)c3oc4ccccc4c23)c2c(sc3c([2H])c(-c4ccccc4)c([2H])c([2H])c32)c1[2H]]

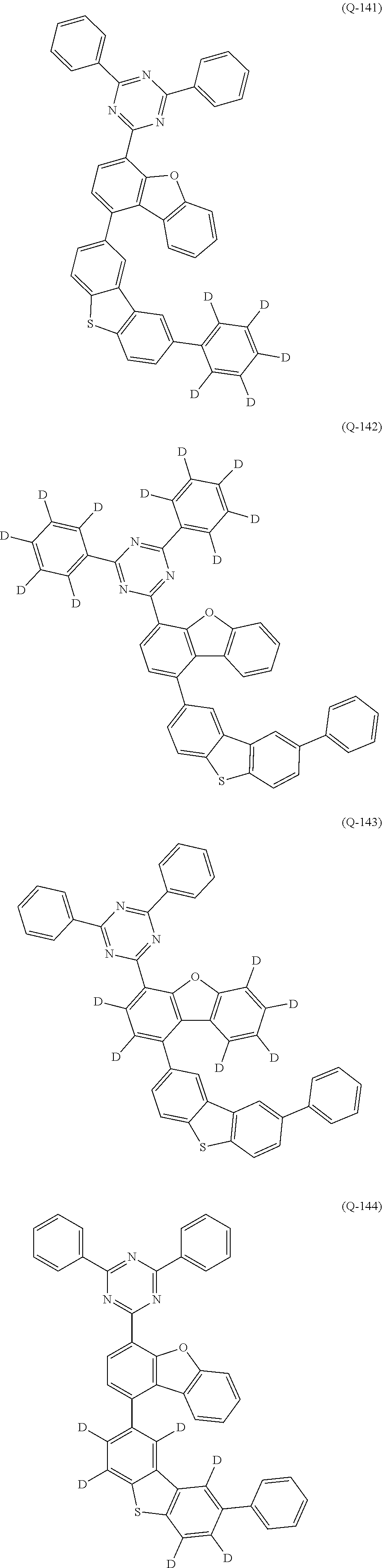 [SMILES: [2H]c1c([2H])c([2H])c(-c2ccc3sc4ccc(-c5ccc(-c6nc(-c7ccccc7)nc(-c7ccccc7)n6)c6oc7ccccc7c56)cc4c3c2)c([2H])c1[2H] ; [2H]c1c([2H])c([2H])c(-c2nc(-c3c([2H])c([2H])c([2H])c([2H])c3[2H])nc(-c3ccc(-c4ccc5sc6ccc(-c7ccccc7)cc6c5c4)c4c3oc3ccccc34)n2)c([2H])c1[2H] ; [2H]c1c([2H])c([2H])c2c(oc3c(-c4nc(-c5ccccc5)nc(-c5ccccc5)n4)c([2H])c([2H])c(-c4ccc5sc6ccc(-c7ccccc7)cc6c5c4)c32)c1[2H] ; [2H]c1c(-c2ccccc2)c([2H])c2c(sc3c([2H])c([2H])c(-c4ccc(-c5nc(-c6ccccc6)nc(-c6ccccc6)n5)c5oc6ccccc6c45)c([2H])c32)c1[2H]]

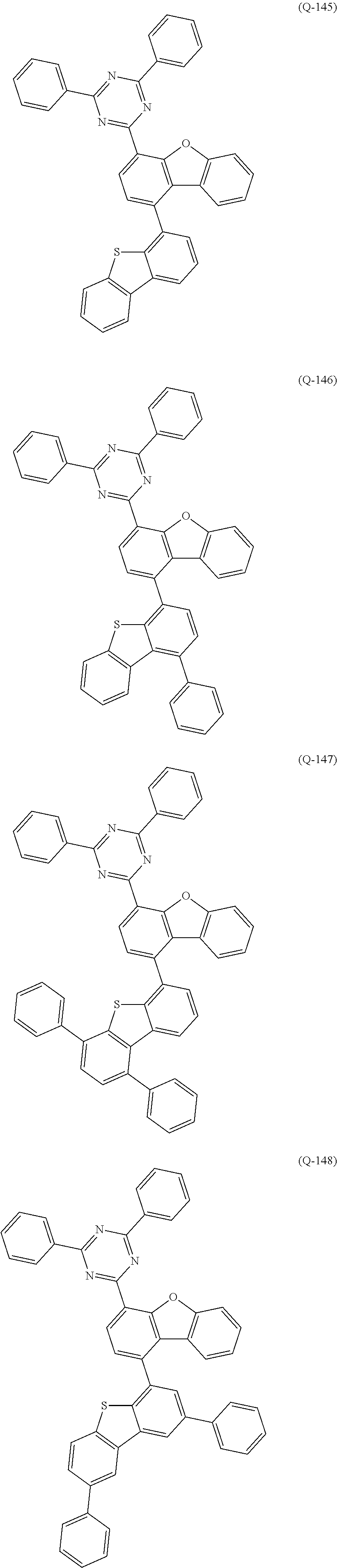 [SMILES: c1ccc(-c2nc(-c3ccccc3)nc(-c3ccc(-c4cccc5c4sc4ccccc45)c4c3oc3ccccc34)n2)cc1 ; c1ccc(-c2nc(-c3ccccc3)nc(-c3ccc(-c4ccc(-c5ccccc5)c5c4sc4ccccc45)c4c3oc3ccccc34)n2)cc1 ; c1ccc(-c2nc(-c3ccccc3)nc(-c3ccc(-c4cccc5c4sc4c(-c6ccccc6)ccc(-c6ccccc6)c45)c4c3oc3ccccc34)n2)cc1 ; c1ccc(-c2ccc3sc4c(-c5ccc(-c6nc(-c7ccccc7)nc(-c7ccccc7)n6)c6oc7ccccc7c56)cc(-c5ccccc5)cc4c3c2)cc1]

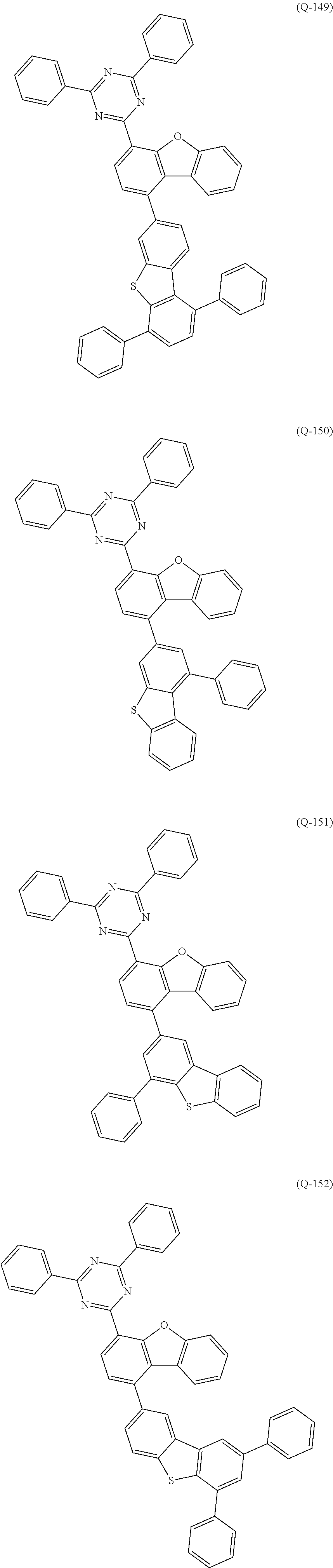 [SMILES: c1ccc(-c2nc(-c3ccccc3)nc(-c3ccc(-c4ccc5c(c4)sc4c(-c6ccccc6)ccc(-c6ccccc6)c45)c4c3oc3ccccc34)n2)cc1 ; c1ccc(-c2nc(-c3ccccc3)nc(-c3ccc(-c4cc(-c5ccccc5)c5c(c4)sc4ccccc45)c4c3oc3ccccc34)n2)cc1 ; c1ccc(-c2nc(-c3ccccc3)nc(-c3ccc(-c4cc(-c5ccccc5)c5sc6ccccc6c5c4)c4c3oc3ccccc34)n2)cc1 ; c1ccc(-c2cc(-c3ccccc3)c3sc4ccc(-c5ccc(-c6nc(-c7ccccc7)nc(-c7ccccc7)n6)c6oc7ccccc7c56)cc4c3c2)cc1]

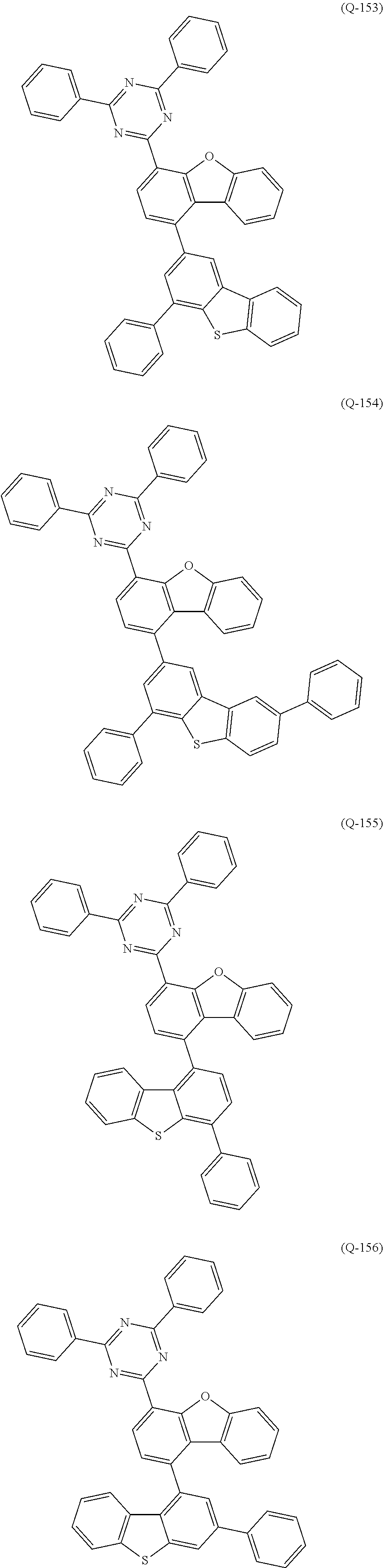 [SMILES: c1ccc(-c2nc(-c3ccccc3)nc(-c3ccc(-c4cc(-c5ccccc5)c5sc6ccccc6c5c4)c4c3oc3ccccc34)n2)cc1 ; c1ccc(-c2ccc3sc4c(-c5ccccc5)cc(-c5ccc(-c6nc(-c7ccccc7)nc(-c7ccccc7)n6)c6oc7ccccc7c56)cc4c3c2)cc1 ; c1ccc(-c2nc(-c3ccccc3)nc(-c3ccc(-c4ccc(-c5ccccc5)c5sc6ccccc6c45)c4c3oc3ccccc34)n2)cc1 ; c1ccc(-c2cc(-c3ccc(-c4nc(-c5ccccc5)nc(-c5ccccc5)n4)c4oc5ccccc5c34)c3c(c2)sc2ccccc23)cc1]

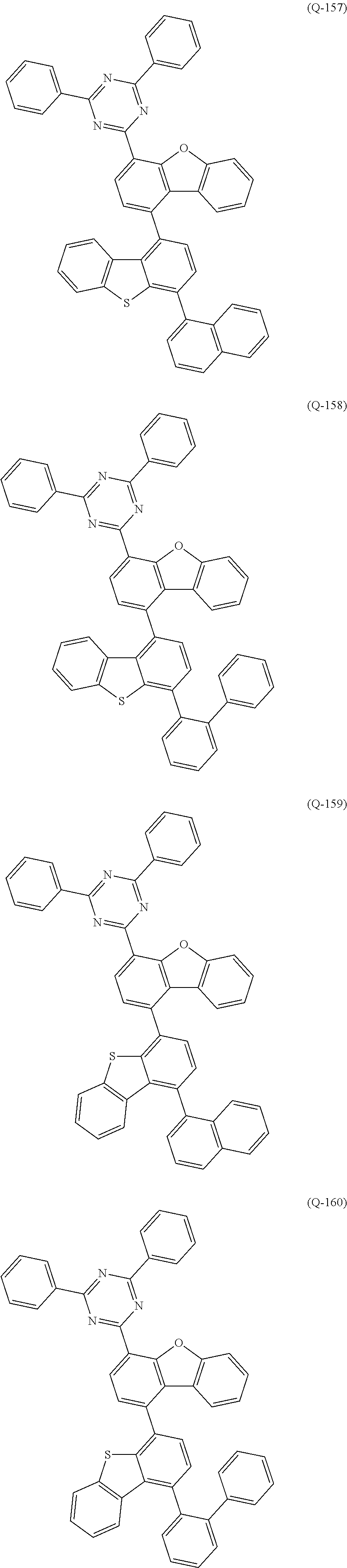 [SMILES: c1ccc(-c2nc(-c3ccccc3)nc(-c3ccc(-c4ccc(-c5cccc6ccccc56)c5sc6ccccc6c45)c4c3oc3ccccc34)n2)cc1 ; c1ccc(-c2nc(-c3ccccc3)nc(-c3ccc(-c4ccc(-c5ccccc5-c5ccccc5)c5sc6ccccc6c45)c4c3oc3ccccc34)n2)cc1 ; c1ccc(-c2nc(-c3ccccc3)nc(-c3ccc(-c4ccc(-c5cccc6ccccc56)c5c4sc4ccccc45)c4c3oc3ccccc34)n2)cc1 ; c1ccc(-c2nc(-c3ccccc3)nc(-c3ccc(-c4ccc(-c5ccccc5-c5ccccc5)c5c4sc4ccccc45)c4c3oc3ccccc34)n2)cc1]

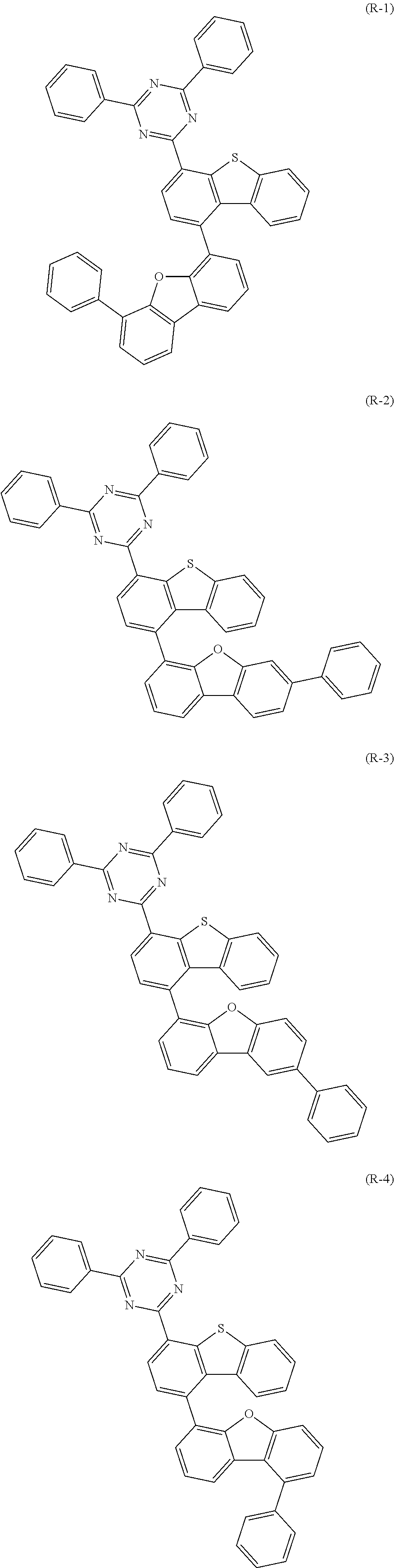 [SMILES: c1ccc(-c2nc(-c3ccccc3)nc(-c3ccc(-c4cccc5c4oc4c(-c6ccccc6)cccc45)c4c3sc3ccccc34)n2)cc1 ; c1ccc(-c2ccc3c(c2)oc2c(-c4ccc(-c5nc(-c6ccccc6)nc(-c6ccccc6)n5)c5sc6ccccc6c45)cccc23)cc1 ; c1ccc(-c2ccc3oc4c(-c5ccc(-c6nc(-c7ccccc7)nc(-c7ccccc7)n6)c6sc7ccccc7c56)cccc4c3c2)cc1 ; c1ccc(-c2nc(-c3ccccc3)nc(-c3ccc(-c4cccc5c4oc4cccc(-c6ccccc6)c45)c4c3sc3ccccc34)n2)cc1]

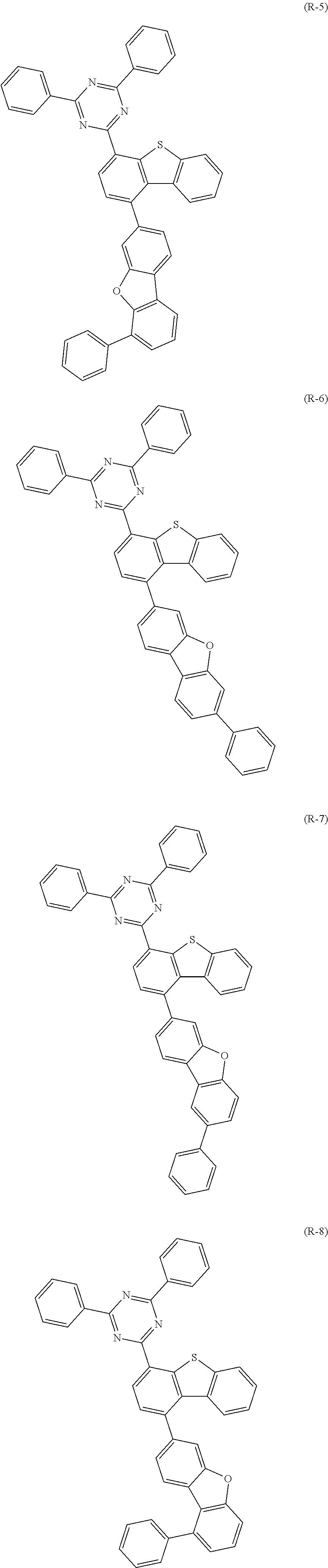

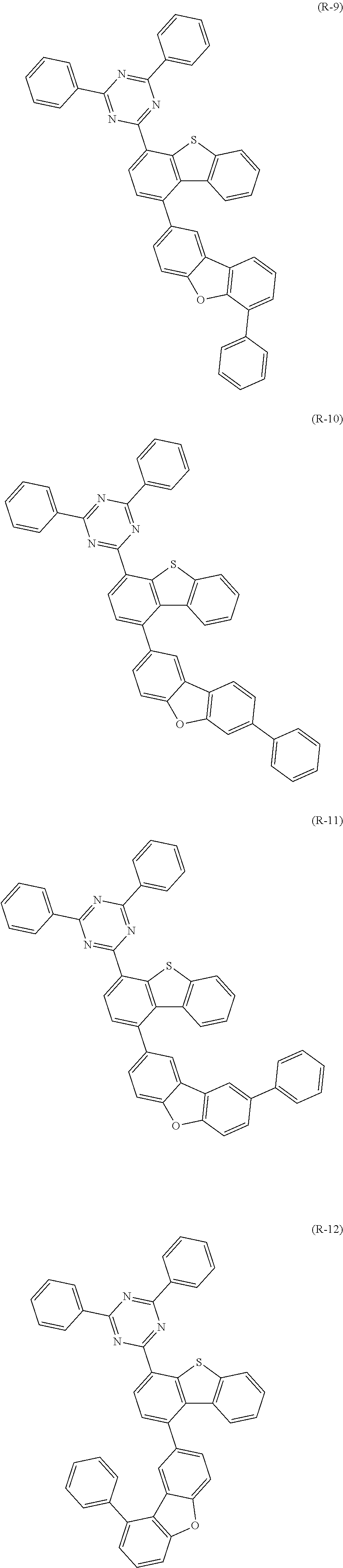 [SMILES: c1ccc(-c2nc(-c3ccccc3)nc(-c3ccc(-c4ccc5oc6c(-c7ccccc7)cccc6c5c4)c4c3sc3ccccc34)n2)cc1 ; c1ccc(-c2ccc3c(c2)oc2ccc(-c4ccc(-c5nc(-c6ccccc6)nc(-c6ccccc6)n5)c5sc6ccccc6c45)cc23)cc1 ; c1ccc(-c2ccc3oc4ccc(-c5ccc(-c6nc(-c7ccccc7)nc(-c7ccccc7)n6)c6sc7ccccc7c56)cc4c3c2)cc1 ; c1ccc(-c2nc(-c3ccccc3)nc(-c3ccc(-c4ccc5oc6cccc(-c7ccccc7)c6c5c4)c4c3sc3ccccc34)n2)cc1]

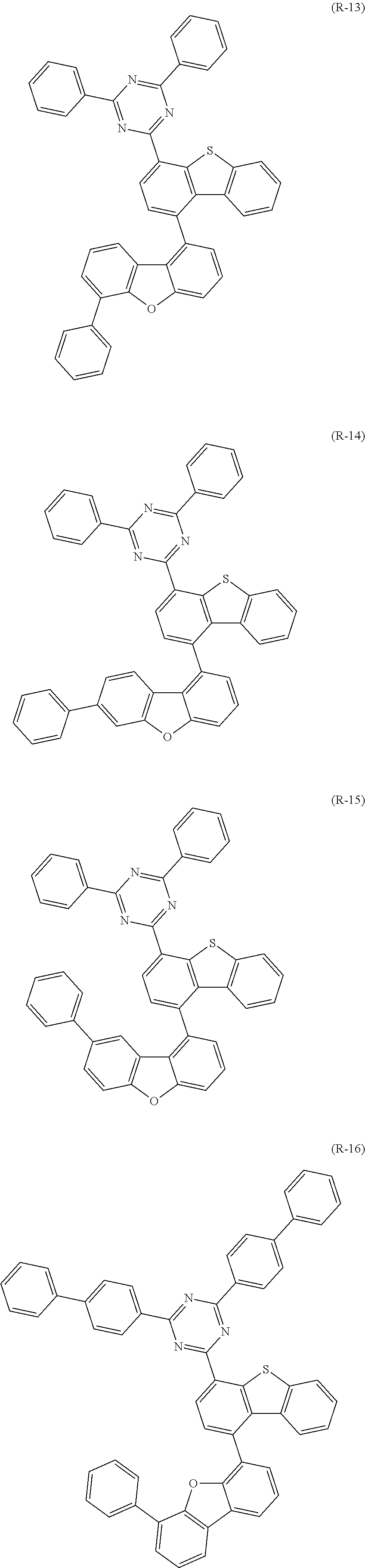 [SMILES: c1ccc(-c2nc(-c3ccccc3)nc(-c3ccc(-c4cccc5oc6c(-c7ccccc7)cccc6c45)c4c3sc3ccccc34)n2)cc1 ; c1ccc(-c2ccc3c(c2)oc2cccc(-c4ccc(-c5nc(-c6ccccc6)nc(-c6ccccc6)n5)c5sc6ccccc6c45)c23)cc1 ; c1ccc(-c2ccc3oc4cccc(-c5ccc(-c6nc(-c7ccccc7)nc(-c7ccccc7)n6)c6sc7ccccc7c56)c4c3c2)cc1 ; c1ccc(-c2ccc(-c3nc(-c4ccc(-c5ccccc5)cc4)nc(-c4ccc(-c5cccc6c5oc5c(-c7ccccc7)cccc56)c5c4sc4ccccc45)n3)cc2)cc1]

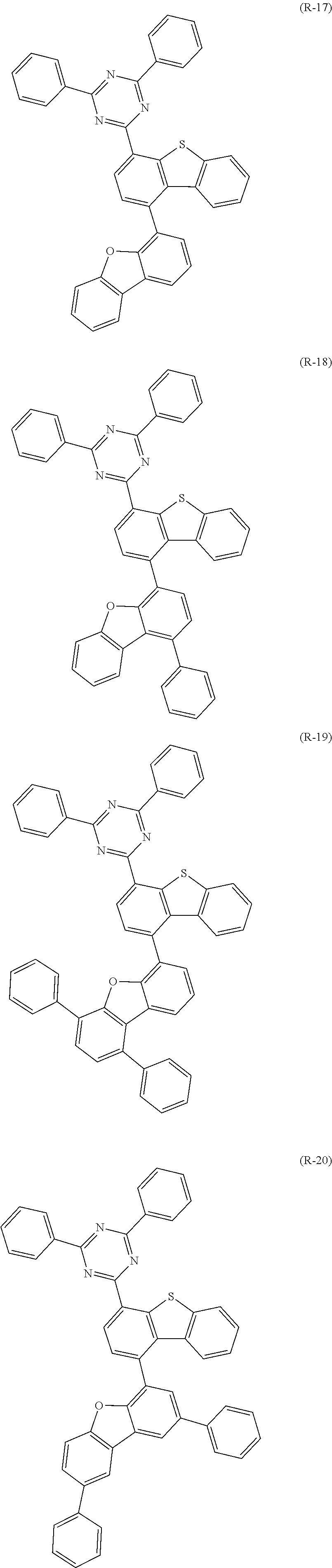

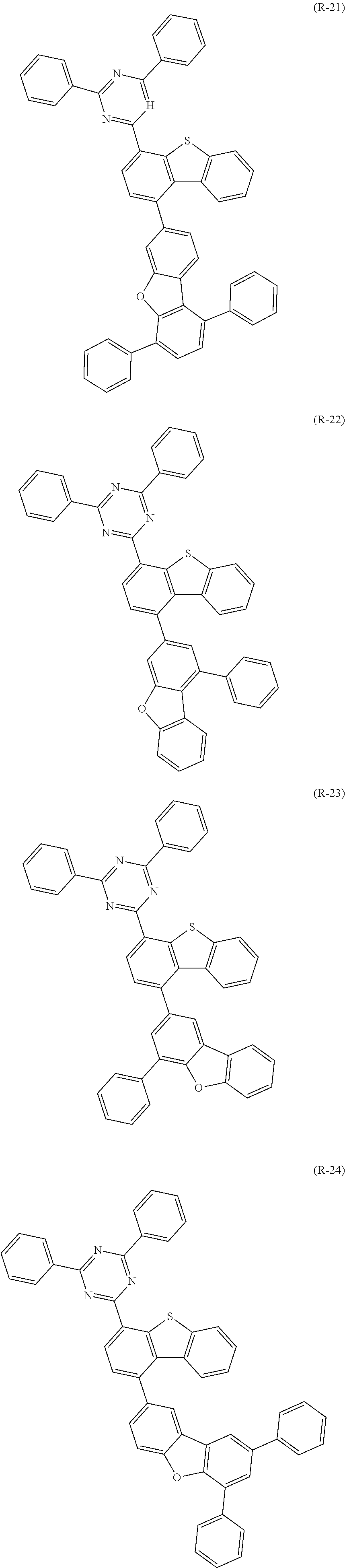

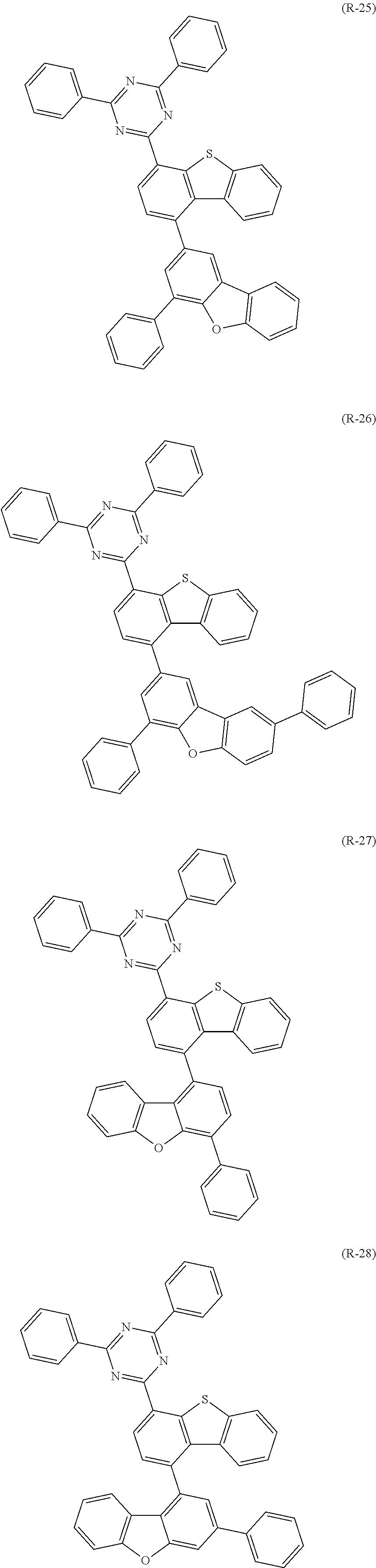 [SMILES: c1ccc(-c2nc(-c3ccccc3)nc(-c3ccc(-c4cc(-c5ccccc5)c5oc6ccccc6c5c4)c4c3sc3ccccc34)n2)cc1 ; c1ccc(-c2ccc3oc4c(-c5ccccc5)cc(-c5ccc(-c6nc(-c7ccccc7)nc(-c7ccccc7)n6)c6sc7ccccc7c56)cc4c3c2)cc1 ; c1ccc(-c2nc(-c3ccccc3)nc(-c3ccc(-c4ccc(-c5ccccc5)c5oc6ccccc6c45)c4c3sc3ccccc34)n2)cc1 ; c1ccc(-c2cc(-c3ccc(-c4nc(-c5ccccc5)nc(-c5ccccc5)n4)c4sc5ccccc5c34)c3c(c2)oc2ccccc23)cc1]

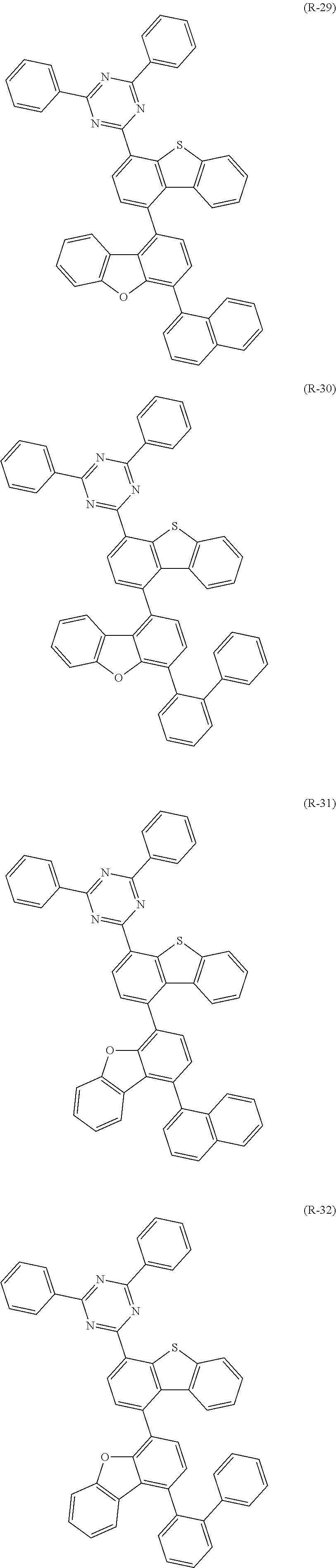 [SMILES: c1ccc(-c2nc(-c3ccccc3)nc(-c3ccc(-c4ccc(-c5cccc6ccccc56)c5oc6ccccc6c45)c4c3sc3ccccc34)n2)cc1 ; c1ccc(-c2nc(-c3ccccc3)nc(-c3ccc(-c4ccc(-c5ccccc5-c5ccccc5)c5oc6ccccc6c45)c4c3sc3ccccc34)n2)cc1 ; c1ccc(-c2nc(-c3ccccc3)nc(-c3ccc(-c4ccc(-c5cccc6ccccc56)c5c4oc4ccccc45)c4c3sc3ccccc34)n2)cc1 ; c1ccc(-c2nc(-c3ccccc3)nc(-c3ccc(-c4ccc(-c5ccccc5-c5ccccc5)c5c4oc4ccccc45)c4c3sc3ccccc34)n2)cc1]

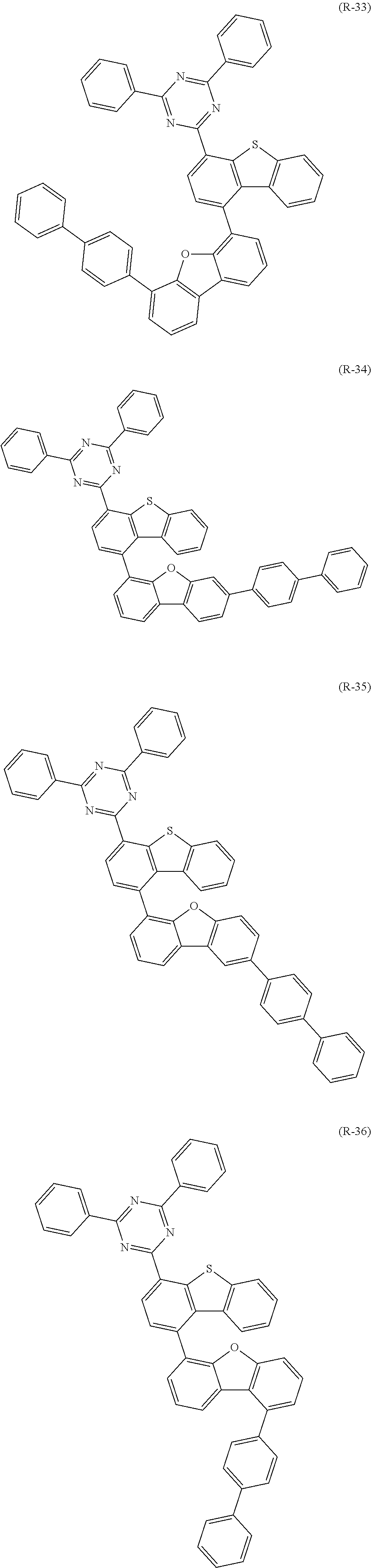 [SMILES: c1ccc(-c2ccc(-c3cccc4c3oc3c(-c5ccc(-c6nc(-c7ccccc7)nc(-c7ccccc7)n6)c6sc7ccccc7c56)cccc34)cc2)cc1 ; c1ccc(-c2ccc(-c3ccc4c(c3)oc3c(-c5ccc(-c6nc(-c7ccccc7)nc(-c7ccccc7)n6)c6sc7ccccc7c56)cccc34)cc2)cc1 ; c1ccc(-c2ccc(-c3ccc4oc5c(-c6ccc(-c7nc(-c8ccccc8)nc(-c8ccccc8)n7)c7sc8ccccc8c67)cccc5c4c3)cc2)cc1 ; c1ccc(-c2ccc(-c3cccc4oc5c(-c6ccc(-c7nc(-c8ccccc8)nc(-c8ccccc8)n7)c7sc8ccccc8c67)cccc5c34)cc2)cc1]

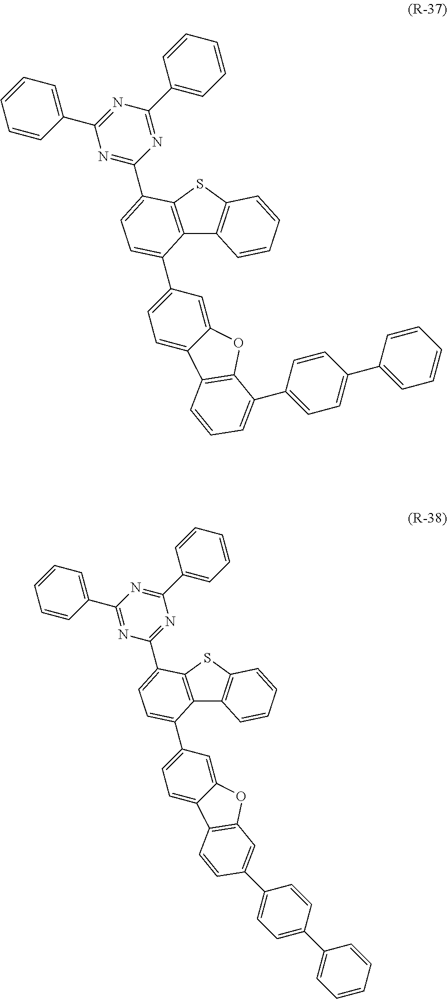 [SMILES: c1ccc(-c2ccc(-c3cccc4c3oc3cc(-c5ccc(-c6nc(-c7ccccc7)nc(-c7ccccc7)n6)c6sc7ccccc7c56)ccc34)cc2)cc1 ; c1ccc(-c2ccc(-c3ccc4c(c3)oc3cc(-c5ccc(-c6nc(-c7ccccc7)nc(-c7ccccc7)n6)c6sc7ccccc7c56)ccc34)cc2)cc1]

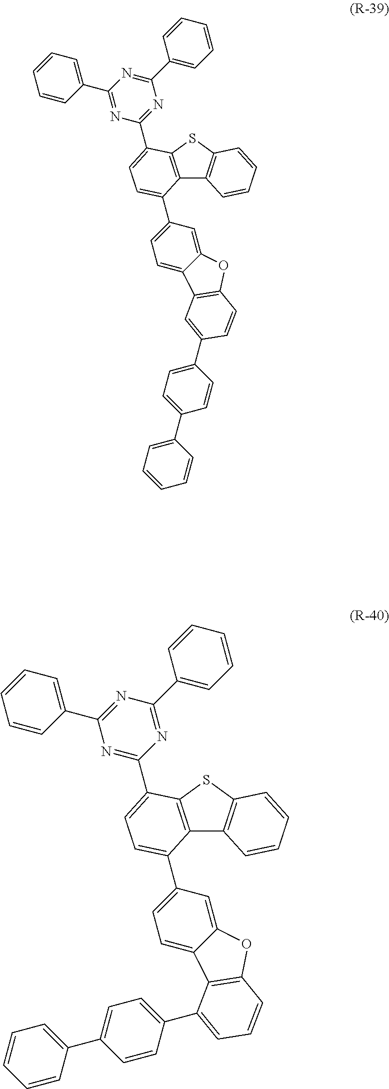 [SMILES: c1ccc(-c2ccc(-c3ccc4oc5cc(-c6ccc(-c7nc(-c8ccccc8)nc(-c8ccccc8)n7)c7sc8ccccc8c67)ccc5c4c3)cc2)cc1 ; c1ccc(-c2ccc(-c3cccc4oc5cc(-c6ccc(-c7nc(-c8ccccc8)nc(-c8ccccc8)n7)c7sc8ccccc8c67)ccc5c34)cc2)cc1]

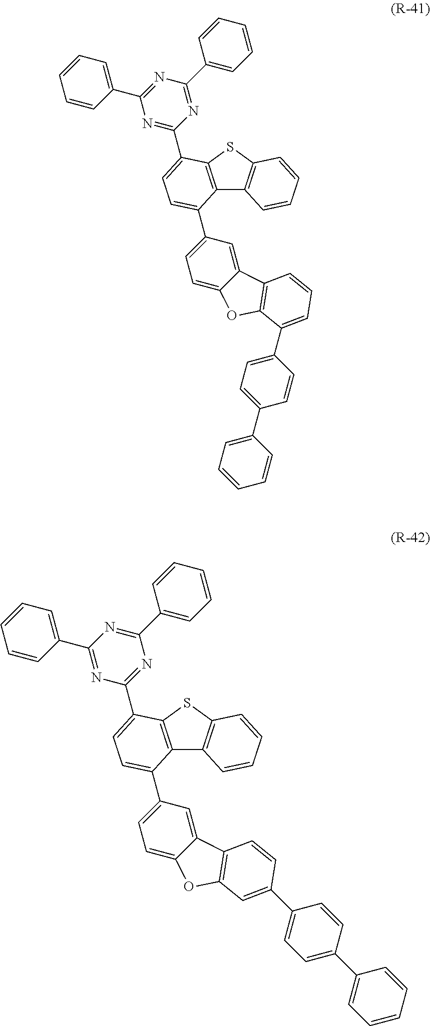 [SMILES: c1ccc(-c2ccc(-c3cccc4c3oc3ccc(-c5ccc(-c6nc(-c7ccccc7)nc(-c7ccccc7)n6)c6sc7ccccc7c56)cc34)cc2)cc1 ; c1ccc(-c2ccc(-c3ccc4c(c3)oc3ccc(-c5ccc(-c6nc(-c7ccccc7)nc(-c7ccccc7)n6)c6sc7ccccc7c56)cc34)cc2)cc1]

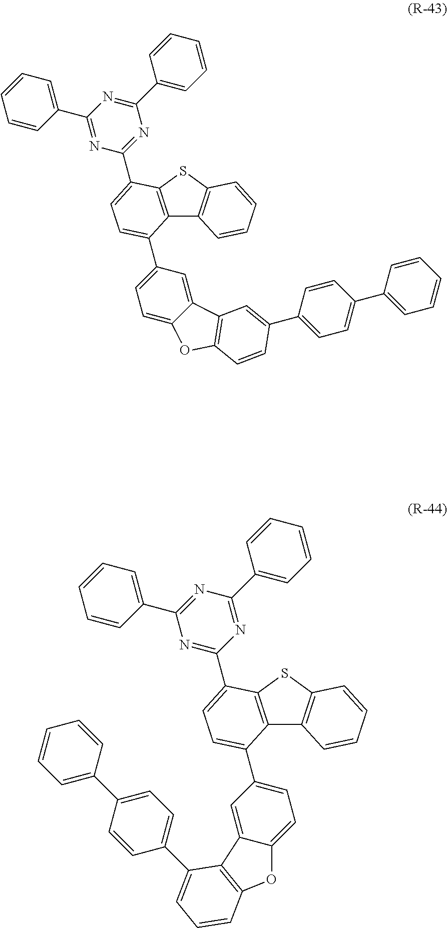 [SMILES: c1ccc(-c2ccc(-c3ccc4oc5ccc(-c6ccc(-c7nc(-c8ccccc8)nc(-c8ccccc8)n7)c7sc8ccccc8c67)cc5c4c3)cc2)cc1 ; c1ccc(-c2ccc(-c3cccc4oc5ccc(-c6ccc(-c7nc(-c8ccccc8)nc(-c8ccccc8)n7)c7sc8ccccc8c67)cc5c34)cc2)cc1]

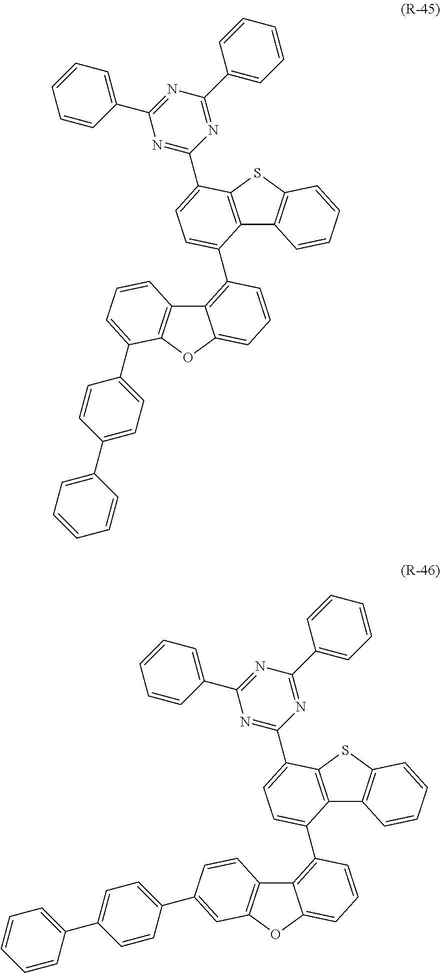 [SMILES: c1ccc(-c2ccc(-c3cccc4c3oc3cccc(-c5ccc(-c6nc(-c7ccccc7)nc(-c7ccccc7)n6)c6sc7ccccc7c56)c34)cc2)cc1 ; c1ccc(-c2ccc(-c3ccc4c(c3)oc3cccc(-c5ccc(-c6nc(-c7ccccc7)nc(-c7ccccc7)n6)c6sc7ccccc7c56)c34)cc2)cc1]

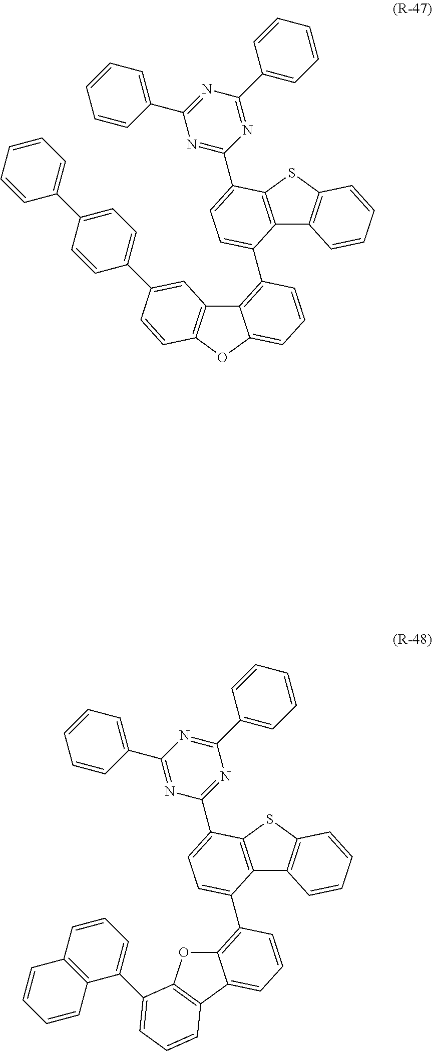 [SMILES: c1ccc(-c2ccc(-c3ccc4oc5cccc(-c6ccc(-c7nc(-c8ccccc8)nc(-c8ccccc8)n7)c7sc8ccccc8c67)c5c4c3)cc2)cc1 ; c1ccc(-c2nc(-c3ccccc3)nc(-c3ccc(-c4cccc5c4oc4c(-c6cccc7ccccc67)cccc45)c4c3sc3ccccc34)n2)cc1]

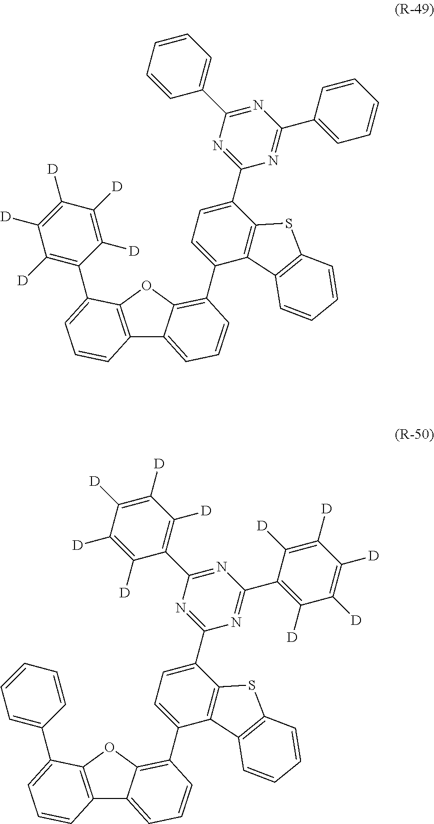 [SMILES: [2H]c1c([2H])c([2H])c(-c2cccc3c2oc2c(-c4ccc(-c5nc(-c6ccccc6)nc(-c6ccccc6)n5)c5sc6ccccc6c45)cccc23)c([2H])c1[2H] ; [2H]c1c([2H])c([2H])c(-c2nc(-c3c([2H])c([2H])c([2H])c([2H])c3[2H])nc(-c3ccc(-c4cccc5c4oc4c(-c6ccccc6)cccc45)c4c3sc3ccccc34)n2)c([2H])c1[2H]]

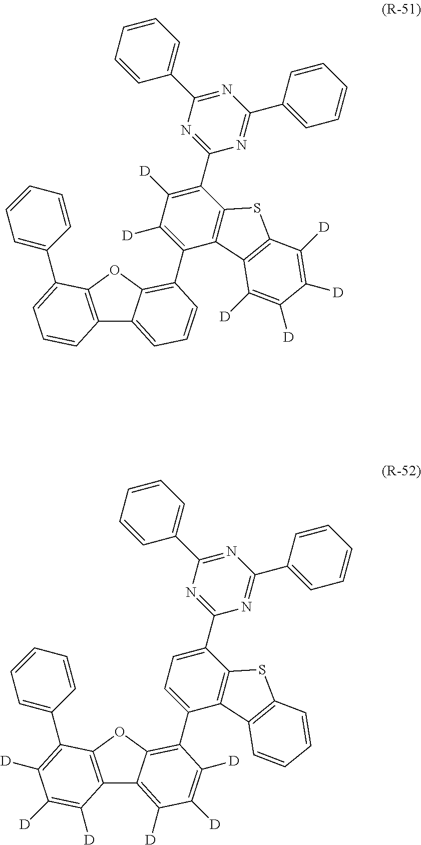 [SMILES: [2H]c1c([2H])c([2H])c2c(sc3c(-c4nc(-c5ccccc5)nc(-c5ccccc5)n4)c([2H])c([2H])c(-c4cccc5c4oc4c(-c6ccccc6)cccc45)c32)c1[2H] ; [2H]c1c([2H])c([2H])c2c(oc3c(-c4ccc(-c5nc(-c6ccccc6)nc(-c6ccccc6)n5)c5sc6ccccc6c45)c([2H])c([2H])c([2H])c32)c1-c1ccccc1]

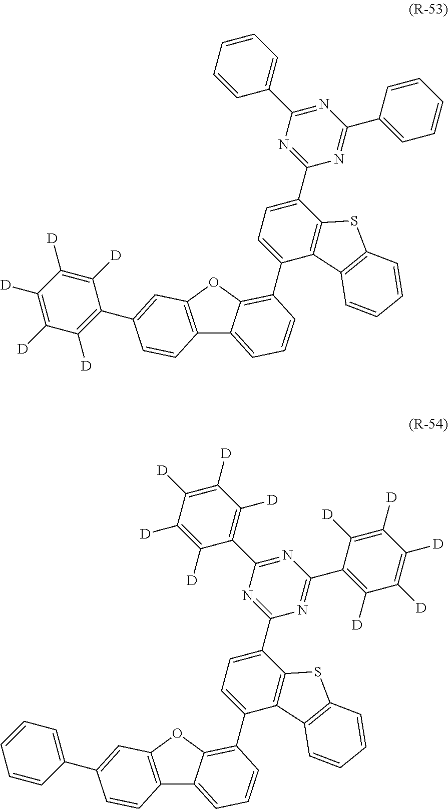 [SMILES: [2H]c1c([2H])c([2H])c(-c2ccc3c(c2)oc2c(-c4ccc(-c5nc(-c6ccccc6)nc(-c6ccccc6)n5)c5sc6ccccc6c45)cccc23)c([2H])c1[2H] ; [2H]c1c([2H])c([2H])c(-c2nc(-c3c([2H])c([2H])c([2H])c([2H])c3[2H])nc(-c3ccc(-c4cccc5c4oc4cc(-c6ccccc6)ccc45)c4c3sc3ccccc34)n2)c([2H])c1[2H]]

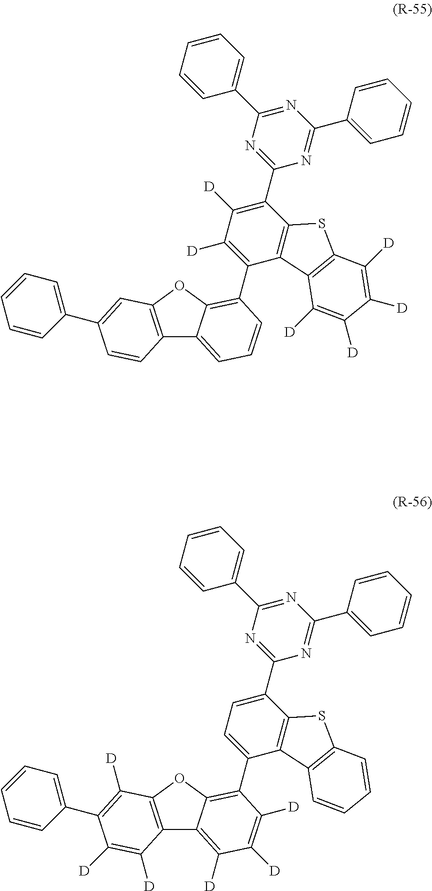 [SMILES: [2H]c1c([2H])c([2H])c2c(sc3c(-c4nc(-c5ccccc5)nc(-c5ccccc5)n4)c([2H])c([2H])c(-c4cccc5c4oc4cc(-c6ccccc6)ccc45)c32)c1[2H] ; [2H]c1c([2H])c([2H])c2c(oc3c([2H])c(-c4ccccc4)c([2H])c([2H])c32)c1-c1ccc(-c2nc(-c3ccccc3)nc(-c3ccccc3)n2)c2sc3ccccc3c12]

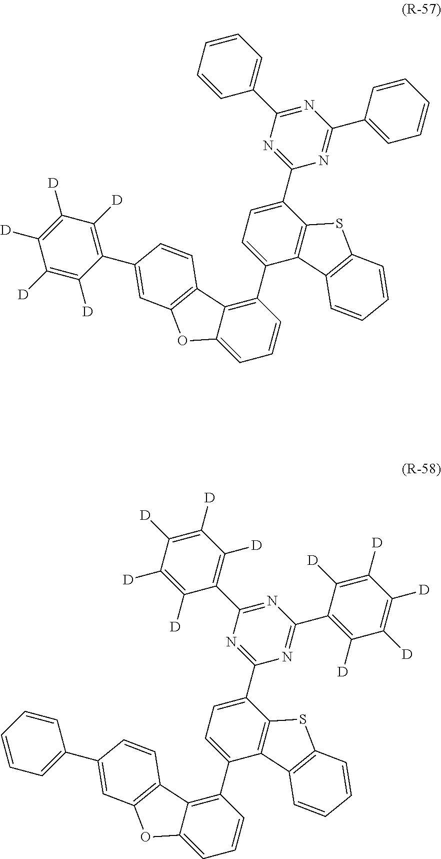 [SMILES: [2H]c1c([2H])c([2H])c(-c2ccc3c(c2)oc2cccc(-c4ccc(-c5nc(-c6ccccc6)nc(-c6ccccc6)n5)c5sc6ccccc6c45)c23)c([2H])c1[2H] ; [2H]c1c([2H])c([2H])c(-c2nc(-c3c([2H])c([2H])c([2H])c([2H])c3[2H])nc(-c3ccc(-c4cccc5oc6cc(-c7ccccc7)ccc6c45)c4c3sc3ccccc34)n2)c([2H])c1[2H]]

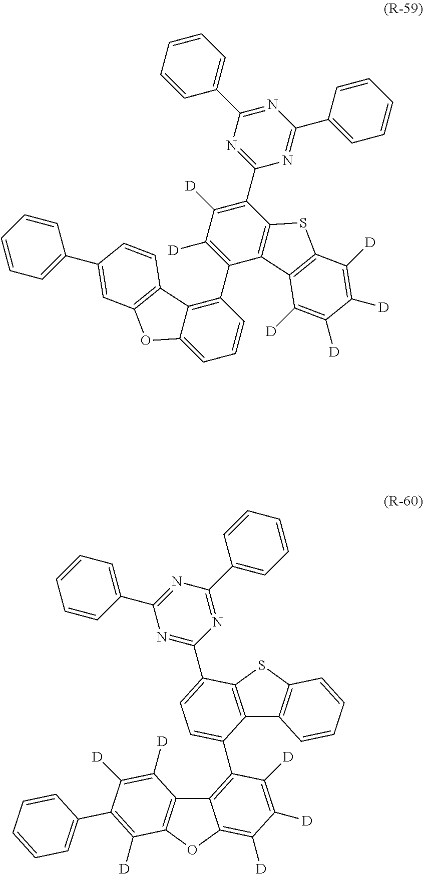 [SMILES: [2H]c1c([2H])c([2H])c2c(sc3c(-c4nc(-c5ccccc5)nc(-c5ccccc5)n4)c([2H])c([2H])c(-c4cccc5oc6cc(-c7ccccc7)ccc6c45)c32)c1[2H] ; [2H]c1c([2H])c(-c2ccc(-c3nc(-c4ccccc4)nc(-c4ccccc4)n3)c3sc4ccccc4c23)c2c(oc3c([2H])c(-c4ccccc4)c([2H])c([2H])c32)c1[2H]]

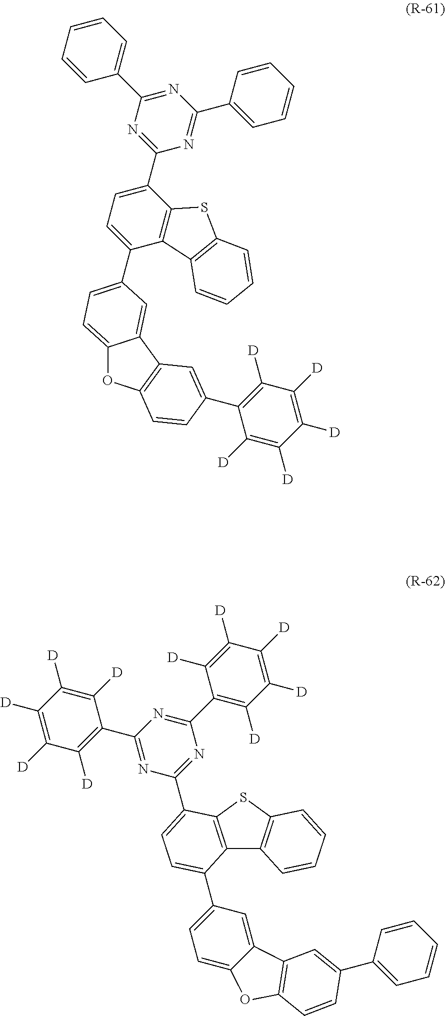 [SMILES: [2H]c1c([2H])c([2H])c(-c2ccc3oc4ccc(-c5ccc(-c6nc(-c7ccccc7)nc(-c7ccccc7)n6)c6sc7ccccc7c56)cc4c3c2)c([2H])c1[2H] ; [2H]c1c([2H])c([2H])c(-c2nc(-c3c([2H])c([2H])c([2H])c([2H])c3[2H])nc(-c3ccc(-c4ccc5oc6ccc(-c7ccccc7)cc6c5c4)c4c3sc3ccccc34)n2)c([2H])c1[2H]]

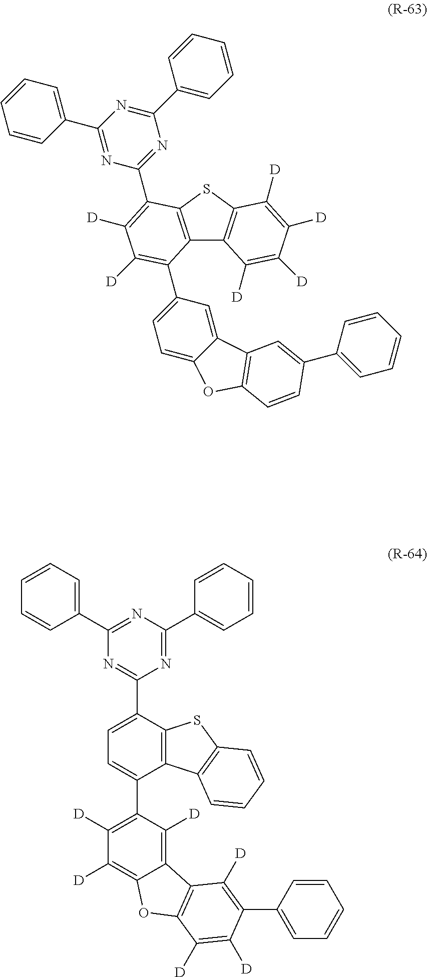 [SMILES: [2H]c1c([2H])c([2H])c2c(sc3c(-c4nc(-c5ccccc5)nc(-c5ccccc5)n4)c([2H])c([2H])c(-c4ccc5oc6ccc(-c7ccccc7)cc6c5c4)c32)c1[2H] ; [2H]c1c(-c2ccccc2)c([2H])c2c(oc3c([2H])c([2H])c(-c4ccc(-c5nc(-c6ccccc6)nc(-c6ccccc6)n5)c5sc6ccccc6c45)c([2H])c32)c1[2H]]

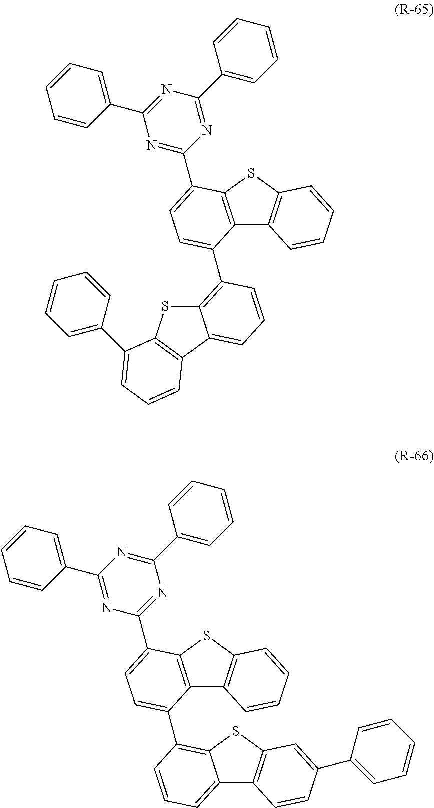 [SMILES: c1ccc(-c2nc(-c3ccccc3)nc(-c3ccc(-c4cccc5c4sc4c(-c6ccccc6)cccc45)c4c3sc3ccccc34)n2)cc1 ; c1ccc(-c2ccc3c(c2)sc2c(-c4ccc(-c5nc(-c6ccccc6)nc(-c6ccccc6)n5)c5sc6ccccc6c45)cccc23)cc1]

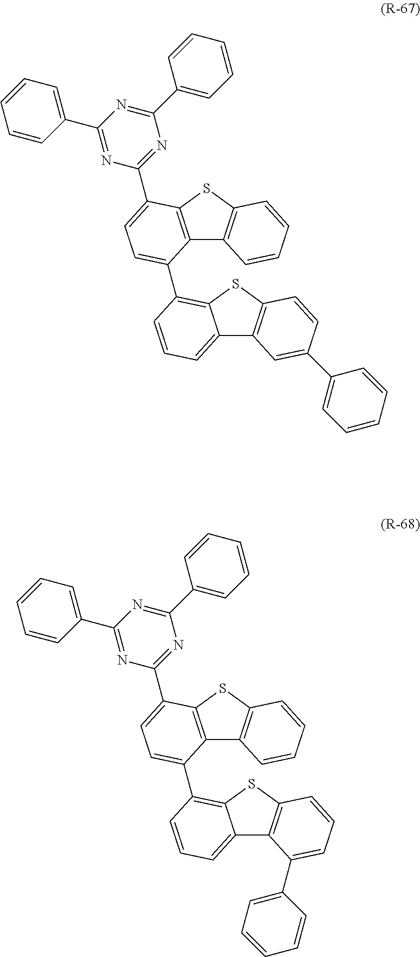 [SMILES: c1ccc(-c2ccc3sc4c(-c5ccc(-c6nc(-c7ccccc7)nc(-c7ccccc7)n6)c6sc7ccccc7c56)cccc4c3c2)cc1 ; c1ccc(-c2nc(-c3ccccc3)nc(-c3ccc(-c4cccc5c4sc4cccc(-c6ccccc6)c45)c4c3sc3ccccc34)n2)cc1]

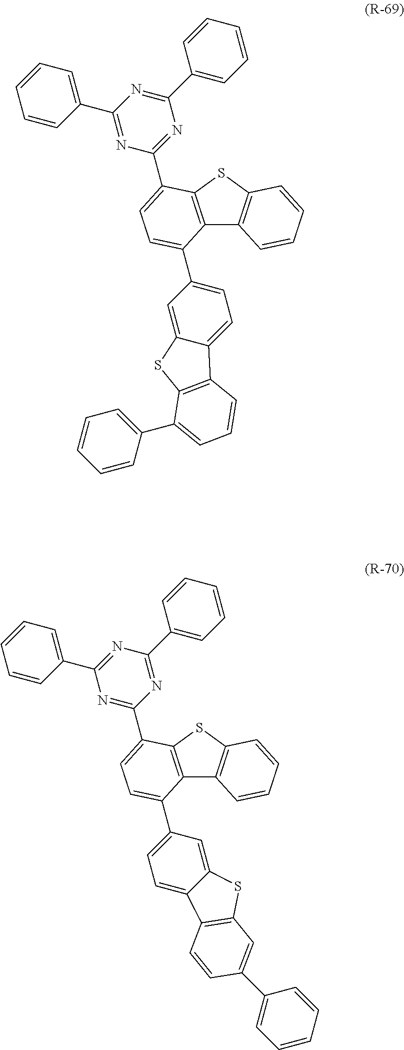 [SMILES: c1ccc(-c2nc(-c3ccccc3)nc(-c3ccc(-c4ccc5c(c4)sc4c(-c6ccccc6)cccc45)c4c3sc3ccccc34)n2)cc1 ; c1ccc(-c2ccc3c(c2)sc2cc(-c4ccc(-c5nc(-c6ccccc6)nc(-c6ccccc6)n5)c5sc6ccccc6c45)ccc23)cc1]

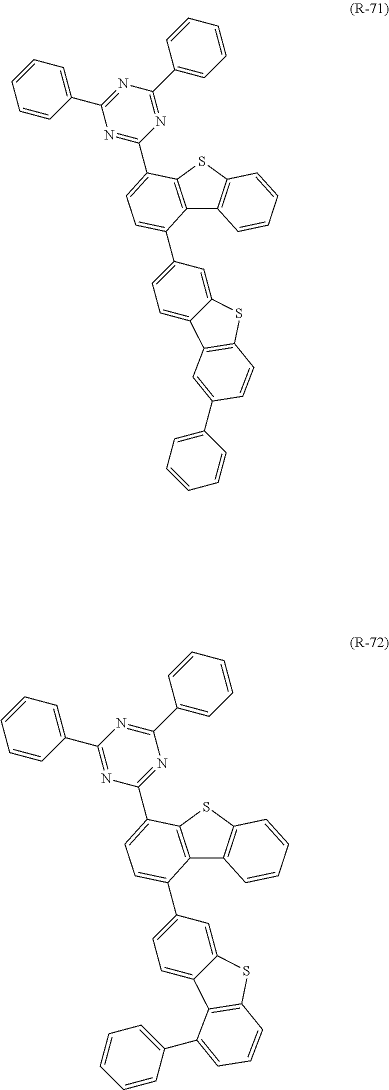 [SMILES: c1ccc(-c2ccc3sc4cc(-c5ccc(-c6nc(-c7ccccc7)nc(-c7ccccc7)n6)c6sc7ccccc7c56)ccc4c3c2)cc1 ; c1ccc(-c2nc(-c3ccccc3)nc(-c3ccc(-c4ccc5c(c4)sc4cccc(-c6ccccc6)c45)c4c3sc3ccccc34)n2)cc1]

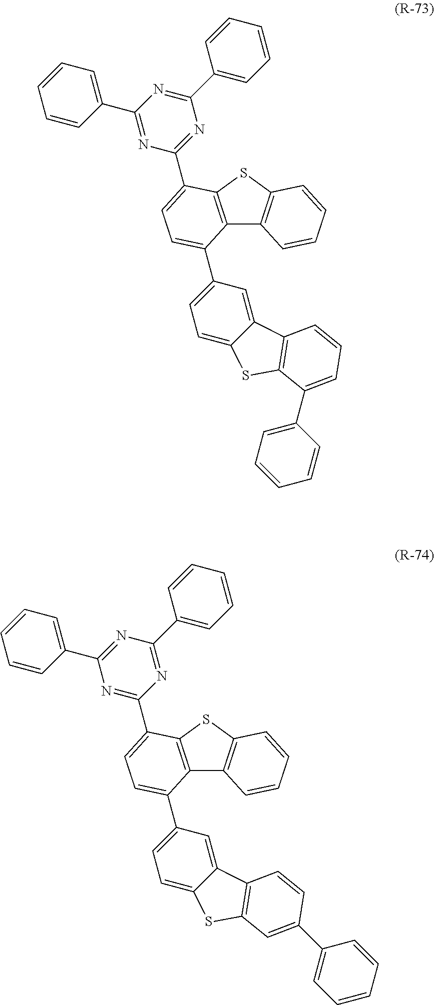 [SMILES: c1ccc(-c2nc(-c3ccccc3)nc(-c3ccc(-c4ccc5sc6c(-c7ccccc7)cccc6c5c4)c4c3sc3ccccc34)n2)cc1 ; c1ccc(-c2ccc3c(c2)sc2ccc(-c4ccc(-c5nc(-c6ccccc6)nc(-c6ccccc6)n5)c5sc6ccccc6c45)cc23)cc1]

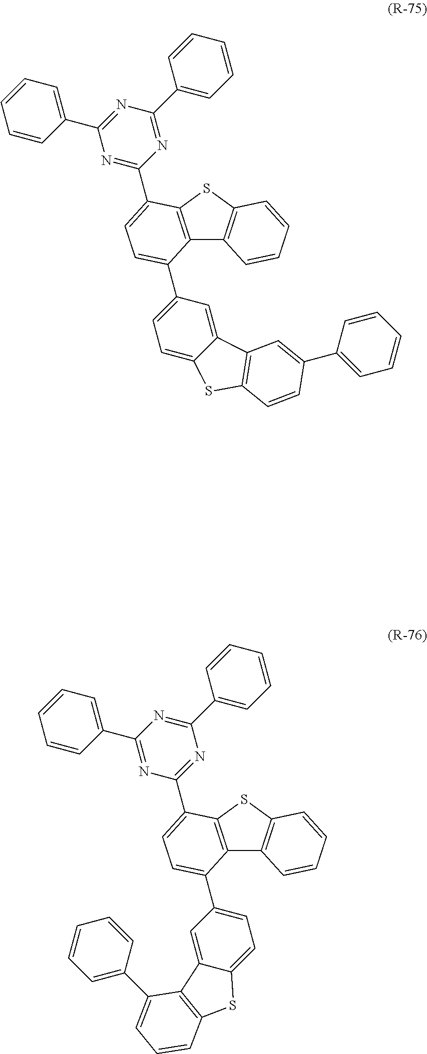 [SMILES: c1ccc(-c2ccc3sc4ccc(-c5ccc(-c6nc(-c7ccccc7)nc(-c7ccccc7)n6)c6sc7ccccc7c56)cc4c3c2)cc1 ; c1ccc(-c2nc(-c3ccccc3)nc(-c3ccc(-c4ccc5sc6cccc(-c7ccccc7)c6c5c4)c4c3sc3ccccc34)n2)cc1]

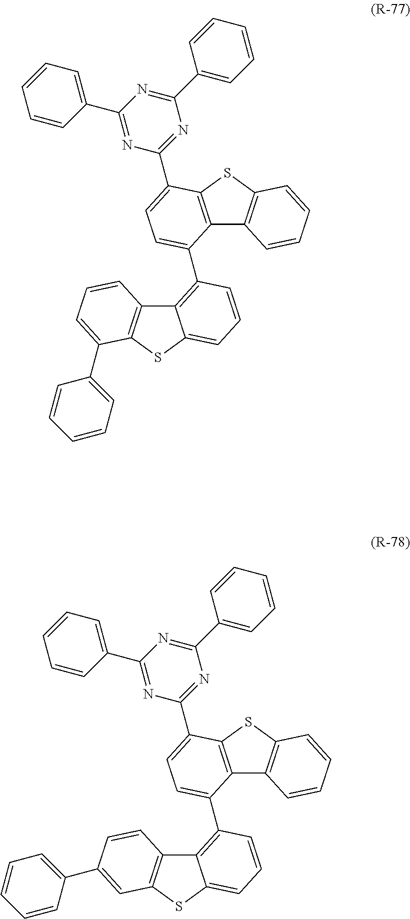 [SMILES: c1ccc(-c2nc(-c3ccccc3)nc(-c3ccc(-c4cccc5sc6c(-c7ccccc7)cccc6c45)c4c3sc3ccccc34)n2)cc1 ; c1ccc(-c2ccc3c(c2)sc2cccc(-c4ccc(-c5nc(-c6ccccc6)nc(-c6ccccc6)n5)c5sc6ccccc6c45)c23)cc1]

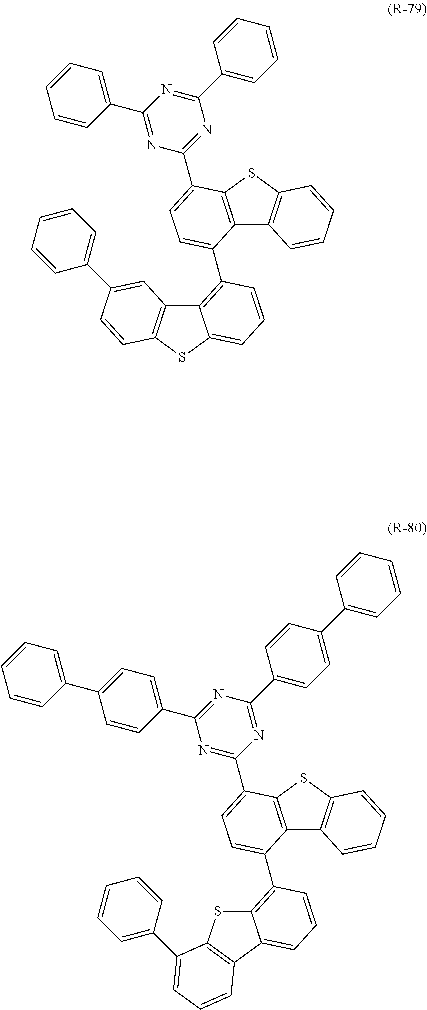 [SMILES: c1ccc(-c2ccc3sc4cccc(-c5ccc(-c6nc(-c7ccccc7)nc(-c7ccccc7)n6)c6sc7ccccc7c56)c4c3c2)cc1 ; c1ccc(-c2ccc(-c3nc(-c4ccc(-c5ccccc5)cc4)nc(-c4ccc(-c5cccc6c5sc5c(-c7ccccc7)cccc56)c5c4sc4ccccc45)n3)cc2)cc1]

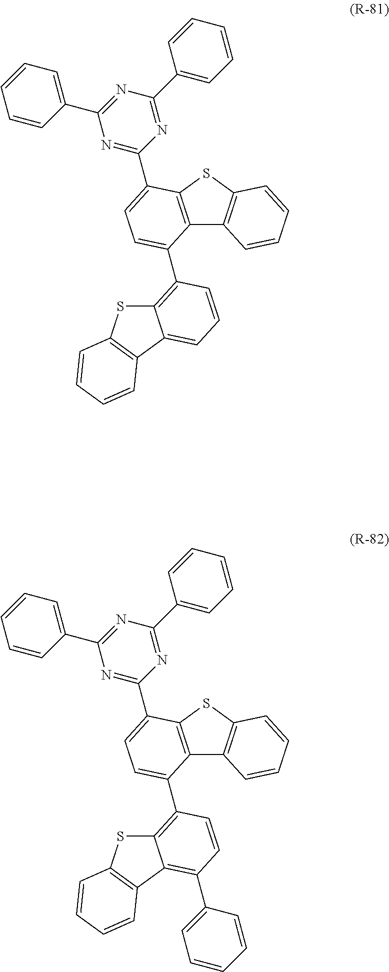 [SMILES: c1ccc(-c2nc(-c3ccccc3)nc(-c3ccc(-c4cccc5c4sc4ccccc45)c4c3sc3ccccc34)n2)cc1 ; c1ccc(-c2nc(-c3ccccc3)nc(-c3ccc(-c4ccc(-c5ccccc5)c5c4sc4ccccc45)c4c3sc3ccccc34)n2)cc1]

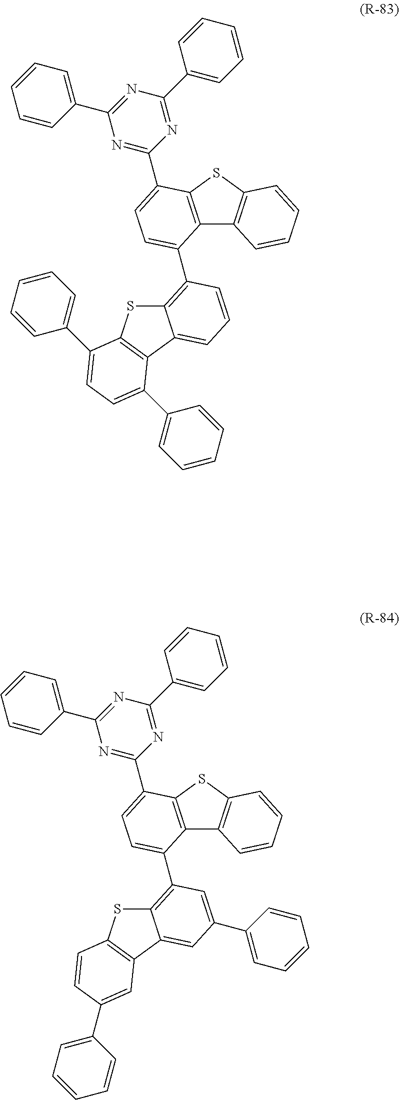 [SMILES: c1ccc(-c2nc(-c3ccccc3)nc(-c3ccc(-c4cccc5c4sc4c(-c6ccccc6)ccc(-c6ccccc6)c45)c4c3sc3ccccc34)n2)cc1 ; c1ccc(-c2ccc3sc4c(-c5ccc(-c6nc(-c7ccccc7)nc(-c7ccccc7)n6)c6sc7ccccc7c56)cc(-c5ccccc5)cc4c3c2)cc1]

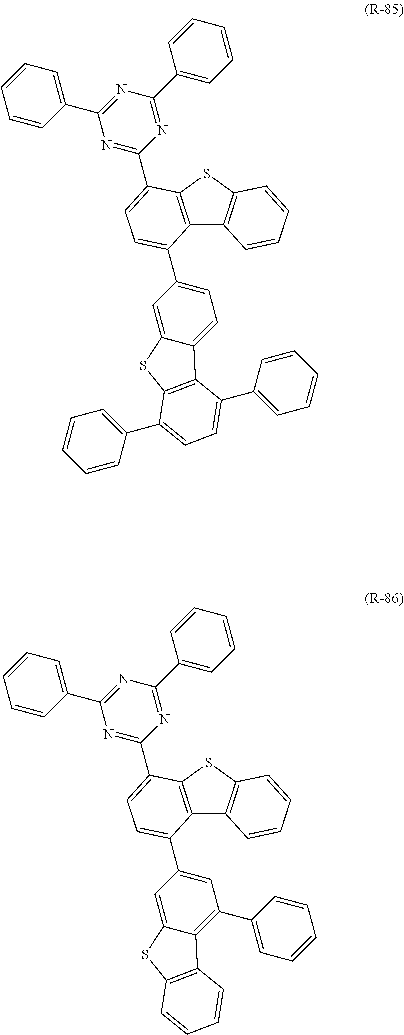 [SMILES: c1ccc(-c2nc(-c3ccccc3)nc(-c3ccc(-c4ccc5c(c4)sc4c(-c6ccccc6)ccc(-c6ccccc6)c45)c4c3sc3ccccc34)n2)cc1 ; c1ccc(-c2nc(-c3ccccc3)nc(-c3ccc(-c4cc(-c5ccccc5)c5c(c4)sc4ccccc45)c4c3sc3ccccc34)n2)cc1]

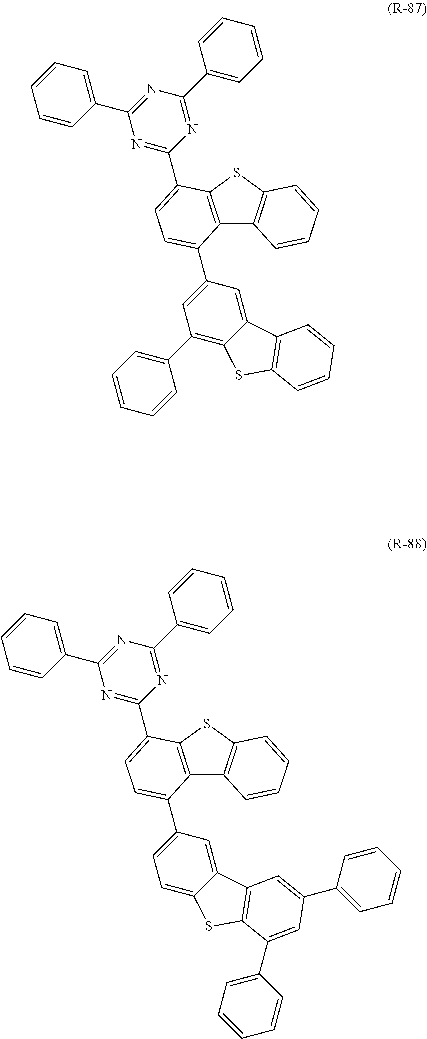 [SMILES: c1ccc(-c2nc(-c3ccccc3)nc(-c3ccc(-c4cc(-c5ccccc5)c5sc6ccccc6c5c4)c4c3sc3ccccc34)n2)cc1 ; c1ccc(-c2cc(-c3ccccc3)c3sc4ccc(-c5ccc(-c6nc(-c7ccccc7)nc(-c7ccccc7)n6)c6sc7ccccc7c56)cc4c3c2)cc1]

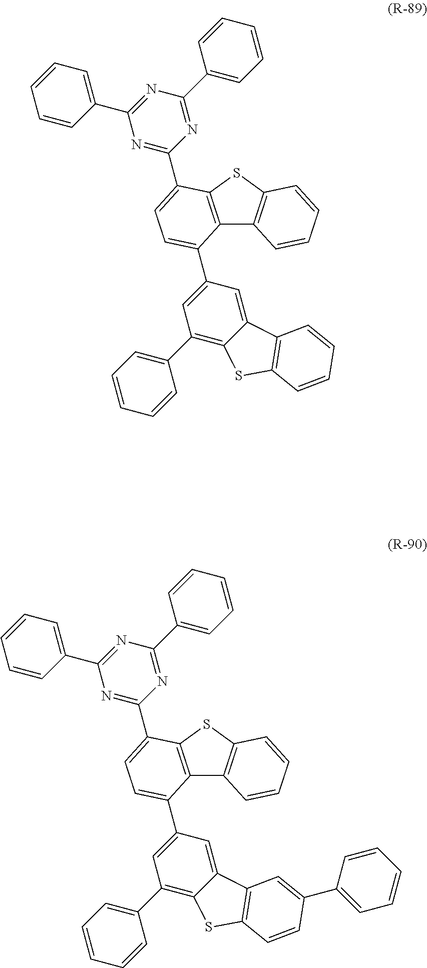 [SMILES: c1ccc(-c2nc(-c3ccccc3)nc(-c3ccc(-c4cc(-c5ccccc5)c5sc6ccccc6c5c4)c4c3sc3ccccc34)n2)cc1 ; c1ccc(-c2ccc3sc4c(-c5ccccc5)cc(-c5ccc(-c6nc(-c7ccccc7)nc(-c7ccccc7)n6)c6sc7ccccc7c56)cc4c3c2)cc1]

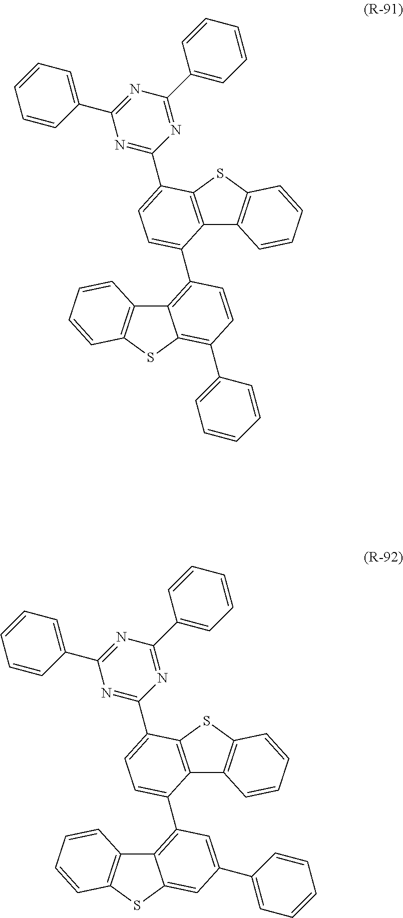 [SMILES: c1ccc(-c2nc(-c3ccccc3)nc(-c3ccc(-c4ccc(-c5ccccc5)c5sc6ccccc6c45)c4c3sc3ccccc34)n2)cc1 ; c1ccc(-c2cc(-c3ccc(-c4nc(-c5ccccc5)nc(-c5ccccc5)n4)c4sc5ccccc5c34)c3c(c2)sc2ccccc23)cc1]

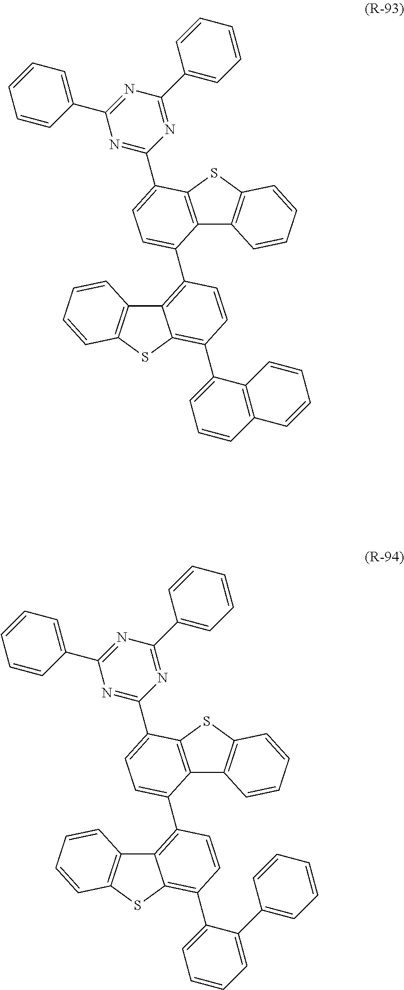 [SMILES: c1ccc(-c2nc(-c3ccccc3)nc(-c3ccc(-c4ccc(-c5cccc6ccccc56)c5sc6ccccc6c45)c4c3sc3ccccc34)n2)cc1 ; c1ccc(-c2nc(-c3ccccc3)nc(-c3ccc(-c4ccc(-c5ccccc5-c5ccccc5)c5sc6ccccc6c45)c4c3sc3ccccc34)n2)cc1]

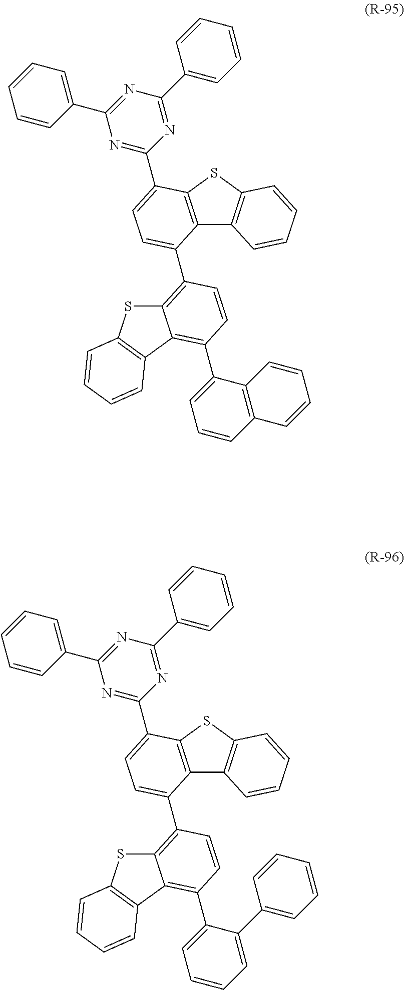 [SMILES: c1ccc(-c2nc(-c3ccccc3)nc(-c3ccc(-c4ccc(-c5cccc6ccccc56)c5c4sc4ccccc45)c4c3sc3ccccc34)n2)cc1 ; c1ccc(-c2nc(-c3ccccc3)nc(-c3ccc(-c4ccc(-c5ccccc5-c5ccccc5)c5c4sc4ccccc45)c4c3sc3ccccc34)n2)cc1]

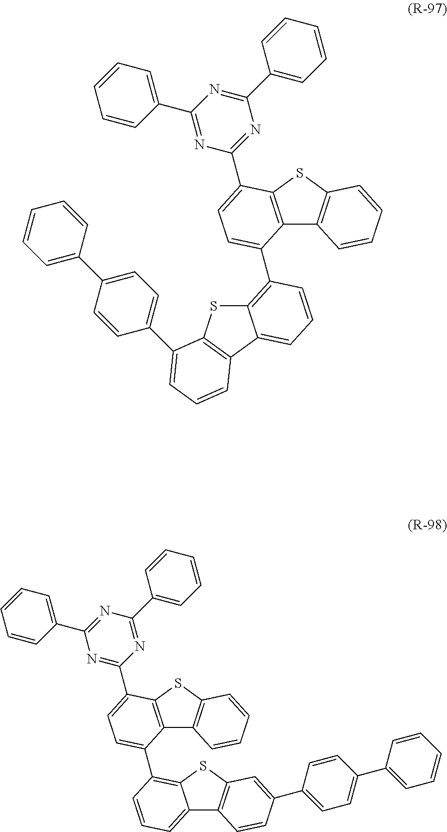 [SMILES: c1ccc(-c2ccc(-c3cccc4c3sc3c(-c5ccc(-c6nc(-c7ccccc7)nc(-c7ccccc7)n6)c6sc7ccccc7c56)cccc34)cc2)cc1 ; c1ccc(-c2ccc(-c3ccc4c(c3)sc3c(-c5ccc(-c6nc(-c7ccccc7)nc(-c7ccccc7)n6)c6sc7ccccc7c56)cccc34)cc2)cc1]

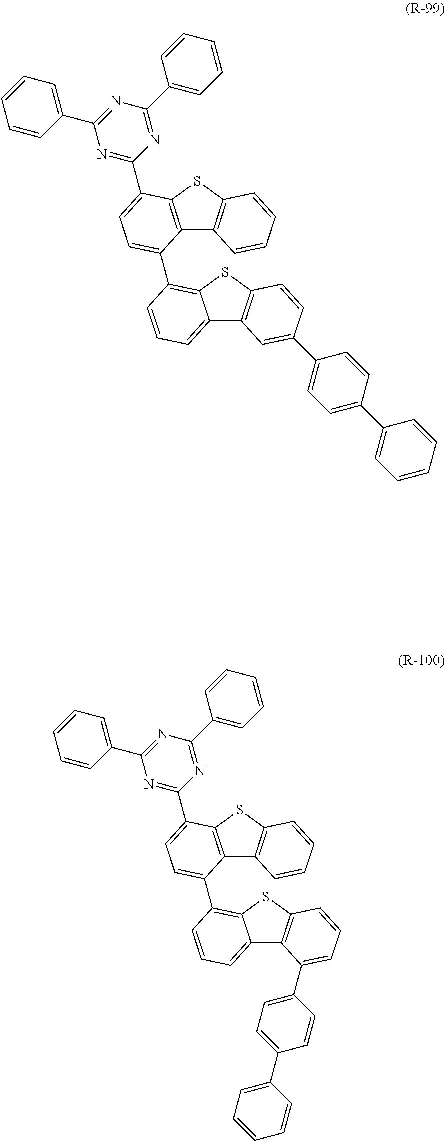 [SMILES: c1ccc(-c2ccc(-c3ccc4sc5c(-c6ccc(-c7nc(-c8ccccc8)nc(-c8ccccc8)n7)c7sc8ccccc8c67)cccc5c4c3)cc2)cc1 ; c1ccc(-c2ccc(-c3cccc4sc5c(-c6ccc(-c7nc(-c8ccccc8)nc(-c8ccccc8)n7)c7sc8ccccc8c67)cccc5c34)cc2)cc1]

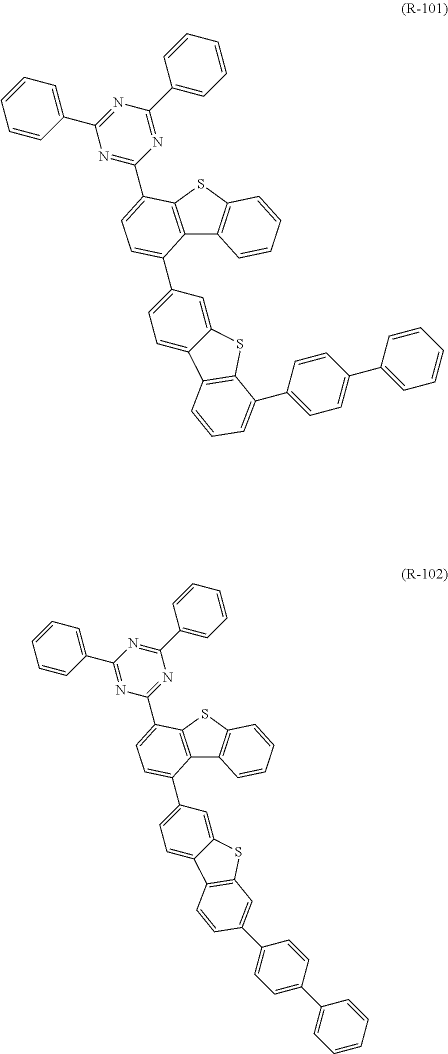 [SMILES: c1ccc(-c2ccc(-c3cccc4c3sc3cc(-c5ccc(-c6nc(-c7ccccc7)nc(-c7ccccc7)n6)c6sc7ccccc7c56)ccc34)cc2)cc1 ; c1ccc(-c2ccc(-c3ccc4c(c3)sc3cc(-c5ccc(-c6nc(-c7ccccc7)nc(-c7ccccc7)n6)c6sc7ccccc7c56)ccc34)cc2)cc1]

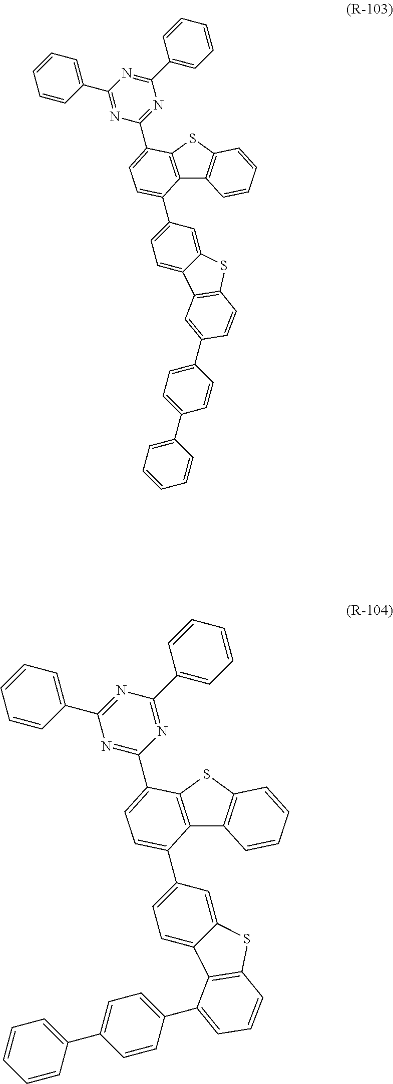 [SMILES: c1ccc(-c2ccc(-c3ccc4sc5cc(-c6ccc(-c7nc(-c8ccccc8)nc(-c8ccccc8)n7)c7sc8ccccc8c67)ccc5c4c3)cc2)cc1 ; c1ccc(-c2ccc(-c3cccc4sc5cc(-c6ccc(-c7nc(-c8ccccc8)nc(-c8ccccc8)n7)c7sc8ccccc8c67)ccc5c34)cc2)cc1]

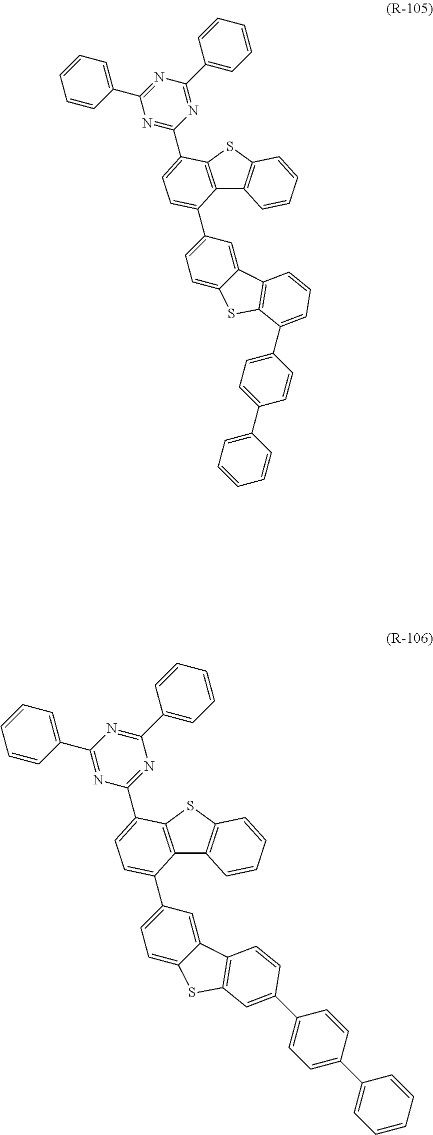 [SMILES: c1ccc(-c2ccc(-c3cccc4c3sc3ccc(-c5ccc(-c6nc(-c7ccccc7)nc(-c7ccccc7)n6)c6sc7ccccc7c56)cc34)cc2)cc1 ; c1ccc(-c2ccc(-c3ccc4c(c3)sc3ccc(-c5ccc(-c6nc(-c7ccccc7)nc(-c7ccccc7)n6)c6sc7ccccc7c56)cc34)cc2)cc1]

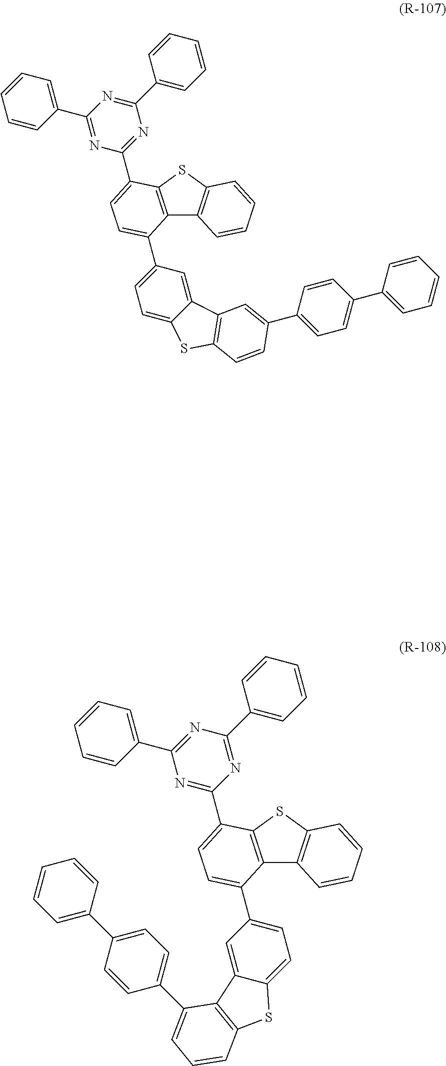 [SMILES: c1ccc(-c2ccc(-c3ccc4sc5ccc(-c6ccc(-c7nc(-c8ccccc8)nc(-c8ccccc8)n7)c7sc8ccccc8c67)cc5c4c3)cc2)cc1 ; c1ccc(-c2ccc(-c3cccc4sc5ccc(-c6ccc(-c7nc(-c8ccccc8)nc(-c8ccccc8)n7)c7sc8ccccc8c67)cc5c34)cc2)cc1]

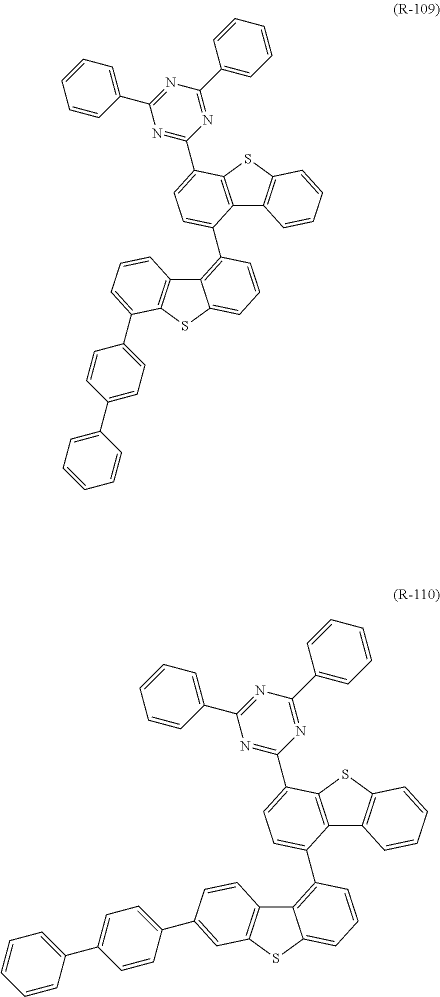 [SMILES: c1ccc(-c2ccc(-c3cccc4c3sc3cccc(-c5ccc(-c6nc(-c7ccccc7)nc(-c7ccccc7)n6)c6sc7ccccc7c56)c34)cc2)cc1 ; c1ccc(-c2ccc(-c3ccc4c(c3)sc3cccc(-c5ccc(-c6nc(-c7ccccc7)nc(-c7ccccc7)n6)c6sc7ccccc7c56)c34)cc2)cc1]

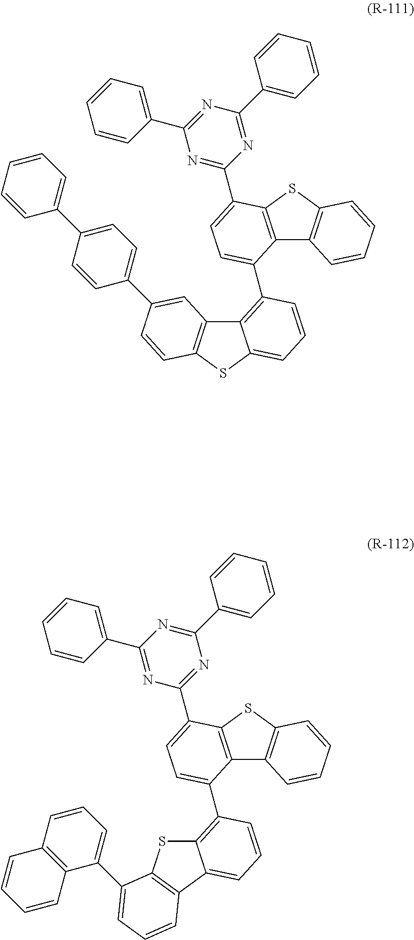 [SMILES: c1ccc(-c2ccc(-c3ccc4sc5cccc(-c6ccc(-c7nc(-c8ccccc8)nc(-c8ccccc8)n7)c7sc8ccccc8c67)c5c4c3)cc2)cc1 ; c1ccc(-c2nc(-c3ccccc3)nc(-c3ccc(-c4cccc5c4sc4c(-c6cccc7ccccc67)cccc45)c4c3sc3ccccc34)n2)cc1]

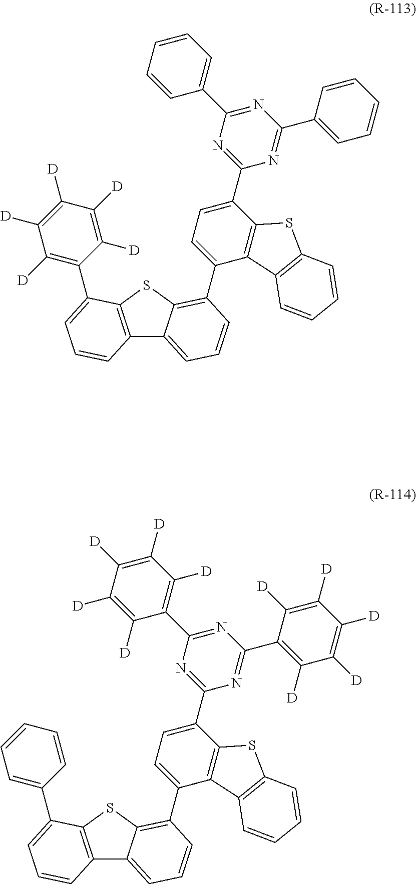 [SMILES: [2H]c1c([2H])c([2H])c(-c2cccc3c2sc2c(-c4ccc(-c5nc(-c6ccccc6)nc(-c6ccccc6)n5)c5sc6ccccc6c45)cccc23)c([2H])c1[2H] ; [2H]c1c([2H])c([2H])c(-c2nc(-c3c([2H])c([2H])c([2H])c([2H])c3[2H])nc(-c3ccc(-c4cccc5c4sc4c(-c6ccccc6)cccc45)c4c3sc3ccccc34)n2)c([2H])c1[2H]]

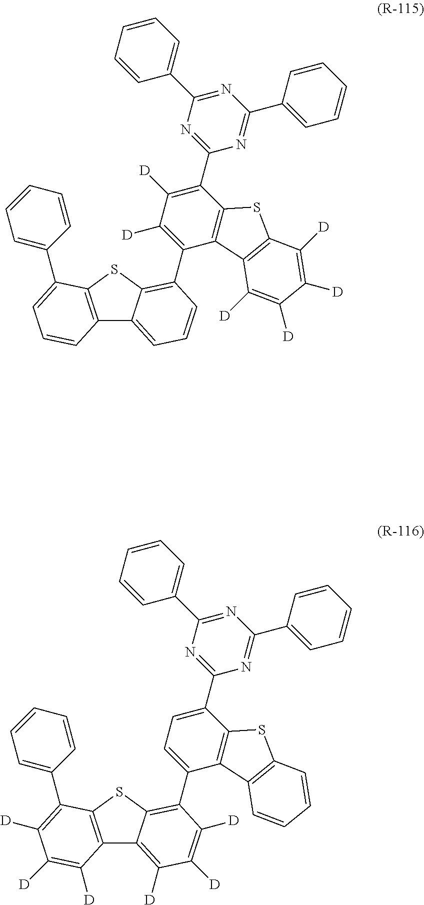 [SMILES: [2H]c1c([2H])c([2H])c2c(sc3c(-c4nc(-c5ccccc5)nc(-c5ccccc5)n4)c([2H])c([2H])c(-c4cccc5c4sc4c(-c6ccccc6)cccc45)c32)c1[2H] ; [2H]c1c([2H])c([2H])c2c(sc3c(-c4ccc(-c5nc(-c6ccccc6)nc(-c6ccccc6)n5)c5sc6ccccc6c45)c([2H])c([2H])c([2H])c32)c1-c1ccccc1]

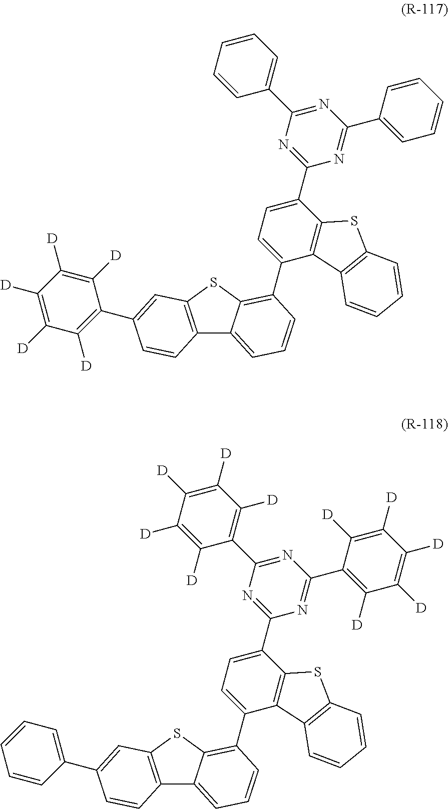 [SMILES: [2H]c1c([2H])c([2H])c(-c2ccc3c(c2)sc2c(-c4ccc(-c5nc(-c6ccccc6)nc(-c6ccccc6)n5)c5sc6ccccc6c45)cccc23)c([2H])c1[2H] ; [2H]c1c([2H])c([2H])c(-c2nc(-c3c([2H])c([2H])c([2H])c([2H])c3[2H])nc(-c3ccc(-c4cccc5c4sc4cc(-c6ccccc6)ccc45)c4c3sc3ccccc34)n2)c([2H])c1[2H]]

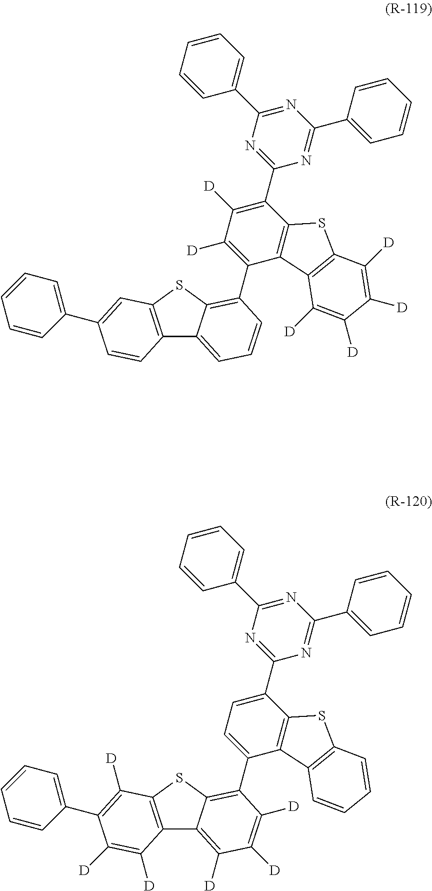 [SMILES: [2H]c1c([2H])c([2H])c2c(sc3c(-c4nc(-c5ccccc5)nc(-c5ccccc5)n4)c([2H])c([2H])c(-c4cccc5c4sc4cc(-c6ccccc6)ccc45)c32)c1[2H] ; [2H]c1c([2H])c([2H])c2c(sc3c([2H])c(-c4ccccc4)c([2H])c([2H])c32)c1-c1ccc(-c2nc(-c3ccccc3)nc(-c3ccccc3)n2)c2sc3ccccc3c12]

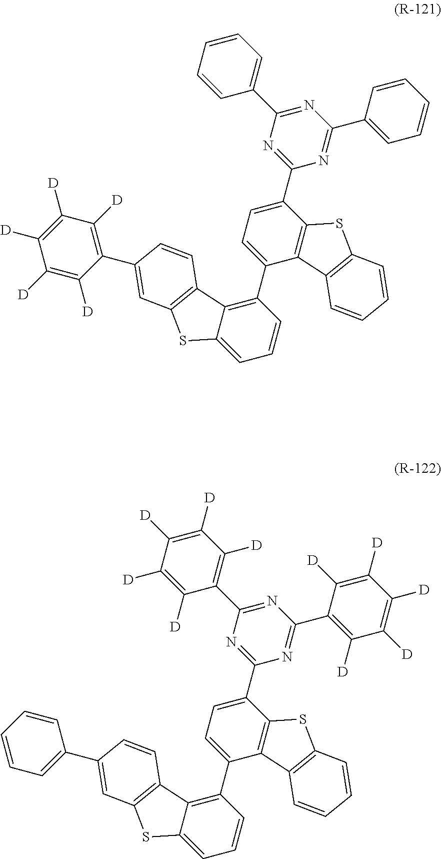 [SMILES: [2H]c1c([2H])c([2H])c(-c2ccc3c(c2)sc2cccc(-c4ccc(-c5nc(-c6ccccc6)nc(-c6ccccc6)n5)c5sc6ccccc6c45)c23)c([2H])c1[2H] ; [2H]c1c([2H])c([2H])c(-c2nc(-c3c([2H])c([2H])c([2H])c([2H])c3[2H])nc(-c3ccc(-c4cccc5sc6cc(-c7ccccc7)ccc6c45)c4c3sc3ccccc34)n2)c([2H])c1[2H]]

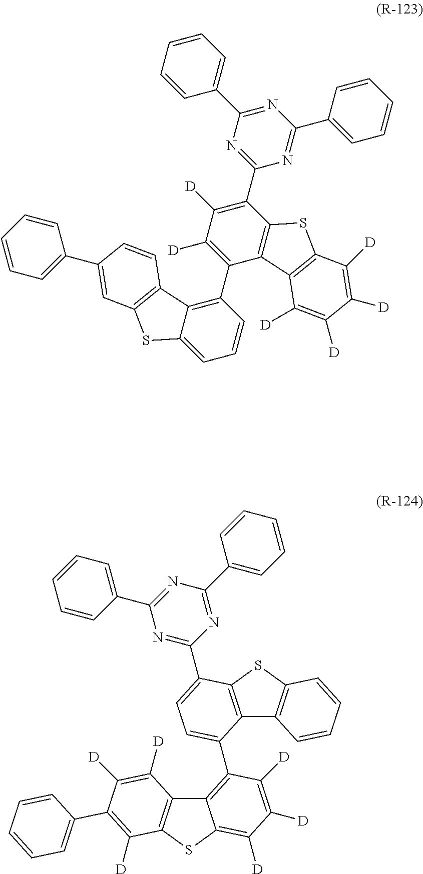 [SMILES: [2H]c1c([2H])c([2H])c2c(sc3c(-c4nc(-c5ccccc5)nc(-c5ccccc5)n4)c([2H])c([2H])c(-c4cccc5sc6cc(-c7ccccc7)ccc6c45)c32)c1[2H] ; [2H]c1c([2H])c(-c2ccc(-c3nc(-c4ccccc4)nc(-c4ccccc4)n3)c3sc4ccccc4c23)c2c(sc3c([2H])c(-c4ccccc4)c([2H])c([2H])c32)c1[2H]]

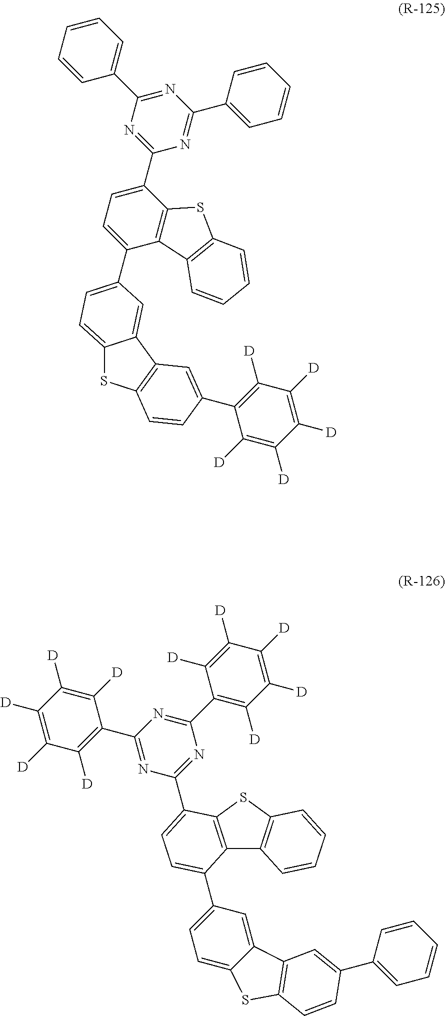 [SMILES: [2H]c1c([2H])c([2H])c(-c2ccc3sc4ccc(-c5ccc(-c6nc(-c7ccccc7)nc(-c7ccccc7)n6)c6sc7ccccc7c56)cc4c3c2)c([2H])c1[2H] ; [2H]c1c([2H])c([2H])c(-c2nc(-c3c([2H])c([2H])c([2H])c([2H])c3[2H])nc(-c3ccc(-c4ccc5sc6ccc(-c7ccccc7)cc6c5c4)c4c3sc3ccccc34)n2)c([2H])c1[2H]]

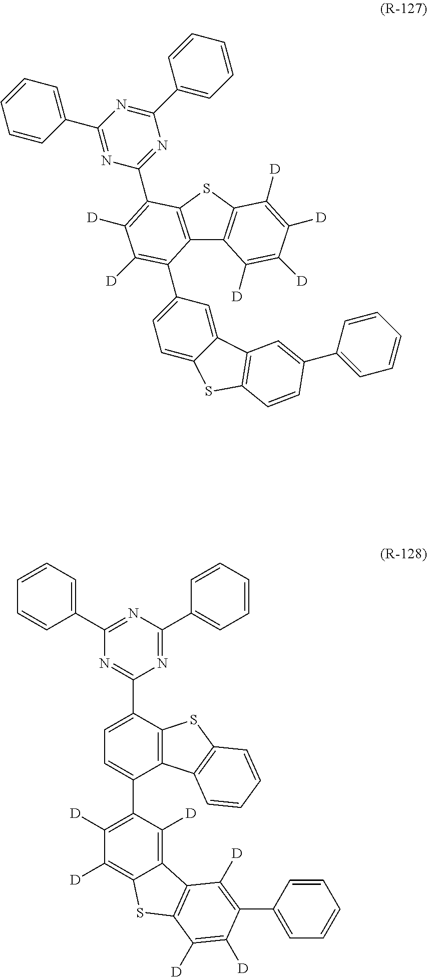 [SMILES: [2H]c1c([2H])c([2H])c2c(sc3c(-c4nc(-c5ccccc5)nc(-c5ccccc5)n4)c([2H])c([2H])c(-c4ccc5sc6ccc(-c7ccccc7)cc6c5c4)c32)c1[2H] ; [2H]c1c(-c2ccccc2)c([2H])c2c(sc3c([2H])c([2H])c(-c4ccc(-c5nc(-c6ccccc6)nc(-c6ccccc6)n5)c5sc6ccccc6c45)c([2H])c32)c1[2H]]

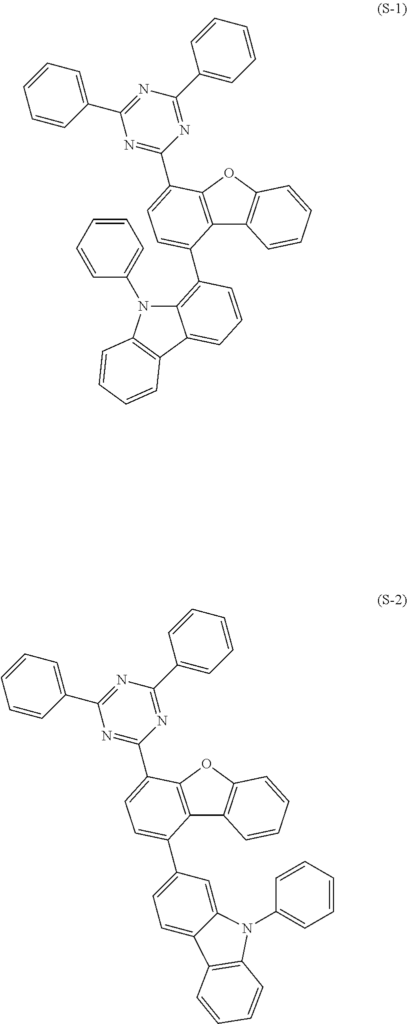 [SMILES: c1ccc(-c2nc(-c3ccccc3)nc(-c3ccc(-c4cccc5c6ccccc6n(-c6ccccc6)c45)c4c3oc3ccccc34)n2)cc1 ; c1ccc(-c2nc(-c3ccccc3)nc(-c3ccc(-c4ccc5c6ccccc6n(-c6ccccc6)c5c4)c4c3oc3ccccc34)n2)cc1]

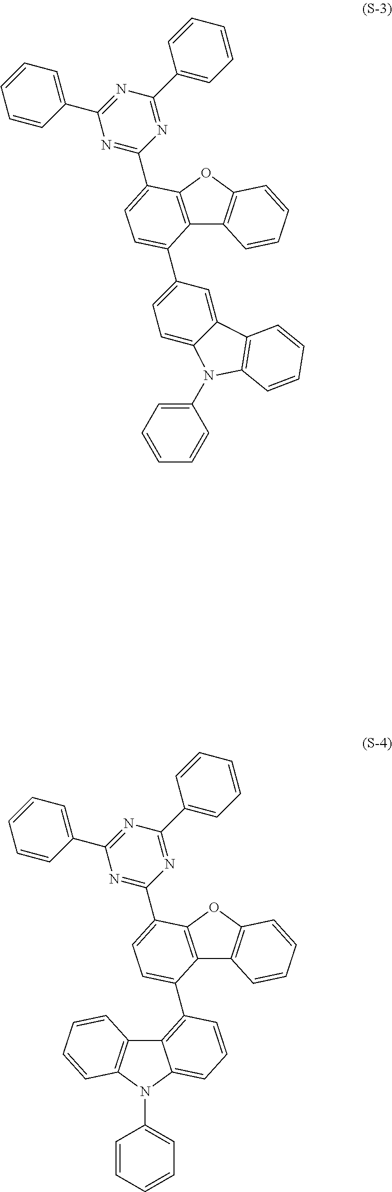 [SMILES: c1ccc(-c2nc(-c3ccccc3)nc(-c3ccc(-c4ccc5c(c4)c4ccccc4n5-c4ccccc4)c4c3oc3ccccc34)n2)cc1 ; c1ccc(-c2nc(-c3ccccc3)nc(-c3ccc(-c4cccc5c4c4ccccc4n5-c4ccccc4)c4c3oc3ccccc34)n2)cc1]

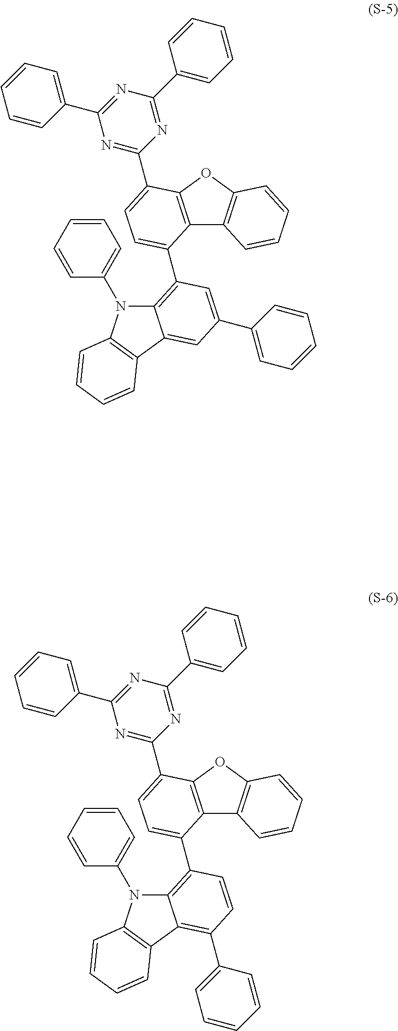 [SMILES: c1ccc(-c2cc(-c3ccc(-c4nc(-c5ccccc5)nc(-c5ccccc5)n4)c4oc5ccccc5c34)c3c(c2)c2ccccc2n3-c2ccccc2)cc1 ; c1ccc(-c2nc(-c3ccccc3)nc(-c3ccc(-c4ccc(-c5ccccc5)c5c6ccccc6n(-c6ccccc6)c45)c4c3oc3ccccc34)n2)cc1]

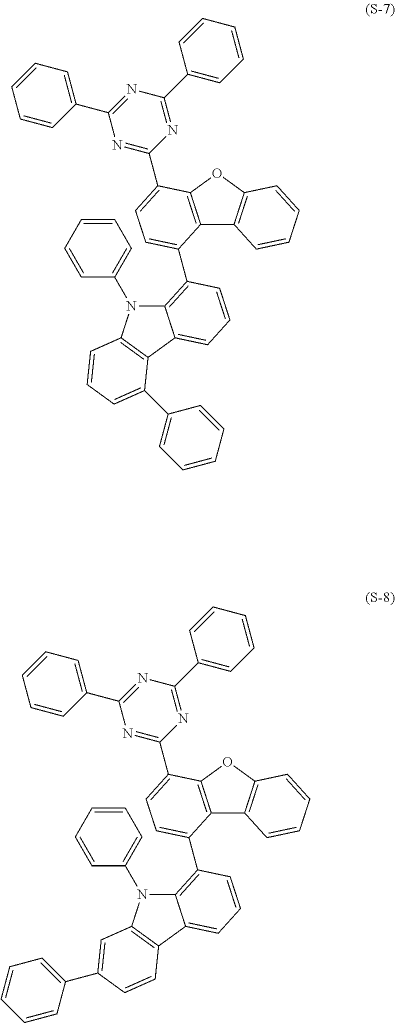 [SMILES: c1ccc(-c2nc(-c3ccccc3)nc(-c3ccc(-c4cccc5c6c(-c7ccccc7)cccc6n(-c6ccccc6)c45)c4c3oc3ccccc34)n2)cc1 ; c1ccc(-c2ccc3c4cccc(-c5ccc(-c6nc(-c7ccccc7)nc(-c7ccccc7)n6)c6oc7ccccc7c56)c4n(-c4ccccc4)c3c2)cc1]

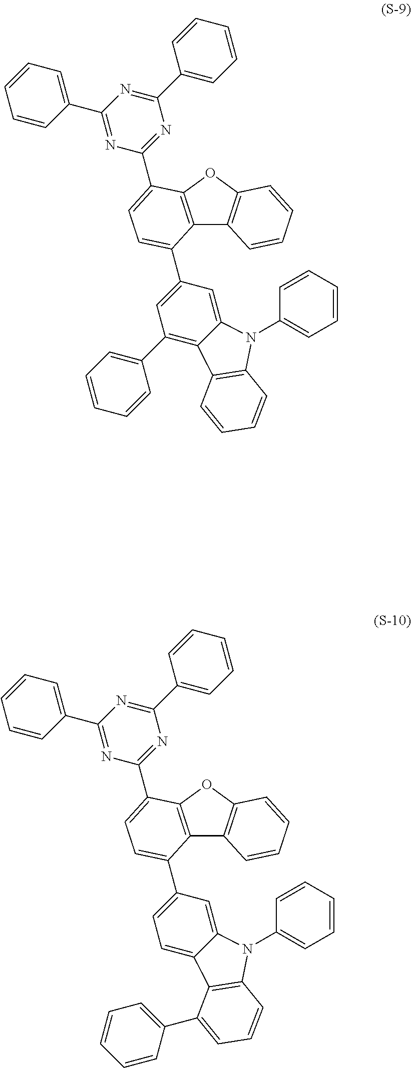 [SMILES: c1ccc(-c2nc(-c3ccccc3)nc(-c3ccc(-c4cc(-c5ccccc5)c5c6ccccc6n(-c6ccccc6)c5c4)c4c3oc3ccccc34)n2)cc1 ; c1ccc(-c2nc(-c3ccccc3)nc(-c3ccc(-c4ccc5c6c(-c7ccccc7)cccc6n(-c6ccccc6)c5c4)c4c3oc3ccccc34)n2)cc1]

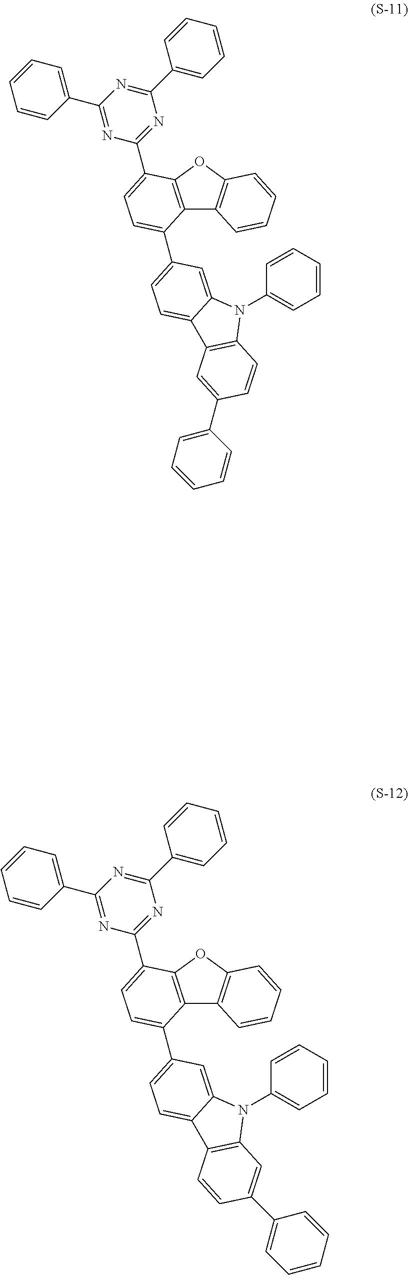 [SMILES: c1ccc(-c2ccc3c(c2)c2ccc(-c4ccc(-c5nc(-c6ccccc6)nc(-c6ccccc6)n5)c5oc6ccccc6c45)cc2n3-c2ccccc2)cc1 ; c1ccc(-c2ccc3c4ccc(-c5ccc(-c6nc(-c7ccccc7)nc(-c7ccccc7)n6)c6oc7ccccc7c56)cc4n(-c4ccccc4)c3c2)cc1]

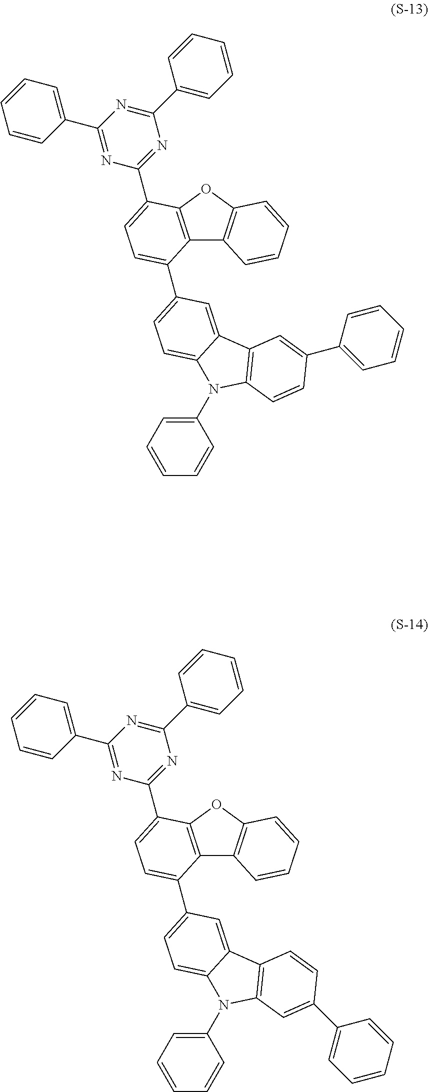 [SMILES: c1ccc(-c2ccc3c(c2)c2cc(-c4ccc(-c5nc(-c6ccccc6)nc(-c6ccccc6)n5)c5oc6ccccc6c45)ccc2n3-c2ccccc2)cc1 ; c1ccc(-c2ccc3c4cc(-c5ccc(-c6nc(-c7ccccc7)nc(-c7ccccc7)n6)c6oc7ccccc7c56)ccc4n(-c4ccccc4)c3c2)cc1]

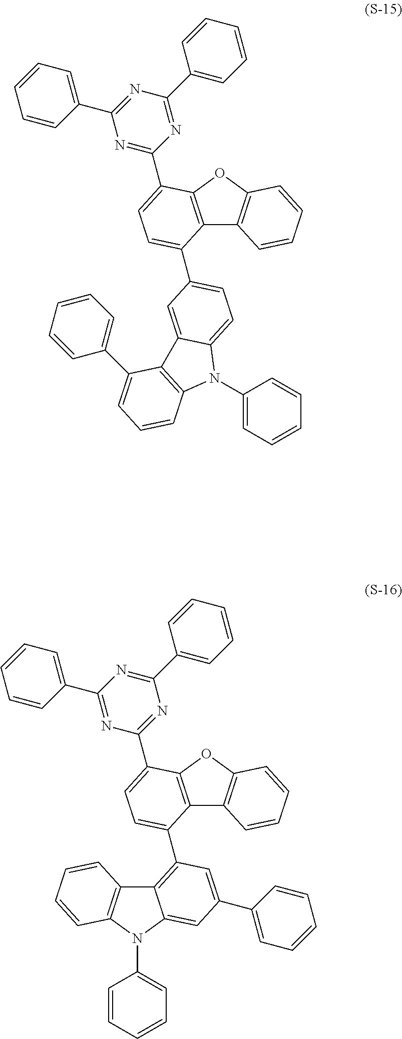 [SMILES: c1ccc(-c2nc(-c3ccccc3)nc(-c3ccc(-c4ccc5c(c4)c4c(-c6ccccc6)cccc4n5-c4ccccc4)c4c3oc3ccccc34)n2)cc1 ; c1ccc(-c2cc(-c3ccc(-c4nc(-c5ccccc5)nc(-c5ccccc5)n4)c4oc5ccccc5c34)c3c4ccccc4n(-c4ccccc4)c3c2)cc1]

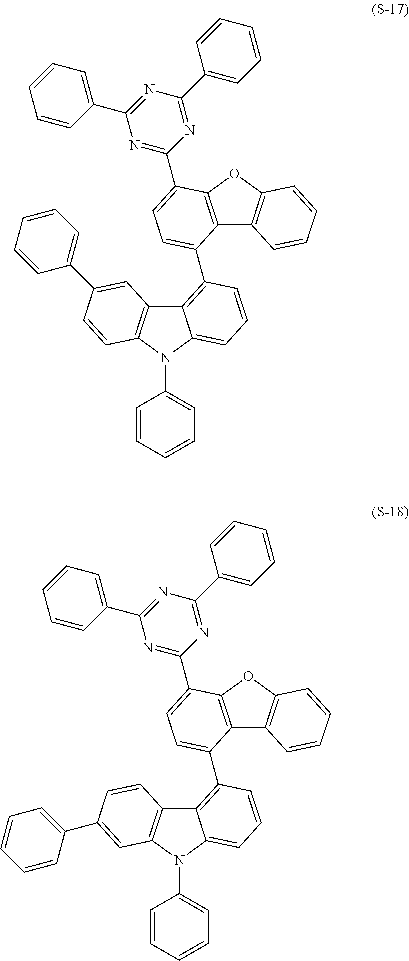 [SMILES: c1ccc(-c2ccc3c(c2)c2c(-c4ccc(-c5nc(-c6ccccc6)nc(-c6ccccc6)n5)c5oc6ccccc6c45)cccc2n3-c2ccccc2)cc1 ; c1ccc(-c2ccc3c4c(-c5ccc(-c6nc(-c7ccccc7)nc(-c7ccccc7)n6)c6oc7ccccc7c56)cccc4n(-c4ccccc4)c3c2)cc1]

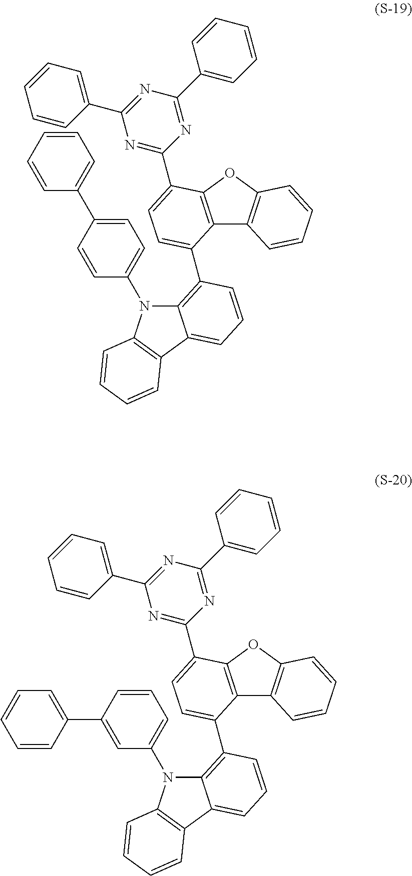 [SMILES: c1ccc(-c2ccc(-n3c4ccccc4c4cccc(-c5ccc(-c6nc(-c7ccccc7)nc(-c7ccccc7)n6)c6oc7ccccc7c56)c43)cc2)cc1 ; c1ccc(-c2cccc(-n3c4ccccc4c4cccc(-c5ccc(-c6nc(-c7ccccc7)nc(-c7ccccc7)n6)c6oc7ccccc7c56)c43)c2)cc1]

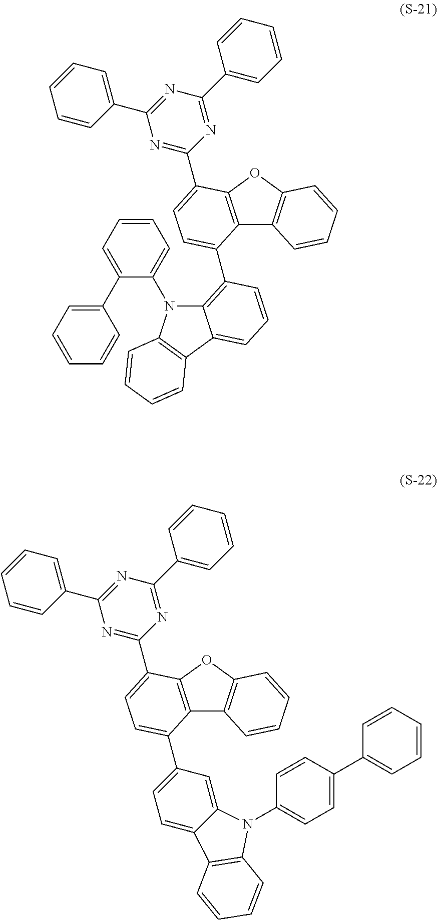 [SMILES: c1ccc(-c2nc(-c3ccccc3)nc(-c3ccc(-c4cccc5c6ccccc6n(-c6ccccc6-c6ccccc6)c45)c4c3oc3ccccc34)n2)cc1 ; c1ccc(-c2ccc(-n3c4ccccc4c4ccc(-c5ccc(-c6nc(-c7ccccc7)nc(-c7ccccc7)n6)c6oc7ccccc7c56)cc43)cc2)cc1]

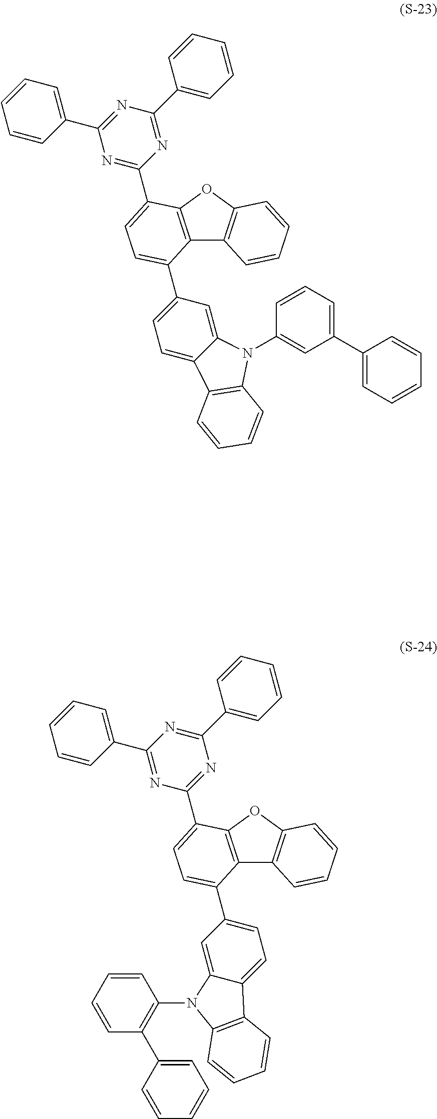 [SMILES: c1ccc(-c2cccc(-n3c4ccccc4c4ccc(-c5ccc(-c6nc(-c7ccccc7)nc(-c7ccccc7)n6)c6oc7ccccc7c56)cc43)c2)cc1 ; c1ccc(-c2nc(-c3ccccc3)nc(-c3ccc(-c4ccc5c6ccccc6n(-c6ccccc6-c6ccccc6)c5c4)c4c3oc3ccccc34)n2)cc1]

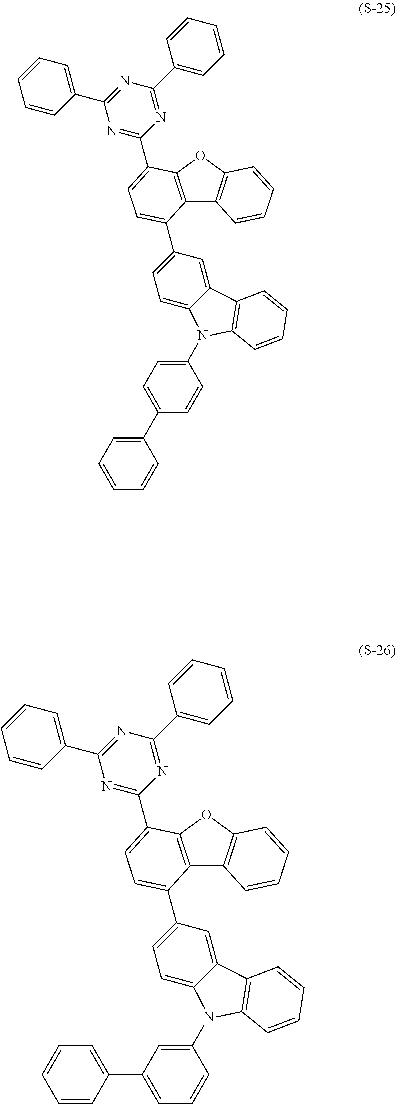 [SMILES: c1ccc(-c2ccc(-n3c4ccccc4c4cc(-c5ccc(-c6nc(-c7ccccc7)nc(-c7ccccc7)n6)c6oc7ccccc7c56)ccc43)cc2)cc1 ; c1ccc(-c2cccc(-n3c4ccccc4c4cc(-c5ccc(-c6nc(-c7ccccc7)nc(-c7ccccc7)n6)c6oc7ccccc7c56)ccc43)c2)cc1]

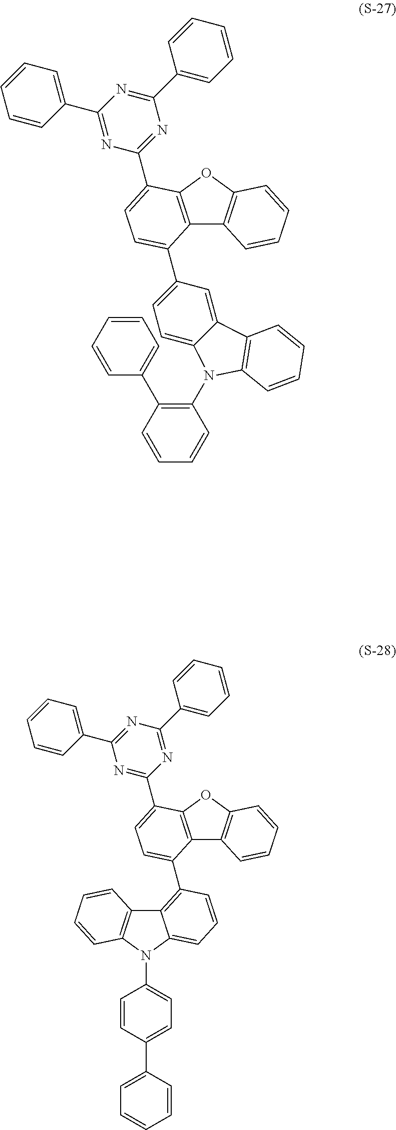 [SMILES: c1ccc(-c2nc(-c3ccccc3)nc(-c3ccc(-c4ccc5c(c4)c4ccccc4n5-c4ccccc4-c4ccccc4)c4c3oc3ccccc34)n2)cc1 ; c1ccc(-c2ccc(-n3c4ccccc4c4c(-c5ccc(-c6nc(-c7ccccc7)nc(-c7ccccc7)n6)c6oc7ccccc7c56)cccc43)cc2)cc1]

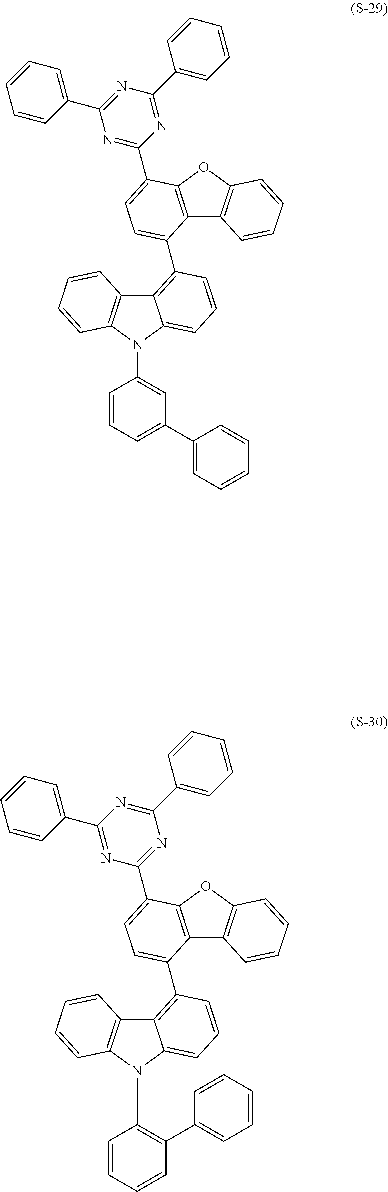 [SMILES: c1ccc(-c2cccc(-n3c4ccccc4c4c(-c5ccc(-c6nc(-c7ccccc7)nc(-c7ccccc7)n6)c6oc7ccccc7c56)cccc43)c2)cc1 ; c1ccc(-c2nc(-c3ccccc3)nc(-c3ccc(-c4cccc5c4c4ccccc4n5-c4ccccc4-c4ccccc4)c4c3oc3ccccc34)n2)cc1]

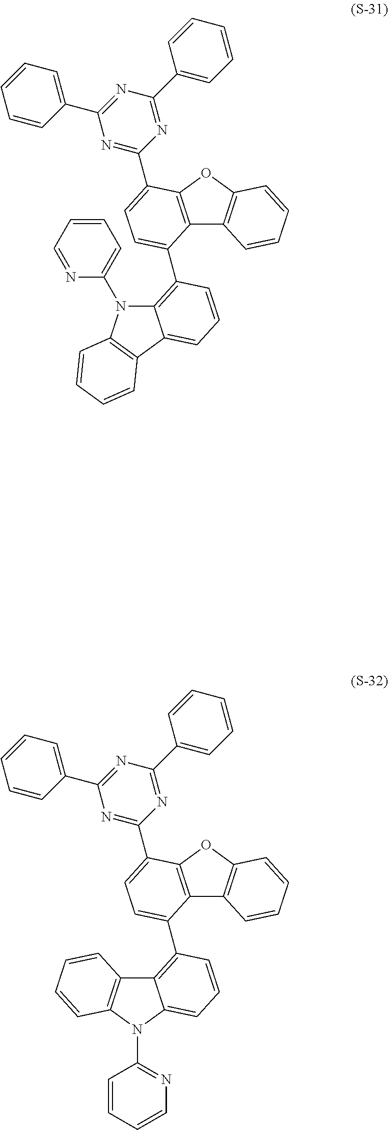 [SMILES: c1ccc(-c2nc(-c3ccccc3)nc(-c3ccc(-c4cccc5c6ccccc6n(-c6ccccn6)c45)c4c3oc3ccccc34)n2)cc1 ; c1ccc(-c2nc(-c3ccccc3)nc(-c3ccc(-c4cccc5c4c4ccccc4n5-c4ccccn4)c4c3oc3ccccc34)n2)cc1]

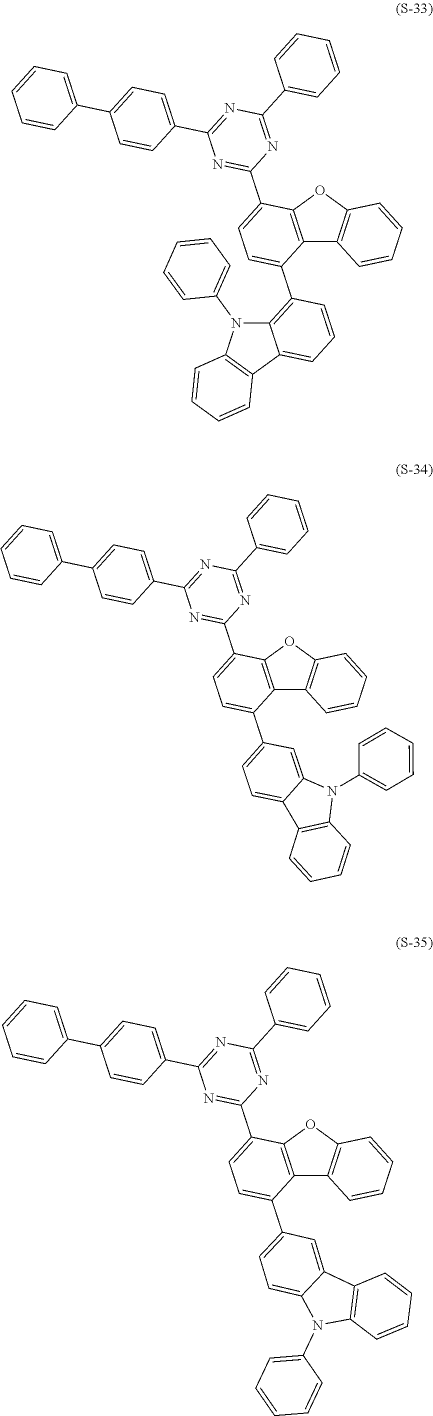 [SMILES: c1ccc(-c2ccc(-c3nc(-c4ccccc4)nc(-c4ccc(-c5cccc6c7ccccc7n(-c7ccccc7)c56)c5c4oc4ccccc45)n3)cc2)cc1 ; c1ccc(-c2ccc(-c3nc(-c4ccccc4)nc(-c4ccc(-c5ccc6c7ccccc7n(-c7ccccc7)c6c5)c5c4oc4ccccc45)n3)cc2)cc1 ; c1ccc(-c2ccc(-c3nc(-c4ccccc4)nc(-c4ccc(-c5ccc6c(c5)c5ccccc5n6-c5ccccc5)c5c4oc4ccccc45)n3)cc2)cc1]

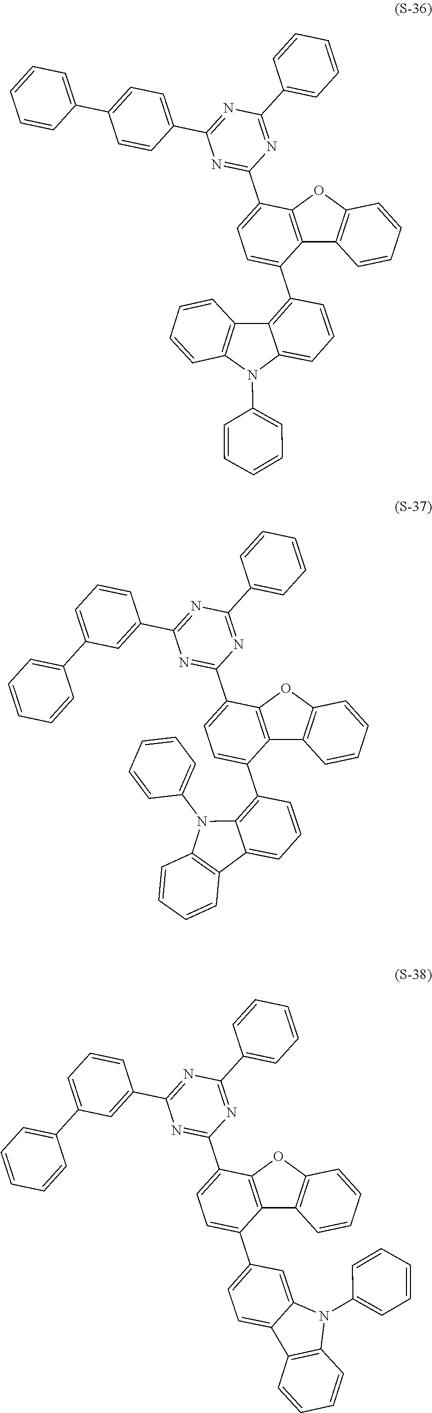 [SMILES: c1ccc(-c2ccc(-c3nc(-c4ccccc4)nc(-c4ccc(-c5cccc6c5c5ccccc5n6-c5ccccc5)c5c4oc4ccccc45)n3)cc2)cc1 ; c1ccc(-c2cccc(-c3nc(-c4ccccc4)nc(-c4ccc(-c5cccc6c7ccccc7n(-c7ccccc7)c56)c5c4oc4ccccc45)n3)c2)cc1 ; c1ccc(-c2cccc(-c3nc(-c4ccccc4)nc(-c4ccc(-c5ccc6c7ccccc7n(-c7ccccc7)c6c5)c5c4oc4ccccc45)n3)c2)cc1]

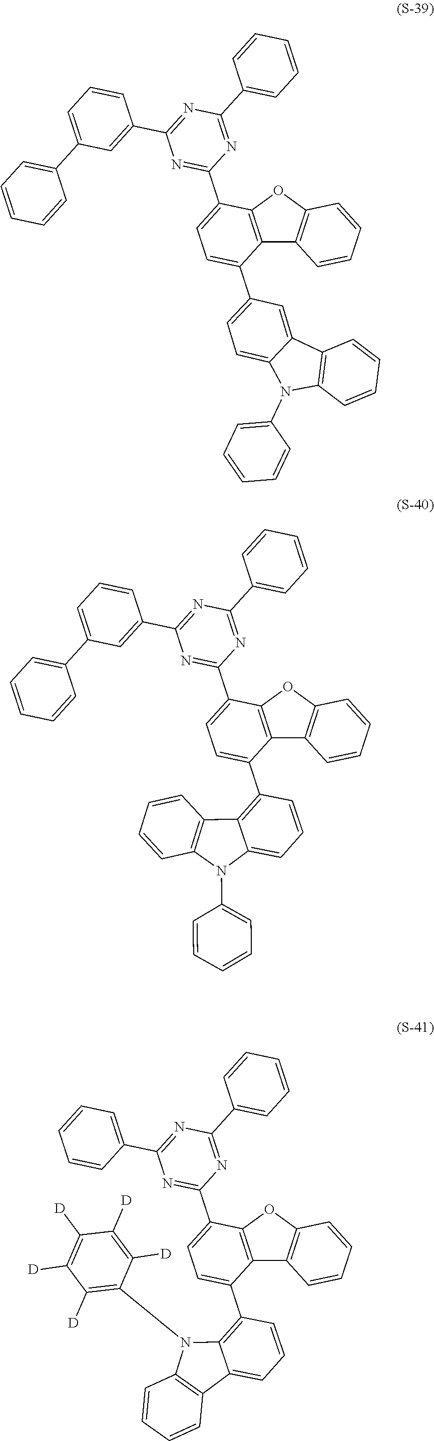 [SMILES: c1ccc(-c2cccc(-c3nc(-c4ccccc4)nc(-c4ccc(-c5ccc6c(c5)c5ccccc5n6-c5ccccc5)c5c4oc4ccccc45)n3)c2)cc1 ; c1ccc(-c2cccc(-c3nc(-c4ccccc4)nc(-c4ccc(-c5cccc6c5c5ccccc5n6-c5ccccc5)c5c4oc4ccccc45)n3)c2)cc1 ; [2H]c1c([2H])c([2H])c(-n2c3ccccc3c3cccc(-c4ccc(-c5nc(-c6ccccc6)nc(-c6ccccc6)n5)c5oc6ccccc6c45)c32)c([2H])c1[2H]]

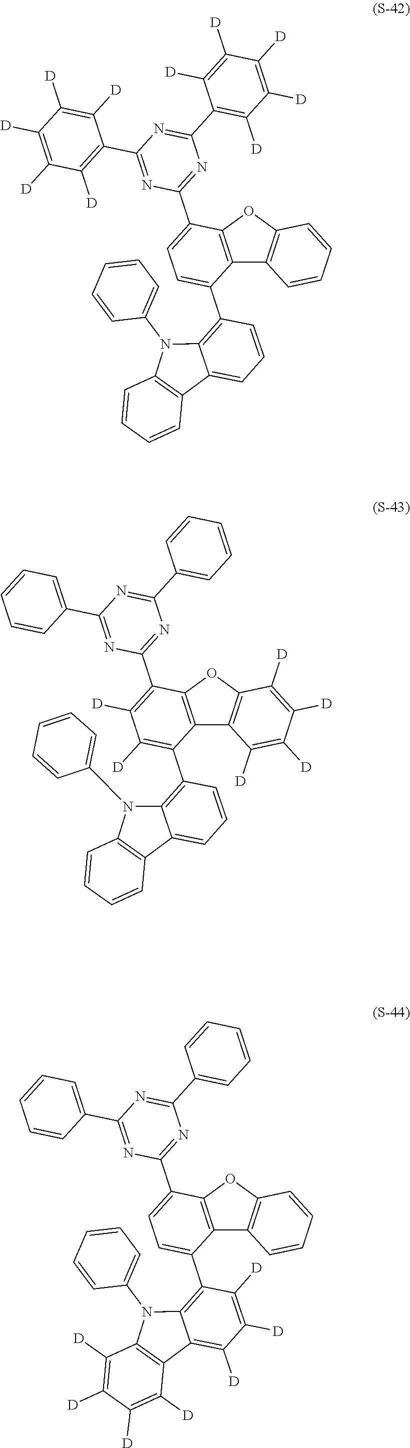 [SMILES: [2H]c1c([2H])c([2H])c(-c2nc(-c3c([2H])c([2H])c([2H])c([2H])c3[2H])nc(-c3ccc(-c4cccc5c6ccccc6n(-c6ccccc6)c45)c4c3oc3ccccc34)n2)c([2H])c1[2H] ; [2H]c1c([2H])c([2H])c2c(oc3c(-c4nc(-c5ccccc5)nc(-c5ccccc5)n4)c([2H])c([2H])c(-c4cccc5c6ccccc6n(-c6ccccc6)c45)c32)c1[2H] ; [2H]c1c([2H])c([2H])c2c(c1[2H])c1c([2H])c([2H])c([2H])c(-c3ccc(-c4nc(-c5ccccc5)nc(-c5ccccc5)n4)c4oc5ccccc5c34)c1n2-c1ccccc1]

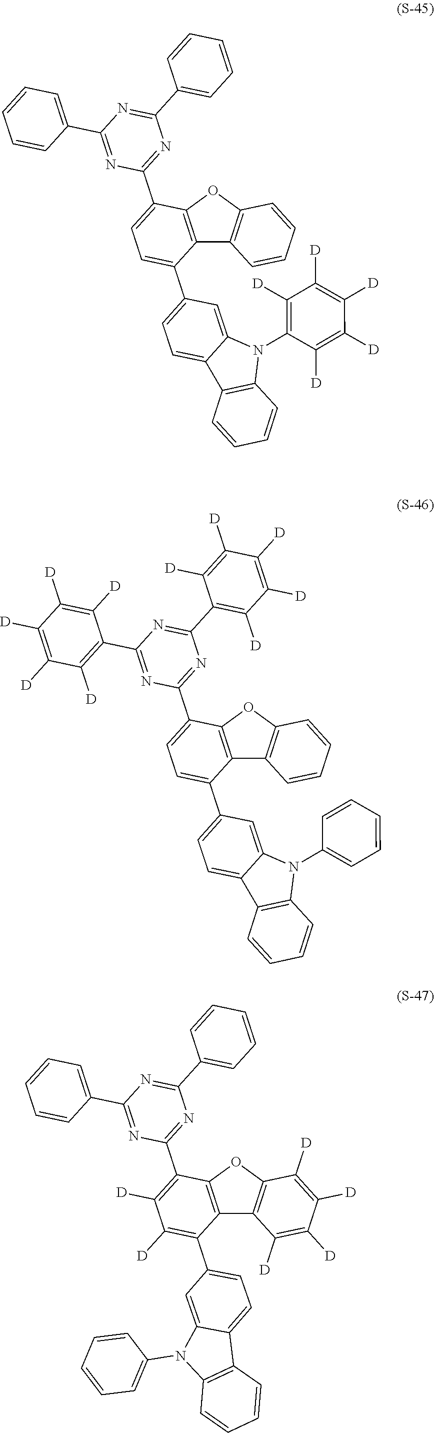 [SMILES: [2H]c1c([2H])c([2H])c(-n2c3ccccc3c3ccc(-c4ccc(-c5nc(-c6ccccc6)nc(-c6ccccc6)n5)c5oc6ccccc6c45)cc32)c([2H])c1[2H] ; [2H]c1c([2H])c([2H])c(-c2nc(-c3c([2H])c([2H])c([2H])c([2H])c3[2H])nc(-c3ccc(-c4ccc5c6ccccc6n(-c6ccccc6)c5c4)c4c3oc3ccccc34)n2)c([2H])c1[2H] ; [2H]c1c([2H])c([2H])c2c(oc3c(-c4nc(-c5ccccc5)nc(-c5ccccc5)n4)c([2H])c([2H])c(-c4ccc5c6ccccc6n(-c6ccccc6)c5c4)c32)c1[2H]]

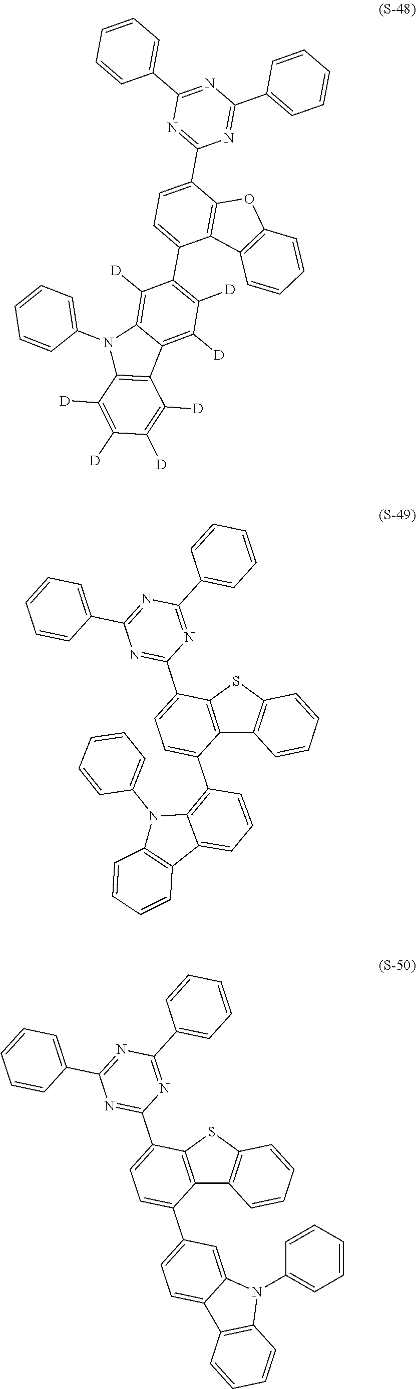 [SMILES: [2H]c1c([2H])c([2H])c2c(c1[2H])c1c([2H])c([2H])c(-c3ccc(-c4nc(-c5ccccc5)nc(-c5ccccc5)n4)c4oc5ccccc5c34)c([2H])c1n2-c1ccccc1 ; c1ccc(-c2nc(-c3ccccc3)nc(-c3ccc(-c4cccc5c6ccccc6n(-c6ccccc6)c45)c4c3sc3ccccc34)n2)cc1 ; c1ccc(-c2nc(-c3ccccc3)nc(-c3ccc(-c4ccc5c6ccccc6n(-c6ccccc6)c5c4)c4c3sc3ccccc34)n2)cc1]

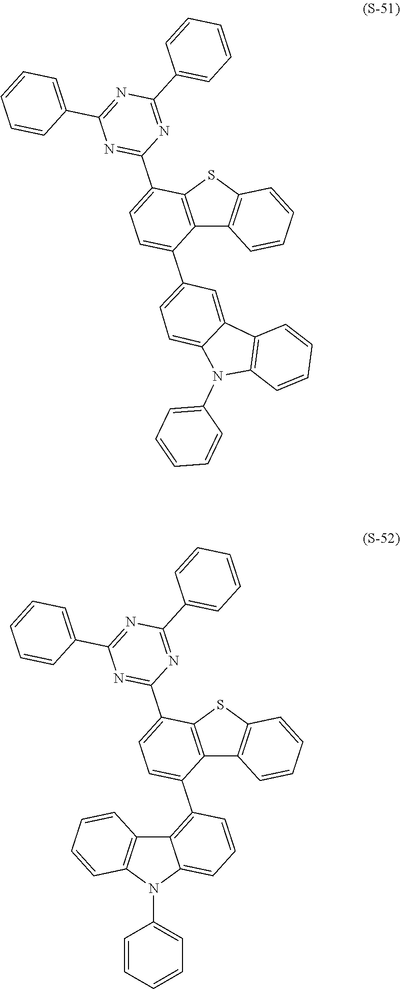 [SMILES: c1ccc(-c2nc(-c3ccccc3)nc(-c3ccc(-c4ccc5c(c4)c4ccccc4n5-c4ccccc4)c4c3sc3ccccc34)n2)cc1 ; c1ccc(-c2nc(-c3ccccc3)nc(-c3ccc(-c4cccc5c4c4ccccc4n5-c4ccccc4)c4c3sc3ccccc34)n2)cc1]

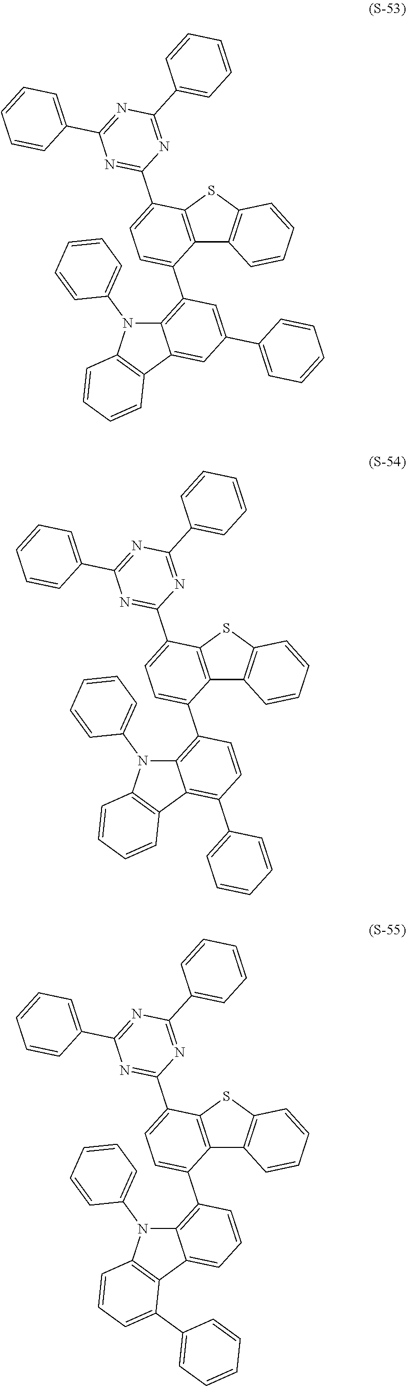 [SMILES: c1ccc(-c2cc(-c3ccc(-c4nc(-c5ccccc5)nc(-c5ccccc5)n4)c4sc5ccccc5c34)c3c(c2)c2ccccc2n3-c2ccccc2)cc1 ; c1ccc(-c2nc(-c3ccccc3)nc(-c3ccc(-c4ccc(-c5ccccc5)c5c6ccccc6n(-c6ccccc6)c45)c4c3sc3ccccc34)n2)cc1 ; c1ccc(-c2nc(-c3ccccc3)nc(-c3ccc(-c4cccc5c6c(-c7ccccc7)cccc6n(-c6ccccc6)c45)c4c3sc3ccccc34)n2)cc1]

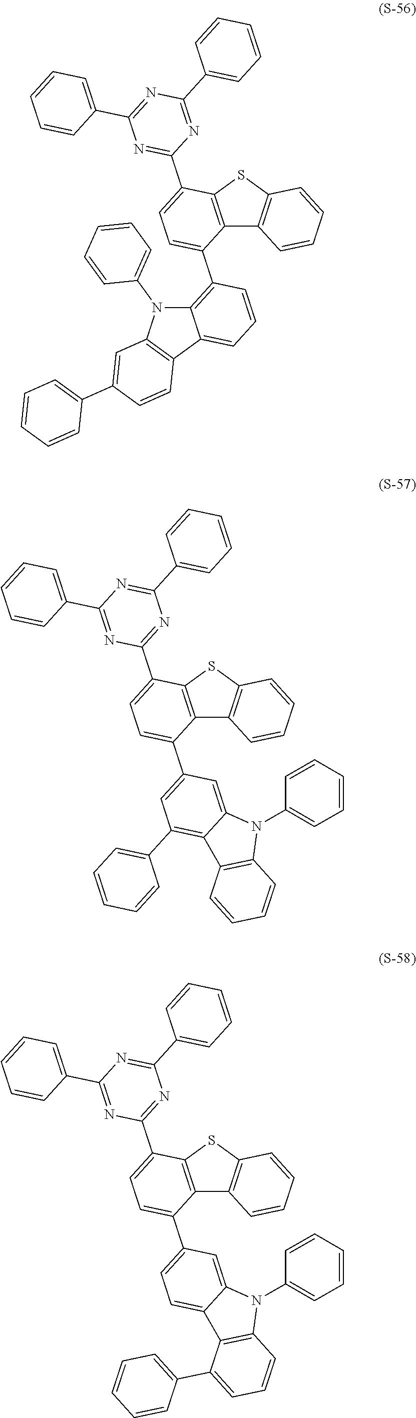 [SMILES: c1ccc(-c2ccc3c4cccc(-c5ccc(-c6nc(-c7ccccc7)nc(-c7ccccc7)n6)c6sc7ccccc7c56)c4n(-c4ccccc4)c3c2)cc1 ; c1ccc(-c2nc(-c3ccccc3)nc(-c3ccc(-c4cc(-c5ccccc5)c5c6ccccc6n(-c6ccccc6)c5c4)c4c3sc3ccccc34)n2)cc1 ; c1ccc(-c2nc(-c3ccccc3)nc(-c3ccc(-c4ccc5c6c(-c7ccccc7)cccc6n(-c6ccccc6)c5c4)c4c3sc3ccccc34)n2)cc1]

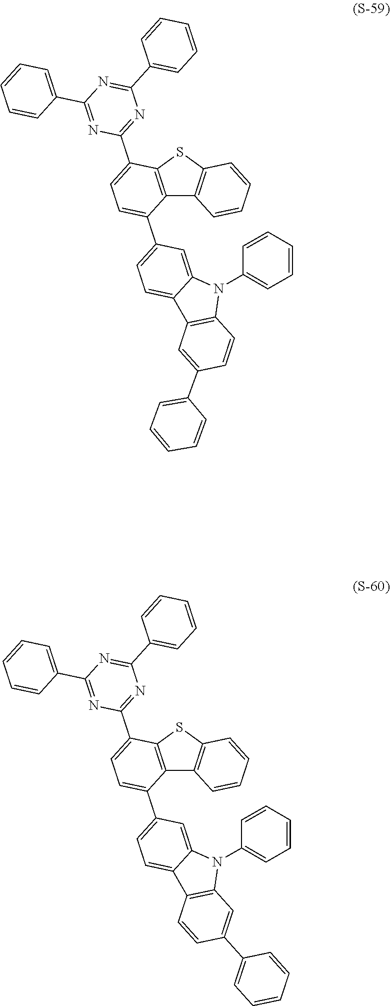 [SMILES: c1ccc(-c2ccc3c(c2)c2ccc(-c4ccc(-c5nc(-c6ccccc6)nc(-c6ccccc6)n5)c5sc6ccccc6c45)cc2n3-c2ccccc2)cc1 ; c1ccc(-c2ccc3c4ccc(-c5ccc(-c6nc(-c7ccccc7)nc(-c7ccccc7)n6)c6sc7ccccc7c56)cc4n(-c4ccccc4)c3c2)cc1]

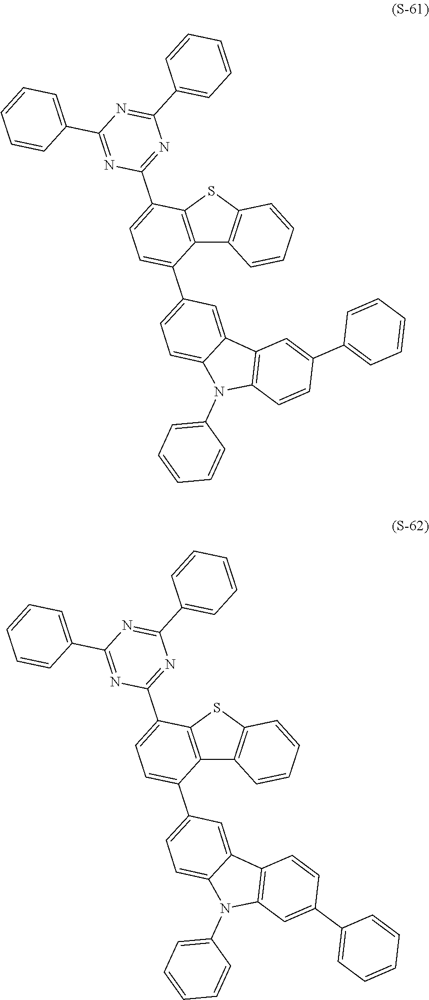 [SMILES: c1ccc(-c2ccc3c(c2)c2cc(-c4ccc(-c5nc(-c6ccccc6)nc(-c6ccccc6)n5)c5sc6ccccc6c45)ccc2n3-c2ccccc2)cc1 ; c1ccc(-c2ccc3c4cc(-c5ccc(-c6nc(-c7ccccc7)nc(-c7ccccc7)n6)c6sc7ccccc7c56)ccc4n(-c4ccccc4)c3c2)cc1]

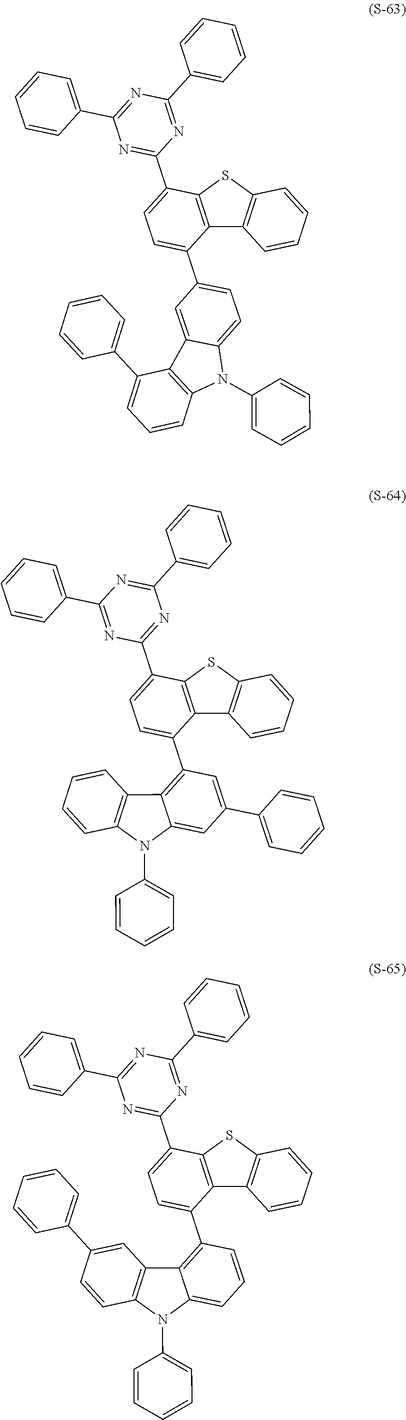 [SMILES: c1ccc(-c2nc(-c3ccccc3)nc(-c3ccc(-c4ccc5c(c4)c4c(-c6ccccc6)cccc4n5-c4ccccc4)c4c3sc3ccccc34)n2)cc1 ; c1ccc(-c2cc(-c3ccc(-c4nc(-c5ccccc5)nc(-c5ccccc5)n4)c4sc5ccccc5c34)c3c4ccccc4n(-c4ccccc4)c3c2)cc1 ; c1ccc(-c2ccc3c(c2)c2c(-c4ccc(-c5nc(-c6ccccc6)nc(-c6ccccc6)n5)c5sc6ccccc6c45)cccc2n3-c2ccccc2)cc1]

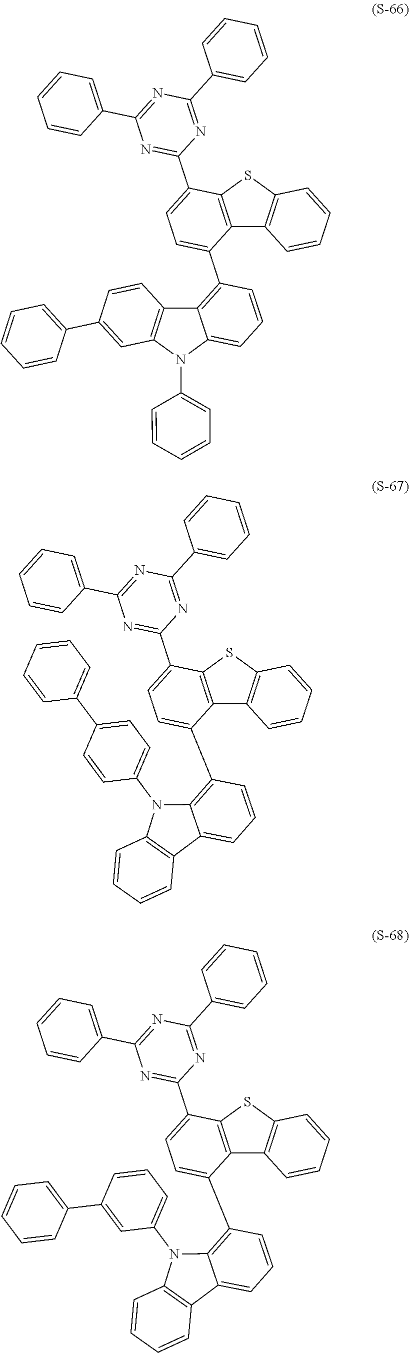

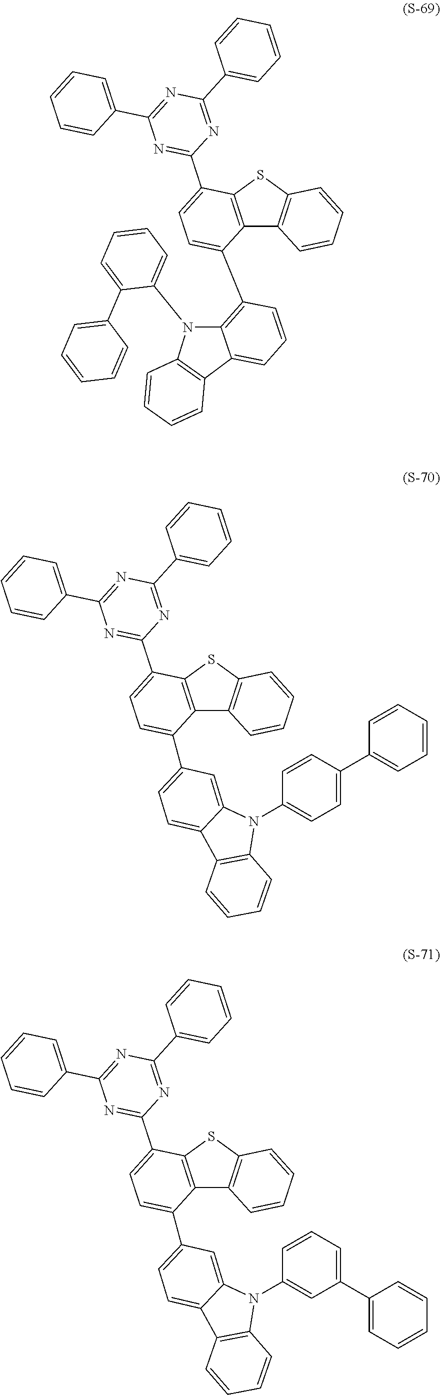 [SMILES: c1ccc(-c2nc(-c3ccccc3)nc(-c3ccc(-c4cccc5c6ccccc6n(-c6ccccc6-c6ccccc6)c45)c4c3sc3ccccc34)n2)cc1 ; c1ccc(-c2ccc(-n3c4ccccc4c4ccc(-c5ccc(-c6nc(-c7ccccc7)nc(-c7ccccc7)n6)c6sc7ccccc7c56)cc43)cc2)cc1 ; c1ccc(-c2cccc(-n3c4ccccc4c4ccc(-c5ccc(-c6nc(-c7ccccc7)nc(-c7ccccc7)n6)c6sc7ccccc7c56)cc43)c2)cc1]

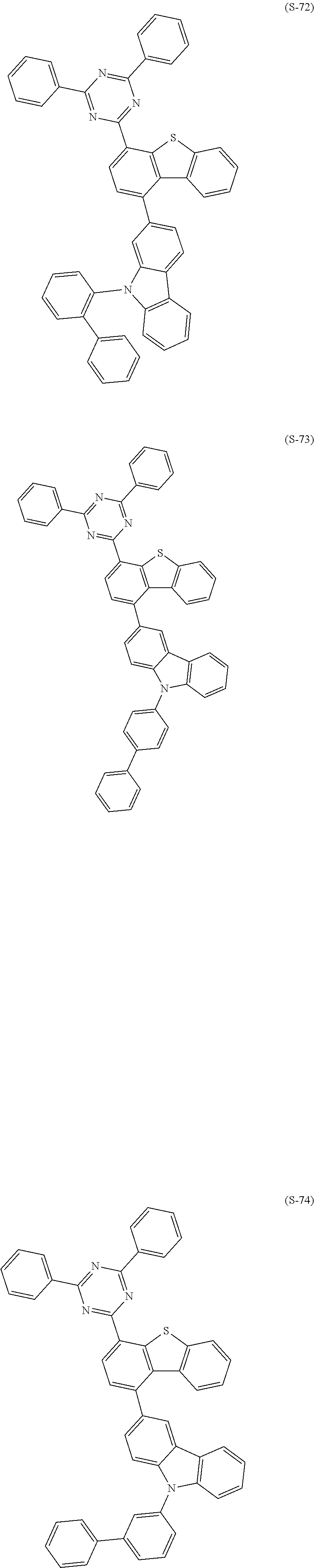 [SMILES: c1ccc(-c2nc(-c3ccccc3)nc(-c3ccc(-c4ccc5c6ccccc6n(-c6ccccc6-c6ccccc6)c5c4)c4c3sc3ccccc34)n2)cc1 ; c1ccc(-c2ccc(-n3c4ccccc4c4cc(-c5ccc(-c6nc(-c7ccccc7)nc(-c7ccccc7)n6)c6sc7ccccc7c56)ccc43)cc2)cc1 ; c1ccc(-c2cccc(-n3c4ccccc4c4cc(-c5ccc(-c6nc(-c7ccccc7)nc(-c7ccccc7)n6)c6sc7ccccc7c56)ccc43)c2)cc1]

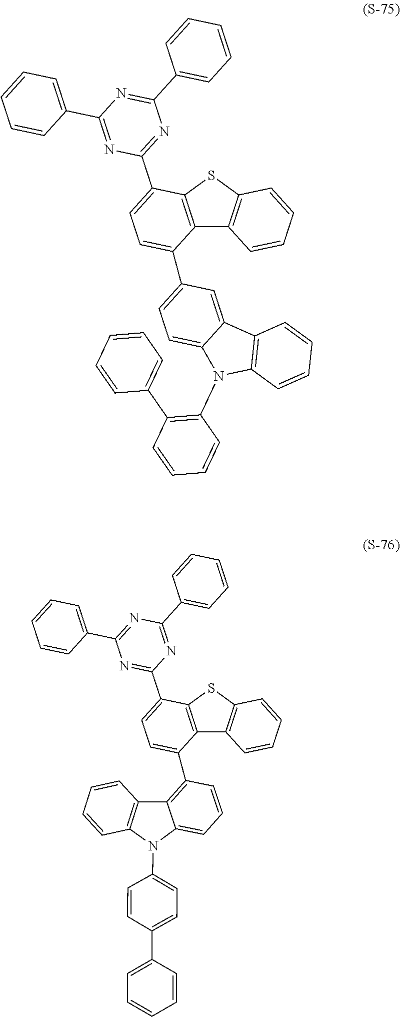 [SMILES: c1ccc(-c2nc(-c3ccccc3)nc(-c3ccc(-c4ccc5c(c4)c4ccccc4n5-c4ccccc4-c4ccccc4)c4c3sc3ccccc34)n2)cc1 ; c1ccc(-c2ccc(-n3c4ccccc4c4c(-c5ccc(-c6nc(-c7ccccc7)nc(-c7ccccc7)n6)c6sc7ccccc7c56)cccc43)cc2)cc1]

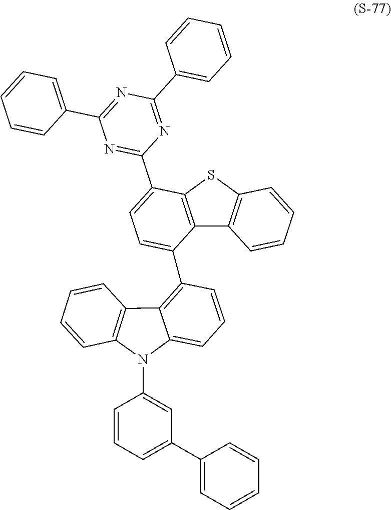 [SMILES: c1ccc(-c2cccc(-n3c4ccccc4c4c(-c5ccc(-c6nc(-c7ccccc7)nc(-c7ccccc7)n6)c6sc7ccccc7c56)cccc43)c2)cc1]

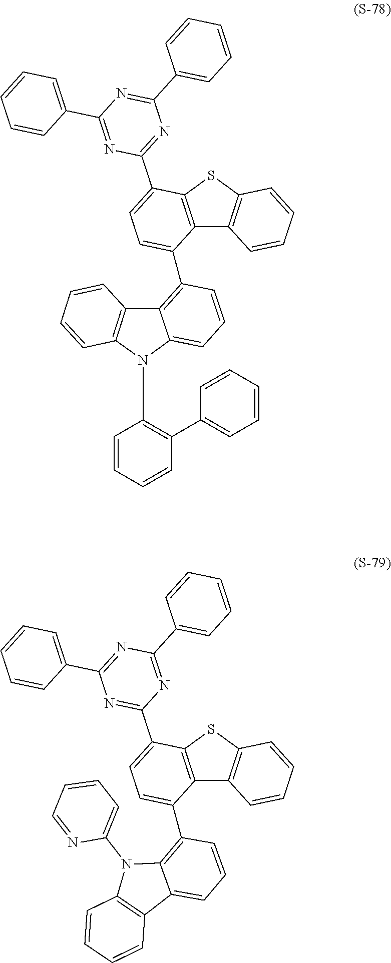 [SMILES: c1ccc(-c2nc(-c3ccccc3)nc(-c3ccc(-c4cccc5c4c4ccccc4n5-c4ccccc4-c4ccccc4)c4c3sc3ccccc34)n2)cc1 ; c1ccc(-c2nc(-c3ccccc3)nc(-c3ccc(-c4cccc5c6ccccc6n(-c6ccccn6)c45)c4c3sc3ccccc34)n2)cc1]

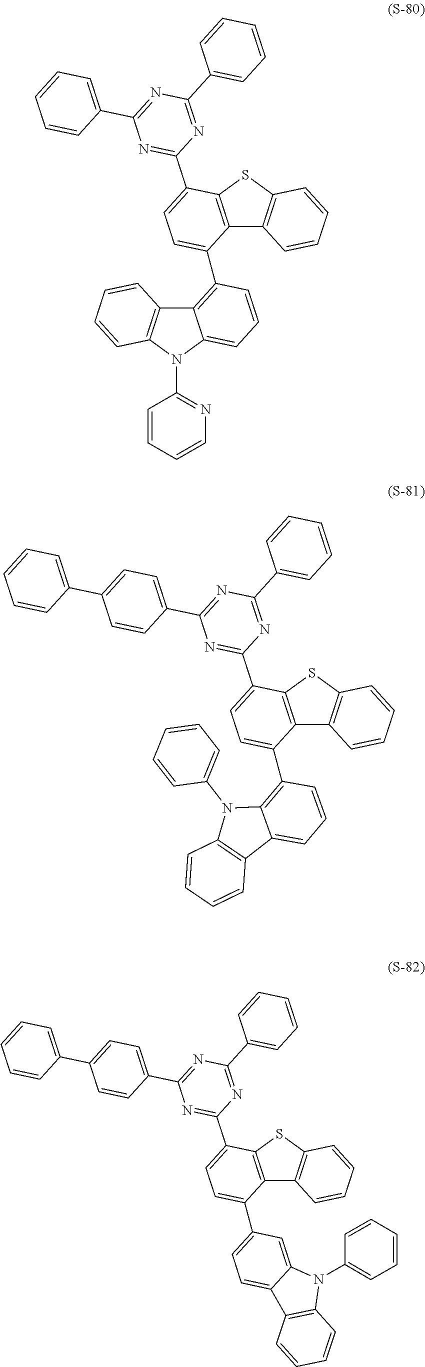 [SMILES: c1ccc(-c2nc(-c3ccccc3)nc(-c3ccc(-c4cccc5c4c4ccccc4n5-c4ccccn4)c4c3sc3ccccc34)n2)cc1 ; c1ccc(-c2ccc(-c3nc(-c4ccccc4)nc(-c4ccc(-c5cccc6c7ccccc7n(-c7ccccc7)c56)c5c4sc4ccccc45)n3)cc2)cc1 ; c1ccc(-c2ccc(-c3nc(-c4ccccc4)nc(-c4ccc(-c5ccc6c7ccccc7n(-c7ccccc7)c6c5)c5c4sc4ccccc45)n3)cc2)cc1]

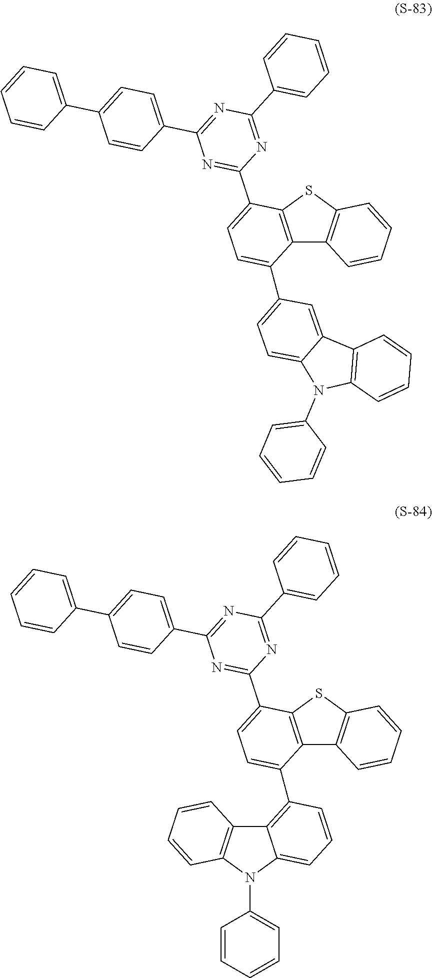 [SMILES: c1ccc(-c2ccc(-c3nc(-c4ccccc4)nc(-c4ccc(-c5ccc6c(c5)c5ccccc5n6-c5ccccc5)c5c4sc4ccccc45)n3)cc2)cc1 ; c1ccc(-c2ccc(-c3nc(-c4ccccc4)nc(-c4ccc(-c5cccc6c5c5ccccc5n6-c5ccccc5)c5c4sc4ccccc45)n3)cc2)cc1]

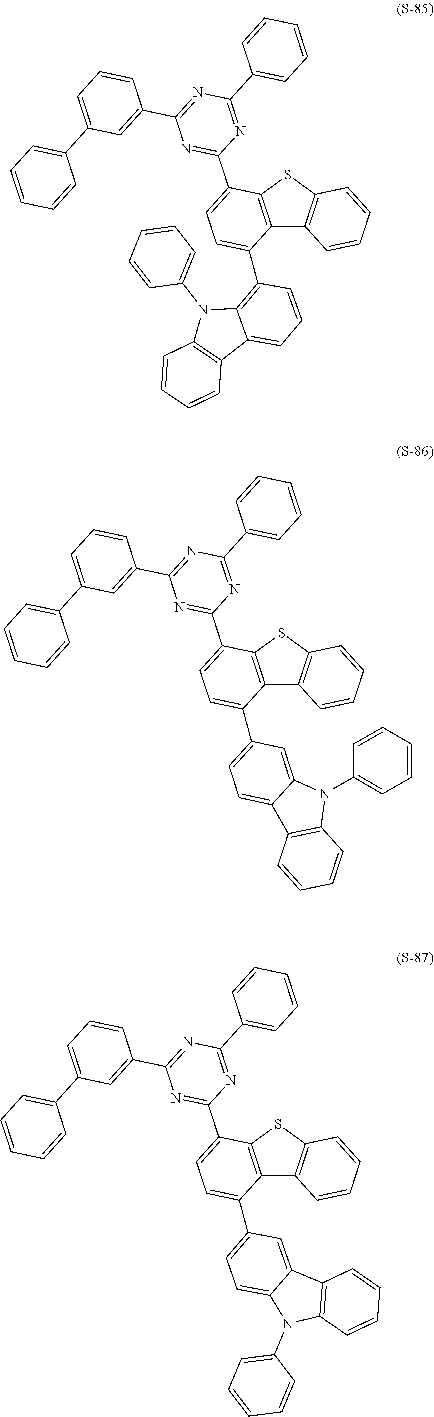 [SMILES: c1ccc(-c2cccc(-c3nc(-c4ccccc4)nc(-c4ccc(-c5cccc6c7ccccc7n(-c7ccccc7)c56)c5c4sc4ccccc45)n3)c2)cc1 ; c1ccc(-c2cccc(-c3nc(-c4ccccc4)nc(-c4ccc(-c5ccc6c7ccccc7n(-c7ccccc7)c6c5)c5c4sc4ccccc45)n3)c2)cc1 ; c1ccc(-c2cccc(-c3nc(-c4ccccc4)nc(-c4ccc(-c5ccc6c(c5)c5ccccc5n6-c5ccccc5)c5c4sc4ccccc45)n3)c2)cc1]

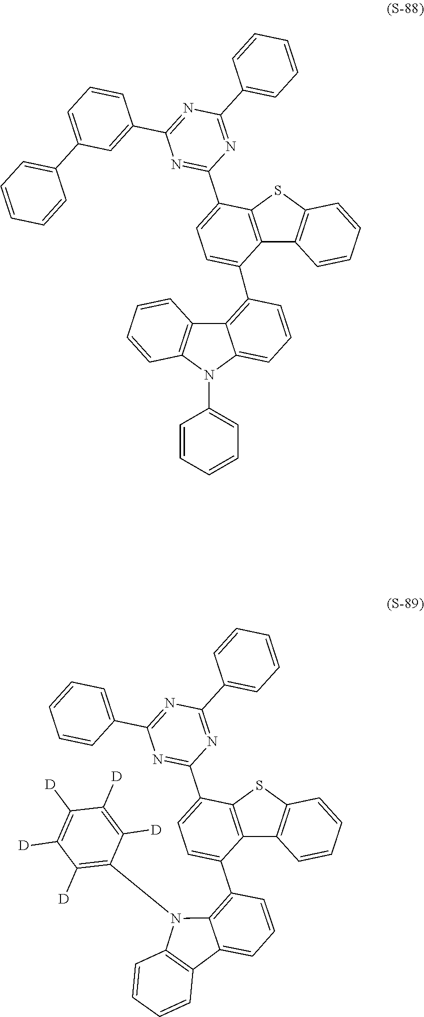 [SMILES: c1ccc(-c2cccc(-c3nc(-c4ccccc4)nc(-c4ccc(-c5cccc6c5c5ccccc5n6-c5ccccc5)c5c4sc4ccccc45)n3)c2)cc1 ; [2H]c1c([2H])c([2H])c(-n2c3ccccc3c3cccc(-c4ccc(-c5nc(-c6ccccc6)nc(-c6ccccc6)n5)c5sc6ccccc6c45)c32)c([2H])c1[2H]]

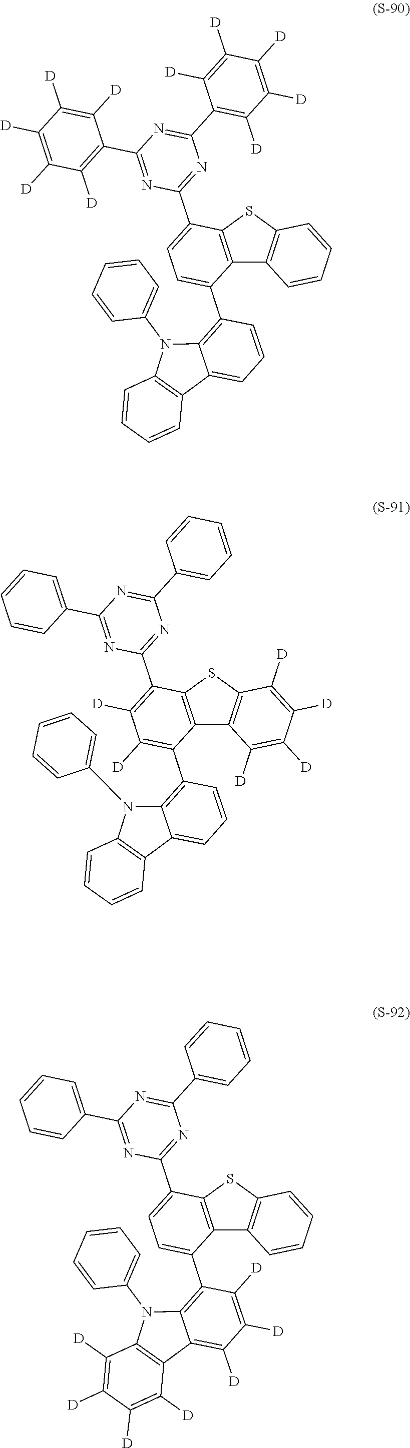 [SMILES: [2H]c1c([2H])c([2H])c(-c2nc(-c3c([2H])c([2H])c([2H])c([2H])c3[2H])nc(-c3ccc(-c4cccc5c6ccccc6n(-c6ccccc6)c45)c4c3sc3ccccc34)n2)c([2H])c1[2H] ; [2H]c1c([2H])c([2H])c2c(sc3c(-c4nc(-c5ccccc5)nc(-c5ccccc5)n4)c([2H])c([2H])c(-c4cccc5c6ccccc6n(-c6ccccc6)c45)c32)c1[2H] ; [2H]c1c([2H])c([2H])c2c(c1[2H])c1c([2H])c([2H])c([2H])c(-c3ccc(-c4nc(-c5ccccc5)nc(-c5ccccc5)n4)c4sc5ccccc5c34)c1n2-c1ccccc1]

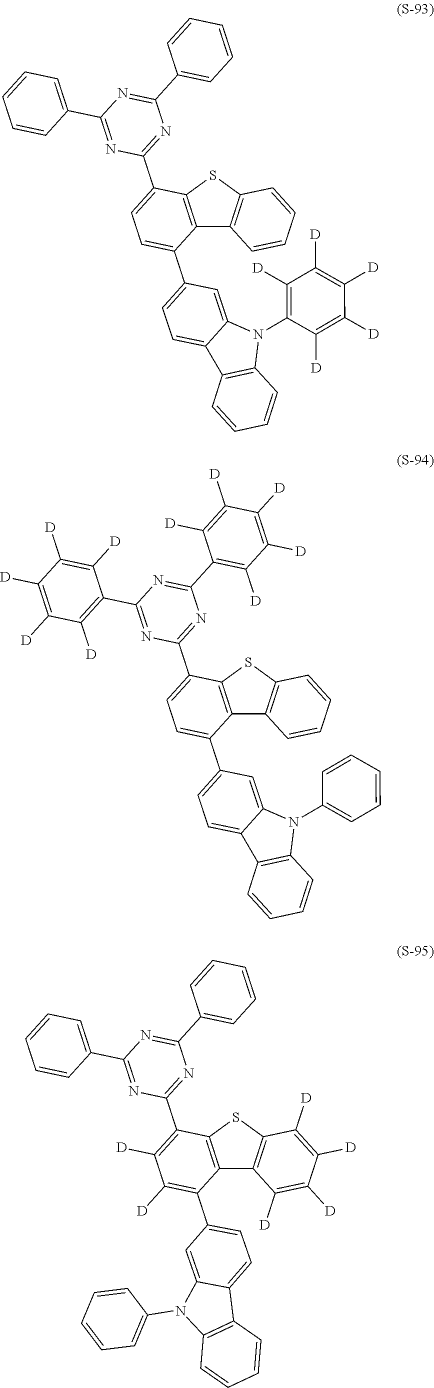 [SMILES: [2H]c1c([2H])c([2H])c(-n2c3ccccc3c3ccc(-c4ccc(-c5nc(-c6ccccc6)nc(-c6ccccc6)n5)c5sc6ccccc6c45)cc32)c([2H])c1[2H] ; [2H]c1c([2H])c([2H])c(-c2nc(-c3c([2H])c([2H])c([2H])c([2H])c3[2H])nc(-c3ccc(-c4ccc5c6ccccc6n(-c6ccccc6)c5c4)c4c3sc3ccccc34)n2)c([2H])c1[2H] ; [2H]c1c([2H])c([2H])c2c(sc3c(-c4nc(-c5ccccc5)nc(-c5ccccc5)n4)c([2H])c([2H])c(-c4ccc5c6ccccc6n(-c6ccccc6)c5c4)c32)c1[2H]]

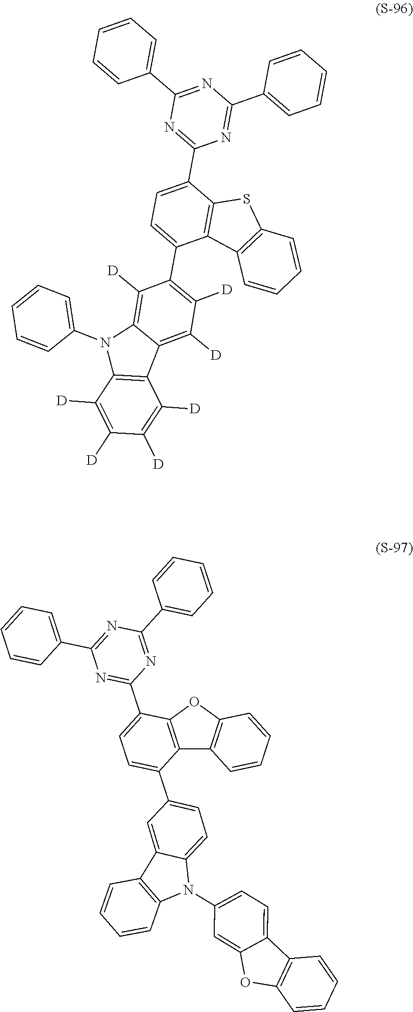 [SMILES: [2H]c1c([2H])c([2H])c2c(c1[2H])c1c([2H])c([2H])c(-c3ccc(-c4nc(-c5ccccc5)nc(-c5ccccc5)n4)c4sc5ccccc5c34)c([2H])c1n2-c1ccccc1 ; c1ccc(-c2nc(-c3ccccc3)nc(-c3ccc(-c4ccc5c(c4)c4ccccc4n5-c4ccc5c(c4)oc4ccccc45)c4c3oc3ccccc34)n2)cc1]

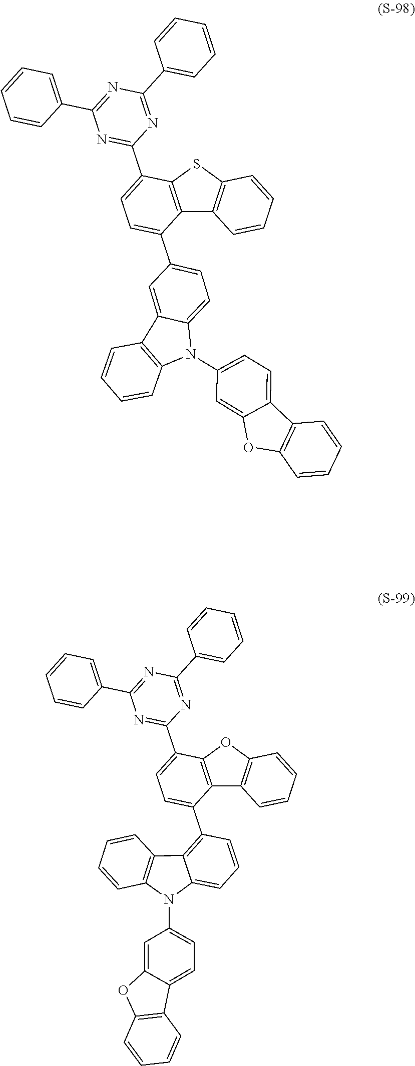 [SMILES: c1ccc(-c2nc(-c3ccccc3)nc(-c3ccc(-c4ccc5c(c4)c4ccccc4n5-c4ccc5c(c4)oc4ccccc45)c4c3sc3ccccc34)n2)cc1 ; c1ccc(-c2nc(-c3ccccc3)nc(-c3ccc(-c4cccc5c4c4ccccc4n5-c4ccc5c(c4)oc4ccccc45)c4c3oc3ccccc34)n2)cc1]

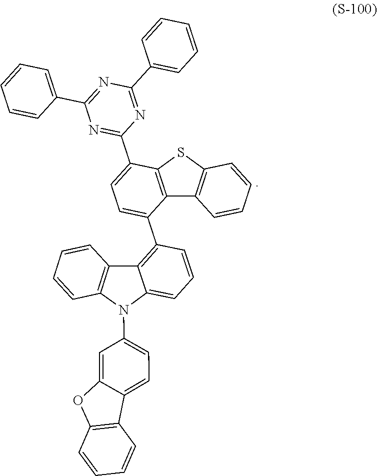 [SMILES: c1ccc(-c2nc(-c3ccccc3)nc(-c3ccc(-c4cccc5c4c4ccccc4n5-c4ccc5c(c4)oc4ccccc45)c4c3sc3ccccc34)n2)cc1]

7. A mixture comprising an organic compound as claimed in claim 1 and at least one organic functional material, wherein the organic functional material is selected from at least one of a hole injection material, a hole transport material, an electron injection material, an electron transport material, a light-emitting auxiliary material, a hole blocking material, a guest material, a host material, and quantum dots.

8. A composition comprising an organic solvent and an organic compound, wherein the organic compound has a chemical structure represented by the following formula (1): 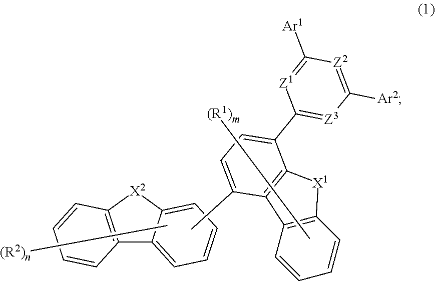
wherein X¹ is selected from O or S;
X² is selected from O, S, or NR³;
Z¹, Z², and Z³ are independently selected from CH or N, and at least one of Z¹, Z², and Z³ is N;
Ar¹ and Ar² are independently selected from a substituted or unsubstituted aromatic group having 6 to 20 carbon atoms, or a substituted or unsubstituted heteroaromatic group having 5 to 20 carbon atoms;
R¹ is selected from a hydrogen atom or a deuterium atom;
R² is selected from a hydrogen atom, a deuterium atom, a substituted or unsubstituted aromatic group having 6 to 20 carbon atoms, or a substituted or unsubstituted heteroaromatic group having 5 to 20 carbon atoms;
R³ is selected from a substituted or unsubstituted aromatic group having 6 to 18 carbon atoms, or a substituted or unsubstituted heteroaromatic group having 5 to 18 carbon atoms;
m is any integer from 1 to 6; and
n is any integer from 1 to 7.

9. The composition of claim 8, wherein Z¹, Z², and Z³ are N.

10. The composition of claim 8, wherein Ar¹ and Ar² are independently selected from a phenyl group, a deuterated phenyl group, a biphenyl group, a triphenyl group, a naphthyl group, a phenanthryl group, a pyridyl group, a pyrimidinyl group, a pyrazinyl group, a quinolinyl group, an isoquinolinyl group, a phenanthrolinyl group, or any combination thereof.

11. The composition of claim 8, wherein R² is selected from a hydrogen atom, a deuterium atom, a phenyl group, a deuterated phenyl group, a biphenyl group, a triphenyl group, a naphthyl group, a phenanthryl group, a pyridyl group, a pyrimidinyl group, a pyrazinyl group, a quinolinyl group, an isoquinolinyl group, a phenanthrolinyl group, or any combination thereof.

12. The composition of claim 8, wherein R³ is selected from a phenyl group, a biphenyl group, a deuterated phenyl group, a pyridyl group, or any combination thereof.

13. An organic electronic device comprising:
a first electrode;
a second electrode disposed opposite to the first electrode; and
an organic functional layer disposed between the first electrode and the second electrode, wherein a material of the organic functional layer comprises an organic compound having a chemical structure represented by the following formula (1): 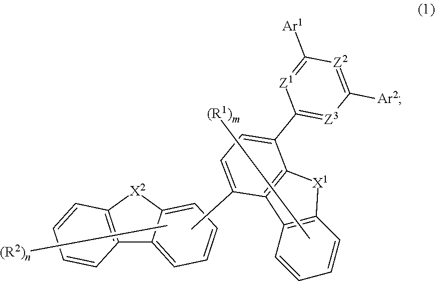
wherein X¹ is selected from O or S;
X² is selected from O, S, or NR³;
Z¹, Z², and Z³ are independently selected from CH or N, and at least one of Z¹, Z², and Z³ is N;
Ar¹ and Ar² are independently selected from a substituted or unsubstituted aromatic group having 6 to 20 carbon atoms, or a substituted or unsubstituted heteroaromatic group having 5 to 20 carbon atoms;
R¹ is selected from a hydrogen atom or a deuterium atom;
R² is selected from a hydrogen atom, a deuterium atom, a substituted or unsubstituted aromatic group having 6 to 20 carbon atoms, or a substituted or unsubstituted heteroaromatic group having 5 to 20 carbon atoms;
R³ is selected from a substituted or unsubstituted aromatic group having 6 to 18 carbon atoms, or a substituted or unsubstituted heteroaromatic group having 5 to 18 carbon atoms;
m is any integer from 1 to 6; and
n is any integer from 1 to 7.

14. The organic electronic device of claim 13, wherein the organic functional layer comprises a light-emitting layer, materials of the light-emitting layer comprise a first host material and a guest material, and the first host material comprises the organic compound.

15. The organic electronic device of claim 14, wherein the materials of the light-emitting layer further comprise a second host material, and the second host material comprises a hole transport type host material; and
wherein the second host material has a highest occupied molecular orbital (HOMO) energy level of −5.5 eV to −5.2 eV.

16. The organic electronic device of claim 13, wherein Z¹, Z², and Z³ are N.

17. The organic electronic device of claim 13, wherein Ar¹ and Ar² are independently selected from a phenyl group, a deuterated phenyl group, a biphenyl group, a triphenyl group, a naphthyl group, a phenanthryl group, a pyridyl group, a pyrimidinyl group, a pyrazinyl group, a quinolinyl group, an isoquinolinyl group, a phenanthrolinyl group, or any combination thereof.

18. The organic electronic device of claim 13, wherein R² is selected from a hydrogen atom, a deuterium atom, a phenyl group, a deuterated phenyl group, a biphenyl group, a triphenyl group, a naphthyl group, a phenanthryl group, a pyridyl group, a pyrimidinyl group, a pyrazinyl group, a quinolinyl group, an isoquinolinyl group, a phenanthrolinyl group, or any combination thereof.

19. The organic electronic device of claim 13, wherein R³ is selected from a phenyl group, a biphenyl group, a deuterated phenyl group, a pyridyl group, or any combination thereof.

20. A display panel comprising an organic electronic device as claimed in claim 13.
